(19) United States
(12) Patent Application Publication
(10) Pub. No.: US 2024/0287062 A1
(43) Pub. Date: Aug. 29, 2024

(54) NAPHTHYRIDONE COMPOUNDS FOR INHIBITION OF RAF KINASES AND/OR BCR-ABL TYROSINE KINASES

(71) Applicant: Enliven Inc., Boulder, CO (US)

(72) Inventors: Joseph P. LYSSIKATOS, Boulder, CO (US); Li REN, Superior, CO (US); Samuel KINTZ, Boulder, CO (US)

(21) Appl. No.: 18/559,317

(22) PCT Filed: May 6, 2022
(86) PCT No.: PCT/US2022/072163

§ 371 Date: Nov. 6, 2023

Related U.S. Application Data
(60) Provisional application No. 63/185,953, filed on May 7, 2021.

(51) Int. Cl. C07D 471/04; A61K 31/4375; A61K 31/444; A61P 35/00

(52) U.S. Cl. CPC C07D 471/04; A61K 31/4375; A61K 31/444; A61P 35/00

(57) ABSTRACT

The present disclosure relates to compounds and compositions for inhibition of RAF serine/threonine protein kinases and inhibition of Bcr-Abl tyrosine kinases, methods of preparing said compounds and compositions, and their use in the treatment of various cancers, such as melanoma, non-small cell lung cancer, and chronic myeloid leukemia (CML).

### CROSS REFERENCE TO RELATED APPLICATION

This applications claims the benefit of priority to U.S. Provisional Patent Application No. 63/185,953, filed May 7, 2021, the disclosure of which is hereby incorporated by reference in its entirety.

### FIELD OF THE INVENTION

Provided herein are compounds and compositions for inhibition of RAF serine/threonine protein kinases and inhibition of Bcr-Abl tyrosine kinases, methods of preparing said compounds and compositions, and their use in the treatment of various cancers, such as melanoma, non-small cell lung cancer, and chronic myeloid leukemia (CML).

### BACKGROUND

The RAF family of serine/threonine protein kinases operate as an essential signaling node within the Ras/Raf/MEK/ERK pathway. Also referred to as the mitogen activated kinase (MAPK) pathway, this signaling cascade is critically involved in the regulation of a diverse array of basic physiological processes. The MAPK pathway is responsive to a variety of stimuli mediated through the input of numerous intracellular second messengers and transmembrane receptors including the receptor tyrosine kinases (RTKs). In the case of the RTKs, upon ligand binding, they act on the MAPK pathway through the recruitment/activation of the RAS GTPases which then bind and activate RAF. RAF then phosphorylates MEK (mitogen activated kinase kinase 1 & 2) at serine residues located within their activation loops that in turn induce certain conformational changes leading to their activation. Activated MEK in turn phosphorylates and activates the ERK kinases (Extracellular Regulated Kinase 1 & 2 also known as MAPK1/2 or mitogen-activated protein kinases 1 & 2) via activation loop phosphorylation. Activated ERK then acts as a broad-based effector of the pathway, modulating the activity of a variety of proteins including other protein kinases, structural proteins, metabolic enzymes and transcription factors that in turn modulate the broad cellular response to these stimuli. Importantly, the primary output of the MAPK pathway is to drive cell growth and proliferation as well as to suppress apoptosis (regulated cell death). Given its central role in the regulation of these processes, it is not surprising that the majority of genetic alterations associated with cellular transformation act entirely or at least in part via the aberrant activation of the MAPK pathway. Therefore, as an essential node in the MAPK pathway, the RAF kinases represent an important therapeutic intervention point for the treatment of a variety of malignancies whose dysregulated growth and survival rely upon this pathway.

Thus, there remains a need for new compounds and compositions for inhibition RAF kinases.

The cytogenetic abnormality known as the Philadelphia chromosome is highly associated with the occurrence of a number of hematological malignancies, including a majority of chronic myeloid leukemias (CML) and a subset of acute lymphoblastic leukemias (Ph+ ALL). The Philadelphia chromosome is a product of a translocation between the breakpoint cluster region (BCR) gene on chromosome 22 and the Abelson (ABL) tyrosine kinase gene on chromosome 9, resulting in the oncogenic fusion gene product Bcr-Abl. The resultant fusion protein both is overexpressed and harbors constitutive kinase activity that then drives the activation of a number of intracellular signaling cascades to induce the uncontrolled cell growth, division and survival associated with oncogenic transformation. Accordingly, therapeutic intervention employing inhibitors of the Bcr-Abl tyrosine kinase represents a cornerstone of the current treatment paradigm for patients with Philadelphia-positive neoplastic disorders.

Imatinib (STI-571), a small molecule Bcr-Abl tyrosine kinase inhibitor (Bcr-Abl TKI), was developed as a highly effective treatment for CML in the early 1990s and is still used today as the first line treatment for CML. However, in more aggressive cases of CML, patients often relapse due to the emergence of resistance. The primary mechanism of this resistance derives from a variety of on-target genetic alterations that drives either aberrant overexpression of the Bcr-Abl fusion or mutations within the Abl kinase domain that reduce imatinib's binding affinity for the active site thereby markedly reducing its inhibitory activity. These alterations can either appear stochastically and represent a sub-population within the initial tumor cell population or arise spontaneously under the selective pressure of inhibitor treatment. Despite efforts to develop additional Bcr-Abl TKIs as second (Nilotinib, Dasatinib, Bosutinib, Radotinib) and third (Ponatinib) lines of therapy to address these resistance mechanisms, all of these Bcr-Abl TKIs suffer from two major drawbacks that limit their clinical utility: (1) limited activity against the panoply of Abl kinase domain resistance mechanisms or (2) poor selectivity for Bcr-Abl versus other protein kinases. Accordingly, these shortcomings often elicit significant dose-limiting toxicities, which then limits these agents' ability to effectively engage the target to achieve clinical efficacy.

Intolerance to Bcr-Abl TKIs is a major clinical challenge. The doses of more than 50% of Ph+ leukemia patients require modification due to adverse events. In fact, approximately 30% of patients are compelled to dose reduce within the first 6 months of treatment. These drug-related side effects appear early in the course of treatment and while manageable in most cases, toxicities persist, significantly impacting the patients' quality of life, resulting in decreased compliance. Accordingly, around 40% of patients discontinue first and second generation Bcr-Abl TKIs within the first 5 years of treatment. All of the approved Bcr-Abl targeted therapies inhibit other tyrosine kinases, which can lead to potentially debilitating side effects. Specifically, potent inhibition of VEGFRs, PDGFRs, c-Kit and/or the c-Src family can lead to dose-limiting side effects in patients. To combat the occurrence of adverse effects, dose reductions, dose interruptions, and even dose discontinuations are common over the course of therapy, but such treatment regimens ultimately result in suboptimal treatment.

Accordingly, there remains a substantial unmet medical need for Bcr-Abl TKIs with improved selectivity to improve tolerability and enhanced potency against the wide array of resistance mechanisms in Philadelphia-positive disorders.

### SUMMARY OF THE INVENTION

Provided herein are compounds and compositions that inhibit RAF kinases and selectively inhibit Bcr-Abl tyrosine kinases and that are useful for treating disorders mediated by RAF kinases and/or Bcr-Abl tyrosine kinases.

In one aspect, provided herein is a compound of formula (I)

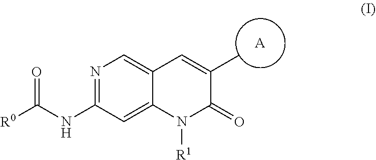

or a pharmaceutically acceptable salt, solvate, hydrate, or co-crystal thereof, or a mixture of any of the foregoing, wherein:
R⁰ is C₁-C₃ alkyl or cyclopropyl, wherein the C₁-C₃ alkyl or cyclopropyl are optionally substituted with 1 to 5 R⁵ groups;
R¹ is H, C₁-C₆ alkyl, C₁-C₆ haloalkyl, C₁-C₆ heteroalkyl, C₃-C₇ cycloalkyl, C₁-C₆ alkyl-CN, 4- to 7-membered heterocycloalkyl, —(X^{a})₀₋₁—(C(R²)R²)₀₋₁—OR²,
   —(X^{a})₀₋₁—(C(R²)R²)₀₋₁—N(R²)R², —(X^{a})₀₋₁—(C(R²)R²)₀₋₁—S(O)₂R²,
   —(X^{a})—(C(R²)R²)₀₋₁—S(O)(═NR²)R², —(X^{a})—C(R²)R²—S(O)₂N(R²)R²,
   —(X^{a})—C(R²)R²—S(O)R², —(X^{a})—C(R²)R²—N(R²)R²S(O)₂R², or
   —(X^{a})—(C(R²)R²)₀₋₁—C(O)N(R²)R²,
   wherein the aliphatic portions of R¹ are optionally substituted with 1 to 5 R³ groups;
X^{a} is C₁-C₆ alkylene, C₁-C₆ heteroalkylene, C₃-C₇ cycloalkylene, or C₄-C₇ heterocycloalkylene, each of which is optionally substituted with 1 to 5 R³ groups;
each R² is independently H, C₁-C₆ alkyl, C₁-C₆ haloalkyl, C₁-C₆ heteroalkyl, C₃-C₇ cycloalkyl, 4- to 7-membered heterocycloalkyl, or 5- to 6-membered heteroaryl,
   wherein the aliphatic and aromatic portions of R² are optionally substituted with 1 to 5 R³ groups, and
   wherein any two R² groups attached to the same nitrogen are optionally taken together to form a 4- to 7-membered heterocyclic ring, or any two R² groups attached to the same carbon atom are optionally taken together to form a 3- to 6-membered carbocyclic ring or a 4- to 6-membered heterocyclic ring optionally containing 1-3 heteroatoms selected from the group consisting of N, O, and S, wherein each carbocyclic or heterocyclic ring is optionally substituted with 1 to 5 R³ groups;
each R³ is independently C₁-C₆ alkyl, C₁-C₆-alkoxy, C₃-C₇ cycloalkyl, C₃-C₇ cycloalkoxy,
   C₁-C₆ haloalkyl, C₁-C₆ alkylamino, C₁-C₆ dialkylamino, 4- to 7-membered heterocycloalkyl, 4- to 7-membered heterocycloalkoxy, 5- to 6-membered heteroaryl, F, Cl, —OH, —NH₂, —NHMe, —NMe₂, —SH, —SMe, —S(O)Me, —S(O)₂Me,
   —S(O)(═NH)Me, —S(O)(═NMe)Me, —CN, —NO₂, —CHF₂, —CF₃, —CF₂Cl, —OCF₃, —OCHF₂, —SCF₃, —SCHF₂, —SF₅, —P(O)(Me)₂, or —N₃;
Ring A is C₆-C₁₀ aryl or 5- to 10-membered heteroaryl,
   wherein the aromatic portions of Ring A are optionally substituted with 1 to 5 R⁴ groups, and
   wherein when Ring A is C₆ aryl or 5- to 6-membered heteroaryl, then any two substituents attached to adjacent atoms of said aryl or heteroaryl are optionally taken together to form a 4- to 6-membered carbocyclic ring or a 4- to 6-membered heterocyclic ring optionally containing 1-3 heteroatoms selected from the group consisting of N, O, and S, wherein each carbocyclic or heterocyclic ring is optionally substituted with 1 to 5 R⁴ groups;
each R⁴ is independently F, Cl, Br, I, —SCF₃, —SCHF₂, —SF₅, —OCF₃, —OR⁶, —N(R⁶)R⁶, —N₃, —OCHF₂, —CF₃, —CF₂Cl, —CHF₂, —CN, —NO₂, —C(═O)C₁-C₆ alkyl, C₁-C₆ alkyl, C₁-C₆ haloalkyl, C₁-C₆ haloalkyl-OH, C₁-C₆ alkyl-OH, C₁-C₆ alkyl-OR⁶, C₁-C₆ alkylene-C₁-C₃ alkoxy, C₁-C₆ alkylene(—OH)—C₁-C₃ alkoxy, C₁-C₆ alkyl-CN, C₁-C₆ heteroalkyl, C₃-C₇ cycloalkyl, C₁-C₆ alkylene-C₃-C₇ cycloalkyl, C₁-C₆ alkylene(—OH)—C₃-C₇ cycloalkyl, or 5- to 6-membered heteroaryl;
each R⁵ is independently F, Cl, —SCF₃, —SCHF₂, —SF₅, —OCF₃, —OR⁶, —N(R⁶)R⁶, —N₃, —OCHF₂, —CF₃, —CF₂Cl, —CHF₂, —CN, —NO₂, C₁-C₆ alkyl, C₁-C₆ haloalkyl, C₁-C₆ heteroalkyl, or C₃-C₇ cycloalkyl,
   wherein the aliphatic portions of R⁵ are optionally substituted with 1 to 3 R³ groups; and
each R⁶ is independently H, C₁-C₆ alkyl, C₁-C₆ haloalkyl, C₁-C₆ heteroalkyl, C₃-C₇ cycloalkyl, C₆ aryl, 5- to 10-membered heteroaryl, or 4- to 7-membered heterocycloalkyl.

In some embodiments, the compound of formula (I) is a compound of formula (I-1),

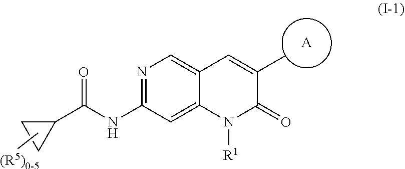

or a pharmaceutically acceptable salt, solvate, hydrate, or co-crystal thereof, or a mixture of any of the foregoing.

In other embodiments, the compound of formula (I) is a compound of formula (I-2):

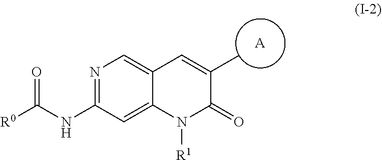

or a pharmaceutically acceptable salt, solvate, hydrate, or co-crystal thereof, or a mixture of any of the foregoing, wherein R¹ is C₁-C₃ alkyl optionally substituted by 1 to 5 R⁵ groups.

In some embodiments, Ring A is selected from the group consisting of:

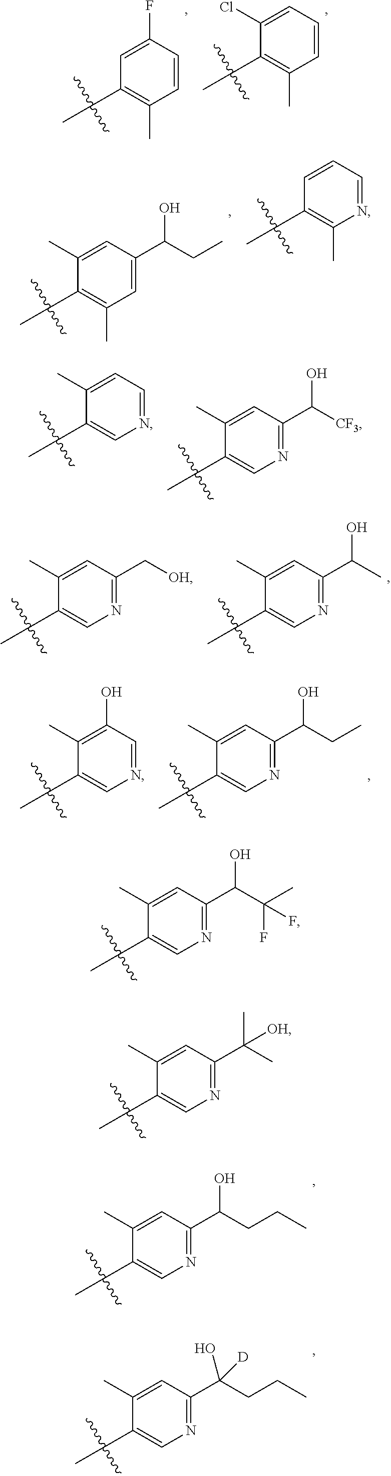

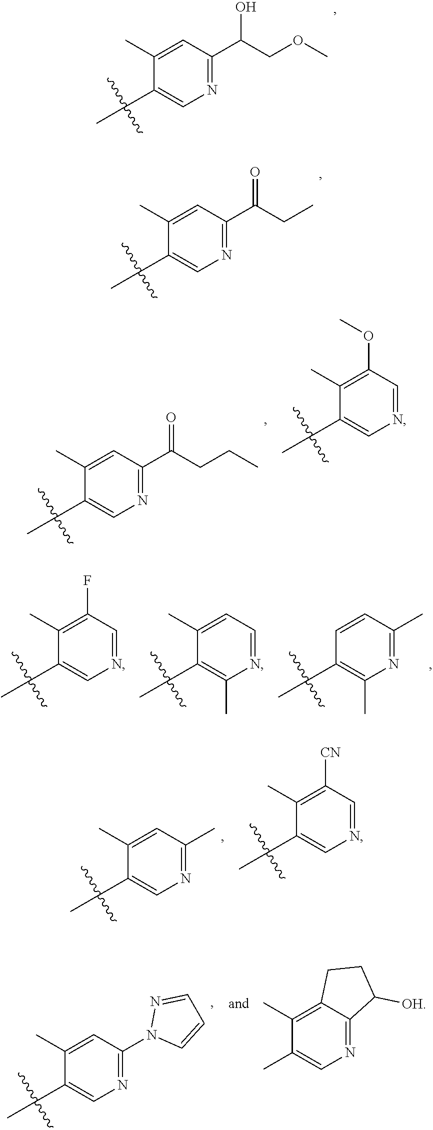

In one aspect, provided is a compound of formula (I-1)

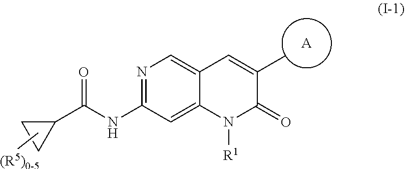

or a pharmaceutically acceptable salt, solvate, hydrate, or co-crystal thereof, or a mixture of any of the foregoing, wherein:
R¹ is H, C₁-C₆ alkyl, C₁-C₆ haloalkyl, C₁-C₆ heteroalkyl, C₃-C₇ cycloalkyl, C₁-C₆ alkyl-CN, 4- to 7-membered heterocycloalkyl, —(X^{a})₀₋₁—(C(R²)R²)₀₋₁—OR²,
   —(X^{a})₀₋₁—(C(R²)R²)₀₋₁—N(R²)R², —(X^{a})₀₋₁—(C(R²)R²)₀₋₁—S(O)₂R²,
   —(X^{a})—(C(R²)R²)₀₋₁—S(O)(═NR²)R², —(X^{a})—C(R²)R²—S(O)₂N(R²)R²,
   —(X^{a})—C(R²)R²—S(O)R², —(X^{a})—C(R²)R²—N(R²)R²S(O)₂R², or
   —(X^{a})—(C(R²)R²)₀₋₁—C(O)N(R²)R²,
   wherein the aliphatic portions of R¹ are optionally substituted with 1 to 5 R³ groups;
X^{a} is C₁-C₆ alkylene, C₁-C₆ heteroalkylene, C₃-C₇ cycloalkylene, or C₄-C₇ heterocycloalkylene, each of which is optionally substituted with 1 to 5 R³ groups;
each R² is independently H, C₁-C₆ alkyl, C₁-C₆ haloalkyl, C₁-C₆ heteroalkyl, C₃-C₇ cycloalkyl, 4- to 7-membered heterocycloalkyl, or 5- to 6-membered heteroaryl,
   wherein the aliphatic and aromatic portions of R² are optionally substituted with 1 to 5 R³ groups, and
   wherein any two R² groups attached to the same nitrogen are optionally taken together to form a 4- to 7-membered heterocyclic ring, or any two R² groups attached to the same carbon atom are optionally taken together to form a 3- to 6-membered carbocyclic ring or a 4- to 6-membered heterocyclic ring optionally containing 1-3 heteroatoms selected from the group consisting of N, O, and S, wherein each carbocyclic or heterocyclic ring is optionally substituted with 1 to 5 R³ groups;
each R³ is independently C₁-C₆ alkyl, C₁-C₆-alkoxy, C₃-C₇ cycloalkyl, C₃-C₇ cycloalkoxy,
   C₁-C₆ haloalkyl, C₁-C₆ alkylamino, C₁-C₆ dialkylamino, 4- to 7-membered heterocycloalkyl, 4- to 7-membered heterocycloalkoxy, 5- to 6-membered heteroaryl, F, Cl, —OH, —NH₂, —NHMe, —NMe₂, —SH, —SMe, —S(O)Me, —S(O)₂Me,
   —S(O)(═NH)Me, —S(O)(═NMe)Me, —CN, —NO₂, —CHF₂, —CF₃, —CF₂Cl, —OCF₃, —OCHF₂, —SCF₃, —SCHF₂, —SF₅, —P(O)(Me)₂, or —N₃;
Ring A is C₆-C₁₀ aryl or 5- to 10-membered heteroaryl,
   wherein the aromatic portions of Ring A are optionally substituted with 1 to 5 R⁴ groups, and
   wherein when Ring A is C₆ aryl or 5- to 6-membered heteroaryl, then any two substituents attached to adjacent atoms of said aryl or heteroaryl are optionally taken together to form a 4- to 6-membered carbocyclic ring or a 4- to 6-membered heterocyclic ring optionally containing 1-3 heteroatoms selected from the group consisting of N, O, and S, wherein each carbocyclic or heterocyclic ring is optionally substituted with 1 to 5 R⁴ groups;
each R⁴ is independently F, Cl, Br, I, —SCF₃, —SCHF₂, —SF₅, —OCF₃, —OR⁶, —N(R⁶)R⁶, —N₃, —OCHF₂, —CF₃, —CF₂Cl, —CHF₂, —CN, —NO₂, C₁-C₆ alkyl, C₁-C₆ haloalkyl, C₁-C₆ haloalkyl-OH, C₁-C₆ alkyl-OH, C₁-C₆ alkyl-CN, C₁-C₆ heteroalkyl, or C₃-C₇ cycloalkyl;
each R⁵ is independently F, Cl, —SCF₃, —SCHF₂, —SF₅, —OCF₃, —OR⁶, —N(R⁶)R⁶, —N₃, —OCHF₂, —CF₃, —CF₂Cl, —CHF₂, —CN, —NO₂, C₁-C₆ alkyl, C₁-C₆ haloalkyl, C₁-C₆ heteroalkyl, or C₃-C₇ cycloalkyl,
   wherein the aliphatic portions of R⁵ are optionally substituted with 1 to 3 R³ groups; and
each R⁶ is independently H, C₁-C₆ alkyl, C₁-C₆ haloalkyl, C₁-C₆ heteroalkyl, C₃-C₇ cycloalkyl, C₆ aryl, 5- to 10-membered heteroaryl, or 4- to 7-membered heterocycloalkyl.

In some embodiments, R¹ is H, C₁-C₆ alkyl, C₁-C₆ haloalkyl, C₁-C₆ heteroalkyl, C₃-C₇ cycloalkyl, C₁-C₆ alkyl-CN, 4- to 7-membered heterocycloalkyl, —(X^{a})₀₋₁—(C(R²)R²)₀₋₁—OR², or
—(X^{a})₀₋₁—(C(R²)R²)₀₋₁—N(R²)R², wherein the aliphatic portions of R¹ are optionally substituted with 1 to 5 R³ groups; X^{a} is C₁-C₆ alkylene, C₁-C₆ heteroalkylene, C₃-C₇ cycloalkylene, or C₄-C₇ heterocycloalkylene, each of which is optionally substituted with 1 to 5 R³ groups;
each R² is independently H, C₁-C₆ alkyl, C₁-C₆ haloalkyl, C₁-C₆ heteroalkyl, C₃-C₇ cycloalkyl, 4- to 7-membered heterocycloalkyl, or 5- to 6-membered heteroaryl, wherein the aliphatic and aromatic portions of R² are optionally substituted with 1 to 5 R³ groups; each R³ is independently C₁-C₆ alkyl, C₁-C₆-alkoxy, C₃-C₇ cycloalkyl, C₃-C₇ cycloalkoxy, C₁-C₆ haloalkyl, C₁-C₆ alkylamino, C₁-C₆ dialkylamino, 4- to 7-membered heterocycloalkyl, 4- to 7-membered heterocycloalkoxy, 5- to 6-membered heteroaryl, F, Cl, —OH, —NH₂, —NHMe, —NMe₂, —SMe, —S(O)Me, —S(O)₂Me, or —CN; Ring A is C₆ aryl or 5- to 6-membered heteroaryl, wherein the aromatic portions of Ring A are optionally substituted with 1 to 5 R⁴ groups; each R⁴ is independently H, F, Cl, Br, I, —SCF₃, —SCHF₂, —SF₅, —OCF₃, —OR⁶, —N(R⁶)R⁶, —N₃, —OCHF₂, —CF₃, —CHF₂, —CN, —NO₂, C₁-C₆ alkyl, C₁-C₆ haloalkyl, C₁-C₆ haloalkyl-OH, C₁-C₆ alkyl-OH, C₁-C₆ alkyl-CN, C₁-C₆ heteroalkyl, or
C₃-C₇ cycloalkyl; each R⁵ is independently F, Cl, —SCF₃, —SCHF₂, —SF₅, —OCF₃, —OR⁶, —N(R⁶)R⁶, —N₃, —OCHF₂, —CF₃, —CHF₂, —CN, —NO₂, C₁-C₆ alkyl, C₁-C₆ haloalkyl, C₁-C₆ heteroalkyl, or C₃-C₇ cycloalkyl, wherein the aliphatic portions of R⁵ are optionally substituted with 1 to 3 R³ groups; and each R⁶ is independently H, C₁-C₆ alkyl, C₁-C₆ haloalkyl, C₁-C₆ heteroalkyl,
C₃-C₇ cycloalkyl, C₆ aryl, 5- to 6-membered heteroaryl, or 4- to 7-membered heterocycloalkyl.

In some embodiments, R¹ is H, C₁-C₆ alkyl, C₁-C₆ haloalkyl, C₁-C₆ alkyl-CN, or C₁-C₆ heteroalkyl, wherein the aliphatic portions of R¹ are optionally substituted with 1 to 5 R³ groups; each R³ is independently C₁-C₆ alkyl, C₁-C₆-alkoxy, C₃-C₇ cycloalkyl, C₃-C₇ cycloalkoxy, C₁-C₆ haloalkyl, C₁-C₆ alkylamino, C₁-C₆ dialkylamino, 4- to 7-membered heterocycloalkyl, 4- to 7-membered heterocycloalkoxy, 5- to 6-membered heteroaryl, F, Cl, —OH, —NH₂, —NHMe, —NMe₂, —SMe, —S(O)Me, —S(O)₂Me, or —CN; Ring A is C₆ aryl or 5- to 6-membered heteroaryl, wherein the aromatic portions of Ring A are optionally substituted with 1 to 5 R⁴ groups; each R⁴ is independently H, F, Cl, —OCF₃, —OR⁶, —N(R⁶)R⁶, —OCHF₂, —CF₃, —CHF₂, —CN, C₁-C₆ alkyl, C₁-C₆ haloalkyl, C₁-C₆ haloalkyl-OH, C₁-C₆ alkyl-CN, C₁-C₆ alkyl-OH, C₁-C₆ heteroalkyl, or C₃-C₇ cycloalkyl; each R⁵ is independently F, —OCF₃, —OR⁶, —N(R⁶)R⁶, —OCHF₂, —CF₃, —CN, C₁-C₆ alkyl, C₁-C₆ haloalkyl, or C₁-C₆ heteroalkyl, wherein the aliphatic portions of R⁵ are optionally substituted with 1 to 3 R³ groups; and each R⁶ is independently H, C₁-C₆ alkyl, C₁-C₆ haloalkyl, C₁-C₆ heteroalkyl, or C₃-C₇ cycloalkyl.

In some embodiments, R¹ is H or C₁-C₆ alkyl optionally substituted with 1 to 5 R³ groups. In some embodiments, R¹ is —CH₃ or —CH₂CH₃.

In some embodiments, Ring A is 6-membered heteroaryl optionally substituted with 1 to 4 R⁴ groups. In some embodiments, Ring A is pyridyl optionally substituted with 1 to 4 R⁴ groups. In some embodiments, Ring A is

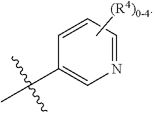

In some embodiments, each R⁴ is independently F, C₁-C₆ alkyl, —OR⁶, —CN, —CH₂CN, —CH₂CH₂CN, C₁-C₆ alkyl-OH, or C₁-C₆ haloalkyl-OH; and each R⁶ is independently H or C₁-C₆ alkyl. In some embodiments, each R⁴ is independently F, —CH₃, —OCH₃, —OH, —CN, —CH₂OH,
—CH(OH)CH₃, —CH(OH)CH₂CH₃, —CH(OH)CF₂CH₃, —CH(OH)CH₂CH₂CH₃, —CD(OH)CH₂CH₂CH₃, —C(═O)CH₂CH₃, —C(═O)CH₂CH₂CH₃, —CH(OH)CH₂OCH₃, —C(CH₃)₂OH, or —CH(OH)CF₃. In some embodiments, each R⁴ is independently F, —CH₃, —OCH₃, —OH, —CN, —CH₂OH, —CH(OH)CH₃, —CH(OH)CH₂CH₃, —C(CH₃)₂OH, or —CH(OH)CF₃.

In some embodiments, Ring A is selected from the group consisting of:

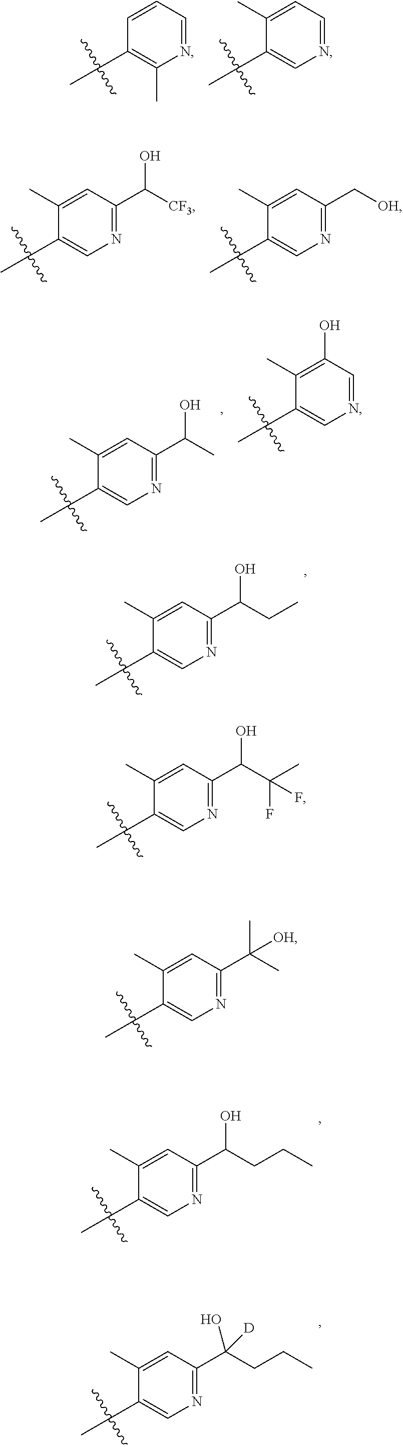

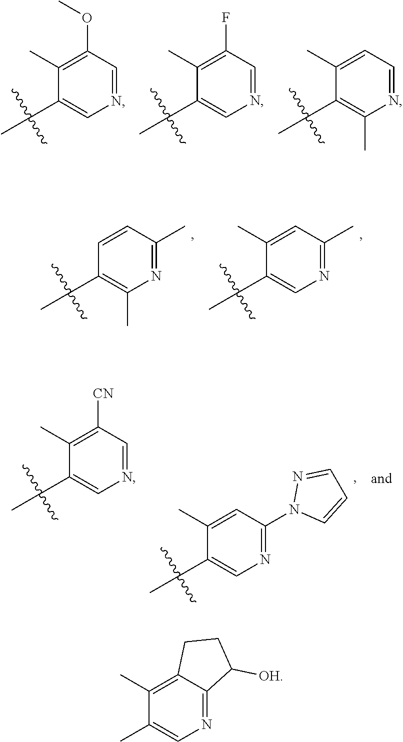

In some embodiments, Ring A is selected from the group consisting of:

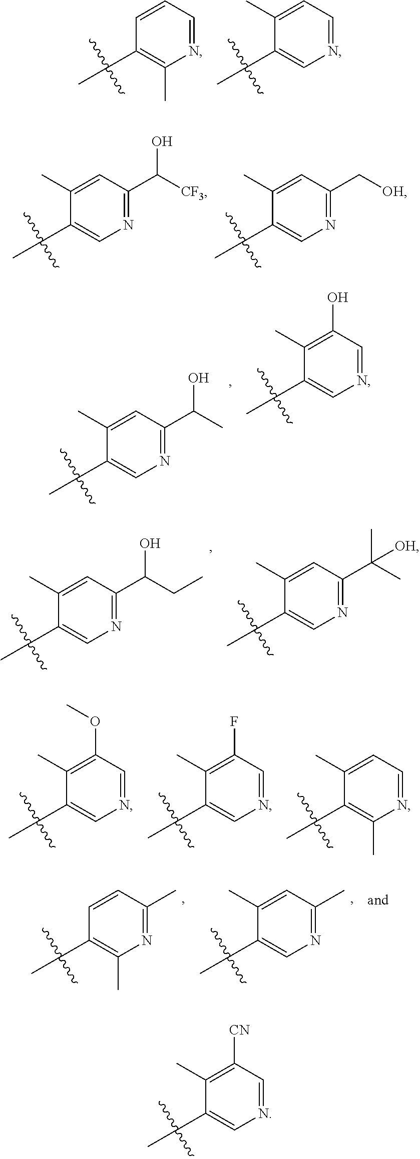

In some embodiments, R⁵, if present, is F.

In some embodiments,

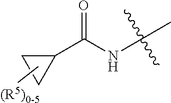

of formula (I) is

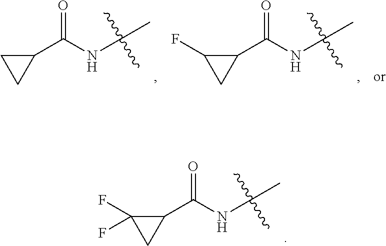 [SMILES: *NC(=O)C1CC1 ; *NC(=O)C1CC1F ; *NC(=O)C1CC1(F)F]

Also provided herein is a compound which is selected from the group consisting of:

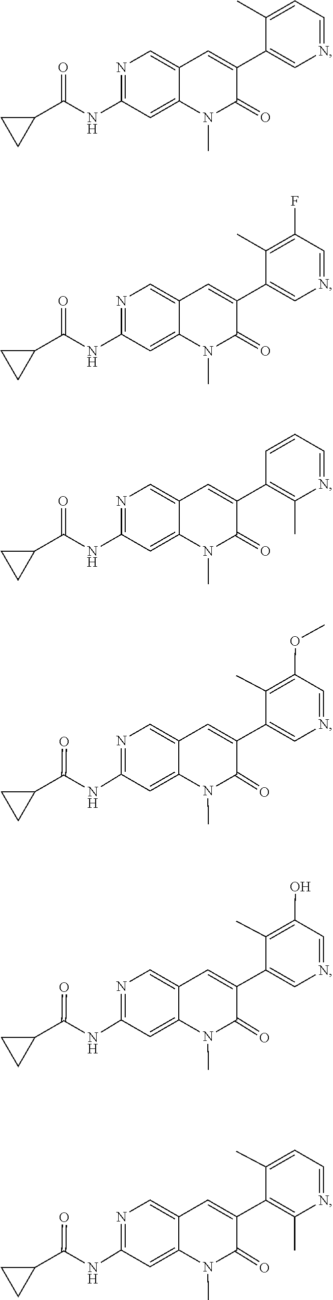 [SMILES: Cc1ccncc1-c1cc2cnc(NC(=O)C3CC3)cc2n(C)c1=O ; Cc1c(F)cncc1-c1cc2cnc(NC(=O)C3CC3)cc2n(C)c1=O ; Cc1ncccc1-c1cc2cnc(NC(=O)C3CC3)cc2n(C)c1=O ; COc1cncc(-c2cc3cnc(NC(=O)C4CC4)cc3n(C)c2=O)c1C ; Cc1c(O)cncc1-c1cc2cnc(NC(=O)C3CC3)cc2n(C)c1=O ; Cc1ccnc(C)c1-c1cc2cnc(NC(=O)C3CC3)cc2n(C)c1=O]

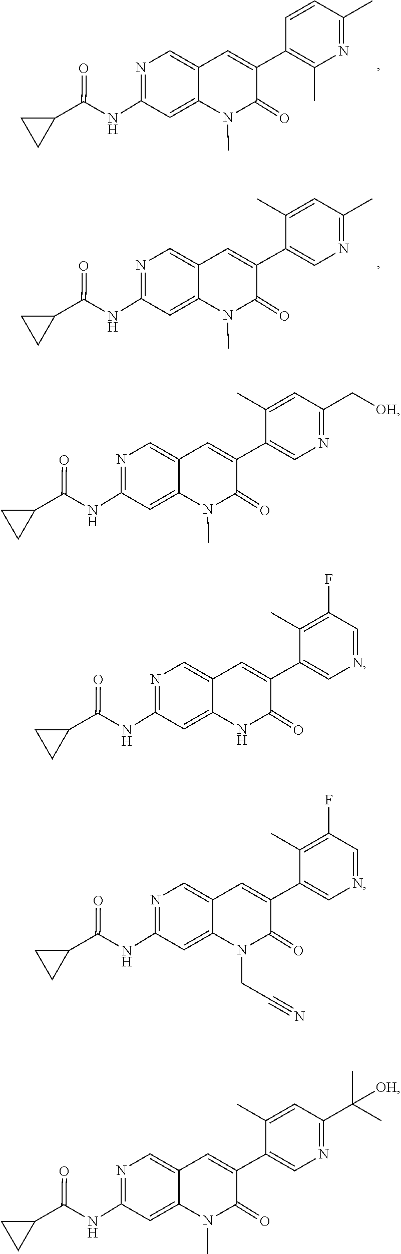

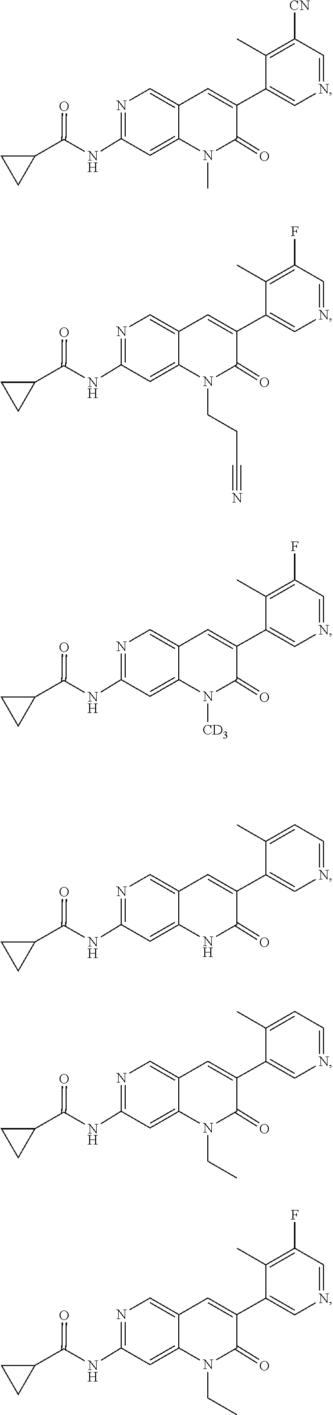 [SMILES: Cc1c(C#N)cncc1-c1cc2cnc(NC(=O)C3CC3)cc2n(C)c1=O ; Cc1c(F)cncc1-c1cc2cnc(NC(=O)C3CC3)cc2n(CCC#N)c1=O ; [2H]C([2H])([2H])n1c(=O)c(-c2cncc(F)c2C)cc2cnc(NC(=O)C3CC3)cc21 ; Cc1ccncc1-c1cc2cnc(NC(=O)C3CC3)cc2[nH]c1=O ; CCn1c(=O)c(-c2cnccc2C)cc2cnc(NC(=O)C3CC3)cc21 ; CCn1c(=O)c(-c2cncc(F)c2C)cc2cnc(NC(=O)C3CC3)cc21]

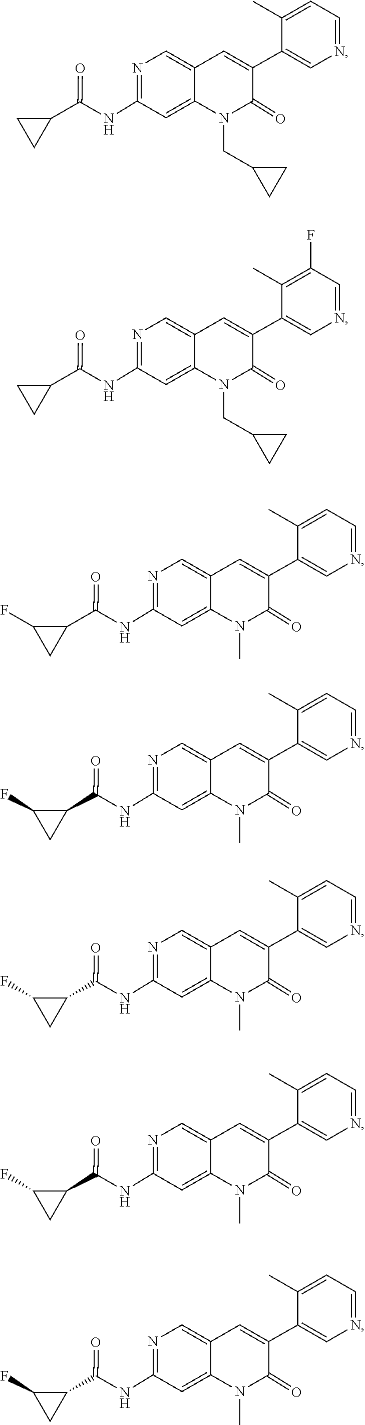

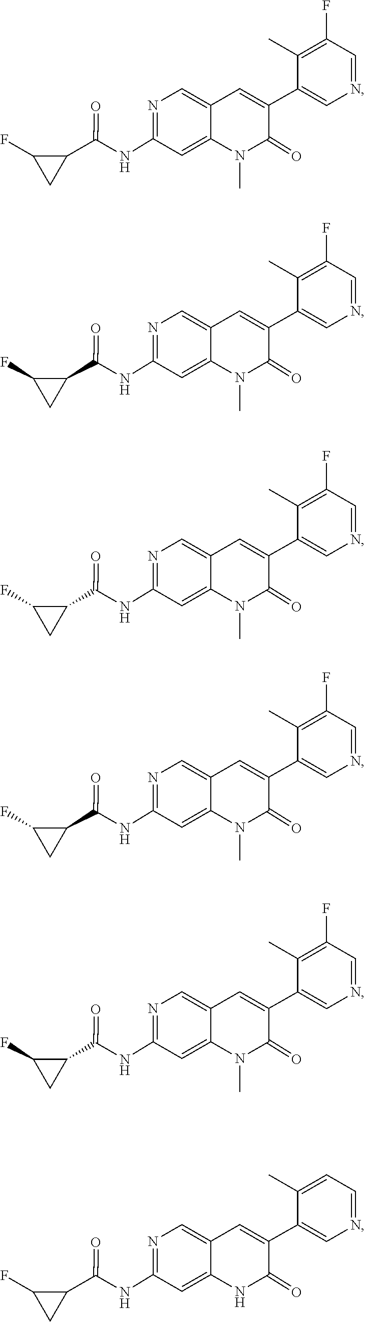 [SMILES: Cc1c(F)cncc1-c1cc2cnc(NC(=O)C3CC3F)cc2n(C)c1=O ; Cc1c(F)cncc1-c1cc2cnc(NC(=O)[C@H]3C[C@H]3F)cc2n(C)c1=O ; Cc1c(F)cncc1-c1cc2cnc(NC(=O)[C@@H]3C[C@@H]3F)cc2n(C)c1=O ; Cc1c(F)cncc1-c1cc2cnc(NC(=O)[C@H]3C[C@@H]3F)cc2n(C)c1=O ; Cc1c(F)cncc1-c1cc2cnc(NC(=O)[C@@H]3C[C@H]3F)cc2n(C)c1=O ; Cc1ccncc1-c1cc2cnc(NC(=O)C3CC3F)cc2[nH]c1=O]

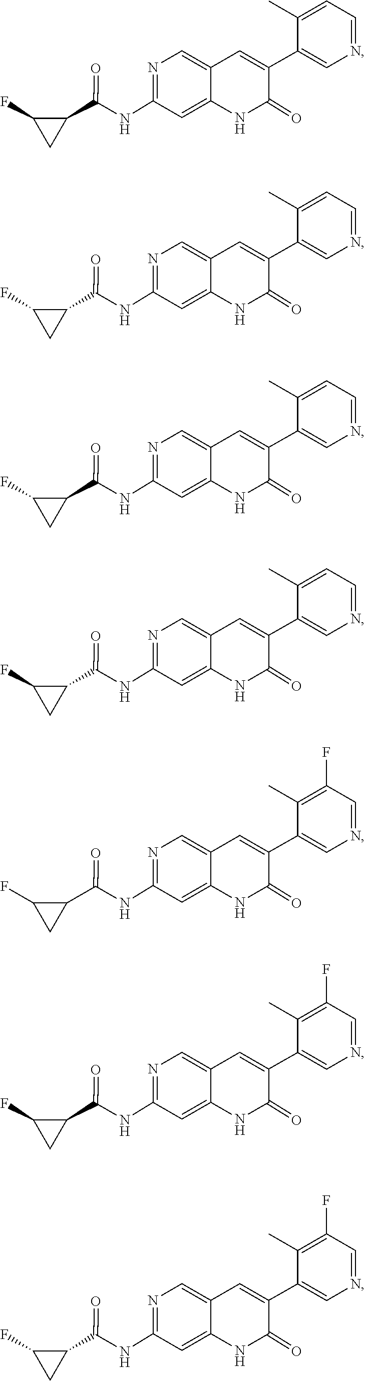

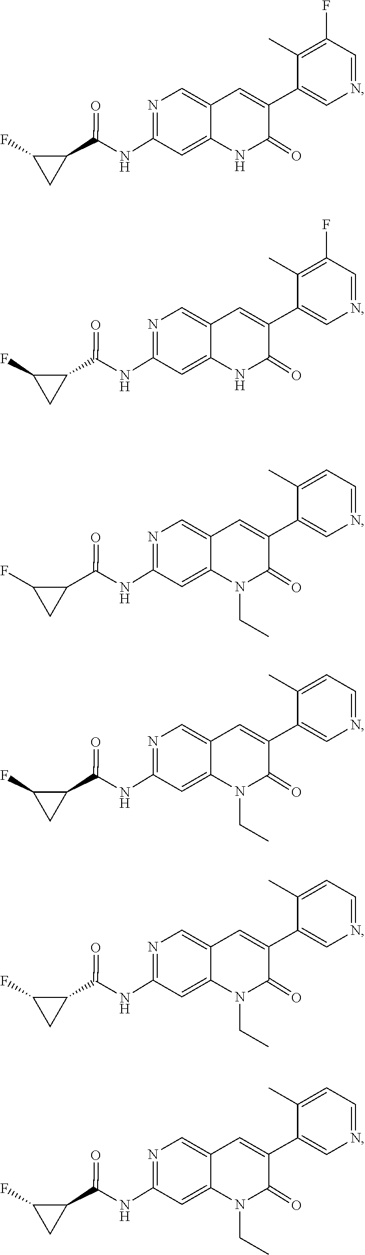

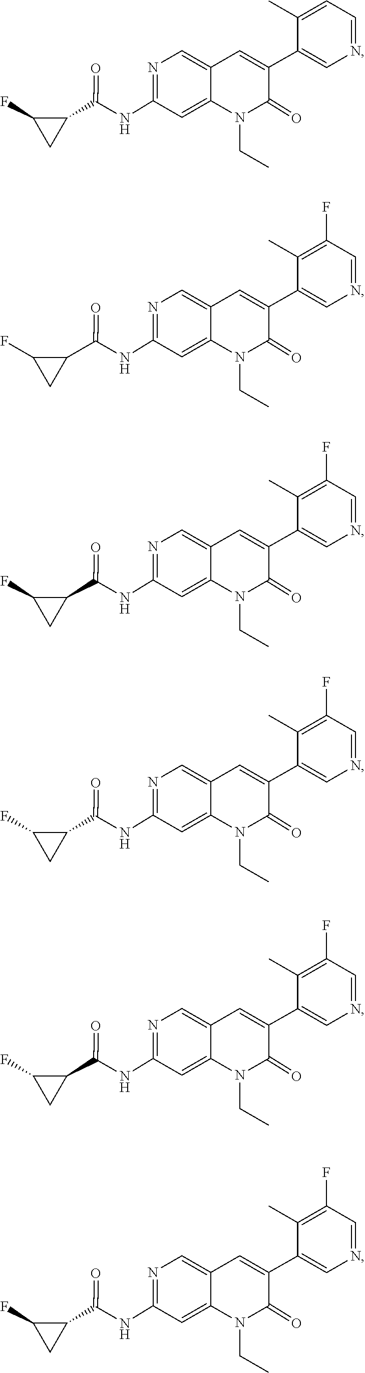 [SMILES: CCn1c(=O)c(-c2cnccc2C)cc2cnc(NC(=O)[C@@H]3C[C@H]3F)cc21 ; CCn1c(=O)c(-c2cncc(F)c2C)cc2cnc(NC(=O)C3CC3F)cc21 ; CCn1c(=O)c(-c2cncc(F)c2C)cc2cnc(NC(=O)[C@H]3C[C@H]3F)cc21 ; CCn1c(=O)c(-c2cncc(F)c2C)cc2cnc(NC(=O)[C@@H]3C[C@@H]3F)cc21 ; CCn1c(=O)c(-c2cncc(F)c2C)cc2cnc(NC(=O)[C@H]3C[C@@H]3F)cc21 ; CCn1c(=O)c(-c2cncc(F)c2C)cc2cnc(NC(=O)[C@@H]3C[C@H]3F)cc21]

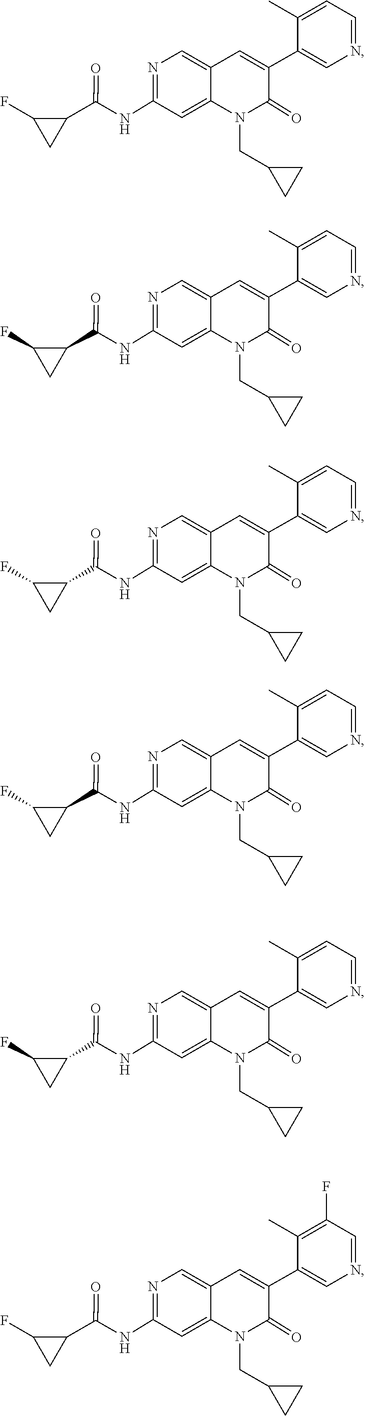

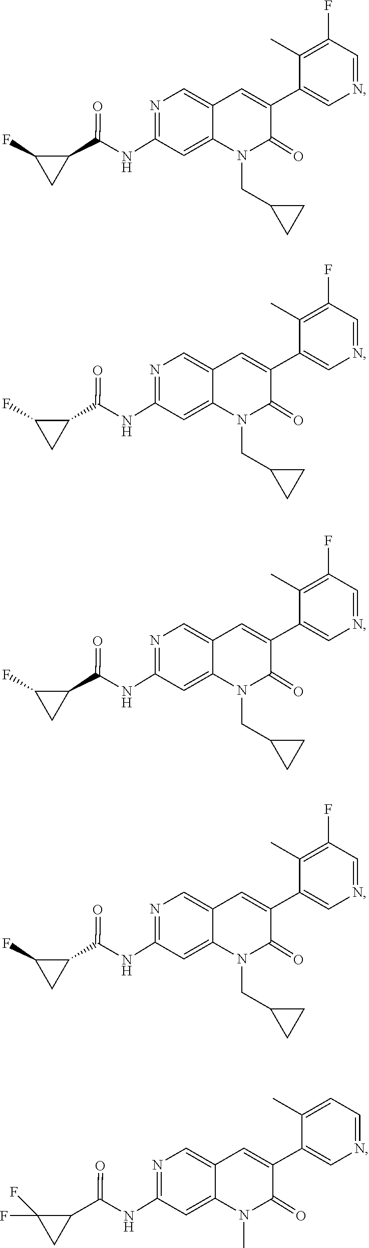 [SMILES: Cc1c(F)cncc1-c1cc2cnc(NC(=O)[C@H]3C[C@H]3F)cc2n(CC2CC2)c1=O ; Cc1c(F)cncc1-c1cc2cnc(NC(=O)[C@@H]3C[C@@H]3F)cc2n(CC2CC2)c1=O ; Cc1c(F)cncc1-c1cc2cnc(NC(=O)[C@H]3C[C@@H]3F)cc2n(CC2CC2)c1=O ; Cc1c(F)cncc1-c1cc2cnc(NC(=O)[C@@H]3C[C@H]3F)cc2n(CC2CC2)c1=O ; Cc1ccncc1-c1cc2cnc(NC(=O)C3CC3(F)F)cc2n(C)c1=O]

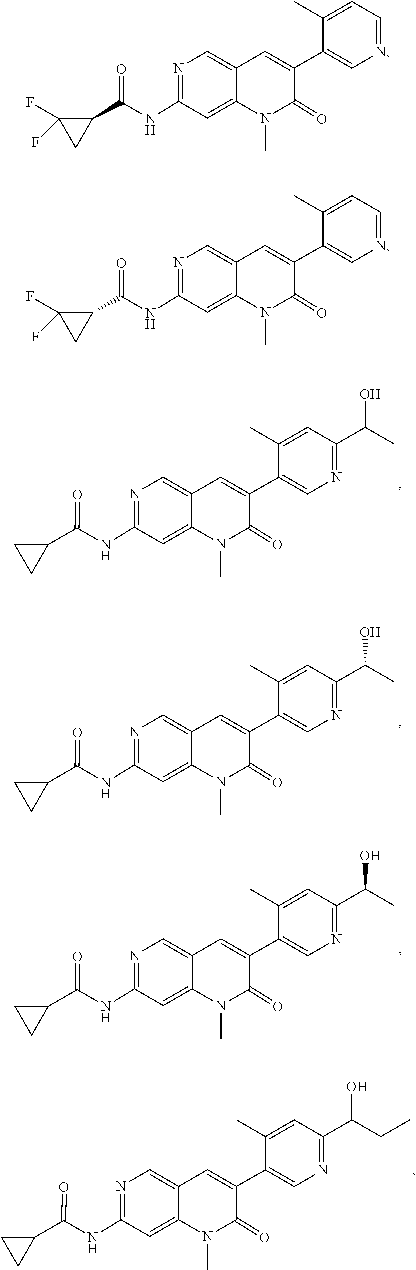 [SMILES: Cc1ccncc1-c1cc2cnc(NC(=O)[C@H]3CC3(F)F)cc2n(C)c1=O ; Cc1ccncc1-c1cc2cnc(NC(=O)[C@@H]3CC3(F)F)cc2n(C)c1=O ; Cc1cc(C(C)O)ncc1-c1cc2cnc(NC(=O)C3CC3)cc2n(C)c1=O ; Cc1cc([C@@H](C)O)ncc1-c1cc2cnc(NC(=O)C3CC3)cc2n(C)c1=O ; Cc1cc([C@H](C)O)ncc1-c1cc2cnc(NC(=O)C3CC3)cc2n(C)c1=O ; CCC(O)c1cc(C)c(-c2cc3cnc(NC(=O)C4CC4)cc3n(C)c2=O)cn1]

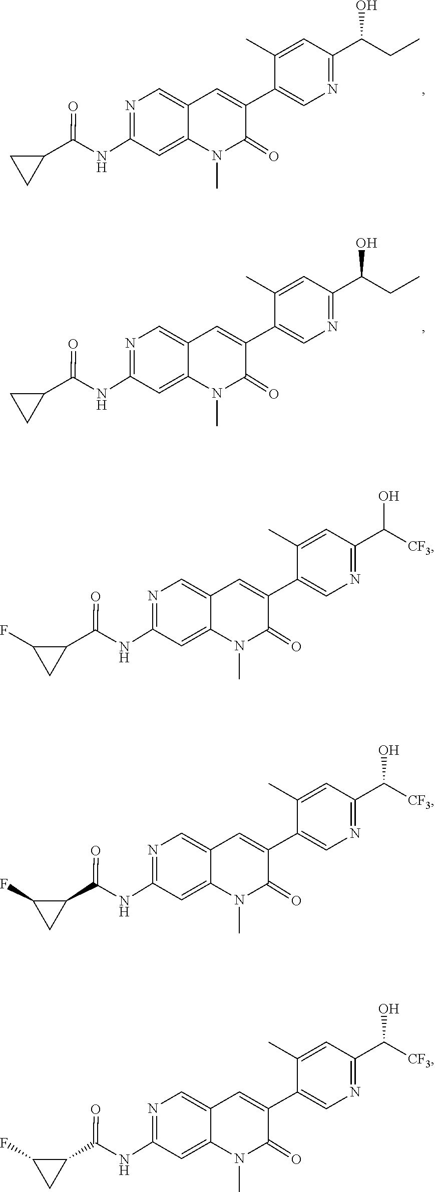 [SMILES: CC[C@@H](O)c1cc(C)c(-c2cc3cnc(NC(=O)C4CC4)cc3n(C)c2=O)cn1 ; CC[C@H](O)c1cc(C)c(-c2cc3cnc(NC(=O)C4CC4)cc3n(C)c2=O)cn1 ; Cc1cc(C(O)C(F)(F)F)ncc1-c1cc2cnc(NC(=O)C3CC3F)cc2n(C)c1=O ; Cc1cc([C@H](O)C(F)(F)F)ncc1-c1cc2cnc(NC(=O)[C@H]3C[C@H]3F)cc2n(C)c1=O ; Cc1cc([C@H](O)C(F)(F)F)ncc1-c1cc2cnc(NC(=O)[C@@H]3C[C@@H]3F)cc2n(C)c1=O]

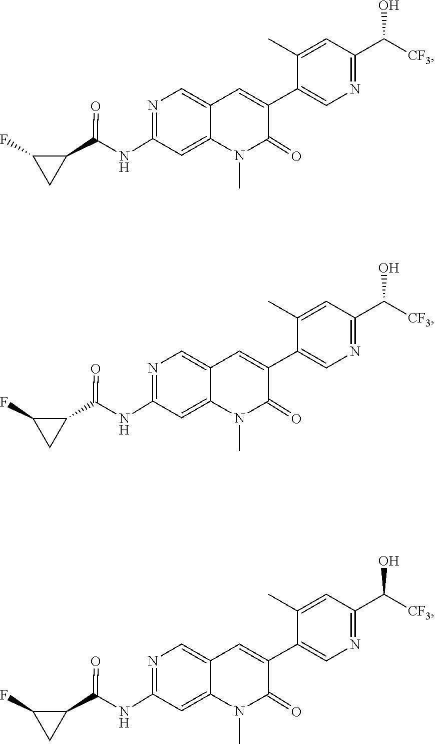 [SMILES: Cc1cc([C@H](O)C(F)(F)F)ncc1-c1cc2cnc(NC(=O)[C@H]3C[C@@H]3F)cc2n(C)c1=O ; Cc1cc([C@H](O)C(F)(F)F)ncc1-c1cc2cnc(NC(=O)[C@@H]3C[C@H]3F)cc2n(C)c1=O ; Cc1cc([C@@H](O)C(F)(F)F)ncc1-c1cc2cnc(NC(=O)[C@H]3C[C@H]3F)cc2n(C)c1=O]

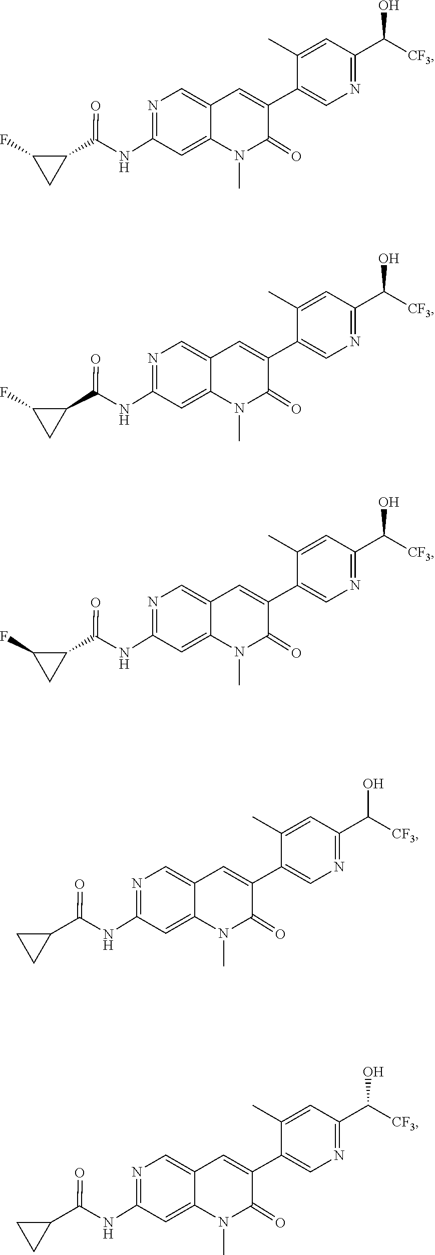 [SMILES: Cc1cc([C@@H](O)C(F)(F)F)ncc1-c1cc2cnc(NC(=O)[C@@H]3C[C@@H]3F)cc2n(C)c1=O ; Cc1cc([C@@H](O)C(F)(F)F)ncc1-c1cc2cnc(NC(=O)[C@H]3C[C@@H]3F)cc2n(C)c1=O ; Cc1cc([C@@H](O)C(F)(F)F)ncc1-c1cc2cnc(NC(=O)[C@@H]3C[C@H]3F)cc2n(C)c1=O ; Cc1cc(C(O)C(F)(F)F)ncc1-c1cc2cnc(NC(=O)C3CC3)cc2n(C)c1=O ; Cc1cc([C@H](O)C(F)(F)F)ncc1-c1cc2cnc(NC(=O)C3CC3)cc2n(C)c1=O]

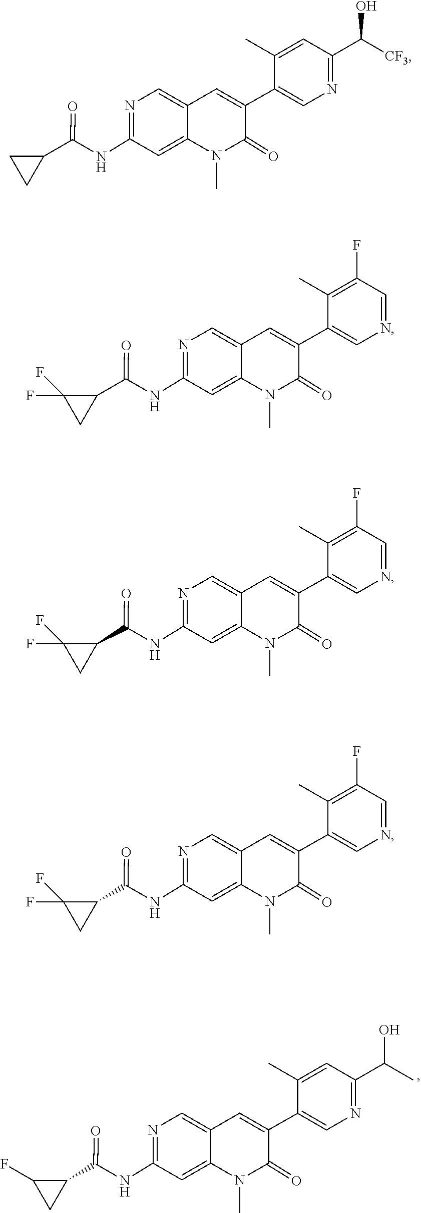 [SMILES: Cc1cc([C@@H](O)C(F)(F)F)ncc1-c1cc2cnc(NC(=O)C3CC3)cc2n(C)c1=O ; Cc1c(F)cncc1-c1cc2cnc(NC(=O)C3CC3(F)F)cc2n(C)c1=O ; Cc1c(F)cncc1-c1cc2cnc(NC(=O)[C@H]3CC3(F)F)cc2n(C)c1=O ; Cc1c(F)cncc1-c1cc2cnc(NC(=O)[C@@H]3CC3(F)F)cc2n(C)c1=O ; Cc1cc(C(C)O)ncc1-c1cc2cnc(NC(=O)[C@@H]3CC3F)cc2n(C)c1=O]

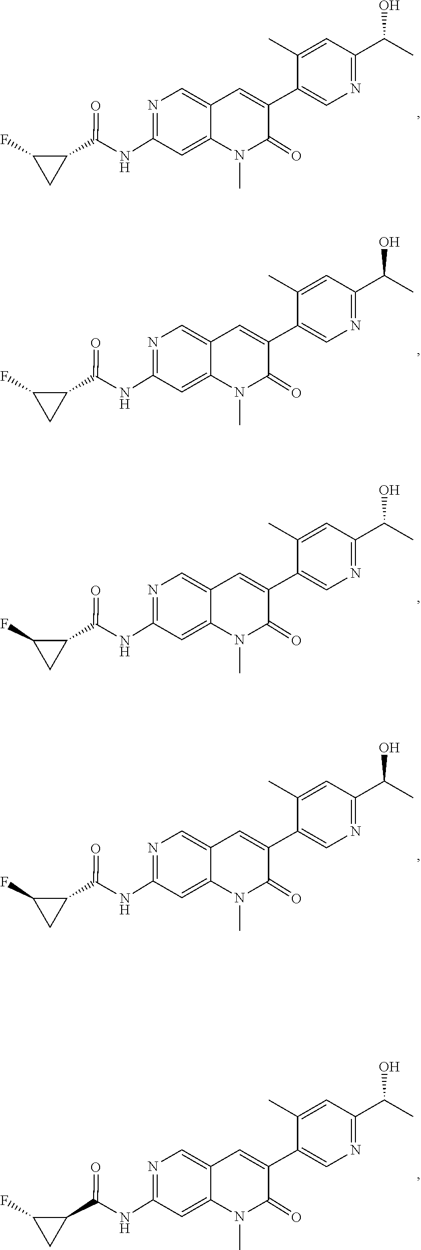 [SMILES: Cc1cc([C@@H](C)O)ncc1-c1cc2cnc(NC(=O)[C@@H]3C[C@@H]3F)cc2n(C)c1=O ; Cc1cc([C@H](C)O)ncc1-c1cc2cnc(NC(=O)[C@@H]3C[C@@H]3F)cc2n(C)c1=O ; Cc1cc([C@@H](C)O)ncc1-c1cc2cnc(NC(=O)[C@@H]3C[C@H]3F)cc2n(C)c1=O ; Cc1cc([C@H](C)O)ncc1-c1cc2cnc(NC(=O)[C@@H]3C[C@H]3F)cc2n(C)c1=O ; Cc1cc([C@@H](C)O)ncc1-c1cc2cnc(NC(=O)[C@H]3C[C@@H]3F)cc2n(C)c1=O]

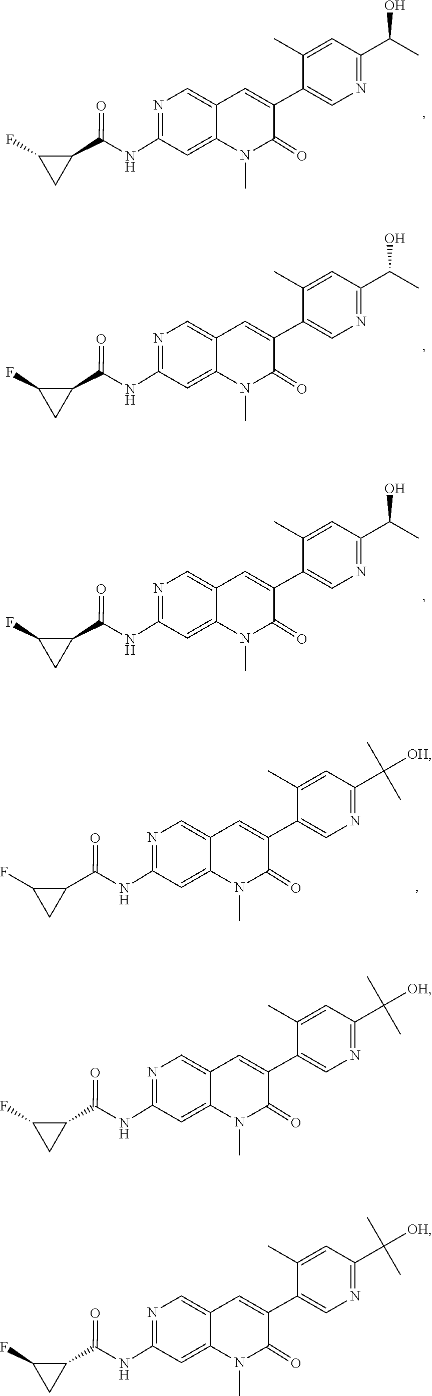 [SMILES: Cc1cc([C@H](C)O)ncc1-c1cc2cnc(NC(=O)[C@H]3C[C@@H]3F)cc2n(C)c1=O ; Cc1cc([C@@H](C)O)ncc1-c1cc2cnc(NC(=O)[C@H]3C[C@H]3F)cc2n(C)c1=O ; Cc1cc([C@H](C)O)ncc1-c1cc2cnc(NC(=O)[C@H]3C[C@H]3F)cc2n(C)c1=O ; Cc1cc(C(C)(C)O)ncc1-c1cc2cnc(NC(=O)C3CC3F)cc2n(C)c1=O ; Cc1cc(C(C)(C)O)ncc1-c1cc2cnc(NC(=O)[C@@H]3C[C@@H]3F)cc2n(C)c1=O ; Cc1cc(C(C)(C)O)ncc1-c1cc2cnc(NC(=O)[C@@H]3C[C@H]3F)cc2n(C)c1=O]

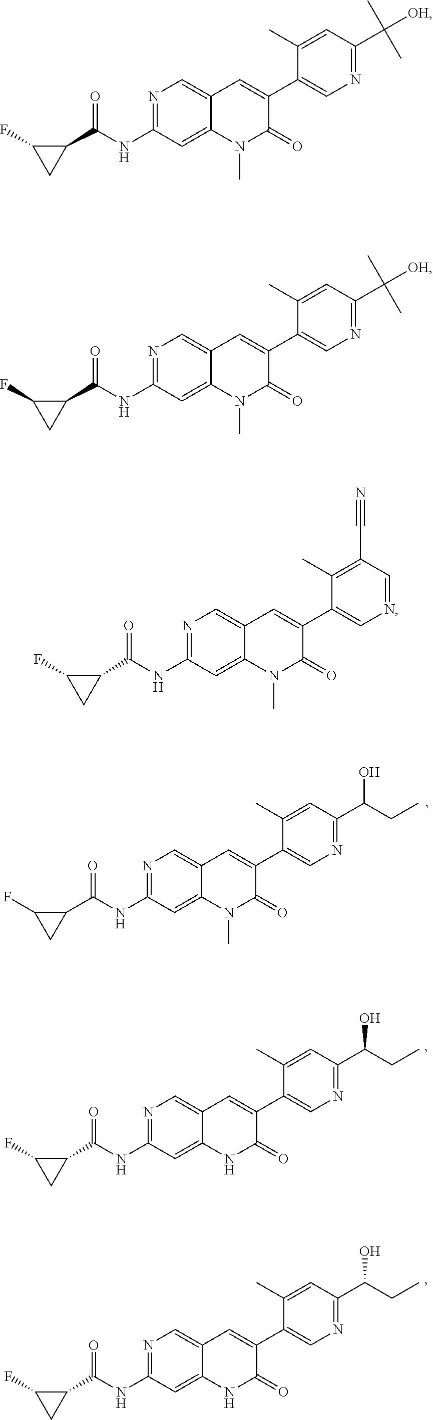 [SMILES: Cc1cc(C(C)(C)O)ncc1-c1cc2cnc(NC(=O)[C@H]3C[C@@H]3F)cc2n(C)c1=O ; Cc1cc(C(C)(C)O)ncc1-c1cc2cnc(NC(=O)[C@H]3C[C@H]3F)cc2n(C)c1=O ; Cc1c(C#N)cncc1-c1cc2cnc(NC(=O)[C@@H]3C[C@@H]3F)cc2n(C)c1=O ; CCC(O)c1cc(C)c(-c2cc3cnc(NC(=O)C4CC4F)cc3n(C)c2=O)cn1 ; CC[C@H](O)c1cc(C)c(-c2cc3cnc(NC(=O)[C@@H]4C[C@@H]4F)cc3[nH]c2=O)cn1 ; CC[C@@H](O)c1cc(C)c(-c2cc3cnc(NC(=O)[C@@H]4C[C@@H]4F)cc3[nH]c2=O)cn1]

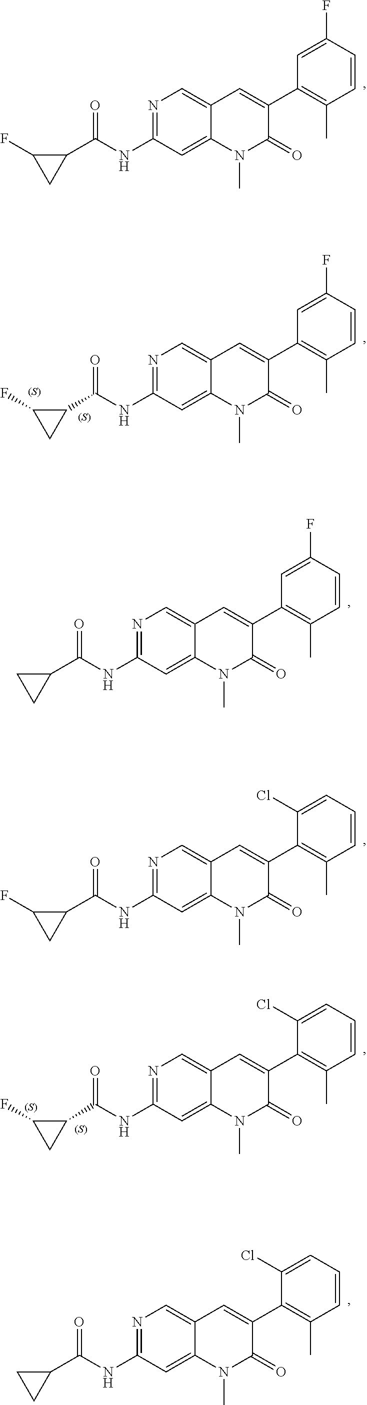

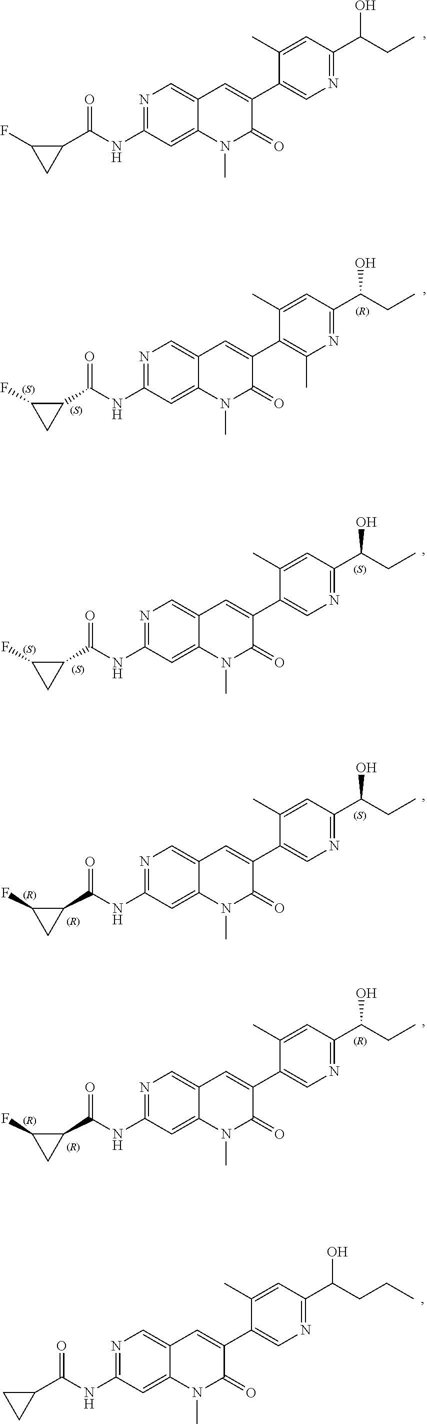

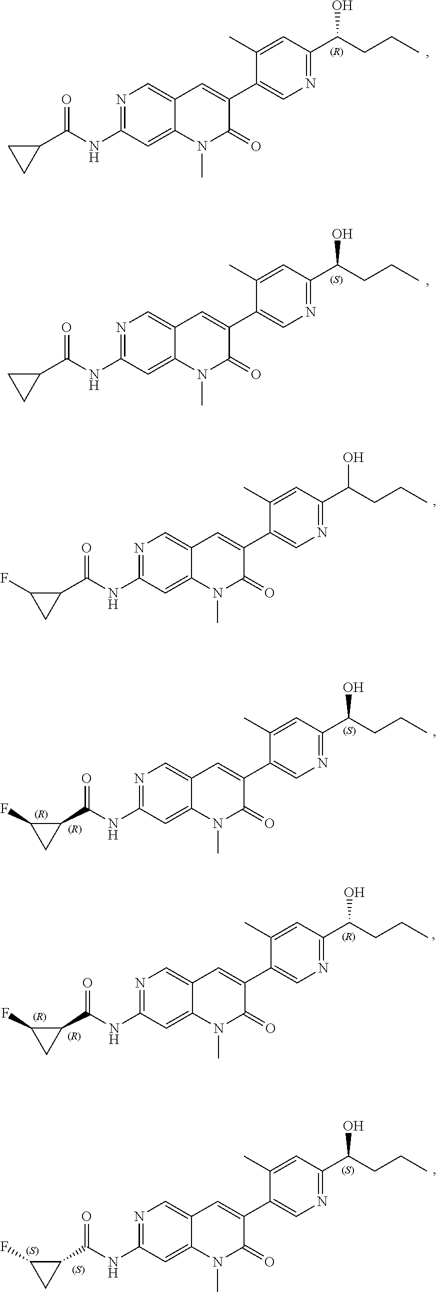

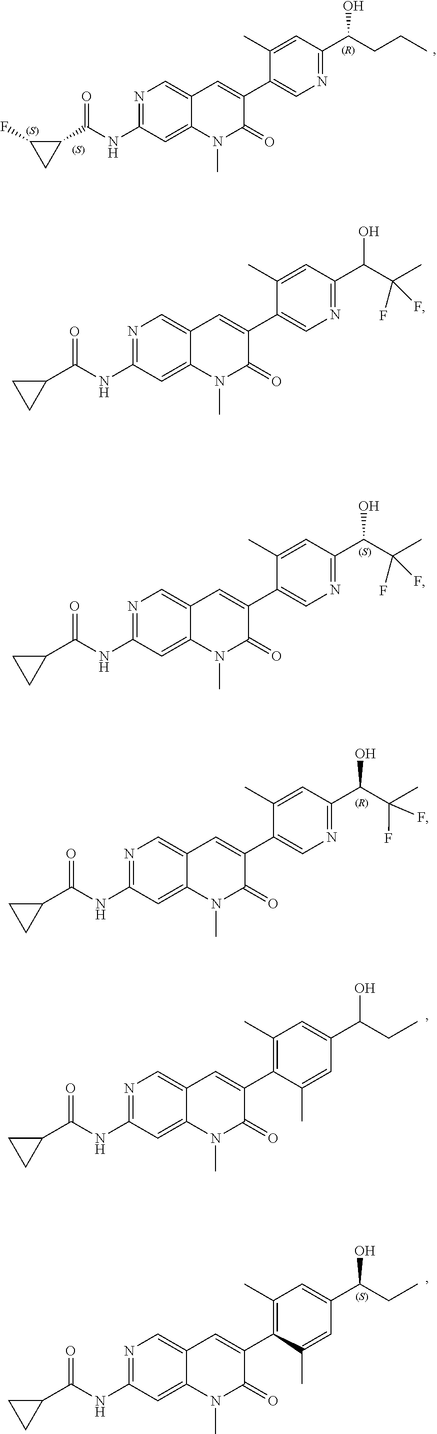

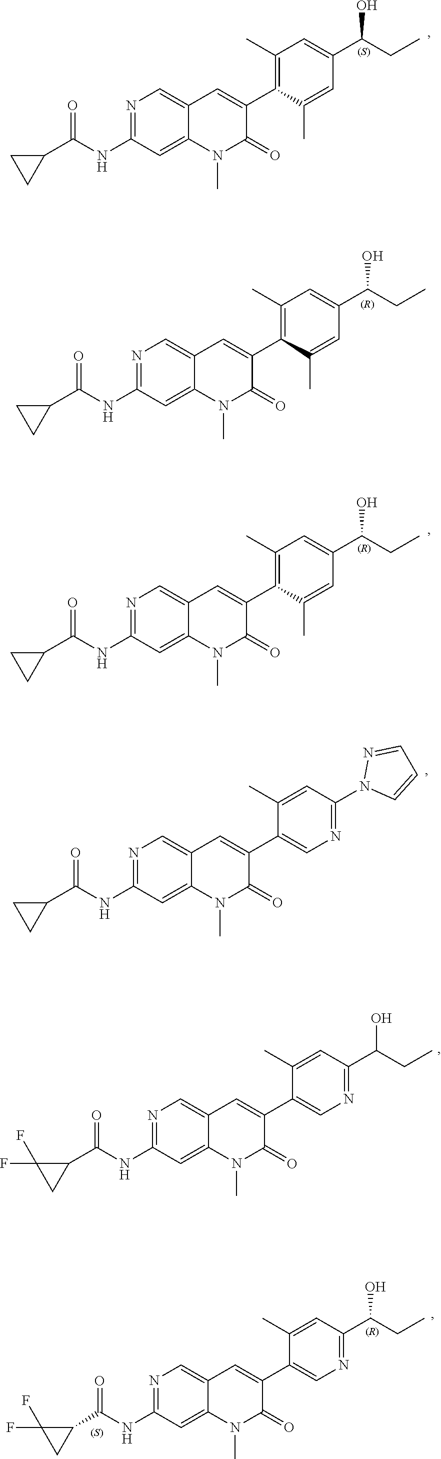

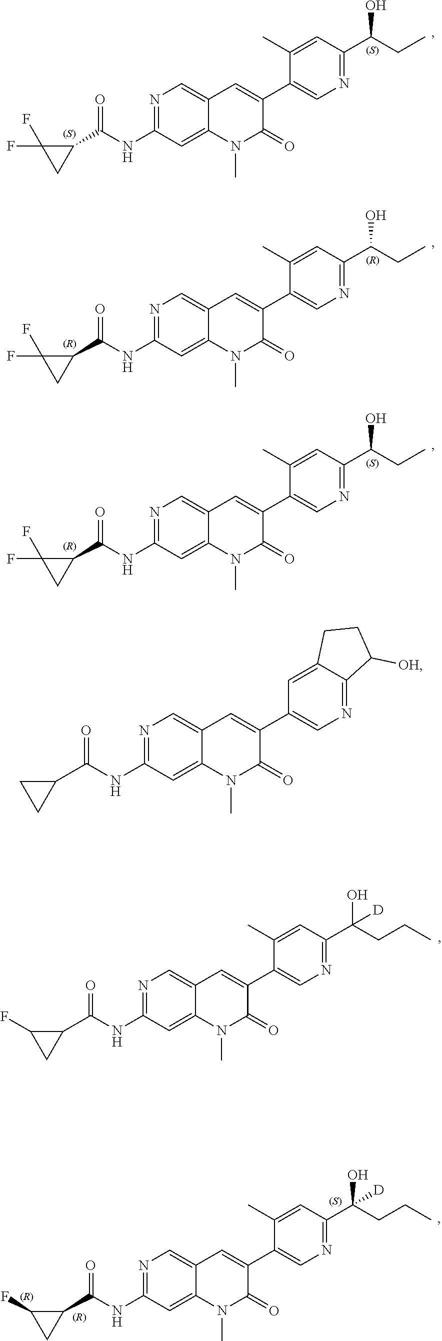

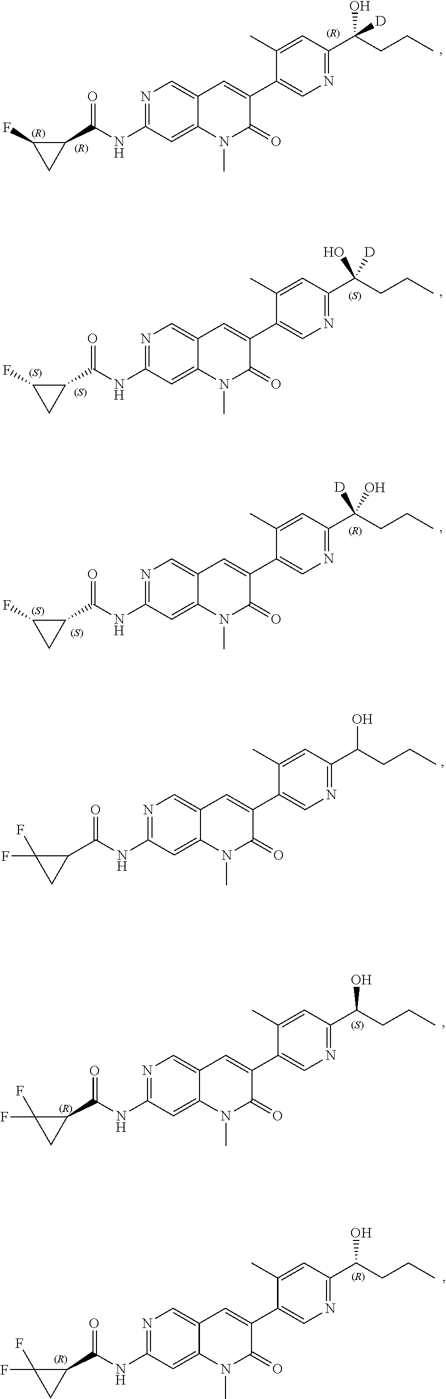

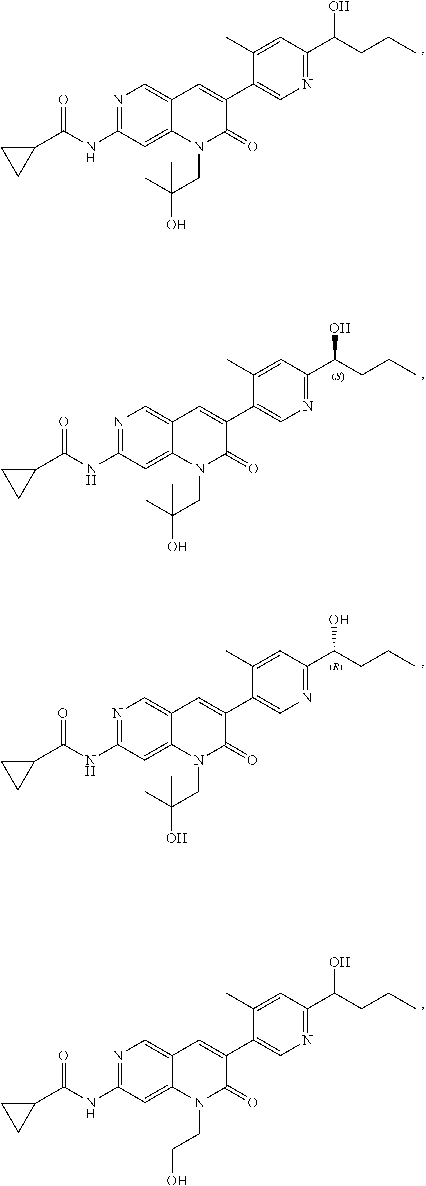

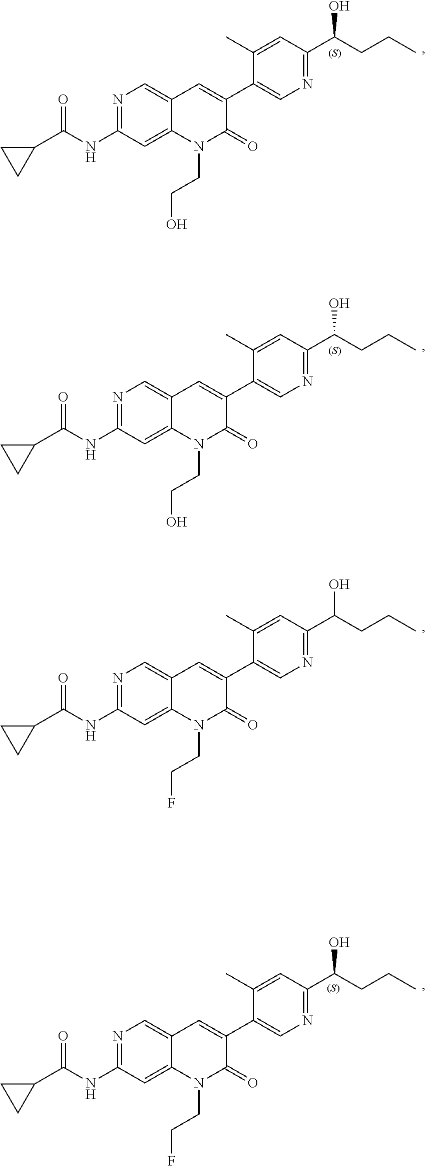

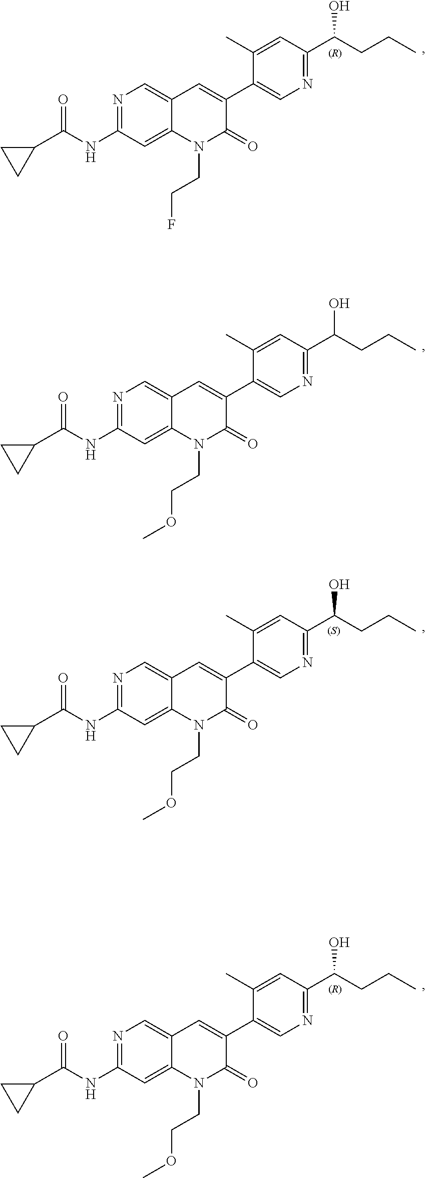

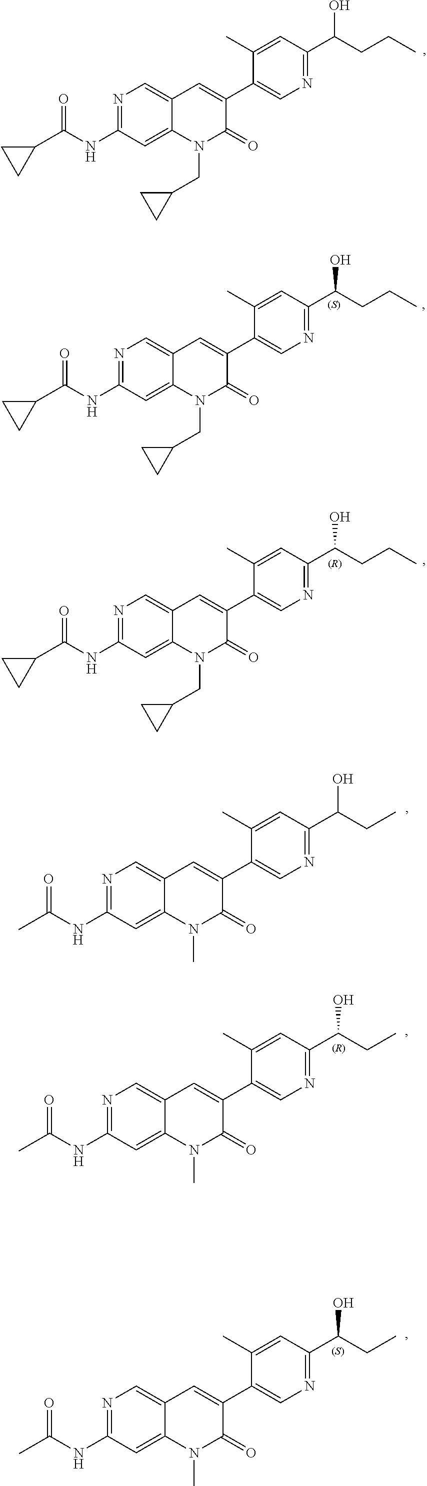

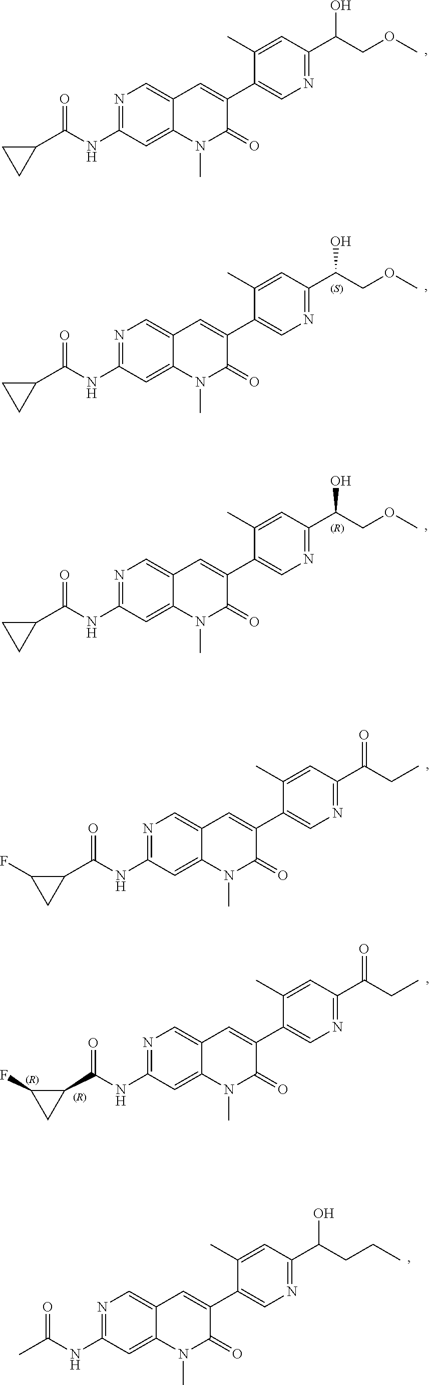

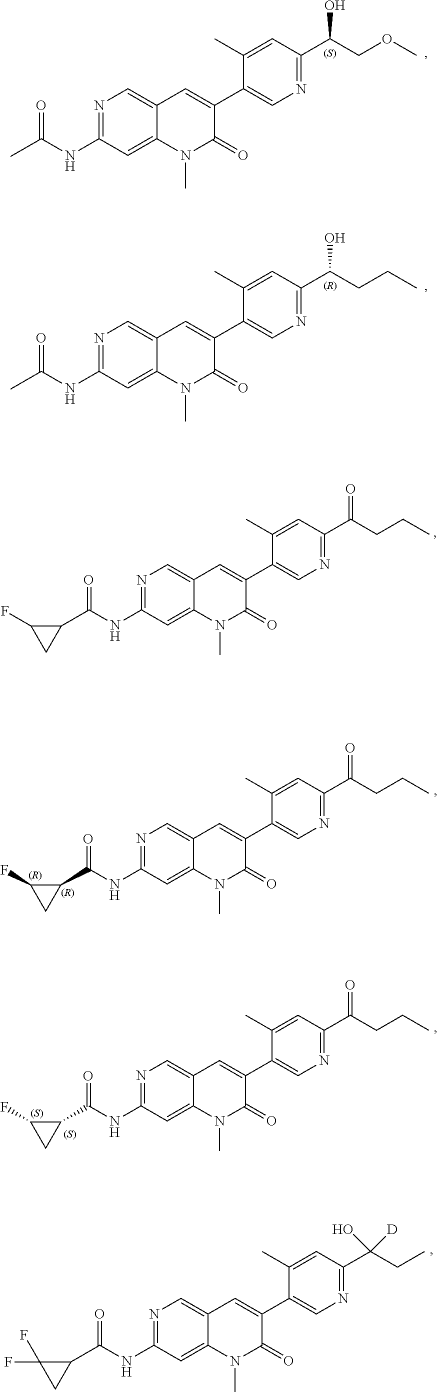

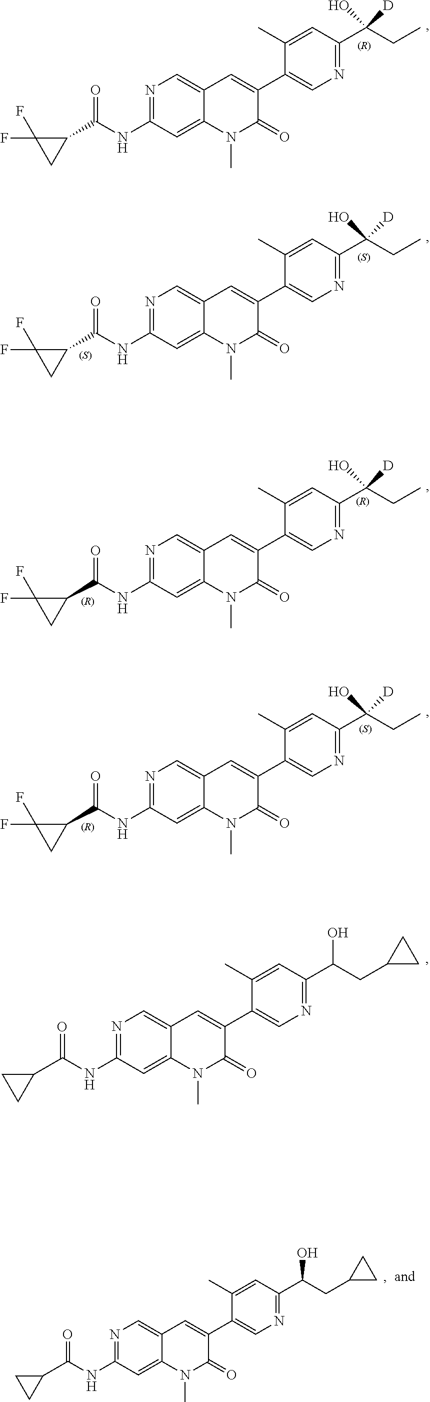

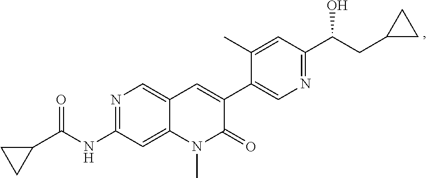 [SMILES: Cc1cc([C@H](O)CC2CC2)ncc1-c1cc2cnc(NC(=O)C3CC3)cc2n(C)c1=O]

or a pharmaceutically acceptable salt, solvate, hydrate, or co-crystal thereof, or a mixture of any of the foregoing.

Also provided herein is a compound of formula (I-a), (I-b), (I-c), (I-d), (I-e), (I-f), or (I-g):

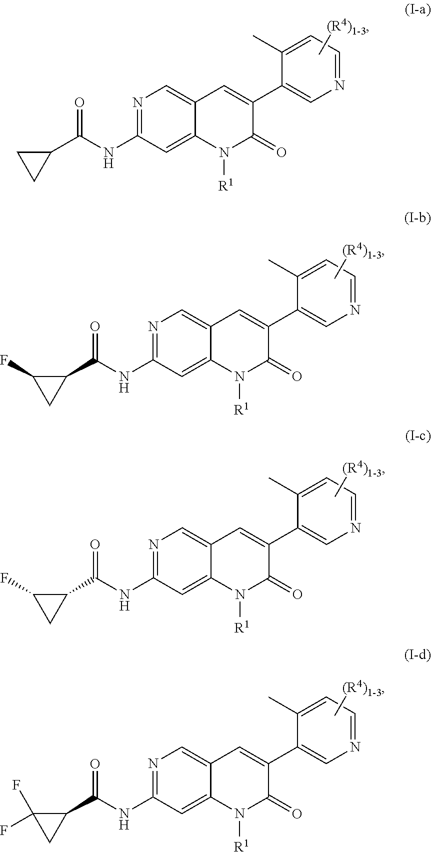

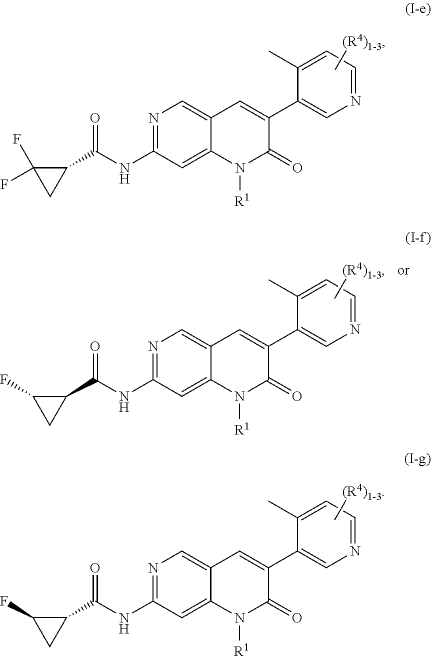

In some embodiments, R¹ is H or —CH₃; and each R⁴ is independently H, F, —CH(OH)CH₃, —CH(OH)CH₂CH₃, —CH(OH)CH₂CH₂CH₃, —CD(OH)CH₂CH₂CH₃, —C(CH₃)₂OH, or —CH(OH)CF₃. In some embodiments, R¹ is —CH₃; and each R⁴ is independently H, F, —CH(OH)CH₃, —C(CH₃)₂OH, or —CH(OH)CF₃.

In another aspect, provided herein is a pharmaceutical composition comprising any compound disclosed herein, or a pharmaceutically acceptable salt, solvate, hydrate, or co-crystal thereof, or a mixture of any of the foregoing, and one or more pharmaceutically acceptable excipients.

In yet another aspect, the present disclosure provides a method of inhibiting ARAF, BRAF and CRAF enzymatic activity in a cell, comprising exposing the cell with an effective amount of a compound of formula (I) as described herein, or a pharmaceutically acceptable salt, solvate, hydrate, or co-crystal thereof, or a mixture of any of the foregoing, or a pharmaceutical composition comprising a compound of formula (I) as described herein, or a pharmaceutically acceptable salt, solvate, hydrate, or co-crystal thereof, or a mixture of any of the foregoing.

In still yet another aspect, provided herein is a method of treating a cancer or neoplastic disease in a human in need thereof, comprising administering to the human a compound of formula (I) as described herein, or a pharmaceutically acceptable salt, solvate, hydrate, or co-crystal thereof, or a mixture of any of the foregoing, or a pharmaceutical composition comprising a compound of formula (I) as described herein, or a pharmaceutically acceptable salt, solvate, hydrate, or co-crystal thereof, or a mixture of any of the foregoing.

In still yet another aspect, provided herein is a method of treating a cancer or neoplastic disease in a human in need thereof, comprising administering to the human a compound of formula (I) as described herein, or a pharmaceutically acceptable salt, solvate, hydrate, or co-crystal thereof, or a mixture of any of the foregoing, or a pharmaceutical composition comprising a compound of formula (I) as described herein, or a pharmaceutically acceptable salt, solvate, hydrate, or co-crystal thereof, or a mixture of any of the foregoing, wherein the cancer or neoplastic disease is associated with one or more genetic alterations that engender elevated RAS/RAF/MEK/ERK pathway activation. In some embodiments, the cancer or neoplastic disease is associated with one or more genetic alterations in KRAS, NRAS, HRAS, ARAF, BRAF or CRAF. In some embodiments, the cancer or neoplastic disease is associated with one or more mutations in KRAS selected from the group consisting of G12D, G12V, G12C, G12S, G12R, G12A, G13D, G13C, G13R, Q61H, Q61K, Q61L, Q61P, Q61R and Q61E. In some embodiments, the cancer or neoplastic disease is associated with one or more mutations in NRAS selected from the group consisting of G12D, G12S, G12C, G12V, G12A, G13D, G13R, G13V, G13C, G13A, G13S, G61R, Q61K Q61H, and G61L. In some embodiments, the cancer or neoplastic disease is associated with one or more mutations in HRAS selected from the group consisting of G12V, G12S, G12D, G12C, G12R, G12A, G13R, G13V, G13D, G13S, G13C, Q61R, Q61L, Q61K, and Q61H. In some embodiments, the cancer or neoplastic disease is associated with one or more mutations in ARAF selected from the group consisting of S214C and S214F. In some embodiments, the cancer or neoplastic disease is associated with one or more mutations in BRAF selected from the group consisting of Class I, Class IIa, Class IIb, Class IIc, and Class III mutations. In some embodiments, the cancer or neoplastic disease is associated with one or more mutations in CRAF selected from the group consisting of P261A, P261L, E478K, R391W, R391S and T491I, or is associated with a CRAF fusion. In other embodiments, the cancer or neoplastic disease is associated with one or more genetic lesions resulting in the activation of one or more receptor tyrosine kinases (RTKs). In some embodiments, the one or more genetic lesions is a point mutation, a fusion or any combination thereof. In some embodiments, the one or more receptor tyrosine kinase is selected from the group consisting of ALK, EGFR, ERBB2, LTK, MET, NTRK, RET, and ROS1.

In some embodiments of the present aspect, the cancer is a refractory BRAF Class I mutant cancer. In some embodiments, the refractory BRAF Class I mutant cancer is associated with a point mutation selected from the group consisting of V600D, V600E, V600K, and V600R. In certain embodiments of the foregoing, the refractory cancer is associated with a genetic alteration in KRAS, NRAS, HRAS or BRAF that drives BRAF dimerization and confers resistance to approved Type 1.5 inhibitors (including vemurafenib, dabrafenib and encorafenib) both alone and in the context of MEK inhibitor (including cobimetinib, trametinib and binimetinib) combinations. In some embodiments, the refractory cancer is associated with one or more mutations in KRAS selected from the group consisting of G12D, G12V, G12C, G12S, G12R, G12A, G13D, G13C, G13R, Q61H, Q61K, Q61L, Q61P, Q61R and Q61E. In some embodiments, the refractory cancer is associated with one or more mutations in NRAS selected from the group consisting of G12D, G12S, G12C, G12V, G12A, G13D, G13R, G13V, G13C, G13A, G13S, G61R, Q61K Q61H, and G61L. In some embodiments, the refractory cancer is associated with one or more mutations in HRAS selected from the group consisting of G12V, G12S, G12D, G12C, G12R, G12A, G13R, G13V, G13D, G13S, G13C, Q61R, Q61L, Q61K, and Q61H. In some embodiments, the refractory cancer is associated with one or more genetic alterations in BRAF selected from the group consisting of gene amplification, point mutation, BRAF fusion, and gene splicing events. In some embodiments, the refractory cancer is associated with one or more Class II or Class III mutations in BRAF. In some embodiments, the refractory cancer is associated with one or more mutations in BRAF selected from the group consisting of G464V, G469A, G469V, G469R, E586K, K601E, K601N, G466R, G466A, G466E, G466V, N581I, N581S, D594E, D594G, D594N, G596C, G596R, L597R, L597S, and L597Q. In some embodiments, the refractory cancer is associated with one or more alternative splicing events that result in the loss of BRAF gene exons 4-10, 4-8, 2-8 or 2-10. In still further embodiments of the foregoing, the method further comprises administering one or more pharmaceutical agents including anti-microtubular therapies, topoisomerase inhibitors, alkylating agents, nucleotide synthesis inhibitors, DNA synthesis inhibitors, protein synthesis inhibitors, developmental signaling pathway inhibitors, pro-apoptotic agents, RTK inhibitors (including inhibitors against ALK, EGFR, ERBB2, LTK, MET, NTRK, RET, ROS1), RAF inhibitors representing alternative binding modes (such as Type 1.5 or Type II), MEK1/2 inhibitors, ERK1/2 inhibitors, RSK1/2/3/4 inhibitors, AKT inhibitors, TORC1/2 inhibitors, DNA damage response pathway inhibitors (including ATM, ATR), PI3K inhibitors and/or radiation.

In some embodiments of the present aspect, the cancer is a refractory cancer. In certain embodiments of the foregoing, the refractory cancer is associated with one or more genetic alterations in KRAS, NRAS, HRAS, BRAF, or one or more RTKs. In some embodiments, the refractory cancer is associated with one or more mutations in KRAS selected from the group consisting of G12D, G12V, G12C, G12S, G12R, G12A, G13D, G13C, G13R, Q61H, Q61K, Q61L, Q61P, Q61R and Q61E. In some embodiments, the refractory cancer is associated with one or more mutations in NRAS selected from the group consisting of G12D, G12S, G12C, G12V, G12A, G13D, G13R, G13V, G13C, G13A, G13S, G61R, Q61K Q61H, and G61L. In some embodiments, the refractory cancer is associated with one or more mutations in HRAS selected from the group consisting of G12V, G12S, G12D, G12C, G12R, G12A, G13R, G13V, G13D, G13S, G13C, Q61R, Q61L, Q61K, and Q61H. In some embodiments, the refractory cancer is associated with one or more genetic alterations in BRAF selected from the group consisting of gene amplification, point mutation, BRAF fusion, and gene splicing events. In some embodiments, the refractory cancer is associated with one or more Class II or Class III mutations in BRAF. In some embodiments, the refractory cancer is associated with one or more mutations in BRAF selected from the group consisting of G464V, G469A, G469V, G469R, E586K, K601E, K601N, G466R, G466A, G466E, G466V, N581I, N581S, D594E, D594G, D594N, G596C, G596R, L597R, L597S, and L597Q. In some embodiments, the refractory cancer is associated with one or more alternative splicing events that result in the loss of BRAF gene exons 4-10, 4-8, 2-8 or 2-10. In some embodiments wherein the refractory cancer is associated with one or more genetic alterations in one or more RTKs, the one or more RTKs is selected from the group consisting of ALK, EGFR, ERBB2, LTK, MET, NTRK, RET, and ROS1.

In some embodiments, the cancer is a solid tumor or a hematological malignancy. In some embodiments, the cancer is melanoma, lung cancer, pancreatic carcinoma, glioma, colorectal carcincoma, chronic myeloid leukemia (CML), acute myeloid leukemia (AML), or acute lymphoblastic leukemia (ALL). In certain embodiments, the lung cancer is non-small cell lung cancer (NSCLC).

In still further embodiments of the foregoing, the method further comprises administering one or more pharmaceutical agents including anti-microtubular therapies, topoisomerase inhibitors, alkylating agents, nucleotide synthesis inhibitors, DNA synthesis inhibitors, protein synthesis inhibitors, developmental signaling pathway inhibitors, pro-apoptotic agents, RTK inhibitors (including inhibitors against ALK, EGFR, ERBB2, LTK, MET, NTRK, RET, ROS1), RAF inhibitors representing alternative binding modes (such as Type 1.5 or Type II), MEK1/2 inhibitors, ERK1/2 inhibitors, RSK1/2/3/4 inhibitors, AKT inhibitors, TORC1/2 inhibitors, DNA damage response pathway inhibitors (including ATM, ATR), PI3K inhibitors and/or radiation.

In a further aspect, provided herein is a method of inhibiting Bcr-Abl enzymatic activity in a cell, comprising contacting the cell with an effective amount of any compound disclosed herein, or a pharmaceutically acceptable salt, solvate, hydrate, or co-crystal thereof, or a mixture of any of the foregoing, or the pharmaceutical composition disclosed herein.

In another aspect, provided herein is a method of treating chronic myeloid leukemia (CML), acute myeloid leukemia (AML), or acute lymphoblastic leukemia (ALL) in a human in need thereof, comprising administering to the human any compound disclosed herein, or a pharmaceutically acceptable salt, solvate, hydrate, or co-crystal thereof, or a mixture of any of the foregoing, or the pharmaceutical composition disclosed herein.

In some embodiments, the chronic myeloid leukemia is refractory chronic myeloid leukemia. In some embodiments, the refractory chronic myeloid leukemia is associated with a mutation selected from the group consisting of M244V, L248V, G250E, G250A, Q252H, Q252R, Y253F, Y253H, E255K, E255V, D276G, F311L, T315N, T315A, F317V, F317L, M343T, M351T, E355G, F359A, F359V, V379I, F382L, L387M, H396P, H396R, S417Y, E459K, F486S, and T315I.

### DETAILED DESCRIPTION

The following description sets forth exemplary methods, parameters, and the like. It should be recognized, however, that such description is not intended as a limitation on the scope of the present disclosure but is instead provided as a description of exemplary embodiments.

### I. Definitions

As used herein, the following definitions shall apply unless otherwise indicated. Further, if any term or symbol used herein is not defined as set forth below, it shall have its ordinary meaning in the art.

The term “excipient” as used herein means an inert or inactive substance that may be used in the production of a drug or pharmaceutical, such as a tablet containing a compound of the present disclosure as an active ingredient. Various substances may be embraced by the term excipient, including without limitation any substance used as a binder, disintegrant, coating, compression/encapsulation aid, cream or lotion, lubricant, solutions for parenteral administration, materials for chewable tablets, sweetener or flavoring, suspending/gelling agent, or wet granulation agent. Binders include, e.g., carbomers, povidone, xanthan gum, etc.; coatings include, e.g., cellulose acetate phthalate, ethylcellulose, gellan gum, maltodextrin, enteric coatings, etc.; compression/encapsulation aids include, e.g., calcium carbonate, dextrose, fructose dc (dc=“directly compressible”), honey dc, lactose (anhydrate or monohydrate; optionally in combination with aspartame, cellulose, or microcrystalline cellulose), starch dc, sucrose, etc.; disintegrants include, e.g., croscarmellose sodium, gellan gum, sodium starch glycolate, etc.; creams or lotions include, e.g., maltodextrin, carrageenans, etc.; lubricants include, e.g., magnesium stearate, stearic acid, sodium stearyl fumarate, etc.; materials for chewable tablets include, e.g., dextrose, fructose dc, lactose (monohydrate, optionally in combination with aspartame or cellulose), etc.; suspending/gelling agents include, e.g., carrageenan, sodium starch glycolate, xanthan gum, etc.; sweeteners include, e.g., aspartame, dextrose, fructose dc, sorbitol, sucrose dc, etc.; and wet granulation agents include, e.g., calcium carbonate, maltodextrin, microcrystalline cellulose, etc.

The terms “individual”, “subject” and “patient” refer to mammals and includes humans and non-human mammals. Examples of patients include, but are not limited to, mice, rats, hamsters, guinea pigs, pigs, rabbits, cats, dogs, goats, sheep, cows, and humans. In some embodiments, patient refers to a human.

As used herein, the term “mammal” includes, but is not limited to, humans, mice, rats, guinea pigs, monkeys, dogs, cats, horses, cows, pigs, and sheep.

“Pharmaceutically acceptable” refers to safe and non-toxic, and suitable for in vivo or for human administration.

As used herein, the term “alkyl”, by itself or as part of another substituent, means, unless otherwise stated, a straight or branched chain hydrocarbon radical, having the number of carbon atoms designated (e.g., C₁-C₆ means one to six carbons). Examples of alkyl groups include methyl, ethyl, n-propyl, iso-propyl, n-butyl, t-butyl, iso-butyl, sec-butyl, n-pentyl, n-hexyl, n-heptyl, n-octyl, and the like. In some embodiments, the term “alkyl” may encompass C₁-C₆ alkyl, C₂-C₆ alkyl, C₃-C₆ alkyl, C₄-C₆ alkyl, C₅-C₆ alkyl, C₁-C₅ alkyl, C₂-C₅ alkyl, C₃-C₅ alkyl, C₄-C₅ alkyl, C₁-C₄ alkyl, C₂-C₄ alkyl, C₃-C₄ alkyl, C₁-C₃ alkyl, C₂-C₃ alkyl, or C₁-C₂ alkyl.

The term “cycloalkyl,” “carbocyclic,” or “carbocycle” refers to hydrocarbon rings having the indicated number of ring atoms (e.g., C₃-C₆ cycloalkyl means 3-6 carbons) and being fully saturated or having no more than one double bond between ring vertices. As used herein, “cycloalkyl,” “carbocyclic,” or “carbocycle” is also meant to refer to bicyclic, polycyclic and spirocyclic hydrocarbon rings such as, for example, bicyclo[2.2.1]heptane, pinane, bicyclo[2.2.2]octane, adamantane, norborene, spirocyclic C₅₋₁₂ alkane, etc. In some embodiments, “cycloalkyl” encompasses C₃-C₇ cycloalkyl, C₄-C₇ cycloalkyl, C₅-C₇ cycloalkyl, C₅-C₇ cycloalkyl, C₃-C₆ cycloalkyl, C₄-C₆ cycloalkyl, C₅-C₆ cycloalkyl, C₃-C₅ cycloalkyl, C₄-C₅ cycloalkyl, or C₃-C₄ cycloalkyl.

The term “heteroalkyl,” by itself or in combination with another term, means, unless otherwise stated, a stable straight or branched chain hydrocarbon radical, consisting of the stated number of carbon atoms and from one to three heteroatoms selected from the group consisting of O, N, Si and S, and wherein the nitrogen and sulfur atoms can optionally be oxidized and the nitrogen heteroatom can optionally be quaternized. The heteroatom(s) 0, N and S can be placed at any interior position of the heteroalkyl group. The heteroatom Si can be placed at any position of the heteroalkyl group, including the position at which the alkyl group is attached to the remainder of the molecule. A “heteroalkyl” can contain up to three units of unsaturation, and also include mono- and poly-halogenated variants, or combinations thereof. Examples include
—CH₂—CH₂—O—CH₃, —CH₂—CH₂—O—CF₃, —CH₂—CH₂—NH—CH₃, —CH₂—CH₂—N(CH₃)—CH₃, —CH₂—S—CH₂—CH₃, —S(O)—CH₃, —CH₂—CH₂—S(O)₂—CH₃, —CH═CH—O—CH₃, —Si(CH₃)₃, —CH₂—CH═N—OCH₃, and —CH═CH═N(CH₃)—CH₃. Up to two heteroatoms can be consecutive, such as, for example, —CH₂—NH—OCH₃ and —CH₂—O—Si(CH₃)₃.

The term “heterocycloalkyl,” “heterocyclic,” or “heterocycle” refers to a cycloalkyl radical group having the indicated number of ring atoms (e.g., 5-6 membered heterocycloalkyl) that contain from one to five heteroatoms selected from the group consisting of N, O, and S, wherein the nitrogen and sulfur atoms are optionally oxidized, nitrogen atom(s) are optionally quaternized, as ring atoms. Unless otherwise stated, a “heterocycloalkyl,” “heterocyclic,” or “heterocycle” ring can be a monocyclic, a bicyclic, spirocyclic or a polycylic ring system. Non-limiting examples of “heterocycloalkyl,” “heterocyclic,” or “heterocycle” rings include pyrrolidine, piperidine, N-methylpiperidine, imidazolidine, pyrazolidine, butyrolactam, valerolactam, imidazolidinone, hydantoin, dioxolane, phthalimide, piperidine, pyrimidine-2,4(1H,3H)-dione, 1,4-dioxane, morpholine, thiomorpholine, thiomorpholine-5-oxide, thiomorpholine-S,S-oxide, piperazine, pyran, pyridone, 3-pyrroline, thiopyran, pyrone, tetrahydrofuran, tetrhydrothiophene, quinuclidine, tropane and the like. A “heterocycloalkyl,” “heterocyclic,” or “heterocycle” group can be attached to the remainder of the molecule through one or more ring carbons or heteroatoms. In some embodiments, “heterocycloalkyl” encompasses 4- to 8-membered heterocycloalkyl, 5- to 8-membered heterocycloalkyl, 6- to 8-membered heterocycloalkyl, 7- to 8-membered heterocycloalkyl, 4- to 7-membered heterocycloalkyl, 5- to 7-membered heterocycloalkyl, 6- to 7-membered heterocycloalkyl, 4- to 6-membered heterocycloalkyl, 5- to 6-membered heterocycloalkyl, or 4- to 5-membered heterocycloalkyl.

The term “alkylene” by itself or as part of another substituent means a divalent radical derived from an alkane, as exemplified by —CH₂CH₂CH₂CH₂—. Typically, an alkyl (or alkylene) group will have from 1 to 24 carbon atoms. In some embodiments, an alkyl (or alkylene) group will have 10 or fewer carbon atoms.

The term “heteroalkylene” by itself or as part of another substituent means a divalent radical, saturated or unsaturated or polyunsaturated, derived from heteroalkyl, as exemplified by —CH₂—CH₂—S—CH₂CH₂—, —CH₂—S—CH₂—CH₂—NH—CH₂—, —O—CH₂—CH═CH—, —CH₂—CH═C(H)CH₂—O—CH₂— and —S—CH₂—C≡C—. For heteroalkylene groups, heteroatoms can also occupy either or both of the chain termini (e.g., alkyleneoxy, alkylenedioxy, alkyleneamino, alkylenediamino, and the like).

The term “heterocycloalkylene” by itself or as part of another substituent means a divalent radical, saturated or unsaturated or polyunsaturated, derived from heterocycloalkyl. For heterocycloalkylene groups, heteroatoms can also occupy either or both of the chain termini.

The terms “alkoxy” and “alkylamino” are used in their conventional sense, and refer to those alkyl groups attached to the remainder of the molecule via an oxygen atom or an amino group, respectively.

The term “heterocycloalkoxy” refers to a heterocycloalkyl-O— group in which the heterocycloalkyl group is as previously described herein.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “C₁-C₄ haloalkyl” is mean to include trifluoromethyl, 2,2,2-trifluoroethyl, 4-chlorobutyl, 3-bromopropyl, difluoromethyl, and the like.

The term “haloalkyl-OH” refers to a haloalkyl group as described above which is also substituted by one or more hydroxyl groups. The term “haloalkyl-OH” is meant to include haloalkyl substituted by one hydroxyl group, as well as haloalkyl substituted by multiple hydroxyl groups. The term “haloalkyl-OH” also encompasses haloalkyl groups substituted by one or more hydroxyl groups on any carbon of the haloalkyl group. For example, the term “haloalkyl-OH” includes —CH(F)OH, —CH₂CFHCH₂OH, —CH(OH)CF₃, and the like.

The term “alkyl-OH” or “alkylene-OH” refers to an alkyl or alkylene substituted by one or more hydroxyl groups. The term “alkyl-OH” is meant to include alkyl substituted by one hydroxyl group, as well as alkyl substituted by multiple hydroxyl groups. The term “alkylene-OH” is meant to include alkylene substituted by one hydroxyl group, as well as alkylene substituted by multiple hydroxyl groups. The terms “alkyl-OH” and “alkylene-OH” also encompass alkyl groups and alkylene groups, respectively, that are substituted by one or more hydroxyl groups on any carbon of the alkyl or alkylene group, as valency permits. For example, the term “alkyl-OH” includes —CH₂OH, —CH(OH)CH₃, —CH₂CH₂OH, —C(CH₃)₂OH, and the like.

The term “alkyl-OR⁶” refers to an alkyl substituted by one or more —OR⁶ groups. The term “alkyl-OR⁶” is meant to include alkyl substituted by one —OR⁶ group, as well as alkyl substituted by multiple —OR⁶ groups. The term “alkyl-OR⁶” also encompasses alkyl groups substituted by one or more OR⁶ groups on any carbon of the alkyl, as valency permits.

The term “alkyl-CN” refers to an alkyl substituted by one or more cyano groups. The term “alkyl-CN” is meant to include alkyl substituted by one cyano group, as well as alkyl substituted by multiple cyano groups. The term “alkyl-CN” also encompasses alkyl groups substituted by one or more cyano groups on any carbon of the alkyl group. For example, the term “alkyl-CN” includes —CH₂CN, —CH₂CH₂CN, —CH(CN)CH₃, and the like.

The term “aryl” means, unless otherwise stated, a polyunsaturated, typically aromatic, hydrocarbon group, which can be a single ring or multiple rings (up to three rings) which are fused together. In some embodiments, “aryl” encompasses C₆-C₁₄ aryl, C₅-C₁₄ aryl, C₁₀-C₁₄ aryl, C₁₂-C₁₄ aryl, C₆-C₁₂ aryl, C₅-C₁₂ aryl, C₁₀-C₁₂ aryl, C₆-C₁₀ aryl, C₅-C₁₀ aryl, or C₆-C₈ aryl. The term “heteroaryl” refers to aryl groups (or rings) that contain from one to five heteroatoms selected from the group consisting of N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl groups include phenyl, naphthyl and biphenyl, while non-limiting examples of heteroaryl groups include pyridyl, pyridazinyl, pyrazinyl, pyrimindinyl, triazinyl, quinolinyl, quinoxalinyl, quinazolinyl, cinnolinyl, phthalaziniyl, benzotriazinyl, purinyl, benzimidazolyl, benzopyrazolyl, benzotriazolyl, benzisoxazolyl, isobenzofuryl, isoindolyl, indolizinyl, benzotriazinyl, thienopyridinyl, thienopyrimidinyl, pyrazolopyrimidinyl, imidazopyridines, benzothiaxolyl, benzofuranyl, benzothienyl, indolyl, quinolyl, isoquinolyl, isothiazolyl, pyrazolyl, indazolyl, pteridinyl, imidazolyl, triazolyl, tetrazolyl, oxazolyl, isoxazolyl, thiadiazolyl, pyrrolyl, thiazolyl, furyl, thienyl and the like. In some embodiments, the term “heteroaryl” encompasses 5- to 10-membered heteroaryl, 6- to 10-membered heteroaryl, 7- to 10-membered heteroaryl, 8- to 10-membered heteroaryl, 9- to 10-membered heteroaryl, 5- to 9-membered heteroaryl, 6- to 9-membered heteroaryl, 7- to 9-membered heteroaryl, 8- to 9-membered heteroaryl, 5- to 8-membered heteroaryl, 6- to 8-membered heteroaryl, 7- to 8-membered heteroaryl, 5- to 7-membered heteroaryl, 6- to 7-membered heteroaryl, or 5- to 6-membered heteroaryl.

The above terms (e.g., “alkyl,” “aryl” and “heteroaryl”), in some embodiments, will include both substituted and unsubstituted forms of the indicated radical.

As used herein, the term “heteroatom” is meant to include oxygen (O), nitrogen (N), sulfur (S) and silicon (Si).

As used herein, the term “chiral” refers to molecules which have the property of non-superimposability of the mirror image partner, while the term “achiral” refers to molecules which are superimposable on their mirror image partner.

As used herein, the term “stereoisomers” refers to compounds which have identical chemical constitution, but differ with regard to the arrangement of the atoms or groups in space.

As used herein, a wavy line “ [symbol] ” that intersects a bond in a chemical structure indicates the point of attachment of the atom to which the wavy bond is connected in the chemical structure to the remainder of a molecule, or to the remainder of a fragment of a molecule.

As used herein, the representation of a group (e.g., X^{a}) in parenthesis followed by a subscript integer range (e.g., (X^{a})₀₋₁) means that the group can have the number of occurrences as designated by the integer range. For example, (X^{a})₀₋₁ means the group X^{a} can be absent or can occur one time.

“Diastereomer” refers to a stereoisomer with two or more centers of chirality and whose molecules are not mirror images of one another. Diastereomers have different physical properties, e.g. melting points, boiling points, spectral properties, and reactivities. Mixtures of diastereomers can separate under high resolution analytical procedures such as electrophoresis and chromatography.

“Enantiomers” refer to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the present disclosure can contain asymmetric or chiral centers, and therefore exist in different stereoisomeric forms. It is intended that all stereoisomeric forms of the compounds of the present disclosure, including but not limited to, diastereomers, enantiomers and atropisomers, as well as mixtures thereof such as racemic mixtures, form part of the present disclosure. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer can also be referred to as an enantiomer, and a mixture of such isomers is often called an enantiomeric mixture. A 50:50 mixture of enantiomers is referred to as a racemic mixture or a racemate, which can occur where there has been no stereoselection or stereospecificity in a chemical reaction or process. The terms “racemic mixture” and “racemate” refer to an equimolar mixture of two enantiomeric species, devoid of optical activity.

As used herein, the term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

As used herein, the term “solvate” refers to an association or complex of one or more solvent molecules and a compound of the present disclosure. Examples of solvents that form solvates include, but are not limited to, water, isopropanol, ethanol, methanol, DMSO, ethyl acetate, acetic acid, and ethanolamine. The term “hydrate” refers to the complex where the solvent molecule is water. Certain compounds of the present disclosure can exist in unsolvated forms as well as solvated forms, including hydrated forms. In general, the solvated forms are equivalent to unsolvated forms and are intended to be encompassed within the scope of the present disclosure.

The term “co-crystal” as used herein refers to a solid that is a crystalline single phase material composed of two or more different molecular or ionic compounds generally in a stoichiometric ratio which are neither solvates nor simple salts. A co-crystal consists of two or more components that form a unique crystalline structure having unique properties. Co-crystals are typically characterized by a crystalline structure, which is generally held together by freely reversible, non-covalent interactions. As used herein, a co-crystal refers to a compound of the present disclosure and at least one other component in a defined stoichiometric ratio that form a crystalline structure.

As used herein, the term “protecting group” refers to a substituent that is commonly employed to block or protect a particular functional group on a compound. For example, an “amino-protecting group” is a substituent attached to an amino group that blocks or protects the amino functionality in the compound. Suitable amino-protecting groups include acetyl, trifluoroacetyl, t-butoxycarbonyl (BOC), benzyloxycarbonyl (CBZ) and 9-fluorenylmethylenoxycarbonyl (Fmoc). Similarly, a “hydroxy-protecting group” refers to a substituent of a hydroxy group that blocks or protects the hydroxy functionality. Suitable protecting groups include acetyl and silyl. A “carboxy-protecting group” refers to a substituent of the carboxy group that blocks or protects the carboxy functionality. Common carboxy-protecting groups include phenylsulfonylethyl, cyanoethyl, 2-(trimethylsilyl)ethyl, 2-(trimethylsilyl)ethoxymethyl, 2-(p-toluenesulfonyl)ethyl, 2-(p-nitrophenylsulfenyl)ethyl, 2-(diphenylphosphino)-ethyl, nitroethyl and the like. For a general description of protecting groups and their use, see P. G. M. Wuts and T. W. Greene, Greene's Protective Groups in Organic Synthesis 4^{th} edition, Wiley-Interscience, New York, 2006.

As used herein, the term “pharmaceutically acceptable salts” is meant to include salts of the active compounds which are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of salts derived from pharmaceutically-acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

The neutral forms of the compounds can be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present disclosure.

Certain compounds of the present disclosure possess asymmetric carbon atoms (optical centers) or double bonds; the racemates, diastereomers, geometric isomers, regioisomers and individual isomers (e.g., separate enantiomers) are all intended to be encompassed within the scope of the present disclosure.

The compounds of the present disclosure can also contain unnatural proportions of atomic isotopes at one or more of the atoms that constitute such compounds. For example, the present disclosure also embraces isotopically-labeled variants of the present disclosure which are identical to those recited herein, but for the fact that one or more atoms are replaced by an atom having the atomic mass or mass number different from the predominant atomic mass or mass number usually found in nature for the atom. All isotopes of any particular atom or element as specified are contemplated within the scope of the compounds of the present disclosure and include isotopes of hydrogen, carbon, nitrogen, oxygen, phosphorous, sulfur, fluorine, chlorine and iodine, such as ²H (“D”), ³H, ¹¹C, ¹³C, ¹⁴C, ¹³N, ¹⁵N, ¹⁵O, ¹⁷O, ¹⁸O, ³²P, ³³P, 35S, ¹⁸F, ³⁶Cl, ¹²³I and ¹²⁵I. Certain isotopically labeled compounds of the present disclosure (e.g., those labeled with ³H or ¹⁴C) are useful in compound and/or substrate tissue distribution assays. Tritiated (³H) and carbon-14 (¹⁴C) isotopes are useful for their ease of preparation and detectability. Further substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, ¹¹C, and ¹⁸F are useful for positron emission tomography (PET) studies to examine substrate receptor occupancy. Isotopically labeled compounds of the present disclosure can generally be prepared by following procedures analogous to those disclosed in the Schemes and/or in the Examples herein below, by substituting an isotopically labeled reagent for a non-isotopically labeled reagent.

“Treating” or “treatment” of a disease in a patient refers to inhibiting the disease or arresting its development; or ameliorating or causing regression of the disease. As used herein, “treatment” or “treating” is an approach for obtaining beneficial or desired results including clinical results. For purposes of this disclosure, beneficial or desired results include, but are not limited to, one or more of the following: decreasing one more symptoms resulting from the disease or disorder, diminishing the extent of the disease or disorder, stabilizing the disease or disorder (e.g., preventing or delaying the worsening of the disease or disorder), delaying the occurrence or recurrence of the disease or disorder, delay or slowing the progression of the disease or disorder, ameliorating the disease or disorder state, providing a remission (whether partial or total) of the disease or disorder, decreasing the dose of one or more other medications required to treat the disease or disorder, enhancing the effect of another medication used to treat the disease or disorder, delaying the progression of the disease or disorder, increasing the quality of life, and/or prolonging survival of a patient. Also encompassed by “treatment” is a reduction of pathological consequence of the disease or disorder. The methods of the present disclosure contemplate any one or more of these aspects of treatment.

“Preventing”, “prevention”, or “prophylaxis” of a disease in a patient refers to preventing the disease from occurring in a patient that is predisposed or does not yet display symptoms of the disease.

The phrase “therapeutically effective amount” means an amount of a compound of the present disclosure that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The terms “cancer” and “cancerous” refer to or describe the physiological condition in mammals that is typically characterized by unregulated cell growth.

It is appreciated that certain features of the present disclosure, which are, for clarity, described in the context of separate embodiments, may also be provided in combination in a single embodiment. Conversely, various features of the invention, which are, for brevity, described in the context of a single embodiment, may also be provided separately or in any suitable subcombination. All combinations of the embodiments pertaining to the chemical groups represented by the variables are specifically embraced by the present invention and are disclosed herein just as if each and every combination was individually and explicitly disclosed, to the extent that such combinations embrace compounds that are stable compounds (i.e., compounds that can be isolated, characterized, and tested for biological activity). In addition, all subcombinations of the chemical groups listed in the embodiments describing such variables are also specifically embraced by the present invention and are disclosed herein just as if each and every such sub-combination of chemical groups was individually and explicitly disclosed herein.

### II. Compounds

In one aspect, provided herein is a compound of formula (I)

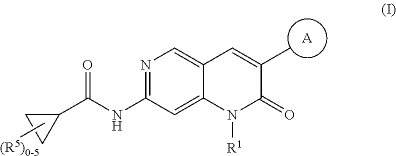

or a pharmaceutically acceptable salt, solvate, hydrate, or co-crystal thereof, or a mixture of any of the foregoing, wherein:
R¹ is H, C₁-C₆ alkyl, C₁-C₆ haloalkyl, C₁-C₆ heteroalkyl, C₃-C₇ cycloalkyl, C₁-C₆ alkyl-CN, 4- to 7-membered heterocycloalkyl, —(X^{a})₀₋₁—(C(R²)R²)₀₋₁—OR²,
   —(X^{a})₀₋₁—(C(R²)R²)₀₋₁—N(R²)R², —(X^{a})₀₋₁—(C(R²)R²)₀₋₁—S(O)₂R²,
   —(X^{a})—(C(R²)R²)₀₋₁—S(O)(═NR²)R², —(X^{a})—C(R²)R²—S(O)₂N(R²)R²,
   —(X^{a})—C(R²)R²—S(O)R², —(X^{a})—C(R²)R²—N(R²)R²S(O)₂R², or
   —(X^{a})—(C(R²)R²)₀₋₁—C(O)N(R²)R²,
   wherein the aliphatic portions of R¹ are optionally substituted with 1 to 5 R³ groups;
X^{a} is C₁-C₆ alkylene, C₁-C₆ heteroalkylene, C₃-C₇ cycloalkylene, or C₄-C₇ heterocycloalkylene, each of which is optionally substituted with 1 to 5 R³ groups;
each R² is independently H, C₁-C₆ alkyl, C₁-C₆ haloalkyl, C₁-C₆ heteroalkyl, C₃-C₇ cycloalkyl, 4- to 7-membered heterocycloalkyl, or 5- to 6-membered heteroaryl,
   wherein the aliphatic and aromatic portions of R² are optionally substituted with 1 to 5 R³ groups, and
   wherein any two R² groups attached to the same nitrogen are optionally taken together to form a 4- to 7-membered heterocyclic ring, or any two R² groups attached to the same carbon atom are optionally taken together to form a 3- to 6-membered carbocyclic ring or a 4- to 6-membered heterocyclic ring optionally containing 1-3 heteroatoms selected from the group consisting of N, O, and S, wherein each carbocyclic or heterocyclic ring is optionally substituted with 1 to 5 R³ groups;
each R³ is independently C₁-C₆ alkyl, C₁-C₆-alkoxy, C₃-C₇ cycloalkyl, C₃-C₇ cycloalkoxy,
   C₁-C₆ haloalkyl, C₁-C₆ alkylamino, C₁-C₆ dialkylamino, 4- to 7-membered heterocycloalkyl, 4- to 7-membered heterocycloalkoxy, 5- to 6-membered heteroaryl, F, Cl, —OH, —NH₂, —NHMe, —NMe₂, —SH, —SMe, —S(O)Me, —S(O)₂Me,
   —S(O)(═NH)Me, —S(O)(═NMe)Me, —CN, —NO₂, —CHF₂, —CF₃, —CF₂Cl, —OCF₃, —OCHF₂, —SCF₃, —SCHF₂, —SF₅, —P(O)(Me)₂, or —N₃;
Ring A is C₆-C₁₀ aryl or 5- to 10-membered heteroaryl,
   wherein the aromatic portions of Ring A are optionally substituted with 1 to 5 R⁴ groups, and
   wherein when Ring A is C₆ aryl or 5- to 6-membered heteroaryl, then any two substituents attached to adjacent atoms of said aryl or heteroaryl are optionally taken together to form a 4- to 6-membered carbocyclic ring or a 4- to 6-membered heterocyclic ring optionally containing 1-3 heteroatoms selected from the group consisting of N, O, and S, wherein each carbocyclic or heterocyclic ring is optionally substituted with 1 to 5 R⁴ groups;
each R⁴ is independently F, Cl, Br, I, —SCF₃, —SCHF₂, —SF₅, —OCF₃, —OR⁶, —N(R⁶)R⁶, —N₃, —OCHF₂, —CF₃, —CF₂Cl, —CHF₂, —CN, —NO₂, —C(═O)C₁-C₆ alkyl, C₁-C₆ alkyl, C₁-C₆ haloalkyl, C₁-C₆ haloalkyl-OH, C₁-C₆ alkyl-OH, C₁-C₆ alkyl-OR⁶, C₁-C₆ alkylene-C₁-C₃ alkoxy, C₁-C₆ alkylene(—OH)—C₁-C₃ alkoxy, C₁-C₆ alkyl-CN, C₁-C₆ heteroalkyl, C₃-C₇ cycloalkyl, C₁-C₆ alkylene-C₃-C₇ cycloalkyl, C₁-C₆ alkylene(—OH)—C₃-C₇ cycloalkyl, or 5- to 6-membered heteroaryl;
each R⁵ is independently F, Cl, —SCF₃, —SCHF₂, —SF₅, —OCF₃, —OR⁶, —N(R⁶)R⁶, —N₃, —OCHF₂, —CF₃, —CF₂Cl, —CHF₂, —CN, —NO₂, C₁-C₆ alkyl, C₁-C₆ haloalkyl, C₁-C₆ heteroalkyl, or C₃-C₇ cycloalkyl,
   wherein the aliphatic portions of R⁵ are optionally substituted with 1 to 3 R³ groups; and
each R⁶ is independently H, C₁-C₆ alkyl, C₁-C₆ haloalkyl, C₁-C₆ heteroalkyl, C₃-C₇ cycloalkyl, C₆ aryl, 5- to 10-membered heteroaryl, or 4- to 7-membered heterocycloalkyl.

In some embodiments, the compound of formula (I) is a compound of formula (I-1),

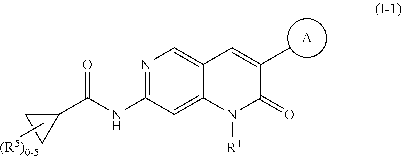

or a pharmaceutically acceptable salt, solvate, hydrate, or co-crystal thereof, or a mixture of any of the foregoing.

In other embodiments, the compound of formula (I) is a compound of formula (I-2):

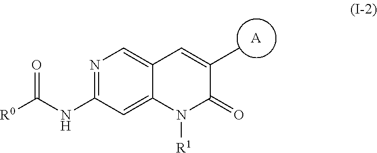

or a pharmaceutically acceptable salt, solvate, hydrate, or co-crystal thereof, or a mixture of any of the foregoing, wherein R⁰ is C₁-C₃ alkyl optionally substituted by 1 to 5 R⁵ groups.

In one aspect, provided is a compound of formula (I-1)

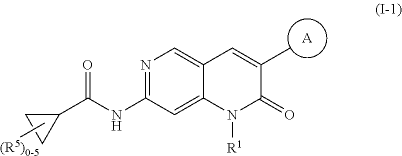

or a pharmaceutically acceptable salt, solvate, hydrate, or co-crystal thereof, or a mixture of any of the foregoing, wherein:
R¹ is H, C₁-C₆ alkyl, C₁-C₆ haloalkyl, C₁-C₆ heteroalkyl, C₃-C₇ cycloalkyl, C₁-C₆ alkyl-CN, 4- to 7-membered heterocycloalkyl, —(X^{a})₀₋₁—(C(R²)R²)₀₋₁—OR²,
   —(X^{a})₀₋₁—(C(R²)R²)₀₋₁—N(R²)R², —(X^{a})₀₋₁—(C(R²)R²)₀₋₁—S(O)₂R²,
   —(X^{a})—(C(R²)R²)₀₋₁—S(O)(═NR²)R², —(X^{a})—C(R²)R²—S(O)₂N(R²)R²,
   —(X^{a})—C(R²)R²—S(O)R², —(X^{a})—C(R²)R²—N(R²)R²S(O)₂R², or
   —(X^{a})—(C(R²)R²)₀₋₁—C(O)N(R²)R²,
   wherein the aliphatic portions of R¹ are optionally substituted with 1 to 5 R³ groups;
X^{a} is C₁-C₆ alkylene, C₁-C₆ heteroalkylene, C₃-C₇ cycloalkylene, or C₄-C₇ heterocycloalkylene, each of which is optionally substituted with 1 to 5 R³ groups;
each R² is independently H, C₁-C₆ alkyl, C₁-C₆ haloalkyl, C₁-C₆ heteroalkyl, C₃-C₇ cycloalkyl, 4- to 7-membered heterocycloalkyl, or 5- to 6-membered heteroaryl,
   wherein the aliphatic and aromatic portions of R² are optionally substituted with 1 to 5 R³ groups, and
   wherein any two R² groups attached to the same nitrogen are optionally taken together to form a 4- to 7-membered heterocyclic ring, or any two R² groups attached to the same carbon atom are optionally taken together to form a 3- to 6-membered carbocyclic ring or a 4- to 6-membered heterocyclic ring optionally containing 1-3 heteroatoms selected from the group consisting of N, O, and S, wherein each carbocyclic or heterocyclic ring is optionally substituted with 1 to 5 R³ groups;
each R³ is independently C₁-C₆ alkyl, C₁-C₆-alkoxy, C₃-C₇ cycloalkyl, C₃-C₇ cycloalkoxy,
   C₁-C₆ haloalkyl, C₁-C₆ alkylamino, C₁-C₆ dialkylamino, 4- to 7-membered heterocycloalkyl, 4- to 7-membered heterocycloalkoxy, 5- to 6-membered heteroaryl, F, Cl, —OH, —NH₂, —NHMe, —NMe₂, —SH, —SMe, —S(O)Me, —S(O)₂Me,
   —S(O)(═NH)Me, —S(O)(═NMe)Me, —CN, —NO₂, —CHF₂, —CF₃, —CF₂Cl, —OCF₃, —OCHF₂, —SCF₃, —SCHF₂, —SF₅, —P(O)(Me)₂, or —N₃;
Ring A is C₆-C₁₀ aryl or 5- to 10-membered heteroaryl,
   wherein the aromatic portions of Ring A are optionally substituted with 1 to 5 R⁴ groups, and
   wherein when Ring A is C₆ aryl or 5- to 6-membered heteroaryl, then any two substituents attached to adjacent atoms of said aryl or heteroaryl are optionally taken together to form a 4- to 6-membered carbocyclic ring or a 4- to 6-membered heterocyclic ring optionally containing 1-3 heteroatoms selected from the group consisting of N, O, and S, wherein each carbocyclic or heterocyclic ring is optionally substituted with 1 to 5 R⁴ groups;
each R⁴ is independently F, Cl, Br, I, —SCF₃, —SCHF₂, —SF₅, —OCF₃, —OR⁶, —N(R⁶)R⁶, —N₃, —OCHF₂, —CF₃, —CF₂Cl, —CHF₂, —CN, —NO₂, —C(═O)C₁-C₆ alkyl, C₁-C₆ alkyl, C₁-C₆ haloalkyl, C₁-C₆ haloalkyl-OH, C₁-C₆ alkyl-OH, C₁-C₆ alkyl-OR⁶, C₁-C₆ alkylene-C₁-C₃ alkoxy, C₁-C₆ alkylene(—OH)—C₁-C₃ alkoxy, C₁-C₆ alkyl-CN, C₁-C₆ heteroalkyl, C₃-C₇ cycloalkyl, C₁-C₆ alkylene-C₃-C₇ cycloalkyl, C₁-C₆ alkylene(—OH)—C₃-C₇ cycloalkyl, or 5- to 6-membered heteroaryl;
each R⁵ is independently F, Cl, —SCF₃, —SCHF₂, —SF₅, —OCF₃, —OR⁶, —N(R⁶)R⁶, —N₃, —OCHF₂, —CF₃, —CF₂Cl, —CHF₂, —CN, —NO₂, C₁-C₆ alkyl, C₁-C₆ haloalkyl, C₁-C₆ heteroalkyl, or C₃-C₇ cycloalkyl,
   wherein the aliphatic portions of R⁵ are optionally substituted with 1 to 3 R³ groups; and
each R⁶ is independently H, C₁-C₆ alkyl, C₁-C₆ haloalkyl, C₁-C₆ heteroalkyl, C₃-C₇ cycloalkyl, C₆ aryl, 5- to 10-membered heteroaryl, or 4- to 7-membered heterocycloalkyl.

In one aspect, provided is a compound of formula (I-2)

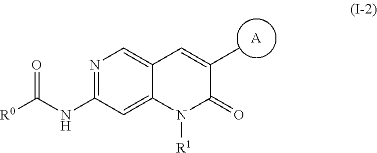

or a pharmaceutically acceptable salt, solvate, hydrate, or co-crystal thereof, or a mixture of any of the foregoing, wherein:
R⁰ is C₁-C₃ alkyl, wherein the C₁-C₃ alkyl is optionally substituted with 1 to 5 R⁵ groups;
R¹ is H, C₁-C₆ alkyl, C₁-C₆ haloalkyl, C₁-C₆ heteroalkyl, C₃-C₇ cycloalkyl, C₁-C₆ alkyl-CN, 4- to 7-membered heterocycloalkyl, —(X^{a})₀₋₁—(C(R²)R²)₀₋₁—OR²,
   —(X^{a})₀₋₁—(C(R²)R²)₀₋₁—N(R²)R², —(X^{a})₀₋₁—(C(R²)R²)₀₋₁—S(O)₂R²,
   —(X^{a})—(C(R²)R²)₀₋₁—S(O)(═NR²)R², —(X^{a})—C(R²)R²—S(O)₂N(R²)R²,
   —(X^{a})—C(R²)R²—S(O)R², —(X^{a})—C(R²)R²—N(R²)R²S(O)₂R², or
   —(X^{a})—(C(R²)R²)₀₋₁—C(O)N(R²)R²,
   wherein the aliphatic portions of R¹ are optionally substituted with 1 to 5 R³ groups;
X^{a} is C₁-C₆ alkylene, C₁-C₆ heteroalkylene, C₃-C₇ cycloalkylene, or C₄-C₇ heterocycloalkylene, each of which is optionally substituted with 1 to 5 R³ groups;
each R² is independently H, C₁-C₆ alkyl, C₁-C₆ haloalkyl, C₁-C₆ heteroalkyl, C₃-C₇ cycloalkyl, 4- to 7-membered heterocycloalkyl, or 5- to 6-membered heteroaryl,
   wherein the aliphatic and aromatic portions of R² are optionally substituted with 1 to 5 R³ groups, and
   wherein any two R² groups attached to the same nitrogen are optionally taken together to form a 4- to 7-membered heterocyclic ring, or any two R² groups attached to the same carbon atom are optionally taken together to form a 3- to 6-membered carbocyclic ring or a 4- to 6-membered heterocyclic ring optionally containing 1-3 heteroatoms selected from the group consisting of N, O, and S, wherein each carbocyclic or heterocyclic ring is optionally substituted with 1 to 5 R³ groups;
each R³ is independently C₁-C₆ alkyl, C₁-C₆-alkoxy, C₃-C₇ cycloalkyl, C₃-C₇ cycloalkoxy,
   C₁-C₆ haloalkyl, C₁-C₆ alkylamino, C₁-C₆ dialkylamino, 4- to 7-membered heterocycloalkyl, 4- to 7-membered heterocycloalkoxy, 5- to 6-membered heteroaryl, F, Cl, —OH, —NH₂, —NHMe, —NMe₂, —SH, —SMe, —S(O)Me, —S(O)₂Me,
   —S(O)(═NH)Me, —S(O)(═NMe)Me, —CN, —NO₂, —CHF₂, —CF₃, —CF₂Cl, —OCF₃, —OCHF₂, —SCF₃, —SCHF₂, —SF₅, —P(O)(Me)₂, or —N₃;
Ring A is C₆-C₁₀ aryl or 5- to 10-membered heteroaryl,
   wherein the aromatic portions of Ring A are optionally substituted with 1 to 5 R⁴ groups, and
   wherein when Ring A is C₆ aryl or 5- to 6-membered heteroaryl, then any two substituents attached to adjacent atoms of said aryl or heteroaryl are optionally taken together to form a 4- to 6-membered carbocyclic ring or a 4- to 6-membered heterocyclic ring optionally containing 1-3 heteroatoms selected from the group consisting of N, O, and S, wherein each carbocyclic or heterocyclic ring is optionally substituted with 1 to 5 R⁴ groups;
each R⁴ is independently F, Cl, Br, I, —SCF₃, —SCHF₂, —SF₅, —OCF₃, —OR⁶, —N(R⁶)R⁶, —N₃, —OCHF₂, —CF₃, —CF₂Cl, —CHF₂, —CN, —NO₂, —C(═O)C₁-C₆ alkyl, C₁-C₆ alkyl, C₁-C₆ haloalkyl, C₁-C₆ haloalkyl-OH, C₁-C₆ alkyl-OH, C₁-C₆ alkyl-OR⁶, C₁-C₆ alkylene-C₁-C₃ alkoxy, C₁-C₆ alkylene(—OH)—C₁-C₃ alkoxy, C₁-C₆ alkyl-CN, C₁-C₆ heteroalkyl, C₃-C₇ cycloalkyl, C₁-C₆ alkylene-C₃-C₇ cycloalkyl, C₁-C₆ alkylene(—OH)—C₃-C₇ cycloalkyl, or 5- to 6-membered heteroaryl;
each R⁵ is independently F, Cl, —SCF₃, —SCHF₂, —SF₅, —OCF₃, —OR⁶, —N(R⁶)R⁶, —N₃, —OCHF₂, —CF₃, —CF₂Cl, —CHF₂, —CN, —NO₂, C₁-C₆ alkyl, C₁-C₆ haloalkyl, C₁-C₆ heteroalkyl, or C₃-C₇ cycloalkyl,
   wherein the aliphatic portions of R⁵ are optionally substituted with 1 to 3 R³ groups; and
each R⁶ is independently H, C₁-C₆ alkyl, C₁-C₆ haloalkyl, C₁-C₆ heteroalkyl, C₃-C₇ cycloalkyl, C₆ aryl, 5- to 10-membered heteroaryl, or 4- to 7-membered heterocycloalkyl.

In one aspect, provided herein is a compound of formula (I-1)

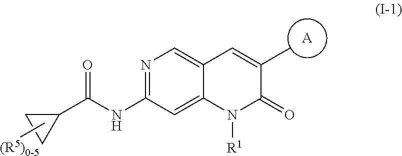

or a pharmaceutically acceptable salt, solvate, hydrate, or co-crystal thereof, or a mixture of any of the foregoing, wherein:
R¹ is H, C₁-C₆ alkyl, C₁-C₆ haloalkyl, C₁-C₆ heteroalkyl, C₃-C₇ cycloalkyl, C₁-C₆ alkyl-CN, 4- to 7-membered heterocycloalkyl, —(X^{a})₀₋₁—(C(R²)R²)₀₋₁—OR²,
   —(X^{a})₀₋₁—(C(R²)R²)₀₋₁—N(R²)R², —(X^{a})₀₋₁—(C(R²)R²)₀₋₁—S(O)₂R²,
   —(X^{a})—(C(R²)R²)₀₋₁—S(O)(═NR²)R², —(X^{a})—C(R²)R²—S(O)₂N(R²)R²,
   —(X^{a})—C(R²)R²—S(O)R², —(X^{a})—C(R²)R²—N(R²)R²S(O)₂R², or
   —(X^{a})—(C(R²)R²)₀₋₁—C(O)N(R²)R²,
   wherein the aliphatic portions of R¹ are optionally substituted with 1 to 5 R³ groups;
X^{a} is C₁-C₆ alkylene, C₁-C₆ heteroalkylene, C₃-C₇ cycloalkylene, or C₄-C₇ heterocycloalkylene, each of which is optionally substituted with 1 to 5 R³ groups;
each R² is independently H, C₁-C₆ alkyl, C₁-C₆ haloalkyl, C₁-C₆ heteroalkyl, C₃-C₇ cycloalkyl, 4- to 7-membered heterocycloalkyl, or 5- to 6-membered heteroaryl,
   wherein the aliphatic and aromatic portions of R² are optionally substituted with 1 to 5 R³ groups, and
   wherein any two R² groups attached to the same nitrogen are optionally taken together to form a 4- to 7-membered heterocyclic ring, or any two R² groups attached to the same carbon atom are optionally taken together to form a 3- to 6-membered carbocyclic ring or a 4- to 6-membered heterocyclic ring optionally containing 1-3 heteroatoms selected from the group consisting of N, O, and S, wherein each carbocyclic or heterocyclic ring is optionally substituted with 1 to 5 R³ groups;
each R³ is independently C₁-C₆ alkyl, C₁-C₆-alkoxy, C₃-C₇ cycloalkyl, C₃-C₇ cycloalkoxy,
   C₁-C₆ haloalkyl, C₁-C₆ alkylamino, C₁-C₆ dialkylamino, 4- to 7-membered heterocycloalkyl, 4- to 7-membered heterocycloalkoxy, 5- to 6-membered heteroaryl, F, Cl, —OH, —NH₂, —NHMe, —NMe₂, —SH, —SMe, —S(O)Me, —S(O)₂Me,
   —S(O)(═NH)Me, —S(O)(═NMe)Me, —CN, —NO₂, —CHF₂, —CF₃, —CF₂Cl, —OCF₃, —OCHF₂, —SCF₃, —SCHF₂, —SF₅, —P(O)(Me)₂, or —N₃;
Ring A is C₆-C₁₀ aryl or 5- to 10-membered heteroaryl,
   wherein the aromatic portions of Ring A are optionally substituted with 1 to 5 R⁴ groups, and
   wherein when Ring A is C₆ aryl or 5- to 6-membered heteroaryl, then any two substituents attached to adjacent atoms of said aryl or heteroaryl are optionally taken together to form a 4- to 6-membered carbocyclic ring or a 4- to 6-membered heterocyclic ring optionally containing 1-3 heteroatoms selected from the group consisting of N, O, and S, wherein each carbocyclic or heterocyclic ring is optionally substituted with 1 to 5 R⁴ groups;
each R⁴ is independently H, F, Cl, Br, I, —SCF₃, —SCHF₂, —SF₅, —OCF₃, —OR⁶, —N(R⁶)R⁶, —N₃, —OCHF₂, —CF₃, —CF₂Cl, —CHF₂, —CN, —NO₂, C₁-C₆ alkyl, C₁-C₆ haloalkyl, C₁-C₆ haloalkyl-OH, C₁-C₆ alkyl-OH, C₁-C₆ alkyl-CN, C₁-C₆ heteroalkyl, or C₃-C₇ cycloalkyl;
each R⁵ is independently F, Cl, —SCF₃, —SCHF₂, —SF₅, —OCF₃, —OR⁶, —N(R⁶)R⁶, —N₃, —OCHF₂, —CF₃, —CF₂Cl, —CHF₂, —CN, —NO₂, C₁-C₆ alkyl, C₁-C₆ haloalkyl, C₁-C₆ heteroalkyl, or C₃-C₇ cycloalkyl,
   wherein the aliphatic portions of R⁵ are optionally substituted with 1 to 3 R³ groups; and
each R⁶ is independently H, C₁-C₆ alkyl, C₁-C₆ haloalkyl, C₁-C₆ heteroalkyl, C₃-C₇ cycloalkyl, C₆ aryl, 5- to 10-membered heteroaryl, or 4- to 7-membered heterocycloalkyl.

In one aspect, provided herein is a compound of formula (I-2)

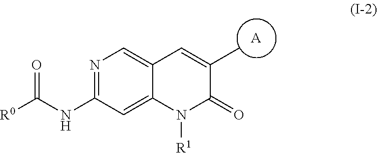

or a pharmaceutically acceptable salt, solvate, hydrate, or co-crystal thereof, or a mixture of any of the foregoing, wherein:
R⁰ is C₁-C₃ alkyl, wherein the C₁-C₃ alkyl is optionally substituted with 1 to 5 R⁵ groups;
R¹ is H, C₁-C₆ alkyl, C₁-C₆ haloalkyl, C₁-C₆ heteroalkyl, C₃-C₇ cycloalkyl, C₁-C₆ alkyl-CN, 4- to 7-membered heterocycloalkyl, —(X^{a})₀₋₁—(C(R²)R²)₀₋₁—OR²,
   —(X^{a})₀₋₁—(C(R²)R²)₀₋₁—N(R²)R², —(X^{a})₀₋₁—(C(R²)R²)₀₋₁—S(O)₂R²,
   —(X^{a})—(C(R²)R²)₀₋₁—S(O)(═NR²)R², —(X^{a})—C(R²)R²—S(O)₂N(R²)R²,
   —(X^{a})—C(R²)R²—S(O)R², —(X^{a})—C(R²)R²—N(R²)R²S(O)₂R², or
   —(X^{a})—(C(R²)R²)₀₋₁—C(O)N(R²)R²,
   wherein the aliphatic portions of R¹ are optionally substituted with 1 to 5 R³ groups;
X^{a} is C₁-C₆ alkylene, C₁-C₆ heteroalkylene, C₃-C₇ cycloalkylene, or C₄-C₇ heterocycloalkylene, each of which is optionally substituted with 1 to 5 R³ groups;
each R² is independently H, C₁-C₆ alkyl, C₁-C₆ haloalkyl, C₁-C₆ heteroalkyl, C₃-C₇ cycloalkyl, 4- to 7-membered heterocycloalkyl, or 5- to 6-membered heteroaryl,
   wherein the aliphatic and aromatic portions of R² are optionally substituted with 1 to 5 R³ groups, and
   wherein any two R² groups attached to the same nitrogen are optionally taken together to form a 4- to 7-membered heterocyclic ring, or any two R² groups attached to the same carbon atom are optionally taken together to form a 3- to 6-membered carbocyclic ring or a 4- to 6-membered heterocyclic ring optionally containing 1-3 heteroatoms selected from the group consisting of N, O, and S, wherein each carbocyclic or heterocyclic ring is optionally substituted with 1 to 5 R³ groups;
each R³ is independently C₁-C₆ alkyl, C₁-C₆-alkoxy, C₃-C₇ cycloalkyl, C₃-C₇ cycloalkoxy,
   C₁-C₆ haloalkyl, C₁-C₆ alkylamino, C₁-C₆ dialkylamino, 4- to 7-membered heterocycloalkyl, 4- to 7-membered heterocycloalkoxy, 5- to 6-membered heteroaryl, F, Cl, —OH, —NH₂, —NHMe, —NMe₂, —SH, —SMe, —S(O)Me, —S(O)₂Me,
   —S(O)(═NH)Me, —S(O)(═NMe)Me, —CN, —NO₂, —CHF₂, —CF₃, —CF₂Cl, —OCF₃, —OCHF₂, —SCF₃, —SCHF₂, —SF₅, —P(O)(Me)₂, or —N₃;
Ring A is C₆-C₁₀ aryl or 5- to 10-membered heteroaryl,
   wherein the aromatic portions of Ring A are optionally substituted with 1 to 5 R⁴ groups, and
   wherein when Ring A is C₆ aryl or 5- to 6-membered heteroaryl, then any two substituents attached to adjacent atoms of said aryl or heteroaryl are optionally taken together to form a 4- to 6-membered carbocyclic ring or a 4- to 6-membered heterocyclic ring optionally containing 1-3 heteroatoms selected from the group consisting of N, O, and S, wherein each carbocyclic or heterocyclic ring is optionally substituted with 1 to 5 R⁴ groups;
each R⁴ is independently H, F, Cl, Br, I, —SCF₃, —SCHF₂, —SF₅, —OCF₃, —OR⁶, —N(R⁶)R⁶, —N₃, —OCHF₂, —CF₃, —CF₂Cl, —CHF₂, —CN, —NO₂, C₁-C₆ alkyl, C₁-C₆ haloalkyl, C₁-C₆ haloalkyl-OH, C₁-C₆ alkyl-OH, C₁-C₆ alkyl-CN, C₁-C₆ heteroalkyl, or C₃-C₇ cycloalkyl;
each R⁵ is independently F, Cl, —SCF₃, —SCHF₂, —SF₅, —OCF₃, —OR⁶, —N(R⁶)R⁶, —N₃, —OCHF₂, —CF₃, —CF₂Cl, —CHF₂, —CN, —NO₂, C₁-C₆ alkyl, C₁-C₆ haloalkyl, C₁-C₆ heteroalkyl, or C₃-C₇ cycloalkyl,
   wherein the aliphatic portions of R⁵ are optionally substituted with 1 to 3 R³ groups; and
each R⁶ is independently H, C₁-C₆ alkyl, C₁-C₆ haloalkyl, C₁-C₆ heteroalkyl, C₃-C₇ cycloalkyl, C₆ aryl, 5- to 10-membered heteroaryl, or 4- to 7-membered heterocycloalkyl.

In some embodiments, R¹ is H, C₁-C₆ alkyl, C₁-C₆ haloalkyl, C₁-C₆ heteroalkyl,
C₃-C₇ cycloalkyl, C₁-C₆ alkyl-CN, 4- to 7-membered heterocycloalkyl, —(X^{a})₀₋₁—(C(R²)R²)₀₋₁—OR², or
—(X^{a})₀₋₁—(C(R²)R²)₀₋₁—N(R²)R², wherein the aliphatic portions of R¹ are optionally substituted with 1 to 5 R³ groups. In some embodiments, R¹ is H, C₁-C₆ alkyl, C₁-C₆ haloalkyl, C₁-C₆ alkyl-CN, or C₁-C₆ heteroalkyl, wherein the aliphatic portions of R¹ are optionally substituted with 1 to 5 R³ groups. In some embodiments, R¹ is H or C₁-C₆ alkyl optionally substituted with 1 to 5 R³ groups. In some embodiments, R¹ is —CH₃.

In some embodiments, R¹ is H.

In some embodiments, R¹ is C₁-C₆ alkyl optionally substituted with 1 to 5 R³ groups. In some embodiments, R¹ is unsubstituted C₁-C₆ alkyl. In some embodiments, R¹ is C₁-C₆ alkyl substituted with 1 to 5 R³ groups. In some embodiments, R¹ is C₁-C₃ alkyl optionally substituted with 1 to 3 R³ groups. In some embodiments, R¹ is methyl, ethyl, n-propyl, or isopropyl. In some embodiments, R¹ is —CH₃ or —CH₂CH₃. In some embodiments, R¹ is —CH₃. In some embodiments, R¹ is —CH₂CH₃.

In some embodiments, R¹ is C₁-C₆ haloalkyl optionally substituted with 1 to 5 R³ groups. In some embodiments, R¹ is C₁-C₆ haloalkyl which is not substituted by any R³ groups. In some embodiments, R¹ is C₁-C₆ haloalkyl substituted with 1 to 5 R³ groups. In some embodiments, R¹ is C₁-C₆ haloalkyl containing 1-13 halogen atoms. In some embodiments, R¹ is C₁-C₃ haloalkyl. In some embodiments, R¹ is C₁-C₃ haloalkyl containing 1-7 halogen atoms. In some embodiments, R¹ is C₁-C₂ haloalkyl containing 1-5 halogen atoms. In some embodiments, the halogen atoms are independently selected from the group consisting of chloro, bromo, and fluoro atoms. In some embodiments, the halogen atoms are independently selected from the group consisting of chloro and fluoro atoms. In some embodiments, the halogen atoms are all fluoro atoms. In some embodiments, the halogen atoms are all chloro atoms. In some embodiments, the halogen atoms are a combination of chloro and fluoro atoms. In some embodiments, R¹ is —CF₃, —CCl₃, —CF₂Cl, —CFCl₂, —CHF₂, —CH₂F, —CHC₁₂, —CH₂F, or —CHFCl. In some embodiments, R¹ is —CF₃.

In some embodiments, R¹ is C₁-C₆ alkyl-CN. In some embodiments, R¹ is C₁-C₆ alkyl-CN containing 1-6 cyano groups. In some embodiments, R¹ is C₁-C₆ alkyl-CN containing 1 cyano group. In some embodiments, R¹ is C₁-C₆ alkyl-CN containing 2 cyano groups. In some embodiments, R¹ is C₁-C₆ alkyl-CN containing 3 cyano groups. In some embodiments, R¹ is C₁-C₃ alkyl-CN. In some embodiments, R¹ is C₁-C₃ alkyl-CN containing 1-3 cyano groups. In some embodiments, R¹ is C₁-C₃ alkyl-CN containing 1 cyano group. In some embodiments, R¹ is —CH₂CN, —CH₂CH₂CN, —CH(CN)CH₃, —CH₂CH₂CH₂CN, —CH(CN)CH₂CH₃, or
—CH₂CH(CN)CH₃. In some embodiments, R¹ is —CH₂CN or —CH₂CH₂CN. In some embodiments, R¹ is —CH₂CN. In some embodiments, R¹ is —CH₂CH₂CN.

In some embodiments, R¹ is C₁-C₆ heteroalkyl optionally substituted with 1 to 5 R³ groups. In some embodiments, R¹ is C₁-C₆ heteroalkyl which is not substituted by any R³ groups. In some embodiments, R¹ is C₁-C₆ heteroalkyl substituted with 1 to 5 R³ groups. In some embodiments, R¹ is C₁-C₆ heteroalkyl containing 1-3 heteroatoms selected from the group consisting of N and O. In some embodiments, R¹ is C₁-C₆ heteroalkyl containing 1 nitrogen atom. In some embodiments, R¹ is C₁-C₆ heteroalkyl containing 1 oxygen atom. In some embodiments, R¹ is C₁-C₃ heteroalkyl optionally substituted with 1 to 3 R³ groups. In some embodiments, R¹ is —CH₂—CH₂—O—CH₃, —CH₂—O—CH₃, —CH₂—CH₂—NH—CH₃, or —CH₂—NH—CH₃.

In some embodiments, R¹ is C₃-C₇ cycloalkyl optionally substituted with 1 to 5 R³ groups. In some embodiments, R¹ is C₃-C₇ cycloalkyl which is not substituted by any R³ groups. In some embodiments, R¹ is C₃-C₆ cycloalkyl substituted with 1 to 5 R³ groups. In some embodiments, R¹ is cyclopropyl, cyclobutyl, cyclopentyl, or cyclohexyl, each of which is optionally substituted with 1 to 5 R³ groups. In some embodiments, R¹ is cyclopropyl. In some embodiments, R¹ is —CH₂-cyclopropyl.

In some embodiments, R¹ is 4- to 7-membered heterocycloalkyl optionally substituted with 1 to 5 R³ groups. In some embodiments, R¹ is 4- to 7-membered heterocycloalkyl which is not substituted by any R³ groups. In some embodiments, R¹ is 4- to 7-membered heterocycloalkyl substituted with 1 to 5 R³ groups. In some embodiments, R¹ is 4- to 6-membered heterocycloalkyl optionally substituted with 1 to 5 R³ groups. In some embodiments, R¹ is 4- to 7-membered heterocycloalkyl containing 1-3 heteroatoms selected from the group consisting of N and O. In some embodiments, R¹ is 4- to 7-membered heterocycloalkyl containing 1 nitrogen atom. In some embodiments, R¹ is 4- to 7-membered heterocycloalkyl containing 1 oxygen atom. In some embodiments, R¹ is pyrrolidinyl or piperidinyl.

In some embodiments, R¹ is —(X^{a})₀₋₁—(C(R²)R²)₀₋₁—OR², —(X^{a})₀₋₁—(C(R²)R²)₀₋₁—N(R²)R²,
—(X^{a})₀₋₁—(C(R²)R²)₀₋₁—OR², —(X^{a})₀₋₁—(C(R²)R²)₀₋₁—N(R²)R², —(X^{a})₀₋₁—(C(R²)R²)_{0- 1}—S(O)₂R², —(X^{a})—(C(R²)R²)₀₋₁—S(O)(═NR²)R², —(X^{a})—C(R²)R²—S(O)₂N(R²)R², —(X^{a})—C(R²)R²—S(O)R², —(X^{a})—C(R²)R²—N(R²)R²S(O)₂R², or —(X^{a})—(C(R²)R²)₀₋₁—C(O)N(R²)R², wherein the aliphatic portions of R¹ are optionally substituted with 1 to 5 R³ groups. In some embodiments, R¹ is —(X^{a})₀₋₁—(C(R²)R²)₀₋₁—OR², —(X^{a})₀₋₁—(C(R²)R²)₀₋₁—N(R²)R², —(X^{a})₀₋₁—(C(R²)R²)₀₋₁—OR², —(X^{a})₀₋₁—(C(R²)R²)₀₋₁—N(R²)R², —(X^{a})₀₋₁—(C(R²)R²)₀₋₁—S(O)₂R², —(X^{a})—(C(R²)R²)₀₋₁—S(O)(═NR²)R²,
—(X^{a})—C(R²)R²—S(O)₂N(R²)R², —(X^{a})—C(R²)R²—S(O)R², —(X^{a})—C(R²)R²—N(R²)R²S(O)₂R², or —(X^{a})—(C(R²)R²)₀₋₁—C(O)N(R²)R², wherein the aliphatic portions of R¹ are optionally substituted with 1 to 5 R³ groups. In some embodiments, R¹ is —(X^{a})₀₋₁—(C(R²)R²)₀₋₁—OR² or —(X^{a})₀₋₁—(C(R²)R²)₀₋₁—N(R²)R², wherein the aliphatic portions of R¹ are optionally substituted with 1 to 5 R³ groups.

In some embodiments, X^{a} is C₁-C₆ alkylene optionally substituted with 1 to 5 R³ groups. In some embodiments, X^{a} is C₁-C₆ alkylene which is not substituted with any R³ groups. In some embodiments, X^{a} is C₁-C₆ alkylene substituted with 1 to 5 R³ groups. In some embodiments, X^{a} is C₁-C₃ alkylene optionally substituted with 1 to 3 R³ groups. In some embodiments, X^{a} is —CH₂—, —CH₂CH₂—, or —CH₂CH₂CH₂—.

In some embodiments, X^{a} is C₁-C₆ heteroalkylene optionally substituted with 1 to 5 R³ groups. In some embodiments, X^{a} is C₁-C₆ heteroalkylene which is not substituted with any R³ groups. In some embodiments, X^{a} is C₁-C₆ heteroalkylene substituted with 1 to 5 R³ groups. In some embodiments, X^{a} is C₁-C₆ heteroalkylene containing 1-3 heteroatoms selected from the group consisting of N and O. In some embodiments, X^{a} is C₁-C₆ heteroalkylene containing 1 nitrogen atom. In some embodiments, X^{a} is C₁-C₆ heteroalkylene containing 1 oxygen atom. In some embodiments, X^{a} is C₁-C₃ heteroalkylene optionally substituted with 1 to 3 R³ groups. In some embodiments, X^{a} is —CH₂—NH—CH₂—, —CH₂CH₂—NH—CH₂—, or —CH₂—NH—CH₂CH₂—.

In some embodiments, X^{a} is C₃-C₇ cycloalkylene optionally substituted with 1 to 5 R³ groups. In some embodiments, X^{a} is C₃-C₇ cycloalkylene which is not substituted with any R³ groups. In some embodiments, X^{a} is C₃-C₇ cycloalkylene substituted with 1 to 5 R³ groups. In some embodiments, X^{a} is C₃-C₆ cycloalkylene optionally substituted with 1 to 3 R³ groups. In some embodiments, X^{a} is cyclopropylene, cyclobutylene, cyclopentylene, or cyclohexylene.

In some embodiments, X^{a} is C₃-C₇ heterocycloalkylene optionally substituted with 1 to 5 R³ groups. In some embodiments, X^{a} is C₃-C₇ heterocycloalkylene which is not substituted with any R³ groups. In some embodiments, X^{a} is C₃-C₇ heterocycloalkylene substituted with 1 to 5 R³ groups. In some embodiments, X^{a} is C₃-C₆ heterocycloalkylene optionally substituted with 1 to 3 R³ groups. In some embodiments, X^{a} is pyrrolidinylene or piperidinylene.

In some embodiments, each R² is independently H, C₁-C₆ alkyl, C₁-C₆ haloalkyl, C₁-C₆ heteroalkyl, C₃-C₇ cycloalkyl, 4- to 7-membered heterocycloalkyl, or 5- to 6-membered heteroaryl, wherein the aliphatic and aromatic portions of R² are optionally substituted with 1 to 5 R³ groups.

In some embodiments, R² is H.

In some embodiments, R² is C₁-C₆ alkyl optionally substituted with 1 to 5 R³ groups. In some embodiments, R² is unsubstituted C₁-C₆ alkyl. In some embodiments, R² is C₁-C₆ alkyl substituted with 1 to 5 R³ groups. In some embodiments, R² is C₁-C₃ alkyl optionally substituted with 1 to 3 R³ groups. In some embodiments, R² is methyl, ethyl, n-propyl, or isopropyl. In some embodiments, R² is —CH₃.

In some embodiments, R² is C₁-C₆ haloalkyl optionally substituted with 1 to 5 R³ groups. In some embodiments, R² is C₁-C₆ haloalkyl which is not substituted by any R³ groups. In some embodiments, R² is C₁-C₆ haloalkyl substituted with 1 to 5 R³ groups. In some embodiments, R² is C₁-C₆ haloalkyl containing 1-13 halogen atoms. In some embodiments, R² is C₁-C₃ haloalkyl. In some embodiments, R² is C₁-C₃ haloalkyl containing 1-7 halogen atoms. In some embodiments, R² is C₁-C₂ haloalkyl containing 1-5 halogen atoms. In some embodiments, the halogen atoms are independently selected from the group consisting of chloro, bromo, and fluoro atoms. In some embodiments, the halogen atoms are independently selected from the group consisting of chloro and fluoro atoms. In some embodiments, the halogen atoms are all fluoro atoms. In some embodiments, the halogen atoms are all chloro atoms. In some embodiments, the halogen atoms are a combination of chloro and fluoro atoms. In some embodiments, R² is —CF₃, —CCl₃, —CF₂Cl, —CFCl₂, —CHF₂, —CH₂F, —CHC₁₂, —CH₂F, or —CHFCl. In some embodiments, R² is —CF₃.

In some embodiments, R² is C₁-C₆ heteroalkyl optionally substituted with 1 to 5 R³ groups. In some embodiments, R² is C₁-C₆ heteroalkyl which is not substituted by any R³ groups. In some embodiments, R² is C₁-C₆ heteroalkyl substituted with 1 to 5 R³ groups. In some embodiments, R² is C₁-C₆ heteroalkyl containing 1-3 heteroatoms selected from the group consisting of N and O. In some embodiments, R² is C₁-C₆ heteroalkyl containing 1 nitrogen atom. In some embodiments, R² is C₁-C₆ heteroalkyl containing 1 oxygen atom. In some embodiments, R² is C₁-C₃ heteroalkyl optionally substituted with 1 to 3 R³ groups. In some embodiments, R² is —CH₂—CH₂—O—CH₃, —CH₂—O—CH₃, —CH₂—CH₂—NH—CH₃, or —CH₂—NH—CH₃.

In some embodiments, R² is C₃-C₇ cycloalkyl optionally substituted with 1 to 5 R³ groups. In some embodiments, R² is C₃-C₇ cycloalkyl which is not substituted by any R³ groups. In some embodiments, R² is C₃-C₆ cycloalkyl substituted with 1 to 5 R³ groups. In some embodiments, R² is cyclopropyl, cyclobutyl, cyclopentyl, or cyclohexyl, each of which is optionally substituted with 1 to 5 R³ groups. In some embodiments, R² is cyclopropyl.

In some embodiments, R² is 4- to 7-membered heterocycloalkyl optionally substituted with 1 to 5 R³ groups. In some embodiments, R² is 4- to 7-membered heterocycloalkyl which is not substituted by any R³ groups. In some embodiments, R² is 4- to 7-membered heterocycloalkyl substituted with 1 to 5 R³ groups. In some embodiments, R² is 4- to 6-membered heterocycloalkyl optionally substituted with 1 to 5 R³ groups. In some embodiments, R² is 4- to 7-membered heterocycloalkyl containing 1-3 heteroatoms selected from the group consisting of N and O. In some embodiments, R² is 4- to 7-membered heterocycloalkyl containing 1 nitrogen atom. In some embodiments, R² is 4- to 7-membered heterocycloalkyl containing 1 oxygen atom. In some embodiments, R² is pyrrolidinyl or piperidinyl.

In some embodiments, R² is 5- to 6-membered heteroaryl optionally substituted with 1 to 5 R³ groups. In some embodiments, R² is unsubstituted 5- to 6-membered heteroaryl. In some embodiments, R² is 5- to 6-membered heteroaryl substituted with 1 to 5 R³ groups. In some embodiments, R² is 5- to 6-membered heteroaryl containing 1-3 heteroatoms selected from the group consisting of O, N, and S. In some embodiments, R² is a 5-membered heteroaryl optionally substituted with 1 to 5 R³ groups. In some embodiments, R² is a 6-membered heteroaryl optionally substituted with 1 to 5 R³ groups. In some embodiments, R² is pyridyl, pyridazinyl, pyrimindinyl, or pyrazolyl.

In some embodiments, two R² groups attached to the same nitrogen are taken together to form a 4- to 7-membered heterocyclic ring optionally substituted with 1 to 5 R³ groups. In some embodiments, two R² groups attached to the same nitrogen are taken together to form a 5- to 6-membered heterocyclic ring optionally substituted with 1 to 5 R³ groups. In some embodiments, two R² groups attached to the same carbon atom are taken together to form a 3- to 6-membered carbocyclic ring optionally substituted with 1 to 5 R³ groups. In some embodiments, two R² groups attached to the same carbon atom are taken together to form a 3- to 5-membered carbocyclic ring optionally substituted with 1 to 5 R³ groups. In some embodiments, two R² groups attached to the same carbon atom are taken together to form a 4- to 6-membered heterocyclic ring containing 1-3 heteroatoms selected from the group consisting of N, O, and S, and optionally substituted with 1 to 5 R³ groups. In some embodiments, two R² groups attached to the same carbon atom are taken together to form a 5- to 6-membered heterocyclic ring containing 1-3 heteroatoms selected from the group consisting of N, O, and S, and optionally substituted with 1 to 5 R³ groups.

In some embodiments, each R³ is independently C₁-C₆ alkyl, C₁-C₆-alkoxy, C₃-C₇ cycloalkyl, C₃-C₇ cycloalkoxy, C₁-C₆ haloalkyl, C₁-C₆ alkylamino, C₁-C₆ dialkylamino, 4- to 7-membered heterocycloalkyl, 4- to 7-membered heterocycloalkoxy, 5- to 6-membered heteroaryl, F, Cl, —OH, —NH₂, —NHMe, —NMe₂, —SMe, —S(O)Me, —S(O)₂Me, or —CN.

In some embodiments, R³ is C₁-C₆ alkyl. In some embodiments, R³ is C₁-C₃ alkyl. In some embodiments, R³ is methyl, ethyl, n-propyl, or isopropyl. In some embodiments, R³ is —CH₃.

In some embodiments, R³ is C₁-C₆ alkoxy. In some embodiments, R³ is C₁-C₃ alkoxy. In some embodiments, R³ is —OCH₃, —CH₂CH₃, —OCH₂CH₂CH₃, or —OCH(CH₃)₂. In some embodiments, R³ is —OCH₃.

In some embodiments, R³ is C₃-C₇ cycloalkyl. In some embodiments, R³ is C₃-C₆ cycloalkyl. In some embodiments, R³ is cyclopropyl, cyclobutyl, cyclopentyl, or cyclohexyl.

In some embodiments, R³ is C₃-C₇ cycloalkoxy. In some embodiments, R³ is C₃-C₆ cycloalkoxy. In some embodiments, R³ is —O(cyclopropyl), —O(cyclobutyl), —O(cyclopentyl), or —O(cyclohexyl).

In some embodiments, R³ is C₁-C₆ haloalkyl. In some embodiments, R³ is C₁-C₆ haloalkyl containing 1-13 halogen atoms. In some embodiments, R³ is C₁-C₃ haloalkyl. In some embodiments, R³ is C₁-C₃ haloalkyl containing 1-7 halogen atoms. In some embodiments, R³ is C₁-C₂ haloalkyl containing 1-5 halogen atoms. In some embodiments, the halogen atoms are independently selected from the group consisting of chloro, bromo, and fluoro atoms. In some embodiments, the halogen atoms are independently selected from the group consisting of chloro and fluoro atoms. In some embodiments, the halogen atoms are all fluoro atoms. In some embodiments, the halogen atoms are all chloro atoms. In some embodiments, the halogen atoms are a combination of chloro and fluoro atoms. In some embodiments, R³ is —CF₃, —CCl₃, —CF₂Cl, —CFCl₂, —CHF₂, —CH₂F, —CHCl₂, —CH₂F, or —CHFCl. In some embodiments, R³ is —CF₃.

In some embodiments, R³ is C₁-C₆ alkylamino. In some embodiments, R³ is C₁-C₃ alkylamino. In some embodiments, R³ is C₁-C₂ alkylamino. In some embodiments, R³ is —NH(CH₃) or —NH(CH₂CH₃).

In some embodiments, R³ is C₁-C₆ dialkylamino. In some embodiments, R³ is C₁-C₆ dialkylamino wherein the two C₁-C₆ alkyl groups are the same. In some embodiments, R³ is C₁-C₆ dialkylamino wherein the two C₁-C₆ alkyl groups are different. In some embodiments, R³ is C₁-C₃ dialkylamino. In some embodiments, R³ is —N(CH₃)₂, —N(CH₂CH₃)₂, or —N(CH₃)(CH₂CH₃).

In some embodiments, R³ is 4- to 7-membered heterocycloalkyl. In some embodiments, R³ is 4- to 7-membered heterocycloalkyl containing 1-3 heteroatoms selected from the group consisting of N and O. In some embodiments, R³ is 4- to 7-membered heterocycloalkyl containing 1-2 nitrogen atoms. In some embodiments, R³ is 4- to 7-membered heterocycloalkyl containing 1-2 oxygen atoms. In some embodiments, R³ is 4- to 7-membered heterocycloalkyl containing 1 oxygen atom and 1 nitrogen atom. In some embodiments, R³ is 4- to 6-membered heterocycloalkyl. In some embodiments, R³ is pyrrolidinyl or piperidinyl.

In some embodiments, R³ is 4- to 7-membered heterocycloalkoxy. In some embodiments, R³ is 4- to 7-membered heterocycloalkoxy containing 1-3 heteroatoms selected from the group consisting of N and O. In some embodiments, R³ is 4- to 7-membered heterocycloalkoxy containing 1-2 nitrogen atoms. In some embodiments, R³ is 4- to 7-membered heterocycloalkoxy containing 1-2 oxygen atoms. In some embodiments, R³ is 4- to 7-membered heterocycloalkoxy containing 1 oxygen atom and 1 nitrogen atom. In some embodiments, R³ is 4- to 6-membered heterocycloalkoxy. In some embodiments, R³ is —O(pyrrolidinyl) or —O(piperidinyl).

In some embodiments, R³ is 5- to 6-membered heteroaryl. In some embodiments, R³ is 5- to 6-membered heteroaryl containing 1-3 heteroatoms selected from the group consisting of 0, N, and S. In some embodiments, R³ is 5- to 6-membered heteroaryl containing 1-2 heteroatoms selected from the group consisting of 0 and N. In some embodiments, R³ is 5- to 6-membered heteroaryl containing 1-2 nitrogen atoms. In some embodiments, R³ is 5- to 6-membered heteroaryl containing 1 nitrogen atom. In some embodiments, R³ is 5- to 6-membered heteroaryl containing 2 nitrogen atoms. In some embodiments, R³ is 5- to 6-membered heteroaryl containing 1 oxygen atom. In some embodiments, R³ is 5- to 6-membered heteroaryl containing 1 sulfur atom. In some embodiments, R³ is a 5-membered heteroaryl. In some embodiments, R³ is a 6-membered heteroaryl. In some embodiments, R³ is pyridyl, pyridazinyl, pyrimindinyl, or pyrazolyl.

In some embodiments, R³ is F. In some embodiments, R³ is Cl. In some embodiments, R³ is —OH. In some embodiments, R³ is —NH₂. In some embodiments, R³ is —NHMe. In some embodiments, R³ is —NMe₂. In some embodiments, R³ is —SMe. In some embodiments, R³ is —S(O)Me. In some embodiments, R³ is —S(O)₂Me. In some embodiments, R³ is —S(O)(═NH)Me. In some embodiments, R³ is —S(O)(═NMe)Me. In some embodiments, R³ is —CN. In some embodiments, R³ is —N02. In some embodiments, R³ is —CHF₂. In some embodiments, R³ is —CF₃. In some embodiments, R³ is —CF₂C₁. In some embodiments, R³ is —OCF₃. In some embodiments, R³ is —OCHF₂. In some embodiments, R³ is —SCF₃. In some embodiments, R³ is —SCHF₂. In some embodiments, R³ is —SF₅. In some embodiments, R³ is —P(O)(Me)₂. In some embodiments, R³ is —N₃.

In some embodiments, ring A is C₆-C₁₀ aryl optionally substituted with 1 to 5 R⁴ groups, wherein when Ring A is C₆ aryl, then any two substituents attached to adjacent atoms of said aryl are optionally taken together to form a 4- to 6-membered carbocyclic ring or a 4- to 6-membered heterocyclic ring optionally containing 1-3 heteroatoms selected from the group consisting of N, O, and S, wherein each carbocyclic or heterocyclic ring is optionally substituted with 1 to 5 R⁴ groups. In some embodiments, ring A is C₆ aryl optionally substituted with 1 to 5 R⁴ groups. In some embodiments, Ring A is C₆ aryl and two substituents attached to adjacent atoms of said aryl are taken together to form a 4- to 6-membered carbocyclic ring or a 4- to 6-membered heterocyclic ring containing 1-3 heteroatoms selected from the group consisting of N, O, and S, wherein each carbocyclic or heterocyclic ring is optionally substituted with 1 to 5 R⁴ groups. In some embodiments, the resulting 4- to 6-membered heterocyclic ring contains 1-2 heteroatoms selected from the group consisting of N and O. In some embodiments, the resulting 4- to 6-membered heterocyclic ring contains 1 nitrogen atom. In some embodiments, the resulting 4- to 6-membered heterocyclic ring contains 1 oxygen atom. In some embodiments, the resulting 4- to 6-membered heterocyclic ring contains 1 nitrogen atom and 1 oxygen atom.

In some embodiments, ring A is 5- to 10-membered heteroaryl optionally substituted with 1 to 5 R⁴ groups, and wherein when Ring A is 5- to 6-membered heteroaryl, then any two substituents attached to adjacent atoms of said heteroaryl are optionally taken together to form a 4- to 6-membered carbocyclic ring or a 4- to 6-membered heterocyclic ring optionally containing 1-3 heteroatoms selected from the group consisting of N, O, and S, wherein each carbocyclic or heterocyclic ring is optionally substituted with 1 to 5 R⁴ groups. In some embodiments, ring A is 5- to 10-membered heteroaryl optionally substituted with 1 to 5 R⁴ groups. In some embodiments, ring A is 5- to 6-membered heteroaryl wherein two substituents attached to adjacent atoms of said heteroaryl are taken together to form a 4- to 6-membered carbocyclic ring or a 4- to 6-membered heterocyclic ring optionally containing 1-3 heteroatoms selected from the group consisting of N, O, and S, wherein each carbocyclic or heterocyclic ring is optionally substituted with 1 to 5 R⁴ groups. In some embodiments, the resulting 4- to 6-membered heterocyclic ring contains 1-2 heteroatoms selected from the group consisting of N and O. In some embodiments, the resulting 4- to 6-membered heterocyclic ring contains 1 nitrogen atom. In some embodiments, the resulting 4- to 6-membered heterocyclic ring contains 1 oxygen atom. In some embodiments, the resulting 4- to 6-membered heterocyclic ring contains 1 nitrogen atom and 1 oxygen atom.

In some embodiments, Ring A is C₆ aryl or 5- to 6-membered heteroaryl, each of which is optionally substituted with 1 to 5 R⁴ groups. In some embodiments, Ring A is C₆ aryl or 5- to 6-membered heteroaryl, each of which is substituted with 1 to 5 R⁴ groups. In some embodiments, Ring A is unsubstituted C₆ aryl or unsubstituted 5- to 6-membered heteroaryl. In some embodiments, Ring A is unsubstituted phenyl or phenyl substituted with 1 to 5 R⁴ groups. In some embodiments, Ring A is 5-membered heteroaryl optionally substituted with 1 to 4 R⁴ groups. In some embodiments, Ring A is unsubstituted 5-membered heteroaryl. In some embodiments, Ring A is 6-membered heteroaryl optionally substituted with 1 to 4 R⁴ groups. In some embodiments, Ring A is unsubstituted 6-membered heteroaryl. In some embodiments, Ring A is 6-membered heteroaryl substituted with 1 to 4 R⁴ groups. In some embodiments, Ring A is pyridyl optionally substituted with 1 to 4 R⁴ groups. In some embodiments, Ring A is N

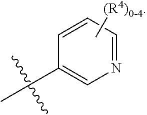

In some embodiments, Ring A is selected from the group consisting of:

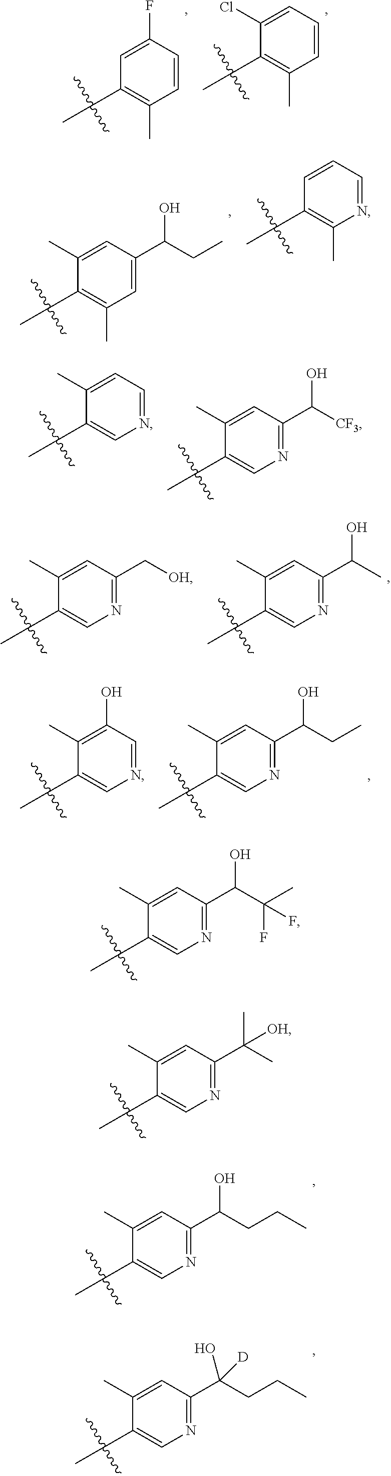

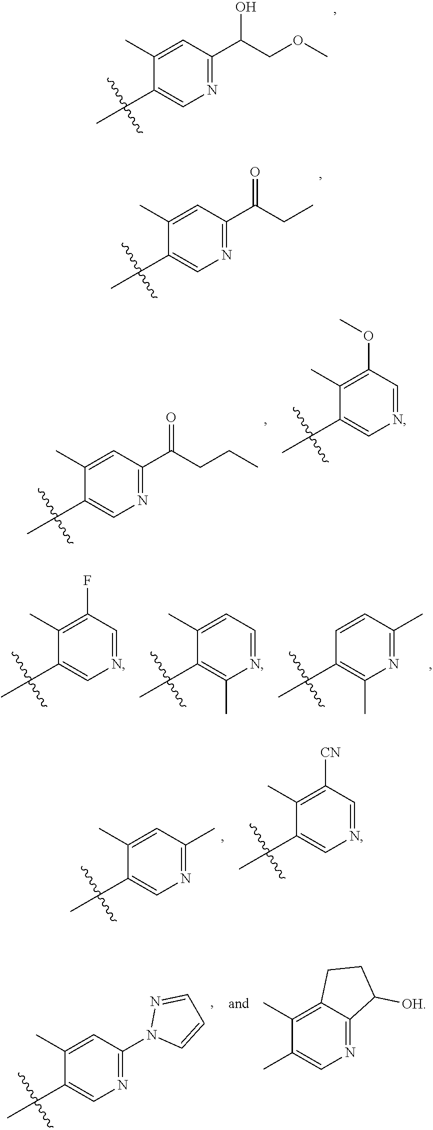

In some embodiments, Ring A is selected from the group consisting of:

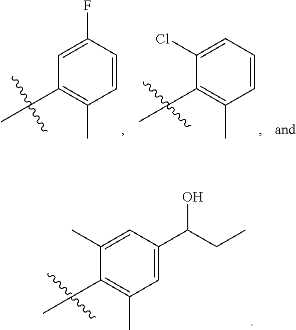

In some embodiments, Ring A is selected from the group consisting of:

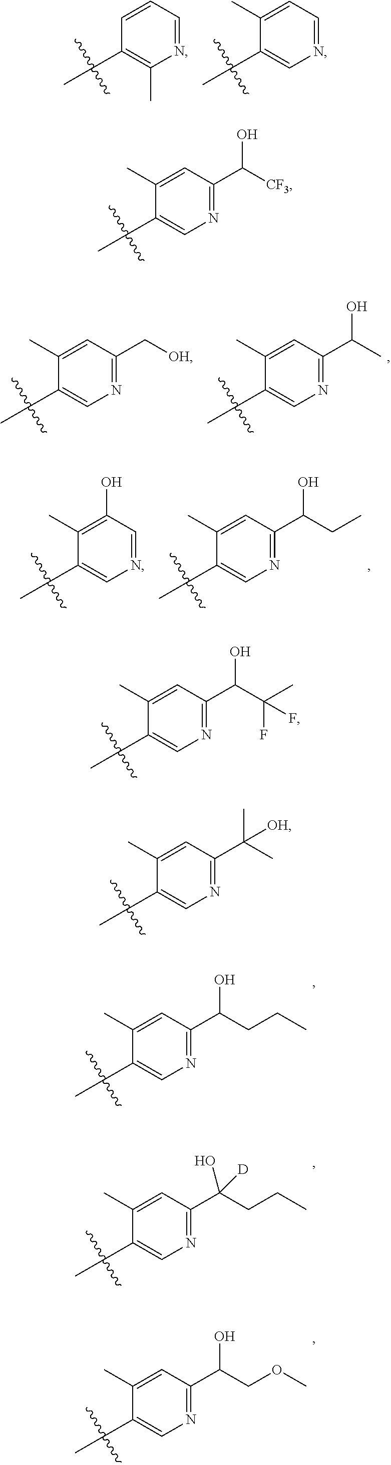

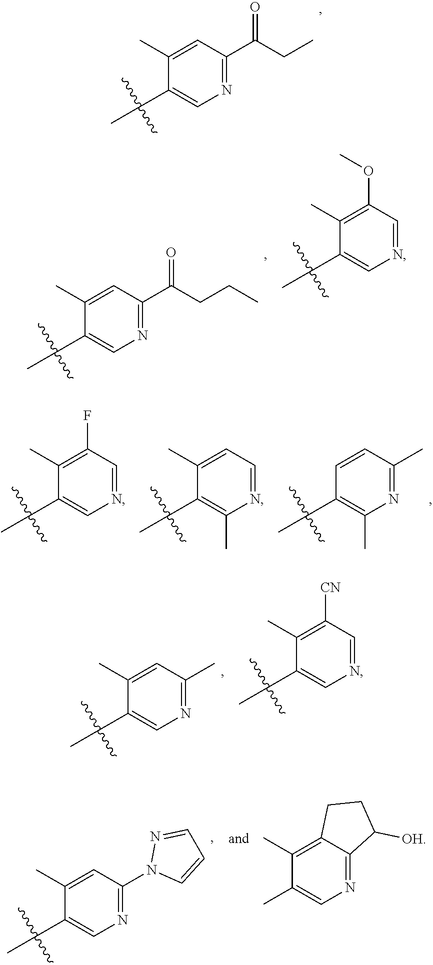

In some embodiments, Ring A is selected from the group consisting of:

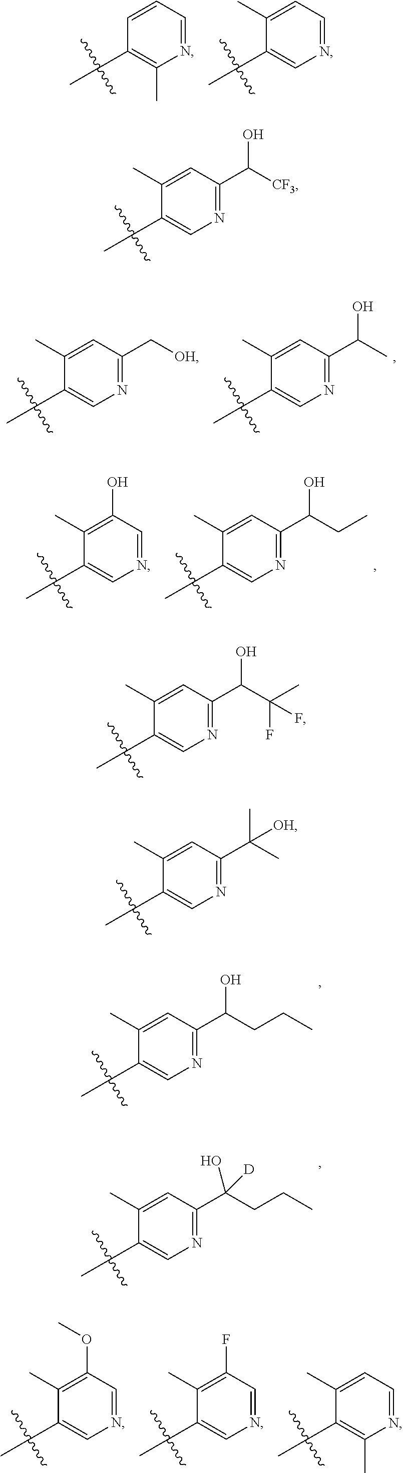

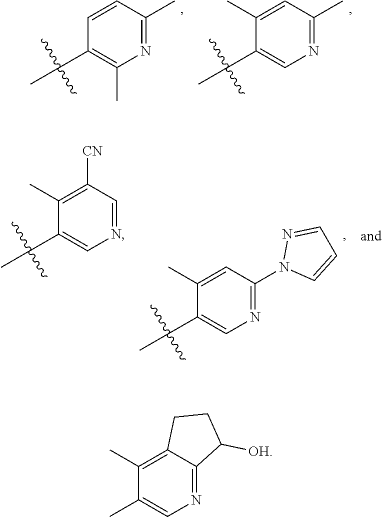

In some embodiments, Ring A is selected from the group consisting of:

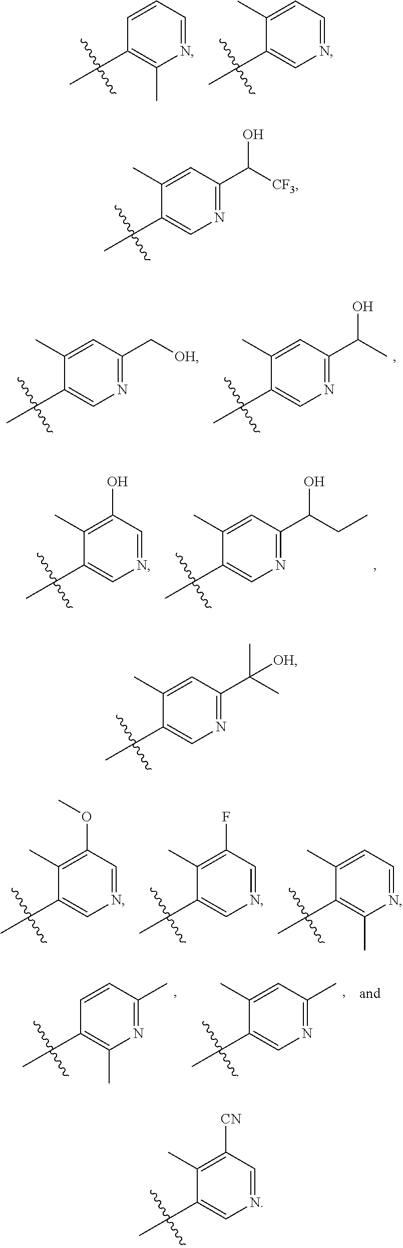

In some embodiments, each R⁴ is independently H, F, Cl, Br, I, —SCF₃, —SCHF₂, —SF₅, —OCF₃, —OR⁶, —N(R⁶)R⁶, —N₃, —OCHF₂, —CF₃, —CHF₂, —CN, —NO₂, C₁-C₆ alkyl, C₁-C₆ haloalkyl,
C₁-C₆ haloalkyl-OH, C₁-C₆ alkyl-OH, C₁-C₆ alkyl-CN, C₁-C₆ heteroalkyl, or C₃-C₇ cycloalkyl. In some embodiments, each R⁴ is independently H, F, Cl, —OCF₃, —OR⁶, —N(R⁶)R⁶, —OCHF₂,
—CF₃, —CHF₂, —CN, C₁-C₆ alkyl, C₁-C₆ haloalkyl, C₁-C₆ haloalkyl-OH, C₁-C₆ alkyl-CN, C₁-C₆ alkyl-OH, C₁-C₆ heteroalkyl, or C₃-C₇ cycloalkyl. In some embodiments, each R⁴ is independently F, C₁-C₆ alkyl, —OR⁶, —CN, —CH₂CN, —CH₂CH₂CN, C₁-C₆ alkyl-OH, or C₁-C₆ haloalkyl-OH, and each R⁶ is independently H or C₁-C₆ alkyl. In some embodiments, each R⁴ is independently F, —CH₃, —OCH₃, —OH, —CN, —CH₂OH, —CH(OH)CH₃,
—CH(OH)CH₂CH₃, —CH(OH)CF₂CH₃, —CH(OH)CH₂CH₂CH₃, —CD(OH)CH₂CH₂CH₃, —C(═O)CH₂CH₃, —C(═O)CH₂CH₂CH₃, —CH(OH)CH₂OCH₃, —C(CH₃)₂OH, or —CH(OH)CF₃. In some embodiments, each R⁴ is independently F, —CH₃, —OCH₃, —OH, —CN, —CH₂OH, —CH(OH)CH₃, —CH(OH)CH₂CH₃,
—C(CH₃)₂OH, or —CH(OH)CF₃.

In some embodiments, R⁴ is H. In some embodiments, R⁴ is F. In some embodiments, R⁴ is Cl. In some embodiments, R⁴ is Br. In some embodiments, R⁴ is I. In some embodiments, R⁴ is —SCF₃. In some embodiments, R⁴ is —SCHF₂. In some embodiments, R⁴ is
—SF₅. In some embodiments, R⁴ is —OCF₃. In some embodiments, R⁴ is —N3. In some embodiments, R⁴ is —OCHF₂. In some embodiments, R⁴ is —CF₃. In some embodiments, R⁴ is —CHF₂. In some embodiments, R⁴ is —CN. In some embodiments, R⁴ is —NO₂.

In some embodiments, R⁴ is C₁-C₆ alkyl. In some embodiments, R⁴ is C₁-C₃ alkyl. In some embodiments, R⁴ is methyl, ethyl, n-propyl, or isopropyl. In some embodiments, R⁴ is methyl.

In some embodiments, R⁴ is C₁-C₆ haloalkyl. In some embodiments, R⁴ is C₁-C₆ haloalkyl containing 1-13 halogen atoms. In some embodiments, R⁴ is C₁-C₃ haloalkyl. In some embodiments, R⁴ is C₁-C₃ haloalkyl containing 1-7 halogen atoms. In some embodiments, R⁴ is C₁-C₂ haloalkyl containing 1-5 halogen atoms. In some embodiments, the halogen atoms are independently selected from the group consisting of chloro, bromo, and fluoro atoms. In some embodiments, the halogen atoms are independently selected from the group consisting of chloro and fluoro atoms. In some embodiments, the halogen atoms are all fluoro atoms. In some embodiments, the halogen atoms are all chloro atoms. In some embodiments, the halogen atoms are a combination of chloro and fluoro atoms. In some embodiments, R⁴ is —CF₃, —CCl₃, —CF₂Cl, —CFCl₂, —CHF₂, —CH₂F, —CHC₁₂, —CH₂F, or —CHFCl. In some embodiments, R⁴ is —CF₃.

In some embodiments, R⁴ is C₁-C₆ haloalkyl-OH. In some embodiments, R⁴ is C₁-C₆ haloalkyl-OH containing 1-12 halogen atoms and one hydroxyl group. In some embodiments, R⁴ is C₁-C₆ haloalkyl-OH containing 1-12 halogen atoms and one hydroxyl group. In some embodiments, R⁴ is C₁-C₆ haloalkyl-OH containing 1-11 halogen atoms and two hydroxyl groups. In some embodiments, R⁴ is C₁-C₆ haloalkyl-OH containing 1-10 halogen atoms and three hydroxyl groups. In some embodiments, R⁴ is C₁-C₆ haloalkyl-OH containing 1-9 halogen atoms and 4 hydroxyl groups. In some embodiments, R⁴ is C₁-C₃ haloalkyl-OH. In some embodiments, R⁴ is C₁-C₃ haloalkyl-OH containing 1-6 halogen atoms and one hydroxyl group. In some embodiments, R⁴ is C₁-C₃ haloalkyl-OH containing 1-5 halogen atoms and two hydroxyl groups. In some embodiments, R⁴ is C₁-C₃ haloalkyl-OH containing 1-4 halogen atoms and three hydroxyl groups. In some embodiments, the halogen atoms are independently selected from the group consisting of chloro, bromo, and fluoro atoms. In some embodiments, the halogen atoms are independently selected from the group consisting of chloro and fluoro atoms. In some embodiments, the halogen atoms are all fluoro atoms. In some embodiments, the halogen atoms are all chloro atoms. In some embodiments, the halogen atoms are a combination of chloro and fluoro atoms. In some embodiments, R⁴ is —CH(OH)CF₃, —CHF(OH)CH₃,
—CF₂(OH)CH₃, or —CF₂(OH)CF₃. In some embodiments, R⁴ is —CH(OH)CF₃.

In some embodiments, R⁴ is C₁-C₆ alkyl-OH. In some embodiments, R⁴ is C₁-C₆ alkyl-OH containing 1-6 hydroxyl groups. In some embodiments, R⁴ is C₁-C₆ alkyl-OH containing 1 hydroxyl group. In some embodiments, R⁴ is C₁-C₆ alkyl-OH containing 2 hydroxyl groups. In some embodiments, R⁴ is C₁-C₆ alkyl-OH containing 3 hydroxyl groups. In some embodiments, R⁴ is C₁-C₃ alkyl-OH. In some embodiments, R⁴ is C₁-C₃ alkyl-OH containing 1-3 hydroxyl groups. In some embodiments, R⁴ is C₁-C₃ alkyl-OH containing 1 hydroxyl group. In some embodiments, R⁴ is —CH₂OH, —CH₂CH₂OH, —CH(OH)CH₃, —CH₂CH₂CH₂OH, —CH(OH)CH₂CH₃, —CH(OH)CH₂CH₂CH₃, —CH₂CH(OH)CH₃, or —C(CH₃)₂OH. In some embodiments, R⁴ is —CH₂OH, —CH(OH)CH₃, —CH(OH)CH₂CH₃, or —C(CH₃)₂OH.

In some embodiments, R⁴ is C₁-C₆ alkyl-OR⁶. In some embodiments, R⁴ is C₁-C₆ alkyl-OR⁶ containing 1-6 —OR⁶ groups. In some embodiments, R⁴ is C₁-C₆ alkyl-OR⁶ containing 1 —OR⁶ group. In some embodiments, R⁴ is C₁-C₆ alkyl-OR⁶ containing 2 —OR⁶ groups. In some embodiments, R⁴ is C₁-C₆ alkyl-OR⁶ containing 3 —OR⁶ groups. In some embodiments, R⁴ is C₁-C₃ alkyl-OR⁶. In some embodiments, R⁴ is C₁-C₃ alkyl-OR⁶ containing 1-3 —OR⁶ groups. In some embodiments, R⁴ is C₁-C₃ alkyl-OR⁶ containing 1 —OR⁶ group. In some embodiments, R⁴ is —CH₂—OR⁶, —CH₂CH₂—OR⁶, —CH(—OR⁶)CH₃, —CH₂CH₂CH₂—OR⁶, —CH(—OR⁶)CH₂CH₃, —CH₂CH(—OR⁶)CH₃, or —C(CH₃)₂—OR⁶. In some embodiments, R⁴ is —CH₂—OR⁶, —CH(—OR⁶)CH₃, —CH(—OR⁶)CH₂CH₃, or —C(CH₃)₂—OR⁶.

In some embodiments, R⁴ is C₁-C₆ alkylene-C₁-C₃ alkoxy. In some embodiments, R⁴ is C₁-C₃ alkylene-C₁-C₃ alkoxy. In some embodiments, R⁴ is —CH₂OCH₃, —CH₂CH₂OCH₃, —CH₂OCH₂CH₃, or —CH₂CH₂OCH₂CH₃.

In some embodiments, R⁴ is C₁-C₆ alkylene(OH)—C₁-C₃ alkoxy. In some embodiments, R⁴ is C₁-C₃ alkylene(OH)—C₁-C₃ alkoxy. In some embodiments, R⁴ is —CH(OH)CH₂OCH₃, —CH₂CH(OH)OCH₃, —CH(OH)CH₂OCH₂CH₃, or —CH₂CH(OH)OCH₂CH₃.

In some embodiments, R⁴ is C₁-C₆ alkyl-CN. In some embodiments, R⁴ is C₁-C₆ alkyl-CN containing 1-6 cyano groups. In some embodiments, R⁴ is C₁-C₆ alkyl-CN containing 1 cyano group. In some embodiments, R⁴ is C₁-C₆ alkyl-CN containing 2 cyano groups. In some embodiments, R⁴ is C₁-C₆ alkyl-CN containing 3 cyano groups. In some embodiments, R⁴ is C₁-C₃ alkyl-CN. In some embodiments, R⁴ is C₁-C₃ alkyl-CN containing 1-3 cyano groups. In some embodiments, R⁴ is C₁-C₃ alkyl-CN containing 1 cyano group. In some embodiments, R⁴ is —CH₂CN, —CH₂CH₂CN, —CH(CN)CH₃, —CH₂CH₂CH₂CN, —CH(CN)CH₂CH₃, or
—CH₂CH(CN)CH₃. In some embodiments, R⁴ is —CH₂CN or —CH₂CH₂CN. In some embodiments, R⁴ is —CH₂CN. In some embodiments, R⁴ is —CH₂CH₂CN.

In some embodiments, R⁴ is C₁-C₆ heteroalkyl. In some embodiments, R⁴ is C₁-C₆ heteroalkyl containing 1-3 heteroatoms selected from the group consisting of N and O. In some embodiments, R⁴ is C₁-C₆ heteroalkyl containing 1 nitrogen atom. In some embodiments, R⁴ is C₁-C₆ heteroalkyl containing 1 oxygen atom. In some embodiments, R⁴ is C₁-C₃ heteroalkyl. In some embodiments, R⁴ is —CH₂—CH₂—O—CH₃, —CH₂—O—CH₃, —CH₂—CH₂—NH—CH₃, or —CH₂—NH—CH₃.

In some embodiments, R⁴ is C₃-C₇ cycloalkyl. In some embodiments, R⁴ is C₃-C₆ cycloalkyl. In some embodiments, R⁴ is cyclopropyl, cyclobutyl, cyclopentyl, or cyclohexyl.

In some embodiments, R⁴ is C₁-C₆ alkylene-C₃-C₇ cycloalkyl. In some embodiments, R⁴ is C₁-C₃ alkylene-C₃-C₆ cycloalkyl. In some embodiments, R⁴ is —CH₂-cyclopropyl, —CH₂CH₂-cyclopropyl, —CH₂-cyclobutyl, —CH₂CH₂-cyclobutyl, —CH₂-cyclopentyl, —CH₂CH₂-cyclopentyl, —CH₂-cyclohexyl, or —CH₂CH₂-cyclohexyl.

In some embodiments, R⁴ is C₁-C₆ alkylene(—OH)—C₃-C₇ cycloalkyl. In some embodiments, R⁴ is C₁-C₃ alkylene(—OH)—C₃-C₆ cycloalkyl. In some embodiments, R⁴ is —CH(OH)-cyclopropyl, —CH(OH)CH₂-cyclopropyl, —CH(OH)-cyclobutyl, —CH₂CH₂-cyclobutyl, —CH(OH)-cyclopentyl, —CH(OH)CH₂-cyclopentyl, —CH(OH)-cyclohexyl, or —CH(OH)CH₂-cyclohexyl.

In some embodiments, R⁴ is —OR⁶. In some embodiments, R⁴ is —OR⁶, and R⁶ is H or C₁-C₆ alkyl. In some embodiments, R⁴ is —OR⁶, and R⁶ is H or C₁-C₃ alkyl. In some embodiments, R⁴ is —OR⁶, and R⁶ is H or methyl. In some embodiments, R⁴ is —OH. In some embodiments, R⁴ is —O(C₁-C₆ alkyl). In some embodiments, R⁴ is —OCH₃.

In some embodiments, R⁴ is —N(R⁶)R⁶. In some embodiments where R⁴ is —N(R⁶)R⁶, each R⁶ is the same. In other embodiments where R⁴ is —N(R⁶)R⁶, each R⁶ is different.

In any of the embodiments described herein, R⁶ is independently H, C₁-C₆ alkyl, C₁-C₆ haloalkyl, C₁-C₆ heteroalkyl, C₃-C₇ cycloalkyl, C₆ aryl, 5- to 10-membered heteroaryl, or 4- to 7-membered heterocycloalkyl. In some embodiments, R⁶ is independently H, C₁-C₆ alkyl, C₁-C₆ haloalkyl, C₁-C₆ heteroalkyl, or C₃-C₇ cycloalkyl.

In some embodiments, R⁶ is H.

In some embodiments, R⁶ is C₁-C₆ alkyl. In some embodiments, R⁶ is C₁-C₃ alkyl. In some embodiments, R⁶ is methyl, ethyl, n-propyl, or isopropyl. In some embodiments, R⁶ is methyl.

In some embodiments, R⁶ is C₁-C₆ haloalkyl. In some embodiments, R⁶ is C₁-C₆ haloalkyl containing 1-13 halogen atoms. In some embodiments, R⁶ is C₁-C₃ haloalkyl. In some embodiments, R⁶ is C₁-C₃ haloalkyl containing 1-7 halogen atoms. In some embodiments, R⁶ is C₁-C₂ haloalkyl containing 1-5 halogen atoms. In some embodiments, the halogen atoms are independently selected from the group consisting of chloro, bromo, and fluoro atoms. In some embodiments, the halogen atoms are independently selected from the group consisting of chloro and fluoro atoms. In some embodiments, the halogen atoms are all fluoro atoms. In some embodiments, the halogen atoms are all chloro atoms. In some embodiments, the halogen atoms are a combination of chloro and fluoro atoms. In some embodiments, R⁶ is —CF₃, —CCl₃, —CF₂Cl, —CFCl₂, —CHF₂, —CH₂F, —CHC₁₂, —CH₂F, or —CHFCl. In some embodiments, R⁶ is —CF₃.

In some embodiments, R⁶ is C₁-C₆ heteroalkyl. In some embodiments, R⁶ is C₁-C₆ heteroalkyl containing 1-3 heteroatoms selected from the group consisting of N and O. In some embodiments, R⁶ is C₁-C₆ heteroalkyl containing 1 nitrogen atom. In some embodiments, R⁶ is C₁-C₆ heteroalkyl containing 1 oxygen atom. In some embodiments, R⁶ is C₁-C₃ heteroalkyl. In some embodiments, R⁶ is —CH₂—CH₂—O—CH₃, —CH₂—O—CH₃, —CH₂—CH₂—NH—CH₃, or —CH₂—NH—CH₃.

In some embodiments, R⁶ is C₃-C₇ cycloalkyl. In some embodiments, R⁶ is C₃-C₆ cycloalkyl. In some embodiments, R⁶ is cyclopropyl, cyclobutyl, cyclopentyl, or cyclohexyl.

In some embodiments, R⁶ is C₆ aryl. In some embodiments, R⁶ is phenyl.

In some embodiments, R⁶ is 5- to 10-membered heteroaryl. In some embodiments, R⁶ is 5- to 10-membered heteroaryl containing 1-3 heteroatoms selected from the group consisting of O, N, and S. In some embodiments, R⁶ is 5- to 10-membered heteroaryl containing 1-2 heteroatoms selected from the group consisting of O and N. In some embodiments, R⁶ is 5- to 10-membered heteroaryl containing 1 nitrogen atom. In some embodiments, R⁶ is 5- to 10-membered heteroaryl containing 1 oxygen atom. In some embodiments, R⁶ is 5- to 10-membered heteroaryl containing 2 nitrogen atoms. In some embodiments, R⁶ is 5- to 10-membered heteroaryl containing 1 nitrogen atom and 1 oxygen atom. In some embodiments, R⁶ is 8- to 10-membered heteroaryl. In some embodiments, R⁶ is 8-membered heteroaryl. In some embodiments, R⁶ is 10-membered heteroaryl. In some embodiments, R⁶ is 5- to 6-membered heteroaryl. In some embodiments, R⁶ is 5-membered heteroaryl. In some embodiments, R⁶ is 6-membered heteroaryl. In some embodiments, R⁶ is pyridyl, pyridazinyl, pyrimindinyl, or pyrazolyl.

In some embodiments, R⁶ is 4- to 7-membered heterocycloalkyl. In some embodiments, R⁶ is 4- to 7-membered heterocycloalkyl containing 1-3 heteroatoms selected from the group consisting of N and O. In some embodiments, R⁶ is 4- to 7-membered heterocycloalkyl containing 1-2 nitrogen atoms. In some embodiments, R⁶ is 4- to 7-membered heterocycloalkyl containing 1-2 oxygen atoms. In some embodiments, R⁶ is 4- to 7-membered heterocycloalkyl containing 1 oxygen atom and 1 nitrogen atom. In some embodiments, R⁶ is 4- to 7-membered heterocycloalkyl containing 1 nitrogen atom. In some embodiments, R⁶ is 4- to 7-membered heterocycloalkyl containing 1 oxygen atom. In some embodiments, R⁶ is 4- to 7-membered heterocycloalkyl containing 2 nitrogen atoms. In some embodiments, R⁶ is 4- to 6-membered heterocycloalkyl. In some embodiments, R⁶ is 5- to 6-membered heterocycloalkyl. In some embodiments, R⁶ is pyrrolidinyl or piperidinyl.

In some embodiments, each R⁵ is independently F, Cl, —SCF₃, —SCHF₂, —SF₅, —OCF₃, —OR⁶, —N(R⁶)R⁶, —N₃, —OCHF₂, —CF₃, —CF₂Cl, —CHF₂, —CN, —NO₂, C₁-C₆ alkyl, C₁-C₆ haloalkyl, C₁-C₆ heteroalkyl, or C₃-C₇ cycloalkyl. In some embodiments, each R⁵ is independently F, —OCF₃, —OR⁶, —N(R⁶)R⁶, —OCHF₂, —CF₃, —CN, C₁-C₆ alkyl, C₁-C₆ haloalkyl, or C₁-C₆ heteroalkyl. In some embodiments, each R⁵ is F.

In some embodiments, R⁵ is F. In some embodiments, R⁵ is Cl. In some embodiments, R⁵ is —SCF₃. In some embodiments, R⁵ is —SCHF₂. In some embodiments, R⁵ is
—SF₅. In some embodiments, R⁵ is —OCF₃. In some embodiments, R⁵ is —N3. In some embodiments, R⁵ is —OCHF₂. In some embodiments, R⁵ is —CF₃. In some embodiments, R⁵ is —CF₂C₁. In some embodiments, R⁵ is —CHF₂. In some embodiments, R⁵ is —CN. In some embodiments, R⁵ is —NO₂.

In some embodiments, R⁵ is —OR⁶. In some embodiments, R⁵ is —OR⁶, and R⁶ is H or C₁-C₆ alkyl. In some embodiments, R⁵ is —OR⁶, and R⁶ is H or C₁-C₃ alkyl. In some embodiments, R⁵ is —OR⁶, and R⁶ is H or methyl. In some embodiments, R⁵ is —OH. In some embodiments, R⁵ is —O(C₁-C₆ alkyl). In some embodiments, R⁵ is —OCH₃.

In some embodiments, R⁵ is —N(R⁶)R⁶. In some embodiments where R⁵ is —N(R⁶)R⁶, each R⁶ is the same. In other embodiments where R⁵ is —N(R⁶)R⁶, each R⁶ is different.

In some embodiments, R⁵ is C₁-C₆ alkyl. In some embodiments, R⁵ is C₁-C₃ alkyl. In some embodiments, R⁵ is methyl, ethyl, n-propyl, or isopropyl. In some embodiments, R⁵ is methyl.

In some embodiments, R⁵ is C₁-C₆ haloalkyl. In some embodiments, R⁵ is C₁-C₆ haloalkyl containing 1-13 halogen atoms. In some embodiments, R⁵ is C₁-C₃ haloalkyl. In some embodiments, R⁵ is C₁-C₃ haloalkyl containing 1-7 halogen atoms. In some embodiments, R⁵ is C₁-C₂ haloalkyl containing 1-5 halogen atoms. In some embodiments, the halogen atoms are independently selected from the group consisting of chloro, bromo, and fluoro atoms. In some embodiments, the halogen atoms are independently selected from the group consisting of chloro and fluoro atoms. In some embodiments, the halogen atoms are all fluoro atoms. In some embodiments, the halogen atoms are all chloro atoms. In some embodiments, the halogen atoms are a combination of chloro and fluoro atoms. In some embodiments, R⁵ is —CF₃, —CCl₃, —CF₂Cl, —CFCl₂, —CHF₂, —CH₂F, —CHCl₂, —CH₂F, or —CHFCl. In some embodiments, R⁵ is —CF₃.

In some embodiments, R⁵ is C₁-C₆ heteroalkyl. In some embodiments, R⁵ is C₁-C₆ heteroalkyl containing 1-3 heteroatoms selected from the group consisting of N and O. In some embodiments, R⁵ is C₁-C₆ heteroalkyl containing 1 nitrogen atom. In some embodiments, R⁵ is C₁-C₆ heteroalkyl containing 1 oxygen atom. In some embodiments, R⁵ is C₁-C₃ heteroalkyl. In some embodiments, R⁵ is —CH₂—CH₂—O—CH₃, —CH₂—CH₂—OH, —CH₂—O—CH₃, —CH₂—CH₂—NH—CH₃,
—CH₂—CH₂—N(CH₃)₂, —CH₂—N(CH₃)₂, or —CH₂—NH—CH₃.

In some embodiments, R⁵ is C₃-C₇ cycloalkyl. In some embodiments, R⁵ is C₃-C₆ cycloalkyl. In some embodiments, R⁵ is cyclopropyl, cyclobutyl, cyclopentyl, or cyclohexyl.

In some embodiments, the moiety

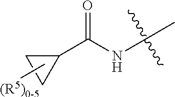

of formula (I) is

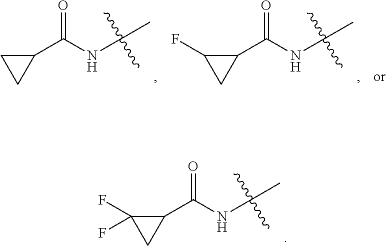 [SMILES: *NC(=O)C1CC1 ; *NC(=O)C1CC1F ; *NC(=O)C1CC1(F)F]

In some variations, R⁵ is absent. In other variations, 1-5 R⁵ groups are present. In some embodiments, 1 or 2 R⁵ groups are present.

In some embodiments, the compound of formula (I) is of formula (IA), (IB), (IC), (ID), (IE), (IF), or (IG):

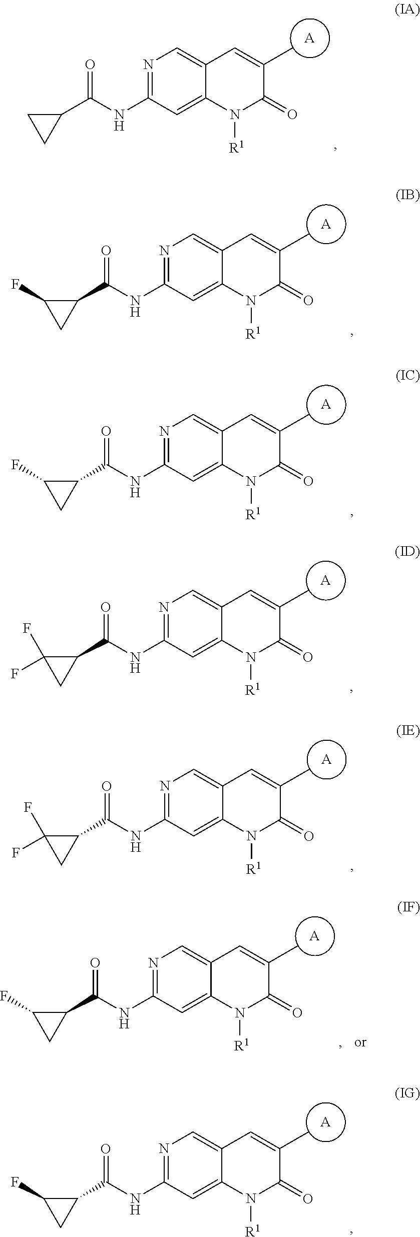

wherein Ring A and R¹ are as defined for formula (I). In some embodiments, the compound is of formula (IA). In some embodiments, the compound is of formula (IB). In some embodiments, the compound is of formula (IC). In some embodiments, the compound is of formula (ID). In some embodiments, the compound is of formula (IE). In some embodiments, the compound is of formula (IF). In some embodiments, the compound is of formula (IG). In any variation of formula (IA), (IB), (IC), (ID), (IE), (IF), or (IG), R¹ is H, C₁-C₆ alkyl, or C₁-C₆ alkyl-CN; and Ring A is 5- to 10-membered heteroaryl optionally substituted with 1 to 5 R⁴ groups. In some embodiments, R¹ is H, methyl, ethyl, or —CH₂-cyclopropyl. In some embodiments, R¹ is methyl. In some embodiments, Ring A is 6-membered heteroaryl optionally substituted with 1 to 4 R⁴ groups. In some embodiments, Ring A is pyridyl optionally substituted with 1 to 4 R⁴ groups. In some embodiments, each R⁴ is independently F, C₁-C₆ alkyl, —OR⁶, —CN, C₁-C₆ alkyl-OH, or C₁-C₆ haloalkyl-OH; and each R⁶ is independently H or C₁-C₆ alkyl. In some embodiments, each R⁴ is independently F, —CH₃, —OCH₃, —OH, —CN, —CH₂OH, —CH(OH)CH₃,
—CH(OH)CH₂CH₃, —C(CH₃)₂OH, —CH₂CN, —CH₂CH₂CN, or —CH(OH)CF₃.

In some embodiments, the compound of formula (I) is of formula (I-a), (I-b), (I-c), (I-d), (I-e), (I-f), or (I-g).

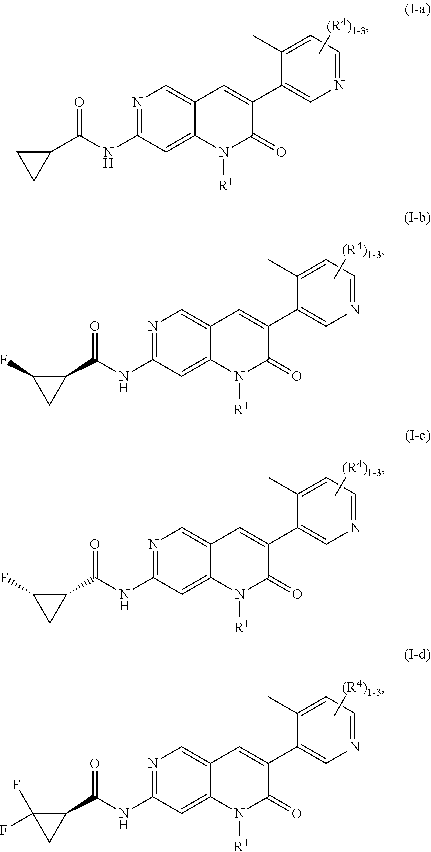

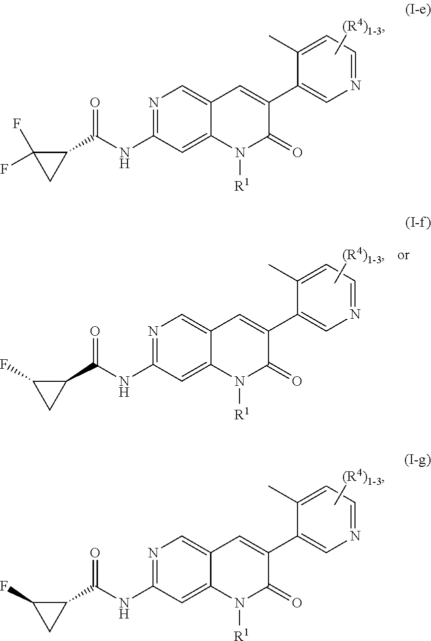

wherein R¹ and R⁴ are as described for formula (I). In some embodiments, the compound is of formula (I-a). In some embodiments, the compound is of formula (I-b). In some embodiments, the compound is of formula (I-c). In some embodiments, the compound is of formula (I-d). In some embodiments, the compound is of formula (I-e). In some embodiments, the compound is of formula (I-f). In some embodiments, the compound is of formula (I-g). In any variation of formula (I-a), (I-b), (I-c), (I-d), (I-e), (I-f), or (I-g), R¹ is H, C₁-C₆ alkyl, or C₁-C₆ alkyl-CN; each R⁴ is independently F, —OR⁶, —CN, C₁-C₆ alkyl, C₁-C₆ alkyl-OH, C₁-C₆ alkyl-CN, or C₁-C₆ haloalkyl-OH; and each R⁶ is independently H or C₁-C₆ alkyl. In some embodiments, R¹ is H or —CH₃; and each R⁴ is independently H, F, —CH(OH)CH₃, —CH(OH)CH₂CH₃,
—CH(OH)CH₂CH₂CH₃, —CD(OH)CH₂CH₂CH₃, —C(CH₃)₂OH, or —CH(OH)CF₃. In some embodiments, R¹ is —CH₃; and each R⁴ is independently F, —CH₃, —OCH₃, —OH, —CN, —CH₂OH, —CH(OH)CH₃, —CH(OH)CH₂CH₃, —C(CH₃)₂OH, —CH₂CN, —CH₂CH₂CN, or —CH(OH)CF₃.

In some embodiments, provided is a compound selected from the compounds in Table 1, or a pharmaceutically acceptable salt, solvate, hydrate, or co-crystal thereof, or a mixture of any of the foregoing.

TABLE 1

| Cmpd No. | Structure | Cmpd No. | Structure |
|---|---|---|---|
| 1 | 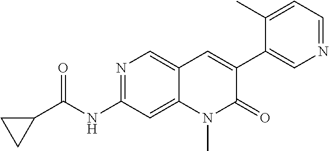 [SMILES: Cc1ccncc1-c1cc2cnc(NC(=O)C3CC3)cc2n(C)c1=O] | | |
| 2 | 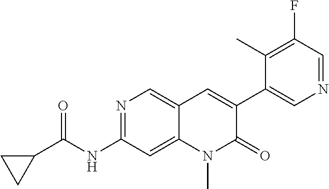 [SMILES: Cc1c(F)cncc1-c1cc2cnc(NC(=O)C3CC3)cc2n(C)c1=O] | | |
| 3 | 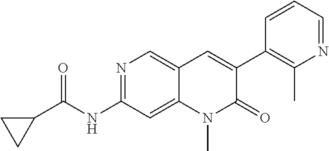 [SMILES: Cc1ncccc1-c1cc2cnc(NC(=O)C3CC3)cc2n(C)c1=O] | | |
| 4 | 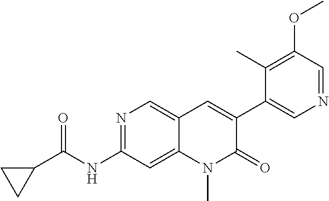 [SMILES: COc1cncc(-c2cc3cnc(NC(=O)C4CC4)cc3n(C)c2=O)c1C] | | |
| 5 | 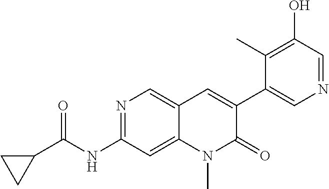 [SMILES: Cc1c(O)cncc1-c1cc2cnc(NC(=O)C3CC3)cc2n(C)c1=O] | | |
| 6 | 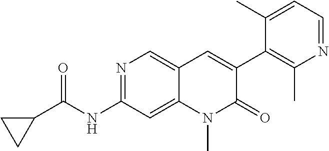 [SMILES: Cc1ccnc(C)c1-c1cc2cnc(NC(=O)C3CC3)cc2n(C)c1=O] | | |
| 7 | 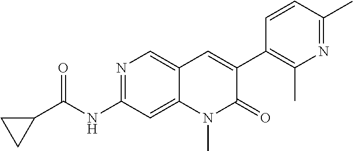 [SMILES: Cc1ccc(-c2cc3cnc(NC(=O)C4CC4)cc3n(C)c2=O)c(C)n1] | | |
| 8 | 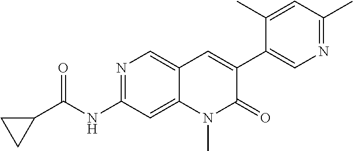 [SMILES: Cc1cc(C)c(-c2cc3cnc(NC(=O)C4CC4)cc3n(C)c2=O)cn1] | | |
| 9 | 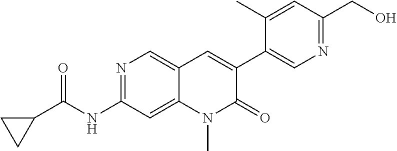 [SMILES: Cc1cc(CO)ncc1-c1cc2cnc(NC(=O)C3CC3)cc2n(C)c1=O] | | |
| 10 | 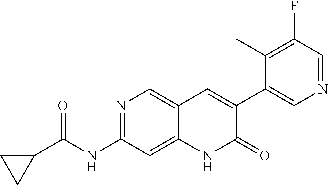 [SMILES: Cc1c(F)cncc1-c1cc2cnc(NC(=O)C3CC3)cc2[nH]c1=O] | | |
| 11 | 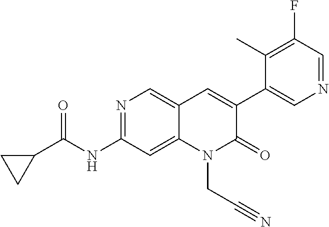 [SMILES: Cc1c(F)cncc1-c1cc2cnc(NC(=O)C3CC3)cc2n(CC#N)c1=O] | | |
| 12 | 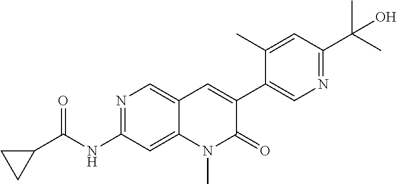 [SMILES: Cc1cc(C(C)(C)O)ncc1-c1cc2cnc(NC(=O)C3CC3)cc2n(C)c1=O] | | |
| 13 | 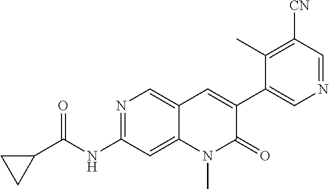 [SMILES: Cc1c(C#N)cncc1-c1cc2cnc(NC(=O)C3CC3)cc2n(C)c1=O] | | |
| 14 | 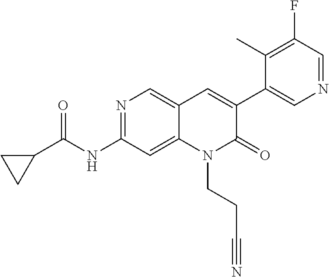 [SMILES: Cc1c(F)cncc1-c1cc2cnc(NC(=O)C3CC3)cc2n(CCC#N)c1=O] | | |
| 15 | 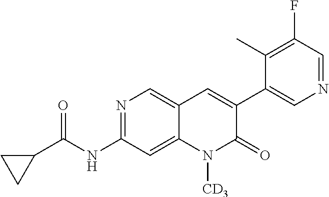 [SMILES: [2H]C([2H])([2H])n1c(=O)c(-c2cncc(F)c2C)cc2cnc(NC(=O)C3CC3)cc21] | | |
| 16 | 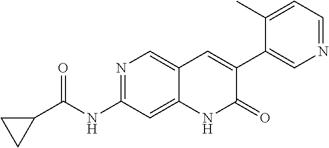 [SMILES: Cc1ccncc1-c1cc2cnc(NC(=O)C3CC3)cc2[nH]c1=O] | | |
| 17 | 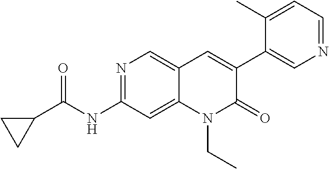 [SMILES: CCn1c(=O)c(-c2cnccc2C)cc2cnc(NC(=O)C3CC3)cc21] | | |
| 18 | 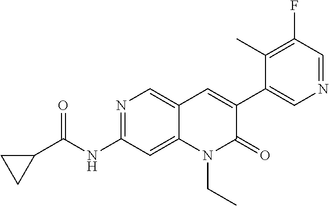 [SMILES: CCn1c(=O)c(-c2cncc(F)c2C)cc2cnc(NC(=O)C3CC3)cc21] | | |
| 19 | 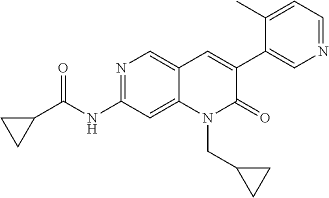 [SMILES: Cc1ccncc1-c1cc2cnc(NC(=O)C3CC3)cc2n(CC2CC2)c1=O] | | |
| 20 | 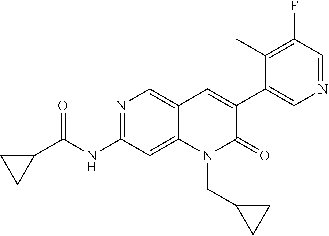 [SMILES: Cc1c(F)cncc1-c1cc2cnc(NC(=O)C3CC3)cc2n(CC2CC2)c1=O] | | |
| 21 | 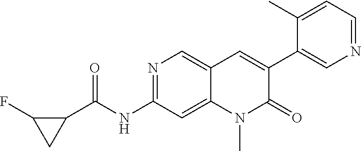 [SMILES: Cc1ccncc1-c1cc2cnc(NC(=O)C3CC3F)cc2n(C)c1=O] | | |
| 22 | 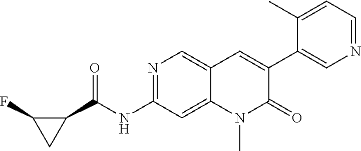 [SMILES: Cc1ccncc1-c1cc2cnc(NC(=O)[C@H]3C[C@H]3F)cc2n(C)c1=O] | 21 | 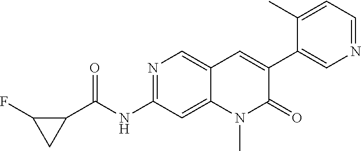 [SMILES: Cc1ccncc1-c1cc2cnc(NC(=O)C3CC3F)cc2n(C)c1=O] |
| 23 | 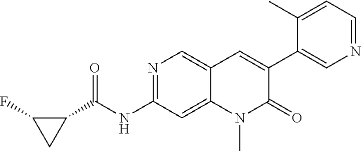 [SMILES: Cc1ccncc1-c1cc2cnc(NC(=O)[C@@H]3C[C@@H]3F)cc2n(C)c1=O] | 21 | 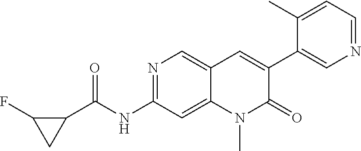 [SMILES: Cc1ccncc1-c1cc2cnc(NC(=O)C3CC3F)cc2n(C)c1=O] |
| 24 | 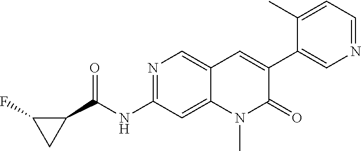 [SMILES: Cc1ccncc1-c1cc2cnc(NC(=O)[C@H]3C[C@@H]3F)cc2n(C)c1=O] | 21 | 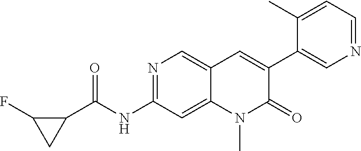 [SMILES: Cc1ccncc1-c1cc2cnc(NC(=O)C3CC3F)cc2n(C)c1=O] |
| 25 | 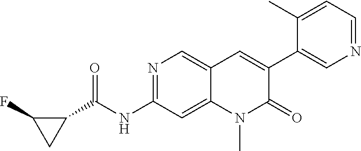 [SMILES: Cc1ccncc1-c1cc2cnc(NC(=O)[C@@H]3C[C@H]3F)cc2n(C)c1=O] | 21 | 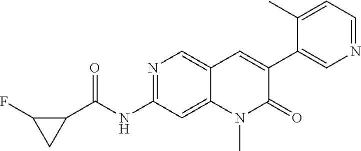 [SMILES: Cc1ccncc1-c1cc2cnc(NC(=O)C3CC3F)cc2n(C)c1=O] |
| 26 | 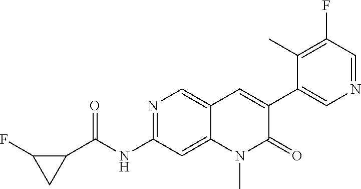 [SMILES: Cc1c(F)cncc1-c1cc2cnc(NC(=O)C3CC3F)cc2n(C)c1=O] | | |
| 27 | 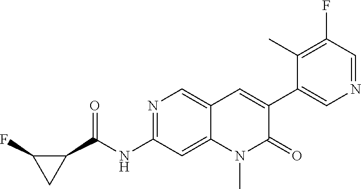 [SMILES: Cc1c(F)cncc1-c1cc2cnc(NC(=O)[C@H]3C[C@H]3F)cc2n(C)c1=O] | 26 | 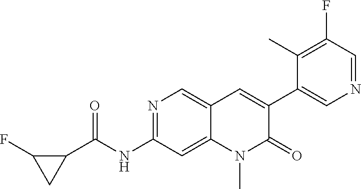 [SMILES: Cc1c(F)cncc1-c1cc2cnc(NC(=O)C3CC3F)cc2n(C)c1=O] |
| 28 | 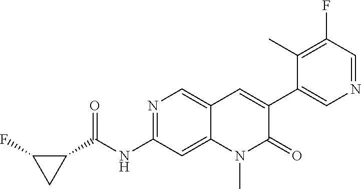 [SMILES: Cc1c(F)cncc1-c1cc2cnc(NC(=O)[C@@H]3C[C@@H]3F)cc2n(C)c1=O] | 26 | 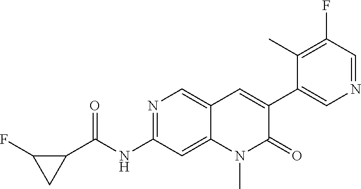 [SMILES: Cc1c(F)cncc1-c1cc2cnc(NC(=O)C3CC3F)cc2n(C)c1=O] |
| 29 | 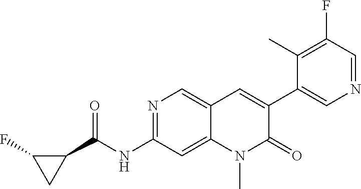 [SMILES: Cc1c(F)cncc1-c1cc2cnc(NC(=O)[C@H]3C[C@@H]3F)cc2n(C)c1=O] | 26 | 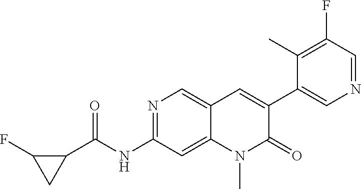 [SMILES: Cc1c(F)cncc1-c1cc2cnc(NC(=O)C3CC3F)cc2n(C)c1=O] |
| 30 | 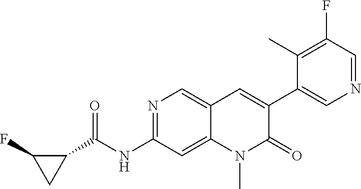 [SMILES: Cc1c(F)cncc1-c1cc2cnc(NC(=O)[C@@H]3C[C@H]3F)cc2n(C)c1=O] | 26 | 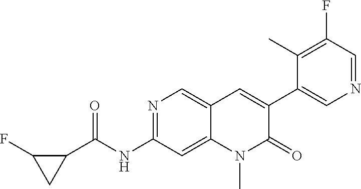 [SMILES: Cc1c(F)cncc1-c1cc2cnc(NC(=O)C3CC3F)cc2n(C)c1=O] |
| 31 | 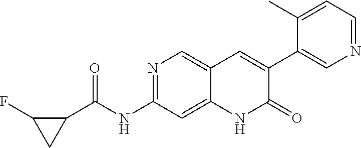 [SMILES: Cc1ccncc1-c1cc2cnc(NC(=O)C3CC3F)cc2[nH]c1=O] | | |
| 32 | 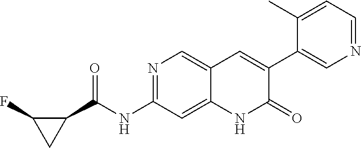 [SMILES: Cc1ccncc1-c1cc2cnc(NC(=O)[C@H]3C[C@H]3F)cc2[nH]c1=O] | 31 | 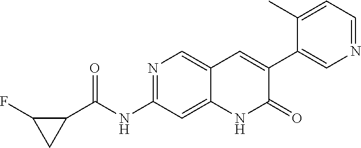 [SMILES: Cc1ccncc1-c1cc2cnc(NC(=O)C3CC3F)cc2[nH]c1=O] |
| 33 | 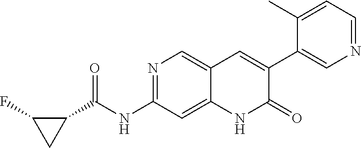 [SMILES: Cc1ccncc1-c1cc2cnc(NC(=O)[C@@H]3C[C@@H]3F)cc2[nH]c1=O] | 31 | 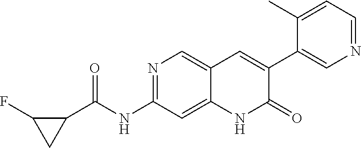 [SMILES: Cc1ccncc1-c1cc2cnc(NC(=O)C3CC3F)cc2[nH]c1=O] |
| 34 | 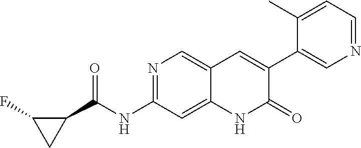 [SMILES: Cc1ccncc1-c1cc2cnc(NC(=O)[C@H]3C[C@@H]3F)cc2[nH]c1=O] | 31 | 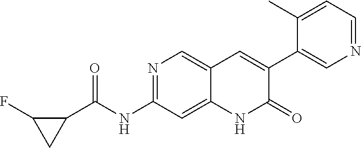 [SMILES: Cc1ccncc1-c1cc2cnc(NC(=O)C3CC3F)cc2[nH]c1=O] |
| 35 | 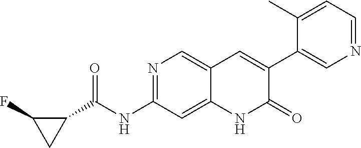 [SMILES: Cc1ccncc1-c1cc2cnc(NC(=O)[C@@H]3C[C@H]3F)cc2[nH]c1=O] | 31 | 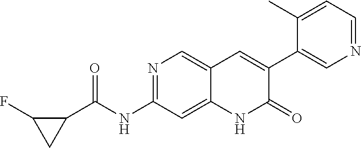 [SMILES: Cc1ccncc1-c1cc2cnc(NC(=O)C3CC3F)cc2[nH]c1=O] |
| 36 | 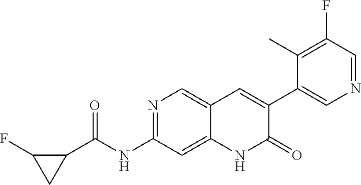 [SMILES: Cc1c(F)cncc1-c1cc2cnc(NC(=O)C3CC3F)cc2[nH]c1=O] | | |
| 37 | 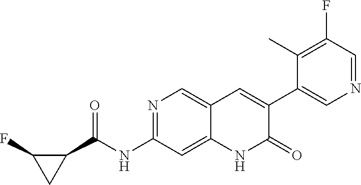 [SMILES: Cc1c(F)cncc1-c1cc2cnc(NC(=O)[C@H]3C[C@H]3F)cc2[nH]c1=O] | 36 | 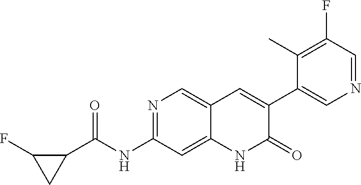 [SMILES: Cc1c(F)cncc1-c1cc2cnc(NC(=O)C3CC3F)cc2[nH]c1=O] |
| 38 | 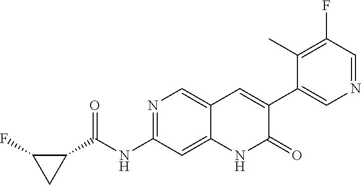 [SMILES: Cc1c(F)cncc1-c1cc2cnc(NC(=O)[C@@H]3C[C@@H]3F)cc2[nH]c1=O] | 36 | 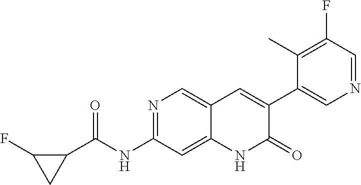 [SMILES: Cc1c(F)cncc1-c1cc2cnc(NC(=O)C3CC3F)cc2[nH]c1=O] |
| 39 | 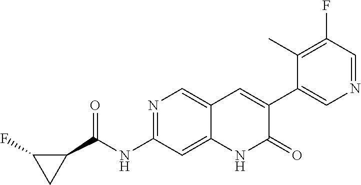 [SMILES: Cc1c(F)cncc1-c1cc2cnc(NC(=O)[C@H]3C[C@@H]3F)cc2[nH]c1=O] | 36 | 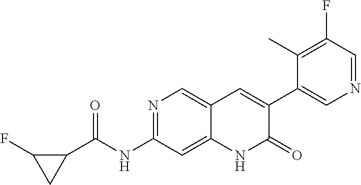 [SMILES: Cc1c(F)cncc1-c1cc2cnc(NC(=O)C3CC3F)cc2[nH]c1=O] |
| 40 | 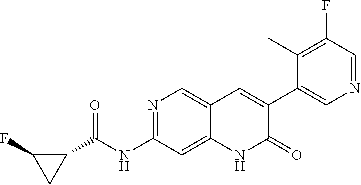 [SMILES: Cc1c(F)cncc1-c1cc2cnc(NC(=O)[C@@H]3C[C@H]3F)cc2[nH]c1=O] | 36 | 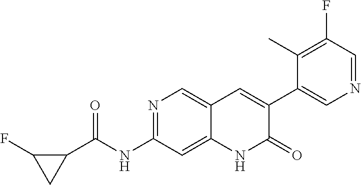 [SMILES: Cc1c(F)cncc1-c1cc2cnc(NC(=O)C3CC3F)cc2[nH]c1=O] |
| 41 | 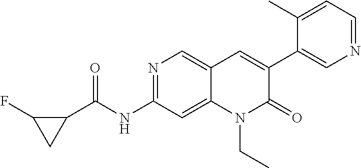 [SMILES: CCn1c(=O)c(-c2cnccc2C)cc2cnc(NC(=O)C3CC3F)cc21] | | |
| 42 | 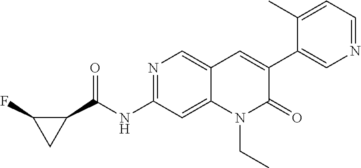 [SMILES: CCn1c(=O)c(-c2cnccc2C)cc2cnc(NC(=O)[C@H]3C[C@H]3F)cc21] | 41 | 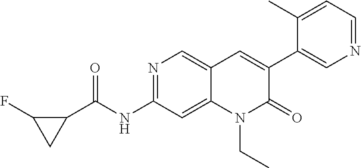 [SMILES: CCn1c(=O)c(-c2cnccc2C)cc2cnc(NC(=O)C3CC3F)cc21] |
| 43 | 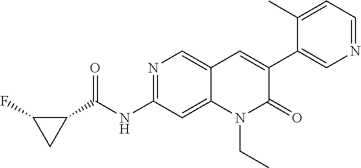 [SMILES: CCn1c(=O)c(-c2cnccc2C)cc2cnc(NC(=O)[C@@H]3C[C@@H]3F)cc21] | 41 | 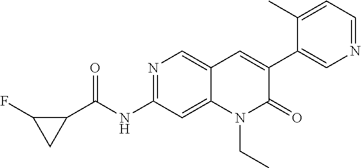 [SMILES: CCn1c(=O)c(-c2cnccc2C)cc2cnc(NC(=O)C3CC3F)cc21] |
| 44 | 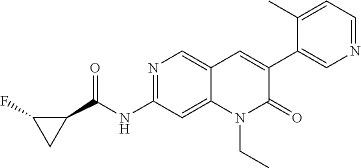 [SMILES: CCn1c(=O)c(-c2cnccc2C)cc2cnc(NC(=O)[C@H]3C[C@@H]3F)cc21] | 41 | 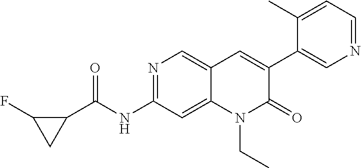 [SMILES: CCn1c(=O)c(-c2cnccc2C)cc2cnc(NC(=O)C3CC3F)cc21] |
| 45 | 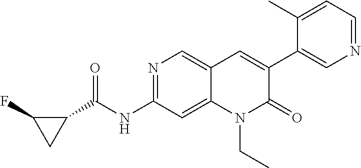 [SMILES: CCn1c(=O)c(-c2cnccc2C)cc2cnc(NC(=O)[C@@H]3C[C@H]3F)cc21] | 41 | 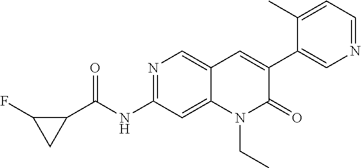 [SMILES: CCn1c(=O)c(-c2cnccc2C)cc2cnc(NC(=O)C3CC3F)cc21] |
| 46 | 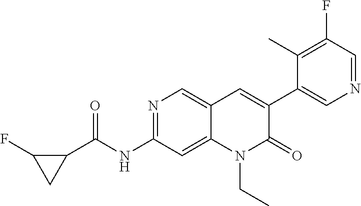 [SMILES: CCn1c(=O)c(-c2cncc(F)c2C)cc2cnc(NC(=O)C3CC3F)cc21] | | |
| 47 | 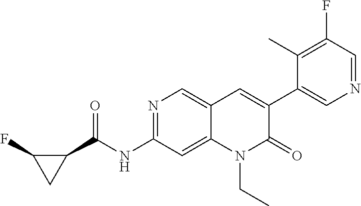 [SMILES: CCn1c(=O)c(-c2cncc(F)c2C)cc2cnc(NC(=O)[C@H]3C[C@H]3F)cc21] | 46 | 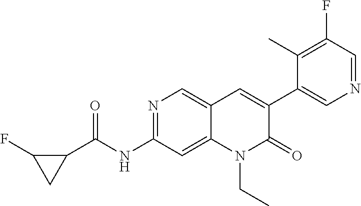 [SMILES: CCn1c(=O)c(-c2cncc(F)c2C)cc2cnc(NC(=O)C3CC3F)cc21] |
| 48 | 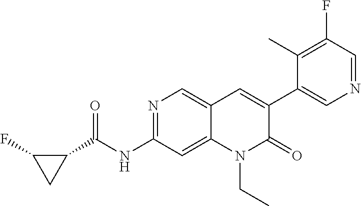 [SMILES: CCn1c(=O)c(-c2cncc(F)c2C)cc2cnc(NC(=O)[C@@H]3C[C@@H]3F)cc21] | 46 | 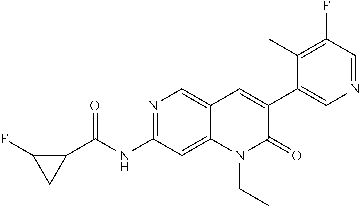 [SMILES: CCn1c(=O)c(-c2cncc(F)c2C)cc2cnc(NC(=O)C3CC3F)cc21] |
| 49 | 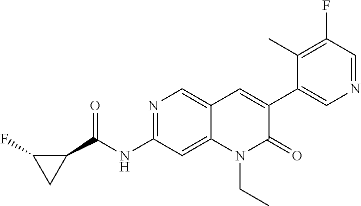 [SMILES: CCn1c(=O)c(-c2cncc(F)c2C)cc2cnc(NC(=O)[C@H]3C[C@@H]3F)cc21] | 46 | 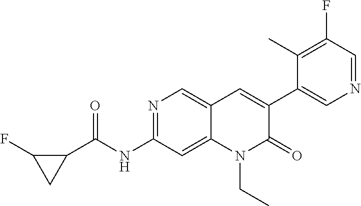 [SMILES: CCn1c(=O)c(-c2cncc(F)c2C)cc2cnc(NC(=O)C3CC3F)cc21] |
| 50 | 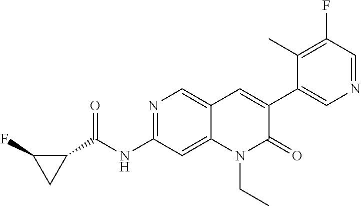 [SMILES: CCn1c(=O)c(-c2cncc(F)c2C)cc2cnc(NC(=O)[C@@H]3C[C@H]3F)cc21] | 46 | 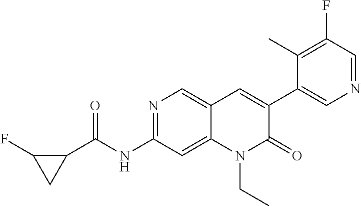 [SMILES: CCn1c(=O)c(-c2cncc(F)c2C)cc2cnc(NC(=O)C3CC3F)cc21] |
| 51 | 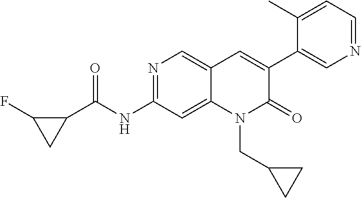 [SMILES: Cc1ccncc1-c1cc2cnc(NC(=O)C3CC3F)cc2n(CC2CC2)c1=O] | | |
| 52 | 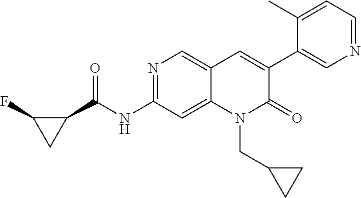 [SMILES: Cc1ccncc1-c1cc2cnc(NC(=O)[C@H]3C[C@H]3F)cc2n(CC2CC2)c1=O] | 51 | 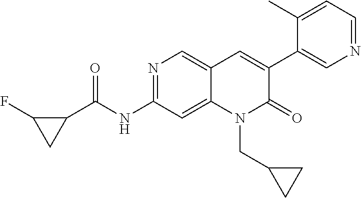 [SMILES: Cc1ccncc1-c1cc2cnc(NC(=O)C3CC3F)cc2n(CC2CC2)c1=O] |
| 53 | 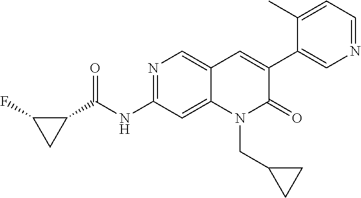 [SMILES: Cc1ccncc1-c1cc2cnc(NC(=O)[C@@H]3C[C@@H]3F)cc2n(CC2CC2)c1=O] | 51 | 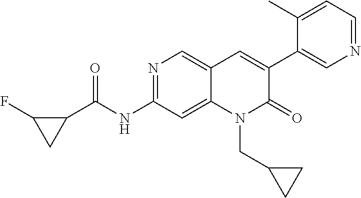 [SMILES: Cc1ccncc1-c1cc2cnc(NC(=O)C3CC3F)cc2n(CC2CC2)c1=O] |
| 54 | 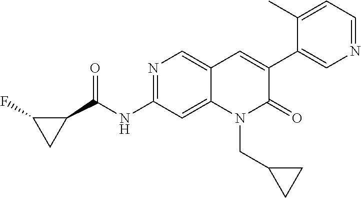 [SMILES: Cc1ccncc1-c1cc2cnc(NC(=O)[C@H]3C[C@@H]3F)cc2n(CC2CC2)c1=O] | 51 | 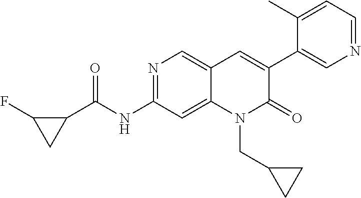 [SMILES: Cc1ccncc1-c1cc2cnc(NC(=O)C3CC3F)cc2n(CC2CC2)c1=O] |
| 55 | 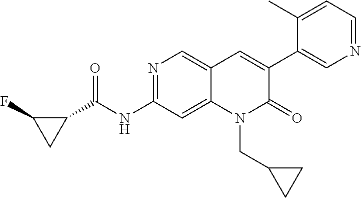 [SMILES: Cc1ccncc1-c1cc2cnc(NC(=O)[C@@H]3C[C@H]3F)cc2n(CC2CC2)c1=O] | 51 | 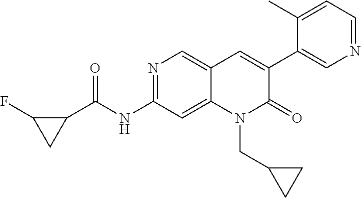 [SMILES: Cc1ccncc1-c1cc2cnc(NC(=O)C3CC3F)cc2n(CC2CC2)c1=O] |
| 56 | 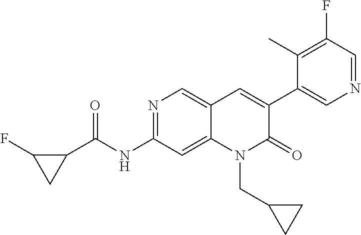 [SMILES: Cc1c(F)cncc1-c1cc2cnc(NC(=O)C3CC3F)cc2n(CC2CC2)c1=O] | | |
| 57 | 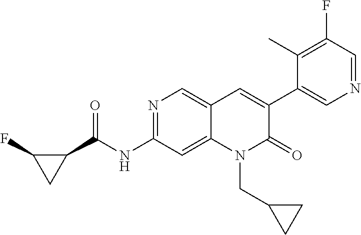 [SMILES: Cc1c(F)cncc1-c1cc2cnc(NC(=O)[C@H]3C[C@H]3F)cc2n(CC2CC2)c1=O] | 56 | 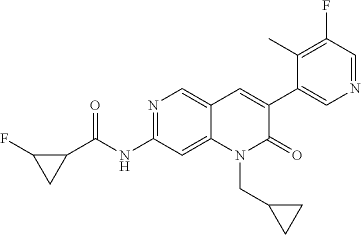 [SMILES: Cc1c(F)cncc1-c1cc2cnc(NC(=O)C3CC3F)cc2n(CC2CC2)c1=O] |
| 58 | 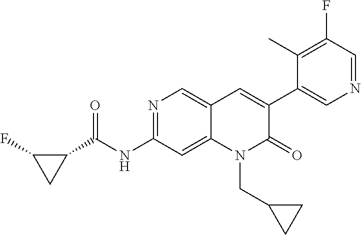 [SMILES: Cc1c(F)cncc1-c1cc2cnc(NC(=O)[C@@H]3C[C@@H]3F)cc2n(CC2CC2)c1=O] | 56 | 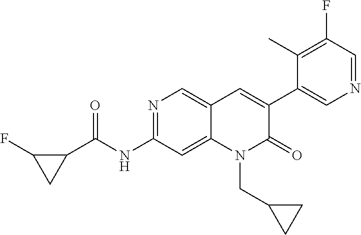 [SMILES: Cc1c(F)cncc1-c1cc2cnc(NC(=O)C3CC3F)cc2n(CC2CC2)c1=O] |
| 59 | 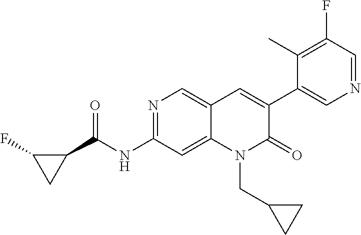 [SMILES: Cc1c(F)cncc1-c1cc2cnc(NC(=O)[C@H]3C[C@@H]3F)cc2n(CC2CC2)c1=O] | 56 | 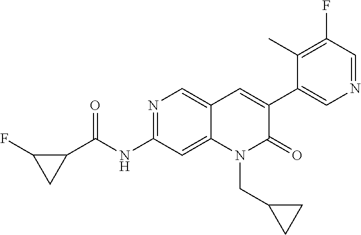 [SMILES: Cc1c(F)cncc1-c1cc2cnc(NC(=O)C3CC3F)cc2n(CC2CC2)c1=O] |
| 60 | 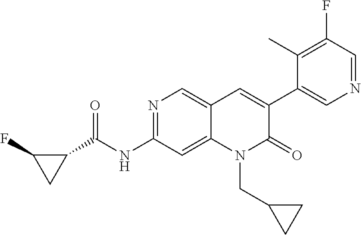 [SMILES: Cc1c(F)cncc1-c1cc2cnc(NC(=O)[C@@H]3C[C@H]3F)cc2n(CC2CC2)c1=O] | 56 | 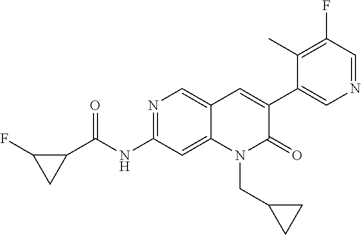 [SMILES: Cc1c(F)cncc1-c1cc2cnc(NC(=O)C3CC3F)cc2n(CC2CC2)c1=O] |
| 61 | 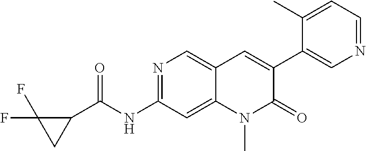 [SMILES: Cc1ccncc1-c1cc2cnc(NC(=O)C3CC3(F)F)cc2n(C)c1=O] | | |
| 62 | 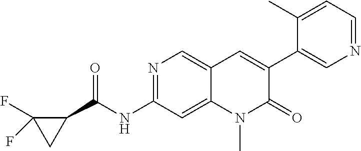 [SMILES: Cc1ccncc1-c1cc2cnc(NC(=O)[C@H]3CC3(F)F)cc2n(C)c1=O] | 61 | 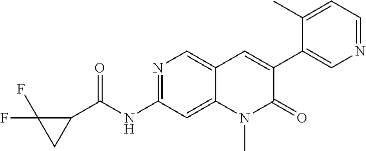 [SMILES: Cc1ccncc1-c1cc2cnc(NC(=O)C3CC3(F)F)cc2n(C)c1=O] |
| 63 | 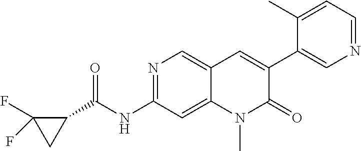 [SMILES: Cc1ccncc1-c1cc2cnc(NC(=O)[C@@H]3CC3(F)F)cc2n(C)c1=O] | 61 | 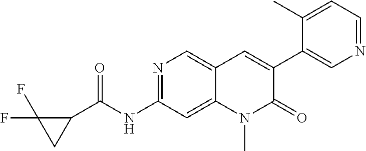 [SMILES: Cc1ccncc1-c1cc2cnc(NC(=O)C3CC3(F)F)cc2n(C)c1=O] |
| 64 | 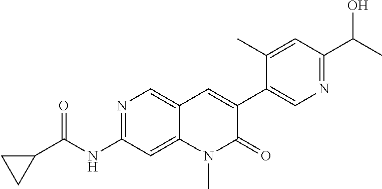 [SMILES: Cc1cc(C(C)O)ncc1-c1cc2cnc(NC(=O)C3CC3)cc2n(C)c1=O] | | |
| 65 | 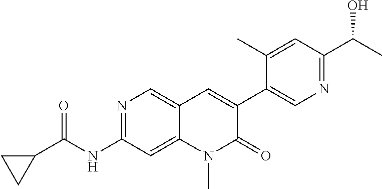 [SMILES: Cc1cc([C@@H](C)O)ncc1-c1cc2cnc(NC(=O)C3CC3)cc2n(C)c1=O] | 64 | 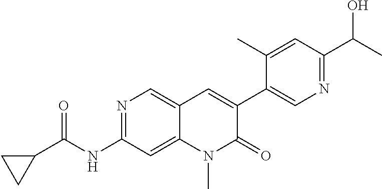 [SMILES: Cc1cc(C(C)O)ncc1-c1cc2cnc(NC(=O)C3CC3)cc2n(C)c1=O] |
| 66 | 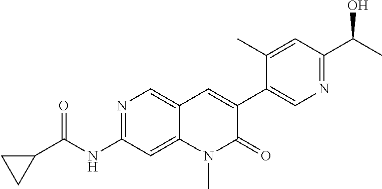 [SMILES: Cc1cc([C@H](C)O)ncc1-c1cc2cnc(NC(=O)C3CC3)cc2n(C)c1=O] | 64 | 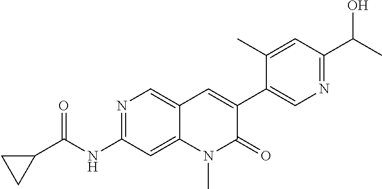 [SMILES: Cc1cc(C(C)O)ncc1-c1cc2cnc(NC(=O)C3CC3)cc2n(C)c1=O] |
| 67 | 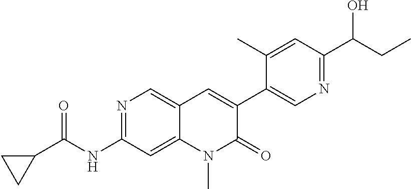 [SMILES: CCC(O)c1cc(C)c(-c2cc3cnc(NC(=O)C4CC4)cc3n(C)c2=O)cn1] | | |
| 68 | 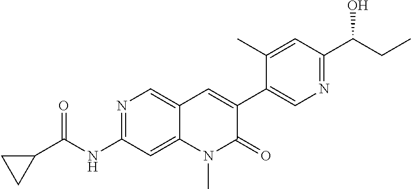 [SMILES: CC[C@@H](O)c1cc(C)c(-c2cc3cnc(NC(=O)C4CC4)cc3n(C)c2=O)cn1] | 67 | 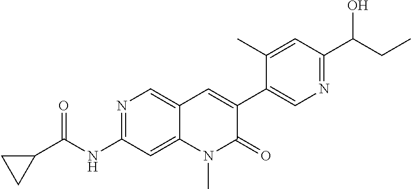 [SMILES: CCC(O)c1cc(C)c(-c2cc3cnc(NC(=O)C4CC4)cc3n(C)c2=O)cn1] |
| 69 | 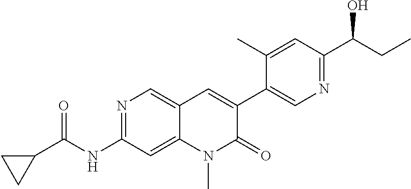 [SMILES: CC[C@H](O)c1cc(C)c(-c2cc3cnc(NC(=O)C4CC4)cc3n(C)c2=O)cn1] | 67 | 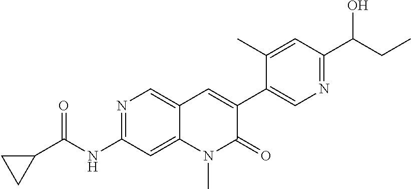 [SMILES: CCC(O)c1cc(C)c(-c2cc3cnc(NC(=O)C4CC4)cc3n(C)c2=O)cn1] |
| 70 | 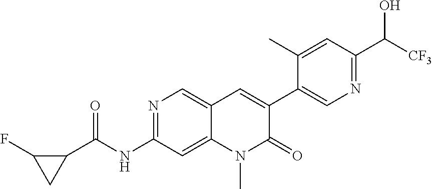 [SMILES: Cc1cc(C(O)C(F)(F)F)ncc1-c1cc2cnc(NC(=O)C3CC3F)cc2n(C)c1=O] | | |
| 71 | 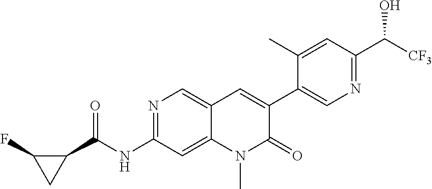 [SMILES: Cc1cc([C@H](O)C(F)(F)F)ncc1-c1cc2cnc(NC(=O)[C@H]3C[C@H]3F)cc2n(C)c1=O] | 70 | 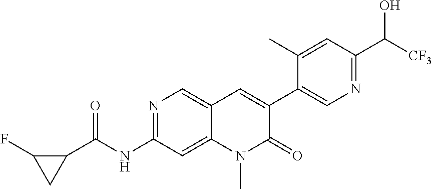 [SMILES: Cc1cc(C(O)C(F)(F)F)ncc1-c1cc2cnc(NC(=O)C3CC3F)cc2n(C)c1=O] |
| 72 | 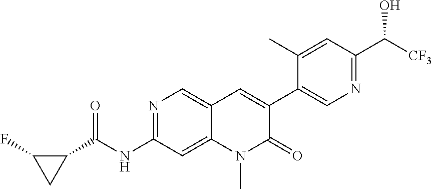 [SMILES: Cc1cc([C@H](O)C(F)(F)F)ncc1-c1cc2cnc(NC(=O)[C@@H]3C[C@@H]3F)cc2n(C)c1=O] | 70 | 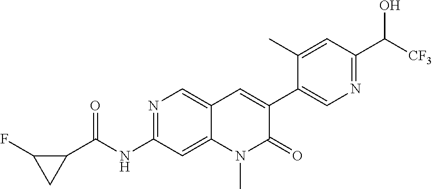 [SMILES: Cc1cc(C(O)C(F)(F)F)ncc1-c1cc2cnc(NC(=O)C3CC3F)cc2n(C)c1=O] |
| 73 | 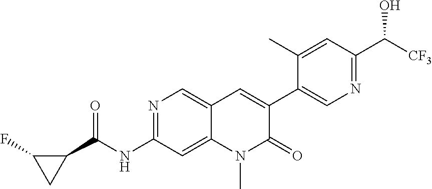 [SMILES: Cc1cc([C@H](O)C(F)(F)F)ncc1-c1cc2cnc(NC(=O)[C@H]3C[C@@H]3F)cc2n(C)c1=O] | 70 | 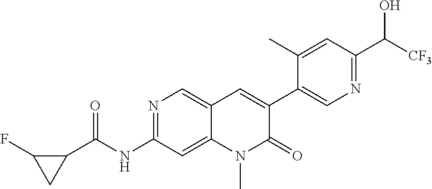 [SMILES: Cc1cc(C(O)C(F)(F)F)ncc1-c1cc2cnc(NC(=O)C3CC3F)cc2n(C)c1=O] |
| 74 | 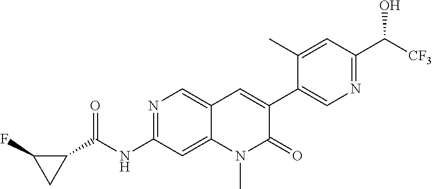 [SMILES: Cc1cc([C@H](O)C(F)(F)F)ncc1-c1cc2cnc(NC(=O)[C@@H]3C[C@H]3F)cc2n(C)c1=O] | 70 | 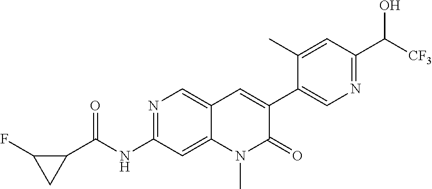 [SMILES: Cc1cc(C(O)C(F)(F)F)ncc1-c1cc2cnc(NC(=O)C3CC3F)cc2n(C)c1=O] |
| 75 | 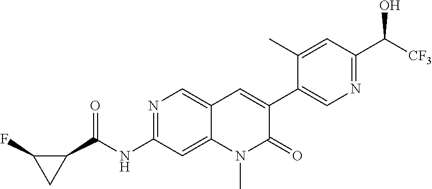 [SMILES: Cc1cc([C@@H](O)C(F)(F)F)ncc1-c1cc2cnc(NC(=O)[C@H]3C[C@H]3F)cc2n(C)c1=O] | 70 | 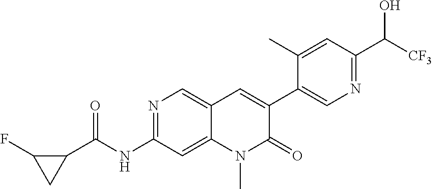 [SMILES: Cc1cc(C(O)C(F)(F)F)ncc1-c1cc2cnc(NC(=O)C3CC3F)cc2n(C)c1=O] |
| 76 | 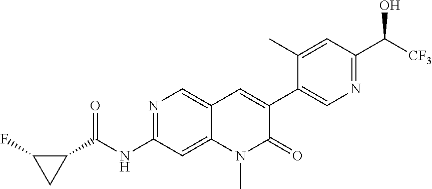 [SMILES: Cc1cc([C@@H](O)C(F)(F)F)ncc1-c1cc2cnc(NC(=O)[C@@H]3C[C@@H]3F)cc2n(C)c1=O] | 70 | 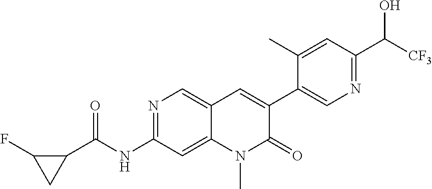 [SMILES: Cc1cc(C(O)C(F)(F)F)ncc1-c1cc2cnc(NC(=O)C3CC3F)cc2n(C)c1=O] |
| 77 | 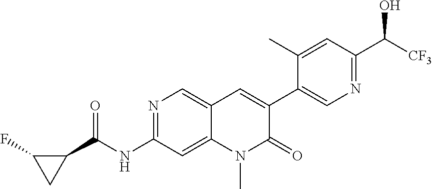 [SMILES: Cc1cc([C@@H](O)C(F)(F)F)ncc1-c1cc2cnc(NC(=O)[C@H]3C[C@@H]3F)cc2n(C)c1=O] | 70 | 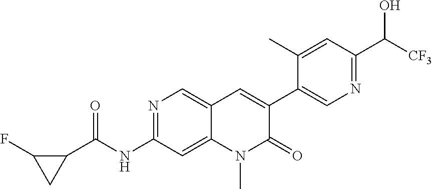 [SMILES: Cc1cc(C(O)C(F)(F)F)ncc1-c1cc2cnc(NC(=O)C3CC3F)cc2n(C)c1=O] |
| 78 | 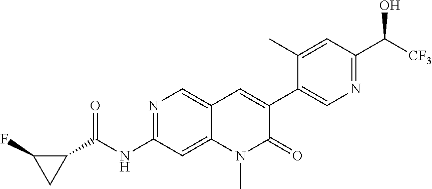 [SMILES: Cc1cc([C@@H](O)C(F)(F)F)ncc1-c1cc2cnc(NC(=O)[C@@H]3C[C@H]3F)cc2n(C)c1=O] | 70 | 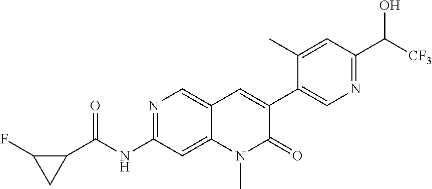 [SMILES: Cc1cc(C(O)C(F)(F)F)ncc1-c1cc2cnc(NC(=O)C3CC3F)cc2n(C)c1=O] |
| 79 | 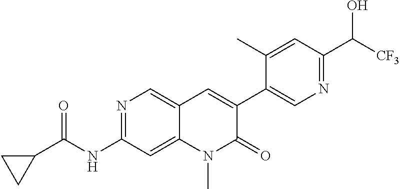 [SMILES: Cc1cc(C(O)C(F)(F)F)ncc1-c1cc2cnc(NC(=O)C3CC3)cc2n(C)c1=O] | | |
| 80 | 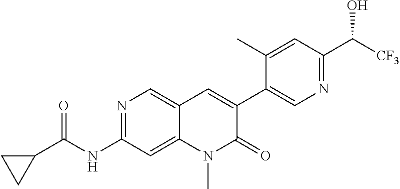 [SMILES: Cc1cc([C@H](O)C(F)(F)F)ncc1-c1cc2cnc(NC(=O)C3CC3)cc2n(C)c1=O] | 79 | 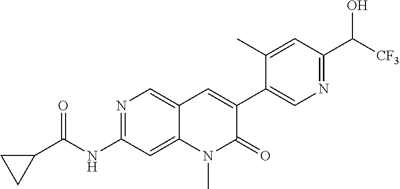 [SMILES: Cc1cc(C(O)C(F)(F)F)ncc1-c1cc2cnc(NC(=O)C3CC3)cc2n(C)c1=O] |
| 81 | 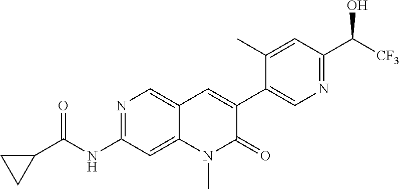 [SMILES: Cc1cc([C@@H](O)C(F)(F)F)ncc1-c1cc2cnc(NC(=O)C3CC3)cc2n(C)c1=O] | 79 | 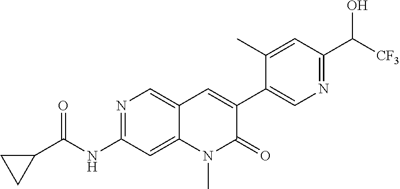 [SMILES: Cc1cc(C(O)C(F)(F)F)ncc1-c1cc2cnc(NC(=O)C3CC3)cc2n(C)c1=O] |
| 82 | 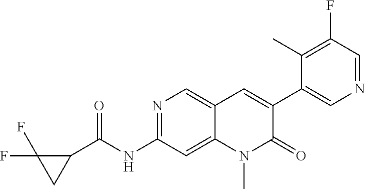 [SMILES: Cc1c(F)cncc1-c1cc2cnc(NC(=O)C3CC3(F)F)cc2n(C)c1=O] | | |
| 83 | 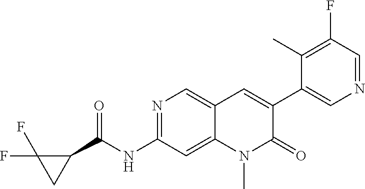 [SMILES: Cc1c(F)cncc1-c1cc2cnc(NC(=O)[C@H]3CC3(F)F)cc2n(C)c1=O] | 82 | 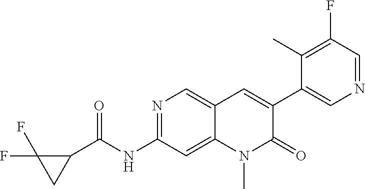 [SMILES: Cc1c(F)cncc1-c1cc2cnc(NC(=O)C3CC3(F)F)cc2n(C)c1=O] |
| 84 | 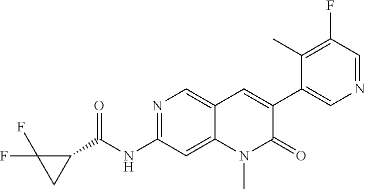 [SMILES: Cc1c(F)cncc1-c1cc2cnc(NC(=O)[C@@H]3CC3(F)F)cc2n(C)c1=O] | 82 | 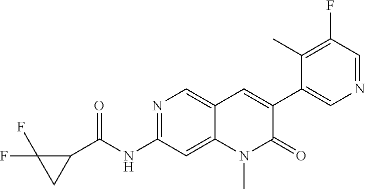 [SMILES: Cc1c(F)cncc1-c1cc2cnc(NC(=O)C3CC3(F)F)cc2n(C)c1=O] |
| 85 | 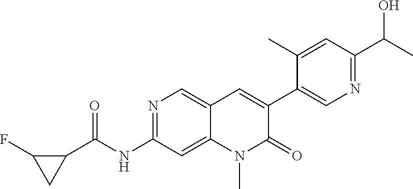 [SMILES: Cc1cc(C(C)O)ncc1-c1cc2cnc(NC(=O)C3CC3F)cc2n(C)c1=O] | | |
| 86 | 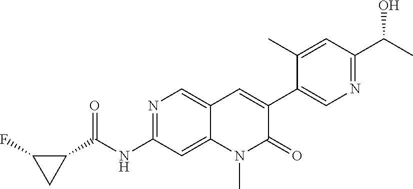 [SMILES: Cc1cc([C@@H](C)O)ncc1-c1cc2cnc(NC(=O)[C@@H]3C[C@@H]3F)cc2n(C)c1=O] | 85 | 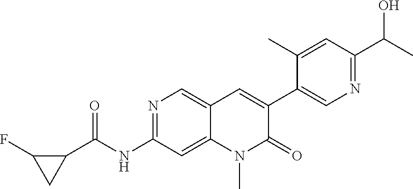 [SMILES: Cc1cc(C(C)O)ncc1-c1cc2cnc(NC(=O)C3CC3F)cc2n(C)c1=O] |
| 87 | 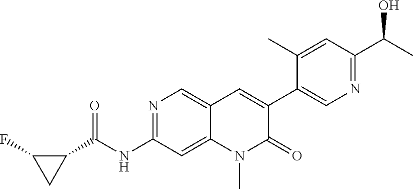 [SMILES: Cc1cc([C@H](C)O)ncc1-c1cc2cnc(NC(=O)[C@@H]3C[C@@H]3F)cc2n(C)c1=O] | 85 | 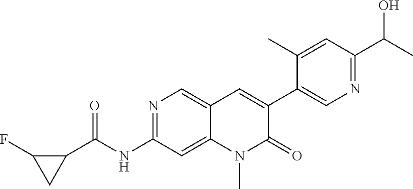 [SMILES: Cc1cc(C(C)O)ncc1-c1cc2cnc(NC(=O)C3CC3F)cc2n(C)c1=O] |
| 88 | 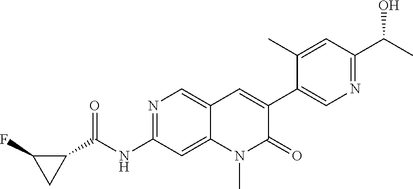 [SMILES: Cc1cc([C@@H](C)O)ncc1-c1cc2cnc(NC(=O)[C@@H]3C[C@H]3F)cc2n(C)c1=O] | 85 | 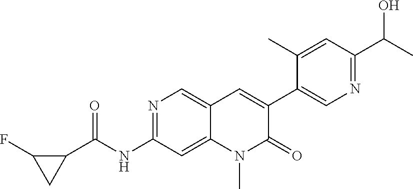 [SMILES: Cc1cc(C(C)O)ncc1-c1cc2cnc(NC(=O)C3CC3F)cc2n(C)c1=O] |
| 89 | 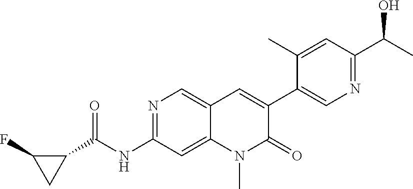 [SMILES: Cc1cc([C@H](C)O)ncc1-c1cc2cnc(NC(=O)[C@@H]3C[C@H]3F)cc2n(C)c1=O] | 85 | 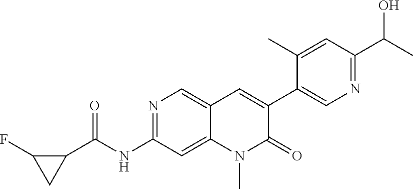 [SMILES: Cc1cc(C(C)O)ncc1-c1cc2cnc(NC(=O)C3CC3F)cc2n(C)c1=O] |
| 90 | 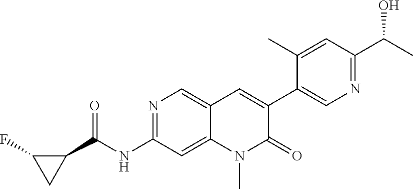 [SMILES: Cc1cc([C@@H](C)O)ncc1-c1cc2cnc(NC(=O)[C@H]3C[C@@H]3F)cc2n(C)c1=O] | 85 | 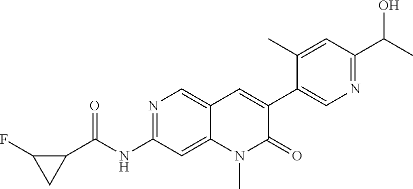 [SMILES: Cc1cc(C(C)O)ncc1-c1cc2cnc(NC(=O)C3CC3F)cc2n(C)c1=O] |
| 91 | 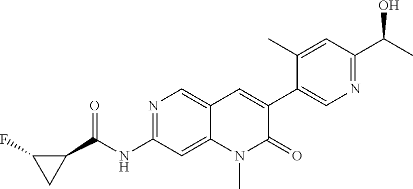 [SMILES: Cc1cc([C@H](C)O)ncc1-c1cc2cnc(NC(=O)[C@H]3C[C@@H]3F)cc2n(C)c1=O] | 85 | 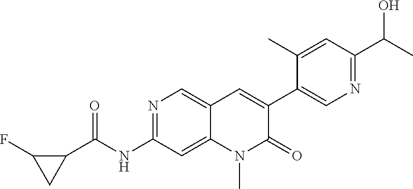 [SMILES: Cc1cc(C(C)O)ncc1-c1cc2cnc(NC(=O)C3CC3F)cc2n(C)c1=O] |
| 92 | 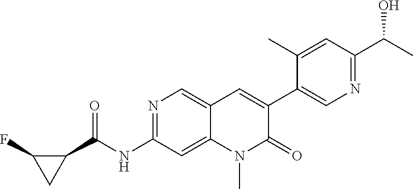 [SMILES: Cc1cc([C@@H](C)O)ncc1-c1cc2cnc(NC(=O)[C@H]3C[C@H]3F)cc2n(C)c1=O] | 85 | 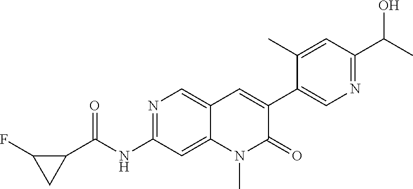 [SMILES: Cc1cc(C(C)O)ncc1-c1cc2cnc(NC(=O)C3CC3F)cc2n(C)c1=O] |
| 93 | 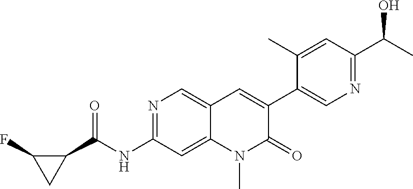 [SMILES: Cc1cc([C@H](C)O)ncc1-c1cc2cnc(NC(=O)[C@H]3C[C@H]3F)cc2n(C)c1=O] | 85 | 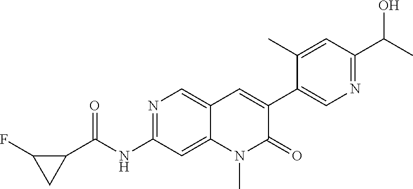 [SMILES: Cc1cc(C(C)O)ncc1-c1cc2cnc(NC(=O)C3CC3F)cc2n(C)c1=O] |
| 94 | 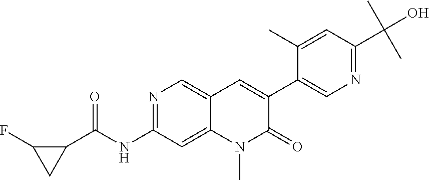 [SMILES: Cc1cc(C(C)(C)O)ncc1-c1cc2cnc(NC(=O)C3CC3F)cc2n(C)c1=O] | | |
| 95 | 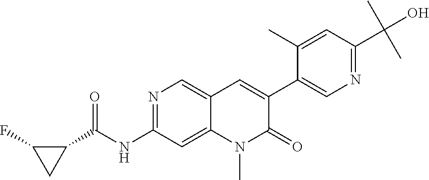 [SMILES: Cc1cc(C(C)(C)O)ncc1-c1cc2cnc(NC(=O)[C@@H]3C[C@@H]3F)cc2n(C)c1=O] | 94 | 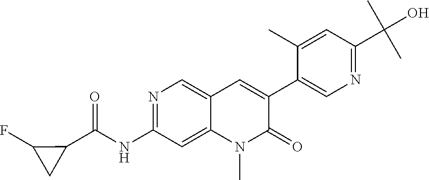 [SMILES: Cc1cc(C(C)(C)O)ncc1-c1cc2cnc(NC(=O)C3CC3F)cc2n(C)c1=O] |
| 96 | 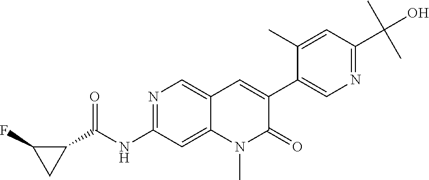 [SMILES: Cc1cc(C(C)(C)O)ncc1-c1cc2cnc(NC(=O)[C@@H]3C[C@H]3F)cc2n(C)c1=O] | 94 | 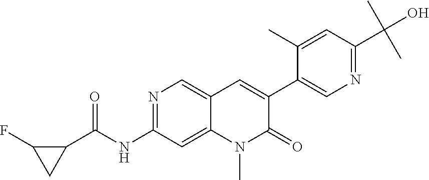 [SMILES: Cc1cc(C(C)(C)O)ncc1-c1cc2cnc(NC(=O)C3CC3F)cc2n(C)c1=O] |
| 97 | 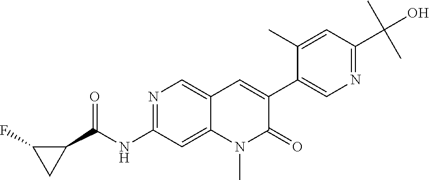 [SMILES: Cc1cc(C(C)(C)O)ncc1-c1cc2cnc(NC(=O)[C@H]3C[C@@H]3F)cc2n(C)c1=O] | 94 | 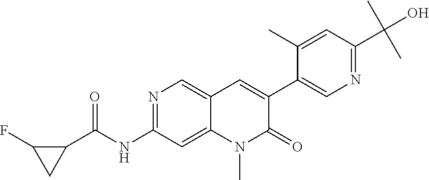 [SMILES: Cc1cc(C(C)(C)O)ncc1-c1cc2cnc(NC(=O)C3CC3F)cc2n(C)c1=O] |
| 98 | 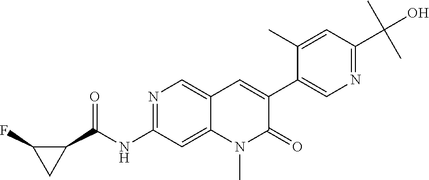 [SMILES: Cc1cc(C(C)(C)O)ncc1-c1cc2cnc(NC(=O)[C@H]3C[C@H]3F)cc2n(C)c1=O] | 94 | 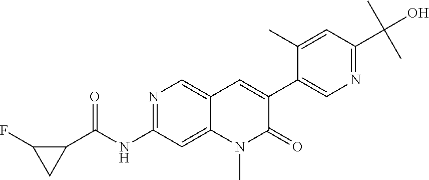 [SMILES: Cc1cc(C(C)(C)O)ncc1-c1cc2cnc(NC(=O)C3CC3F)cc2n(C)c1=O] |
| 99 | 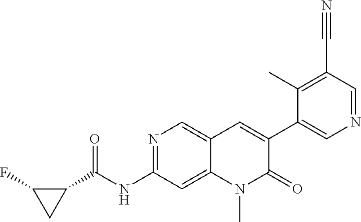 [SMILES: Cc1c(C#N)cncc1-c1cc2cnc(NC(=O)[C@@H]3C[C@@H]3F)cc2n(C)c1=O] | 99-1 | 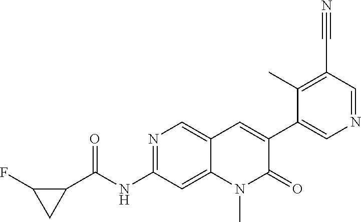 [SMILES: Cc1c(C#N)cncc1-c1cc2cnc(NC(=O)C3CC3F)cc2n(C)c1=O] |
| 100 | 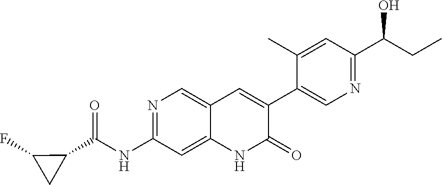 [SMILES: CC[C@H](O)c1cc(C)c(-c2cc3cnc(NC(=O)[C@@H]4C[C@@H]4F)cc3[nH]c2=O)cn1] | 100-1 | 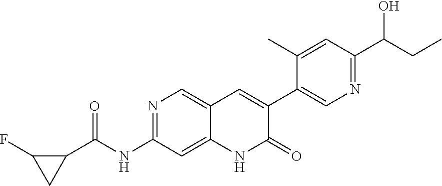 [SMILES: CCC(O)c1cc(C)c(-c2cc3cnc(NC(=O)C4CC4F)cc3[nH]c2=O)cn1] |
| 101 | 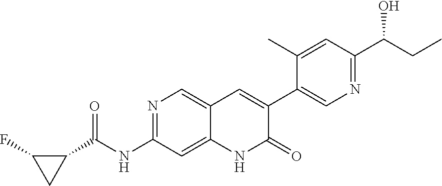 [SMILES: CC[C@@H](O)c1cc(C)c(-c2cc3cnc(NC(=O)[C@@H]4C[C@@H]4F)cc3[nH]c2=O)cn1] | 100-1 | 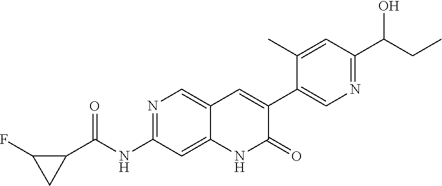 [SMILES: CCC(O)c1cc(C)c(-c2cc3cnc(NC(=O)C4CC4F)cc3[nH]c2=O)cn1] |
| 102 | 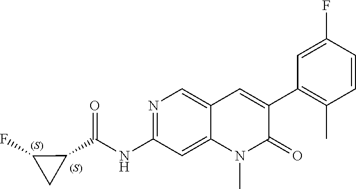 | 102-1 | 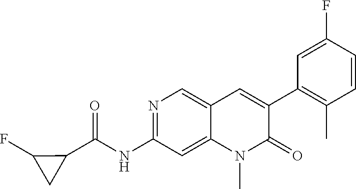 [SMILES: Cc1ccc(F)cc1-c1cc2cnc(NC(=O)C3CC3F)cc2n(C)c1=O] |
| 103 | 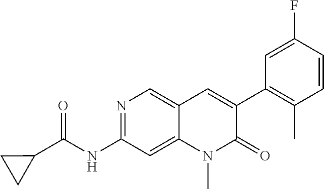 [SMILES: Cc1ccc(F)cc1-c1cc2cnc(NC(=O)C3CC3)cc2n(C)c1=O] | | |
| 104 | 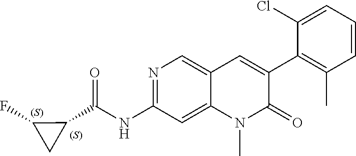 | 104-1 | 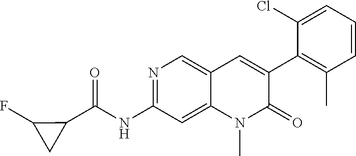 [SMILES: Cc1cccc(Cl)c1-c1cc2cnc(NC(=O)C3CC3F)cc2n(C)c1=O] |
| 105 | 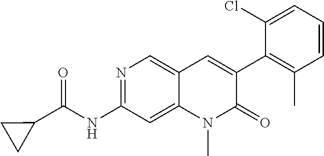 [SMILES: Cc1cccc(Cl)c1-c1cc2cnc(NC(=O)C3CC3)cc2n(C)c1=O] | | |
| 106 | 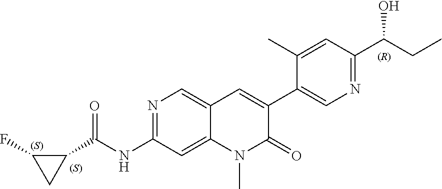 | 106-1 | 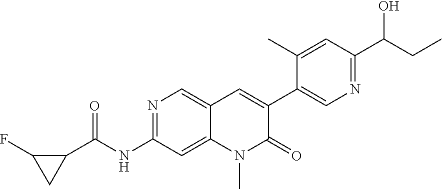 [SMILES: CCC(O)c1cc(C)c(-c2cc3cnc(NC(=O)C4CC4F)cc3n(C)c2=O)cn1] |
| 107 | 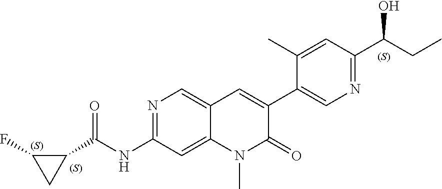 | 106-1 | 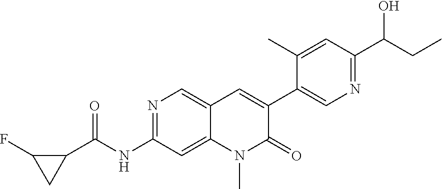 [SMILES: CCC(O)c1cc(C)c(-c2cc3cnc(NC(=O)C4CC4F)cc3n(C)c2=O)cn1] |
| 108 | 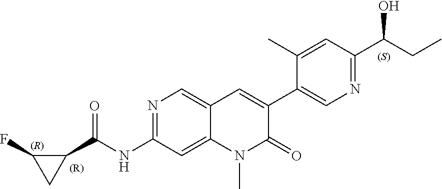 | 106-1 | 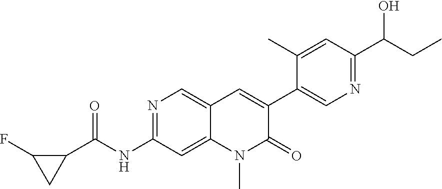 [SMILES: CCC(O)c1cc(C)c(-c2cc3cnc(NC(=O)C4CC4F)cc3n(C)c2=O)cn1] |
| 109 | 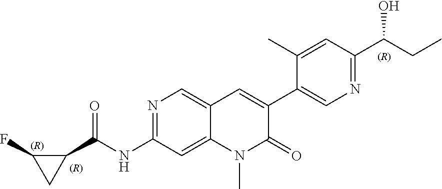 | 106-1 | 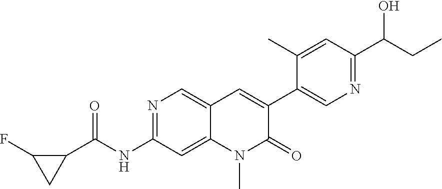 [SMILES: CCC(O)c1cc(C)c(-c2cc3cnc(NC(=O)C4CC4F)cc3n(C)c2=O)cn1] |
| 110 | 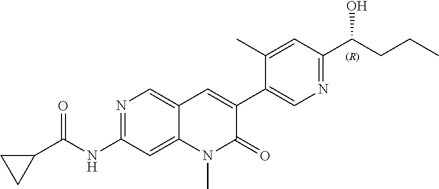 | 110-1 | 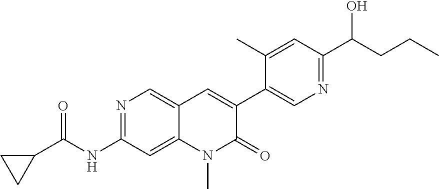 [SMILES: CCCC(O)c1cc(C)c(-c2cc3cnc(NC(=O)C4CC4)cc3n(C)c2=O)cn1] |
| 111 | 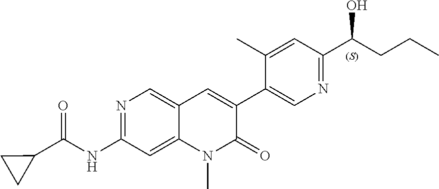 | 110-1 | 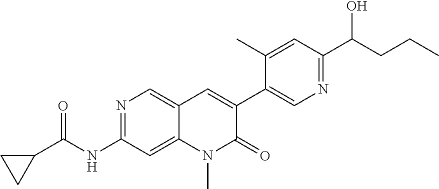 [SMILES: CCCC(O)c1cc(C)c(-c2cc3cnc(NC(=O)C4CC4)cc3n(C)c2=O)cn1] |
| 112 | 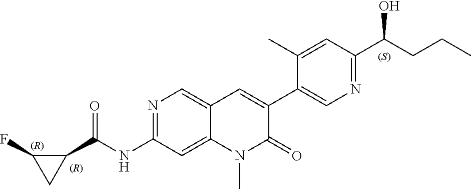 | 112-1 | 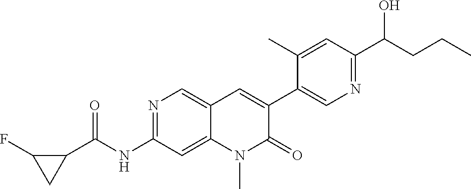 [SMILES: CCCC(O)c1cc(C)c(-c2cc3cnc(NC(=O)C4CC4F)cc3n(C)c2=O)cn1] |
| 113 | 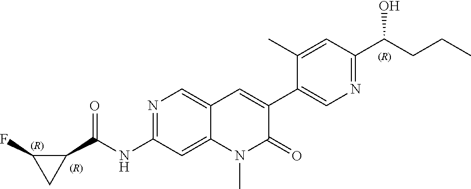 | 112-1 | 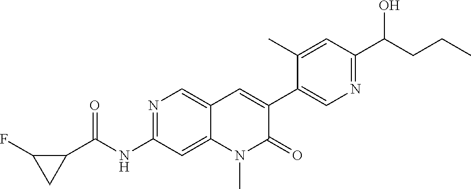 [SMILES: CCCC(O)c1cc(C)c(-c2cc3cnc(NC(=O)C4CC4F)cc3n(C)c2=O)cn1] |
| 114 | 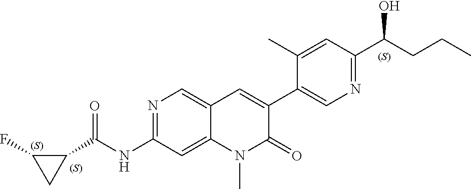 | 112-1 | 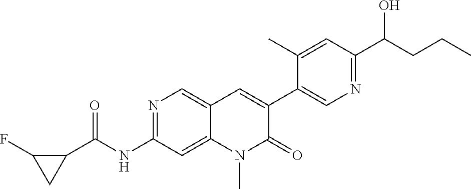 [SMILES: CCCC(O)c1cc(C)c(-c2cc3cnc(NC(=O)C4CC4F)cc3n(C)c2=O)cn1] |
| 115 | 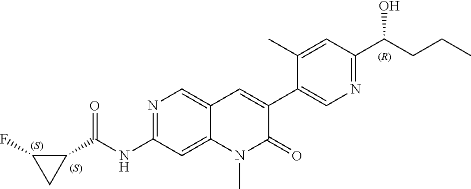 | 112-1 | 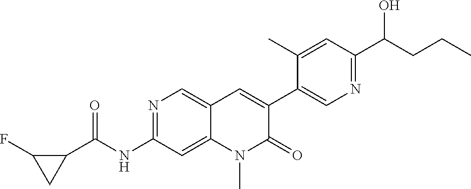 [SMILES: CCCC(O)c1cc(C)c(-c2cc3cnc(NC(=O)C4CC4F)cc3n(C)c2=O)cn1] |
| 116 | 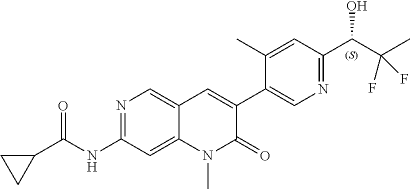 | 116-1 | 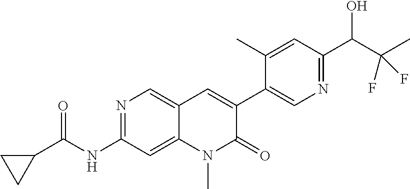 [SMILES: Cc1cc(C(O)C(C)(F)F)ncc1-c1cc2cnc(NC(=O)C3CC3)cc2n(C)c1=O] |
| 117 | 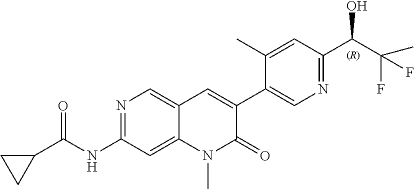 | 116-1 | 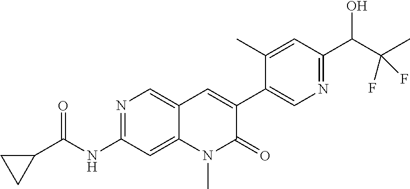 [SMILES: Cc1cc(C(O)C(C)(F)F)ncc1-c1cc2cnc(NC(=O)C3CC3)cc2n(C)c1=O] |
| 118 | 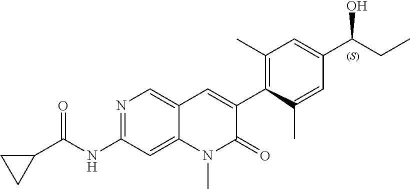 | 118-1 | 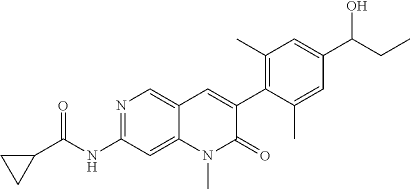 [SMILES: CCC(O)c1cc(C)c(-c2cc3cnc(NC(=O)C4CC4)cc3n(C)c2=O)c(C)c1] |
| 119 | 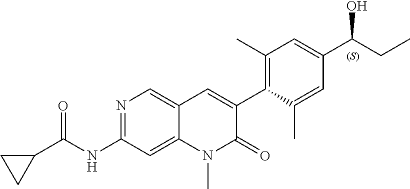 | 118-1 | 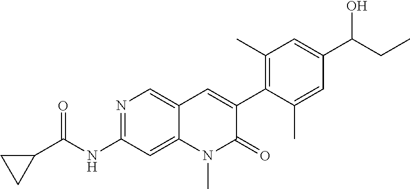 [SMILES: CCC(O)c1cc(C)c(-c2cc3cnc(NC(=O)C4CC4)cc3n(C)c2=O)c(C)c1] |
| 120 | 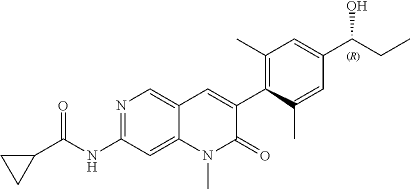 | 118-1 | 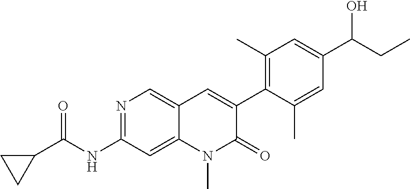 [SMILES: CCC(O)c1cc(C)c(-c2cc3cnc(NC(=O)C4CC4)cc3n(C)c2=O)c(C)c1] |
| 121 | 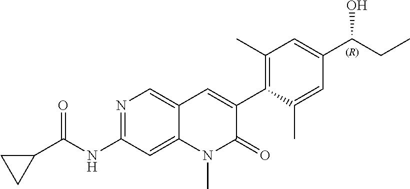 | 118-1 | 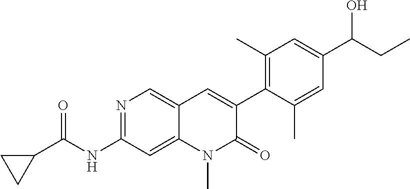 [SMILES: CCC(O)c1cc(C)c(-c2cc3cnc(NC(=O)C4CC4)cc3n(C)c2=O)c(C)c1] |
| 122 | 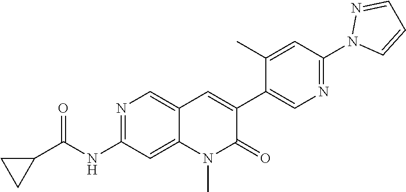 [SMILES: Cc1cc(-n2cccn2)ncc1-c1cc2cnc(NC(=O)C3CC3)cc2n(C)c1=O] | | |
| 123 | 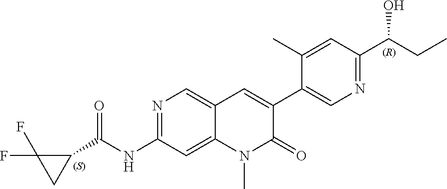 | 123-1 | 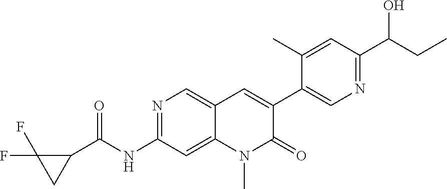 [SMILES: CCC(O)c1cc(C)c(-c2cc3cnc(NC(=O)C4CC4(F)F)cc3n(C)c2=O)cn1] |
| 124 | 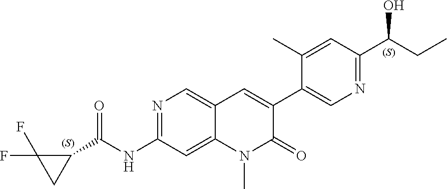 | 123-1 | 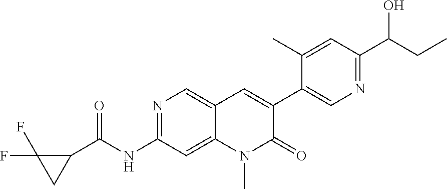 [SMILES: CCC(O)c1cc(C)c(-c2cc3cnc(NC(=O)C4CC4(F)F)cc3n(C)c2=O)cn1] |
| 125 | 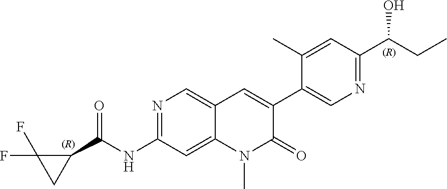 | 123-1 | 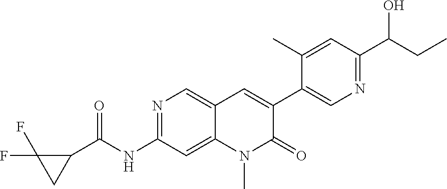 [SMILES: CCC(O)c1cc(C)c(-c2cc3cnc(NC(=O)C4CC4(F)F)cc3n(C)c2=O)cn1] |
| 126 | 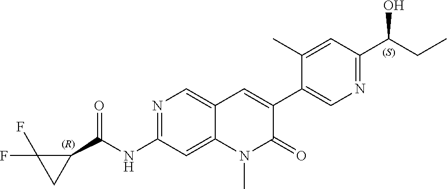 | 123-1 | 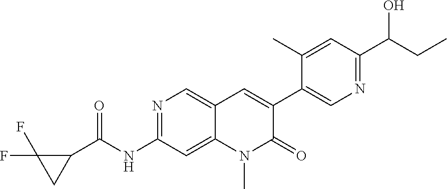 [SMILES: CCC(O)c1cc(C)c(-c2cc3cnc(NC(=O)C4CC4(F)F)cc3n(C)c2=O)cn1] |
| 127 | 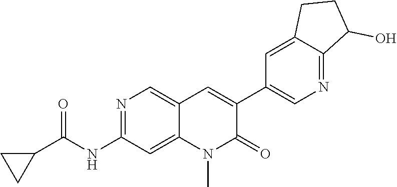 [SMILES: Cn1c(=O)c(-c2cnc3c(c2)CCC3O)cc2cnc(NC(=O)C3CC3)cc21] | | |
| 128 | 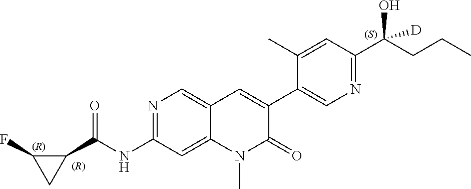 | 128-1 | 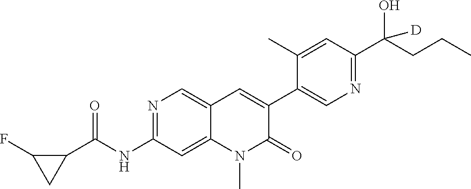 [SMILES: [2H]C(O)(CCC)c1cc(C)c(-c2cc3cnc(NC(=O)C4CC4F)cc3n(C)c2=O)cn1] |
| 129 | 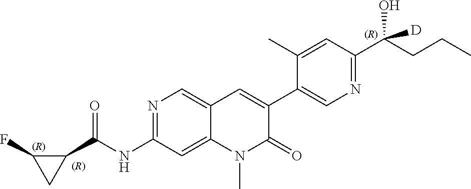 | 128-1 | 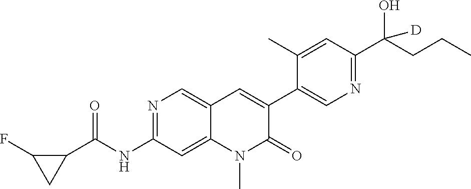 [SMILES: [2H]C(O)(CCC)c1cc(C)c(-c2cc3cnc(NC(=O)C4CC4F)cc3n(C)c2=O)cn1] |
| 130 | 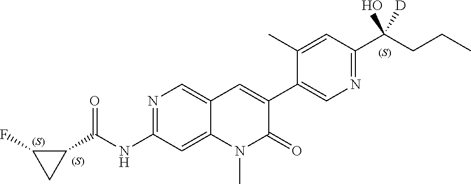 | 128-1 | 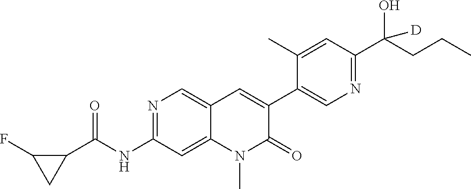 [SMILES: [2H]C(O)(CCC)c1cc(C)c(-c2cc3cnc(NC(=O)C4CC4F)cc3n(C)c2=O)cn1] |
| 131 | 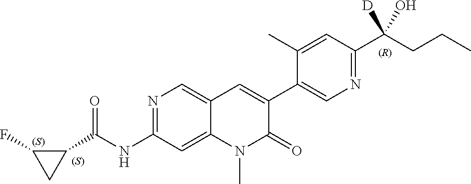 | 128-1 | 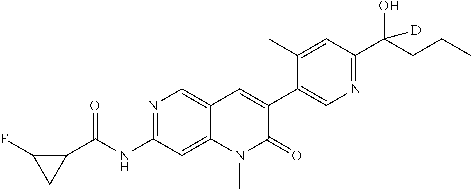 [SMILES: [2H]C(O)(CCC)c1cc(C)c(-c2cc3cnc(NC(=O)C4CC4F)cc3n(C)c2=O)cn1] |
| 132 | 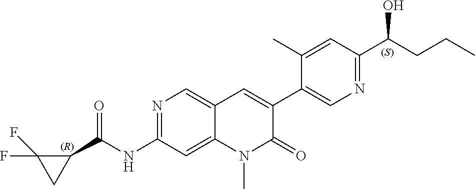 | 132-1 | 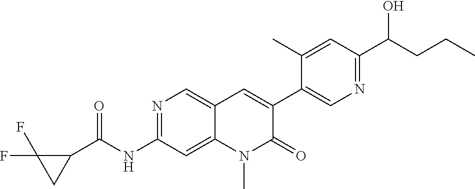 [SMILES: CCCC(O)c1cc(C)c(-c2cc3cnc(NC(=O)C4CC4(F)F)cc3n(C)c2=O)cn1] |
| 133 | 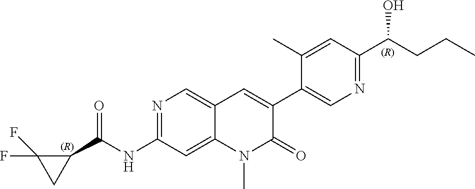 | 132-1 | 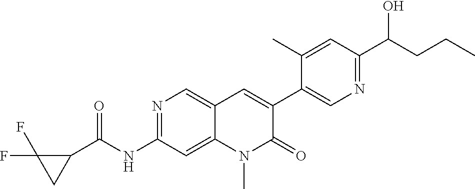 [SMILES: CCCC(O)c1cc(C)c(-c2cc3cnc(NC(=O)C4CC4(F)F)cc3n(C)c2=O)cn1] |
| 134 | 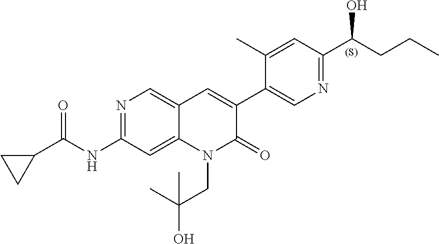 | 134-1 | 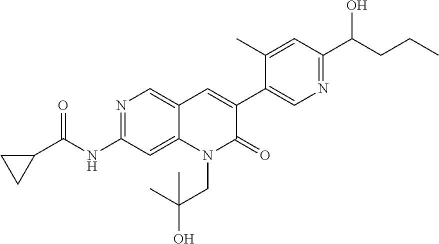 [SMILES: CCCC(O)c1cc(C)c(-c2cc3cnc(NC(=O)C4CC4)cc3n(CC(C)(C)O)c2=O)cn1] |
| 135 | 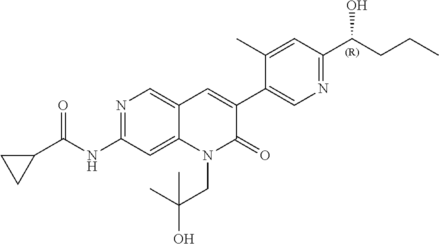 | 134-1 | 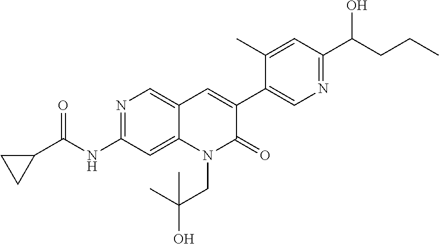 [SMILES: CCCC(O)c1cc(C)c(-c2cc3cnc(NC(=O)C4CC4)cc3n(CC(C)(C)O)c2=O)cn1] |
| 136 | 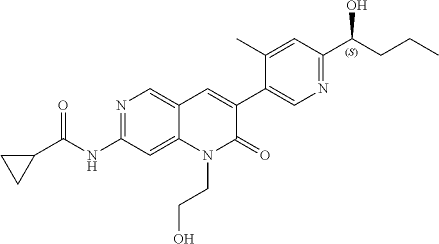 | 136-1 | 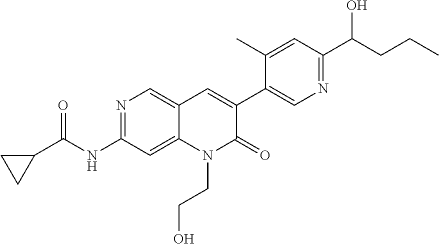 [SMILES: CCCC(O)c1cc(C)c(-c2cc3cnc(NC(=O)C4CC4)cc3n(CCO)c2=O)cn1] |
| 137 | 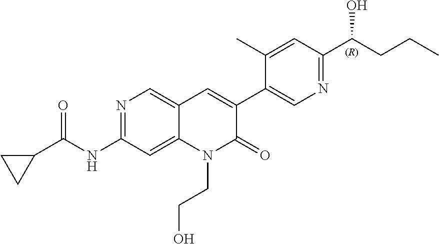 | 136-1 | 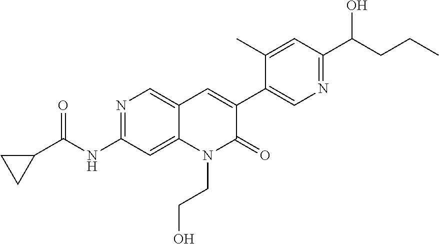 [SMILES: CCCC(O)c1cc(C)c(-c2cc3cnc(NC(=O)C4CC4)cc3n(CCO)c2=O)cn1] |
| 138 | 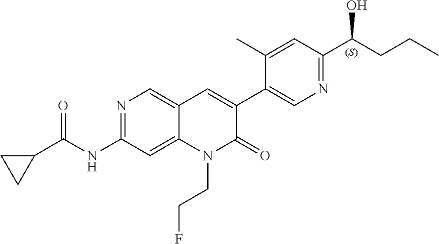 | 138-1 | 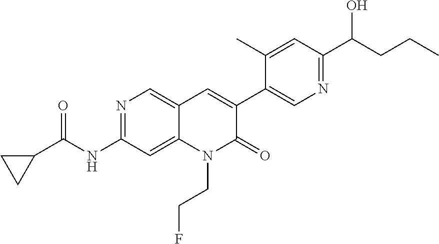 [SMILES: CCCC(O)c1cc(C)c(-c2cc3cnc(NC(=O)C4CC4)cc3n(CCF)c2=O)cn1] |
| 139 | 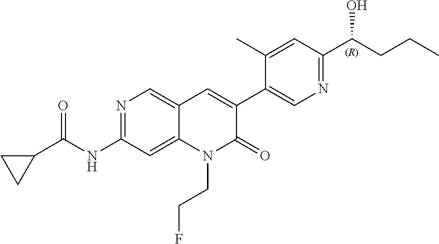 | 138-1 | 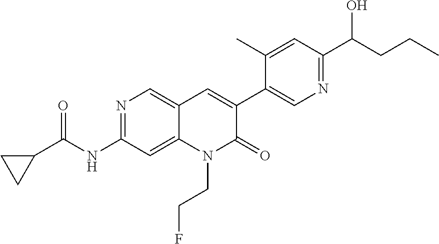 [SMILES: CCCC(O)c1cc(C)c(-c2cc3cnc(NC(=O)C4CC4)cc3n(CCF)c2=O)cn1] |
| 140 | 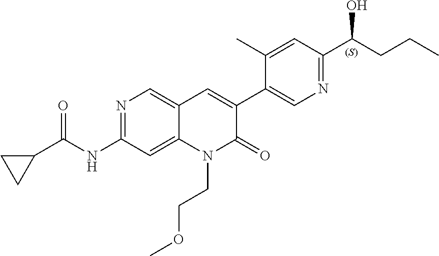 | 140-1 | 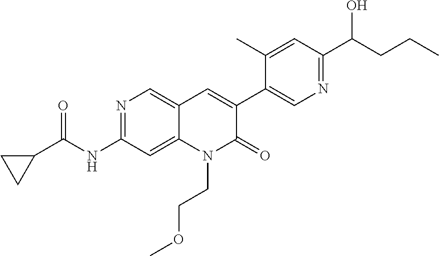 [SMILES: CCCC(O)c1cc(C)c(-c2cc3cnc(NC(=O)C4CC4)cc3n(CCOC)c2=O)cn1] |
| 141 | 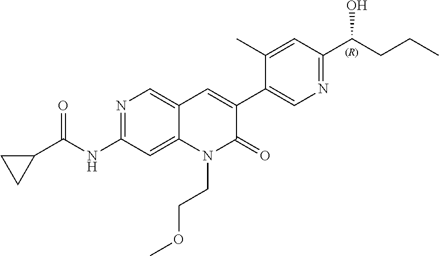 | 140-1 | 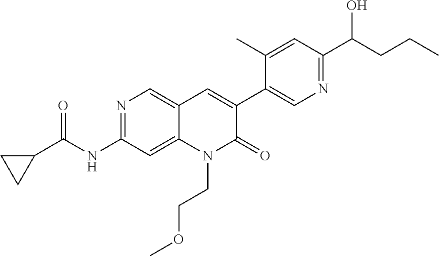 [SMILES: CCCC(O)c1cc(C)c(-c2cc3cnc(NC(=O)C4CC4)cc3n(CCOC)c2=O)cn1] |
| 142 | 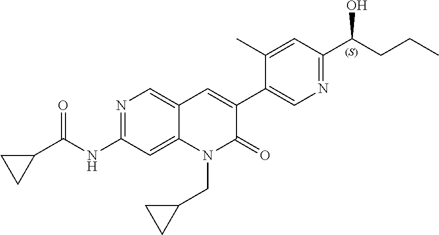 | 142-1 | 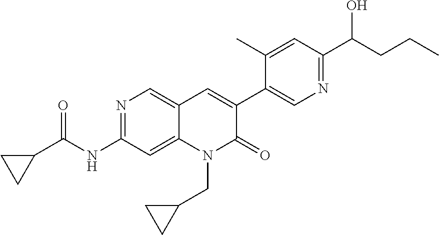 [SMILES: CCCC(O)c1cc(C)c(-c2cc3cnc(NC(=O)C4CC4)cc3n(CC3CC3)c2=O)cn1] |
| 143 | 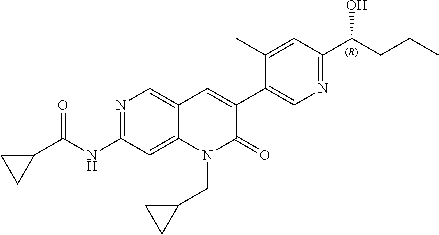 | 142-1 | 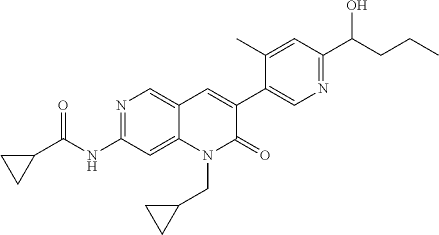 [SMILES: CCCC(O)c1cc(C)c(-c2cc3cnc(NC(=O)C4CC4)cc3n(CC3CC3)c2=O)cn1] |
| 144 | 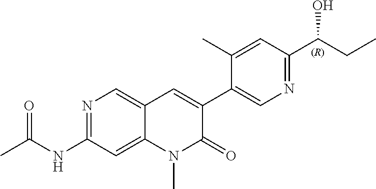 | 144-1 | 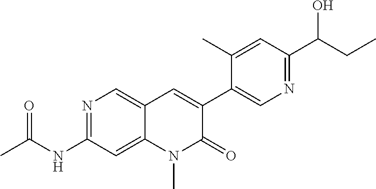 [SMILES: CCC(O)c1cc(C)c(-c2cc3cnc(NC(C)=O)cc3n(C)c2=O)cn1] |
| 145 | 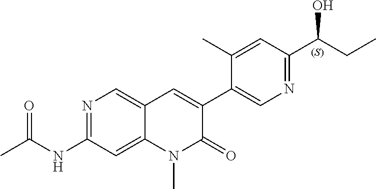 | 144-1 | 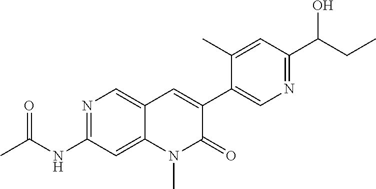 [SMILES: CCC(O)c1cc(C)c(-c2cc3cnc(NC(C)=O)cc3n(C)c2=O)cn1] |
| 146 | 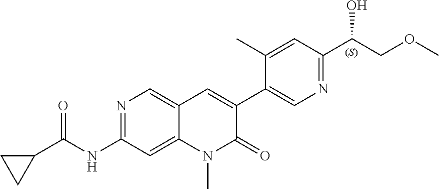 | 146-1 | 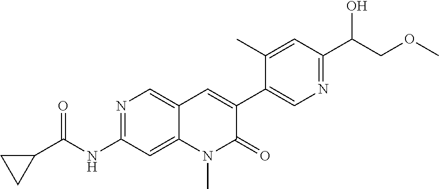 [SMILES: COCC(O)c1cc(C)c(-c2cc3cnc(NC(=O)C4CC4)cc3n(C)c2=O)cn1] |
| 147 | 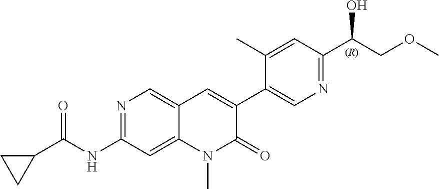 | 146-1 | 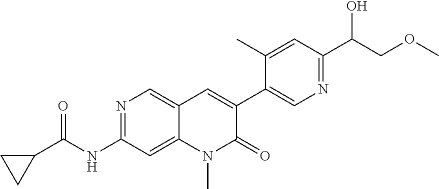 [SMILES: COCC(O)c1cc(C)c(-c2cc3cnc(NC(=O)C4CC4)cc3n(C)c2=O)cn1] |
| 148 | 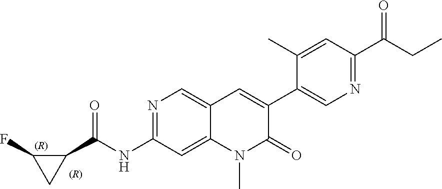 | 148-1 | 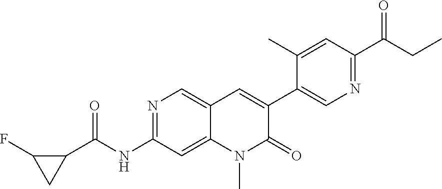 [SMILES: CCC(=O)c1cc(C)c(-c2cc3cnc(NC(=O)C4CC4F)cc3n(C)c2=O)cn1] |
| 149 | 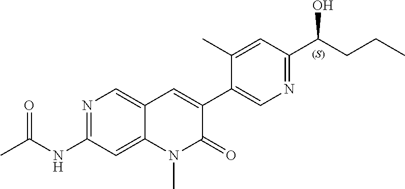 | 149-1 | 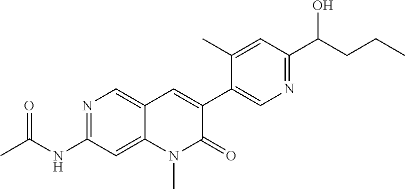 [SMILES: CCCC(O)c1cc(C)c(-c2cc3cnc(NC(C)=O)cc3n(C)c2=O)cn1] |
| 150 | 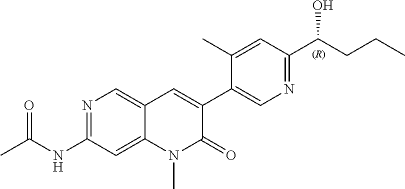 | 149-1 | 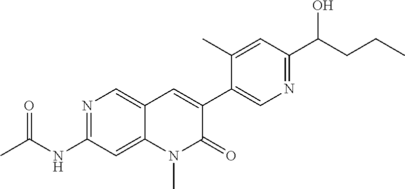 [SMILES: CCCC(O)c1cc(C)c(-c2cc3cnc(NC(C)=O)cc3n(C)c2=O)cn1] |
| 151 | 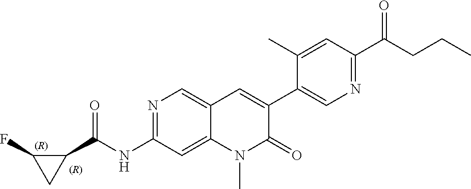 | 151-1 | 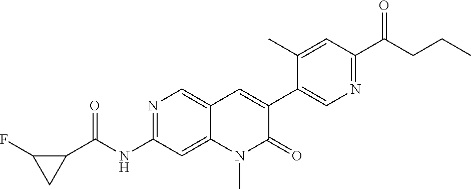 [SMILES: CCCC(=O)c1cc(C)c(-c2cc3cnc(NC(=O)C4CC4F)cc3n(C)c2=O)cn1] |
| 152 | 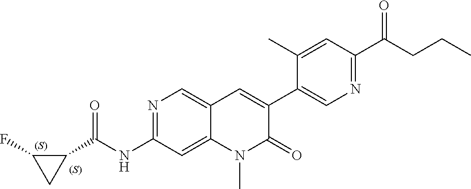 | 151-1 | 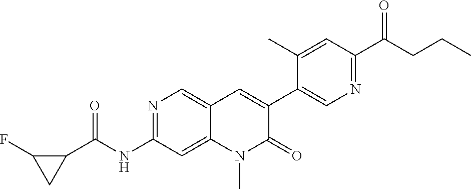 [SMILES: CCCC(=O)c1cc(C)c(-c2cc3cnc(NC(=O)C4CC4F)cc3n(C)c2=O)cn1] |
| 153 | 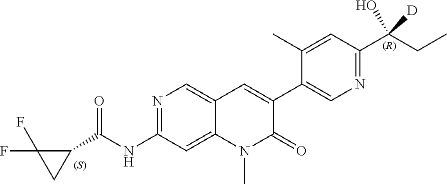 | 153-1 | 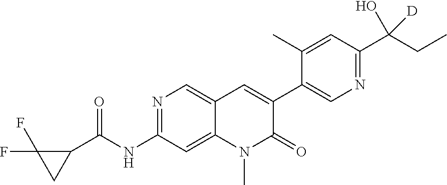 [SMILES: [2H]C(O)(CC)c1cc(C)c(-c2cc3cnc(NC(=O)C4CC4(F)F)cc3n(C)c2=O)cn1] |
| 154 | 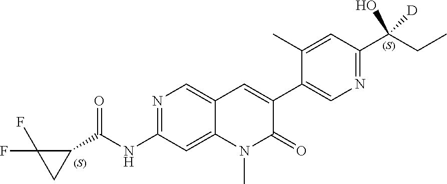 | 153-1 | 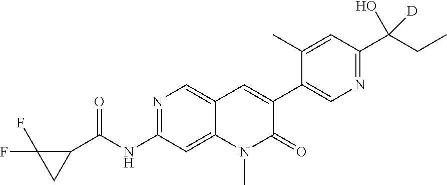 [SMILES: [2H]C(O)(CC)c1cc(C)c(-c2cc3cnc(NC(=O)C4CC4(F)F)cc3n(C)c2=O)cn1] |
| 155 | 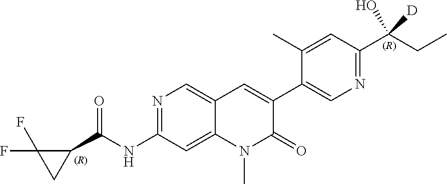 | 153-1 | 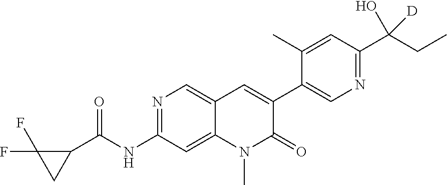 [SMILES: [2H]C(O)(CC)c1cc(C)c(-c2cc3cnc(NC(=O)C4CC4(F)F)cc3n(C)c2=O)cn1] |
| 156 | 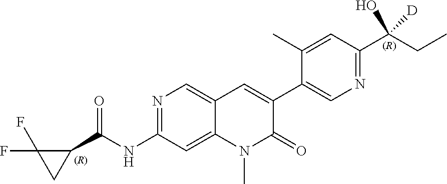 | 153-1 | 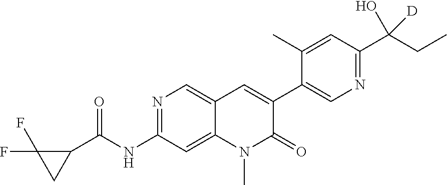 [SMILES: [2H]C(O)(CC)c1cc(C)c(-c2cc3cnc(NC(=O)C4CC4(F)F)cc3n(C)c2=O)cn1] |
| 157 | 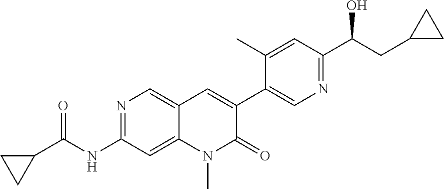 [SMILES: Cc1cc([C@@H](O)CC2CC2)ncc1-c1cc2cnc(NC(=O)C3CC3)cc2n(C)c1=O] | 157-1 | 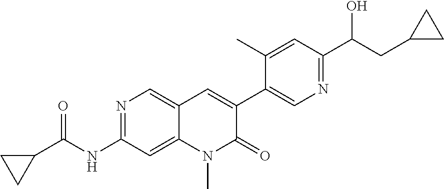 [SMILES: Cc1cc(C(O)CC2CC2)ncc1-c1cc2cnc(NC(=O)C3CC3)cc2n(C)c1=O] |
| 158 | 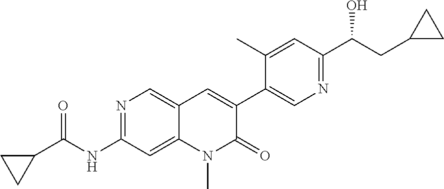 [SMILES: Cc1cc([C@H](O)CC2CC2)ncc1-c1cc2cnc(NC(=O)C3CC3)cc2n(C)c1=O] | 157-1 | 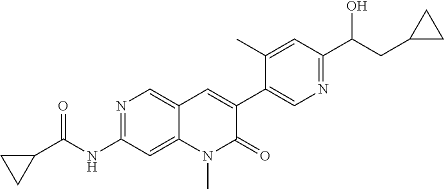 [SMILES: Cc1cc(C(O)CC2CC2)ncc1-c1cc2cnc(NC(=O)C3CC3)cc2n(C)c1=O] |

Although certain compounds described in Table 1 are presented as specific stereoisomers and/or in a non-stereochemical form, it is understood that any or all stereochemical forms, including any enantiomeric or diastereomeric forms, and any tautomers or other forms of any of the compounds of Table 1 are herein described. In some embodiments, the compound described herein is selected from Compound No. 1-158.

This disclosure also includes all salts, such as pharmaceutically acceptable salts, of compounds referred to herein. This disclosure also includes any or all of the stereochemical forms, including any enantiomeric or diastereomeric forms, and any tautomers or other forms, such as N-oxides, solvates, hydrates, or isotopomers, of the compounds described. The present disclosure also includes co-crystals of the compounds described herein. Unless stereochemistry is explicitly indicated in a chemical structure or name, the structure or name is intended to embrace all possible stereoisomers of a compound depicted. In addition, where a specific stereochemical form is depicted, it is understood that other stereochemical forms are also embraced by the invention. All forms of the compounds are also embraced by the invention, such as crystalline or non-crystalline forms of the compounds. Compositions comprising a compound of the invention are also intended, such as a composition of substantially pure compound, including a specific stereochemical form thereof. Compositions comprising a mixture of compounds of the invention in any ratio are also embraced by the invention, including mixtures of two or more stereochemical forms of a compound of the invention in any ratio, such that racemic, non-racemic, enantioenriched and scalemic mixtures of a compound are embraced.

In the descriptions herein, it is understood that every description, variation, embodiment, or aspect of a moiety can be combined with every description, variation, embodiment, or aspect of other moieties the same as if each and every combination of descriptions is specifically and individually listed. For example, every description, variation, embodiment, or aspect provided herein with respect to R¹ of formula (I) may be combined with every description, variation, embodiment, or aspect of X^{a}, R², R³, R⁴, R⁵, R⁶ and/or Ring A, the same as if each and every combination were specifically and individually listed. It is also understood that all descriptions, variations, embodiments or aspects of formula (I), where applicable, apply equally to other formulae detailed herein, and are equally described, the same as if each and every description, variation, embodiment or aspect were separately and individually listed for all formulae. For example, all descriptions, variations, embodiments, or aspects of formula (I), where applicable, apply equally to any of formulae (I-1), (I-2), (IA), (IB), (IC), (ID), (IE), (IF), (IG), (I-a), (I-b), (I-c), (I-d), (I-e), (I-f), and (I-g), detailed herein, and are equally described, the same as if each and every description, variation, embodiment or aspect were separately and individually listed for all formulae.

### III. General Synthetic Methods

The compounds of the present disclosure may be prepared by a number of processes as generally described below and more specifically in the Examples hereinafter (such as the schemes provided in the Examples below). In the following process descriptions, the symbols when used in the formulae depicted are to be understood to represent those groups described above in relation to the formulae herein.

The intermediates described in the following preparations may contain a number of nitrogen, hydroxy, and acid protecting groups such as esters. The variable protecting group may be the same or different in each occurrence depending on the particular reaction conditions and the particular transformations to be performed. The protection and deprotection conditions are well known to the skilled artisan and are described in the literature. See e.g., Greene and Wuts, Protective Groups in Organic Synthesis, (T. Greene and P. Wuts, eds., 2d ed. 1991).

Certain stereochemical centers have been left unspecified and certain substituents have been eliminated in the following schemes for the sake of clarity and are not intended to limit the teaching of the schemes in any way. Furthermore, individual isomers, enantiomers, and diastereomers may be separated or resolved by one of ordinary skill in the art at any convenient point in the synthesis of compounds of the invention, by methods such as selective crystallization techniques or chiral chromatography (See e.g., J. Jacques, et al., “Enantiomers, Racemates, and Resolutions”, John Wiley and Sons, Inc., 1981, and E. L. Eliel and S. H. Wilen, “Stereochemistry of Organic Compounds”, Wiley-Interscience, 1994).

The compounds of the present invention, or a pharmaceutically acceptable salt, solvate, hydrate, or co-crystal thereof, or a mixture of any of the foregoing, may be prepared by a variety of procedures known in the art, some of which are illustrated in the Examples below. The specific synthetic steps for each of the routes described may be combined in different ways, to prepare compounds of the present disclosure, or salts thereof. The products of each step can be recovered by conventional methods well known in the art, including extraction, evaporation, precipitation, chromatography, filtration, trituration, and crystallization. The reagents and starting materials are readily available to one of ordinary skill in the art. Others may be made by standard techniques of organic and heterocyclic chemistry which are analogous to the syntheses of known structurally-similar compounds and the procedures described in the Examples which follow including any novel procedures.

Compounds of formula (I) can be prepared according to Scheme A, Scheme B, Scheme C, or Scheme D, wherein the Ring A moiety, R¹, R², R³, R⁴, R⁵, R⁶ and X^{a} are as defined for formula (I) or any applicable variation thereof as detailed herein.

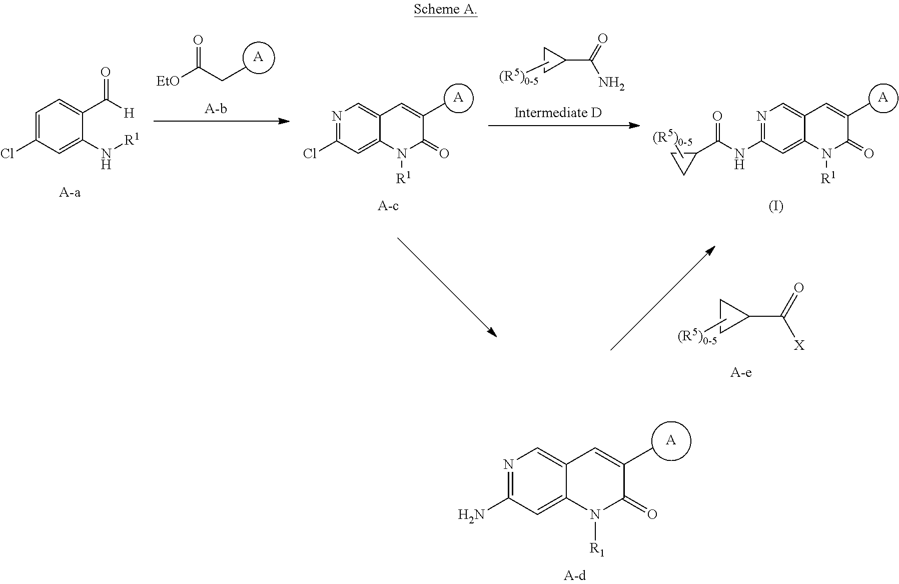

Compounds of formula (I) may be prepared according to the general synthetic scheme shown in Scheme A. In Scheme A, acylation of nicotinaldehydes of general formula A-a with Ring A-substituted esters of formula A-b yields the corresponding coupled amides which then undergo cyclization to yield naphthyridinones of general formula A-c. The naphthyridinones of general formula A-c can be reacted with optionally R⁵-substituted cyclopropylamides of general formula Intermediate D to give compounds of formula (I). Alternatively, naphthyridinones of general formula A-c may be aminated to compounds of general formula A-d, and subsequently reacted with optionally R⁵-substituted cyclopropyl carboxylic acids (X is OH) or cyclopropyl acid chlorides (X is Cl) of general formula A-e to yield compounds of formula (I).

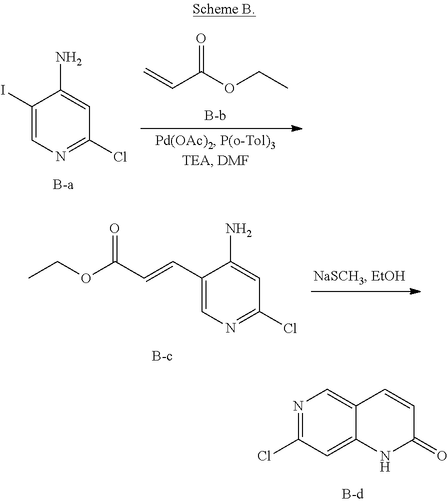

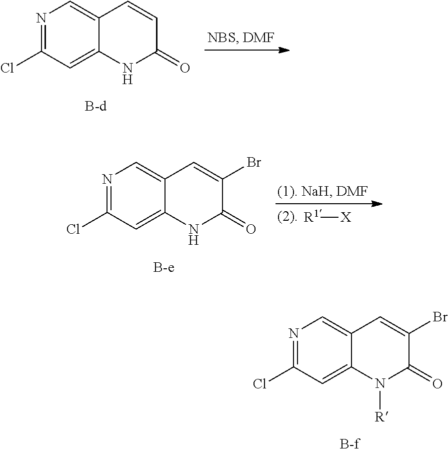

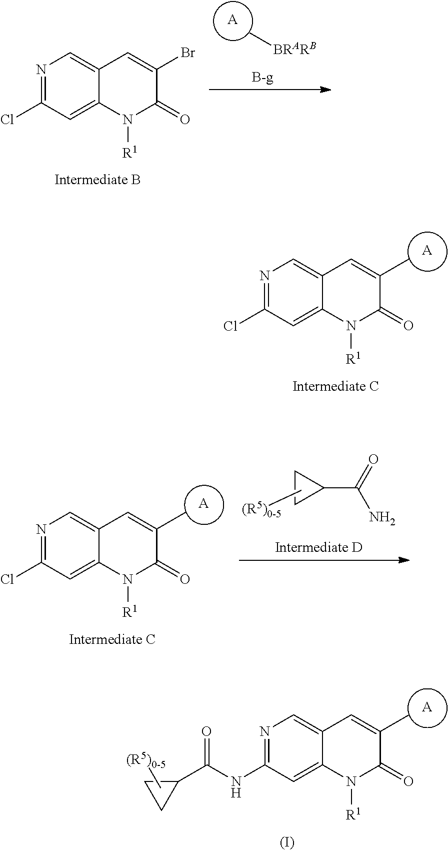

Compounds of formula (I) may also be prepared according to the general synthetic scheme shown in Scheme B. In Scheme B, intermediate B-a is coupled with compound B-b to afford compound B-c, which is subsequently converted to compound B-d. Compound B-d is then brominated with N-bromosuccinimide to afford compound B-e. Compound B-e can be deprotonated with sodium hydride to allow further substitution of the annular nitrogen (e.g., alkylation with an R^{1′} substrate having a suitable leaving group X, such as methyl iodide) to afford Intermediate B-f, wherein R^{1′} is equivalent to R¹ except that R^{1′} does not include hydrogen. Either compound B-e or compound B-f is used as Intermediate B in subsequent reactions to be coupled with a suitable Ring A-substituted boronic acid derivative B-g, wherein R^{A} and R^{B} are independently selected from the group consisting of halogen, OH, and O—(C₁-C₆ alkyl), or R^{A} and R^{B} are taken together with the boron atom to which they are attached to form a 5-10 membered heterocycle, to afford Intermediate C. Intermediate C can then be reacted with Intermediate D to give compounds of formula (I).

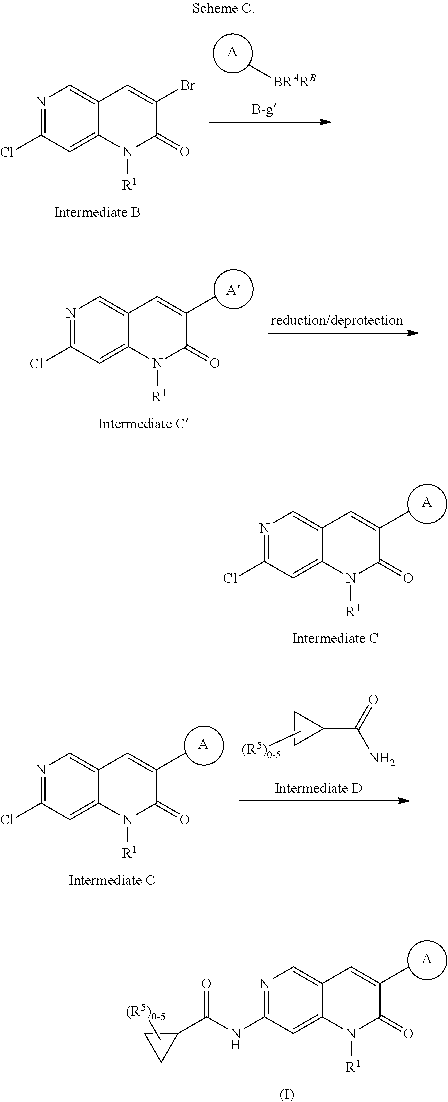

Compounds of formula (I) may also be prepared according to the general synthetic scheme shown in Scheme C. In Scheme C, intermediate B can be coupled with a suitable Ring A′-substituted boronic acid derivative B-g′, wherein Ring A′ is a precursor to Ring A, and wherein R^{A} and R^{B} are independently selected from the group consisting of halogen, OH, and O—(C₁-C₆ alkyl), or R^{A} and R^{B} are taken together with the boron atom to which they are attached to form a 5-10 membered heterocycle, to afford Intermediate C′. Intermediate C′ can be reduced or deprotected to yield Intermediate C, which can then be reacted with Intermediate D to give compounds of formula (I).

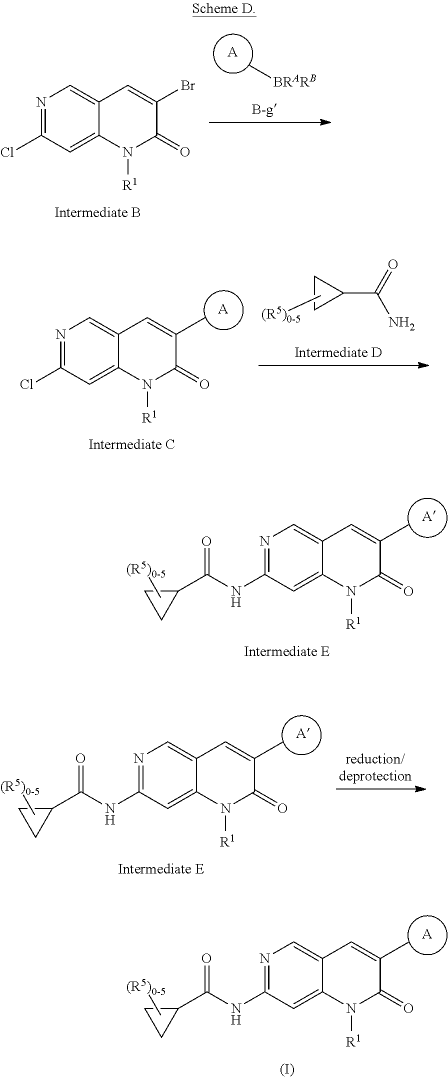

Compounds of formula (I) may also be prepared according to the general synthetic scheme shown in Scheme D. In Scheme D, intermediate B can be coupled with a suitable Ring A′-substituted boronic acid derivative B-g′, wherein Ring A′ is a precursor to Ring A, and wherein R^{A} and R^{B} are independently selected from the group consisting of halogen, OH, and O—(C₁-C₆ alkyl), or R^{A} and R^{B} are taken together with the boron atom to which they are attached to form a 5-10 membered heterocycle, to afford Intermediate C′. Intermediate C′ can be reacted with Intermediate D yield Intermediate E, which can then be reduced or deprotected to give compounds of formula (I).

It should be recognized that the present disclosure also provides for any intermediates of the compounds and methods for synthesizing the compounds as described herein. In another aspect, provided herein are general intermediates as described in any one of Schemes A through D above, or compound-specific intermediates as described in the examples below. It should be further recognized that the present disclosure also provides for synthetic methods comprising any individual step or combination of individual process steps, or compositions of synthetic intermediates and/or reaction products as described herein.

### IV. Pharmaceutical Compositions and Formulations

Any of the compounds described herein may be formulated as a pharmaceutically acceptable composition.

Pharmaceutical compositions of any of the compounds detailed herein are embraced by this disclosure. Thus, the present disclosure includes pharmaceutical compositions comprising a compound as detailed herein, or a pharmaceutically acceptable salt, solvate, hydrate, or co-crystal thereof, or a mixture of any of the foregoing, and a pharmaceutically acceptable carrier or excipient. In one aspect, the pharmaceutically acceptable salt is an acid addition salt, such as a salt formed with an inorganic or organic acid. Pharmaceutical compositions may take a form suitable for oral, buccal, parenteral, nasal, topical or rectal administration or a form suitable for administration by inhalation.

A compound as detailed herein may in one aspect be in a purified form and compositions comprising a compound in purified forms are detailed herein. Compositions comprising a compound, or a pharmaceutically acceptable salt, solvate, hydrate, or co-crystal thereof, or a mixture of any of the foregoing, as detailed herein are provided, such as compositions of substantially pure compounds. In some embodiments, a composition containing a compound, or a pharmaceutically acceptable salt, solvate, hydrate, or co-crystal thereof, or a mixture of any of the foregoing, as detailed herein is in substantially pure form. In one variation, “substantially pure” intends a composition that contains no more than 35% impurity, wherein the impurity denotes a compound other than the compound comprising the majority of the composition or a salt thereof. For example, a composition of a substantially pure compound selected from a compound of Table 1 intends a composition that contains no more than 35% impurity, wherein the impurity denotes a compound other than the compound of Table 1. In one variation, a composition of substantially pure compound, or a pharmaceutically acceptable salt, solvate, hydrate, or co-crystal thereof, or a mixture of any of the foregoing, is provided wherein the composition contains no more than 25% impurity. In another variation, a composition of substantially pure compound, or a pharmaceutically acceptable salt, solvate, hydrate, or co-crystal thereof, or a mixture of any of the foregoing, is provided wherein the composition contains or no more than 20% impurity. In still another variation, a composition of substantially pure compound, or a pharmaceutically acceptable salt, solvate, hydrate, or co-crystal thereof, or a mixture of any of the foregoing, is provided wherein the composition contains or no more than 10% impurity. In a further variation, a composition of substantially pure compound, or a pharmaceutically acceptable salt, solvate, hydrate, or co-crystal thereof, or a mixture of any of the foregoing, is provided wherein the composition contains no more than 5% impurity. In another variation, a composition of substantially pure compound, or a pharmaceutically acceptable salt, solvate, hydrate, or co-crystal thereof, or a mixture of any of the foregoing, is provided wherein the composition contains no more than 3% impurity. In still another variation, a composition of substantially pure compound, or a pharmaceutically acceptable salt, solvate, hydrate, or co-crystal thereof, or a mixture of any of the foregoing, is provided wherein the composition contains no more than 1% impurity. In a further variation, a composition of substantially pure compound, or a pharmaceutically acceptable salt, solvate, hydrate, or co-crystal thereof, or a mixture of any of the foregoing, is provided wherein the composition contains no more than 0.5% impurity. In yet other variations, a composition of substantially pure compound means that the composition contains no more than 15%, no more than 10%, no more than 5%, no more than 3%, or no more than 1% impurity, which impurity may be the compound in a different stereochemical form. For instance, and without limitation, a composition of substantially pure (S) compound means that the composition contains no more than 15% or no more than 10% or no more than 5% or no more than 3% or no more than 1% of the (R) form of the compound.

In one variation, the compounds herein are synthetic compounds prepared for administration to an individual. In another variation, compositions are provided containing a compound in substantially pure form. In another variation, the present disclosure embraces pharmaceutical compositions comprising a compound detailed herein and a pharmaceutically acceptable carrier. In another variation, methods of administering a compound are provided. The purified forms, pharmaceutical compositions and methods of administering the compounds are suitable for any compound or form thereof detailed herein. In some embodiments, the compounds and compositions as provided herein are sterile. Methods for sterilization known in the art may be suitable for any compounds or form thereof and compositions thereof as detailed herein.

A compound detailed herein, or a pharmaceutically acceptable salt, solvate, hydrate, or co-crystal thereof, or a mixture of any of the foregoing, may be formulated for any available delivery route, including an oral, mucosal (e.g., nasal, sublingual, vaginal, buccal or rectal), parenteral (e.g., intramuscular, subcutaneous or intravenous), topical or transdermal delivery form. A compound, or a pharmaceutically acceptable salt, solvate, hydrate, or co-crystal thereof, or a mixture of any of the foregoing, may be formulated with suitable carriers to provide delivery forms that include, but are not limited to, tablets, caplets, capsules (such as hard gelatin capsules or soft elastic gelatin capsules), cachets, troches, lozenges, gums, dispersions, suppositories, ointments, cataplasms (poultices), pastes, powders, dressings, creams, solutions, patches, aerosols (e.g., nasal spray or inhalers), gels, suspensions (e.g., aqueous or non-aqueous liquid suspensions, oil-in-water emulsions or water-in-oil liquid emulsions), solutions and elixirs.

A compound detailed herein, or a pharmaceutically acceptable salt, solvate, hydrate, or co-crystal thereof, or a mixture of any of the foregoing, can be used in the preparation of a formulation, such as a pharmaceutical formulation, by combining the compound or compounds, or a pharmaceutically acceptable salt, solvate, hydrate, or co-crystal thereof, or a mixture of any of the foregoing, with a pharmaceutically acceptable carrier. Depending on the therapeutic form of the system (e.g., transdermal patch vs. oral tablet), the carrier may be in various forms. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants. Formulations comprising the compound may also contain other substances which have valuable therapeutic properties. Pharmaceutical formulations may be prepared by known pharmaceutical methods. Suitable formulations can be found, e.g., in Remington's Pharmaceutical Sciences, Mack Publishing Company, Philadelphia, PA, 20th ed. (2000), which is incorporated herein by reference.

A compound detailed herein, or a pharmaceutically acceptable salt, solvate, hydrate, or co-crystal thereof, or a mixture of any of the foregoing, may be administered to individuals in a form of generally accepted oral compositions, such as tablets, coated tablets, and gel capsules in a hard or in soft shell, emulsions or suspensions. Examples of carriers, which may be used for the preparation of such compositions, are lactose, corn starch or its derivatives, talc, stearate or its salts, etc. Acceptable carriers for gel capsules with soft shell are, for instance, plant oils, wax, fats, semisolid and liquid poly-ols, and so on. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants.

Any of the compounds, or a pharmaceutically acceptable salt, solvate, hydrate, or co-crystal thereof, or a mixture of any of the foregoing, described herein can be formulated in a tablet in any dosage form described, for example, a compound as described herein, or a pharmaceutically acceptable salt, solvate, hydrate, or co-crystal thereof, or a mixture of any of the foregoing, can be formulated as a 10 mg tablet.

Compositions comprising a compound, or a pharmaceutically acceptable salt, solvate, hydrate, or co-crystal thereof, or a mixture of any of the foregoing, provided herein are also described. In one variation, the composition comprises a compound, or a pharmaceutically acceptable salt, solvate, hydrate, or co-crystal thereof, or a mixture of any of the foregoing, and a pharmaceutically acceptable carrier or excipient. I n another variation, a composition of substantially pure compound, or a pharmaceutically acceptable salt, solvate, hydrate, or co-crystal thereof, or a mixture of any of the foregoing, is provided. In some embodiments, the composition is for use as a human or veterinary medicament. In some embodiments, the composition is for use in a method described herein. In some embodiments, the composition is for use in the treatment of a disease or disorder described herein.

Compositions formulated for co-administration of a compound provided herein and one or more additional pharmaceutical agents are also described. The co-administration can be simultaneous or sequential in any order. A compound provided herein may be formulated for co-administration with the one or more additional pharmaceutical agents in the same dosage form (e.g., single tablet or single i.v.) or separate dosage forms (e.g., two separate tablets, two separate i.v., or one tablet and one i.v.). Furthermore, co-administration can be, for example, 1) concurrent delivery, through the same route of delivery (e.g., tablet or i.v.), 2) sequential delivery on the same day, through the same route or different routes of delivery, or 3) delivery on different days, through the same route or different routes of delivery.

### V. Methods of Use

Compounds and compositions detailed herein, such as a pharmaceutical composition containing a compound of formula (I) or any variation thereof provided herein, or a pharmaceutically acceptable salt, solvate, hydrate, or co-crystal thereof, or a mixture of any of the foregoing, and a pharmaceutically acceptable carrier or excipient, may be used in methods of administration and treatment as provided herein. The compounds and compositions may also be used in in vitro methods, such as in vitro methods of administering a compound or composition to cells for screening purposes and/or for conducting quality control assays.

In one aspect, provided herein is are methods of a method of treating a cancer or neoplastic disease in a human in need thereof. In some embodiments, provided herein are methods of treating a disease or disorder mediated by a RAF kinase. In other embodiments, provided herein are methods of treating a disease or disorder mediated by Bcr-Abl tyrosine kinase.

### RAF Kinase Inhibition

In one aspect, provided herein is a method of inhibiting ARAF, BRAF and CRAF enzymatic activity in a cell, comprising exposing the cell with an effective amount of a compound of formula (I) as described herein, or a pharmaceutically acceptable salt, solvate, hydrate, or co-crystal thereof, or a mixture of any of the foregoing, or a pharmaceutical composition comprising a compound of formula (I) as described herein, or a pharmaceutically acceptable salt, solvate, hydrate, or co-crystal thereof, or a mixture of any of the foregoing.

The compounds and compositions described herein may be used in a method of treating a disease or disorder mediated by ARAF, BRAF, or CRAF kinase activity. In some embodiments, the compound or composition is administered according to a dosage described herein.

In some embodiments, provided herein is a method for treating a disease or disorder mediated by RAF kinase activity comprising administering to an individual in need of treatment an effective amount of a compound of formula (I) or any variation thereof, or a pharmaceutically acceptable salt, solvate, hydrate, or co-crystal thereof, or a mixture of any of the foregoing. In some embodiments, the disease or disorder is a cancer or neoplastic disease.

In still yet another aspect, provided herein is a method of treating a cancer or neoplastic disease in a human in need thereof, comprising administering to the human a compound of formula (I) as described herein, or a pharmaceutically acceptable salt, solvate, hydrate, or co-crystal thereof, or a mixture of any of the foregoing, or a pharmaceutical composition comprising a compound of formula (I) as described herein, or a pharmaceutically acceptable salt, solvate, hydrate, or co-crystal thereof, or a mixture of any of the foregoing.

### RAF Kinase Family

In vertebrates, RAF is comprised of a family of three genetically distinct serine/threonine protein kinases designated ARAF, BRAF and CRAF (sometimes referred to as RAF-1). These family members are highly conserved at the primary sequence level (75% amino acid identity across their entire protein sequence and >87% identity within their respective kinase domains) and exhibit the same overall domain architecture. ARAF, BRAF and CRAF are ubiquitously and differentially expressed across all cell and tissue types. As such, they collectively serve as an essential signaling node of the Ras/Raf/MEK/ERK (MAPK) pathway. Importantly, a substantial proportion of all cancers are driven by genetic alterations in either the RTKs or a particular member of the MAPK pathway especially HRAS, KRAS, NRAS and the RAF isoforms. that then drive aberrant activation of the pathway. Therefore, as an essential node in the MAPK pathway, the RAF kinases represent an important therapeutic intervention point for the treatment of a variety of malignancies whose dysregulated growth and survival rely upon MAPK signaling. Accordingly, multiple RAF kinase inhibitors have been approved for specific indications including melanoma and NSCLC and numerous additional inhibitors are currently undergoing clinical investigation for a variety of other malignancies.

### RAF Function

Upon ligand binding, RTKs homo- or hetero oligomerize with other receptors and auto-phosphorylate key tyrosine residues in trans. These phosphorylated residues then serve as docking sites for downstream effectors, especially adapter proteins involved in the recruitment and activation of RAS (H-, K- and N-RAS) such as Grb2 and SOS, respectively. Activated GTP-bound RAS now binds and recruits RAF thereby inducing conformational changes in the latter to induce its dimerization and concomitant activation. RAF then binds and phosphorylates/activates MEK which then phosphorylates/activates ERK. Activated ERK then redistributes to the cytoplasm, the cytoskeleton and the nucleus to control cell growth/division, differentiation and survival.

### RAF Structure and Regulation

Grossly, the primary structure of RAF can be divided into two domains; an N-terminal regulatory domain and a C-terminal kinase domain (KD) connected by a linker region. The regulatory domain contains multiple elements including a RAS-binding domain (RBD) followed immediately downstream by a Cysteine-Rich Domain (CRD). A key phosphorylation site resides within the linker region and another at the extreme C-terminus downstream of the KD. In its inactive conformation, RAF is located in the cytoplasm in a monomeric, dual-phosphorylated, autoinhibited state. This autoinhibition is mediated via two cooperative mechanisms: (1) direct interaction between the RBD and the KD and (2) 14-3-3 protein dimers that simultaneously interact with the two phosphorylated residues flanking the KD. The combination of these interactions effectively binds up the KD into the inactive conformation. Upon RAS engagement via interactions with both the RBD and CRD, the RBD-KD interaction is effectively disrupted exposing the phosphorylation site within the linker region to phosphatase action via the MRAS/SHOC2/PP1 complex. Subsequent dephosphorylation of this residue abrogates intramolecular 14-3-3 binding thereby fully relieving autoinhibition and exposing residues critical for interaction with the plasma membrane. RAS-bound hemi-phosphorylated RAF can now dimerize with another RAF protein (homo- or heterodimerization) via intermolecular interactions between their respective KDs as well as 14-3-3 cross-linking between the two adjacent phosphorylated residues at the C-terminus of each protomer. Importantly, this fully active RAF complex functions as an obligate dimer to both bind to and activate MEK, ultimately driving ERK activation to complete the signaling cascade.

### RAF Mutations and Cancer

Given their critical involvement in the RTK/RAS/RAF/MEK/ERK pathway, it should be no surprise each of the RAF isoforms are bonafide proto-oncogenes. Accordingly, a variety of mutations have been identified in ARAF, BRAF and CRAF that have been functionally linked to tumor formation. Importantly, these mutations fall into distinct classes with discrete mechanisms of kinase activation.

BRAF is the most commonly mutated RAF isoform with alterations reported in approximately 8% of all solid tumors. Melanomas harbor the greatest proportion of BRAF mutations with 40-50% prevalence followed by thyroid, colorectal (CRC) and non-small cell lung cancers (NSCLC). These mutations can be divided into three distinct functional classes based upon how they elicit aberrant activation of RAF kinase activity. Class I mutations render the kinase constitutively active and independent of the requirement for RAS binding or dimerization with another RAF isoform. These mutations are unique to BRAF and are associated with highly specific alterations within the 600^{th} codon leading to the conversion of a valine residue to an aspartate, glutamate, lysine or arginine (V600D/E/K/R). Class II mutations drive aberrant kinase activation by conferring constitutive RAS-independent RAF-dimerization without adversely impacting the intrinsic kinase activity of the mutant. These mutations can be further subdivided into 3 subclasses according to which region within the kinase domain the alteration occurs (designated as Class IIa and IIb) or the formation of a kinase fusion arising from a chromosome translocation event (designated Class IIc) whereby the negative regulatory RBD and CRD domains are removed by deletion and replaced with the fusion partner. The class II mutations include the following: G464V, G469A, G469V, G469R, E586K, K601E, K601N, L597R, L597S, L597Q) These mutations are most common in NSCLC and CRC. Finally, Class III mutants confer enhanced RAS-dependent RAF dimerization to drive pathway activation. These mutations substantially attenuate the intrinsic kinase activity of the mutant such that transactivation of the wildtype RAF dimerization partner is key to aberrant pathway activation. Accordingly, other genetic alterations leading to RAS activation are often found co-occurring with these Class III mutations to facilitate dimerization. The class III mutations include the following: G466R, G466A, G466E, G466V, N581I, N581S, D594E, D594G, D594N, G596C, G596R.

Compared to BRAF, the prevalence of oncogenic mutations within CRAF are relatively rare and found sporadically across a wide array of cancers including melanoma, NSCLC, pancreatic carcinoma, glioma, colorectal and hematological malignancies. There are two distinct mutation types that have been reported for CRAF. The first mutation type consists of point mutations that reside within the linker region effectively disrupting 14-3-3 binding to the linker domain phosphorylation site and conferring a more open confirmation that is now accessible to phosphatase action and subsequent dimerization/activation. These mutations include P261L and P261A. The second CRAF mutation type is analogous to the Class II mutations in BRAY. Specifically, there are reports of point mutations found in regions of the kinase domain analogous to the Class IIa and IIb BRAY mutants in CRAF across multiple cancer types, especially melanomas. These mutations include E478K, R391W, R391S and T491I as well as certain mutations that are also found in a subset of RASopathies; a cluster of diverse genetic diseases whose underlying etiology appears to derive from chronic MAPK pathway activation. There are also multiple reports of Class IIc mutations in RAF (CRAF fusions), which, like the BRAF fusions, possess fusion partners that effectively replace the RBD and CRD domains to relieve autoinhibition and drive dimerization and activation.

To date, only one oncogenic mutation at codon 214 in ARAF has been reported. This mutation results in either a cysteine or phenylalanine for serine substitution (S214C/F) and has been identified in multiple NSCLC patients with an approximate prevalence of 0.5%. Given that no additional oncogenic mutations were identified in these tumors, it is likely that the ARAF mutants are the oncogenic drivers in these cancers. Accordingly, in vitro characterization of cell lines engineered to express an S214F ARAF mutant revealed that the mutant induces MAPK pathway activation and markedly enhances colony formation (a hallmark activity of an oncogene) in a kinase-dependent manner. Given that S214 is the linker region phosphorylation site critical for 14-3-3 binding, it is likely conferring constitutive activation in a manner very similar to the CRAF point mutants described above.

### RAF Kinase Inhibitors

Given the strong link between genetic alterations in components of the MAPK pathway and the development of cancer in a wide array of tumor types, this pathway represents a key opportunity for the development of targeted therapies to control these proliferative diseases. In particular, inhibitors directed against the RAF family should offer an important treatment option for patients harboring RAF kinase activating mutations found in a number of cancer types including those of the skin, thyroid and lung. Accordingly, over the last 2 decades, numerous small molecule RAF inhibitors have been discovered and several of these have advanced into the clinic and gone on to full regulatory approval. The vast majority of these compounds are ATP-competitive small molecule inhibitors and bind in the kinase active site. They are divided into three types, dependent upon the specific structural conformation they induce within the kinase upon binding. These inhibitor types are designated 1, 1.5 and 2.

### Type 1 Inhibitors

Type 1 inhibitors bind in the active or ‘closed’ form of the kinase domain which is largely defined by the relative inward orientation of the C-helix and the ‘DFG’ loop which both comprise key structural and functional elements of the active site. This binding mode is designated C-helix-in/DFG-in. These compounds make key interactions with what is known as the hinge (the flexible linker between the amino and carboxyl terminal lobes of the kinase domain) as well as the pocket that normally accommodates the adenine ring of ATP. SB590885 and GDC-0879 are two literature examples of type I RAF inhibitors. Both were demonstrated to be almost exclusively active in BRAF Class 1 mutant cell contexts both in vitro and in vivo. Despite this promising activity, to date, no type I inhibitors have entered clinical development.

Type 2 inhibitors

Type 2 inhibitors bind to the kinase domain in an open conformation in which the DFG-loop is oriented in an outward or inactive position. This conformation exposes an allosteric, hydrophobic pocket adjacent to the ATP binding site that can be exploited to gain further enhancements in potency and selectivity via hydrogen bonding, Van der Waals and hydrophobic interactions. Accordingly, Type 2 inhibitors consist of functionalities that interact with both the hinge region as well as the allosteric pocket leaving the C-helix in an inward undisturbed orientation. Accordingly, this conformation is denoted as C-helix-in/DFG-out. In the literature, there exist a number of examples of Type 2 RAF inhibitors including several that have undergone clinical evaluation. Unlike the Type 1 inhibitor examples, these molecules as a class are more broadly active, exhibiting activity across a range of mutant contexts including RAS (KRAS, NRAS, HRAS), BRAF (Class 1, II and III) and CRAF. To date, multiple Type 2 inhibitors have entered into clinical development for patients harboring genetic alterations in the MAPK pathway. Importantly, several of these agents have demonstrated clinical activity in both Class I mutant BRAF and RAS mutant contexts. However, the activity has been limited and no Type 2 RAF inhibitor is currently approved for any indication.

### Type 1.5 Inhibitors

Type 1.5 inhibitors bind to both the hinge region as well as the space typically occupied by the adenine moiety of ATP in much the same way as the Type 1 RAF inhibitors. What distinguishes the Type 1.5 inhibitors is that they take advantage of additional interactions at the back of the ATP binding pocket made accessible by the relatively small threonine gatekeeper residue found in all RAF isoforms (T382 in ARAF, T529 in BRAF and T421 in CRAF). Importantly, these back-pocket interactions alter the conformation of the C-helix, forcing it into an outward conformation while the DFG loop is oriented in its active or ‘in’ conformation. This conformation is denoted as C-helix-out/DFG-in. This conformation exerts a significant impact on the affinity of inhibitor for the second protomer of the RAF dimer rendering it markedly less able to bind inhibitor. Consequently, Type 1.5 inhibitors are highly active against BRAF Class I mutants that signal as monomers versus other MAPK pathway mutant contexts and the wildtype state where RAF signals as an obligate dimer. To date, 3 Type 1.5 inhibitors have been approved for the treatment of malignant melanomas harboring Class 1 BRAF mutations: vemurafenib, dabrafenib and encorafenib.

### Paradoxical Activation

As described above, ARAF, BRAF and CRAF are primarily regulated at the structural level in which various intra- and inter-molecular protein-protein interactions define both their localization and activity state. Accordingly, the structural changes induced with inhibitor binding exert biological effects beyond simple inhibition of kinase activity and these effects can differ depending upon the genetic context of the cells or tissues being exposed to inhibitor. In addition, dependent upon the inhibitor type, these effects are distinct, having important implications regarding safety as well as sensitivity and resistance to inhibitor treatment.

In normal cells and tissues in which the RAF isoforms are unmutated, inhibitor binding actually enhances signaling flux through the MAPK pathway in what is known as paradoxical activation. This effect derives from one or more of four distinct yet interdependent mechanisms; (1) attenuation of inhibitory auto-phosphorylation in the linker region, (2) interruption of kinase domain interactions, (3) enhancement of binding to GTP-bound RAS at the plasma membrane and (4) transactivation of the second protomer of the RAF dimer. The first 3 of these mechanisms collectively drive enhanced RAF protomer dimerization and therefore enhance downstream signaling. The fourth mechanism involves inhibitor binding to the first protomer of the RAF dimer to induce a C-helix out conformation that effectively locks the conformation of the active site of the second protomer to the active C-helix-in conformation thereby inducing both its activation and markedly reducing its affinity for inhibitor (negative allostery). The extent and magnitude of activation is dependent upon which of these mechanisms are induced by inhibitor binding and this is ultimately dictated by the binding mode of the inhibitor. Accordingly, Type 1, 2 and 1.5 inhibitors all engage the first 3 mechanisms to induce paradoxical activation. Only the Type 1.5 inhibitors engage the fourth mechanism to further enhance paradoxical activation.

### Therapeutic Resistance

Clinical resistance to Type 1.5 and Type 2 inhibitors has been observed, but with distinct mechanisms of action. Patients with BRAF Class I mutant melanoma that become refractory to or relapse on Type 1.5 inhibitor therapies often exhibit mutations that drive RAF dimerization. These alterations typically involve RAF amplification/overexpression or RAS mutations but can also include aberrant alternative splicing events that remove the RBD and CRD and effectively remove the blockade to dimerization. When the Class I mutant BRAF protomer acts in the context of a dimer rather than its typical monomeric state, it is much less sensitive to Type 1.5 inhibitor treatment. This is due largely to the inhibitor's impact on the C-helix which, as mentioned in the previous section not only results in transactivation of the unoccupied protomer but it also renders this protomer markedly less able to bind inhibitor such that super-clinical concentrations of inhibitor are required to significantly attenuate MAPK pathway signaling in the resistant tumor. Given the relatively limited clinical data available for the Type 2 RAF inhibitors, only one mechanism of therapeutic resistance has been reported thus far. In the case of belvarafenib, multiple patients that relapsed on therapy exhibited alterations in ARAF. These mutations reside within the kinase domain active site and rendered the kinase resistant not only to belvarafenib but a panel of Type 2 inhibitors.

### Mutant Coverage

Because the type 1.5 inhibitors are not effective at inhibiting RAF activity in the context of a dimer, they are only effective at inhibiting Class I BRAF mutants that signal as monomers. Because Type 2 inhibitors can inhibit both monomeric and dimeric RAF, they are able to inhibit Class II and III BRAF mutants that signal as obligate dimers in addition to the Class I mutants.

### Clinical Safety

Paradoxical activation is known to adversely impact the tolerability of these inhibitors in patients, thereby limiting their clinical utility. As mentioned previously, Type 1.5 inhibitors markedly induce paradoxical activation in normal tissues by binding RAF dimers and transactivating the second unbound protomer. Accordingly, in the clinic, Type 1.5 inhibitor treatment is associated with multiple adverse events associated with aberrant MAPK pathway activation particularly involving the skin such as palmoplantar erythrodysaesthesia syndrome and proliferative skin lesions including keratoacanthomas and cutaneous squamous cell carcinomas. MEK inhibitors have been successfully deployed in combination with Type 1.5 RAF inhibitors to effectively manage these toxicities. Specifically, vemurafenib, dabrafenib and encorafenib have been approved in combination with cobimetinib, trametinib and binimetinib, respectively, for patients with BRAF Class I mutant metastatic melanoma. Not only have these combinations improved tolerability by attenuating paradoxical activation in normal tissues but they have also improved therapeutic benefit both in terms of overall response rate and long term survival.

In contrast, Type 2 inhibitors can bind and inhibit both protomers equally thereby significantly attenuating paradoxical activation and driving full MAPK inhibition, even in normal unmutated tissues. Consequently, the toxicities associated with Type 2 inhibitors are more in keeping with those elicited by MEK inhibitors.

In still yet another aspect, provided herein is a method of treating a cancer or neoplastic disease in a human in need thereof, comprising administering to the human a compound of formula (I) as described herein, or a pharmaceutically acceptable salt, solvate, hydrate, or co-crystal thereof, or a mixture of any of the foregoing, or a pharmaceutical composition comprising a compound of formula (I) as described herein, or a pharmaceutically acceptable salt, solvate, hydrate, or co-crystal thereof, or a mixture of any of the foregoing, wherein the cancer or neoplastic disease is associated with one or more genetic alterations that engender elevated RAS/RAF/MEK/ERK pathway activation. In some embodiments, the cancer or neoplastic disease is associated with one or more genetic alterations in KRAS, NRAS, HRAS, ARAF, BRAF or CRAF. In some embodiments, the cancer or neoplastic disease is associated with one or more mutations in KRAS selected from the group consisting of G12D, G12V, G12C, G12S, G12R, G12A, G13D, G13C, G13R, Q61H, Q61K, Q61L, Q61P, Q61R and Q61E. In some embodiments, the cancer or neoplastic disease is associated with one or more mutations in NRAS selected from the group consisting of G12D, G12S, G12C, G12V, G12A, G13D, G13R, G13V, G13C, G13A, G13S, G61R, Q61K Q61H, and G61L. In some embodiments, the cancer or neoplastic disease is associated with one or more mutations in HRAS selected from the group consisting of G12V, G12S, G12D, G12C, G12R, G12A, G13R, G13V, G13D, G13S, G13C, Q61R, Q61L, Q61K, and Q61H. In some embodiments, the cancer or neoplastic disease is associated with one or more mutations in ARAF selected from the group consisting of S214C and S214F. In some embodiments, the cancer or neoplastic disease is associated with one or more mutations in BRAF selected from the group consisting of Class I, Class IIa, Class IIb, Class IIc, and Class III mutations. In some embodiments, the cancer or neoplastic disease is associated with one or more mutations in CRAF selected from the group consisting of P261A, P261L, E478K, R391W, R391S and T491I, or is associated with a CRAF fusion. In other embodiments, the cancer or neoplastic disease is associated with one or more genetic lesions resulting in the activation of one or more receptor tyrosine kinases (RTKs). In some embodiments, the one or more genetic lesions is a point mutation, a fusion or any combination thereof. In some embodiments, the one or more receptor tyrosine kinase is selected from the group consisting of ALK, EGFR, ERBB2, LTK, MET, NTRK, RET, and ROS1.

The compounds and compositions of the present disclosure may be suitable for treatment of certain subtypes of cancer or neoplastic diseases, which may also be associated with mutations in KRAS, NRAS, HRAS, ARAF, BRAF or CRAF. In some embodiments, the cancer is a solid tumor or a hematological malignancy. In some embodiments, the cancer is melanoma, lung cancer, pancreatic carcinoma, glioma, colorectal carcincoma, chronic myeloid leukemia (CML), acute myeloid leukemia (AML), or acute lymphoblastic leukemia (ALL). In certain embodiments, the lung cancer is non-small cell lung cancer (NSCLC). In one aspect, provided herein is a method of treating a solid tumor or a hematological malignancy, comprising administering to the human a compound of formula (I) as described herein, or a pharmaceutically acceptable salt, solvate, hydrate, or co-crystal thereof, or a mixture of any of the foregoing, or a pharmaceutical composition comprising a compound of formula (I) as described herein, or a pharmaceutically acceptable salt, solvate, hydrate, or co-crystal thereof, or a mixture of any of the foregoing. In some embodiments, the solid tumor or hematological malignancy is melanoma, lung cancer, pancreatic carcinoma, glioma, colorectal carcincoma, chronic myeloid leukemia (CML), acute myeloid leukemia (AML), or acute lymphoblastic leukemia (ALL). In certain embodiments, the lung cancer is non-small cell lung cancer (NSCLC).

In some embodiments of the present aspect, the cancer is a refractory cancer. In certain embodiments of the foregoing, the refractory cancer is associated with a genetic alteration or alterations in KRAS (including mutants G12D, G12V, G12C, G12S, G12R, G12A, G13D, G13C, G13R, Q61H, Q61K, Q61L, Q61P, Q61R and Q61E), NRAS (including mutants G12D, G12S, G12C, G12V, G12A, G13D, G13R, G13V, G13C, G13A, G13S, G61R, Q61K Q61H, G61L), HRAS (including mutants G12V, G12S, G12D, G12C, G12R, G12A, G13R, G13V, G13D, G13S, G13C, Q61R, Q61L, Q61K, Q61H), BRAF (including gene amplification, class II and III mutants [including G464V, G469A, G469V, G469R, E586K, K601E, K601N, G466R, G466A, G466E, G466V, N581I, N581S, D594E, D594G, D594N, G596C, G596R, L597R, L597S, L597Q], BRAF fusions or alternative splicing events that result in the loss of BRAF gene exons 4-10, 4-8, 2-8 or 2-10), RTKs (including ALK, EGFR, ERBB2, LTK, MET, NTRK, RET, ROS1). In still further embodiments of the foregoing, the refractory cancer may be combined with any preceding embodiments of the present aspect, the method further comprises administering one or more pharmaceutical agents including anti-microtubular therapies, topoisomerase inhibitors, alkylating agents, nucleotide synthesis inhibitors, DNA synthesis inhibitors, protein synthesis inhibitors, developmental signaling pathway inhibitors, pro-apoptotic agents, RTK inhibitors (including inhibitors against ALK, EGFR, ERBB2, LTK, MET, NTRK, RET, ROS1), RAF inhibitors representing alternative binding modes (such as Type 1.5 or Type II), MEK1/2 inhibitors, ERK1/2 inhibitors, RSK1/2/3/4 inhibitors, AKT inhibitors, TORC1/2 inhibitors, DNA damage response pathway inhibitors (including ATM, ATR), PI3K inhibitors and/or radiation.

In some embodiments of the present aspect, the cancer is a refractory BRAF Class I mutant cancer. In some embodiments, the refractory BRAF Class I mutant cancer is associated with a point mutation selected from the group consisting of V600D, V600E, V600K, and V600R. In certain embodiments of the foregoing, the refractory cancer is associated with a genetic alteration in KRAS, NRAS, HRAS or BRAF that drives BRAF dimerization and confers resistance to approved Type 1.5 inhibitors (including vemurafenib, dabrafenib and encorafenib) both alone and in the context of MEK inhibitor (including cobimetinib, trametinib and binimetinib) combinations. In some embodiments, the refractory cancer is associated with one or more mutations in KRAS selected from the group consisting of G12D, G12V, G12C, G12S, G12R, G12A, G13D, G13C, G13R, Q61H, Q61K, Q61L, Q61P, Q61R and Q61E. In some embodiments, the refractory cancer is associated with one or more mutations in NRAS selected from the group consisting of G12D, G12S, G12C, G12V, G12A, G13D, G13R, G13V, G13C, G13A, G13S, G61R, Q61K Q61H, and G61L. In some embodiments, the refractory cancer is associated with one or more mutations in HRAS selected from the group consisting of G12V, G12S, G12D, G12C, G12R, G12A, G13R, G13V, G13D, G13S, G13C, Q61R, Q61L, Q61K, and Q61H. In some embodiments, the refractory cancer is associated with one or more genetic alterations in BRAF selected from the group consisting of gene amplification, point mutation, BRAF fusion, and gene splicing events. In some embodiments, the refractory cancer is associated with one or more Class II or Class III mutations in BRAF. In some embodiments, the refractory cancer is associated with one or more mutations in BRAF selected from the group consisting of G464V, G469A, G469V, G469R, E586K, K601E, K601N, G466R, G466A, G466E, G466V, N581I, N581S, D594E, D594G, D594N, G596C, G596R, L597R, L597S, and L597Q. In some embodiments, the refractory cancer is associated with one or more alternative splicing events that result in the loss of BRAF gene exons 4-10, 4-8, 2-8 or 2-10. In still further embodiments of the foregoing, the method further comprises administering one or more pharmaceutical agents including anti-microtubular therapies, topoisomerase inhibitors, alkylating agents, nucleotide synthesis inhibitors, DNA synthesis inhibitors, protein synthesis inhibitors, developmental signaling pathway inhibitors, pro-apoptotic agents, RTK inhibitors (including inhibitors against ALK, EGFR, ERBB2, LTK, MET, NTRK, RET, ROS1), RAF inhibitors representing alternative binding modes (such as Type 1.5 or Type II), MEK1/2 inhibitors, ERK1/2 inhibitors, RSK1/2/3/4 inhibitors, AKT inhibitors, TORC1/2 inhibitors, DNA damage response pathway inhibitors (including ATM, ATR), PI3K inhibitors and/or radiation.

### Bcr-Abl Tyrosine Kinase Inhibition

In yet another aspect, provided herein is a method of inhibiting Bcr-Abl tyrosine kinase enzymatic activity, comprising contacting an effective amount of a compound or composition provided herein, to the Bcr-Abl tyrosine kinase. In some embodiments, provided herein is a method of inhibiting Bcr-Abl tyrosine kinase in a cell, comprising administering an effective amount of a compound or composition of the disclosure to the cell. In some embodiments, provided herein is a method of inhibiting Bcr-Abl tyrosine kinase in an individual in need thereof, comprising administering an effective amount of a compound or composition of the disclosure to the individual. In some variations, the compounds provided herein are selective for inhibiting Bcr-Abl tyrosine kinase. As such, in some embodiments, provided herein is a method of selectively inhibiting Bcr-Abl tyrosine kinase, as compared to other tyrosine kinases, including but not limited to c-KIT, FGFR, PDGFR, SRC, CSFR1, or VEGFR.

The compounds and compositions described herein may be used in a method of treating a disease or disorder mediated by Bcr-Abl tyrosine kinase activity. In some embodiments, the compound or composition is administered according to a dosage described herein.

In some embodiments, provided herein is a method for treating a disease or disorder mediated by Bcr-Abl tyrosine kinase activity comprising administering to an individual in need of treatment an effective amount of a compound of formula (I) or any variation thereof, or a pharmaceutically acceptable salt, solvate, hydrate, or co-crystal thereof, or a mixture of any of the foregoing. In some embodiments, the disease or disorder is a cancer mediated by Bcr-Abl tyrosine kinase activity. In some embodiments, the disease or disorder is chronic myeloid leukemia (CML), acute myeloid leukemia (AML), or acute lymphoblastic leukemia (ALL). In some embodiments, the disease or disorder is a cancer, such as leukemia. In some variations, the cancer is chronic myeloid leukemia (CMIL), Philadelphia-positive acute lymphoblastic leukemia (Ph+ ALL), acute myelogenous leukemia (AML), or mixed phenotype acute leukemia.

In certain embodiments, the leukemia is chronic myeloid leukemia. Chronic myeloid leukemia may be characterized by the state of disease progression, as determined by blast cells. In still further embodiments, the chronic myeloid leukemia is chronic phase CML, accelerated phase CML, or blastic phase CML. In some embodiments, the chronic myeloid leukemia is refractory chronic myeloid leukemia.

In some embodiments, the disease or disorder mediated by Bcr-Abl tyrosine kinase activity is refractory or resistant to first-line treatment, second-line treatment, and/or third-line treatment. In certain embodiments, the condition mediated by Bcr-Abl tyrosine kinase activity is refractory or resistant to treatment with one or more Bcr-Abl tyrosine kinase inhibitors selected from the group consisting of imatinib, nilotinib, dasatinib, bafetinib, bosutinib, radotinib, asciminib, and ponatinib. First-line treatment as described herein includes the use of imatinib; second- and third-line treatments as described herein include the use of nilotinib, dasatinib, bafetinib, bosutinib, radotinib, asciminib, and/or ponatinib. In some variations of the foregoing, the chronic myeloid leukemia is refractory chronic myeloid leukemia.

Resistant subtypes of Bcr-Abl tyrosine kinase-mediated diseases or disorders may be associated with any number of Bcr-Abl dependent or Bcr-Abl independent resistance mechanisms. In some embodiments wherein the disease or disorder mediated by Bcr-Abl tyrosine kinase activity is refractory to treatment, the disease or disorder is characterized as being associated with one or more Bcr-Abl dependent resistance mechanisms. Bcr-Abl dependent resistance mechanisms include, but are not limited to, one or more point mutations at positions M244, L248, G250, G250, Q252, Q252, Y253, Y253, E255, E255, D276, F311, T315, T315, F317, F317, M343, M351, E355, F359, F359, V379, F382, L387, H396, H396, S417, E459, F486, or T315 in the Bcr-Abl tyrosine kinase. In certain variations, the refractory disease or disorder mediated by Bcr-Abl tyrosine kinase is associated with one or more specific point mutations in the Bcr-Abl tyrosine kinase selected from the group consisting of: M244V, L248V, G250E, G250A, Q252H, Q252R, Y253F, Y253H, E255K, E255V, D276G, F311L, T315N, T315A, F317V, F317L, M343T, M351T, E355G, F359A, F359V, V379I, F382L, L387M, H396P, H396R, S417Y, E459K, F486S, and T315I. In certain embodiments, the refractory disease or disorder mediated by Bcr-Abl tyrosine kinase is associated with a T315I mutation. In still further embodiments, the refractory disease or disorder mediated by Bcr-Abl tyrosine kinase is associated with a T315I mutation at the onset of treatment and I315M mutation following ponatinib. In other embodiments, the refractory disease or disorder mediated by Bcr-Abl tyrosine kinase is associated with one or more P-loop mutations (M244V, G250E, Q252H, Y253H/F, E255K/V).

In some embodiments, provided is a method for treating cancer in an individual in need thereof, comprising administering to the individual an effective amount of a compound of formula (I), or any variation thereof as described herein. In some embodiments, the cancer is leukemia. In some embodiments, the cancer is chronic myeloid leukemia (CML), Philadelphia-positive acute lymphoblastic leukemia (Ph+ ALL), acute myelogenous leukemia (AML), or mixed phenotype acute leukemia. In some embodiments, the cancer is chronic myeloid leukemia (CML). In certain embodiments, the leukemia is chronic myeloid leukemia. In still further embodiments, the chronic myeloid leukemia is refractory chronic myeloid leukemia. In certain embodiments of the foregoing, the chronic myeloid leukemia is refractory chronic myeloid leukemia associated with a T315I mutation.

In one aspect, provided herein is a method of treating cancer in an individual in need thereof, wherein modulation of Bcr-Abl tyrosine kinase activity inhibits or ameliorates the pathology and/or symptomology of the cancer, comprising administering to the individual a therapeutically effective amount of a compound or composition provided herein. In one embodiment, provided herein is a method of treating cancer, wherein modulation of Bcr-Abl tyrosine kinase activity inhibits the pathology and/or symptomology of the cancer, in an individual, comprising administering to the individual a therapeutically effective amount of a compound or composition provided herein. In one embodiment, provided herein is a method of treating a cancer, wherein modulation of Bcr-Abl tyrosine kinase activity ameliorates the pathology and/or symptomology of the cancer, in an individual, comprising administering to the individual a therapeutically effective amount of a compound or composition provided herein.

In another aspect, provided herein is a method of preventing cancer, wherein modulation of Bcr-Abl tyrosine kinase activity prevents the pathology and/or symptomology of the cancer, in an individual, comprising administering to the individual a therapeutically effective amount of a compound or composition provided herein. In another aspect, provided herein is a method of delaying the onset and/or development of a cancer that is mediated by Bcr-Abl tyrosine kinase activity in an individual (such as a human) who is at risk for developing the cancer. It is appreciated that delayed development may encompass prevention in the event the individual does not develop the cancer.

In one aspect, provided herein is a method of delaying the onset and/or development of cancer in an individual in need thereof, comprising administering to the individual a therapeutically effective amount of a compound or composition provided herein. In some embodiments, the cancer is a leukemia. In certain embodiments, the cancer is chronic myeloid leukemia (CML), Philadelphia-positive acute lymphoblastic leukemia (Ph+ ALL), acute myelogenous leukemia (AML), or mixed phenotype acute leukemia. In some embodiments, the cancer is chronic myeloid leukemia. In still further embodiments, the chronic myeloid leukemia is refractory chronic myeloid leukemia. In still yet other embodiments, the chronic myeloid leukemia is refractory chronic myeloid leukemia associated with a T315I mutation. In one aspect, provided herein is a method of delaying the onset and/or development of chronic myeloid leukemia in an individual in need thereof, comprising administering to the individual a therapeutically effective amount of a compound or composition provided herein. In one variation, provided herein is a method of delaying the onset and/or development of refractory chronic myeloid in an individual in need thereof, comprising administering to the individual a therapeutically effective amount of a compound or composition provided herein. In one variation, provided herein is a method of delaying the onset and/or development of refractory chronic myeloid leukemia associated with a T315I mutation in an individual in need thereof, comprising administering to the individual a therapeutically effective amount of a compound or composition provided herein.

In one aspect, provided herein is a compound of formula (I) or any variation thereof, or a pharmaceutically acceptable salt, solvate, hydrate, or co-crystal thereof, or a mixture of any of the foregoing, for use in therapy. In some embodiments, provided herein is a compound of formula (I) or any variation thereof, or a pharmaceutically acceptable salt, solvate, hydrate, or co-crystal thereof, or a mixture of any of the foregoing, or pharmaceutical composition comprising such compound, for use in the treatment of cancer. In some embodiments, provided is a compound of formula (I) or any variation thereof, or a pharmaceutically acceptable salt, solvate, hydrate, or co-crystal thereof, or a mixture of any of the foregoing, or a pharmaceutical composition comprising such compound, for use in the treatment of chronic myeloid leukemia (CML), Philadelphia-positive acute lymphoblastic leukemia (Ph+ ALL), acute myelogenous leukemia (AML), or mixed phenotype acute leukemia. In some embodiments, provided is a compound of formula (I) or any variation thereof, or a pharmaceutically acceptable salt, solvate, hydrate, or co-crystal thereof, or a mixture of any of the foregoing, or a pharmaceutical composition comprising such compound, for use in the treatment of chronic myeloid leukemia (CML). In some embodiments, provided is a compound of formula (I) or any variation thereof, or a pharmaceutically acceptable salt, solvate, hydrate, or co-crystal thereof, or a mixture of any of the foregoing, or a pharmaceutical composition comprising such compound, for use in the treatment of refractory chronic myeloid leukemia (CMIL). In some embodiments, provided is a compound of formula (I) or any variation thereof, or a pharmaceutically acceptable salt, solvate, hydrate, or co-crystal thereof, or a mixture of any of the foregoing, or a pharmaceutical composition comprising such compound, for use in the treatment of refractory chronic myeloid leukemia associated with a T315I mutation.

In another embodiment, provided herein is a compound of formula (I) or any variation thereof, or a pharmaceutically acceptable salt, solvate, hydrate, or co-crystal thereof, or a mixture of any of the foregoing, for use in the manufacture of a medicament for the treatment of cancer. In another embodiment, provided herein is a compound of formula (I) or any variation thereof, or a pharmaceutically acceptable salt, solvate, hydrate, or co-crystal thereof, or a mixture of any of the foregoing, for use in the manufacture of a medicament for the treatment of chronic myeloid leukemia (CML), Philadelphia-positive acute lymphoblastic leukemia (Ph+ ALL), acute myelogenous leukemia (AML), or mixed phenotype acute leukemia. In some embodiments, the medicament is for the treatment of chronic myeloid leukemia. In some embodiments, the medicament is for the treatment of refractory chronic myeloid leukemia. In some embodiments, the medicament is for the treatment of refractory chronic myeloid leukemia associated with a T315I mutation.

In some embodiments, the individual is a mammal. In some embodiments, the individual is a primate, dog, cat, rabbit, or rodent. In some embodiments, the individual is a primate. In some embodiments, the individual is a human. In some embodiments, the human is at least about or is about any of 18, 21, 30, 50, 60, 65, 70, 75, 80, or 85 years old. In some embodiments, the human is a child. In some embodiments, the human is less than about or about any of 21, 18, 15, 10, 5, 4, 3, 2, or 1 years old.

In some embodiments, the method further comprises administering one or more additional pharmaceutical agents. In some embodiments, the method further comprises administering radiation. In some embodiments, the method further comprises administering one or more additional pharmaceutical agents, including anti-microtubular therapies (e.g. paclitaxel, vincristine), topoisomerase inhibitors (e.g. adriamycin), alylating agents (e.g. busulfan, cyclophosphamide), nucleotide synthesis inhibitors (hyroxyurea), DNA synthesis inhibtiors (e.g. cytarabine), protein synthesis inhibitors (e.g. omacetaxine), developmental signaling pathway inhibitors (e.g. sonidegib, Hedgehog pathway), pro-apoptotic agents (e.g. venetoclax), Abl myristoyl-pocket binding inhibitors (e.g. asciminib), MEK1/2 inhibitors (e.g. trametinib, binimetinib), AKT inhibitors (e.g. ipatasertib), PI3K inhibitors (e.g. apelisib) and radiation.

### VI. Dosing and Method of Administration

The dose of a compound described herein, or a pharmaceutically acceptable salt, solvate, hydrate, or co-crystal thereof, or a mixture of any of the foregoing, administered to an individual (such as a human) may vary with the particular compound or salt thereof, the method of administration, and the particular cancer, such as type and stage of cancer, being treated. In some embodiments, the amount of the compound, or a pharmaceutically acceptable salt, solvate, hydrate, or co-crystal thereof, or a mixture of any of the foregoing, is a therapeutically effective amount.

The compounds provided herein, or a pharmaceutically acceptable salt, solvate, hydrate, or co-crystal thereof, or a mixture of any of the foregoing, may be administered to an individual via various routes, including, e.g., intravenous, intramuscular, subcutaneous, oral, and transdermal.

The effective amount of the compound may in one aspect be a dose of between about 0.01 and about 100 mg/kg. Effective amounts or doses of the compounds of the present disclosure may be ascertained by routine methods, such as modeling, dose escalation, or clinical trials, taking into account routine factors, e.g., the mode or route of administration or drug delivery, the pharmacokinetics of the agent, the severity and course of the disease to be treated, the subject's health status, condition, and weight. An exemplary dose is in the range of about from about 0.7 mg to 7 g daily, or about 7 mg to 350 mg daily, or about 350 mg to 1.75 g daily, or about 1.75 to 7 g daily.

Any of the methods provided herein may in one aspect comprise administering to an individual a pharmaceutical composition that contains an effective amount of a compound provided herein, or a pharmaceutically acceptable salt, solvate, hydrate, or co-crystal thereof, or a mixture of any of the foregoing, and a pharmaceutically acceptable excipient.

A compound or composition provided herein may be administered to an individual in accordance with an effective dosing regimen for a desired period of time or duration, such as at least about one month, at least about 2 months, at least about 3 months, at least about 6 months, or at least about 12 months or longer, which in some variations may be for the duration of the individual's life. In one variation, the compound is administered on a daily or intermittent schedule. The compound can be administered to an individual continuously (for example, at least once daily) over a period of time. The dosing frequency can also be less than once daily, e.g., about a once weekly dosing. The dosing frequency can be more than once daily, e.g., twice or three times daily. The dosing frequency can also be intermittent, including a ‘drug holiday’ (e.g., once daily dosing for 7 days followed by no doses for 7 days, repeated for any 14 day time period, such as about 2 months, about 4 months, about 6 months or more). Any of the dosing frequencies can employ any of the compounds described herein together with any of the dosages described herein.

### VII. Articles of Manufacture and Kits

The present disclosure further provides articles of manufacture comprising a compound described herein, or a pharmaceutically acceptable salt, solvate, hydrate, or co-crystal thereof, or a mixture of any of the foregoing, a composition described herein, or one or more unit dosages described herein in suitable packaging. In certain embodiments, the article of manufacture is for use in any of the methods described herein. Suitable packaging is known in the art and includes, for example, vials, vessels, ampules, bottles, jars, flexible packaging and the like. An article of manufacture may further be sterilized and/or sealed.

The present disclosure further provides kits for carrying out the methods of the present disclosure, which comprises one or more compounds described herein or a composition comprising a compound described herein. The kits may employ any of the compounds disclosed herein. In one variation, the kit employs a compound described herein, or a pharmaceutically acceptable salt, solvate, hydrate, or co-crystal thereof, or a mixture of any of the foregoing, thereof. The kits may be used for any one or more of the uses described herein, and, accordingly, may contain instructions for the treatment of any disease or described herein, for example for the treatment of cancer or neoplastic disease, such as those associated with or mediated by RAF kinase or Bcr-Abl tyrosine kinase activity.

In some embodiments, the kit contains instructions for the treatment of a disease or disorder mediated by or associated with RAF kinase activity. In some embodiments, the disease or disorder is associated with one or more genetic alterations in KRAS, NRAS, HRAS, ARAF, BRAF or CRAF. In some embodiments, the kit contains instructions for the treatment of a disease or disorder mediated by or associated with Bcr-Abl tyrosine kinase activity, including chronic myeloid leukemia (CML), Philadelphia-positive acute lymphoblastic leukemia (Ph+ ALL), acute myelogenous leukemia (AML), or mixed phenotype acute leukemia. In some embodiments, the cancer is chronic myeloid leukemia. In some embodiments, the cancer is refractory chronic myeloid leukemia. In certain embodiments of the foregoing, the cancer is refractory chronic myeloid leukemia associated with a T315I mutation.

The kits optionally further comprise a container comprising one or more additional pharmaceutical agents and which kits further comprise instructions on or in the package insert for treating the subject with an effective amount of the one or more additional pharmaceutical agents.

Kits generally comprise suitable packaging. The kits may comprise one or more containers comprising any compound described herein. Each component (if there is more than one component) can be packaged in separate containers or some components can be combined in one container where cross-reactivity and shelf life permit.

The kits may be in unit dosage forms, bulk packages (e.g., multi-dose packages) or sub-unit doses. For example, kits may be provided that contain sufficient dosages of a compound as disclosed herein and/or an additional pharmaceutically active compound useful for a disease detailed herein to provide effective treatment of an individual for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of the compounds and instructions for use and be packaged in quantities sufficient for storage and use in pharmacies (e.g., hospital pharmacies and compounding pharmacies).

The kits may optionally include a set of instructions, generally written instructions, although electronic storage media (e.g., magnetic diskette or optical disk) containing instructions are also acceptable, relating to the use of component(s) of the methods of the present disclosure. The instructions included with the kit generally include information as to the components and their administration to an individual.

### EXAMPLES

It is understood that the present disclosure has been made only by way of example, and that numerous changes in the combination and arrangement of parts can be resorted to by those skilled in the art without departing from the spirit and scope of present disclosure.

### SYNTHETIC EXAMPLES

The chemical reactions in the Examples described can be readily adapted to prepare a number of other compounds disclosed herein, and alternative methods for preparing the compounds of this disclosure are deemed to be within the scope of this disclosure. For example, the synthesis of non-exemplified compounds according to the present disclosure can be successfully performed by modifications apparent to those skilled in the art, e.g., by appropriately protecting interfering groups, by utilizing other suitable reagents known in the art other than those described, or by making routine modifications of reaction conditions, reagents, and starting materials. Alternatively, other reactions disclosed herein or known in the art will be recognized as having applicability for preparing other compounds of the present disclosure.

Abbreviations used in the Examples include the following: ACN: acetonitrile; Brettphos: 2-(dicyclohexylphosphino)3,6-dimethoxy-2′,4′,6′-triisopropyl-1,1′-biphenyl; dppf: 1,1′-ferrocenediyl-bis(diphenylphosphine); DCM: dichloromethane; DMF: dimethylformamide; DMSO: dimethyl sulfoxide; EtOAc: ethyl acetate; EtOH: ethanol or ethyl alcohol; ¹H NMR: proton nuclear magnetic resonance; LCMS: liquid chromatography-mass spectrometry; LiHMDS: lithium hexamethyldisilazide; MeOH: methanol or methyl alcohol; NBS: N-bromosuccinimide; OAc: acetate; Py: pyridine; THF tetrahydrofuran; and TLC: thin-layer chromatography.

### Example 1. Synthesis of Intermediate B-1: 3-bromo-7-chloro-1-methyl-1,6-naphthyridin-2-one

### Step 1: Synthesis of ethyl (2E)-3-(4-amino-6-chloropyridin-3-yl)prop-2-enoate (B-c)

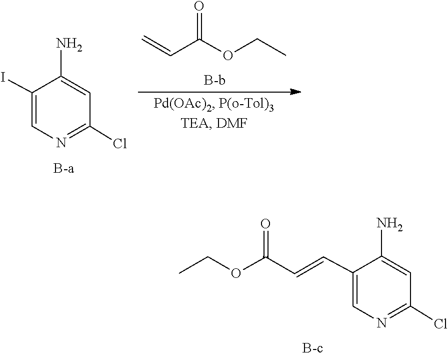

To a solution of 2-chloro-5-iodopyridin-4-amine (Compound B-a) (5.0 g, 19.65 mmol) in DMF (40.0 mL) was added ethyl acrylate (Compound B-b) (3.0 g, 29.47 mmol), P(o-Tol)₃ (478.5 mg, 1.57 mmol), TEA (2.8 g, 27.51 mmol) and Pd(OAc)₂ (176.5 mg, 0.79 mmol) at room temperature under N₂. The resulting mixture was stirred at 100° C. for 2 h under N₂. After the reaction was completed, the resulting mixture was diluted with H₂O and extracted with ethyl acetate. The combined organic layer was washed with brine, dried over anhydrous sodium sulfate, and filtered. The filtrate was concentrated under vacuum. The residue was purified by flash column chromatography with CH₂Cl₂/MeOH (97/3, v/v) to afford ethyl (2E)-3-(4-amino-6-chloropyridin-3-yl)prop-2-enoate (Compound B-c) (4.2 g, 94%) as a yellow solid. LCMS (ESI, m/z): [M+H]⁺=227.1.

### Step 2: Synthesis of 7-chloro-1H-1,6-naphthyridin-2-one (B-d)

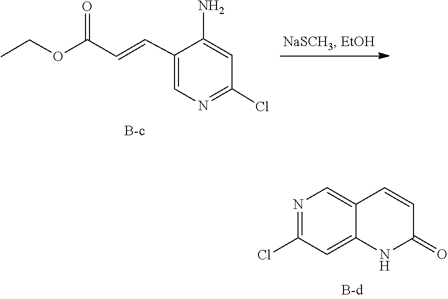

To a solution of ethyl (2E)-3-(4-amino-6-chloropyridin-3-yl)prop-2-enoate (Compound B-c) (4.2 g, 18.53 mmol) in ethanol (100.0 mL) was added sodium thiomethoxide (1.4 g, 20.38 mmol). The resulting mixture was stirred at 50° C. for 1 h. After the reaction was completed, the resulting mixture was concentrated under vacuum. The residue was purified by flash column chromatography with CH₂Cl₂/MeOH (8/1, v/v) to afford 7-chloro-1H-1,6-naphthyridin-2-one (Compound B-d) (2.4 g, 71%) as a yellow solid. LCMS (ESI, m/z): [M+H]⁺=181.0.

### Step 3: Synthesis of 3-bromo-7-chloro-1H-1,6-naphthyridin-2-one (B-e)

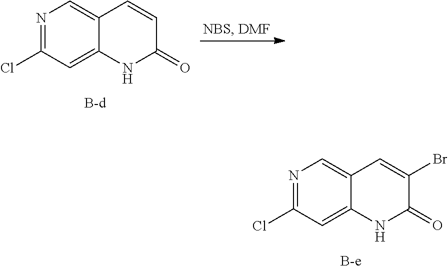 [SMILES: O=c1ccc2cnc(Cl)cc2[nH]1 ; O=c1[nH]c2cc(Cl)ncc2cc1Br]

To a solution of 7-chloro-1H-1,6-naphthyridin-2-one (Compound B-d) (1.0 g, 5.54 mmol) in DMF (15.0 mL) was added NBS (1.1 g, 6.09 mmol). The resulting mixture was stirred at 60° C. for 16 h. After the reaction was completed, the resulting mixture was diluted with H₂O and extracted with ethyl acetate. The combined organic layer was washed with brine, dried over anhydrous sodium sulfate, and filtered. The filtrate was concentrated under vacuum. The residue was purified by flash column chromatography with petroleum ether/ethyl acetate (60/40, v/v) to afford 3-bromo-7-chloro-1H-1,6-naphthyridin-2-one (Compound B-e) (1.0 g, 69%) as a yellow solid. LCMS (ESI, m/z): [M+H]⁺=258.9.

### Step 4: Synthesis of 3-bromo-7-chloro-1-methyl-1,6-naphthyridin-2-one (B-1)

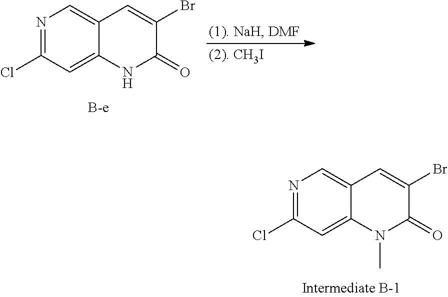

To a solution of 3-bromo-7-chloro-1H-1,6-naphthyridin-2-one (Compound B-e) (950.0 mg, 3.66 mmol) in DMF (10.0 mL) was added sodium hydride (292.9 mg, 7.32 mmol) at 0° C. under N₂. The mixture was stirred at 0° C. for 15 min. CH₃I (1.0 g, 7.32 mmol) was added to the mixture at 0° C. The mixture was stirred at room temperature for 1 h. After the reaction was completed, the resulting mixture was quenched by H₂O. The mixture was extracted with ethyl acetate. The combined organic layer was washed with brine, dried over anhydrous sodium sulfate, and filtered. The filtrate was concentrated under vacuum. The residue was washed with H₂O and filtered. The solid was collected and dried to afford 3-bromo-7-chloro-1-methyl-1,6-naphthyridin-2-one (Compound B-1) (1.0 g, crude) as a yellow solid. LCMS (ESI, m/z): [M+H]⁺=272.9.

### Example 2. Synthesis of Intermediate C-1: 7-chloro-1-methyl-3-(4-methylpyridin-3-yl)-1,6-naphthyridin-2-one

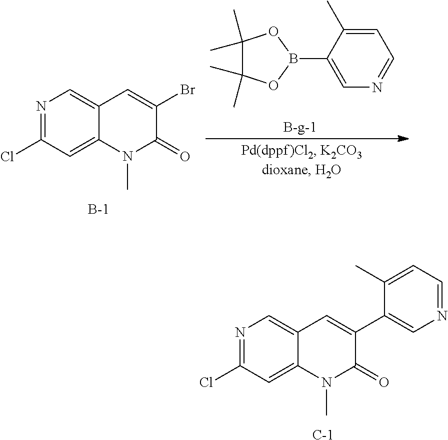

To a solution of 3-bromo-7-chloro-1-methyl-1,6-naphthyridin-2(1H)-one (Compound B-1) (400.0 mg, 1.46 mmol) in 1,4-dioxane/H₂O (10.0/2.0 mL) was added 4-methyl-3-(4,4,5,5-tetramethyl-1,3,2-dioxaborolan-2-yl)pyridine (Compound B-g-1) (384.5 mg, 1.76 mmol), K₂CO₃ (606.4 mg, 4.39 mmol) and Pd(dppf)Cl₂ (107.0 mg, 0.15 mmol) at room temperature under N₂. The resulting mixture was stirred at 80° C. for 2 h under N₂. After the reaction was completed, the resulting mixture was diluted with H₂O and extracted with ethyl acetate. The combined organic layer was washed with brine, dried over anhydrous sodium sulfate, and filtered. The filtrate was concentrated under vacuum. The residue was purified by flash column chromatography with petroleum ether/ethyl acetate (1/99, v/v) to afford 7-chloro-1-methyl-3-(4-methylpyridin-3-yl)-1,6-naphthyridin-2(1H)-one (Compound C-1) (280.0 mg, 67%) as a white solid. LCMS (ESI, m/z): [M+H]⁺=286.1.

### Example 3. Synthesis of Intermediate C-2: 7-chloro-3-(5-fluoro-4-methylpyridin-3-yl)-1-methyl-1,6-naphthyridin-2-one

### Step 1: Synthesis of 3-fluoro-4-methyl-5-(4,4,5,5-tetramethyl-1,3,2-dioxaborolan-2-yl)pyridine (B-g-2)

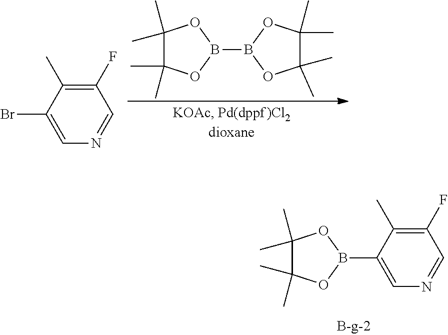

To a solution of 3-bromo-5-fluoro-4-methylpyridine (220.0 mg, 1.16 mmol) in 1,4-dioxane (6.0 mL) was added 4,4,4′,4′,5,5,5′,5′-octamethyl-2,2′-bi(1,3,2-dioxaborolane) (882.0 mg, 3.47 mmol), KOAc (340.9 mg, 3.47 mmol) and Pd(dppf)Cl₂ (84.7 mg, 0.12 mmol) at room temperature under N₂. The resulting mixture was stirred at 80° C. for 16 h under N₂. After the reaction was completed, the crude product was used directly without work up in the next step. LCMS (ESI, m/z): [M+H]⁺=238.1.

### Step 2: Synthesis of 7-chloro-3-(5-fluoro-4-methylpyridin-3-yl)-1-methyl-1,6-naphthyridin-2-one (C-2)

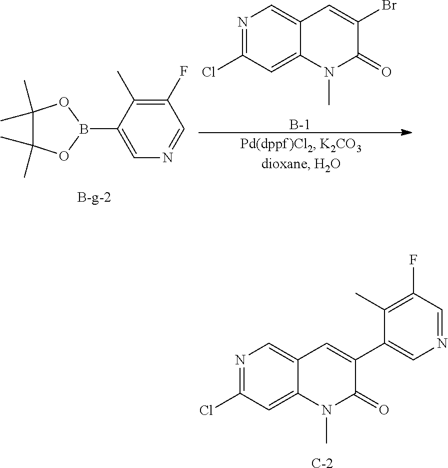

To a solution of 3-fluoro-4-methyl-5-(4,4,5,5-tetramethyl-1,3,2-dioxaborolan-2-yl)pyridine (Compound B-g-2) (400.0 mg, crude) in 1,4-dioxane (10.0 mL)/H₂O (1 mL) was added 3-bromo-7-chloro-1-methyl-1,6-naphthyridin-2-one (Compound B-1) (323.0 mg, 1.18 mmol), K₂CO₃ (489.7 mg, 3.54 mmol) and Pd(dppf)Cl₂ (86.4 mg, 0.12 mmol) at room temperature under N₂. The resulting mixture was stirred at 80° C. for 3 h under N₂. After the reaction was completed, the resulting mixture was concentrated under vacuum. The residue was purified by flash column chromatography with petroleum ether/ethyl acetate (70/30, v/v) to afford 7-chloro-3-(5-fluoro-4-methylpyridin-3-yl)-1-methyl-1,6-naphthyridin-2-one (Compound C-2) (110.0 mg, 30%) as a yellow solid. LCMS (ESI, m/z): [M+H]⁺=304.2.

### Example 4. Synthesis of Intermediate C-3: 7-chloro-1-methyl-3-(2-methylpyridin-3-yl)-1,6-naphthyridin-2-one

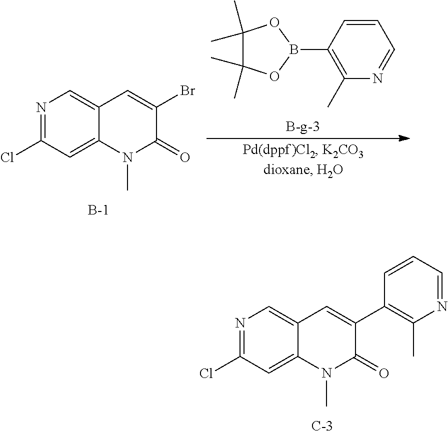

To a solution of 3-bromo-7-chloro-1-methyl-1,6-naphthyridin-2-one (Compound B-1) (200.0 mg, 0.73 mmol) in dioxane/H₂O (5.0/1.0 mL) was added 2-methyl-3-(4,4,5,5-tetramethyl-1,3,2-dioxaborolan-2-yl)pyridine (Compound B-g-3) (144.1 mg, 0.66 mmol), K₂CO₃ (303.1 mg, 2.19 mmol) and Pd(dppf)Cl₂ (53.5 mg, 0.07 mmol) at room temperature under N₂. The resulting mixture was stirred at 80° C. for 3 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (80/20, v/v) to afford 7-chloro-1-methyl-3-(2-methylpyridin-3-yl)-1,6-naphthyridin-2-one (Compound C-3) (180.0 mg, 86%) as a white solid. LCMS (ESI, m/z): [M+H]⁺=286.1.

### Example 5. Synthesis of Intermediate C-4: 7-chloro-3-(5-methoxy-4-methylpyridin-3-yl)-1-methyl-1,6-naphthyridin-2-one

### Step 1: Synthesis of 3-bromo-5-methoxy-4-methylpyridine

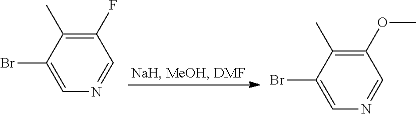

To a solution of 3-bromo-5-fluoro-4-methylpyridine (1.0 g, 5.26 mmol) in DMF (10.0 mL) was added MeOH (337.3 mg, 10.53 mmol) and sodium hydride (421.0 mg, 60% in oil) at room temperature under N₂. The resulting mixture was stirred at room temperature for 16 h under N₂. After the reaction was completed, the resulting mixture was quenched by water. The resulting mixture was filtered. The solid was washed with H₂O and dried to afford 3-bromo-5-methoxy-4-methylpyridine (660.0 mg, crude) as a yellow solid. LCMS (ESI, m/z): [M+H]⁺=202.1.

### Step 2: Synthesis of 3-methoxy-4-methyl-5-(4,4,5,5-tetramethyl-1,3,2-dioxaborolan-2-yl)pyridine (B-g-4)

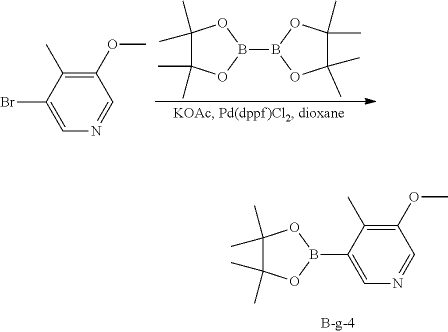

To a solution of 3-bromo-5-methoxy-4-methylpyridine (340.0 mg, 1.68 mmol) in 1,4-dioxane (10.0 mL) was added 4,4,4′,4′,5,5,5′,5′-octamethyl-2,2′-bi(1,3,2-dioxaborolane) (1.3 g, 5.05 mmol), KOAc (495.4 mg, 5.05 mmol) and Pd(dppf)Cl₂ (123.1 mg, 0.17 mmol) at room temperature under N₂. The resulting mixture was stirred at 80° C. for 16 h under N₂. After the reaction was completed, the crude product was used directly in the next step without purification. LCMS (ESI, m/z): [M+H]⁺=250.2.

### Step 3: Synthesis of 7-chloro-3-(5-methoxy-4-methylpyridin-3-yl)-1-methyl-1,6-naphthyridin-2-one (C-4)

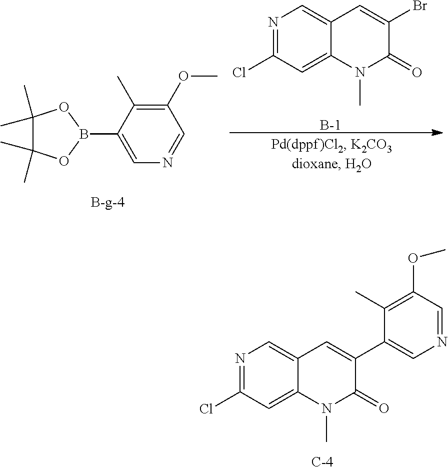

To a solution of 3-methoxy-4-methyl-5-(4,4,5,5-tetramethyl-1,3,2-dioxaborolan-2-yl)pyridine (Compound B-g-4) (500.0 mg, crude) in 1,4-dioxane (10.0 mL)/H₂O (1.0 mL) was added 3-bromo-7-chloro-1-methyl-1,6-naphthyridin-2-one (Compound B-1) (327.6 mg, 1.20 mmol), K₂CO₃ (496.6 mg, 3.59 mmol) and Pd(dppf)Cl₂ (87.6 mg, 0.12 mmol) at room temperature under N₂. The resulting mixture was stirred at 80° C. for 3 h under N₂. After the reaction was completed, the resulting mixture was concentrated under vacuum. The residue was purified by flash column chromatography with petroleum ether/ethyl acetate (70/30, v/v) to afford 7-chloro-3-(5-methoxy-4-methylpyridin-3-yl)-1-methyl-1,6-naphthyridin-2-one (Compound C-4) (70.0 mg, 18%) as a yellow solid. LCMS (ESI, m/z): [M+H]⁺=316.1.

### Example 6. Synthesis of Intermediate C-5: 7-chloro-3-(5-hydroxy-4-methylpyridin-3-yl)-1-methyl-1,6-naphthyridin-2-one

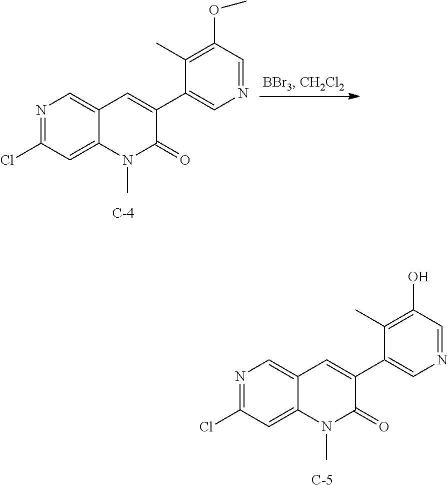

To a solution of 7-chloro-3-(5-methoxy-4-methylpyridin-3-yl)-1-methyl-1,6-naphthyridin-2-one (Compound C-4) (430.0 mg, 1.36 mmol) in CH₂Cl₂ (5.0 mL) was added BBr₃/DCM (1.29 mL). The resulting mixture was stirred at room temperature for 16 h. After the reaction was completed, the resulting mixture was concentrated under vacuum. The residue was purified by flash column chromatography with petroleum ether/ethyl acetate (70/30, v/v) to afford 7-chloro-3-(5-hydroxy-4-methylpyridin-3-yl)-1-methyl-1,6-naphthyridin-2-one (Compound C-5) (90.0 mg, 22%) as a yellow solid. LCMS (ESI, m/z): [M+H]⁺=302.1.

### Example 7. Synthesis of Intermediate C-6: 7-chloro-3-(2,4-dimethylpyridin-3-yl)-1-methyl-1,6-naphthyridin-2-one

### Step 1: Synthesis of 2,4-dimethyl-3-(4,4,5,5-tetramethyl-1,3,2-dioxaborolan-2-yl)pyridine (B-g-6)

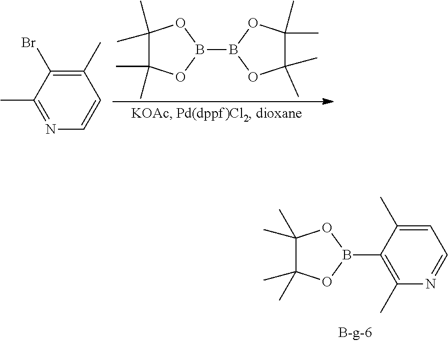

To a solution of 3-bromo-2,4-dimethylpyridine (300.0 mg, 1.61 mmol) in 1,4-dioxane (10.0 mL) was added bis(pinacolato)diboron (1.2 g, 4.84 mmol), KOAc (474.7 mg, 4.84 mmol) and Pd(dppf)Cl₂ (118.0 mg, 0.16 mmol). The resulting mixture was stirred at room temperature for 16 h. After the reaction was completed, the resulting mixture was concentrated under vacuum. The residue was purified by flash column chromatography with petroleum ether/ethyl acetate (5/1, v/v) to afford 2,4-dimethyl-3-(4,4,5,5-tetramethyl-1,3,2-dioxaborolan-2-yl)pyridine (Compound B-g-6) (250.0 mg, 67%) as a yellow solid. LCMS (ESI, m/z): [M+H]⁺=234.2.

### Step 2: Synthesis of 7-chloro-3-(2,4-dimethylpyridin-3-yl)-1-methyl-1,6-naphthyridin-2-one (C-6)

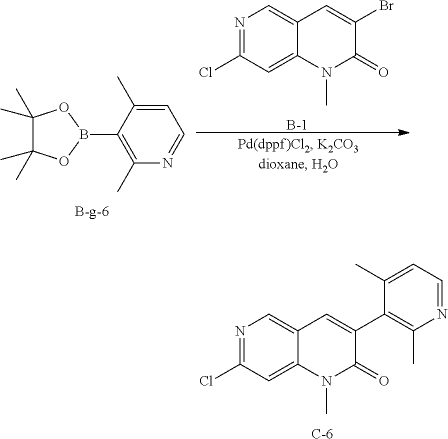

To a stirred solution of 2,4-dimethyl-3-(4,4,5,5-tetramethyl-1,3,2-dioxaborolan-2-yl)pyridine (Compound B-g-6) (250.0 mg, 1.07 mmol) in 1,4-dioxane/H₂O (5.0/1.0 mL) was added 3-bromo-7-chloro-1-methyl-1,6-naphthyridin-2-one (Compound B-1) (293.3 mg, 1.07 mmol), K₂CO₃ (444.6 mg, 3.22 mmol) and Pd(dppf)Cl₂ (78.5 mg, 0.11 mmol). The resulting mixture was stirred at 100° C. for 2 h under N₂. After the reaction was completed, the resulting mixture was concentrated under vacuum. The residue was purified by flash column chromatography with CH₂Cl₂/MeOH (94/6, v/v) to afford 7-chloro-3-(2,4-dimethylpyridin-3-yl)-1-methyl-1,6-naphthyridin-2-one (Compound C-6) (120.0 mg, 49%) as a yellow solid. LCMS (ESI, m/z): [M+H]⁺=300.1.

### Example 8. Synthesis of Intermediate C-7: 7-chloro-3-(2,6-dimethylpyridin-3-yl)-1-methyl-1,6-naphthyridin-2-one

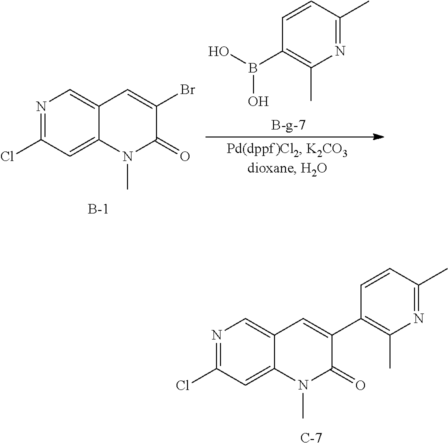

To a solution of 3-bromo-7-chloro-1-methyl-1,6-naphthyridin-2-one (Compound B-1) (200.0 mg, 0.73 mmol) in 1,4-dioxane/H₂O (5.0/1.0 mL) was added 2,6-dimethylpyridin-3-ylboronic acid (Compound B-g-7) (132.5 mg, 0.88 mmol), K₂CO₃ (303.2 mg, 2.19 mmol) and Pd(dppf)Cl₂ (53.5 mg, 0.07 mmol) at room temperature under N₂. The resulting mixture was stirred at 100° C. for 3 h under N₂. After the reaction was completed, the resulting mixture was diluted with H₂O and extracted with ethyl acetate. The combined organic layer was washed with brine, dried over anhydrous sodium sulfate, and filtered. The filtrate was concentrated under reduced pressure. The residue was purified by flash column chromatography with petroleum ether/ethyl acetate (50/50, v/v) to afford 7-chloro-3-(2,6-dimethylpyridin-3-yl)-1-methyl-1,6-naphthyridin-2-one (Compound C-7) (120.0 mg, 55%) as a white solid. LCMS (ESI, m/z): [M+H]⁺=300.1.

### Example 9. Synthesis of Intermediate C-8: 7-chloro-3-(4,6-dimethylpyridin-3-yl)-1-methyl-1,6-naphthyridin-2(1H)-one

### Step 1: Synthesis of 2,4-dimethyl-5-(4,4,5,5-tetramethyl-1,3,2-dioxaborolan-2-yl)pyridine B-g-8)

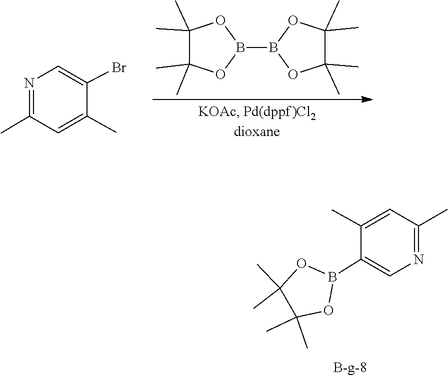

To a solution of 5-bromo-2,4-dimethylpyridine (300.0 mg, 1.61 mmol) in 1,4-dioxane (30.0 mL) was added 4,4,4′,4′,5,5,5′,5′-octamethyl-2,2′-bi(1,3,2-dioxaborolane) (1228.4 mg, 4.84 mmol), KOAc (474.8 mg, 4.84 mmol) and Pd(dppf)Cl₂ (118.0 mg, 0.16 mmol) at room temperature under N₂. The resulting mixture was stirred at 85° C. for 16 h under N₂. After the reaction was completed, the crude mixture was used directly in the next step. LCMS (ESI, m/z): [M+H]⁺=234.2.

### Step 2: Synthesis of 7-chloro-3-(4,6-dimethylpyridin-3-yl)-1-methyl-1,6-naphthyridin-2(1H)-one (C-8)

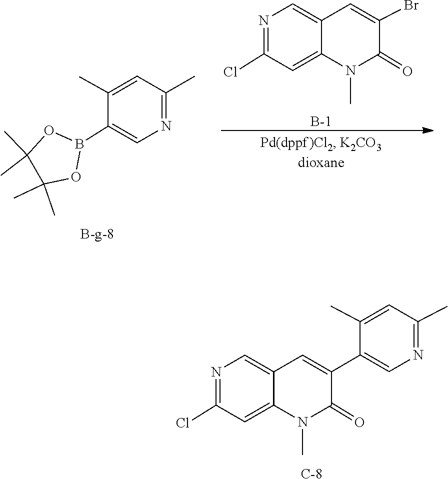

To a solution of 2,4-dimethyl-5-(4,4,5,5-tetramethyl-1,3,2-dioxaborolan-2-yl)pyridine (Compound B-g-8) (600.0 mg, crude) in 1,4-dioxane/H₂O (30.0/6.0 mL) was added 3-bromo-7-chloro-1-methyl-1,6-naphthyridin-2(1H)-one (434.8 mg, 1.59 mmol), K₂CO₃ (1098.5 mg, 7.95 mmol) and Pd(dppf)Cl₂ (116.3 mg, 0.16 mmol) at room temperature under N₂. The resulting mixture was stirred at 70° C. for 2 h under N₂. After the reaction was completed, the resulting mixture was diluted with H₂O and extracted with ethyl acetate. The combined organic layer was washed with brine, dried over anhydrous sodium sulfate, and filtered. The filtrate was concentrated under vacuum. The residue was purified by flash column chromatography with petroleum ether/ethyl acetate (1/99, v/v) to afford 7-chloro-3-(4,6-dimethylpyridin-3-yl)-1-methyl-1,6-naphthyridin-2(1H)-one (Compound C-8) (290.0 mg, 61%) as a brown solid. LCMS (ESI, m/z): [M+H]⁺=300.1.

### Example 10. Synthesis of Intermediate D1: (1R,2R)-2-fluorocyclopropane-1-carboxamide

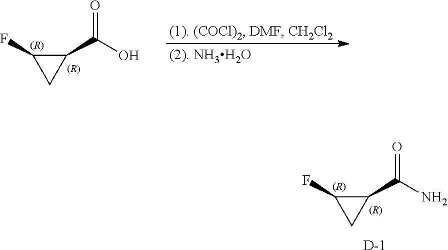

To a solution of (1R,2R)-2-fluorocyclopropane-1-carboxylic acid (200.0 mg, 1.92 mmol) in CH₂Cl₂ (5.0 mL) was added DMF (0.1 mL) and oxalyl dichloride (292.7 mg, 2.31 mmol) at room temperature. The resulting mixture was stirred at room temperature for 1 h. After the reaction was completed, the resulting mixture was poured into NH₃/CH₃OH (5.0 mL, 4 mol/L) at room temperature. The resulting mixture was stirred at room temperature for another 10 min. After the reaction was completed, the resulting mixture was concentrated under vacuum to afford (1R,2R)-2-fluorocyclopropane-1-carboxamide (Compound D-1) (280.0 mg, crude) as a white solid. LCMS (ESI, m/z): [M+H]⁺=104.0.

### Example 11. Synthesis of Intermediate D2: (1S,2S)-2-fluorocyclopropane-1-carboxamide

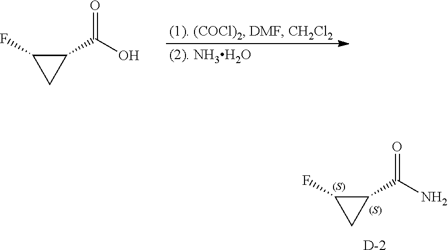

To a solution of (1S,2S)-2-fluorocyclopropane-1-carboxylic acid (200.0 mg, 1.92 mmol) in CH₂Cl₂ (5.0 mL) was added DMF (0.1 mL) and oxalyl dichloride (292.7 mg, 2.31 mmol) at room temperature. The resulting mixture was stirred at room temperature for 1 h. After the reaction was completed, the resulting mixture was poured into NH₃/CH₃OH (5.0 mL, 4 mol/L) at room temperature. The resulting mixture was stirred at room temperature for another 10 min. After the reaction was completed, the resulting mixture was concentrated under vacuum to afford (1S,2S)-2-fluorocyclopropane-1-carboxamide (Compound D-2) (250.0 mg, crude) as a white solid. LCMS (ESI, m/z): [M+H]⁺=104.0.

### Example 12. Synthesis of Intermediate D3: (R)-2,2-difluorocyclopropane-1-carboxamide

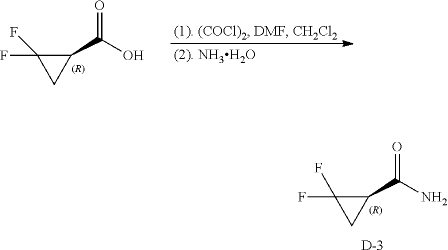

To a solution of (R)-2,2-difluorocyclopropane-1-carboxylic acid (100.0 mg, 0.82 mmol) in CH₂Cl₂ (5.0 mL) was added DMF (0.01 mL) and oxalyl dichloride (104.0 mg, 0.82 mmol) at room temperature. The resulting mixture was stirred at room temperature for 1 h. Then the resulting mixture was added into NH₃/CH₃OH (5.0 mL, 7 mol/L) at room temperature. The resulting mixture was stirred at room temperature for another 10 min. After the reaction was completed, the resulting mixture was concentrated under vacuum to afford (R)-2,2-difluorocyclopropane-1-carboxamide (Compound D-3) (180.0 mg, crude) as a white solid. LCMS (ESI, m/z): [M+H]⁺=122.0.

### Example 13. Synthesis of Intermediate C-9: 7-chloro-3-(6-(hydroxymethyl)-4-methylpyridin-3-yl)-1-methyl-1,6-naphthyridin-2(1H)-one

### Step 1: Synthesis of methyl 4-methyl-5-(4,4,5,5-tetramethyl-1,3,2-dioxaborolan-2-yl)picolinate (Compound B-g′-9)

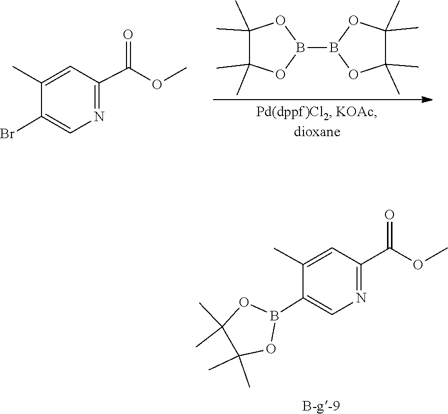

To a solution of methyl 5-bromo-4-methylpicolinate (2.7 g, 11.74 mmol) in dioxane (30.0 mL) was added 4,4,4′,4′,5,5,5′,5′-octamethyl-2,2′-bi(1,3,2-dioxaborolane) (8.9 g, 35.21 mmol), KOAc (3.5 g, 35.21 mmol) and Pd(dppf)Cl₂ (0.9 g, 1.17 mmol) at room temperature under N₂. The resulting mixture was stirred at 80° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (3/2, v/v) to afford methyl 4-methyl-5-(4,4,5,5-tetramethyl-1,3,2-dioxaborolan-2-yl)picolinate (Compound B-g′-9) (1.8 g, 55%) as a brown oil. LCMS (ESI, m/z): [M+H]⁺=278.1.

### Step 2: Synthesis of methyl 5-(7-chloro-1-methyl-2-oxo-1,2-dihydro-1,6-naphthyridin-3-yl)-4-methylpicolinate (Compound C′-9)

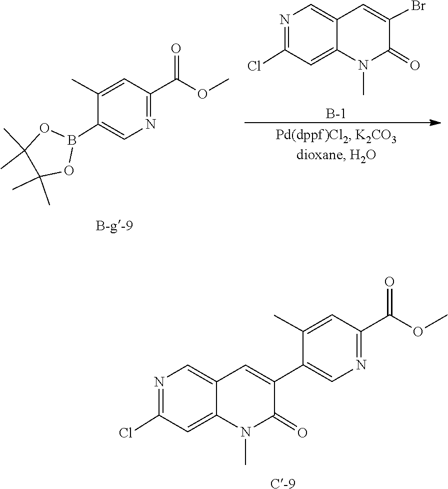

To a solution of methyl 4-methyl-5-(4,4,5,5-tetramethyl-1,3,2-dioxaborolan-2-yl)picolinate (Compound B-g′-9) (800.0 mg, 2.89 mmol) in dioxane/H₂O (20.0/5.0 mL) was added 3-bromo-7-chloro-1-methyl-1,6-naphthyridin-2(1H)-one (Compound B-1) (789.6 mg, 2.89 mmol), K₂CO₃ (1994.8 mg, 14.43 mmol) and Pd(dppf)Cl₂ (211.2 mg, 0.29 mmol) at room temperature under N₂. The resulting mixture was stirred at 60° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/MeOH (10/1, v/v) to afford methyl 5-(7-chloro-1-methyl-2-oxo-1,2-dihydro-1,6-naphthyridin-3-yl)-4-methylpicolinate (Compound C′-9) (0.4 g, 40%) as a white solid. LCMS (ESI, m/z): [M+H]⁺=344.1.

### Step 3: Synthesis of 7-chloro-3-(6-(hydroxymethyl)-4-methylpyridin-3-yl)-1-methyl-1,6-naphthyridin-2(1H)-one (Compound C-9)

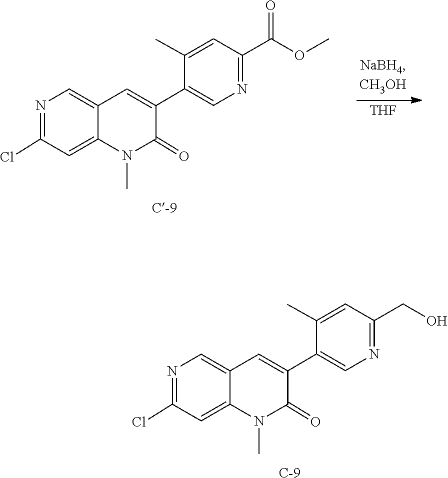

To a solution of methyl 5-(7-chloro-1-methyl-2-oxo-1,2-dihydro-1,6-naphthyridin-3-yl)-4-methylpicolinate (Compound C′-9) (300.0 mg, 0.87 mmol) in THF/CH₃OH (5.0/5.0 mL) was added NaBH₄ (99.1 mg, 2.62 mmol) at room temperature. The resulting mixture was stirred at 80° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/MeOH (10/1, v/v) to afford 7-chloro-3-(6-(hydroxymethyl)-4-methylpyridin-3-yl)-1-methyl-1,6-naphthyridin-2(1H)-one (Compound C-9) (53.0 mg, 19%) as a colorless oil. LCMS (ESI, m/z): [M+H]⁺=316.1.

### Example 14. Synthesis of Intermediate C-10: 7-chloro-3-(5-fluoro-4-methylpyridin-3-yl)-1H-1,6-naphthyridin-2-one

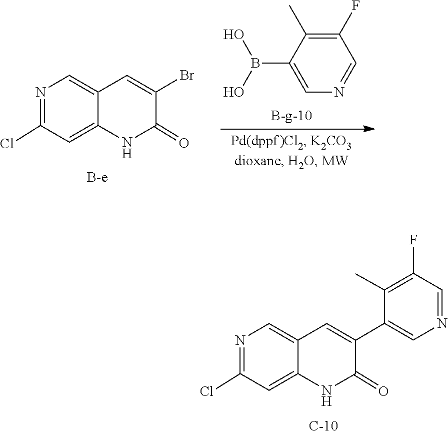

To a solution of 3-bromo-7-chloro-1H-1,6-naphthyridin-2-one (Compound B-e) (200.0 mg, 0.77 mmol) in 1,4-dioxane/H₂O (5.0/1.0 mL) was added 5-fluoro-4-methylpyridin-3-ylboronic acid (Compound B-g-10) (119.4 mg, 0.77 mmol), K₂CO₃ (319.6 mg, 2.31 mmol) and Pd(dppf)Cl₂ (125.9 mg, 0.15 mmol). The resulting mixture was stirred with microwave radiation at 120° C. for 1.5 h under N₂. After the reaction was completed, the resulting mixture was concentrated under vacuum. The residue was purified by flash column chromatography with petroleum ether/EtOAc (30/70, v/v) to afford 7-chloro-3-(5-fluoro-4-methylpyridin-3-yl)-1H-1,6-naphthyridin-2-one (Compound C-10) (100.0 mg, 49%) as a yellow solid. LCMS (ESI, m/z): [M+H]⁺=290.0.

### Example 15. Synthesis of Intermediate C-11: 2-[7-chloro-3-(5-fluoro-4-methylpyridin-3-yl)-2-oxo-1,6-naphthyridin-1-yl]acetonitrile

### Step 1: Synthesis of 2-(3-bromo-7-chloro-2-oxo-1,6-naphthyridin-1-yl)acetonitrile (B-2)

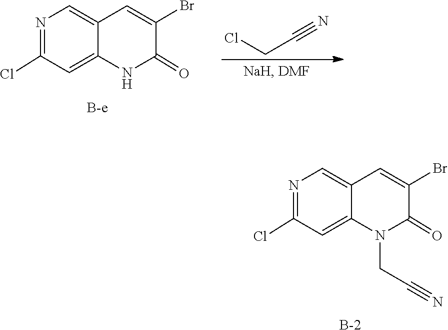

To a solution of 3-bromo-7-chloro-1H-1,6-naphthyridin-2-one (Compound B-e) (500.0 mg, 1.92 mmol) in DMF (5.0 mL) was added NaH (55.5 mg, 2.31 mmol) at 0° C. under N₂. The resulting mixture was stirred at 0° C. for 1 h. Then chloroacetonitrile (174.5 mg, 2.31 mmol) was added to the mixture at 0° C. under N₂. The resulting mixture was stirred at 0° C. for another 3 h. After the reaction was completed, the reaction was quenched with H₂O at 0° C. and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (4/1, v/v) to afford 2-(3-bromo-7-chloro-2-oxo-1,6-naphthyridin-1-yl)acetonitrile (Compound B-2) (250.0 mg, 43%) as a white solid. LCMS (ESI, m/z): [M+H]⁺=297.9.

### Step 2: Synthesis of 2-[7-chloro-3-(5-fluoro-4-methylpyridin-3-yl)-2-oxo-1,6-naphthyridin-1-yl]acetonitrile (C-11)

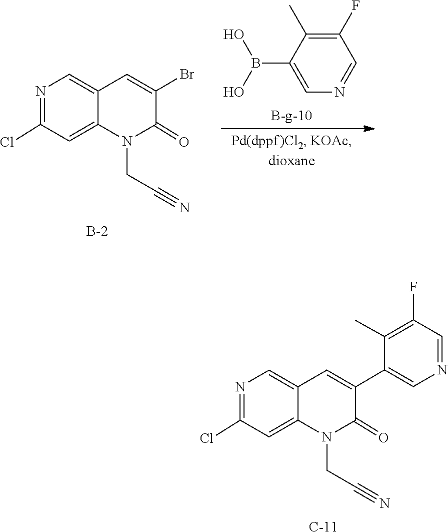

To a solution of 2-(3-bromo-7-chloro-2-oxo-1,6-naphthyridin-1-yl)acetonitrile (Compound B-2) (230.0 mg, 0.77 mmol) in 1,4-dioxane (5.0 mL) was added 5-fluoro-4-methylpyridin-3-ylboronic acid (Compound B-g-10) (119.4 mg, 0.77 mmol), KOAc (226.8 mg, 2.31 mmol) and Pd(dppf)Cl₂ (125.3 mg, 0.16 mmol) at room temperature under N₂. The resulting mixture was stirred at 80° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (40/60, v/v) to afford 2-[7-chloro-3-(5-fluoro-4-methylpyridin-3-yl)-2-oxo-1,6-naphthyridin-1-yl]acetonitrile (Compound C-11) (140.0 mg, 11%) as a white solid. LCMS (ESI, m/z): [M+H]⁺=329.1.

### Example 16. Synthesis of Intermediate C′-12: 3-(6-(1-((tert-butyldimethylsilyl)oxy)-2,2,2-trifluoroethyl)-4-methylpyridin-3-yl)-7-chloro-1-methyl-1,6-naphthyridin-2(1H)-one

### Step 1: Synthesis of 1-(5-bromo-4-methylpyridin-2-yl)-2,2,2-trifluoroethan-1-ol

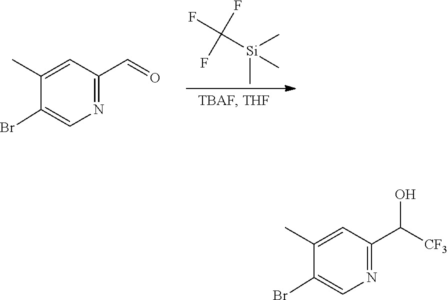

To a mixture of 5-bromo-4-methylpyridine-2-carbaldehyde (1.0 g, 5.00 mmol) and trimethyl(trifluoromethyl)silane (1.4 g, 10.00 mmol) in THE (15.0 mL) was added dropwise TBAF (15.0 mL, 1 mol/L) at 0° C. under N₂. The resulting mixture was stirred at 0° C. for 16 h. After the reaction was completed, the reaction was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (92/8, v/v) to afford 1-(5-bromo-4-methylpyridin-2-yl)-2,2,2-trifluoroethan-1-ol (1.0 g, 74%) as a white solid. LCMS (ESI, m/z): [M+H]⁺=270.0.

### Step 2: Synthesis of 5-bromo-2-(1-((tert-butyldimethylsilyl)oxy)-2,2,2-trifluoroethyl)-4-methylpyridine

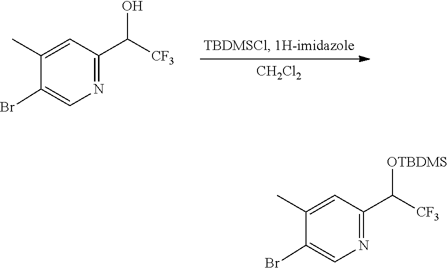 [SMILES: Cc1cc(C(O)C(F)(F)F)ncc1Br ; "CH2Cl2" ; Cc1cc(C(O[Si](C)(C)C(C)(C)C)C(F)(F)F)ncc1Br]

To a solution of 1-(5-bromo-4-methylpyridin-2-yl)-2,2,2-trifluoroethan-1-ol (1.0 g, 3.70 mmol) in CH₂Cl₂ (20.0 mL) was added TBDMSCl (1.1 g, 7.41 mmol), 1H-imidazole (554.6 mg, 8.15 mmol) and DMAP (90.5 mg, 0.74 mmol) at room temperature. The resulting mixture was stirred at room temperature for 16 h. After the reaction was completed, the resulting mixture was concentrated under reduced pressure. The residue was purified by flash column chromatography with petroleum ether/ethyl acetate (94/6, v/v) to afford 5-bromo-2-(1-((tert-butyldimethylsilyl)oxy)-2,2,2-trifluoroethyl)-4-methylpyridine (1.1 g, 74%) as a colorless oil. LCMS (ESI, m/z): [M+H]⁺=384.1.

### Step 3: Synthesis of 2-(1-((tert-butyldimethylsilyl)oxy)-2,2,2-trifluoroethyl)-4-methyl-5-(4,4,5,5-tetramethyl-1,3,2-dioxaborolan-2-yl)pyridine (B-g′-12)

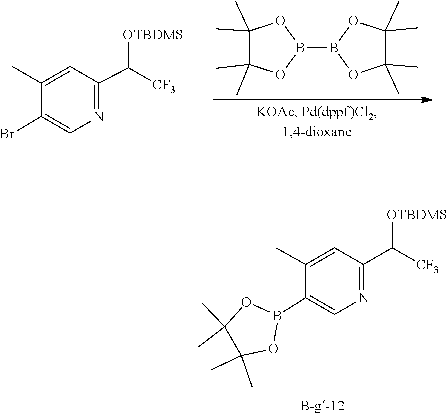

To a solution of 5-bromo-2-(1-((tert-butyldimethylsilyl)oxy)-2,2,2-trifluoroethyl)-4-methylpyridine (1.1 g, 2.76 mmol) in 1,4-dioxane (15.0 mL) was added 4,4,4′,4′,5,5,5′,5′-octamethyl-2,2′-bi(1,3,2-dioxaborolane) (2.1 g, 8.28 mmol), KOAc (812.1 mg, 8.28 mmol) and Pd(dppf)Cl₂ (201.8 mg, 0.28 mmol) at room temperature under N₂. The resulting mixture was stirred at 80° C. for 16 h. After the reaction was completed, the resulting mixture was concentrated under reduced pressure to afford 2-(1-((tert-butyldimethylsilyl)oxy)-2,2,2-trifluoroethyl)-4-methyl-5-(4,4,5,5-tetramethyl-1,3,2-dioxaborolan-2-yl)pyridine (Compound B-g′-12) (2.4 g, crude) as a brown solid. LCMS (ESI, m/z): [M+H]⁺=432.2.

### Step 4: Synthesis of 3-(6-(1-((tert-butyldimethylsilyl)oxy)-2,2,2-trifluoroethyl)-4-methylpyridin-3-yl)-7-chloro-1-methyl-1,6-naphthyridin-2(1H)-one (C′-12)

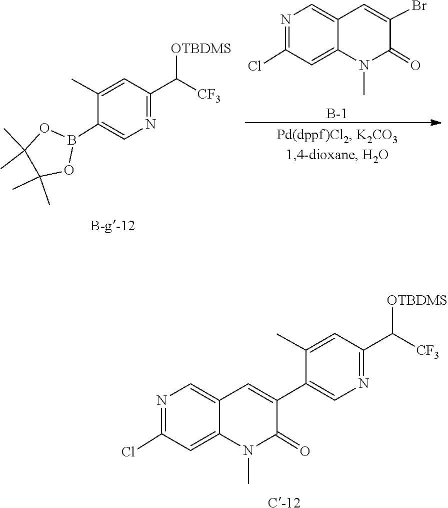

To a solution of 2-(1-((tert-butyldimethylsilyl)oxy)-2,2,2-trifluoroethyl)-4-methyl-5-(4,4,5,5-tetramethyl-1,3,2-dioxaborolan-2-yl)pyridine (Compound B-g′-12) (2.4 g, 2.75 mmol) in 1,4-dioxane/H₂O (15.0/3.0 mL) was added 3-bromo-7-chloro-1-methyl-1,6-naphthyridin-2(1H)-one (Compound B-1) (751.9 mg, 2.75 mmol), K₂CO₃ (1.9 g, 13.75 mmol) and Pd(dppf)Cl₂ (201.8 mg, 0.28 mmol) at room temperature under N₂. The resulting mixture was stirred at 80° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (65/35, v/v) to afford 3-(6-(1-((tert-butyldimethylsilyl)oxy)-2,2,2-trifluoroethyl)-4-methylpyridin-3-yl)-7-chloro-1-methyl-1,6-naphthyridin-2(1H)-one (Compound C′-12) (870.0 mg, 31%) as a yellow oil. LCMS (ESI, m/z): [M+H]⁺=498.2.

### Example 17. Synthesis of Intermediate C-13: 7-chloro-3-(4-methylpyridin-3-yl)-1H-1,6-naphthyridin-2-one

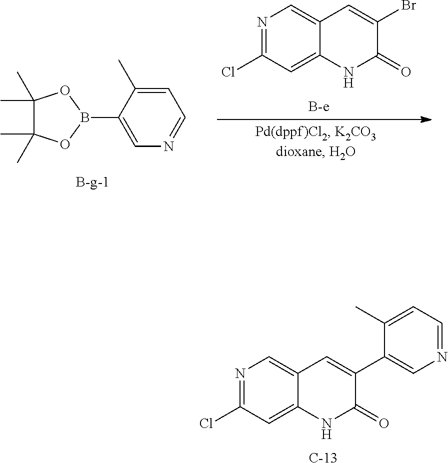

To a solution of 3-bromo-7-chloro-1H-1,6-naphthyridin-2-one (Compound B-e) (200.0 mg, 0.77 mmol) in 1,4-dioxane/H₂O (10.0/2.0 mL) was added 4-methyl-3-(4,4,5,5-tetramethyl-1,3,2-dioxaborolan-2-yl)pyridine (Compound B-g-1) (202.6 mg, 0.93 mmol), K₂CO₃ (319.6 mg, 2.31 mmol) and Pd(dppf)Cl₂ (56.4 mg, 0.08 mmol). The final reaction mixture was irradiated with microwave radiation at 120° C. for 1.5 h. After the reaction was completed, the resulting mixture was concentrated under vacuum. The residue was purified by flash column chromatography with petroleum ether/EtOAc (70/30, v/v) to afford 7-chloro-3-(4-methylpyridin-3-yl)-1H-1,6-naphthyridin-2-one (Compound C-13) (150.0 mg, 72%) as a yellow solid. LCMS (ESI, m/z): [M+H]⁺=272.1.

### Example 18. Synthesis of Intermediate C-14: 7-chloro-3-[6-[(1R)-1-hydroxyethyl]-4-methylpyridin-3-yl]-1-methyl-1,6-naphthyridin-2-one

### Step 1: Synthesis of (R)-1-[4-methyl-5-(4,4,5,5-tetramethyl-1,3,2-dioxaborolan-2-yl)pyridin-2-yl]ethanol (B-X-g)

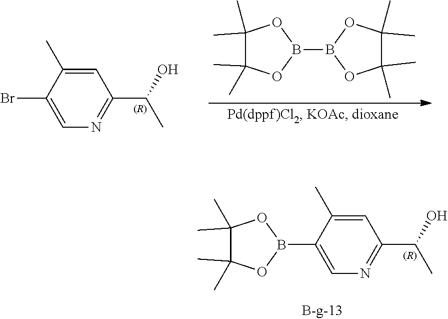

A mixture of (1R)-1-(5-bromo-4-methylpyridin-2-yl)ethanol (1.0 g, 4.63 mmol), bis(pinacolato)diboron (1.76 g, 6.94 mmol), KOAc (1.4 g, 13.88 mmol) and Pd(dppf)Cl₂ (0.3 g, 0.46 mmol) in dioxane (30.0 mL) was stirred at 100° C. for 16 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (94/6, v/v) to afford (1R)-1-[4-methyl-5-(4,4,5,5-tetramethyl-1,3,2-dioxaborolan-2-yl)pyridin-2-yl]ethanol (Compound B-g-13) (500.0 mg, 41%) as a yellow solid. LCMS (ESI, m/z): [M+H]⁺=264.2.

### Step 2: Synthesis of 7-chloro-3-[6-[(1R)-1-hydroxyethyl]-4-methylpyridin-3-yl]-1-methyl-1,6-naphthyridin-2-one (C-14)

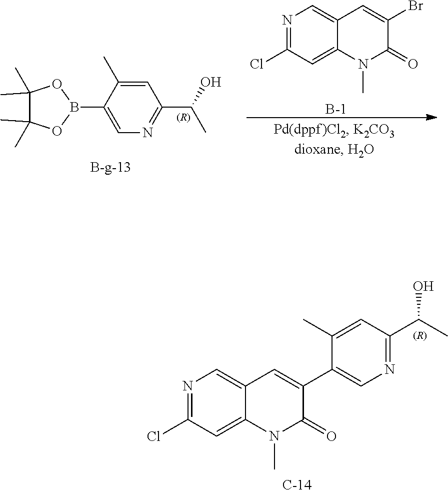

A mixture of (1R)-1-[4-methyl-5-(4,4,5,5-tetramethyl-1,3,2-dioxaborolan-2-yl)pyridin-2-yl]ethanol (Compound B-g-13) (500.0 mg, 1.90 mmol), 3-bromo-7-chloro-1-methyl-1,6-naphthyridin-2-one (Compound B-1) (519.7 mg, 1.90 mmol), K₂CO₃ (787.8 mg, 5.70 mmol) and Pd(dppf)Cl₂ (139.0 mg, 0.19 mmol) in dioxane (10.0 mL) and H₂O (1.0 mL) was stirred at 80° C. for 16 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (93/7, v/v) to afford 7-chloro-3-[6-[(1R)-1-hydroxyethyl]-4-methylpyridin-3-yl]-1-methyl-1,6-naphthyridin-2-one (Compound C-14) (530.0 mg, 85%) as a yellow solid. LCMS (ESI, m/z): [M+H]⁺=330.1.

### Example 19. Synthesis of Intermediate C′-15: 3-(6-(2-((tert-butyldimethylsilyl)oxy)propan-2-yl)-4-methylpyridin-3-yl)-7-chloro-1-methyl-1,6-naphthyridin-2(1H)-one

### Step 1: Synthesis of 2-(5-bromo-4-methylpyridin-2-yl)propan-2-ol

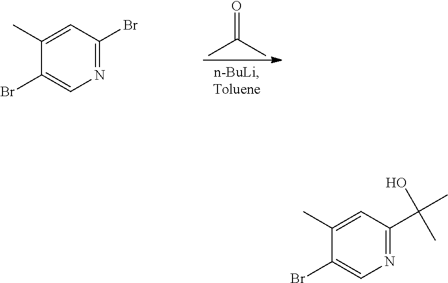

To a solution of 2,5-dibromo-4-methylpyridine (8.0 g, 31.88 mmol) in toluene (100.0 mL) was added n-BuLi (14.0 g, 35.7 mmol) dropwise at −78° C. under N₂. The resulting mixture was stirred at −78° C. for 0.5 h. Then acetone (7.4 g, 127.53 mmol) was added dropwise to the mixture at −78° C. under N₂. The resulting mixture was stirred at room temperature for 16 h. After the reaction was completed, the reaction was quenched with saturated NH₄Cl solution and then extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford 2-(5-bromo-4-methylpyridin-2-yl)propan-2-ol (5.0 g, 68%) as a yellow oil. LCMS (ESI, m/z): [M+H]⁺=230.0.

### Step 2: Synthesis of 5-bromo-2-(2-((tert-butyldimethylsilyl)oxy)propan-2-yl)-4-methylpyridine

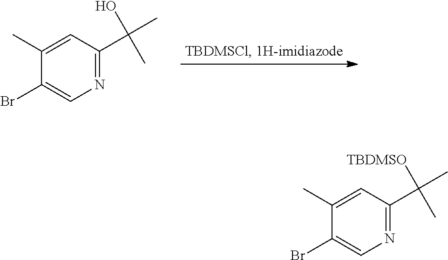 [SMILES: Cc1cc(C(C)(C)O)ncc1Br ; Cc1cc(C(C)(C)O[Si](C)(C)C(C)(C)C)ncc1Br]

To a solution of 2-(5-bromo-4-methylpyridin-2-yl)propan-2-ol (1.5 g, 6.52 mmol) in CH₂Cl₂ (20.0 mL) was added TBDMSCl (1.5 g, 9.78 mmol), 1H-imidazole (976.3 mg, 14.34 mmol) and 2,6-lutidine (1.4 g, 13.04 mmol) at room temperature under N₂. The resulting mixture was stirred at room temperature for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (12/1, v/v) to afford 5-bromo-2-(2-((tert-butyldimethylsilyl)oxy)propan-2-yl)-4-methylpyridine (1.8 g, 80%) as a colorless oil. LCMS (ESI, m/z): [M+H]⁺=344.1.

### Step 3: Synthesis of 2-(2-((tert-butyldimethylsilyl)oxy)propan-2-yl)-4-methyl-5-(4,4,5,5-tetramethyl-1,3,2-dioxaborolan-2-yl)pyridine (B-g′-14)

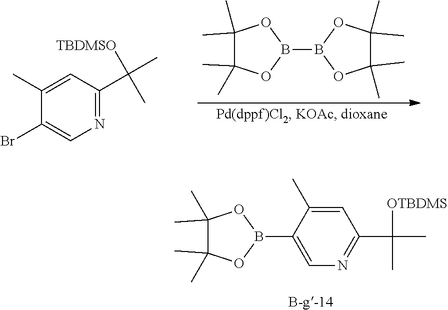

To a solution of 5-bromo-2-(2-((tert-butyldimethylsilyl)oxy)propan-2-yl)-4-methylpyridine (500.0 mg, 1.45 mmol) in dioxane (20.0 mL) was added 4,4,4′,4′,5,5,5′,5′-octamethyl-2,2′-bi(1,3,2-dioxaborolane) (1106.1 mg, 4.36 mmol), KOAc (427.5 mg, 4.36 mmol) and Pd(dppf)Cl₂ (106.2 mg, 0.15 mmol) at room temperature under N₂. The resulting mixture was stirred at 80° C. for 16 h. After the reaction was completed, the mixture was filtered. The filtrate was concentrated under reduced pressure to afford 2-(2-((tert-butyldimethylsilyl)oxy)propan-2-yl)-4-methyl-5-(4,4,5,5-tetramethyl-1,3,2-dioxaborolan-2-yl)pyridine (Compound B-g′-14) (600.0 mg, crude) as a brown solid. LCMS (ESI, m/z): [M+H]⁺=392.3.

### Step 4: Synthesis of 3-(6-(2-((tert-butyldimethylsilyl)oxy)propan-2-yl)-4-methylpyridin-3-yl)-7-chloro-1-methyl-1,6-naphthyridin-2(1H)-one (C′-15)

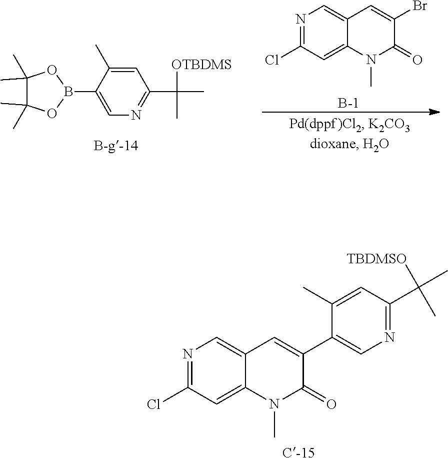

To a solution of 2-(2-((tert-butyldimethylsilyl)oxy)propan-2-yl)-4-methyl-5-(4,4,5,5-tetramethyl-1,3,2-dioxaborolan-2-yl)pyridine (Compound B-g′-14) (300.0 mg, crude) in dioxane/H₂O (10.0/2.0 mL) was added 3-bromo-7-chloro-1-methyl-1,6-naphthyridin-2(1H)-one (Compound B-1) (209.6 mg, 0.76 mmol), K₂CO₃ (317.8 mg, 2.30 mmol) and Pd(dppf)Cl₂ (56.1 mg, 0.08 mmol) at room temperature under N₂. The resulting mixture was stirred at 80° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (3/1, v/v) to afford 3-(6-(2-((tert-butyldimethylsilyl)oxy)propan-2-yl)-4-methylpyridin-3-yl)-7-chloro-1-methyl-1,6-naphthyridin-2(1H)-one (Compound C′-15) (83.0 mg, 23%) as a white solid. LCMS (ESI, m/z): [M+H]⁺=458.2.

### Example 20. Synthesis of Intermediate C-16: 7-chloro-3-[6-[(1S)-1-hydroxypropyl]-4-methylpyridin-3-yl]-1-methyl-1,6-naphthyridin-2-one

### Step 1: Synthesis of (1S)-1-[4-methyl-5-(4,4,5,5-tetramethyl-1,3,2-dioxaborolan-2-yl)pyridin-2-yl]propan-1-ol (B-g-15)

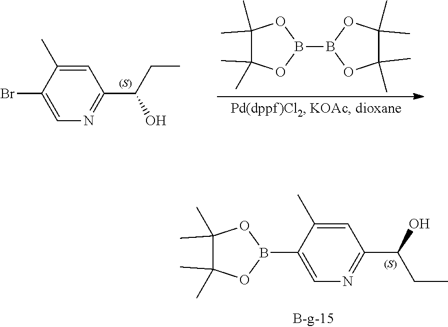

A mixture of (1S)-1-(5-bromo-4-methylpyridin-2-yl)propan-1-ol (1.1 g, 4.78 mmol), bis(pinacolato)diboron (1.5 g, 5.74 mmol), KOAc (1.4 g, 14.34 mmol) and Pd(dppf)Cl₂ (0.4 g, 0.48 mmol) in dioxane (35.0 mL) was stirred at 100° C. for 16 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (94/6, v/v) to afford (1S)-1-[4-methyl-5-(4,4,5,5-tetramethyl-1,3,2-dioxaborolan-2-yl)pyridin-2-yl]propan-1-ol (Compound B-g-15) (725.0 mg, 55%) as a yellow oil. LCMS (ESI, m/z): [M+H]⁺=278.2.

### Step 2: Synthesis of 7-chloro-3-[6-[(1S)-1-hydroxypropyl]-4-methylpyridin-3-yl]-1-methyl-1,6-naphthyridin-2-one (C-16)

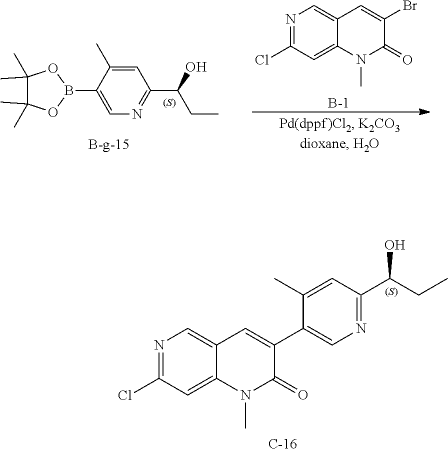

A mixture of (1S)-1-[4-methyl-5-(4,4,5,5-tetramethyl-1,3,2-dioxaborolan-2-yl)pyridin-2-yl]propan-1-ol (Compound B-g-15) (670.0 mg, 2.42 mmol), 3-bromo-7-chloro-1-methyl-1,6-naphthyridin-2-one (Compound B-1) (661.2 mg, 2.42 mmol), K₂CO₃ (1002.3 mg, 7.25 mmol) and Pd(dppf)Cl₂ (176.9 mg, 0.24 mmol) in dioxane (20.0 mL) and H₂O (4.0 mL) was stirred at 80° C. for 16 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (94/6, v/v) to afford 7-chloro-3-[6-[(1S)-1-hydroxypropyl]-4-methylpyridin-3-yl]-1-methyl-1,6-naphthyridin-2-one (Compound C-16) (810.0 mg, 97%) as a yellow solid. LCMS (ESI, m/z): [M+H]⁺=344.1

### Example 21. Synthesis of Intermediate C-17: 7-chloro-1-ethyl-3-(5-fluoro-4-methylpyridin-3-yl)-1,6-naphthyridin-2-one

### Step 1: Synthesis of 3-bromo-7-chloro-1-ethyl-1,6-naphthyridin-2-one (B-3)

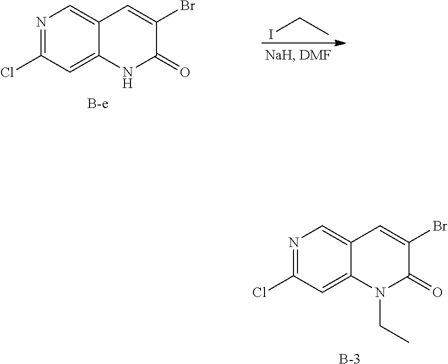

To a solution of 3-bromo-7-chloro-1H-1,6-naphthyridin-2-one (Compound B-e) (400.0 mg, 1.54 mmol) in DMF (10.0 mL) was added sodium hydride (123.1 mg, 60%) at 0° C. under N₂. The mixture was stirred at 0° C. for 30 min. Then ethyl iodide (480.8 mg, 3.08 mmol) was added to the mixture at 0° C. The mixture was stirred at room temperature for another 1 h. After the reaction was completed, the resulting mixture was quenched by H₂O and extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (60/40, v/v) to afford 3-bromo-7-chloro-1-ethyl-1,6-naphthyridin-2-one (Compound B-3) (190.0 mg, 43%) as a yellow solid. LCMS (ESI, m/z): [M+H]⁺=287.0.

### Step 2: 7-chloro-1-ethyl-3-(5-fluoro-4-methylpyridin-3-yl)-1,6-naphthyridin-2-one (C-17)

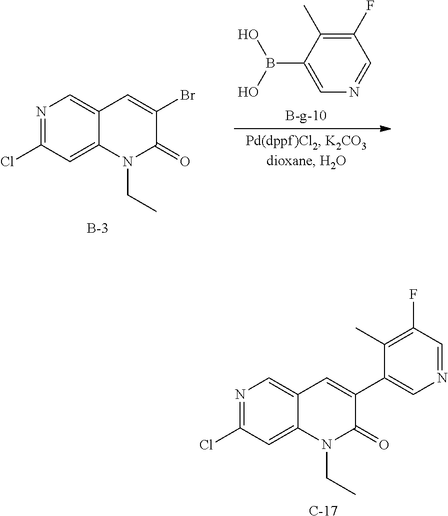

To a solution of 3-bromo-7-chloro-1-ethyl-1,6-naphthyridin-2-one (Compound B-3) (190.0 mg, 0.66 mmol) in 1,4-dioxane/H₂O (5.0/1.0 mL) was added 5-fluoro-4-methylpyridin-3-ylboronic acid (Compound B-g-10) (102.4 mg, 0.66 mmol), K₂CO₃ (274.0 mg, 1.98 mmol) and Pd(dppf)Cl₂ (48.4 mg, 0.07 mmol). The resulting mixture was stirred at 80° C. for 3 h under N₂. After the reaction was completed, the resulting mixture was concentrated under vacuum. The residue was purified by flash column chromatography with petroleum ether/ethyl acetate (65/35, v/v) to afford 7-chloro-1-ethyl-3-(5-fluoro-4-methylpyridin-3-yl)-1,6-naphthyridin-2-one (Compound C-17) (100.0 mg, 48%) as a yellow solid. LCMS (ESI, m/z): [M+H]⁺=318.1.

### Example 22. Synthesis of Intermediate B-g-16: (R)-(6-(1-hydroxypropyl)-4-methylpyridin-3-yl)boronic acid

### Step 1: Synthesis of 5-bromo-N-methoxy-N,4-dimethylpyridine-2-carboxamide

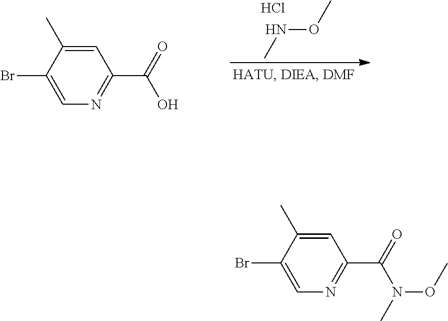

To a mixture of 5-bromo-4-methylpyridine-2-carboxylic acid (5.0 g, 23.14 mmol) and N,O-dimethylhydroxylamine hydrochloride (4.5 g, 46.28 mmol) in DMF (50.0 mL) was added HATU (17.6 g, 46.28 mmol) and DIEA (11.9 g, 92.57 mmol) at room temperature. The resulting mixture was stirred at room temperature for 2 h. After the reaction was completed, the resulting mixture was diluted with water and extracted with ethyl acetate. The combined organic layer was washed with brine, dried over anhydrous Na₂SO₄ and filtered. The filtrate was concentrated under vacuum. The residue was purified by flash column chromatography with petroleum ether/ethyl acetate (5/1, v/v) to afford 5-bromo-N-methoxy-N,4-dimethylpyridine-2-carboxamide (4.2 g, 87%) as an off-white solid. LCMS (ESI, m/z): [M+H]⁺=259.1.

### Step 2: Synthesis of 1-(5-bromo-4-methylpyridin-2-yl)propan-1-one

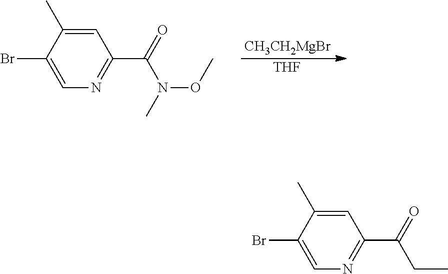

To a solution of 5-bromo-N-methoxy-N,4-dimethylpyridine-2-carboxamide (4.1 g, 15.82 mmol) in THE (40.0 mL) was added dropwise ethylmagnesium bromide (16.5 mL, 18.75 mmol) at 0° C. The above mixture was stirred at 0° C. for 2 h under N₂. After the reaction was completed, the resulting mixture was quenched by saturated NH₄Cl solution. The resulting mixture was diluted with water and extracted with ethyl acetate. The combined organic layer was washed with brine, dried over anhydrous Na₂SO₄ and filtered. The filtrate was concentrated under reduced pressure. The residue was purified by flash column chromatography with petroleum ether/ethyl acetate (8/1, v/v) to afford 1-(5-bromo-4-methylpyridin-2-yl)propan-1-one (2.1 g, 60%) as a white solid. LCMS (ESI, m/z): [M+H]⁺=228.0.

### Step 3: Synthesis of 1-(5-bromo-4-methylpyridin-2-yl)propan-1-ol

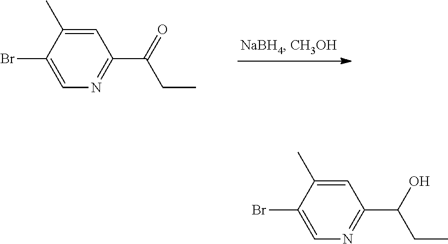

To a solution of 1-(5-bromo-4-methylpyridin-2-yl)propan-1-one (2.9 g, 12.7 mmol) in MeOH (20.0 mL) was added NaBH₄ (481.0 mg, 12.7 mmol) at 0° C. The resulting mixture was stirred at 0° C. for 2 h. After the reaction was completed, the resulting mixture was diluted with H₂O and extracted with ethyl acetate. The combined organic layer was washed with brine, dried over anhydrous Na₂SO₄ and filtered. The filtrate was concentrated under vacuum. The residue was purified by flash column chromatography with petroleum ether/ethyl acetate (7/1, v/v) to afford 1-(5-bromo-4-methylpyridin-2-yl)propan-1-ol (3.0 g, 97%) as a white solid. LCMS (ESI, m/z): [M+H]⁺=230.1

### Step 4: Synthesis of (1S)-1-(5-bromo-4-methylpyridin-2-yl)propan-1-ol and (1R)-1-(5-bromo-4-methylpyridin-2-yl)propyl acetate

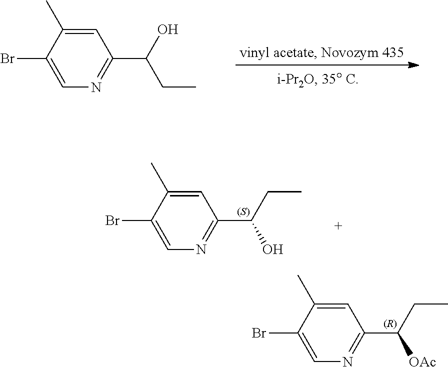

A mixture of 1-(5-bromo-4-methylpyridin-2-yl)propan-1-ol (2.9 g, 12.60 mmol) and ethenyl acetate (10.8 g, 126.03 mmol) and Novezym 435 (240.0 mg) in 2-(propan-2-yloxy)propane (25.0 mL) was stirred at 35° C. for 16 h. The resulting mixture was filtered. The filtrate was concentrated under vacuum. The residue was purified by flash column chromatography with petroleum ether/ethyl acetate (1/1, v/v) to afford (1R)-1-(5-bromo-4-methylpyridin-2-yl)propyl acetate (1.3 g, 40%) as a beige oil LCMS (ESI, m/z): [M+H]⁺=272.1 and (1S)-1-(5-bromo-4-methylpyridin-2-yl)propan-1-ol (1.4 g, 51%) as a beige oil, LCMS (ESI, m/z): [M+H]⁺=230.1.

### Step 5: Synthesis of (1R)-1-(5-bromo-4-methylpyridin-2-yl)propan-1-ol

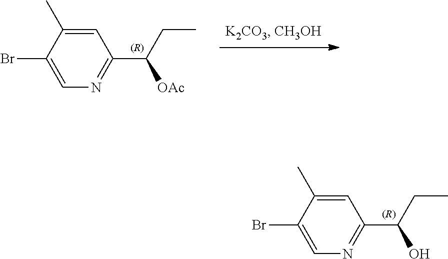

A mixture of (1R)-1-(5-bromo-4-methylpyridin-2-yl)propyl acetate (1.3 g, 5.07 mmol) and K₂CO₃ (1.4 g, 10.14 mmol) in CH₃OH (15.0 mL) was stirred at room temperature for 2 h. The resulting mixture was filtered. The filtrate was concentrated under vacuum. The residue was purified by flash column chromatography with petroleum ether/ethyl acetate (1/1, v/v) to afford (1R)-1-(5-bromo-4-methylpyridin-2-yl)propan-1-ol (790.0 mg, 67%) as a beige oil. LCMS (ESI, m/z): [M+H]⁺=230.1

### Step 6: Synthesis of (R)-(6-(1-hydroxypropyl)-4-methylpyridin-3-yl)boronic acid (B-g-16)

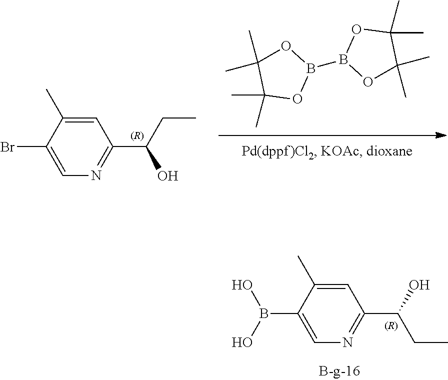

To a mixture of (1R)-1-(5-bromo-4-methylpyridin-2-yl)propan-1-ol (740.0 mg, 3.21 mmol), bis(pinacolato)diboron (979.9 mg, 3.85 mmol) and KOAc (946.8 mg, 9.64 mmol) in dioxane (15.0 mL) was added Pd(dppf)Cl₂ (470.6 mg, 0.64 mmol). The mixture was stirred at 100° C. for 16 h under N₂. After the reaction was completed, the resulting mixture was filtered. The filtrate was concentrated under reduced pressure. The residue was purified by reverse phase flash chromatography with ACN in water (5% to 100% gradient in 50 min) to afford (R)-(6-(1-hydroxypropyl)-4-methylpyridin-3-yl)boronic acid (Compound B-g-16) (520.0 mg, 58%) as a brown yellow solid. LCMS (ESI, m/z): [M+H]⁺=196.1.

### Example 23. Synthesis of Intermediate B-g-17: 7-chloro-1-methyl-3-(4-methylpyridin-3-yl)-1,6-naphthyridin-2-one

### Step 1: Synthesis of 5-bromo-N-methoxy-N,4-dimethylpyridine-2-carboxamide

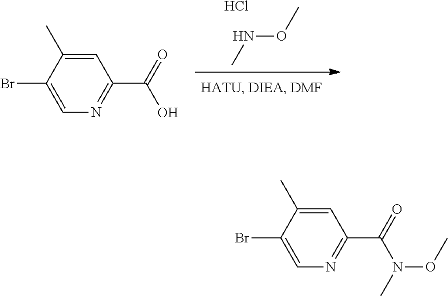

To a mixture of 5-bromo-4-methylpyridine-2-carboxylic acid (3.9 g, 18.05 mmol) and N,O-dimethylhydroxylamine hydrochloride (3.5 g, 36.10 mmol) in DMF (30.0 mL) were added HATU (13.7 g, 36.10 mmol) and DIEA (9.3 g, 72.21 mmol) at room temperature. The mixture was stirred at room temperature for 3 h. After the reaction was completed, the resulting mixture was diluted with water and extracted with ethyl acetate. The combined organic layer was washed with brine, dried over anhydrous Na₂SO₄ and filtered. The filtrate was concentrated under reduced pressure. The residue was purified by flash column chromatography with petroleum ether/ethyl acetate (8/1, v/v) to afford 5-bromo-N-methoxy-N,4-dimethylpyridine-2-carboxamide (4.2 g, 90%) as a white solid. LCMS (ESI, m/z): [M+H]⁺=259.0.

### Step 2: Synthesis of 1-(5-bromo-4-methylpyridin-2-yl)ethanone

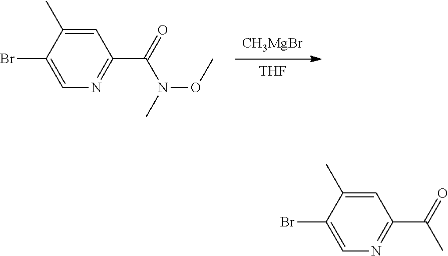

To a solution of 5-bromo-N-methoxy-N,4-dimethylpyridine-2-carboxamide (3.3 g, 12.92 mmol) in tetrahydrofuran (30.0 mL) was added dropwise bromo(methyl)magnesium (6.5 mL, 19.37 mmol) at 0° C. under N₂. The resulting mixture was stirred at 0° C. for 2 h. The reaction was quenched with sat. NH₄Cl (aq.). The resulting mixture was extracted with ethyl acetate. The combined organic layers were washed with water, dried over anhydrous Na₂SO₄ and filtered. The filtrate was concentrated under reduced pressure. The residue was purified by flash column chromatography with petroleum ether/ethyl acetate (1/1, v/v) to afford 1-(5-bromo-4-methylpyridin-2-yl)ethanone (2.4 g, 87%) as a white solid. LCMS (ESI, m/z): [M+H]⁺=214.1.

### Step 3: Synthesis of 1-(5-bromo-4-methylpyridin-2-yl)ethanol

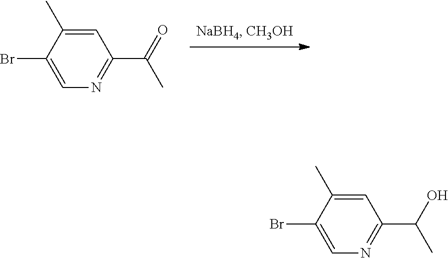

To a solution of 1-(5-bromo-4-methylpyridin-2-yl)ethanone (2.4 g, 11.352 mmol) in MeOH (30.0 mL) was added NaBH₄ (429.4 mg, 11.35 mmol) at 0° C. The mixture was stirred at 0° C. for 30 min. After the reaction was completed, the mixture was diluted with water and extracted with ethyl acetate. The combined organic layer was washed with brine, dried over anhydrous sodium sulfate and filtered. The filtrate was concentrated under vacuum to afford 1-(5-bromo-4-methylpyridin-2-yl)ethanol (2.5 g, crude). LCMS (ESI, m/z): [M+H]⁺=216.1.

### Step 4: Synthesis of (S)-1-(5-bromo-4-methylpyridin-2-yl)ethanol

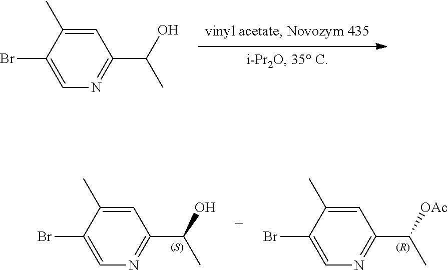

To a mixture of 1-(5-bromo-4-methylpyridin-2-yl)ethanol (2.5 g, 11.57 mmol) and ethenyl acetate (9.9 g, 115.69 mmol) in 2-(propan-2-yloxy)propane (30.0 mL) was added Novezy m435 (200.0 mg). The resulting mixture was stirred at 35° C. for 16 h. After the reaction was completed, the resulting mixture was filtered. The filtrate was concentrated under reduced pressure. The residue was by flash column chromatography with petroleum ether/ethyl acetate (1/1, v/v) to afford (1S)-1-(5-bromo-4-methylpyridin-2-yl)ethanol (920.0 mg, 37%) as a beige oil and (1R)-1-(5-bromo-4-methylpyridin-2-yl)ethyl acetate (900.0 mg, 30%) as a beige oil. LCMS (ESI, m/z): [M+H]⁺=216.1.

### Step 5: Synthesis of 7-chloro-1-methyl-3-(4-methylpyridin-3-yl)-1,6-naphthyridin-2-one

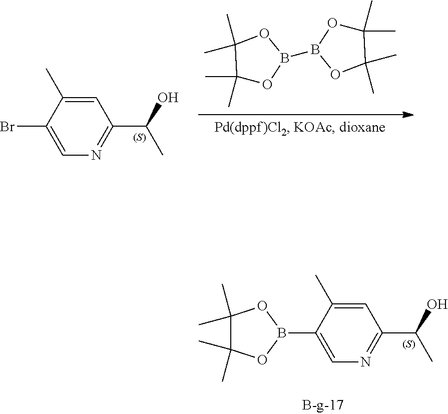

A mixture of (1S)-1-(5-bromo-4-methylpyridin-2-yl)ethanol (800.0 mg, 3.70 mmol), bis(pinacolato)diboron (1.1 g, 4.44 mmol), KOAc (1.0 g, 11.10 mmol) and Pd(dppf)Cl₂ (541.8 mg, 0.74 mmol) in dioxane (15.0 mL) was stirred at 80° C. for 16 h under N₂. After the reaction was completed, the resulting mixture was diluted with H₂O and extracted with ethyl acetate. The combined organic layer was washed with brine, dried over anhydrous Na₂SO₄ and filtered. The filtrate was concentrated under reduced pressure. The residue was purified by reverse phase flash column chromatography with ACN in water (10% to 50% gradient in 30 min) to afford (1S)-1-[4-methyl-5-(4,4,5,5-tetramethyl-1,3,2-dioxaborolan-2-yl)pyridin-2-yl]ethanol (Compound B-g-17) (500.0 mg, 51%) as a light yellow oil. LCMS (ESI, m/z): [M+H]⁺=264.1.

### Example 24. Synthesis of Intermediate B-g-18: 5-cyano-4-methylpyridin-3-ylboronic acid

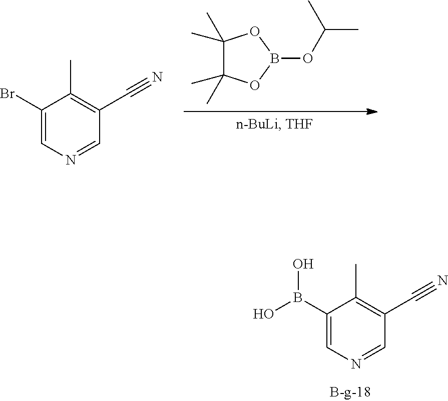

To a mixture of 5-bromo-4-methylpyridine-3-carbonitrile (601.0 mg, 3.05 mmol) and 2-isopropoxy-4,4,5,5-tetramethyl-1,3,2-dioxaborolane (827.0 mg, 4.44 mmol) in THF (12.0 mL) was dropwise added n-BuLi in hexanes (1.5 mL, 2.5 mol/L) at −70° C. under N₂. The mixture was stirred at −70° C. for 2 h. After the reaction was completed, the pH value of the mixture was adjusted to 5 with HCl (1 mol/L). The mixture was dried over anhydrous sodium sulfate and filtered. The filtrate was concentrated under vacuum to afford 5-cyano-4-methylpyridin-3-ylboronic acid (Compound B-g-18) (710.0 mg, crude) as a brown oil. LCMS (ESI, m/z): [M+H]⁺=163.1.

### Example 25. Synthesis of Intermediate B-g-19: 6-(1-hydroxypropyl)-4-methylpyridin-3-ylboronic acid

### Step 1: Synthesis of 1-(5-bromo-4-methylpyridin-2-yl)propan-1-ol

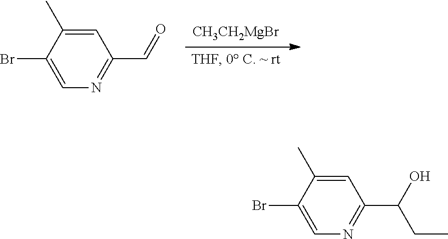

To a mixture of 5-bromo-4-methylpyridine-2-carbaldehyde (2.5 g, 12.49 mmol) in THE (35.0 mL) was added dropwise ethylmagnesium bromide (18.7 mL, 1 mol/L) at 0° C. under N₂ for 30 min. The resulting mixture was stirred at room temperature for 2 h. After the reaction was completed, the reaction mixture was quenched with sat. NH₄Cl (aq.) at 0° C. The reaction mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (2/1, v/v) to afford 1-(5-bromo-4-methylpyridin-2-yl)propan-1-ol (1.1 g, 38%) as a yellow oil. LCMS (ESI, m/z): [M+H]⁺=230.1.

### Step 2: Synthesis of 6-(1-hydroxypropyl)-4-methylpyridin-3-ylboronic acid

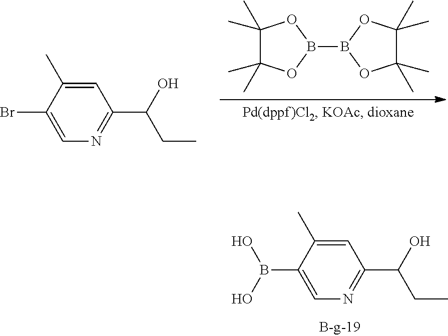

To a solution of 1-(5-bromo-4-methylpyridin-2-yl)propan-1-ol (900.0 mg, 3.91 mmol) in dioxane (15.0 mL) was added bis(pinacolato)diboron (1489.8 mg, 5.88 mmol), KOAc (767.7 mg, 7.82 mmol) and Pd(dppf)Cl₂ (286.2 mg, 0.39 mmol) at room temperature under N₂. The resulting mixture was stirred at 120° C. for 1 h. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure. The residue was purified by reverse phase flash chromatography with ACN/H₂O (30/70, v/v) to afford 6-(1-hydroxypropyl)-4-methylpyridin-3-ylboronic acid (Compound B-g-19) (500.0 mg, 65%) as a light yellow solid. LCMS (ESI, m/z): [M+H]⁺=196.1.

### Example 26. Synthesis of Intermediate D-5: (1R,2S)-2-fluorocyclopropane-1-carboxamide

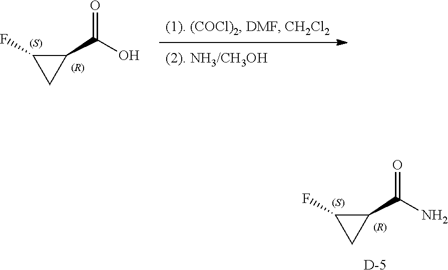

To a solution of (1R,2S)-2-fluorocyclopropane-1-carboxylic acid (1.0 g, 9.61 mmol) in CH₂Cl₂ (10.0 mL) was added DMF (0.1 mL) and oxalyl dichloride (1.3 g, 10.57 mmol) at room temperature. The resulting mixture was stirred at room temperature for 1 h. Then NH₃/CH₃OH (10.0 mL) was added to the mixture at room temperature. The resulting mixture was stirred at room temperature for another 10 min. After the reaction was completed, the resulting mixture was concentrated under reduced pressure to afford (1R,2S)-2-fluorocyclopropane-1-carboxamide (Compound D-5) (900.0 mg, crude) as a white solid. LCMS (ESI, m/z): [M+H]⁺=104.0.

### Example 27. Synthesis of Intermediate D-6: (1S,2R)-2-fluorocyclopropane-1-carboxamide

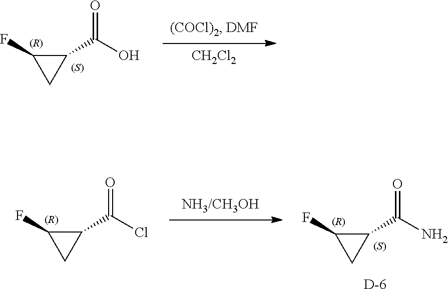

To a solution of (1S,2R)-2-fluorocyclopropane-1-carboxylic acid (1.0 g, 9.61 mmol) in CH₂Cl₂ (20.0 mL) was added DMF (0.1 mL) and oxalyl dichloride (1.3 g, 10.56 mmol) at room temperature. The resulting mixture was stirred at room temperature for 1 h. Then NH₃/MeOH (20.0 mL, 7 mol/L) was added to the mixture at room temperature. The resulting mixture was stirred at room temperature for another 10 min. After the reaction was completed, the resulting mixture was concentrated under vacuum to afford (1S,2R)-2-fluorocyclopropane-1-carboxamide (Compound D-6) (1.03 g, crude) as a white solid. LCMS (ESI, m/z): [M+H]⁺=104.1.

### Example 28. Synthesis of Intermediate C-18: 7-chloro-3-[6-[(1R)-1-hydroxypropyl]-4-methylpyridin-3-yl]-1-methyl-1,6-naphthyridin-2-one

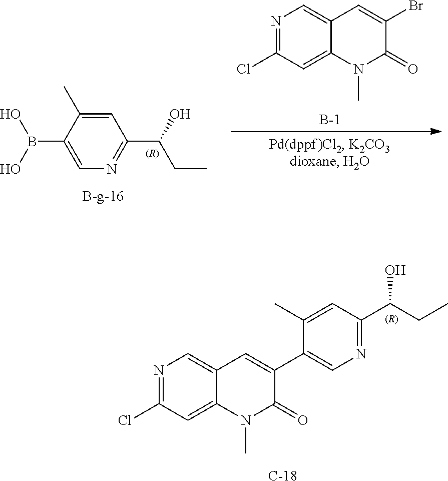

A mixture of (R)-(6-(1-hydroxypropyl)-4-methylpyridin-3-yl)boronic acid (Compound B-16-g) (470.0 mg, 2.41 mmol), 3-bromo-7-chloro-1-methyl-1,6-naphthyridin-2-one (Compound B-1) (659.1 mg, 2.41 mmol), K₂CO₃ (999.1 mg, 7.23 mmol) and Pd(dppf)Cl₂ (352.6 mg, 0.48 mmol) in dioxane (10.0 mL)/H₂O (1.0 mL) was stirred at 80° C. for 16 h under N₂. After the reaction was completed, the resulting mixture was concentrated under reduced pressure. The residue was purified by flash column chromatography with petroleum ether/ethyl acetate (7/3, v/v) to afford 7-chloro-3-[6-[(1R)-1-hydroxypropyl]-4-methylpyridin-3-yl]-1-methyl-1,6-naphthyridin-2-one (Compound C-18) (400.0 mg, 48%) as a brown solid. LCMS (ESI, m/z): [M+H]⁺=344.1.

### Example 29. Synthesis of Intermediate C-19: 7-chloro-1-(cyclopropylmethyl)-3-(5-fluoro-4-methylpyridin-3-yl)-1,6-naphthyridin-2-one

### Step 1: Synthesis of 3-bromo-7-chloro-1-(cyclopropylmethyl)-1,6-naphthyridin-2-one

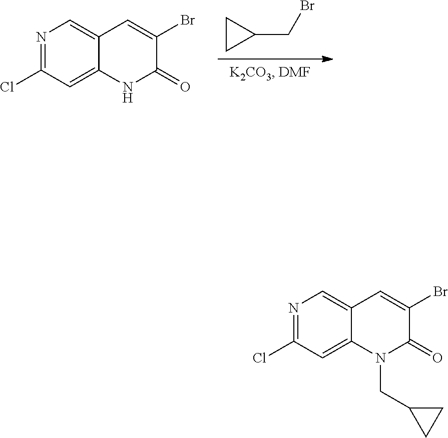

To a solution of 3-bromo-7-chloro-1H-1,6-naphthyridin-2-one (1.5 g, 5.78 mmol) in DMF (20.0 mL) was added K₂CO₃ (4.0 g, 28.90 mmol) and (bromomethyl)cyclopropane (1.6 g, 11.56 mmol). The mixture was stirred at room temperature for 16 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (80/20, v/v) to afford 3-bromo-7-chloro-1-(cyclopropylmethyl)-1,6-naphthyridin-2-one (630.0 mg, 35%) as a yellow solid. LCMS (ESI, m/z): [M+H]⁺=313.0.

### Step 2: Synthesis of 7-chloro-1-(cyclopropylmethyl)-3-(5-fluoro-4-methylpyridin-3-yl)-1,6-naphthyridin-2-one

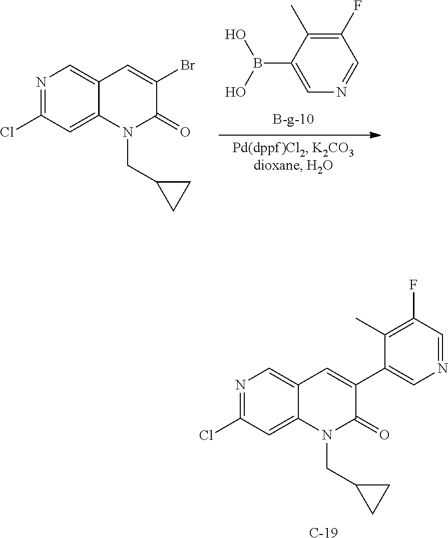

To a solution of 3-bromo-7-chloro-1-(cyclopropylmethyl)-1,6-naphthyridin-2-one (630.0 mg, 2.01 mmol) in 1,4-dioxane/H₂O (15.0/3.0 mL) was added 5-fluoro-4-methylpyridin-3-ylboronic acid (Compound B-g-10) (311.3 mg, 2.01 mmol), K₂CO₃ (833.0 mg, 6.03 mmol) and Pd(dppf)Cl₂ (147.0 mg, 0.20 mmol). The resulting mixture was stirred at 80° C. for 16 h under N₂. After the reaction was completed, the resulting mixture was concentrated under vacuum. The residue was purified by flash column chromatography with CH₂Cl₂/MeOH (95/5, v/v) to afford 7-chloro-1-(cyclopropylmethyl)-3-(5-fluoro-4-methylpyridin-3-yl)-1,6-naphthyridin-2-one (Compound C-19) (200.0 mg, 29%) as a yellow solid. LCMS (ESI, m/z): [M+H]⁺=344.1.

### Example 30. Synthesis of Intermediate C-20: 7-chloro-3-[6-[(1S)-1-hydroxyethyl]-4-methylpyridin-3-yl]-1-methyl-1,6-naphthyridin-2-one

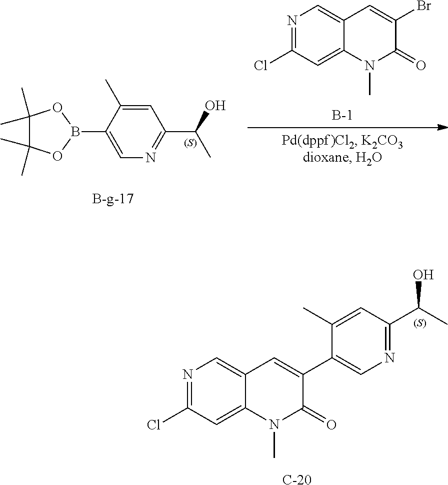

A mixture of (1S)-1-[4-methyl-5-(4,4,5,5-tetramethyl-1,3,2-dioxaborolan-2-yl)pyridin-2-yl]ethanol (Compound B-g-17) (500.0 mg, 2.76 mmol), 3-bromo-7-chloro-1-methyl-1,6-naphthyridin-2-one (Compound B-1) (604.4 mg, 2.21 mmol), K₂CO₃ (1145.3 mg, 8.28 mmol) and Pd(dppf)Cl₂ (404.2 mg, 0.55 mmol) in dioxane (8.0 mL)/H₂O (0.8 mL) was stirred at 80° C. for 16 h under N₂. After the reaction was completed, the resulting mixture was concentrated under vacuum. The residue was purified by flash column chromatography with CH₂Cl₂/MeOH (9/1, v/v) to afford 7-chloro-3-[6-[(1S)-1-hydroxyethyl]-4-methylpyridin-3-yl]-1-methyl-1,6-naphthyridin-2-one (Compound C-20) (560.0 mg, 61%) as a brown yellow solid. LCMS (ESI, m/z): [M+H]⁺=330.1.

### Example 31. Synthesis of Intermediate C-21: 5-(7-chloro-1-methyl-2-oxo-1,6-naphthyridin-3-yl)-4-methylpyridine-3-carbonitrile

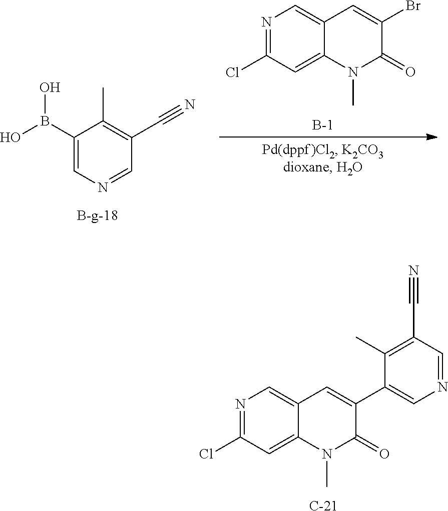

A mixture of 3-bromo-7-chloro-1-methyl-1,6-naphthyridin-2-one (Compound B-1) (328.7 mg, 1.20 mmol), 5-cyano-4-methylpyridin-3-ylboronic acid (Compound B-g-18) (710.0 mg, crude), Pd(dppf)Cl₂ (113.1 mg, 0.15 mmol) and K₂CO₃ (473.8 mg, 3.42 mmol) in dioxane (8.0 mL) and H₂O (2.0 mL) was heated at 100° C. for 16 h. The mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (90/10, v/v) to afford 5-(7-chloro-1-methyl-2-oxo-1,6-naphthyridin-3-yl)-4-methylpyridine-3-carbonitrile (Compound C-21) (140.0 mg, 37%) as a light yellow solid. LCMS (ESI, m/z): [M+H]⁺=311.1.

### Example 32. Synthesis of Intermediate C-22: 7-chloro-3-[6-(1-hydroxypropyl)-4-methylpyridin-3-yl]-1H-1,6-naphthyridin-2-one

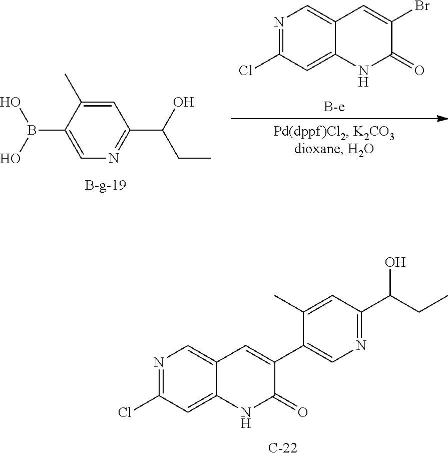

To a solution of 6-(1-hydroxypropyl)-4-methylpyridin-3-ylboronic acid (Compound B-g-19) (400.0 mg, 2.05 mmol) in dioxane (20.0 mL)/H₂O (0.4 mL) was added K₂CO₃ (42.5 mg, 0.31 mmol), 3-bromo-7-chloro-1H-1,6-naphthyridin-2-one (Compound B-e) (532.2 mg, 2.05 mmol) and Pd(dppf)Cl₂ (150.1 mg, 0.21 mmol) at room temperature under N₂. The final reaction mixture was irradiated with microwave radiation at 120° C. for 1.5 h under N₂. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure. The residue was purified by flash chromatography with CH₂Cl₂/MeOH (5/1, v/v) to afford 7-chloro-3-[6-(1-hydroxypropyl)-4-methylpyridin-3-yl]-1H-1,6-naphthyridin-2-one (Compound C-22) (450.0 mg, 66%) as a light yellow solid. LCMS (ESI, m/z): [M+H]⁺=330.1.

### Example S1. Synthesis of N-[1-methyl-3-(4-methylpyridin-3-yl)-2-oxo-1,6-naphthyridin-7-yl]cyclopropanecarboxamide (Compound 1)

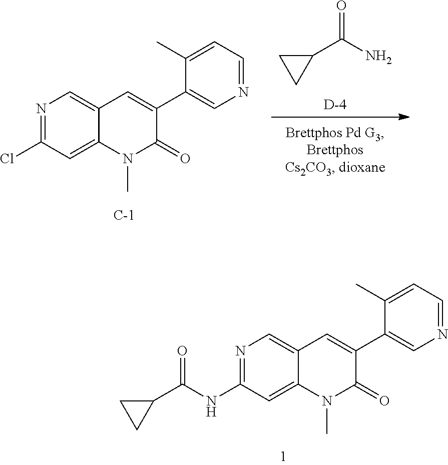

To a stirred solution of 7-chloro-1-methyl-3-(4-methylpyridin-3-yl)-1,6-naphthyridin-2-one (Compound C-1) (200.0 mg, 0.70 mmol) in 1,4-dioxane (10.0 mL) was added cyclopropanecarboxamide (Compound D-4) (178.7 mg, 2.10 mmol), BrettPhos (75.1 mg, 0.14 mmol), Cs₂CO₃ (684.2 mg, 2.10 mmol) and BrettPhos Pd G3 (63.5 mg, 0.07 mmol) at room temperature under N₂. The resulting mixture was stirred at 100° C. for 2 h under N₂. After the reaction was completed, the resulting mixture was diluted with H₂O and extracted with ethyl acetate. The combined organic layer was washed with brine, dried over anhydrous sodium sulfate, and filtered. The filtrate was concentrated under vacuum. The residue was purified by flash column chromatography with CH₂Cl₂/CH₃OH (94/6, v/v) and then purified by Prep-HPLC with the following conditions (Column: YMC-Actus Triart C18 Column, 30×250 mm, 5 um; Mobile Phase A: Water (10 mmol/L NH₄HCO₃), Mobile Phase B: ACN; Flow rate: 60 mL/min; Gradient: 18% B to 42% B in 8 min; 254 nm) to afford N-[1-methyl-3-(4-methylpyridin-3-yl)-2-oxo-1,6-naphthyridin-7-yl]cyclopropanecarboxamide (Compound 1) (120.5 mg, 51%) as a white solid. LCMS (ESI, m/z): [M+H]⁺=335.2. ¹H NMR (300 MHz, DMSO-d₆) δ 11.17 (s, 1H), 8.72 (s, 1H), 8.46 (d, J=4.8 Hz, 1H), 8.37 (s, 1H), 8.26 (s, 1H), 8.02 (s, 1H), 7.33 (d, J=5.1 Hz, 1H), 3.60 (s, 3H), 2.20 (s, 3H), 2.12-2.07 (m, 1H), 0.89-0.86 (m, 4H).

### Example S2. Synthesis of N-[3-(5-fluoro-4-methylpyridin-3-yl)-1-methyl-2-oxo-1,6-naphthyridin-7-yl]cyclopropanecarboxamide (Compound 2)

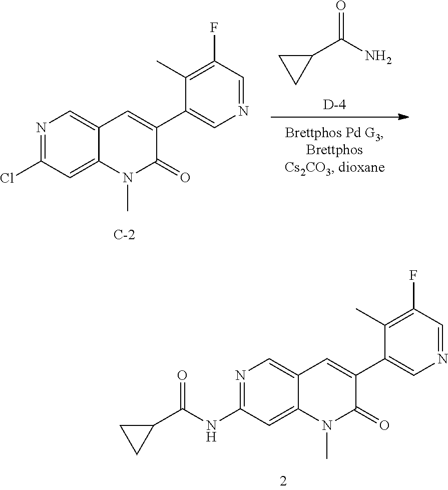

To a stirred solution of 7-chloro-3-(5-fluoro-4-methylpyridin-3-yl)-1-methyl-1,6-naphthyridin-2-one (Compound C-2) (80.0 mg, 0.26 mmol) in 1,4-dioxane (3.0 mL) was added cyclopropanecarboxamide (Compound D-4) (67.3 mg, 0.79 mmol), BrettPhos (28.3 mg, 0.05 mmol), Cs₂CO₃ (257.5 mg, 0.79 mmol) and BrettPhos Pd G3 (23.9 mg, 0.03 mmol) at room temperature under N₂. The resulting mixture was stirred at 100° C. for 2 h under N₂. After the reaction was completed, the resulting mixture was diluted with H₂O and extracted with ethyl acetate. The combined organic layer was washed with brine, dried over anhydrous sodium sulfate, and filtered. The filtrate was concentrated under vacuum. The residue was purified by flash column chromatography with CH₂Cl₂/CH₃OH (94/6, v/v) and then purified by Prep-HPLC with the following conditions (Column: Xselect CSH OBD Column, 30×150 mm, 5 um; Mobile Phase A: Water (10 mmol/L NH₄HCO₃), Mobile Phase B: ACN; Flow rate: 60 mL/min; Gradient: 25% B to 41% B in 8 min; 254 nm) to afford N-[3-(5-fluoro-4-methylpyridin-3-yl)-1-methyl-2-oxo-1,6-naphthyridin-7-yl]cyclopropanecarboxamide (Compound 2) (17.2 mg, 19%) as a white solid. LCMS (ESI, m/z): [M+H]⁺=353.1. ¹H NMR (300 MHz, DMSO-d₆): δ 11.19 (s, 1H), 8.73 (s, 1H), 8.53 (s, 1H), 8.30-8.27 (m, 2H), 8.08 (s, 1H), 3.60 (s, 3H), 2.14-2.10 (m, 4H), 0.89-0.87 (m, 4H).

### Example S3. Synthesis of N-[1-methyl-3-(2-methylpyridin-3-yl)-2-oxo-1,6-naphthyridin-7-yl]cyclopropanecarboxamide (Compound 3)

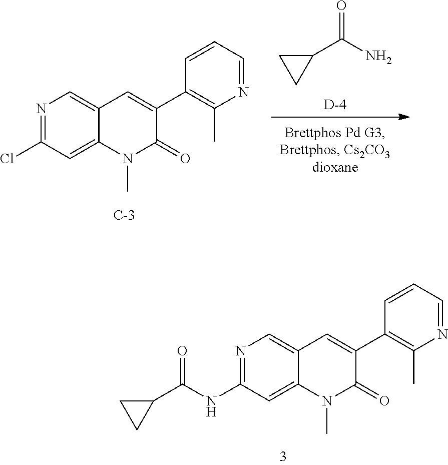

To a solution of 7-chloro-1-methyl-3-(2-methylpyridin-3-yl)-1,6-naphthyridin-2-one (Compound C-3) (300.0 mg, 1.05 mmol) in dioxane (10.0 mL) was added cyclopropanecarboxamide (Compound D-4) (268.1 mg, 3.15 mmol), BrettPhos (112.7 mg, 0.21 mmol), Cs₂CO₃ (1026.3 mg, 3.15 mmol) and BrettPhos Pd G3 (95.2 mg, 0.11 mmol) at room temperature under N₂. The resulting mixture was stirred at 100° C. for 16 h. After the reaction was completed, the resulting mixture was diluted with H₂O and extracted with ethyl acetate. The combined organic layer was washed with brine, dried over anhydrous sodium sulfate, and filtered. The filtrate was concentrated under reduced pressure. The residue was purified by flash column chromatography with petroleum ether/ethyl acetate (50/50, v/v) to afford N-[1-methyl-3-(2-methylpyridin-3-yl)-2-oxo-1,6-naphthyridin-7-yl]cyclopropanecarboxamide (Compound 3) (95.1 mg, 27%) as a white solid. LCMS (ESI, m/z): [M+H]⁺=335.2. ¹H NMR (300 MHz, DMSO-d₆): δ 11.17 (s, 1H), 8.70 (s, 1H), 8.48 (d, J=3.6 Hz, 1H), 8.25 (s, 1H), 7.99 (s, 1H), 7.64-7.63 (m, 1H), 7.31-7.27 (m, 1H), 3.59 (s, 3H), 2.35 (s, 3H), 2.13-2.07 (m, 1H), 0.90-0.83 (m, 4H).

### Example S4. Synthesis of N-[3-(5-methoxy-4-methylpyridin-3-yl)-1-methyl-2-oxo-1,6-naphthyridin-7-yl]cyclopropanecarboxamide (Compound 4)

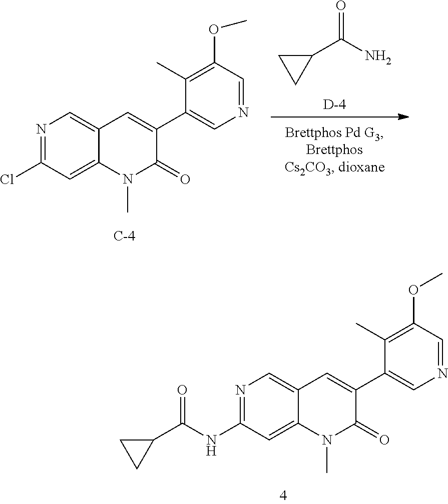

To a stirred solution of 7-chloro-3-(5-methoxy-4-methylpyridin-3-yl)-1-methyl-1,6-naphthyridin-2-one (Compound C-4) (50.00 mg, 0.16 mmol) in 1,4-dioxane (3.0 mL) was added cyclopropanecarboxamide (Compound D-4) (40.4 mg, 0.48 mmol), BrettPhos (17.0 mg, 0.03 mmol), Cs₂CO₃ (154.8 mg, 0.48 mmol) and BrettPhos Pd G3 (14.4 mg, 0.02 mmol) at room temperature under N₂. The resulting mixture was stirred at 100° C. for 2 h under N₂. After the reaction was completed, the resulting mixture was concentrated under vacuum. The residue was purified by flash column chromatography with CH₂Cl₂/MeOH (94/6, v/v) to afford N-[3-(5-methoxy-4-methylpyridin-3-yl)-1-methyl-2-oxo-1,6-naphthyridin-7-yl]cyclopropanecarboxamide (Compound 4) (17.2 mg, 18%) as a white solid. LCMS (ESI, m/z): [M+H]⁺=365.2. ¹H NMR (300 MHz, DMSO-d₆): δ 11.17 (s, 1H), 8.71 (s, 1H), 8.31 (s, 1H), 8.25 (s, 1H), 8.05 (s, 1H), 7.99 (s, 1H), 3.95 (s, 3H), 3.58 (s, 3H), 2.13-2.05 (m, 1H), 2.01 (s, 3H), 0.88-0.86 (m, 4H).

### Example S5. Synthesis of N-[3-(5-hydroxy-4-methylpyridin-3-yl)-1-methyl-2-oxo-1,6-naphthyridin-7-yl]cyclopropanecarboxamide (Compound 5)

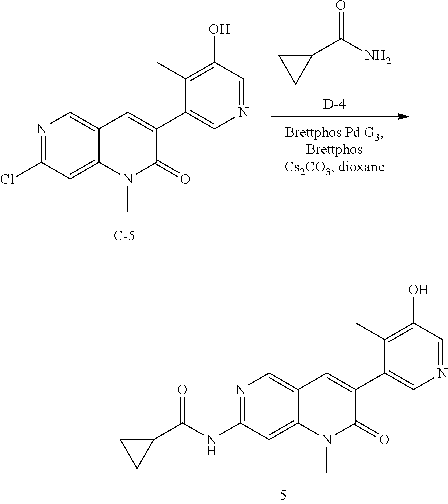

To a stirred solution of 7-chloro-3-(5-hydroxy-4-methylpyridin-3-yl)-1-methyl-1,6-naphthyridin-2-one (Compound C-5) (80.0 mg, 0.30 mmol) in 1,4-dioxane (3.0 mL) was added cyclopropanecarboxamide (Compound D-4) (76.4 mg, 0.90 mmol), BrettPhos (32.1 mg, 0.06 mmol), Cs₂CO₃ (292.6 mg, 0.90 mmol) and BrettPhos Pd G3 (27.1 mg, 0.03 mmol) at room temperature under N₂. The resulting mixture was stirred at 100° C. for 2 h under N₂. After the reaction was completed, the resulting mixture was concentrated under vacuum. The residue was purified by flash column chromatography with CH₂Cl₂/MeOH (94/6, v/v) to afford N-[3-(5-hydroxy-4-methylpyridin-3-yl)-1-methyl-2-oxo-1,6-naphthyridin-7-yl]cyclopropanecarboxamide (Compound 5) (15.4 mg, 15%) as a white solid. LCMS (ESI, m/z): [M+H]⁺=351.2. ¹H NMR (300 MHz, DMSO-d₆): δ 11.17 (s, 1H), 9.87 (s, 1H), 8.71 (s, 1H), 8.25 (s, 1H), 8.12 (s, 1H), 7.97 (s, 1H), 7.87 (s, 1H), 3.59 (s, 3H), 2.11-1.98 (m, 4H), 0.89-0.86 (m, 4H).

### Example S6. Synthesis of N-[3-(2,4-dimethylpyridin-3-yl)-1-methyl-2-oxo-1,6-naphthyridin-7-yl]cyclopropanecarboxamide (Compound 6)

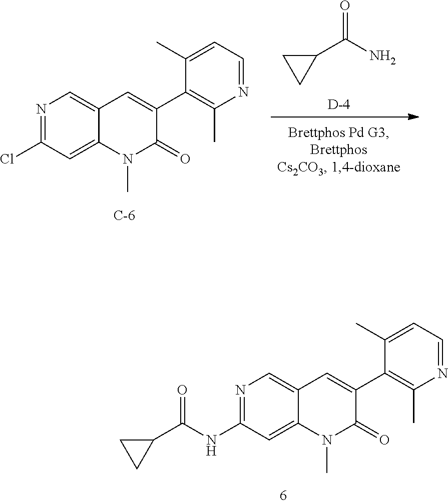

To a stirred solution of 7-chloro-3-(2,4-dimethylpyridin-3-yl)-1-methyl-1,6-naphthyridin-2-one (Compound C-6) (120.0 mg, 0.40 mmol) in 1,4-dioxane (5.0 mL) was added cyclopropanecarboxamide (Compound D-4) (102.2 mg, 1.20 mmol), BrettPhos (43.0 mg, 0.08 mmol), Cs₂CO₃ (391.30 mg, 1.201 mmol) and BrettPhos Pd G3 (36.3 mg, 0.04 mmol). The resulting mixture was stirred at 100° C. for 2 h under N₂. After the reaction was completed, the resulting mixture was concentrated under vacuum. The residue was purified by flash column chromatography with CH₂Cl₂/MeOH (93/7, v/v) and then purified by Prep-HPLC with the following conditions (Column: YMC-Actus Triart C18 Column, 30×250 mm, 5 um; Mobile Phase A: Water (10 mmol/L NH₄HCO₃), Mobile Phase B: ACN; Flow rate:60 mL/min; Gradient:22% B to 40% B in 7 min; 254 nm) to afford N-[3-(2,4-dimethylpyridin-3-yl)-1-methyl-2-oxo-1,6-naphthyridin-7-yl]cyclopropanecarboxamide (Compound 6) (16.3 mg, 70%) as a white solid. LCMS (ESI, m/z): [M+H]⁺=349.2. ¹H NMR (300 MHz, DMSO-d₆): δ 11.17 (s, 1H), 8.68 (s, 1H), 8.33-8.26 (m, 2H), 7.93 (s, 1H), 7.17 (d, J=5.1 Hz, 1H), 3.60 (s, 3H), 2.25 (s, 3H), 2.09-2.07 (m, 4H), 0.89-0.86 (m, 4H).

### Example S7. Synthesis of N-[3-(2,6-dimethylpyridin-3-yl)-1-methyl-2-oxo-1,6-naphthyridin-7-yl]cyclopropanecarboxamide (Compound 7)

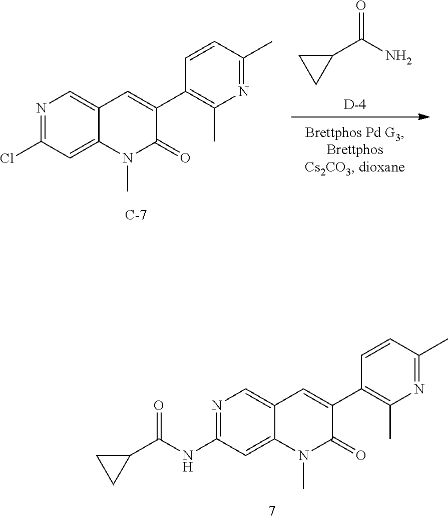

To a solution of 7-chloro-3-(2,6-dimethylpyridin-3-yl)-1-methyl-1,6-naphthyridin-2-one (Compound C-7) (100.0 mg, 0.33 mmol) in dioxane (3.0 mL) was added cyclopropanecarboxamide (Compound D-4) (142.0 mg, 1.67 mmol), BrettPhos (35.8 mg, 0.07 mmol), Cs₂CO₃ (326.1 mg, 1.00 mmol) and BrettPhos Pd G3 (30.2 mg, 0.04 mmol) at room temperature under N₂. The resulting mixture was stirred at 100° C. for 3 h. After the reaction was completed, the resulting mixture was diluted with H₂O and extracted with ethyl acetate. The combined organic layer was washed with brine, dried over anhydrous sodium sulfate, and filtered. The filtrate was concentrated under reduced pressure. The residue was purified by flash column chromatography with petroleum ether/ethyl acetate (80/20, v/v) and then purified by Prep-HPLC with the following conditions (Column: YMC-Actus Triart C18, 30×250 mm, 5 um; Mobile Phase A: Water (10 mmol/L NH₄HCO₃), Mobile Phase B: ACN; Flow rate:50 mL/min; Gradient:37% B to 55% B in 7 min; 254 nm) to afford N-[3-(2,6-dimethylpyridin-3-yl)-1-methyl-2-oxo-1,6-naphthyridin-7-yl]cyclopropanecarboxamide (Compound 7) (35.8 mg, 68%) as a white solid. LCMS (ESI, m/z): [M+H]⁺=349.2. ¹H NMR (300 MHz, DMSO-d₆): δ 11.14 (s, 1H), 8.69 (s, 1H), 8.24 (s, 1H), 7.94 (s, 1H), 7.49 (d, J=8.0 Hz, 1H), 7.13 (d, J=7.6 Hz, 1H), 3.58 (s, 3H), 2.47 (s, 3H), 2.30 (s, 3H), 2.09-2.06 (m, 1H), 0.88-0.85 (m, 4H).

### Example S8. Synthesis of N-(3-(4,6-dimethylpyridin-3-yl)-1-methyl-2-oxo-1,2-dihydro-1,6-naphthyridin-7-yl)cyclopropanecarboxamide (Compound 8)

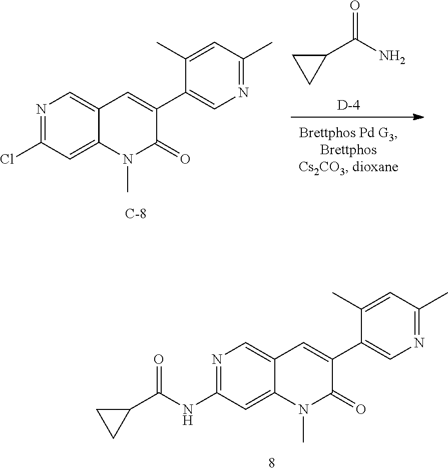

To a solution of 7-chloro-3-(4,6-dimethylpyridin-3-yl)-1-methyl-1,6-naphthyridin-2(1H)-one (Compound C-8) (240.0 mg, 0.80 mmol) in 1,4-dioxane (10.0 mL) was added cyclopropanecarboxamide (Compound D-4) (204.4 mg, 2.40 mmol), BrettPhos (86.0 mg, 0.16 mmol), Cs₂CO₃ (782.6 mg, 2.40 mmol) and BrettPhos Pd G3 (72.6 mg, 0.08 mmol) at room temperature under N₂. The resulting mixture was stirred at 100° C. for 3 h under N₂. After the reaction was completed, the resulting mixture was diluted with H₂O and extracted with ethyl acetate. The combined organic layer was washed with brine, dried over anhydrous sodium sulfate, and filtered. The filtrate was concentrated under vacuum. The residue was purified by flash column chromatography with CH₂Cl₂/CH₃OH (96/4, v/v) and then purified by Prep-HPLC with the following conditions (Column: Xselect CSH OBD Column 30×150 mm, 5 um; Mobile Phase A: Water (0.05% FA), Mobile Phase B:ACN:MeOH=4:1; Flow rate:60 mL/min; Gradient:5% B to 24% B in 8 min; 254 nm) to afford N-(3-(4,6-dimethylpyridin-3-yl)-1-methyl-2-oxo-1,2-dihydro-1,6-naphthyridin-7-yl)cyclopropanecarboxamide (Compound 8) (33.0 mg, 12%) as a white solid. LCMS (ESI, m/z): [M+H]⁺=349.2. ¹H NMR (300 MHz, DMSO-d₆): δ 11.17 (s, 1H), 8.71 (s, 1H), 8.24 (d, J=8.1 Hz, 2H), 7.97 (s, 1H), 7.19 (s, 1H), 3.59 (s, 3H), 2.47 (s, 3H), 2.15 (s, 3H), 2.11-2.05 (m, 1H), 0.89-0.83 (m, 4H).

### Example S9. Synthesis of N-(3-(6-(hydroxymethyl)-4-methylpyridin-3-yl)-1-methyl-2-oxo-1,2-dihydro-1,6-naphthyridin-7-yl)cyclopropanecarboxamide (Compound 9)

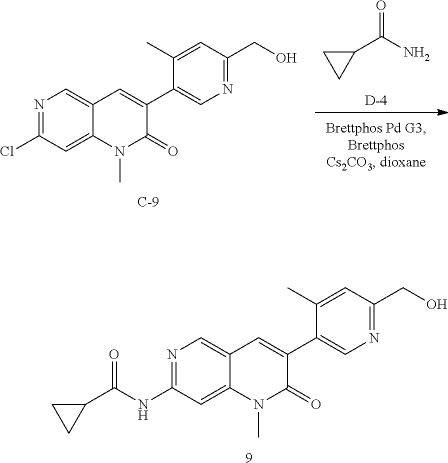

To a solution of 7-chloro-3-(6-(hydroxymethyl)-4-methylpyridin-3-yl)-1-methyl-1,6-naphthyridin-2(1H)-one (Compound C-9) (33.0 mg, 0.11 mmol) in dioxane (2.0 mL) was added cyclopropanecarboxamide (Compound D-4) (53.4 mg, 0.63 mmol), Cs₂CO₃ (102.2 mg, 0.31 mmol), Brettphos (11.2 mg, 0.02 mmol) and BrettPhos Pd G3 (9.5 mg, 0.01 mmol) at room temperature under N₂. The resulting mixture was stirred at 100° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/MeOH (10/1, v/v) and then purified by Prep-HPLC with the following conditions (Column: XBridge Prep OBD C18 Column, 19×250 mm, 5 um; Mobile Phase A: Water (10 mmol/L NH₄HCO₃), Mobile Phase B: MeOH-HPLC; Flow rate: 25 mL/min; Gradient: 40% B to 60% B in 7 min; 254 nm;) to afford N-(3-(6-(hydroxymethyl)-4-methylpyridin-3-yl)-1-methyl-2-oxo-1,2-dihydro-1,6-naphthyridin-7-yl)cyclopropanecarboxamide (Compound 9) (11.3 mg, 29%) as a white solid. LCMS (ESI, m/z): [M+H]⁺=365.1. ¹H NMR (300 MHz, DMSO-d₆): δ 11.16 (s, 1H), 8.72 (s, 1H), 8.27 (s, 1H), 8.25 (s, 1H), 7.99 (s, 1H), 7.39 (s, 1H), 5.44-5.41 (m, 1H), 4.58 (d, J=5.4 Hz, 2H), 3.59 (s, 3H), 2.21 (s, 3H), 2.11-2.08 (m, 1H), 0.89-0.81 (m, 4H).

### Example S10. Synthesis of N-[3-(5-fluoro-4-methylpyridin-3-yl)-2-oxo-1H-1,6-naphthyridin-7-yl]cyclopropanecarboxamide (Compound 10)

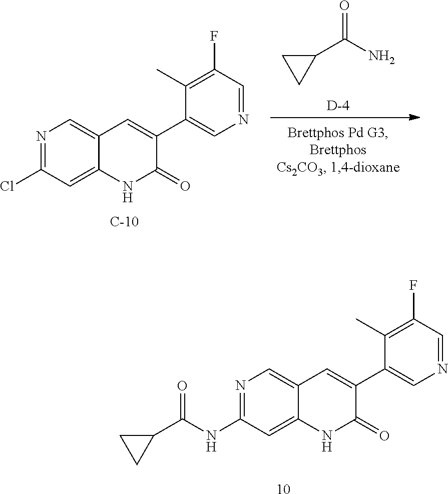

To a solution of 7-chloro-3-(5-fluoro-4-methylpyridin-3-yl)-1H-1,6-naphthyridin-2-one (Compound C-10) (80.0 mg, 0.28 mmol) in 1,4-dioxane (5.0 mL) was added cyclopropanecarboxamide (Compound D-4) (235.0 mg, 2.76 mmol), BrettPhos (29.6 mg, 0.06 mmol), Cs₂CO₃ (269.9 mg, 0.83 mmol) and BrettPhos Pd G3 (25.0 mg, 0.03 mmol). The resulting mixture was irradiated with microwave radiation at 100° C. for 1 h under N₂. After the reaction was completed, the resulting mixture was concentrated under vacuum. The residue was purified by flash column chromatography with petroleum ether/EtOAc (20/80, v/v) and then purified by Prep-HPLC with the following conditions (Column: YMC-Actus Triart C18 Column, 30×250 mm, 5 um; Mobile Phase A: Water (10 mmol/L NH₄HCO₃), Mobile Phase B: ACN; Flow rate: 60 mL/min; Gradient: 15% B to 45% B in 10 min; 254 nm) to afford N-[3-(5-fluoro-4-methylpyridin-3-yl)-2-oxo-1H-1,6-naphthyridin-7-yl]cyclopropanecarboxamide (Compound 10) (23.3 mg, 24%) as a white solid. LCMS (ESI, m/z): [M+H]⁺=339.1. ¹H NMR (300 MHz, DMSO-d₆): δ 12.25 (s, 1H), 11.03 (s, 1H), 8.68 (s, 1H), 8.52 (s, 1H), 8.31 (s, 1H), 8.11 (s, 1H), 8.05 (s, 1H), 2.15 (s, 3H), 2.10-2.02 (m, 1H), 0.87-0.81 (m, 4H).

### Example S11. Synthesis of N-[1-(cyanomethyl)-3-(5-fluoro-4-methylpyridin-3-yl)-2-oxo-1,6-naphthyridin-7-yl]cyclopropanecarboxamide (Compound 11)

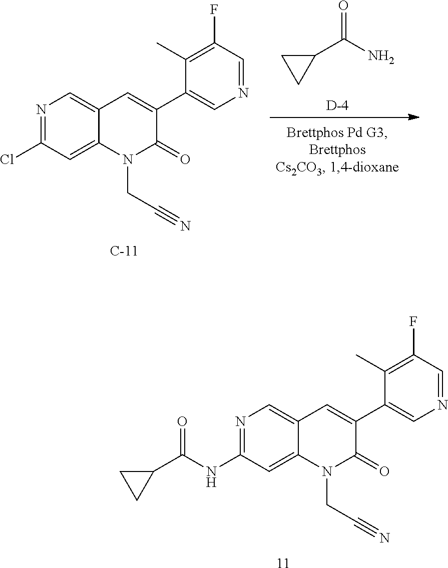

To a solution of 2-[7-chloro-3-(5-fluoro-4-methylpyridin-3-yl)-2-oxo-1,6-naphthyridin-1-yl]acetonitrile (Compound C-11) (120.0 mg, 0.36 mmol) in dioxane (3.0 mL) was added cyclopropanecarboxamide (Compound D-4) (93.2 mg, 1.10 mmol), BrettPhos (78.4 mg, 0.14 mmol), Cs₂CO₃ (356.8 mg, 0.14 mmol) and BrettPhos Pd G3 (66.1 mg, 0.07 mmol) at room temperature under N₂. The resulting mixture was stirred at 100° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/99, v/v) and then purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 18% B to 48% B in 10 min; 254 nm;) to afford N-[1-(cyanomethyl)-3-(5-fluoro-4-methylpyridin-3-yl)-2-oxo-1,6-naphthyridin-7-yl]cyclopropanecarboxamide (Compound 11) (4.8 mg, 4%) as a white solid. LCMS (ESI, m/z): [M+H]⁺=378.2. ¹H NMR (300 MHz, DMSO-d₆): δ 11.29 (s, 1H), 8.80 (s, 1H), 8.55 (s, 1H), 8.35 (s, 1H), 8.34 (s, 1H), 8.18 (s, 1H), 5.36 (s, 2H), 2.15-2.07 (m, 4H), 0.92-0.88 (m, 4H).

### Example S12. Synthesis of (1R,2R)-2-fluoro-N-(1-methyl-3-(4-methylpyridin-3-yl)-2-oxo-1,2-dihydro-1,6-naphthyridin-7-yl)cyclopropane-1-carboxamide (Compound 22)

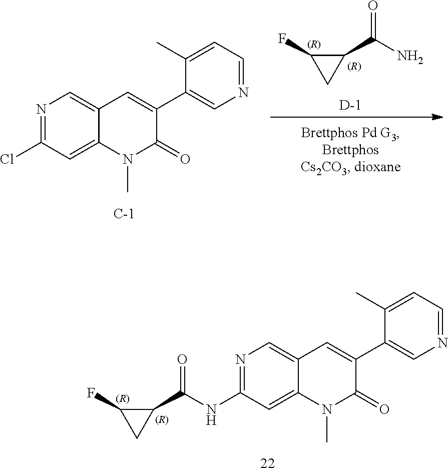

To a solution of 7-chloro-1-methyl-3-(4-methylpyridin-3-yl)-1,6-naphthyridin-2(1H)-one (Compound C-1) (160.0 mg, 0.56 mmol) in 1,4-dioxane (15.0 mL) was added (1R,2R)-2-fluorocyclopropane-1-carboxamide (Compound D-1) (173.2 mg, crude), BrettPhos (60.1 mg, 0.11 mmol), Cs₂CO₃ (547.4 mg, 1.68 mmol) and BrettPhos Pd G3 (50.8 mg, 0.06 mmol) at room temperature under N₂. The resulting mixture was stirred at 100° C. for 3 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (92/8, v/v) to afford (1R,2R)-2-fluoro-N-(1-methyl-3-(4-methylpyridin-3-yl)-2-oxo-1,2-dihydro-1,6-naphthyridin-7-yl)cyclopropane-1-carboxamide (Compound 22) (35.2 mg, 18%) as a white solid. LCMS (ESI, m/z): [M+H]⁺=353.2. ¹H NMR (400 MHz, DMSO-d₆): δ 11.22 (s, 1H), 8.72 (s, 1H), 8.46 (d, J=5.2 Hz, 1H), 8.37 (s, 1H), 8.25 (s, 1H), 8.03 (s, 1H), 7.33 (d, J=4.8 Hz, 1H), 5.08-4.88 (m, 1H), 3.61 (s, 3H), 2.32-2.26 (m, 1H), 2.20 (s, 3H), 1.75-1.66 (m, 1H), 1.28-1.19 (m, 1H).

### Example S13. Synthesis of (1S,2S)-2-fluoro-N-(1-methyl-3-(4-methylpyridin-3-yl)-2-oxo-1,2-dihydro-1,6-naphthyridin-7-yl)cyclopropane-1-carboxamide (Compound 23)

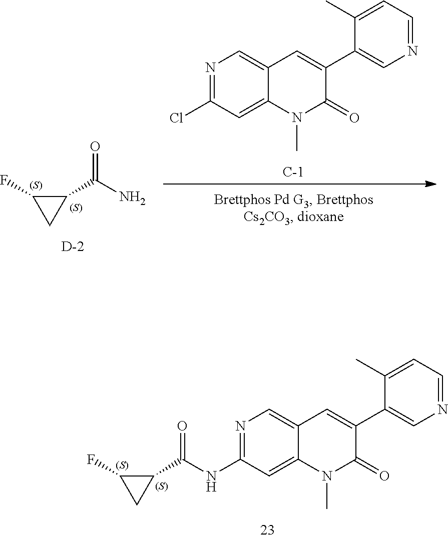

To a solution of 7-chloro-1-methyl-3-(4-methylpyridin-3-yl)-1,6-naphthyridin-2(1H)-one (Compound C-1) 170.0 mg, 0.59 mmol) in 1,4-dioxane (3.0 mL) was added (1S,2S)-2-fluorocyclopropane-1-carboxamide (Compound D-2) (184.0 mg, crude), BrettPhos (63.9 mg, 0.12 mmol), Cs₂CO₃ (581.6 mg, 1.79 mmol) and BrettPhos Pd G3 (53.9 mg, 0.06 mmol) at room temperature under N₂. The resulting mixture was stirred at 100° C. for 3 h under N₂. After the reaction was completed, the resulting mixture was diluted with H₂O and extracted with ethyl acetate. The combined organic layer was washed with brine, dried over anhydrous sodium sulfate, and filtered. The filtrate was concentrated under vacuum. The residue was purified by flash column chromatography with CH₂Cl₂/CH₃OH (10/1, v/v) to afford (1S,2S)-2-fluoro-N-(1-methyl-3-(4-methylpyridin-3-yl)-2-oxo-1,2-dihydro-1,6-naphthyridin-7-yl)cyclopropane-1-carboxamide (Compound 23) (39.1 mg, 18%) as a white solid. LCMS (ESI, m/z): [M+H]⁺=353.1. ¹H NMR (400 MHz, DMSO-d₆): δ 11.22 (s, 1H), 8.72 (s, 1H), 8.46 (d, J=4.8 Hz, 1H), 8.37 (s, 1H), 8.25 (s, 1H), 8.03 (s, 1H), 7.33 (d, J=5.2 Hz, 1H), 5.07-4.89 (m, 1H), 3.61 (s, 3H), 2.31-2.27 (m, 1H), 2.20 (s, 3H), 1.73-1.66 (m, 1H), 1.26-1.23 (m, 1H).

### Example S14. Synthesis of (1R,2R)-2-fluoro-N-[3-(5-fluoro-4-methylpyridin-3-yl)-1-methyl-2-oxo-1,6-naphthyridin-7-yl]cyclopropane-1-carboxamide (Compound 27)

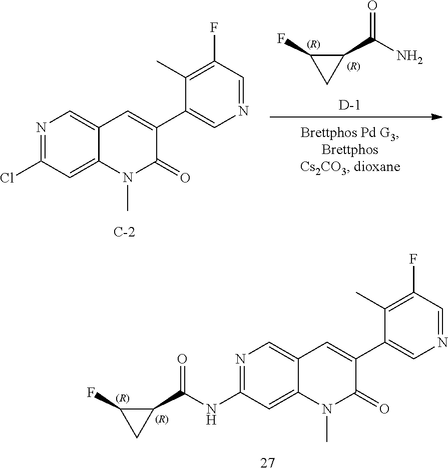

To a solution of 7-chloro-3-(5-fluoro-4-methylpyridin-3-yl)-1-methyl-1,6-naphthyridin-2-one (Compound C-2) (100.0 mg, 0.33 mmol) in 1,4-dioxane (4.0 mL) was added (1R,2R)-2-fluorocyclopropane-1-carboxamide (Compound D-1) (169.7 mg, 1.65 mmol), BrettPhos (35.3 mg, 0.07 mmol), Cs₂CO₃ (321.3 mg, 0.99 mmol) and BrettPhos Pd G3 (29.9 mg, 0.03 mmol) at room temperature under N₂. The resulting mixture was stirred at 100° C. for 3 h under N₂. After the reaction was completed, the resulting mixture was diluted with H₂O and extracted with ethyl acetate. The combined organic layer was washed with brine, dried over anhydrous sodium sulfate, and filtered. The filtrate was concentrated under reduced pressure. The residue was purified by Prep-HPLC with the following conditions (Column: XBridge Prep OBD C18 Column, 19×250 mm, 5 um; Mobile Phase A: Water (0.05% NH₃·H₂O), Mobile Phase B: ACN; Flow rate:25 mL/min; Gradient:54% B to 72% B in 10 min; 254 nm) to afford (1R,2R)-2-fluoro-N-[3-(5-fluoro-4-methylpyridin-3-yl)-1-methyl-2-oxo-1,6-naphthyridin-7-yl]cyclopropane-1-carboxamide (Compound 27) (12.1 mg, 10%) as a white solid. LCMS (ESI, m/z): [M+H]⁺=371.2. ¹H NMR (300 MHz, DMSO-d₆): δ 11.25 (s, 1H), 8.74 (s, 1H), 8.54 (s, 1H), 8.31 (s, 1H), 8.27 (s, 1H), 8.09 (s, 1H), 5.17-4.71 (m, 1H), 3.62 (s, 3H), 2.39-2.22 (m, 1H), 2.14 (s, 3H), 1.76-1.67 (m, 1H), 1.32-1.18 (m, 1H).

### Example S15. Synthesis of (1S,2S)-2-fluoro-N-[3-(5-fluoro-4-methylpyridin-3-yl)-1-methyl-2-oxo-1,6-naphthyridin-7-yl]cyclopropane-1-carboxamide (Compound 28)

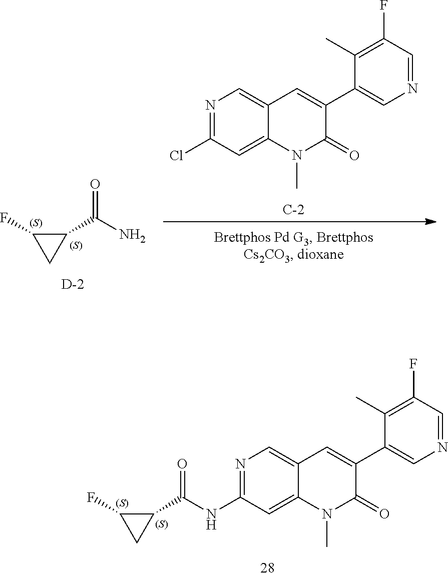

To a stirred solution of 7-chloro-3-(5-fluoro-4-methylpyridin-3-yl)-1-methyl-1,6-naphthyridin-2-one (Compound C-2) (100.0 mg, 0.33 mmol) in 1,4-dioxane (4.0 mL) was added (1S,2S)-2-fluorocyclopropane-1-carboxamide (Compound D-2) (169.7 mg, 1.65 mmol), BrettPhos (35.3 mg, 0.07 mmol), Cs₂CO₃ (321.3 mg, 0.99 mmol) and BrettPhos Pd G3 (29.9 mg, 0.03 mmol) at room temperature under N₂. The resulting mixture was stirred at 100° C. for 3 h under N₂. After the reaction was completed, the resulting mixture was diluted with H₂O and extracted with ethyl acetate. The combined organic layer was washed with brine, dried over anhydrous sodium sulfate, and filtered. The filtrate was concentrated under vacuum. The residue was purified by Prep-HPLC with the following conditions (Column: XBridge Prep OBD C18 Column, 19×250 mm, 5 um; Mobile Phase A: Water (0.05% NH₃H₂O), Mobile Phase B: ACN; Flow rate:25 mL/min; Gradient:54% B to 72% B in 10 min; 254 nm) to afford (1S,2S)-2-fluoro-N-[3-(5-fluoro-4-methylpyridin-3-yl)-1-methyl-2-oxo-1,6-naphthyridin-7-yl]cyclopropane-1-carboxamide (Compound 28) (11.2 mg, 9%) as a white solid. LCMS (ESI, m/z): [M+H]⁺=371.2. ¹H NMR (400 MHz, DMSO-d₆): δ 11.24 (s, 1H), 8.74 (s, 1H), 8.54 (s, 1H), 8.31 (s, 1H), 8.27 (s, 1H), 8.10 (d, J=6.4 Hz, 1H), 5.07-4.90 (m, 1H), 3.62 (s, 3H), 2.34-2.28 (m, 1H), 2.14 (s, 3H), 1.76-1.67 (m, 1H), 1.32-1.18 (m, 1H).

### Example S16. Synthesis of (1S,2S)-2-fluoro-N-[3-(4-methylpyridin-3-yl)-2-oxo-1H-1,6-naphthyridin-7-yl]cyclopropane-1-carboxamide (Compound 33)

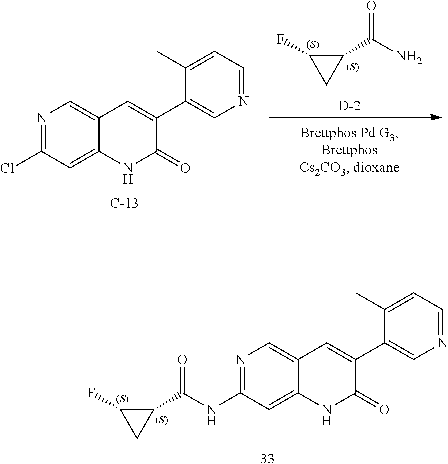

To a solution of 7-chloro-3-(4-methylpyridin-3-yl)-1H-1,6-naphthyridin-2-one (Compound C-13) (130.0 mg, 0.48 mmol) in 1,4-dioxane (5.0 mL) was added (1S,2S)-2-fluorocyclopropane-1-carboxamide (Compound D-2) (246.6 mg, 2.39 mmol), BrettPhos (51.4 mg, 0.10 mmol), Cs₂CO₃ (467.7 mg, 1.44 mmol) and BrettPhos Pd G3 (43.4 mg, 0.05 mmol). The resulting mixture was stirred at 100° C. for 2 h under N₂. After the reaction was completed, the resulting mixture was concentrated under vacuum. The residue was purified by flash column chromatography with CH₂Cl₂/MeOH (95/5, v/v) and then purified by Prep-HPLC with the following conditions (Column: Xcelect CSH F-pheny OBD Column, 19×250 mm, 5 um; Mobile Phase A: Water (10 mmol/L NH₄HCO₃), Mobile Phase B: ACN; Flow rate: 60 mL/min; Gradient: 45% B to 48% B in 11 min; 254 nm) to afford (1S,2S)-2-fluoro-N-[3-(4-methylpyridin-3-yl)-2-oxo-1H-1,6-naphthyridin-7-yl]cyclopropane-1-carboxamide (Compound 33) (13.0 mg, 8%) as a white solid. LCMS (ESI, m/z): [M+H]⁺=339.2. ¹H NMR (300 MHz, DMSO-d₆): δ 12.21 (s, 1H), 11.05 (s, 1H), 8.68 (s, 1H), 8.45 (d, J=4.8 Hz, 1H), 8.38 (s, 1H), 8.11 (s, 1H), 8.00 (s, 1H), 7.32 (d, J=5.1 Hz, 1H), 5.10-4.82 (m, 1H), 2.28-2.22 (m, 4H), 1.75-1.64 (m, 1H), 1.27-1.15 (m, 1H).

### Example S17. Synthesis of (1S,2S)—N-[1-ethyl-3-(5-fluoro-4-methylpyridin-3-yl)-2-oxo-1,6-naphthyridin-7-yl]-2-fluorocyclopropane-1-carboxamide (Compound 48)

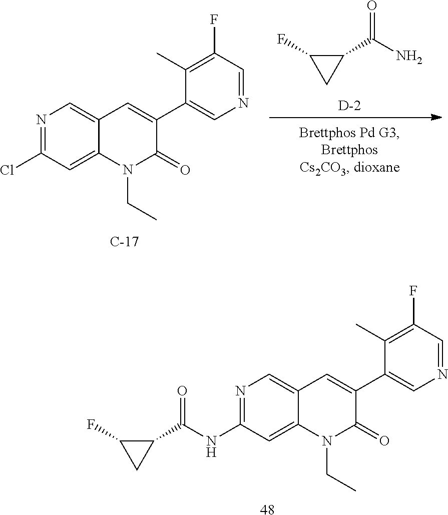

To a solution of 7-chloro-1-ethyl-3-(5-fluoro-4-methylpyridin-3-yl)-1,6-naphthyridin-2-one (Compound C-17) (150.0 mg, 0.47 mmol) in 1,4-dioxane (5.0 mL) was added (1S,2S)-2-fluorocyclopropane-1-carboxamide (Compound D-2) (243.3 mg, 2.36 mmol), BrettPhos (50.7 mg, 0.09 mmol), Cs₂CO₃ (461.4 mg, 1.42 mmol) and BrettPhos Pd G3 (42.8 mg, 0.05 mmol). The resulting mixture was irradiated with microwave radiation at 100° C. for 3 h under N₂. After the reaction was completed, the resulting mixture was concentrated under vacuum. The residue was purified by flash column chromatography with petroleum ether/EtOAc (40/60, v/v) and then purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 15% B to 45% B in 10 min; 254 nm) to afford (1S,2S)—N-[1-ethyl-3-(5-fluoro-4-methylpyridin-3-yl)-2-oxo-1,6-naphthyridin-7-yl]-2-fluorocyclopropane-1-carboxamide (Compound 48) (5.2 mg, 3%) as a white solid. LCMS (ESI, m/z): [M+H]⁺=385.2. ¹H NMR (400 MHz, DMSO-d₆): δ 11.22 (s, 1H), 8.75 (s, 1H), 8.53 (s, 1H), 8.32 (s, 1H), 8.30 (s, 1H), 8.09 (s, 1H), 5.07-4.89 (m, 1H), 4.24-4.21 (m, 2H), 2.30-2.27 (m, 1H), 2.13 (d, J=1.6 Hz, 3H), 1.75-1.67 (m, 1H), 1.36-1.14 (m, 4H).

### Example S18. Synthesis of 2,2-difluoro-N-(1-methyl-3-(4-methylpyridin-3-yl)-2-oxo-1,2-dihydro-1,6-naphthyridin-7-yl)cyclopropane-1-carboxamide (Compound 61)

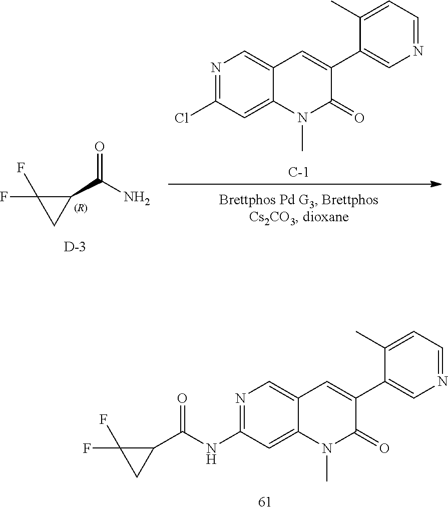

To a solution of 7-chloro-1-methyl-3-(4-methylpyridin-3-yl)-1,6-naphthyridin-2(1H)-one (Compound C-1) (140.0 mg, 0.49 mmol) in 1,4-dioxane (10.0 mL) was added (R)-2,2-difluorocyclopropane-1-carboxamide (Compound D-3) (71.2 mg, 0.58 mmol), BrettPhos (52.6 mg, 0.10 mmol), Cs₂CO₃ (478.9 mg, 1.47 mmol) and BrettPhos Pd G3 (44.4 mg, 0.05 mmol) at room temperature under N₂. The resulting mixture was stirred at 100° C. for 3 h under N₂. After the reaction was completed, the resulting mixture was diluted with H₂O and extracted with ethyl acetate. The combined organic layer was washed with brine, dried over anhydrous sodium sulfate, and filtered. The filtrate was concentrated under vacuum. The residue was purified by flash column chromatography with CH₂Cl₂/CH₃OH (92/8, v/v) to afford 2,2-difluoro-N-(1-methyl-3-(4-methylpyridin-3-yl)-2-oxo-1,2-dihydro-1,6-naphthyridin-7-yl)cyclopropane-1-carboxamide (Compound 61) (52.8 mg, 29%) as an off-white solid. LCMS (ESI, m/z): [M+H]⁺=371.1. ¹H NMR (300 MHz, DMSO-d₆): δ 11.38 (s, 1H), 8.75 (s, 1H), 8.47 (d, J=5.1 Hz, 1H), 8.38 (s, 1H), 8.22 (s, 1H), 8.04 (s, 1H), 7.34 (d, J=4.8 Hz, 1H), 3.62 (s, 3H), 3.13-3.02 (m, 1H), 2.20 (s, 3H), 2.13-2.04 (m, 2H).

### Example S19. Synthesis of N-(3-[6-[(1S)-1-hydroxypropyl]-4-methylpyridin-3-yl]-1-methyl-2-oxo-1,6-naphthyridin-7-yl)cyclopropanecarboxamide (Compound 69)

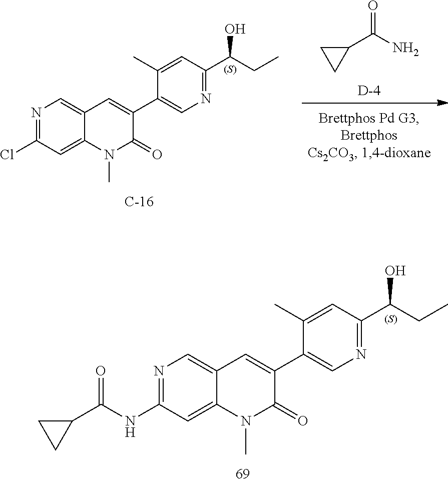

A mixture of 7-chloro-3-[6-[(1S)-1-hydroxypropyl]-4-methylpyridin-3-yl]-1-methyl-1,6-naphthyridin-2-one (Compound C-16) (300.0 mg, 0.87 mmol), cyclopropanecarboxamide (Compound D-4) (222.8 mg, 2.62 mmol), Cs₂CO₃ (852.9 mg, 2.62 mmol), BrettPhos (93.7 mg, 0.17 mmol) and BrettPhos Pd G3 (79.1 mg, 0.09 mmol) in dioxane (15.0 mL) was stirred at 100° C. for 2 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (94/6, v/v) and then purified by Prep-HPLC with the following conditions (Column: Xselect CSH OBD Column 30×50 mm, 5 um; Mobile Phase A: Water (0.1% FA), Mobile Phase B: ACN; Flow rate:60 mL/min; Gradient: 7% B to 22% B in 10 min; 254/220 nm) to afford the desired product. The product was separated by chiral-HPLC with the following conditions: [Column: CHIRALPAK IA, 2×25 cm, 5 um; Mobile Phase A:Hex:DCM=3:1 (0.5% 2M NH₃-MeOH)-HPLC, Mobile Phase B: EtOH-HPLC; Flow rate: 18 mL/min; Gradient:10% B to 10% B in 22 min; 220/254 nm; RT1:14.777 min; RT2:18.58 min; Injection Volume:0.8 ml] to afford N-(3-[6-[(1S)-1-hydroxypropyl]-4-methylpyridin-3-yl]-1-methyl-2-oxo-1,6-naphthyridin-7-yl)cyclopropanecarboxamide (Compound 69) (13.8 mg, 4%) as a white solid. LCMS (ESI, m/z): [M+H]⁺=393.2. ¹H NMR (300 MHz, DMSO-d₆): δ 11.13 (s, 1H), 8.70 (s, 1H), 8.26 (s, 1H), 8.24 (s, 1H), 7.99 (s, 1H), 7.39 (s, 1H), 5.28 (d, J=4.8 Hz, 1H), 4.52-4.48 (m, 1H), 3.59 (s, 3H), 2.28-2.07 (m, 4H), 1.84-1.73 (m, 1H), 1.71-1.52 (m, 1H), 1.03-0.79 (m, 7H).

### Example S20. Synthesis of N-(1-methyl-3-(4-methyl-6-(2,2,2-trifluoro-1-hydroxyethyl)pyridin-3-yl)-2-oxo-1,2-dihydro-1,6-naphthyridin-7-yl)cyclopropanecarboxamide (Compound 72 and 76)

### Step 1: Synthesis of (1S,2S)—N-[3-(6-[1-((tert-butyldimethylsilyl)oxy]-2,2,2-trifluoroethyl]-4-methylpyridin-3-yl)-1-methyl-2-oxo-1,6-naphthyridin-7-yl]-2-fluorocyclopropane-1-carboxamide (E-72 and E-76)

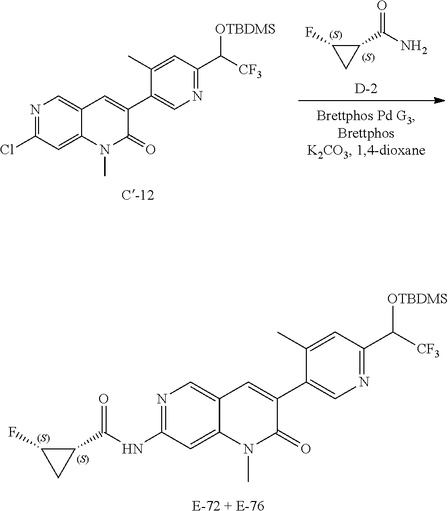

To a solution of 3-(6-[1-[(tert-butyldimethylsilyl)oxy]-2,2,2-trifluoroethyl]-4-methylpyridin-3-yl)-7-chloro-1-methyl-1,6-naphthyridin-2-one (Compound C′-12) (500.0 mg, 1.00 mmol) in 1,4-dioxane (8.0 mL) was added (1S,2S)-2-fluorocyclopropane-1-carboxamide (Compound D-2) (517.5 mg, 5.02 mmol), K₂CO₃ (277.5 mg, 2.01 mmol), BrettPhos (107.8 mg, 0.20 mmol) and BrettPhos Pd G3 (91.0 mg, 0.10 mmol) at room temperature under N₂. The resulting mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/3, v/v) to afford (1S,2S)—N-[3-(6-[1-[(tert-butyldimethylsilyl)oxy]-2,2,2-trifluoroethyl]-4-methylpyridin-3-yl)-1-methyl-2-oxo-1,6-naphthyridin-7-yl]-2-fluorocyclopropane-1-carboxamide (mixture of Compounds E-72 and E-76) (220.0 mg, 38%) as a white solid. LCMS (ESI, m/z): [M+H]⁺=565.2.

### Step 2: Synthesis of (1S,2S)-2-fluoro-N-(1-methyl-3-(4-methyl-6-(2,2,2-tri fluoro-1-hydroxyethyl)pyridin-3-yl)-2-oxo-1,2-dihydro-1,6-naphthyridin-7-yl)cyclopropanecarboxamide (Compounds 72 and 76)

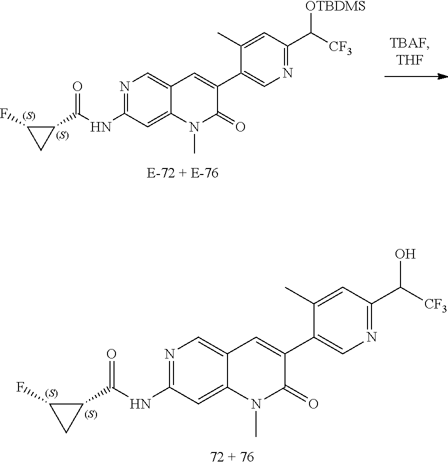

To a solution of (1S,2S)—N-[3-(6-[1-[(tert-butyldimethylsilyl)oxy]-2,2,2-trifluoroethyl]-4-methylpyridin-3-yl)-1-methyl-2-oxo-1,6-naphthyridin-7-yl]-2-fluorocyclopropane-1-carboxamide (Compounds E-72 and E-76) (240.0 mg, 0.43 mmol) in THE (10.0 mL) was added TBAF (0.85 mL, 0.85 mmol) at room temperature. The resulting mixture was stirred at room temperature for 16 h. After the reaction was completed, the resulting mixture was concentrated under reduced pressure. The residue was purified by reverse phase flash chromatography with CH₃CN/H₂O (1/2, v/v) to afford (1S,2S)-2-fluoro-N-(1-methyl-3-(4-methyl-6-(2,2,2-trifluoro-1-hydroxyethyl)pyridin-3-yl)-2-oxo-1,2-dihydro-1,6-naphthyridin-7-yl)cyclopropanecarboxamide (Compounds 72 and 76) (110.0 mg, 57%) as a light yellow solid. LCMS (ESI, m/z): [M+H]⁺=451.1.

### Example S21. Chiral Separation of (1S,2S)-2-fluoro-N-(1-methyl-3-[4-methyl-6-[(1S)-2,2,2-trifluoro-1-hydroxyethyl]pyridin-3-yl]-2-oxo-1,6-naphthyridin-7-yl)cyclopropane-1-carboxamide and (1S,2S)-2-fluoro-N-(1-methyl-3-[4-methyl-6-[(1R)-2,2,2-trifluoro-1-hydroxyethyl]pyridin-3-yl]-2-oxo-1,6-naphthyridin-7-yl)cyclopropane-1-carboxamide (Compound 72 and 76)

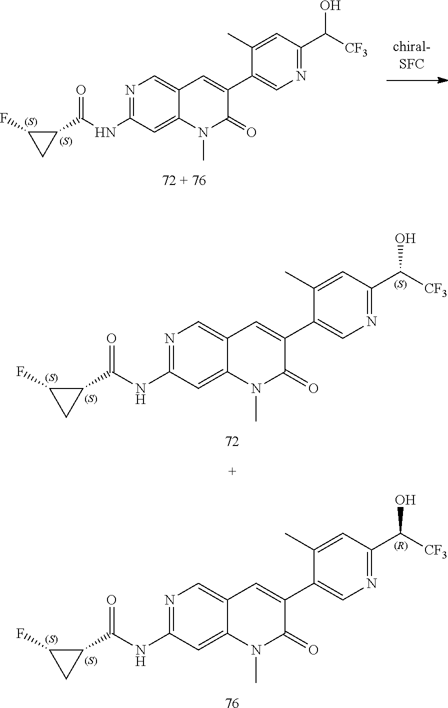

The (1S,2S)-2-fluoro-N-(1-methyl-3-(4-methyl-6-(2,2,2-trifluoro-1-hydroxyethyl)pyridin-3-yl)-2-oxo-1,2-dihydro-1,6-naphthyridin-7-yl)cyclopropanecarboxamide (mixture of Compounds 72 and 76) (110.0 mg, 0.24 mmol) was separated by Prep-Chiral-HPLC with the following conditions (Column: CHIRALPAK IA, 2×25 cm, 5 um; Mobile Phase A: Hex (0.1% FA)-HPLC, Mobile Phase B: EtOH-HPLC; Flow rate: 20 mL/min; Gradient: 40% B to 40% B in 23 min; 220/254 nm) to afford Enantiomer A (43.0 mg, 22%, retention time=20.261 min) as a light yellow solid, and Enantiomer B (48.8 mg, 25%, retention time=15.289 min) as a light yellow solid.

Enantiomer A: LCMS (ESI, m/z): [M+H]⁺=451.2. ¹H NMR (300 MHz, CD₃CN): δ 11.22 (s, 1H), 8.71 (s, 1H), 8.42 (s, 1H), 8.25 (s, 1H), 8.07 (s, 1H), 7.55 (s, 1H), 7.05 (d, J=5.4 Hz, 1H), 5.14-4.84 (m, 2H), 3.61 (s, 3H), 2.29-2.24 (m, 4H), 1.76-1.63 (m, 1H), 1.29-1.09 (m, 1H).

Enantiomer B: LCMS (ESI, m/z): [M+H]⁺=451.2. ¹H NMR (300 MHz, CD₃CN): δ 11.22 (s, 1H), 8.71 (s, 1H), 8.37 (s, 1H), 8.25 (s, 1H), 8.07 (s, 1H), 7.55 (s, 1H), 7.06 (s, 1H), 5.15-4.84 (m, 2H), 3.61 (s, 3H), 2.33-2.19 (m, 4H), 1.76-1.63 (m, 1H), 1.28-1.17 (m, 1H).

The absolute stereochemistry of enantiomers A and B was not assigned. The two enantiomeric structures that could be obtained from chiral separation of the enantiomeric mixture as described above are shown as Compounds 72 and 76 in Table 1.

### Example S22. Synthesis of N-(1-methyl-3-(4-methyl-6-(2,2,2-trifluoro-1-hydroxyethyl)pyridin-3-yl)-2-oxo-1,2-dihydro-1,6-naphthyridin-7-yl)cyclopropanecarboxamide (Compound 79)

### Step 1: Synthesis of N-(3-(6-(1-((tert-butyldimethylsilyl)oxy)-2,2,2-trifluoroethyl)-4-methylpyridin-3-yl)-1-methyl-2-oxo-1,2-dihydro-1,6-naphthyridin-7-yl)cyclopropanecarboxamide (E-79)

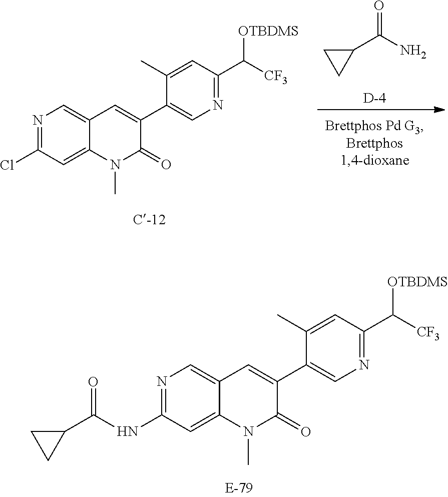

To a solution of 3-(6-(1-((tert-butyldimethylsilyl)oxy)-2,2,2-trifluoroethyl)-4-methylpyridin-3-yl)-7-chloro-1-methyl-1,6-naphthyridin-2(1H)-one (Compound C′-12) (300.0 mg, 0.60 mmol) in 1,4-dioxane (5.0 mL) was added cyclopropanecarboxamide (Compound D-4) (256.3 mg, 3.01 mmol), K₂CO₃ (249.8 mg, 1.81 mmol), BrettPhos (64.7 mg, 0.12 mmol) and BrettPhos Pd G3 (54.6 mg, 0.06 mmol) at room temperature under N₂. The resulting mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (50/50, v/v) to afford N-(3-(6-(1-((tert-butyldimethylsilyl)oxy)-2,2,2-trifluoroethyl)-4-methylpyridin-3-yl)-1-methyl-2-oxo-1,2-dihydro-1,6-naphthyridin-7-yl)cyclopropanecarboxamide (Compound E-79) (60.0 mg, 15%) as a white solid. LCMS (ESI, m/z): [M+H]⁺=547.2.

### Step 2: Synthesis of N-(1-methyl-3-(4-methyl-6-(2,2,2-tri fluoro-1-hydroxyethyl)pyridin-3-yl)-2-oxo-1,2-dihydro-1,6-naphthyridin-7-yl)cyclopropanecarboxamide (79)

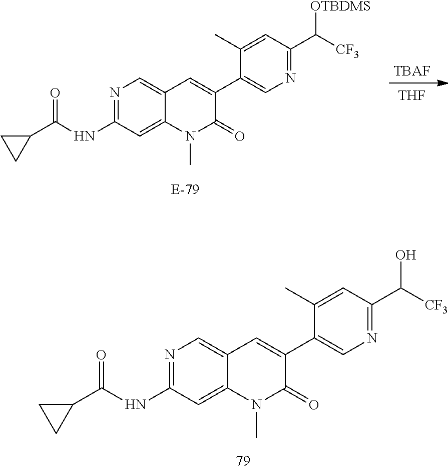

To a solution of N-(3-(6-(1-((tert-butyldimethylsilyl)oxy)-2,2,2-trifluoroethyl)-4-methylpyridin-3-yl)-1-methyl-2-oxo-1,2-dihydro-1,6-naphthyridin-7-yl)cyclopropanecarboxamide (Compound E-79) (60.0 mg, 0.11 mmol) in THE (5.0 mL) was added TBAF (0.3 mL, 0.22 mmol) at 0° C. under N₂. The resulting mixture was stirred at 0° C. for 0.5 h. After the reaction was completed, the resulting mixture was concentrated under reduced pressure. The residue was purified by reverse phase flash chromatography with CH₃CN/H₂O (16/84, v/v) to afford N-(1-methyl-3-(4-methyl-6-(2,2,2-trifluoro-1-hydroxyethyl)pyridin-3-yl)-2-oxo-1,2-dihydro-1,6-naphthyridin-7-yl)cyclopropanecarboxamide (Compound 79) (11.9 mg, 25%) as a light yellow solid. LCMS (ESI, m/z): [M+H]⁺=433.1. ¹H NMR (300 MHz, DMSO-d₆): δ 11.18 (s, 1H), 8.71 (s, 1H), 8.38 (s, 1H), 8.26 (s, 1H), 8.07 (s, 1H), 7.56 (s, 1H), 7.10 (s, 1H), 5.15-5.13 (m, 1H), 3.60 (s, 3H), 2.25 (s, 3H), 2.14-2.05 (m, 1H), 0.90-0.86 (m, 4H).

### Example S23. Chiral Separation of (S)—N-(1-methyl-3-(4-methyl-6-(2,2,2-trifluoro-1-hydroxyethyl)pyridin-3-yl)-2-oxo-1,2-dihydro-1,6-naphthyridin-7-yl)cyclopropanecarboxamide and (R)—N-(1-methyl-3-(4-methyl-6-(2,2,2-trifluoro-1-hydroxyethyl)pyridin-3-yl)-2-oxo-1,2-dihydro-1,6-naphthyridin-7-yl)cyclopropanecarboxamide (Compound 80 and 81)

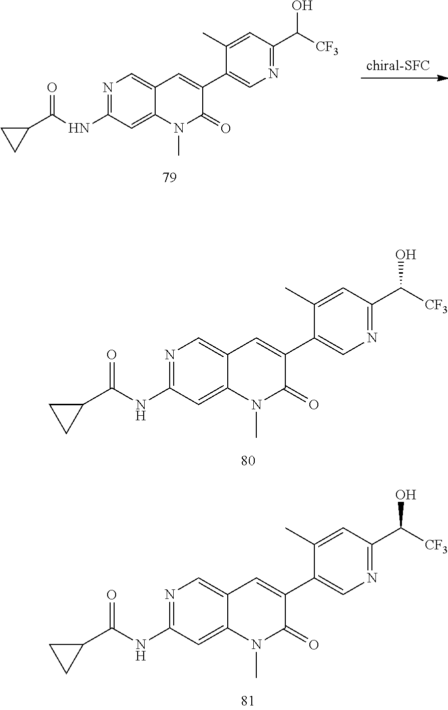 [SMILES: Cc1cc(C(O)C(F)(F)F)ncc1-c1cc2cnc(NC(=O)C3CC3)cc2n(C)c1=O ; "chiral-SFC" ; Cc1cc([C@H](O)C(F)(F)F)ncc1-c1cc2cnc(NC(=O)C3CC3)cc2n(C)c1=O ; Cc1cc([C@@H](O)C(F)(F)F)ncc1-c1cc2cnc(NC(=O)C3CC3)cc2n(C)c1=O]

The N-(1-methyl-3-(4-methyl-6-(2,2,2-trifluoro-1-hydroxyethyl)pyridin-3-yl)-2-oxo-1,2-dihydro-1,6-naphthyridin-7-yl)cyclopropanecarboxamide (Compound 79) (52.0 mg, 0.10 mmol) was separated by Prep-Chiral-HPLC with the following conditions (Column: CHIRALPAK IA, 2×25 cm, 5 um; Mobile Phase A: Hex (8 mmol/L NH₃·MeOH)-HPLC, Mobile Phase B: EtOH-HPLC; Flow rate: 20 mL/min; Gradient: 30% B to 30% B in 29 min; 254 nm) to afford Enantiomer 79A (5.2 mg, 12%, elution time=18.424 min) as a white solid and Enantiomer 79B (4.5 mg, 10%, elution time=23.962 min) as a white solid.

Enantiomer 79A: LCMS (ESI, m/z): [M+H]⁺=433.2. ¹H NMR (300 MHz, CD₃CN) δ 9.18 (s, 1H), 8.58 (s, 1H), 8.43 (s, 1H), 8.31 (s, 1H), 7.85 (s, 1H), 7.51 (s, 1H), 5.22-5.13 (m, 2H), 3.65 (s, 3H), 2.31 (s, 3H), 1.93-1.86 (m, 1H), 1.04-0.98 (m, 2H), 0.97-0.86 (m, 2H).

Enantiomer 79B: LCMS (ESI, m/z): [M+H]⁺=433.2. ¹H NMR (300 MHz, CD₃CN) δ 9.18 (s, 1H), 8.58 (s, 1H), 8.43 (s, 1H), 8.31 (s, 1H), 7.85 (s, 1H), 7.51 (s, 1H), 5.20-5.15 (m, 2H), 3.65 (s, 3H), 2.31 (s, 3H), 1.93-1.86 (m, 1H), 1.04-0.98 (m, 2H), 0.97-0.86 (m, 2H).

The absolute stereochemistry of enantiomers 79A and 79B was not assigned. The two enantiomeric structures that could be obtained from chiral separation of the enantiomeric mixture of Compound 79 as described above are shown as Compounds 80 and 81 in Table 1.

### Example S24. Synthesis of 2,2-difluoro-N-(3-(5-fluoro-4-methylpyridin-3-yl)-1-methyl-2-oxo-1,2-dihydro-1,6-naphthyridin-7-yl)cyclopropanecarboxamide (Compound 82)

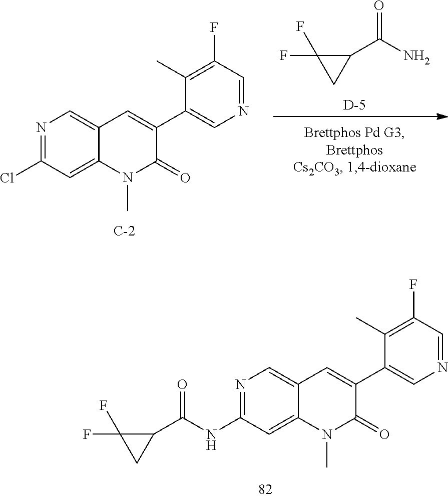

To a solution of 7-chloro-3-(5-fluoro-4-methylpyridin-3-yl)-1-methyl-1,6-naphthyridin-2-one (Compound C-2) (250.0 mg, 0.82 mmol) in 1,4-dioxane (10.0 mL) was added 2,2-difluorocyclopropane-1-carboxamide (Compound D-5) (498.4 mg, 4.12 mmol), BrettPhos (88.4 mg, 0.17 mmol), Cs₂CO₃ (804.6 mg, 2.47 mmol) and BrettPhos Pd G3 (74.6 mg, 0.08 mmol) at room temperature under N₂. The resulting mixture was irradiated with microwave radiation at 120° C. for 1.5 h. After the reaction was completed, the resulting mixture was diluted with H₂O and extracted with DCM. The combined organic layer was washed with brine, dried over anhydrous sodium sulfate and filtered. The filtrate was concentrated under reduced pressure. The residue was purified by flash column chromatography with petroleum ether/ethyl acetate (1/2, v/v) to afford 2,2-difluoro-N-(3-(5-fluoro-4-methylpyridin-3-yl)-1-methyl-2-oxo-1,2-dihydro-1,6-naphthyridin-7-yl)cyclopropanecarboxamide (Compound 82) (17.1 mg, 5%) as a white solid. LCMS (ESI, m/z): [M+H]⁺=389.2. ¹H NMR (300 MHz, DMSO-d₆): δ 11.37 (s, 1H), 8.75 (s, 1H), 8.53 (s, 1H), 8.30 (s, 1H), 8.22 (s, 1H), 8.09 (s, 1H), 3.61 (s, 3H), 3.09-3.05 (m, 1H), 2.13-2.03 (m, 5H).

### Example S25. Synthesis of (1S,2S)-2-fluoro-N-(3-[6-[(1R)-1-hydroxyethyl]-4-methylpyridin-3-yl]-1-methyl-2-oxo-1,6-naphthyridin-7-yl)cyclopropane-1-carboxamide (Compound 86)

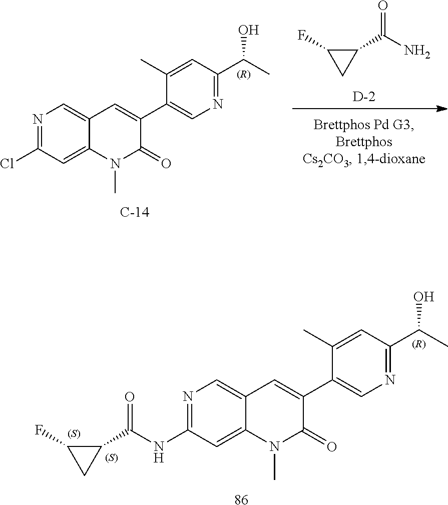

A mixture of 7-chloro-3-[6-[(1R)-1-hydroxyethyl]-4-methylpyridin-3-yl]-1-methyl-1,6-naphthyridin-2-one (Compound C-14) (200.0 mg, 0.61 mmol), (1S,2S)-2-fluorocyclopropane-1-carboxamide (Compound D-2) (312.6 mg, 3.03 mmol), Cs₂CO₃ (592.8 mg, 1.82 mmol), BrettPhos (65.1 mg, 0.12 mmol) and BrettPhos Pd G3 (55.0 mg, 0.06 mmol) in dioxane (2.0 mL) was irradiated with microwave radiation at 120° C. for 1 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (94/6, v/v) and then purified by Prep-HPLC with the following conditions (Column: YMC-Actus Triart C18, 20×250 mm, 5 um, 12 nm; Mobile Phase A: Water (10 mmol/L NH₄HCO₃), Mobile Phase B: ACN; Flow rate: 60 mL/min; Gradient: 13% B to 43% B in 10 min; 254 nm) to afford (1S,2S)-2-fluoro-N-(3-[6-[(1R)-1-hydroxyethyl]-4-methylpyridin-3-yl]-1-methyl-2-oxo-1,6-naphthyridin-7-yl)cyclopropane-1-carboxamide (Compound 86) (17.5 mg, 7%) as a white solid. LCMS (ESI, m/z): [M+H]⁺=397.2. ¹H NMR (300 MHz, DMSO-d₆): δ 11.19 (s, 1H), 8.71 (s, 1H), 8.26 (s, 1H), 8.24 (s, 1H), 8.00 (s, 1H), 7.42 (s, 1H), 5.36 (d, J=4.5 Hz, 1H), 5.11-4.83 (m, 1H), 4.79-4.71 (m, 1H), 3.61 (s, 3H), 2.33-2.15 (m, 4H), 1.79-1.64 (m, 1H), 1.40 (d, J=6.6 Hz, 3H), 1.29-1.15 (m, 1H).

### Example S26. Synthesis of (1S,2S)—N-(3-(6-(2-((tert-butyldimethylsilyl)oxy)propan-2-yl)-4-methylpyridin-3-yl)-1-methyl-2-oxo-1,2-dihydro-1,6-naphthyridin-7-yl)-2-fluorocyclopropane-1-carboxamide (Compound 95)

### Step 1: Synthesis of (1S,2S)—N-(3-(6-(2-((tert-butyldimethylsilyl)oxy)propan-2-yl)-4-methylpyridin-3-yl)-1-methyl-2-oxo-1,2-dihydro-1,6-naphthyridin-7-yl)-2-fluorocyclopropane-1-carboxamide (E-95)

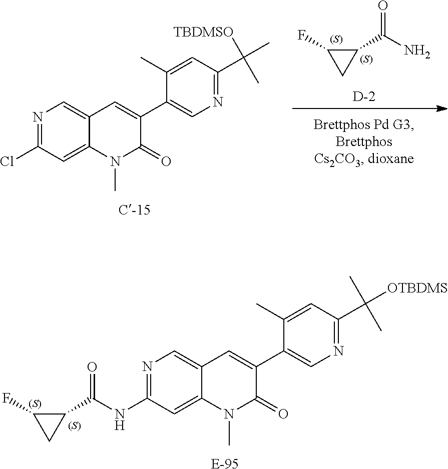

To a solution of 3-(6-(2-((tert-butyldimethylsilyl)oxy)propan-2-yl)-4-methylpyridin-3-yl)-7-chloro-1-methyl-1,6-naphthyridin-2(1H)-one (Compound C′-15) (230.0 mg, 0.50 mmol) in dioxane (10.0 mL) was added (1S,2S)-2-fluorocyclopropane-1-carboxamide (Compound D-2) (310.6 mg, 3.01 mmol), Cs₂CO₃ (490.8 mg, 1.51 mmol), Brettphos (53.9 mg, 0.20 mmol) and BrettPhos Pd G3 (45.5 mg, 0.10 mmol) at room temperature under N₂. The resulting mixture was stirred with microwave radiation at 120° C. for 1.5 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford (1S,2S)—N-(3-(6-(2-((tert-butyldimethylsilyl)oxy)propan-2-yl)-4-methylpyridin-3-yl)-1-methyl-2-oxo-1,2-dihydro-1,6-naphthyridin-7-yl)-2-fluorocyclopropane-1-carboxamide (Compound E-95) (100.0 mg, 37%) as a white solid. LCMS (ESI, m/z): [M+H]⁺=525.3.

### Step 2: Synthesis of (1S,2S)-2-fluoro-N-(3-(6-(2-hydroxypropan-2-yl)-4-methylpyridin-3-yl)-1-methyl-2-oxo-1,2-dihydro-1,6-naphthyridin-7-yl)cyclopropane-1-carboxamide (Compound 95)

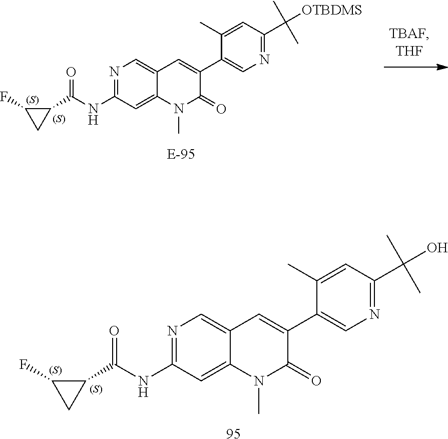

To a solution of (1S,2S)—N-(3-(6-(2-((tert-butyldimethylsilyl)oxy)propan-2-yl)-4-methylpyridin-3-yl)-1-methyl-2-oxo-1,2-dihydro-1,6-naphthyridin-7-yl)-2-fluorocyclopropane-1-carboxamide (Compound E-95) (90.0 mg, 0.17 mmol) in THF (5.0 mL) was added TBAF (89.7 mg, 0.34 mmol) at room temperature. The resulting mixture was stirred at 70° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by reverse phase flash column chromatography with 5-100% CH₃CN in H₂O and then purified by Prep-HPLC with the following conditions (Column: Xselect CSH OBD Column 30×150 mm 5 um; Mobile Phase A: Water (0.1% FA), Mobile Phase B: ACN; Flow rate: 60 mL/min; Gradient: 21% B to 45% B in 7 min; 254 nm) to afford (1S,2S)-2-fluoro-N-(3-(6-(2-hydroxypropan-2-yl)-4-methylpyridin-3-yl)-1-methyl-2-oxo-1,2-dihydro-1,6-naphthyridin-7-yl)cyclopropane-1-carboxamide (Compound 95) (5.8 mg, 8%) as a white solid. LCMS (ESI, m/z): [M+H]⁺=411.2. ¹H NMR (300 MHz, DMSO-d₆): δ 11.32 (s, 1H), 8.71 (s, 1H), 8.27 (s, 1H), 8.20 (s, 1H), 8.02 (s, 1H), 7.58 (s, 1H), 5.24 (s, 1H), 5.09-4.82 (m, 1H), 3.59 (s, 3H), 2.73-2.50 (m, 1H), 2.20 (s, 3H) 1.62-1.58 (m, 1H), 1.47 (s, 6H), 1.35-1.28 (m, 1H).

### Example S27. Synthesis of (R)—N-(3-(6-(1-hydroxypropyl)-4-methylpyridin-3-yl)-1-methyl-2-oxo-1,2-dihydro-1,6-naphthyridin-7-yl)cyclopropanecarboxamide (Compound 68)

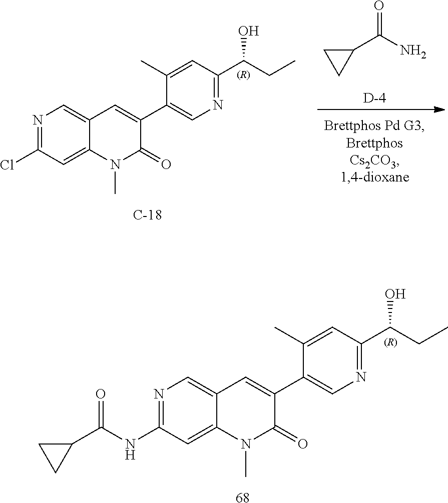

A mixture of 7-chloro-3-[6-[(1R)-1-hydroxypropyl]-4-methylpyridin-3-yl]-1-methyl-1,6-naphthyridin-2-one (Compound C-18) (370.0 mg, 1.07 mmol), cyclopropanecarboxamide (Compound D-4) (109.9 mg, 1.29 mmol), Cs₂CO₃ (1051.9 mg, 3.22 mmol), BrettPhos (231.0 mg, 0.43 mmol) and Brettphos Pd G3 (195.1 mg, 0.21 mmol) in dioxane (6.0 mL) was stirred at 100° C. for 2 h under N₂. After the reaction was completed, the resulting mixture was concentrated under vacuum. The residue was purified by flash column chromatography with CH₂Cl₂/MeOH (10/1, v/v) and then purified by Prep-HPLC with the following conditions (Column: Xselect CSH OBD Column 30×150 mm, 5 um; Mobile Phase A: Water (0.1% FA), Mobile Phase B: ACN; Flow rate: 60 mL/min; Gradient: 9% B to 25% B in 8 min; 254/220 nm) to afford the racemic compound. The racemic compound was purified by CHIRAL-HPLC with the following conditions (Column: CHIRALPAK IA, 2×25 cm, 5 um; Mobile Phase A:Hex:DCM=3:1 (0.5% 2M NH₃-MeOH)-HPLC, Mobile Phase B: EtOH-HPLC; Flow rate:18 mL/min; Gradient:10% B to 10% B in 22 min; 220/254 nm) to afford N-(3-[6-[(1R)-1-hydroxypropyl]-4-methylpyridin-3-yl]-1-methyl-2-oxo-1,6-naphthyridin-7-yl)cyclopropanecarboxamide (Compound 68) (46.5 mg, 3%) as a white solid. LCMS (ESI, m/z): [M+H]⁺=393.2. H NMR (300 MHz, DMSO-d₆): δ 11.16 (s, 1H), 8.71 (s, 1H), 8.27 (s, 1H), 8.25 (s, 1H), 8.01 (s, 1H), 7.40 (s, 1H), 5.31 (d, J=4.2 Hz, 1H), 4.53-4.50 (m, 1H), 3.59 (s, 3H), 2.21 (s, 3H), 2.11-2.00 (m, 1H), 1.90-1.72 (m, 1H), 1.68-1.59 (m, 1H), 0.92-0.86 (m, 7H).

### Example S28. Synthesis of (1S,2S)-2-fluoro-N-[3-(5-fluoro-4-methylpyridin-3-yl)-2-oxo-1H-1,6-naphthyridin-7-yl]cyclopropane-1-carboxamide and (1R,2S)-2-fluoro-N-(3-(5-fluoro-4-methylpyridin-3-yl)-2-oxo-1,2-dihydro-1,6-naphthyridin-7-yl)cyclopropane-1-carboxamide (Compound 38 and 39)

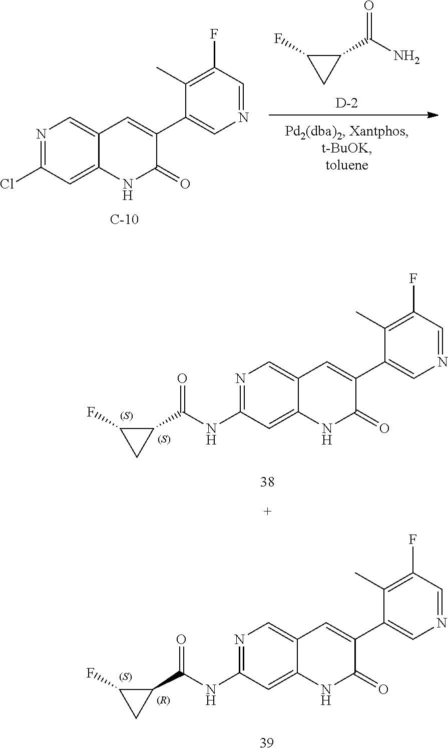

To a solution of 7-chloro-3-(5-fluoro-4-methylpyridin-3-yl)-1H-1,6-naphthyridin-2-one (Compound C-10) (90.0 mg, 0.31 mmol) in 1,4-dioxane (3.0 mL) was added (1S,2S)-2-fluorocyclopropane-1-carboxamide (Compound D-2) (96.1 mg, 0.93 mmol), t-BuOK (104.6 mg, 0.93 mmol), XantPhos (36.0 mg, 0.06 mmol) and Pd₂(dba)₃ (28.2 mg, 0.03 mmol). The resulting mixture was stirred at 100° C. for 3 h under N₂. After the reaction was completed, the resulting mixture was concentrated under vacuum. The residue was purified by flash column chromatography with ether/ethyl acetate (10/90, v/v) and then purified by Prep-HPLC (Column: Xselect CSH F-Phenyl OBD column, 19×250 mm, 5 um; Mobile Phase A: Water (10 mmol/L NH₄HCO₃), Mobile Phase B: MeOH; Flow rate: 25 mL/min; Gradient: 45% B to 60% B in 10 min; 254 nm; retention time 1: 7.9 min; retention time 2: 8.3 min) to afford Isomer A (12.6 mg, 11%) and Isomer B (9.3 mg, 8%) as a white solid.

Isomer A: retention time 1: 7.9 min, LCMS (ESI, m/z): [M+H]⁺=357.2. ¹H NMR (400 MHz, DMSO-d₆): δ 12.27 (s, 1H), 11.07 (s, 1H), 8.69 (s, 1H), 8.53 (s, 1H), 8.31 s, 1H), 8.12 (s, 1H), 8.06 (s, 1H), 5.08-4.84 (m, 1H), 2.28-2.24 (s, 1H), 2.16 (d, J=1.5 Hz, 3H), 1.72-1.64 (m, 1H), 1.24-1.17 (m, 1H).

Isomer B: retention time 2: 8.3 min, LCMS (ESI, m/z): [M+H]⁺=357.1. ¹H NMR (400 MHz, DMSO-d₆): δ 12.29 (s, 1H), 11.17 (s, 1H), 8.69 (s, 1H), 8.52 (s, 1H), 8.31 (s, 1H), 8.07 (s, 1H), 8.06 (s, 1H), 5.05-4.81 (m, 1H), 2.66-2.56 (m, 1H), 2.16 (d, J=1.8 Hz, 3H), 1.63-1.55 (m, 1H), 1.38-1.21 (m, 1H).

The absolute stereochemistry of Isomers A and B was not assigned. The two structures that could be obtained from the mixture as described above are shown as Compounds 38 and 39 in Table 1.

### Example S29. Synthesis of (1S,2S)—N-[1-(cyclopropylmethyl)-3-(5-fluoro-4-methylpyridin-3-yl)-2-oxo-1,6-naphthyridin-7-yl]-2-fluorocyclopropane-1-carboxamide (Compound 58)

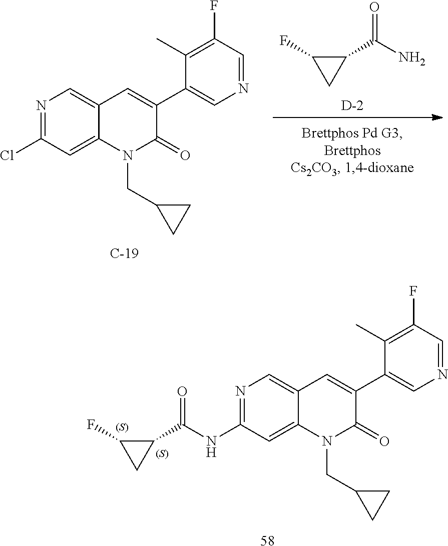

To a solution of 7-chloro-1-(cyclopropylmethyl)-3-(5-fluoro-4-methylpyridin-3-yl)-1,6-naphthyridin-2-one (Compound C-19) (100.0 mg, 0.29 mmol) in 1,4-dioxane (5.0 mL) was added (1S,2S)-2-fluorocyclopropane-1-carboxamide (Compound D-2) (60.0 mg, 0.58 mmol), BrettPhos (31.2 mg, 0.06 mmol), Cs₂CO₃ (284.3 mg, 0.87 mmol) and BrettPhos Pd G3 (26.4 mg, 0.03 mmol). The resulting mixture was irradiated with microwave radiation at 110° C. for 1 h under N₂. After the reaction was completed, the resulting mixture was concentrated under vacuum. The residue was purified by flash column chromatography with CH₂Cl₂/MeOH (95/5, v/v) and then purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 28% B to 53% B in 10 min; 254 nm) to afford (1S,2S)—N-[1-(cyclopropylmethyl)-3-(5-fluoro-4-methylpyridin-3-yl)-2-oxo-1,6-naphthyridin-7-yl]-2-fluorocyclopropane-1-carboxamide (Compound 58) (8.2 mg, 7%) as a white solid. LCMS (ESI, m/z): [M+H]⁺=411.2. ¹H NMR (300 MHz, DMSO-d₆): δ 11.24 (s, 1H), 8.76 (s, 1H), 8.53 (s, 1H), 8.40 (s, 1H), 8.33 (s, 1H), 8.11 (s, 1H), 5.07-4.89 (m, 1H), 4.17-4.11 (m, 2H), 2.32-2.27 (m, 1H), 2.13 (s, 3H), 1.74-1.68 (m, 1H), 1.27-1.24 (m, 2H), 0.6-0.4 (m, 4H).

### Example S30. Synthesis of (1S,2S)-2-fluoro-N-(3-(6-((S)-1-hydroxyethyl)-4-methylpyridin-3-yl)-1-methyl-2-oxo-1,2-dihydro-1,6-naphthyridin-7-yl)cyclopropane-1-carboxamide (Compound 87)

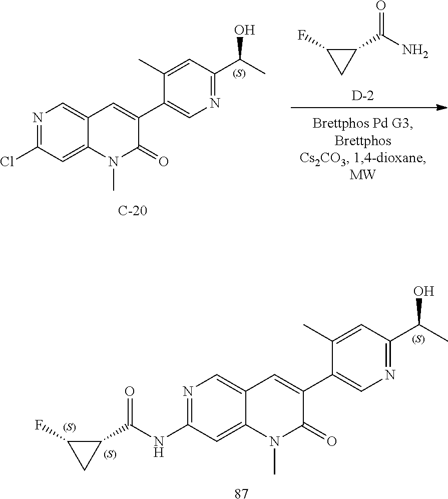

A mixture of 7-chloro-3-[6-[(1S)-1-hydroxyethyl]-4-methylpyridin-3-yl]-1-methyl-1,6-naphthyridin-2-one (Compound C-20) (240.0 mg, 0.72 mmol), (1S,2S)-2-fluorocyclopropane-1-carboxamide (Compound D-2) (375.1 mg, 3.63 mmol), BrettPhos (156.2 mg, 0.29 mmol), Cs₂CO₃ (711.3 mg, 2.18 mmol) and BrettPhos Pd G3 (131.9 mg, 0.14 mmol) in dioxane (10.0 mL) was irradiated with microwave radiation at 120° C. for 1 h under N₂. After the reaction was completed, the resulting mixture was diluted with H₂O and extracted with ethyl acetate. The combined organic layer was washed with brine, dried over anhydrous Na₂SO₄ and filtered. The filtrate was concentrated under vacuum. The residue was purified by flash column chromatography with CH₂Cl₂/CH₃OH (9/1, v/v) and then purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 17% B to 47% B in 7 min; 254 nm) to afford (1S,2S)-2-fluoro-N-(3-[6-[(1S)-1-hydroxyethyl]-4-methylpyridin-3-yl]-1-methyl-2-oxo-1,6-naphthyridin-7-yl)cyclopropane-1-carboxamide (Compound 87) (6.9 mg, 2%) as a white solid. LCMS (ESI, m/z): [M+H]⁺=397.1. ¹H NMR (400 MHz, DMSO-d₆): δ 11.18 (s, 1H), 8.71 (s, 1H), 8.26 (s, 1H), 8.25 (s, 1H), 8.00 (s, 1H), 7.43 (s, 1H), 5.35 (d, J=4.8 Hz, 1H), 5.08-5.85 (m, 1H), 4.75-4.70 (m, 1H), 3.61 (s, 3H), 2.36-2.23 (m, 1H), 2.20 (s, 3H), 1.75-1.63 (m, 1H), 1.40 (d, J=6.4 Hz, 3H), 1.30-1.18 (m, 1H).

### Example S31. Synthesis of (1R,2S)-2-fluoro-N-(3-(5-fluoro-4-methylpyridin-3-yl)-1-methyl-2-oxo-1,2-dihydro-1,6-naphthyridin-7-yl)cyclopropane-1-carboxamide (Compound 29)

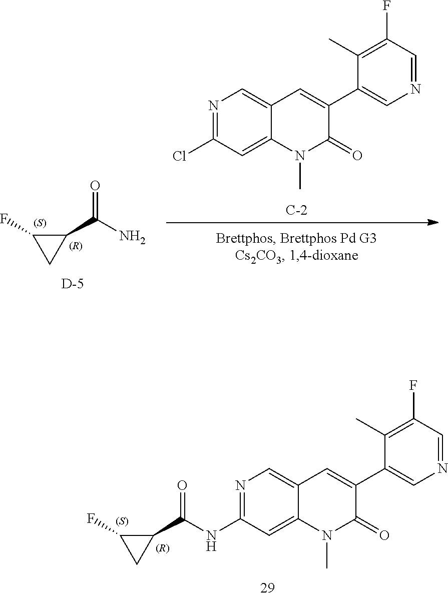

To a solution of 7-chloro-3-(5-fluoro-4-methylpyridin-3-yl)-1-methyl-1,6-naphthyridin-2(1H)-one (Compound C-2) (120.0 mg, 0.40 mmol) in 1,4-dioxane (6.0 mL) was added (1R,2S)-2-fluorocyclopropane-1-carboxamide (Compound D-5) (203.7 mg, 1.98 mmol), Cs₂CO₃ (386.2 mg, 1.19 mmol), BrettPhos (42.4 mg, 0.08 mmol) and BrettPhos Pd G3 (35.8 mg, 0.04 mmol) at room temperature under N₂. The resulting mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (1/99, v/v) to afford (1R,2S)-2-fluoro-N-(3-(5-fluoro-4-methylpyridin-3-yl)-1-methyl-2-oxo-1,2-dihydro-1,6-naphthyridin-7-yl)cyclopropane-1-carboxamide (Compound 29) (7.1 mg, 5%) as a white solid. LCMS (ESI, m/z): [M+H]⁺=371.2. ¹H NMR (300 MHz, DMSO-d₆): δ 11.34 (s, 1H), 8.75 (s, 1H), 8.53 (s, 1H), 8.30 (s, 1H), 8.20 (s, 1H), 8.09 (s, 1H), 5.06-4.85 (m, 1H), 3.59 (s, 3H), 2.73-2.58 (m, 1H), 2.14 (s, 3H), 1.63-1.53 (m, 1H), 1.35-1.25 (m, 1H).

### Example S32. Synthesis of (1S,2R)-2-fluoro-N-[3-(5-fluoro-4-methylpyridin-3-yl)-1-methyl-2-oxo-1,6-naphthyridin-7-yl]cyclopropane-1-carboxamide (Compound 30)

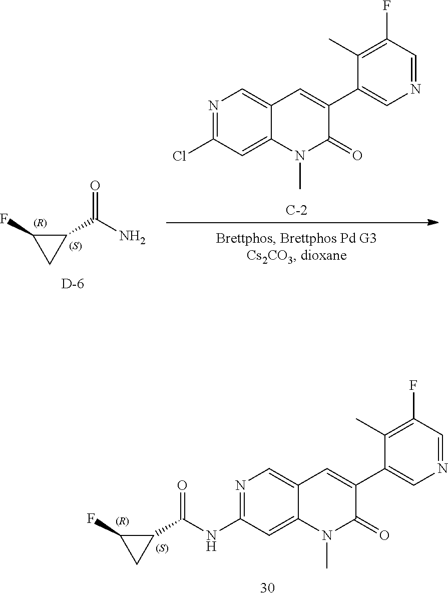

To a solution of 7-chloro-3-(5-fluoro-4-methylpyridin-3-yl)-1-methyl-1,6-naphthyridin-2-one (Compound C-2) (130.0 mg, 0.43 mmol) in 1,4-dioxane (5.0 mL) was added (1S,2R)-2-fluorocyclopropane-1-carboxamide (Compound D-6) (220.6 mg, crude), BrettPhos (45.9 mg, 0.07 mmol), Cs₂CO₃ (418.4 mg, 1.28 mmol) and BrettPhos Pd G3 (38.8 mg, 0.04 mmol) at room temperature under N₂. The resulting mixture was stirred at 100° C. for 4 h. After the reaction was completed, the reaction mixture was diluted with water and extracted with ethyl acetate. The combined organic layer was washed with brine, dried over anhydrous sodium sulfate and filtered. The filtrate was concentrated under reduced pressure. The residue was purified by Prep-HPLC with the following conditions Column: (Xcelect CSH F-pheny OBD Column, 19×250 mm, 5 um; Mobile Phase A: Water (10 mmol/L NH₄HCO₃), Mobile Phase B: ACN; Flow rate: 25 mL/min; Gradient: 50% B to 69% B in 7 min; 254 nm) to afford (1S,2R)-2-fluoro-N-[3-(5-fluoro-4-methylpyridin-3-yl)-1-methyl-2-oxo-1,6-naphthyridin-7-yl]cyclopropane-1-carboxamide (Compound 30) (25.3 mg, 16%) as a white solid. LCMS (ESI, m/z): [M+H]⁺=371.2. ¹H NMR (400 MHz, DMSO-d₆): δ 11.32 (s, 1H), 8.74 (s, 1H), 8.53 (s, 1H), 8.29 (s, 1H), 8.20 (s, 1H), 8.08 (s, 1H), 5.03-4.86 (m, 1H), 3.59 (s, 3H), 2.68-2.59 (m, 1H), 2.13 (s, 3H), 1.63-1.22 (m, 1H), 1.41-1.22 (m, 1H).

### Example S33. Synthesis of (1S,2S)—N-[3-(5-cyano-4-methylpyridin-3-yl)-1-methyl-2-oxo-1,6-naphthyridin-7-yl]-2-fluorocyclopropane-1-carboxamide (Compound 99)

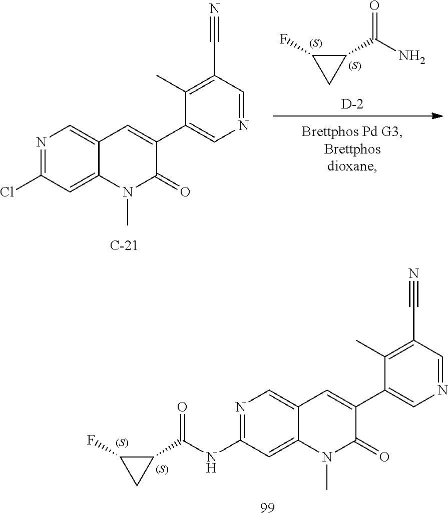

To a solution of 5-(7-chloro-1-methyl-2-oxo-1,6-naphthyridin-3-yl)-4-methylpyridine-3-carbonitrile (Compound C-21) (100.0 mg, 0.32 mmol) in dioxane (5.0 mL) was added (1S,2S)-2-fluorocyclopropane-1-carboxamide (Compound D-2) (165.8 mg, 1.60 mmol), Brettphos Pd G3 (58.3 mg, 0.06 mmol), BrettPhos (69.1 mg, 0.12 mmol) and K₂CO₃ (133.4 mg, 0.96 mmol) at room temperature under N₂. The final reaction mixture was irradiated with microwave radiation at 100° C. for 1 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (90/10, v/v) and then purified by Prep-HPLC with the following conditions (Column: YMC-Actus Triart C18, 30×250 mm, 5 um; Mobile Phase A: Water (10 mmol/L NH₄HCO₃), Mobile Phase B: ACN; Flow rate: 60 mL/min; Gradient: 29% B to 38% B in 8 min; 254/220 nm) to afford (1S,2S)—N-[3-(5-cyano-4-methylpyridin-3-yl)-1-methyl-2-oxo-1,6-naphthyridin-7-yl]-2-fluorocyclopropane-1-carboxamide (Compound 99) (12.8 mg, 10%) as a white solid. LCMS (ESI, m/z): [M+H]⁺=378.2. ¹H NMR (400 MHz, DMSO-d₆): δ 11.25 (s, 1H), 8.99 (s, 1H), 8.74 (s, 1H), 8.68 (s, 1H), 8.27 (s, 1H), 8.12 (s, 1H), 5.07-4.89 (m, 1H), 3.62 (s, 3H), 2.38 (s, 3H), 2.33-2.28 (m, 1H), 1.73-1.67 (m, 1H), 1.26-1.24 (m, 1H).

### Example S34. Synthesis of (1S,2S)-2-fluoro-N-(3-[6-[(1S)-1-hydroxypropyl]-4-methylpyridin-3-yl]-2-oxo-1H-1,6-naphthyridin-7-yl)cyclopropane-1-carboxamide and (1S,2S)-2-fluoro-N-(3-[6-[(1R)-1-hydroxypropyl]-4-methylpyridin-3-yl]-2-oxo-1H-1,6-naphthyridin-7-yl)cyclopropane-1-carboxamide (Compound 100 and 101)

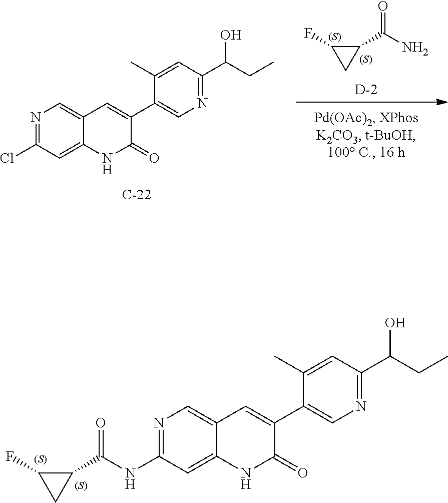

To a solution of 7-chloro-3-[6-(1-hydroxypropyl)-4-methylpyridin-3-yl]-1H-1,6-naphthyridin-2-one (Compound C-22) (450.0 mg, 1.37 mmol) in t-BuOH (30.0 mL) was added (1S,2S)-2-fluorocyclopropane-1-carboxamide (Compound D-2) (422.0 mg, 4.09 mmol), K₂CO₃ (209.5 mg, 1.52 mmol), XPhos (130.1 mg, 0.27 mmol) and Pd(OAc)₂ (17.0 mg, 0.08 mmol) at room temperature under N₂. The resulting mixture was stirred at 100° C. for 16 h. After the reaction was completed, the resulting mixture was filtered. The filtrate was concentrated under reduced pressure. The residue was purified by flash chromatography with CH₂Cl₂/MeOH (10/1, v/v) to afford (1S,2S)-2-fluoro-N-[3-[6-(1-hydroxypropyl)-4-methylpyridin-3-yl]-2-oxo-1H-1,6-naphthyridin-7-yl]cyclopropane-1-carboxamide (Compound 100 and 101) (79.0 mg, 26%) as a white solid. LCMS (ESI, m/z): [M+H]⁺=397.2.

### Example S35. Chiral Separation of (1S,2S)-2-fluoro-N-(3-[6-[(1S)-1-hydroxypropyl]-4-methylpyridin-3-yl]-2-oxo-1H-1,6-naphthyridin-7-yl)cyclopropane-1-carboxamide and (1S,2S)-2-fluoro-N-(3-[6-[(1R)-1-hydroxypropyl]-4-methylpyridin-3-yl]-2-oxo-1H-1,6-naphthyridin-7-yl)cyclopropane-1-carboxamide (Compound 100 and 101)

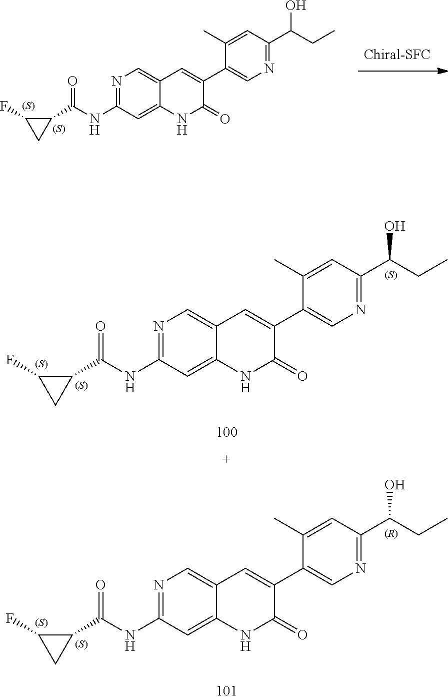

The racemic of (1S,2S)-2-fluoro-N-[3-[6-(1-hydroxypropyl)-4-methylpyridin-3-yl]-2-oxo-1H-1,6-naphthyridin-7-yl]cyclopropane-1-carboxamide (79.0 mg, 0.20 mmol) was separated by Prep-Chiral-HPLC with the following conditions (Column: CHIRALPAK IA, 2×25 cm, 5 um; Mobile Phase A:Hex:DCM=3:1 (0.5% 2M NH₃-MeOH)-HPLC, Mobile Phase B: EtOH-HPLC; Flow rate:20 mL/min; Gradient:50% B to 50% B in 13 min; 254/220 nm; retention time 1: 6.588 min; retention time 2: 10.132 min) to afford Enantiomer A (7.0 mg, 8%) as a white solid and Enantiomer B (9.9 mg, 12%) as a white solid.

Enantiomer A: retention time 1: 6.588 min, LCMS (ESI, m/z): [M+H]⁺=397.0. ¹H NMR (300 MHz, DMSO-d₆): δ 12.20 (s, 1H), 11.06 (s, 1H), 8.67 (s, 1H), 8.28 (s, 1H), 8.11 (s, 1H), 7.98 (s, 1H), 7.39 (s, 1H), 5.33 (d, J=3.6 Hz, 1H), 5.10-4.82 (m, 1H), 4.55-4.48 (m, 1H), 2.28-2.23 (m, 4H), 1.82-1.63 (m, 3H), 1.26-1.15 (m, 1H), 0.92-0.87 (m, 3H).

Enantiomer B: retention time 2: 10.132 min, LCMS (ESI, m/z): [M+H]⁺=397.0. ¹H NMR (300 MHz, DMSO-d₆): δ 12.20 (s, 1H), 11.06 (s, 1H), 8.67 (s, 1H), 8.28 (s, 1H), 8.11 (s, 1H), 7.99 (s, 1H), 7.40 (s, 1H), 5.34 (s, 1H), 5.10-4.83 (m, 1H), 4.55-4.48 (m, 1H), 2.28-2.23 (m, 4H), 1.82-1.63 (m, 3H), 1.26-1.15 (m, 1H), 0.92-0.87 (m, 3H).

The absolute stereochemistry of enantiomers A and B was not assigned. The two enantiomeric structures that could be obtained from chiral separation of the enantiomeric mixture as described above are shown as Compounds 100 and 101 in Table 1.

### Example S36. Synthesis of (1S,2S)-2-fluoro-N-[3-(5-fluoro-2-methylphenyl)-1-methyl-2-oxo-1,6-naphthyridin-7-yl]cyclopropane-1-carboxamide (Compound 102)

### Step 1. Synthesis of 7-chloro-3-(5-fluoro-2-methylphenyl)-1-methyl-1,6-naphthyridin-2-one

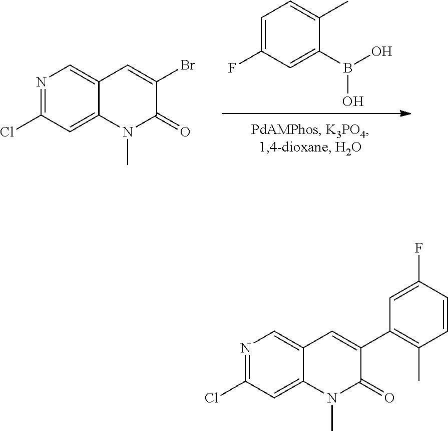 [SMILES: Cn1c(=O)c(Br)cc2cnc(Cl)cc21 ; Cc1ccc(F)cc1B(O)O ; Cc1ccc(F)cc1-c1cc2cnc(Cl)cc2n(C)c1=O]

To a solution of 3-bromo-7-chloro-1-methyl-1,6-naphthyridin-2-one (2.0 g, 7.31 mmol) in 1,4-dioxane/H₂O (20.0/4.0 mL) was added 5-fluoro-2-methylphenylboronic acid (1.1 g, 7.31 mmol), K₃PO₄ (4.7 g, 21.94 mmol) and PdAMPhos (517.8 mg, 0.73 mmol) at room temperature under N₂. The resulting mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (90/10, v/v) to afford 7-chloro-3-(5-fluoro-2-methylphenyl)-1-methyl-1,6-naphthyridin-2-one (800.0 mg, 36%) as a white solid. LCMS (ESI, m/z): [M+H]⁺=303.1.

### Step 2. Synthesis of (1S,2S)-2-fluoro-N-[3-(5-fluoro-2-methylphenyl)-1-methyl-2-oxo-1,6-naphthyridin-7-yl]cyclopropane-1-carboxamide (Compound 102)

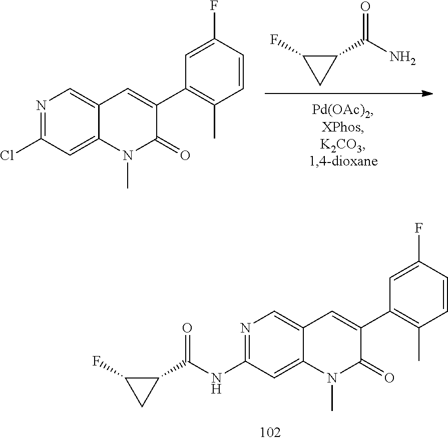

To a solution of 7-chloro-3-(5-fluoro-2-methylphenyl)-1-methyl-1,6-naphthyridin-2-one (100.0 mg, 0.33 mmol) in 1,4-dioxane (1.0 mL) was added (1S,2S)-2-fluorocyclopropane-1-carboxamide (170.3 mg, 1.65 mmol), K₂CO₃ (137.0 mg, 0.99 mmol), Pd(OAc)₂ (7.4 mg, 0.03 mmol) and XPhos (31.4 mg, 0.07 mmol) at room temperature under N₂. The resulting mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/CH₃OH (90/10, v/v) and then purified by Prep-HPLC with the following conditions (Column: XBridge Prep OBD C18 Column, 30×150 mm, 5 m; Mobile Phase A: Water (10 mmol/L NH₄HCO₃), Mobile Phase B: ACN; Flow rate: 60 mL/min; Gradient: 35% B to 45% B in 8 min; Wave Length: 254 nm) to afford (1S,2S)-2-fluoro-N-[3-(5-fluoro-2-methylphenyl)-1-methyl-2-oxo-1,6-naphthyridin-7-yl]cyclopropane-1-carboxamide (Compound 102) (36.2 mg, 29%) as a white solid. LCMS (ESI, m/z): [M+H]⁺=370.1. ¹H NMR (400 MHz, DMSO-d₆): δ 11.18 (s, 1H), 8.71 (s, 1H), 8.24 (s, 1H), 7.95 (s, 1H), 7.33-7.30 (m, 1H), 7.16-7.07 (m, 2H), 5.06-4.87 (m, 1H), 3.61 (s, 3H), 2.33-2.25 (m, 1H), 2.14 (s, 3H), 1.73-1.68 (m, 1H), 1.30-1.20 (m, 1H).

### Example S37. Synthesis of N-(3-(5-fluoro-2-methylphenyl)-1-methyl-2-oxo-1,2-dihydro-1,6-naphthyridin-7-yl)cyclopropanecarboxamide (Compound 103)

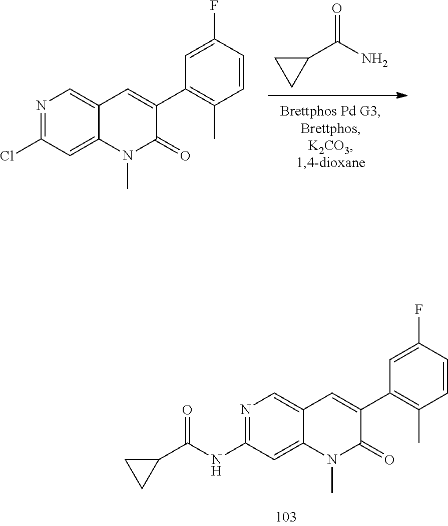

To a solution of 7-chloro-3-(5-fluoro-2-methylphenyl)-1-methyl-1,6-naphthyridin-2(1H)-one (300.0 mg, 0.99 mmol) in 1,4-dioxane (10.0 mL) was added cyclopropanecarboxamide (421.7 mg, 4.96 mmol), K₂CO₃ (410.9 mg, 2.98 mmol), Brettphos (106.4 mg, 0.20 mmol) and Brettphos Pd G3 (89.8 mg, 0.10 mmol) at room temperature under N₂. The resulting mixture was stirred at 100° C. for 2 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/CH₃OH (90/10, v/v) and then purified by Prep-HPLC with the following conditions (Column: XBridge Prep OBD C18 Column, 30×150 mm, 5 m; Mobile Phase A: Water (10 mmol/L NH₄HCO₃), Mobile Phase B: ACN; Flow rate: 60 mL/min; Gradient: 38% B to 48% B in 8 min; Wave Length: 254 nm) to afford N-(3-(5-fluoro-2-methylphenyl)-1-methyl-2-oxo-1,2-dihydro-1,6-naphthyridin-7-yl)cyclopropanecarboxamide (Compound 103) (33.1 mg, 9%) as a white solid. LCMS (ESI, m/z): [M+H]⁺=352.1. ¹H NMR (400 MHz, DMSO-d₆): δ 11.14 (s, 1H), 8.69 (s, 1H), 8.24 (s, 1H), 7.94 (s, 1H), 7.33-7.29 (m, 1H), 7.16-7.06 (m, 2H), 3.59 (s, 3H), 2.13-1.97 (m, 4H), 0.89-0.84 (m, 4H).

### Example S38. Synthesis of (1S,2S)—N-(3-(2-chloro-6-methylphenyl)-1-methyl-2-oxo-1,2-dihydro-1,6-naphthyridin-7-yl)-2-fluorocyclopropane-1-carboxamide (Compound 104)

### Step 1. Synthesis of 2-(2-chloro-6-methylphenyl)-4,4,5,5-tetramethyl-1,3,2-dioxaborolane

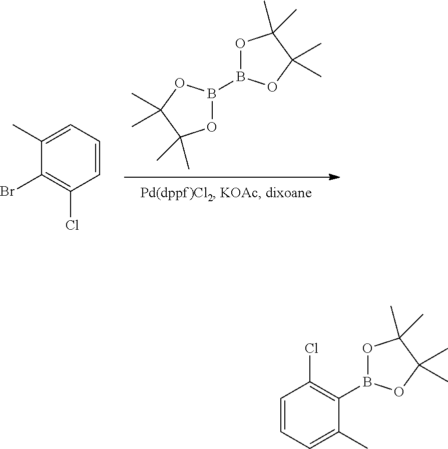

To a solution of 2-bromo-1-chloro-3-methylbenzene (500.0 mg, 2.23 mmol) in dioxane (20.0 mL) was added bis(pinacolato)diboron (1.7 g, 6.68 mmol), KOAc (655.9 mg, 6.68 mmol) and Pd(dppf)Cl₂ (363.5 mg, 0.45 mmol) at room temperature under N₂. The resulting mixture was stirred at 100° C. for 4 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/2, v/v) to afford 2-(2-chloro-6-methylphenyl)-4,4,5,5-tetramethyl-1,3,2-dioxaborolane (576.0 mg, 19%) as a white oil. LCMS (ESI, m/z): [M+H]⁺=253.1.

### Step 2. Synthesis of 7-chloro-3-(2-chloro-6-methylphenyl)-1-methyl-1,6-naphthyridin-2-one

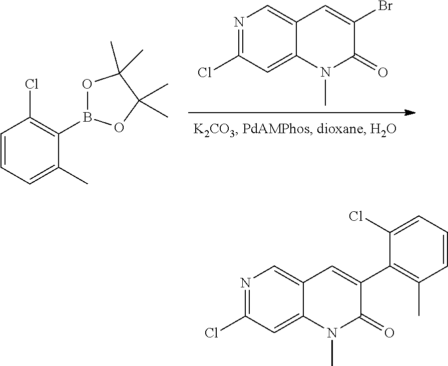

To a solution of 3-bromo-7-chloro-1-methyl-1,6-naphthyridin-2-one (1.6 g, 5.85 mmol) in dioxane/H₂O (15.0 mL/3.0 mL) was added 2-(2-chloro-6-methylphenyl)-4,4,5,5-tetramethyl-1,3,2-dioxaborolane (1.8 g, 7.02 mmol), K₂CO₃ (2.4 g, 17.55 mmol) and PdAMPhos (0.4 g, 0.59 mmol) at room temperature under N₂. The resulting mixture was stirred at 80° C. for 3 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford 7-chloro-3-(2-chloro-6-methylphenyl)-1-methyl-1,6-naphthyridin-2-one (1.9 g, 89%) as an off-white solid. LCMS (ESI, m/z): [M+H]⁺=319.0.

### Step 3. Synthesis of (1S,2S)—N-(3-(2-chloro-6-methylphenyl)-1-methyl-2-oxo-1,2-dihydro-1,6-naphthyridin-7-yl)-2-fluorocyclopropane-1-carboxamide (Compound 104)

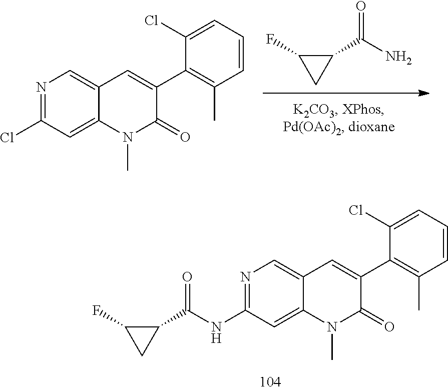

To a solution of 7-chloro-3-(2-chloro-6-methylphenyl)-1-methyl-1,6-naphthyridin-2-one (500.0 mg, 1.57 mmol) in dioxane (20.0 mL) was added (1S,2S)-2-fluorocyclopropane-1-carboxamide (807.5 mg, 7.83 mmol), K₂CO₃ (649.5 mg, 4.70 mmol), XPhos (149.4 mg, 0.31 mmol) and Pd(OAc)₂ (35.2 mg, 0.16 mmol) at room temperature under N₂. The resulting mixture was stirred at 100° C. for 4 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/2, v/v) and then purified by Prep-HPLC with the following conditions (Column: Aeris PEPTIDE 5 um XB-C18 Axia, 21.2 mm×250 mm, 5 m; Mobile Phase A: Water (10 mmol/L NH₄HCO₃), Mobile Phase B: ACN; Flow rate: 60 mL/min; Gradient: 30% B to 45% B in 12 min, Wave Length: 254 nm) to afford (1S,2S)—N-(3-(2-chloro-6-methylphenyl)-1-methyl-2-oxo-1,2-dihydro-1,6-naphthyridin-7-yl)-2-fluorocyclopropane-1-carboxamide (Compound 104) (51.7 mg, 8%) as a white solid. LCMS (ESI, m/z): [M+H]⁺=368.1. ¹H NMR (400 MHz, DMSO-d₆): δ 11.21 (s, 1H), 8.71 (s, 1H), 8.26 (d, J=1.6 Hz, 1H), 7.92 (s, 1H), 7.39-7.28 (m, 3H), 5.08-4.87 (m, 1H), 3.62 (s, 3H), 2.35-2.29 (m, 1H), 2.12 (s, 3H), 1.75-1.68 (m, 1H), 1.27-1.17 (m, 1H).

### Example S39. Synthesis of N-(3-(2-chloro-6-methylphenyl)-1-methyl-2-oxo-1,2-dihydro-1,6-naphthyridin-7-yl)cyclopropanecarboxamide (Compound 105)

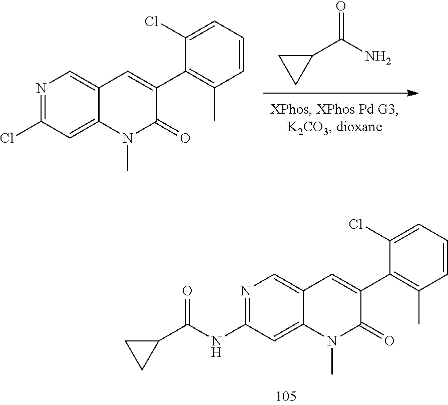

To a solution of 7-chloro-3-(2-chloro-6-methylphenyl)-1-methyl-1,6-naphthyridin-2-one (500.0 mg, 1.57 mmol) in 1,4-dioxane (20.0 mL) was added cyclopropanecarboxamide (666.6 mg, 7.83 mmol), K₂CO₃ (649.5 mg, 4.70 mmol), XPhos (149.4 mg, 0.31 mmol) and XPhos Pd G3 (132.6 mg, 0.16 mmol) at room temperature under N₂. The resulting mixture was stirred at 100° C. for 4 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) and then purified by Prep-HPLC with the following conditions (Column: XBridge Shield RP18 OBD Column, 30×150 mm, 5 μm; Mobile Phase A: Water (10 mmol/L NH₄HCO₃), Mobile Phase B: ACN; Flow rate: 60 mL/min; Gradient: 30% B to 45% B in 12 min, Wave Length: 254 nm) to afford N-[3-(2-chloro-6-methylphenyl)-1-methyl-2-oxo-1,6-naphthyridin-7-yl]cyclopropanecarboxamide (Compound 105) (47.4 mg, 8%) as a white solid. LCMS (ESI, m/z): [M+H]⁺=368.1. ¹H NMR (400 MHz, DMSO-d₆): δ 11.15 (s, 1H), 8.69 (d, J=5.6 Hz, 1H), 8.26 (s, 1H), 7.90 (s, 1H), 7.39-7.27 (m, 3H), 3.60 (s, 3H), 2.16-2.07 (m, 4H), 0.92-0.86 (m, 4H).

### Example S40. Synthesis of (1S,2S)-2-fluoro-N-(3-(6-((R)-1-hydroxypropyl)-4-methylpyridin-3-yl)-1-methyl-2-oxo-1,2-dihydro-1,6-naphthyridin-7-yl) cyclopropane-1-carboxamide and (1S,2S)-2-fluoro-N-(3-(6-((S)-1-hydroxypropyl)-4-methylpyridin-3-yl)-1-methyl-2-oxo-1,2-dihydro-1,6-naphthyridin-7-yl) cyclopropane-1-carboxamide (Compound 106 and 107)

### Step 1. Synthesis of (1S,2S)-2-fluoro-N-(1-methyl-3-(4-methyl-6-propionylpyridin-3-yl)-2-oxo-1,2-dihydro-1,6-naphthyridin-7-yl) cyclopropane-1-carboxamide

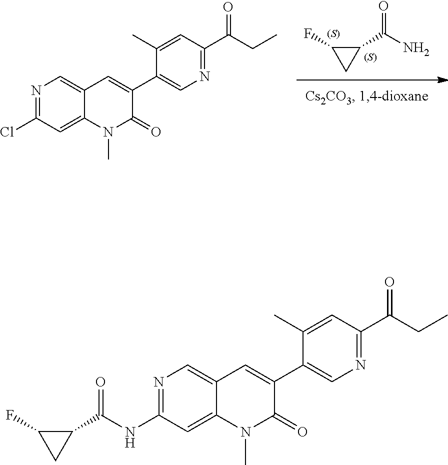

To a mixture of 7-chloro-1-methyl-3-(4-methyl-6-propanoylpyridin-3-yl)-1,6-naphthyridin-2-one (600.0 mg, 1.75 mmol) and (1S,2S)-2-fluorocyclopropane-1-carboxamide (361.9 mg, 3.51 mmol) in dioxane (10.0 mL) was added Cs₂CO₃ (1143.9 mg, 3.51 mmol), BrettPhos Pd G3 (159.1 mg, 0.17 mmol) and BrettPhos (188.4 mg, 0.35 mmol) at room temperature under N₂. The resulting mixture was stirred at 80° C. for 4 h. After the reaction was completed, the resulting mixture was diluted with water and extracted with ethyl acetate. The combined organic layers were washed with brine, dried over anhydrous Na₂SO₄ and filtered. The filtrate was concentrated under reduced pressure. The residue was purified by flash column chromatography with ethyl acetate/petroleum ether (99/1, v/v) to afford (1S,2S)-2-fluoro-N-(1-methyl-3-(4-methyl-6-propionylpyridin-3-yl)-2-oxo-1,2-dihydro-1,6-naphthyridin-7-yl) cyclopropane-1-carboxamide (400.0 mg, 55%) as a white solid. LCMS (ESI, m/z): [M+H]⁺=409.1.

### Step 2. Synthesis of (1S,2S)-2-fluoro-N-(3-(6-(1-hydroxypropyl)-4-methylpyridin-3-yl)-1-methyl-2-oxo-1,2-dihydro-1,6-naphthyridin-7-yl) cyclopropane-1-carboxamide

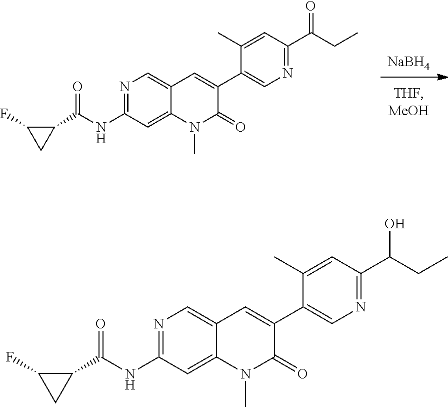 [SMILES: CCC(=O)c1cc(C)c(-c2cc3cnc(NC(=O)[C@@H]4C[C@@H]4F)cc3n(C)c2=O)cn1 ; "NaBH4" ; CCC(O)c1cc(C)c(-c2cc3cnc(NC(=O)[C@@H]4C[C@@H]4F)cc3n(C)c2=O)cn1]

To a solution of (1S,2S)-2-fluoro-N-(1-methyl-3-(4-methyl-6-propionylpyridin-3-yl)-2-oxo-1,2-dihydro-1,6-naphthyridin-7-yl) cyclopropane-1-carboxamide (400.0 mg, 0.97 mmol) in THF (10.0 mL) and MeOH (1.0 mL) was added NaBH₄ (185.2 mg, 4.89 mmol) at 0° C. The resulting mixture was stirred at room temperature for 2 h. After the reaction was completed, the reaction mixture was diluted with H₂O and extracted with ethyl acetate. The combined organic layers were washed with brine, dried over anhydrous Na₂SO₄ and filtered. The filtrate was concentrated under reduced pressure. The residue was purified by flash column chromatography with ethyl acetate/methanol (9/1, v/v) to afford (1S,2S)-2-fluoro-N-(3-(6-(1-hydroxypropyl)-4-methylpyridin-3-yl)-1-methyl-2-oxo-1,2-dihydro-1,6-naphthyridin-7-yl) cyclopropane-1-carboxamide (300.0 mg, 74%) as a white solid. LCMS (ESI, m/z): [M+H]⁺=411.1.

### Step 3. Chiral Separation of (1S,2S)-2-fluoro-N-(3-(6-((R)-1-hydroxypropyl)-4-methylpyridin-3-yl)-1-methyl-2-oxo-1,2-dihydro-1,6-naphthyridin-7-yl) cyclopropane-1-carboxamide and (1S,2S)-2-fluoro-N-(3-(6-((S)-1-hydroxypropyl)-4-methylpyridin-3-yl)-1-methyl-2-oxo-1,2-dihydro-1,6-naphthyridin-7-yl) cyclopropane-1-carboxamide (Compound 106 and 107)

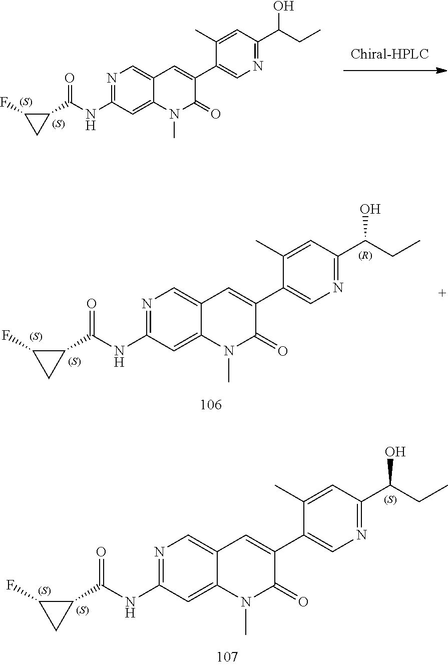

The product (1S,2S)-2-fluoro-N-(3-(6-(1-hydroxypropyl)-4-methylpyridin-3-yl)-1-methyl-2-oxo-1,2-dihydro-1,6-naphthyridin-7-yl) cyclopropane-1-carboxamide (160.0 mg, 0.39 mmol) was separated by Pre-Chiral-HPLC with the following conditions (Column: CHIRAL ART Amylose-SA, 2×25 cm, 5 m; Mobile Phase A: Hex (0.5% 2M NH₃-MeOH)-HPLC, Mobile Phase B: EtOH:DCM=1:1-HPLC; Flow rate: 20 mL/min; Gradient: 35% B to 35% B in 19 min; Wave Length: 220/254 nm; RT1(min): 14.10; RT2(min): 17.28) to afford (1S,2S)-2-fluoro-N-(3-(6-(1-hydroxypropyl)-4-methylpyridin-3-yl)-1-methyl-2-oxo-1,2-dihydro-1,6-naphthyridin-7-yl) cyclopropane-1-carboxamide Isomer A (retention time: 14.10 min, 50.2 mg, 62%) as a white solid and (1S,2S)-2-fluoro-N-(3-(6-(1-hydroxypropyl)-4-methylpyridin-3-yl)-1-methyl-2-oxo-1,2-dihydro-1,6-naphthyridin-7-yl) cyclopropane-1-carboxamide Isomer B (retention time: 17.28 min, 50.0 mg, 62%) as a white solid.

Isomer A: retention time 1: 14.10 min; LCMS (ESI, m/z): [M+H]⁺=411.1. ¹H NMR (400 MHz, DMSO-d₆): δ 11.20 (s, 1H), 8.72 (s, 1H), 8.27-8.25 (m, 2H), 8.01 (s, 1H), 7.40 (s, 1H), 5.31 (d, J=4.8 Hz, 1H), 5.07-4.89 (m, 1H), 4.54-4.45 (m, 1H), 3.61 (s, 3H), 2.36-2.26 (m, 1H), 2.21 (s, 3H), 1.85-1.79 (m, 1H), 1.77-1.62 (m, 2H), 1.35-1.18 (m, 1H), 0.94-0.87 (m, 3H).

Isomer B: retention time 2: 17.28 min; LCMS (ESI, m/z): [M+H]⁺=411.1. ¹H NMR (400 MHz, DMSO-d₆): δ 11.20 (s, 1H), 8.71 (s, 1H), 8.27-8.25 (m, 2H), 8.01 (s, 1H), 7.40 (s, 1H), 5.31 (d, J=4.8 Hz, 1H), 5.08-4.88 (m, 1H), 4.52-4.47 (m, 1H), 3.61 (s, 3H), 2.36-2.26 (m, 1H), 2.21 (s, 3H), 1.86-1.60 (m, 3H), 1.27-1.19 (m, 1H), 0.92-0.88 (m, 3H).

The absolute stereochemistry of Isomer A and B was not assigned. The two isomeric structures that could be obtained from chiral separation of the isomeric mixture as described above are shown as Compounds 106 and 107 in Table 1.

### Example S41. Synthesis of (1R,2R)-2-fluoro-N-(3-(6-((S)-1-hydroxypropyl)-4-methylpyridin-3-yl)-1-methyl-2-oxo-1,2-dihydro-1,6-naphthyridin-7-yl)cyclopropane-1-carboxamide and (1R,2R)-2-fluoro-N-(3-(6-((R)-1-hydroxypropyl)-4-methylpyridin-3-yl)-1-methyl-2-oxo-1,2-dihydro-1,6-naphthyridin-7-yl)cyclopropane-1-carboxamide (Compound 108 and 109)

### Step 1. Synthesis of (1R,2R)-2-fluoro-N-(1-methyl-3-(4-methyl-6-propionylpyridin-3-yl)-2-oxo-1,2-dihydro-1,6-naphthyridin-7-yl)cyclopropane-1-carboxamide

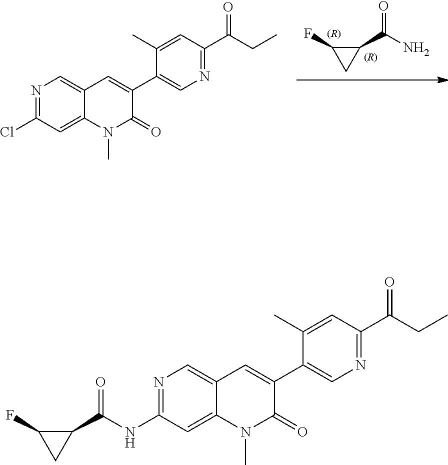

To a stirred mixture of 7-chloro-1-methyl-3-(4-methyl-6-propanoylpyridin-3-yl)-1,6-naphthyridin-2-one (800.0 mg, 2.34 mmol), (1R,2R)-2-fluorocyclopropane-1-carboxamide (482.6 mg, 4.68 mmol) and Cs₂CO₃ (1.5 g, 4.68 mmol) in 1,4-dioxane (20.0 mL) were added Pd(OAc)₂ (52.5 mg, 0.23 mmol) and XPhos (223.1 mg, 0.46 mmol) at room temperature. The resulting mixture was stirred at 80° C. for 3 h under N₂. After the reaction was completed, the mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford (1R,2R)-2-fluoro-N-(1-methyl-3-(4-methyl-6-propionylpyridin-3-yl)-2-oxo-1,2-dihydro-1,6-naphthyridin-7-yl)cyclopropane-1-carboxamide (544.0 mg, 54%) as a yellow solid. LCMS (ESI, m/z): [M+H]⁺=409.2.

### Step 2. Synthesis of (1R,2R)-2-fluoro-N-(3-(6-(1-hydroxypropyl)-4-methylpyridin-3-yl)-1-methyl-2-oxo-1,2-dihydro-1,6-naphthyridin-7-yl)cyclopropane-1-carboxamide

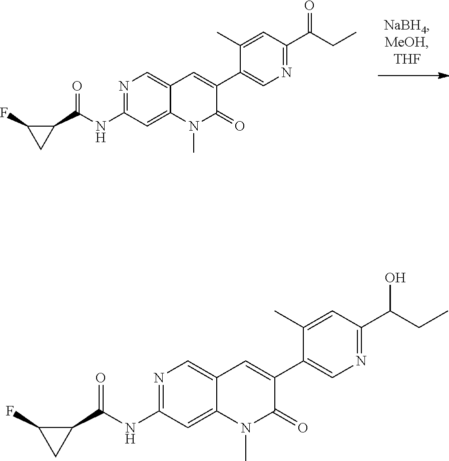 [SMILES: CCC(=O)c1cc(C)c(-c2cc3cnc(NC(=O)[C@H]4C[C@H]4F)cc3n(C)c2=O)cn1 ; CCC(O)c1cc(C)c(-c2cc3cnc(NC(=O)[C@H]4C[C@H]4F)cc3n(C)c2=O)cn1]

To a stirred mixture of (1R,2R)-2-fluoro-N-(1-methyl-3-(4-methyl-6-propionylpyridin-3-yl)-2-oxo-1,2-dihydro-1,6-naphthyridin-7-yl)cyclopropane-1-carboxamide (490.0 mg, 1.20 mmol) in MeOH (1.5 mL) and THE (15.0 mL) was added NaBH₄ (226.9 mg, 6.00 mmol) at 0° C. The resulting mixture was stirred at room temperature for 4 h under N₂. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/4, v/v) to afford (1R,2R)-2-fluoro-N-(3-(6-(1-hydroxypropyl)-4-methylpyridin-3-yl)-1-methyl-2-oxo-1,2-dihydro-1,6-naphthyridin-7-yl)cyclopropane-1-carboxamide (515.3 mg, 95%) as a yellow solid. LCMS (ESI, m/z): [M+H]⁺=411.2.

### Step 3. Chiral Separation (1R,2R)-2-fluoro-N-(3-(6-((S)-1-hydroxypropyl)-4-methylpyridin-3-yl)-1-methyl-2-oxo-1,2-dihydro-1,6-naphthyridin-7-yl)cyclopropane-1-carboxamide and (1R,2R)-2-fluoro-N-(3-(6-((R)-1-hydroxypropyl)-4-methylpyridin-3-yl)-1-methyl-2-oxo-1,2-dihydro-1,6-naphthyridin-7-yl)cyclopropane-1-carboxamide (Compound 108 and 109)

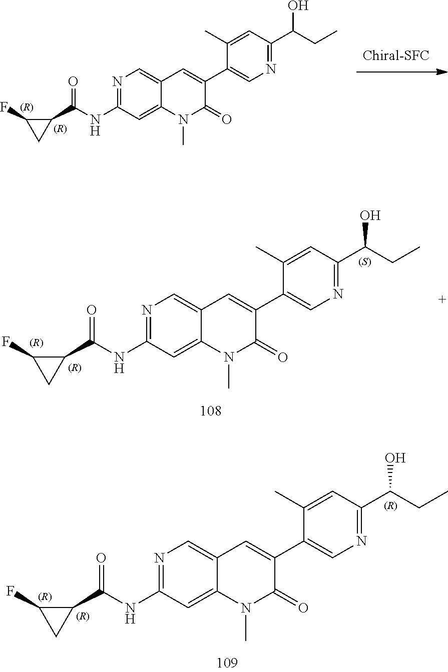

The product (1R,2R)-2-fluoro-N-(3-(6-(1-hydroxypropyl)-4-methylpyridin-3-yl)-1-methyl-2-oxo-1,2-dihydro-1,6-naphthyridin-7-yl)cyclopropane-1-carboxamide (200.0 mg, 0.49 mmol) was separated by Prep-Chiral-HPLC with the following conditions (Column: CHIRAL ART Amylose-SA, 2×25 cm, 5 m; Mobile Phase A: MtBE (0.5% 2M NH₃-MeOH)-HPLC, Mobile Phase B: EtOH:DCM=1:1-HPLC; Flow rate: 20 mL/min; Gradient: 20% B to 20% B in 18.5 min; Wave Length: 220/254 nm; RT1(min): 11.45; RT2(min): 15.90) to afford (1R,2R)-2-fluoro-N-(3-(6-((S)-1-hydroxypropyl)-4-methylpyridin-3-yl)-1-methyl-2-oxo-1,2-dihydro-1,6-naphthyridin-7-yl)cyclopropane-1-carboxamide (retention time 11.45 min, 47.7 mg, 47%) as a white solid and (1R,2R)-2-fluoro-N-(3-(6-((R)-1-hydroxypropyl)-4-methylpyridin-3-yl)-1-methyl-2-oxo-1,2-dihydro-1,6-naphthyridin-7-yl)cyclopropane-1-carboxamide (retention time 15.90 min, 52.2 mg, 52%) as a white solid.

(1R,2R)-2-fluoro-N-(3-(6-((S)-1-hydroxypropyl)-4-methylpyridin-3-yl)-1-methyl-2-oxo-1,2-dihydro-1,6-naphthyridin-7-yl)cyclopropane-1-carboxamide: retention time 1: 11.45 min; LCMS (ESI, m/z): [M+H]⁺=411.2. ¹H NMR (400 MHz, DMSO-d₆): δ 11.18 (s, 1H), 8.71 (s, 1H), 8.26 (d, J=8.4 Hz, 2H), 8.01 (s, 1H), 7.39 (s, 1H), 5.29 (d, J=4.8 Hz, 1H), 5.07-4.89 (m, 1H), 4.53-4.49 (m, 1H), 3.62 (s, 3H), 2.31-2.27 (m, 1H), 2.20 (s, 3H), 1.84-1.79 (m, 1H), 1.75-1.62 (m, 2H), 1.27-1.20 (m, 1H), 0.92-0.85 (m, 3H). The absolute stereochemistry of the isomer was resolved by x-ray crystallography.

(1R,2R)-2-fluoro-N-(3-(6-((R)-1-hydroxypropyl)-4-methylpyridin-3-yl)-1-methyl-2-oxo-1,2-dihydro-1,6-naphthyridin-7-yl)cyclopropane-1-carboxamide: retention time 2: 15.9 min; LCMS (ESI, m/z): [M+H]⁺=411.3. ¹H NMR (400 MHz, DMSO-d₆): δ 11.18 (s, 1H), 8.71 (s, 1H), 8.26 (d, J=8.8 Hz, 2H), 8.01 (s, 1H), 7.40 (s, 1H), 5.30 (d, J=3.6 Hz, 1H), 5.08-4.87 (m, 1H), 4.52-4.47 (m, 1H), 3.61 (s, 3H), 2.33-2.26 (m, 1H), 2.21 (s, 3H), 1.86-1.79 (m, 1H), 1.75-1.62 (m, 2H), 1.27-1.18 (m, 1H), 0.92-0.88 (m, 3H).

### Example S42. Synthesis of (R)—N-(3-(6-(1-hydroxybutyl)-4-methylpyridin-3-yl)-1-methyl-2-oxo-1,2-dihydro-1,6-naphthyridin-7-yl)cyclopropanecarboxamide and (S)—N-(3-(6-(1-hydroxybutyl)-4-methylpyridin-3-yl)-1-methyl-2-oxo-1,2-dihydro-1,6-naphthyridin-7-yl)cyclopropanecarboxamide (Compound 110 and 111)

### Step 1. Synthesis of 3-(6-butyryl-4-methylpyridin-3-yl)-7-chloro-1-methyl-1,6-naphthyridin-2(1H)-one

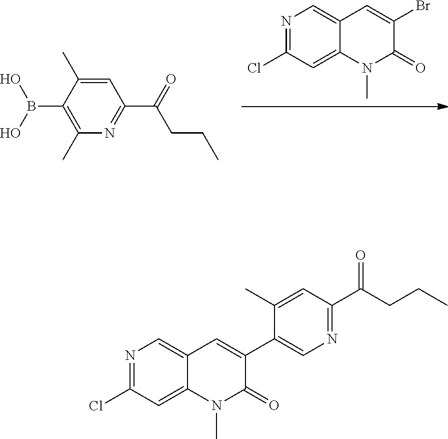 [SMILES: CCCC(=O)c1cc(C)c(B(O)O)c(C)n1 ; Cn1c(=O)c(Br)cc2cnc(Cl)cc21 ; CCCC(=O)c1cc(C)c(-c2cc3cnc(Cl)cc3n(C)c2=O)cn1]

To a stirred mixture of 6-butanoyl-4-methylpyridin-3-ylboronic acid (1.5 g, 6.52 mmol), 3-bromo-7-chloro-1-methyl-1,6-naphthyridin-2-one (1.7 g, 6.52 mmol) and Pd(dppf)Cl₂ (480.0 mg, 0.65 mmol) in 1,4-dioxane (200.0 mL) and H₂O (40.0 mL) was added K₂CO₃ (2.7 g, 19.56 mmol) at room temperature. The resulting mixture was stirred at 85° C. for 4 h. After the reaction was completed, the mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (60/40, v/v) to afford 3-(6-butanoyl-4-methylpyridin-3-yl)-7-chloro-1-methyl-1,6-naphthyridin-2-one (1.3 g, 44%) as a pink solid. LCMS (ESI, m/z): [M+H]⁺=356.1.

### Step 2. Synthesis of N-(3-(6-butyryl-4-methylpyridin-3-yl)-1-methyl-2-oxo-1,2-dihydro-1,6-naphthyridin-7-yl)cyclopropanecarboxamide

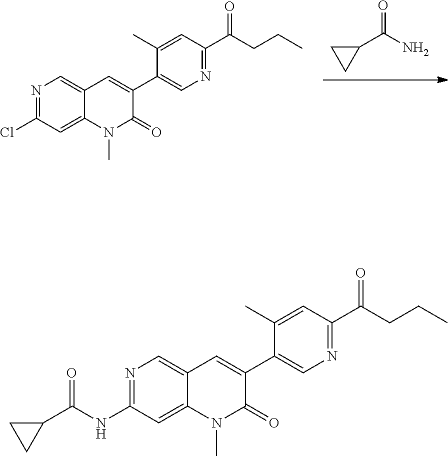

To a stirred mixture of 3-(6-butanoyl-4-methylpyridin-3-yl)-7-chloro-1-methyl-1,6-naphthyridin-2-one (1.2 g, 2.63 mmol), cyclopropanecarboxamide (450.0 mg, 5.26 mmol) and Cs₂CO₃ (1.7 g, 5.26 mmol) in 1,4-dioxane (100.0 mL) were added Pd(OAc)₂ (60.0 mg, 0.26 mmol) and XPhos (250.0 mg, 0.52 mmol) at room temperature. The resulting mixture was stirred at 100° C. for 4 h under N₂. After the reaction was completed, the mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford N-[3-(6-butanoyl-4-methylpyridin-3-yl)-1-methyl-2-oxo-1,6-naphthyridin-7-yl]cyclopropanecarboxamide (1.1 g, 99%) as a white solid. LCMS (ESI, m/z): [M+H]⁺=405.2.

### Step 3. Synthesis of N-(3-(6-(1-hydroxybutyl)-4-methylpyridin-3-yl)-1-methyl-2-oxo-1,2-dihydro-1,6-naphthyridin-7-yl)cyclopropanecarboxamide

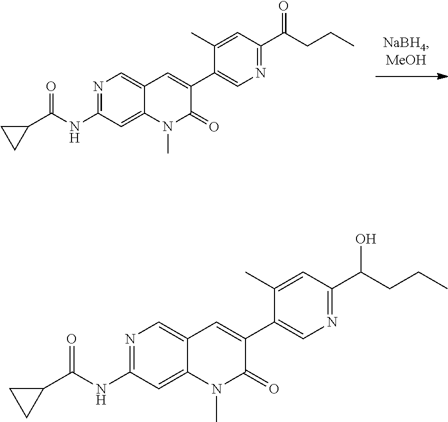 [SMILES: CCCC(=O)c1cc(C)c(-c2cc3cnc(NC(=O)C4CC4)cc3n(C)c2=O)cn1 ; CCCC(O)c1cc(C)c(-c2cc3cnc(NC(=O)C4CC4)cc3n(C)c2=O)cn1]

To a stirred mixture of N-[3-(6-butanoyl-4-methylpyridin-3-yl)-1-methyl-2-oxo-1,6-naphthyridin-7-yl]cyclopropanecarboxamide (500.0 mg, 1.23 mmol) in MeOH (2.0 mL) and THE (20.0 mL) was added NaBH₄ (233.8 mg, 6.18 mmol) at 0° C. The resulting mixture was stirred at room temperature for 4 h. After the reaction was completed, the reaction mixture was quenched with NH₄Cl (aq.) at 0° C. The resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/4, v/v) to afford N-{3-[6-(1-hydroxybutyl)-4-methylpyridin-3-yl]-1-methyl-2-oxo-1,6-naphthyridin-7-yl}cyclopropanecarboxamide (260.0 mg, 46%) as a white solid. LCMS (ESI, m/z): [M+H]⁺=407.2.

### Step 4. Chiral Separation of (R)—N-(3-(6-(1-hydroxybutyl)-4-methylpyridin-3-yl)-1-methyl-2-oxo-1,2-dihydro-1,6-naphthyridin-7-yl)cyclopropanecarboxamide and (S)—N-(3-(6-(1-hydroxybutyl)-4-methylpyridin-3-yl)-1-methyl-2-oxo-1,2-dihydro-1,6-naphthyridin-7-yl)cyclopropanecarboxamide (Compound 110 and 111)

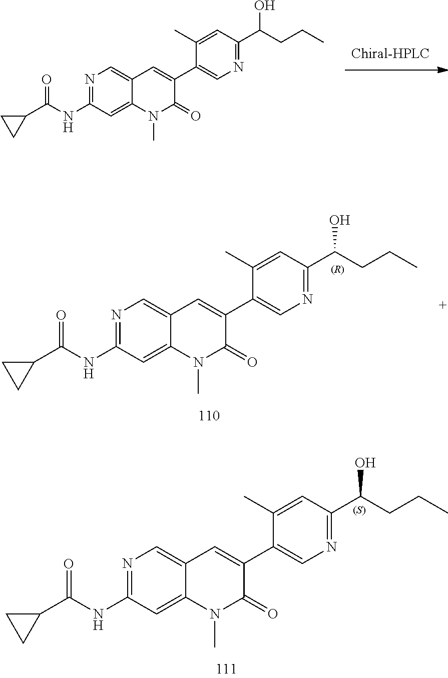

The product of N-{3-[6-(1-hydroxybutyl)-4-methylpyridin-3-yl]-1-methyl-2-oxo-1,6-naphthyridin-7-yl}cyclopropanecarboxamide (236.0 mg, 0.58 mmol) was separated by Prep-Chiral-HPLC with the following conditions (Column: CHIRAL ART Cellulose-SC, 2×25 cm, 5 m; Mobile Phase A: Hex (0.5% 2M NH₃-MeOH)-HPLC, Mobile Phase B: EtOH:DCM=1:1-HPLC; Flow rate: 20 mL/min; Gradient: 55% B to 55% B in 14 min; Wave Length: 254 nm; RT1(min): 10.20; RT2(min): 12.01) to afford N-(3-(6-(1-hydroxybutyl)-4-methylpyridin-3-yl)-1-methyl-2-oxo-1,2-dihydro-1,6-naphthyridin-7-yl)cyclopropanecarboxamide Enantiomer A (retention time 10.20 min 72.1 mg, 61%) as a white solid and N-(3-(6-(1-hydroxybutyl)-4-methylpyridin-3-yl)-1-methyl-2-oxo-1,2-dihydro-1,6-naphthyridin-7-yl)cyclopropanecarboxamide Enantiomer B (retention time 12.01 min, 41.5 mg, 35%) as a white solid.

Enantiomer A: retention time 1: 10.20 min, LCMS (ESI, m/z): [M+H]⁺=407.3. ¹H NMR (400 MHz, DMSO-d₆): δ 11.13 (s, 1H), 8.70 (s, 1H), 8.26-8.25 (m, 2H), 7.99 (s, 1H), 7.39 (s, 1H), 5.27 (d, J=4.8 Hz, 1H), 4.60-4.55 (m, 1H), 3.59 (s, 3H), 2.20 (s, 3H), 2.11-2.04 (m, 1H), 1.79-1.58 (m, 2H), 1.45-1.30 (m, 2H), 0.98-0.82 (m, 7H).

Enantiomer B: retention time 2: 12.01 min, LCMS (ESI, m/z): [M+H]⁺=407.3. ¹H NMR (400 MHz, DMSO-d₆): δ 11.13 (s, 1H), 8.70 (s, 1H), 8.25 (s, 2H), 7.99 (s, 1H), 7.39 (s, 1H), 5.27 (d, J=5.2 Hz, 1H), 4.60-4.55 (m, 1H), 3.59 (s, 3H), 2.20 (s, 3H), 2.09-2.04 (m, 1H), 1.78-1.69 (m, 1H), 1.66-1.57 (m, 1H), 1.41-1.35 (m, 2H), 0.92-0.86 (m, 7H).

The absolute stereochemistry of enantiomers A and B was not assigned. The two enantiomeric structures that could be obtained from chiral separation of the enantiomeric mixture as described above are shown as Compounds 110 and 111 in Table 1.

### Example S43. Synthesis of (1R,2R)-2-fluoro-N-(3-(6-((S)-1-hydroxybutyl)-4-methylpyridin-3-yl)-1-methyl-2-oxo-1,2-dihydro-1,6-naphthyridin-7-yl)cyclopropane-1-carboxamide and (1R,2R)-2-fluoro-N-(3-(6-((R)-1-hydroxybutyl)-4-methylpyridin-3-yl)-1-methyl-2-oxo-1,2-dihydro-1,6-naphthyridin-7-yl)cyclopropane-1-carboxamide (Compound 112 and 113)

### Step 1. Synthesis of 3-(6-butyryl-4-methylpyridin-3-yl)-7-chloro-1-methyl-1,6-naphthyridin-2(1H)-one

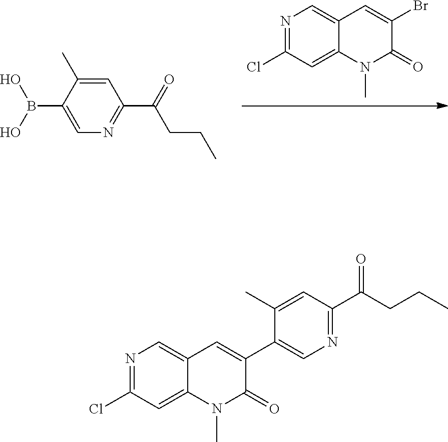 [SMILES: CCCC(=O)c1cc(C)c(B(O)O)cn1 ; Cn1c(=O)c(Br)cc2cnc(Cl)cc21 ; CCCC(=O)c1cc(C)c(-c2cc3cnc(Cl)cc3n(C)c2=O)cn1]

To a stirred mixture of 6-butanoyl-4-methylpyridin-3-ylboronic acid (750.0 mg, 3.26 mmol), 3-bromo-7-chloro-1-methyl-1,6-naphthyridin-2-one (891.7 mg, 3.26 mmol) and Pd(dppf)Cl₂ (238.6 mg, 0.33 mmol) in 1,4-dioxane (15.0 mL) and H₂O (3.0 mL) was added K₂CO₃ (1.3 g, 9.78 mmol) at room temperature. The resulting mixture was stirred at 85° C. for 4 h. After the reaction was completed, the mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (3/2, v/v) to afford 3-(6-butanoyl-4-methylpyridin-3-yl)-7-chloro-1-methyl-1,6-naphthyridin-2-one (760.0 mg, 58%) as a pink solid. LCMS (ESI, m/z): [M+H]⁺=356.1.

### Step 2. Synthesis of (1R,2R)—N-(3-(6-butyryl-4-methylpyridin-3-yl)-1-methyl-2-oxo-1,2-dihydro-1,6-naphthyridin-7-yl)-2-fluorocyclopropane-1-carboxamide

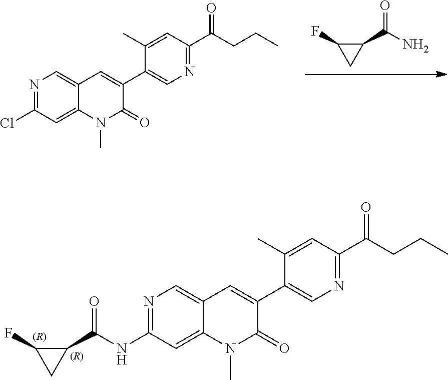

To a stirred mixture of 3-(6-butanoyl-4-methylpyridin-3-yl)-7-chloro-1-methyl-1,6-naphthyridin-2-one (740.0 mg, 2.08 mmol), (1R,2R)-2-fluorocyclopropane-1-carboxamide (857.6 mg, 8.32 mmol) and Cs₂CO₃ (1355.2 mg, 4.16 mmol) in 1,4-dioxane (20.0 mL) were added Pd(OAc)₂ (46.6 mg, 0.20 mmol) and XPhos (198.3 mg, 0.42 mmol) at room temperature. The resulting mixture was stirred at 80° C. for 3 h under N₂. After the reaction was completed, the mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with ether/ethyl acetate (1/1, v/v) to afford (1R,2R)—N-(3-(6-butyryl-4-methylpyridin-3-yl)-1-methyl-2-oxo-1,2-dihydro-1,6-naphthyridin-7-yl)-2-fluorocyclopropane-1-carboxamide (900.0 mg, 95%) as a yellow solid. LCMS (ESI, m/z): [M+H]⁺=423.2.

### Step 3. Synthesis of (1R,2R)-2-fluoro-N-(3-(6-(1-hydroxybutyl)-4-methylpyridin-3-yl)-1-methyl-2-oxo-1,2-dihydro-1,6-naphthyridin-7-yl)cyclopropane-1-carboxamide

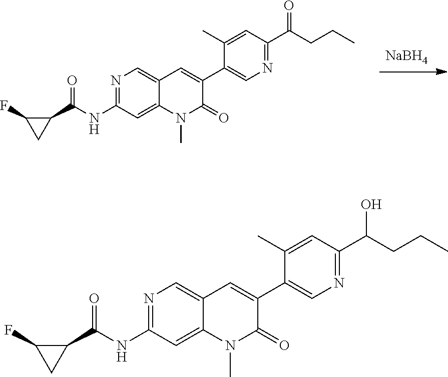 [SMILES: CCCC(=O)c1cc(C)c(-c2cc3cnc(NC(=O)[C@H]4C[C@H]4F)cc3n(C)c2=O)cn1 ; "NaBH4" ; CCCC(O)c1cc(C)c(-c2cc3cnc(NC(=O)[C@H]4C[C@H]4F)cc3n(C)c2=O)cn1]

To a stirred mixture of (1R,2R)—N-[3-(6-butanoyl-4-methylpyridin-3-yl)-1-methyl-2-oxo-1,6-naphthyridin-7-yl]-2-fluorocyclopropane-1-carboxamide (400.0 mg, 0.98 mmol) in MeOH (2.0 mL) and THE (20.0 mL) was added NaBH₄ (179.1 mg, 4.74 mmol) at 0° C. The resulting mixture was stirred at 0° C. for 3 h. After the reaction was completed, the reaction mixture was quenched with NH₄Cl (aq.) at 0° C. The mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/MeOH (9/1, v/v) to afford (1R,2R)-2-fluoro-N-{3-[6-(1-hydroxybutyl)-4-methylpyridin-3-yl]-1-methyl-2-oxo-1,6-naphthyridin-7-yl}cyclopropane-1-carboxamide (60.0 mg, 14%) as a yellow solid. LCMS (ESI, m/z): [M+H]⁺=425.2.

### Step 4. Chiral Separation of (1R,2R)-2-fluoro-N-(3-(6-((S)-1-hydroxybutyl)-4-methylpyridin-3-yl)-1-methyl-2-oxo-1,2-dihydro-1,6-naphthyridin-7-yl)cyclopropane-1-carboxamide and (1R,2R)-2-fluoro-N-(3-(6-((R)-1-hydroxybutyl)-4-methylpyridin-3-yl)-1-methyl-2-oxo-1,2-dihydro-1,6-naphthyridin-7-yl)cyclopropane-1-carboxamide (Compound 112 and 113)

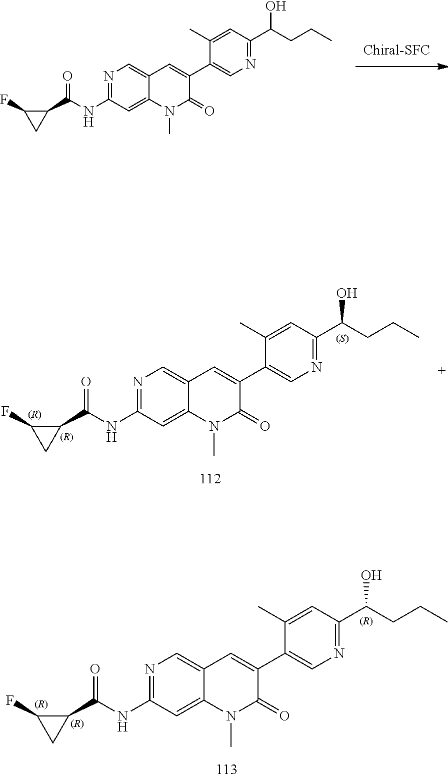

The product of (1R,2R)-2-fluoro-N-{3-[6-(1-hydroxybutyl)-4-methylpyridin-3-yl]-1-methyl-2-oxo-1,6-naphthyridin-7-yl}cyclopropane-1-carboxamide (160.0 mg, 0.38 mmol) was separated by Prep-Chiral-HPLC with the following conditions (Column: CHIRAL ART Amylose-SA, 2×25 cm, 5 m; Mobile Phase A: MtBE (0.1% FA)-HPLC, Mobile Phase B: EtOH:DCM=1:1-HPLC; Flow rate: 20 mL/min; Gradient: 20% B to 20% B in 18 min; Wave Length: 220/254 nm; RT1 (min): 12.08; RT2 (min): 15.71) to afford (1R,2R)-2-fluoro-N-(3-(6-(1-hydroxybutyl)-4-methylpyridin-3-yl)-1-methyl-2-oxo-1,2-dihydro-1,6-naphthyridin-7-yl)cyclopropane-1-carboxamide Isomer A (retention time 12.08 min, 55.6 mg, 69%) as a white solid and (1R,2R)-2-fluoro-N-(3-(6-(1-hydroxybutyl)-4-methylpyridin-3-yl)-1-methyl-2-oxo-1,2-dihydro-1,6-naphthyridin-7-yl)cyclopropane-1-carboxamide Isomer B (retention time 15.71 min, 52.5 mg, 65%) as a white solid.

Isomer A: retention time 1: 12.08 min, LCMS (ESI, m/z): [M+H]⁺=425.3. ¹H NMR (400 MHz, DMSO-d₆): δ 11.21 (s, 1H), 8.72 (s, 1H), 8.26-8.25 (m, 2H), 8.01 (s, 1H), 7.40 (s, 1H), 5.31 (d, J=5.2 Hz, 1H), 5.07-4.89 (m, 1H), 4.59-4.57 (m, 1H), 3.61 (s, 3H), 2.31-2.27 (m, 1H), 2.20 (s, 3H), 1.75-1.61 (m, 3H), 1.42-1.35 (m, 2H), 1.25-1.20 (m, 1H), 0.92-0.88 (m, 3H).

Isomer B: retention time 2: 15.71 min, LCMS (ESI, m/z): [M+H]⁺=425.2. ¹H NMR (400 MHz, DMSO-d₆): δ 11.21 (s, 1H), 8.72 (s, 1H), 8.26-8.25 (m, 2H), 8.01 (s, 1H), 7.40 (s, 1H), 5.31 (d, J=4.8 Hz, 1H), 5.07-4.89 (m, 1H), 4.60-4.56 (m, 1H), 3.61 (s, 3H), 2.30-2.27 (m, 1H), 2.20 (s, 3H), 1.75-1.61 (m, 3H), 1.42-1.35 (m, 2H), 1.25-1.20 (m, 1H), 0.92-0.88 (m, 3H).

The absolute stereochemistry of Isomers A and B was not assigned. The two isomeric structures that could be obtained from chiral separation of the isomeric mixture as described above are shown as Compounds 112 and 113 in Table 1.

### Example S44. Synthesis of (1S,2S)-2-fluoro-N-(3-(6-((S)-1-hydroxybutyl)-4-methylpyridin-3-yl)-1-methyl-2-oxo-1,2-dihydro-1,6-naphthyridin-7-yl)cyclopropane-1-carboxamide and (1S,2S)-2-fluoro-N-(3-(6-((R)-1-hydroxybutyl)-4-methylpyridin-3-yl)-1-methyl-2-oxo-1,2-dihydro-1,6-naphthyridin-7-yl)cyclopropane-1-carboxamide (Compound 114 and 115)

### Step 1. Synthesis of (1S,2S)-2-fluoro-N-(3-(6-(1-hydroxybutyl)-4-methylpyridin-3-yl)-1-methyl-2-oxo-1,2-dihydro-1,6-naphthyridin-7-yl)cyclopropane-1-carboxamide

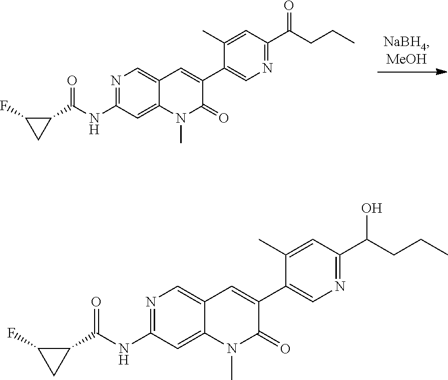

To a solution of (1S,2S)—N-(3-(6-butyryl-4-methylpyridin-3-yl)-1-methyl-2-oxo-1,2-dihydro-1,6-naphthyridin-7-yl)-2-fluorocyclopropane-1-carboxamide (220.0 mg, 0.52 mmol) in THF (6.0 mL) and MeOH (0.6 mL) were added and NaBH₄ (98.5 mg, 2.60 mmol) at 0° C. under N₂. The resulting mixture was stirred at room temperature for 2 h. After the reaction was completed, the reaction mixture was quenched with H₂O at 0° C. The resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/MeOH (10/1, v/v) to afford (1S,2S)-2-fluoro-N-(3-(6-(1-hydroxybutyl)-4-methylpyridin-3-yl)-1-methyl-2-oxo-1,2-dihydro-1,6-naphthyridin-7-yl)cyclopropane-1-carboxamide (140.0 mg, 63%) as a light yellow solid. LCMS (ESI, m/z): [M+H]⁺=425.2.

### Step 2. Chiral Separation of (1S,2S)-2-fluoro-N-(3-(6-((S)-1-hydroxybutyl)-4-methylpyridin-3-yl)-1-methyl-2-oxo-1,2-dihydro-1,6-naphthyridin-7-yl)cyclopropane-1-carboxamide and (1S,2S)-2-fluoro-N-(3-(6-((R)-1-hydroxybutyl)-4-methylpyridin-3-yl)-1-methyl-2-oxo-1,2-dihydro-1,6-naphthyridin-7-yl)cyclopropane-1-carboxamide (Compound 114 and 115)

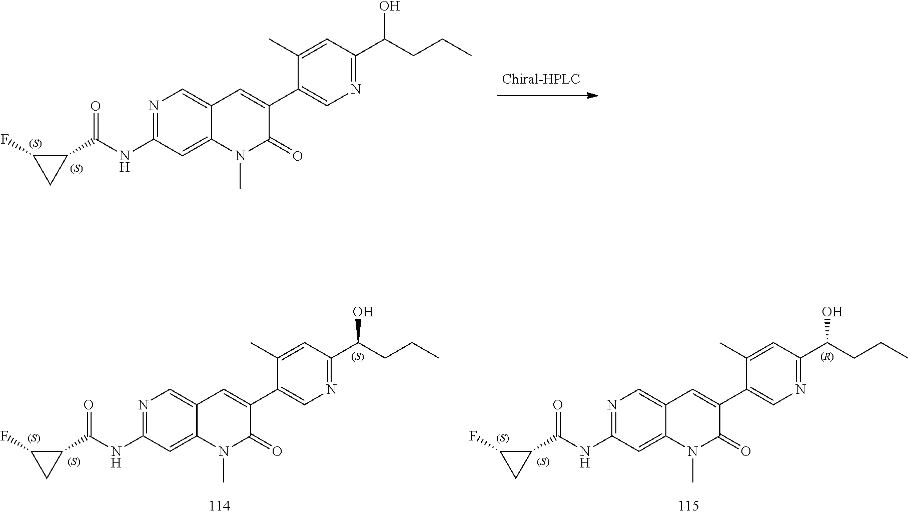

The product of (1S,2S)-2-fluoro-N-(3-(6-(1-hydroxybutyl)-4-methylpyridin-3-yl)-1-methyl-2-oxo-1,2-dihydro-1,6-naphthyridin-7-yl)cyclopropane-1-carboxamide (80.0 mg, 0.18 mmol) was separated by Prep-Chiral-HPLC with the following conditions (Column: CHIRAL ART Cellulose-SB, 2×25 cm, 5 m; Mobile Phase A: Hex (0.5% 2M NH₃-MeOH)-HPLC, Mobile Phase B: MeOH:DCM=1:1-HPLC; Flow rate: 20 mL/min; Gradient: 25% B to 25% B in 18.5 min; Wave Length: 254/220 nm; RT1(min): 12.99; RT2(min): 16.18) to afford (1S,2S)-2-fluoro-N-(3-(6-((1-hydroxybutyl)-4-methylpyridin-3-yl)-1-methyl-2-oxo-1,2-dihydro-1,6-naphthyridin-7-yl)cyclopropane-1-carboxamide (retention time 12.99 min, 28.0 mg, 70%) as a white solid and (1S,2S)-2-fluoro-N-(3-(6-(1-hydroxybutyl)-4-methylpyridin-3-yl)-1-methyl-2-oxo-1,2-dihydro-1,6-naphthyridin-7-yl)cyclopropane-1-carboxamide (retention time 16.18, 24.9 mg, 62%) as a white solid.

Isomer A: retention time 1: 12.99 min, LCMS (ESI, m/z): [M+H]⁺=425.2 ¹H NMR (400 MHz, DMSO-d₆): δ 11.19 (s, 1H), 8.72 (s, 1H), 8.27-8.25 (m, 2H), 8.01 (s, 1H), 7.40 (s, 1H), 5.30 (d, J=4.4 Hz, 1H), 5.07-4.89 (m, 1H), 4.60-4.57 (m, 1H), 3.62 (s, 3H), 2.31-2.27 (m, 1H), 2.21 (s, 3H), 1.76-1.62 (m, 3H), 1.43-1.36 (m, 2H), 1.26-1.21 (m, 1H), 0.93-0.89 (m, 3H).

Isomer B: retention time 2: 16.18 min, LCMS (ESI, m/z): [M+H]⁺=425.2 ¹H NMR (400 MHz, DMSO-d₆): δ 11.20 (s, 1H), 8.72 (s, 1H), 8.27-8.25 (m, 2H), 8.01 (s, 1H), 7.40 (s, 1H), 5.30 (d, J=4.8 Hz, 1H), 5.07-4.89 (m, 1H), 4.59-4.57 (m, 1H), 3.62 (s, 3H), 2.34-2.28 (m, 1H), 2.21 (s, 3H), 1.76-1.62 (m, 3H), 1.43-1.36 (m, 2H), 1.26-1.21 (m, 1H), 0.93-0.89 (m, 3H).

The absolute stereochemistry of Isomers A and B was not assigned. The two isomeric structures that could be obtained from chiral separation of the isomeric mixture as described above are shown as Compounds 114 and 115 in Table 1.

### Example S45. Synthesis of (S)—N-(3-(6-(2,2-difluoro-1-hydroxypropyl)-4-methylpyridin-3-yl)-1-methyl-2-oxo-1,2-dihydro-1,6-naphthyridin-7-yl)cyclopropanecarboxamide formate and (R)—N-(3-(6-(2,2-difluoro-1-hydroxypropyl)-4-methylpyridin-3-yl)-1-methyl-2-oxo-1,2-dihydro-1,6-naphthyridin-7-yl)cyclopropanecarboxamide (Compound 116 and 117)

### Step 1. Synthesis of 1-(5-bromo-4-methylpyridin-2-yl)-2-methylprop-2-en-1-ol

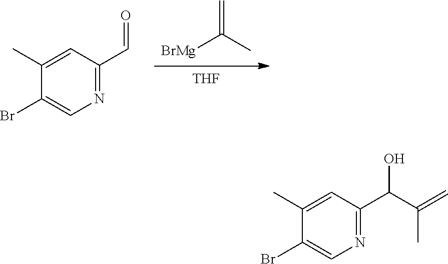

To a solution of 5-bromo-4-methylpicolinaldehyde (10.0 g, 49.99 mmol) in THE (300.0 mL) was added prop-1-en-2-ylmagnesium bromide (120.0 mL, 60.00 mmol, 0.5 M) at 0° C. under N₂. The resulting mixture was stirred at room temperature for 2 h. After the reaction was completed, the resulting mixture was quenched with H₂O and then concentrated under reduced pressure The residue was purified by flash column chromatography with petroleum ether/ethyl acetate (2/1, v/v) to afford 1-(5-bromo-4-methylpyridin-2-yl)-2-methylprop-2-en-1-ol (5.5 g, 45%) as a yellow oil. LCMS (ESI, m/z): [M+H]⁺=242.0.

### Step 2. Synthesis of 1-(5-bromo-4-methylpyridin-2-yl)-2-methylallyl acetate

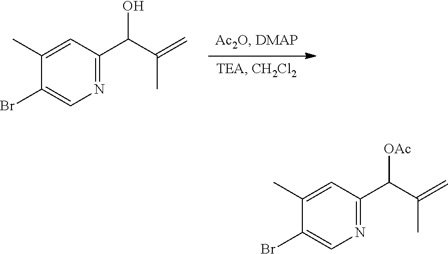

To a solution of 1-(5-bromo-4-methylpyridin-2-yl)-2-methylprop-2-en-1-ol (5.5 g, 22.72 mmol) in DCM (60.0 mL) was added TEA (9.2 g, 90.86 mmol), acetic anhydride (7.0 g, 68.15 mmol) and DMAP (1.4 g, 11.36 mmol) at room temperature. The resulting mixture was stirred at room temperature for 2 h. After the reaction was completed, the mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (5/1, v/v) to afford 1-(5-bromo-4-methylpyridin-2-yl)-2-methylallyl acetate (5.5 g, 85%) as a colorless oil. LCMS (ESI, m/z): [M+H]⁺=284.0.

### Step 3. Synthesis of 1-(5-bromo-4-methylpyridin-2-yl)-2-oxopropyl acetate

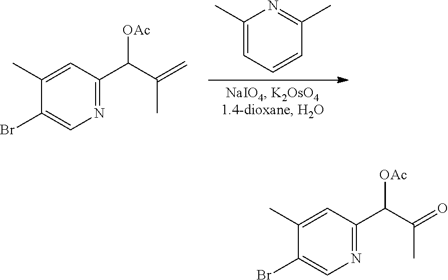

To a solution of 1-(5-bromo-4-methylpyridin-2-yl)-2-methylallyl acetate (5.5 g, 19.36 mmol) in 1.4-dioxane/H₂O (45.0 mL/15.0 mL) was added 2,6-dimethylpyridine (4.2 g, 38.71 mmol), K₂OsO₄·2H₂O in H₂O (5.9 mL, 40%) and NaIO₄ (8.3 g, 38.71 mmol) at 0° C. under N₂. The resulting mixture was stirred at room temperature for 8 h. After the reaction was completed, the resulting mixture was diluted with water and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (5/1, v/v) to afford 1-(5-bromo-4-methylpyridin-2-yl)-2-oxopropyl acetate (3.0 g, 54%) as a yellow oil. LCMS (ESI, m/z): [M+H]⁺=286.0.

### Step 4. Synthesis of 1-(5-bromo-4-methylpyridin-2-yl)-2,2-difluoropropyl acetate

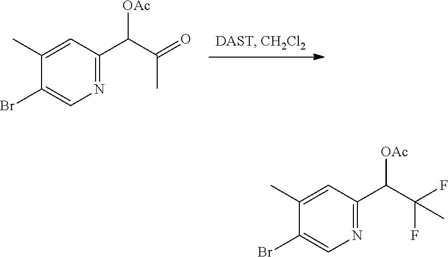

To a solution of 1-(5-bromo-4-methylpyridin-2-yl)-2-oxopropyl acetate (3.0 g, 10.49 mmol) in CH₂Cl₂ (30.0 mL) was added dropwise DAST (3.4 g, 20.50 mmol) at 0° C. under N₂. The resulting mixture was stirred at room temperature for 16 h under N₂. After the reaction was completed, the resulting mixture was quenched with H₂O and extracted with ethyl acetate. The combined organic layer was washed with brine, dried over anhydrous sodium sulfate and filtered. The filtrate was concentrated under reduced pressure. The residue was purified by reverse phase flash column chromatography with H₂O/CH₃CN (1/1, v/v) to afford 1-(5-bromo-4-methylpyridin-2-yl)-2,2-difluoropropyl acetate (1.2 g, 37%) as a yellow oil. LCMS (ESI, m/z): [M+H]⁺=308.0

### Step 5. Synthesis of (6-(1-acetoxy-2,2-difluoropropyl)-4-methylpyridin-3-yl)boronic acid

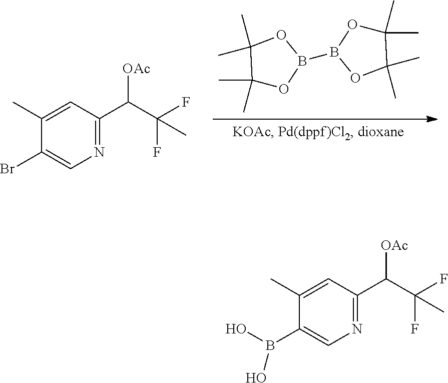

To a solution of 1-(5-bromo-4-methylpyridin-2-yl)-2,2-difluoropropyl acetate (1.2 g, 3.90 mmol) in 1.4-dioxane (20.0 mL) was added 4,4,4′,4′,5,5,5′,5′-octamethyl-2,2′-bi(1,3,2-dioxaborolane) (3.0 g, 11.69 mmol), KOAc (1.2 g, 11.69 mmol) and Pd(dppf)Cl₂ (0.3 g, 0.39 mmol) at room temperature under N₂. The resulting mixture was stirred at 80° C. for 16 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by reverse phase flash column chromatography with H₂O/CH₃CN (1/1, v/v) to afford (6-(1-acetoxy-2,2-difluoropropyl)-4-methylpyridin-3-yl)boronic acid (600.0 mg, 56%) as a yellow oil. LCMS (ESI, m/z): [M+H]⁺=274.1.

### Step 6. Synthesis of 1-(5-(7-chloro-1-methyl-2-oxo-1,2-dihydro-1,6-naphthyridin-3-yl)-4-methylpyridin-2-yl)-2,2-difluoropropyl acetate

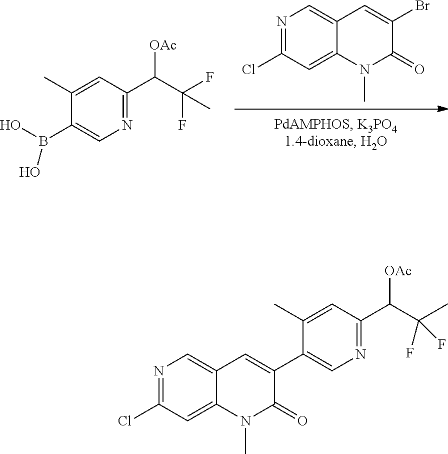

To a solution of (6-(1-acetoxy-2,2-difluoropropyl)-4-methylpyridin-3-yl)boronic acid (600.0 mg, 2.20 mmol) in 1.4-dioxane/H₂O (16.0 mL/4.0 mL) was added 3-bromo-7-chloro-1-methyl-1,6-naphthyridin-2(1H)-one (601.0 mg, 2.20 mmol), K₃PO₄ (1.4 g, 6.59 mmol) and PdAMPHOS (155.6 mg, 0.22 mmol) at room temperature under N₂. The resulting mixture was stirred at 100° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford 1-(5-(7-chloro-1-methyl-2-oxo-1,2-dihydro-1,6-naphthyridin-3-yl)-4-methylpyridin-2-yl)-2,2-difluoropropyl acetate (200.0 mg, 21%) as a yellow oil. LCMS (ESI, m/z): [M+H]⁺=422.1.

### Step 7. Synthesis of N-(3-(6-(2,2-difluoro-1-hydroxypropyl)-4-methylpyridin-3-yl)-1-methyl-2-oxo-1,2-dihydro-1,6-naphthyridin-7-yl)cyclopropanecarboxamide

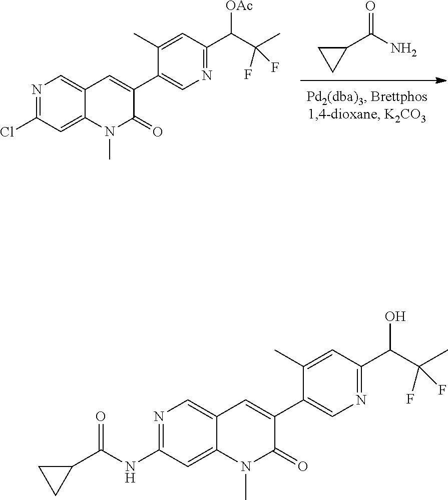

To a solution of 1-(5-(7-chloro-1-methyl-2-oxo-1,2-dihydro-1,6-naphthyridin-3-yl)-4-methylpyridin-2-yl)-2,2-difluoropropyl acetate (140.0 mg, 0.33 mmol) in 1.4-dioxane (5.0 mL) was added cyclopropanecarboxamide (141.2 mg, 1.66 mmol), Brettphos (35.6 mg, 0.07 mmol), K₂CO₃ (137.6 mg, 1.00 mmol) and Pd₂(dba)₃ (30.4 mg, 0.03 mmol) at room temperature under N₂. The resulting mixture was stirred at 100° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/CH₃OH (10/1, v/v) to afford N-(3-(6-(2,2-difluoro-1-hydroxypropyl)-4-methylpyridin-3-yl)-1-methyl-2-oxo-1,2-dihydro-1,6-naphthyridin-7-yl)cyclopropanecarboxamide (50.0 mg, 32%) as a yellow solid. LCMS (ESI, m/z): [M+H]⁺=429.2.

### Step 8. Chiral Separation of (S)—N-(3-(6-(2,2-difluoro-1-hydroxypropyl)-4-methylpyridin-3-yl)-1-methyl-2-oxo-1,2-dihydro-1,6-naphthyridin-7-yl)cyclopropanecarboxamide formate and (R)—N-(3-(6-(2,2-difluoro-1-hydroxypropyl)-4-methylpyridin-3-yl)-1-methyl-2-oxo-1,2-dihydro-1,6-naphthyridin-7-yl)cyclopropanecarboxamide (Compound 116 and 117)

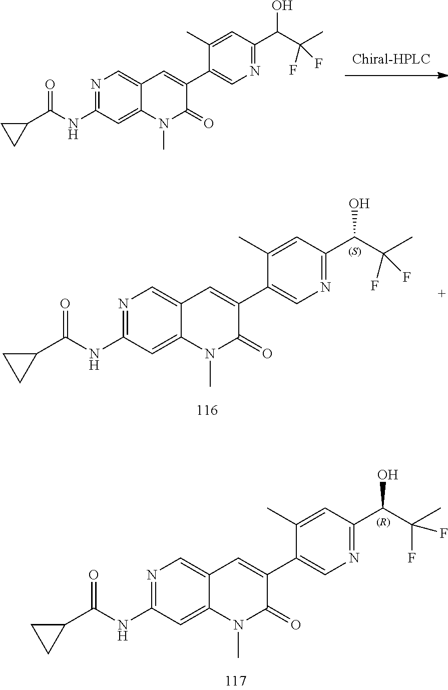

The racemic mixture of N-(3-(6-(2,2-difluoro-1-hydroxypropyl)-4-methylpyridin-3-yl)-1-methyl-2-oxo-1,2-dihydro-1,6-naphthyridin-7-yl)cyclopropanecarboxamide (50.0 mg, 0.36 mmol) was separated by Prep-Chiral-HPLC with the following conditions (Column: CHIRAL ART Amylose-SA, 2×25 cm, 5 m; Mobile Phase A: MtBE (0.1% FA)-HPLC, Mobile Phase B: EtOH-HPLC; Flow rate: 20 mL/min; Gradient: 5% B to 5% B in 15 min; Wave Length: 220/254 nm; RT1 (min): 11.00; RT2 (min): 13.57) to afford (S)—N-(3-(6-(2,2-difluoro-1-hydroxypropyl)-4-methylpyridin-3-yl)-1-methyl-2-oxo-1,2-dihydro-1,6-naphthyridin-7-yl)cyclopropanecarboxamide Enantiomer A (retention time 11.00 min, 15.7 mg, 62%) as a white solid and (R)—N-(3-(6-(2,2-difluoro-1-hydroxypropyl)-4-methylpyridin-3-yl)-1-methyl-2-oxo-1,2-dihydro-1,6-naphthyridin-7-yl)cyclopropanecarboxamide Enantiomer B (retention time 13.5, 18.7 mg, 74%) as a white solid.

Enantiomer A: retention time 1: 11.00 min, LCMS (ESI, m/z): [M+H]⁺=429.1. ¹H NMR (400 MHz, DMSO-d₆): δ 11.16 (s, 1H), 8.71 (s, 1H), 8.34 (s, 1H), 8.26 (s, 1H), 8.13 (s, 1H), 8.04 (s, 1H), 7.47 (s, 1H), 6.40-6.25 (m, 1H), 4.83-4.78 (m, 1H), 3.60 (s, 3H), 2.23 (s, 3H), 2.13-2.07 (m, 1H), 1.69-1.53 (m, 3H), 0.95-0.85 (m, 4H).

Enantiomer B: retention time 2: 13.57 min, LCMS (ESI, m/z): [M+H]⁺=429.1. ¹H NMR (400 MHz, DMSO-d₆): δ 11.16 (s, 1H), 8.71 (s, 1H), 8.34 (s, 1H), 8.26 (s, 1H), 8.04 (s, 1H), 7.47 (s, 1H), 6.34-6.32 (m, 1H), 4.83-4.77 (m, 1H), 3.60 (s, 3H), 2.23 (s, 3H), 2.13-2.06 (m, 1H), 1.69-1.60 (m, 3H), 0.92-0.84 (m, 4H).

The absolute stereochemistry of enantiomers A and B was not assigned. The two enantiomeric structures that could be obtained from chiral separation of the enantiomeric mixture as described above are shown as Compounds 116 and 117 in Table 1.

### Example S46. Synthesis of Atropisomers of (R)—N-(3-(4-(1-hydroxypropyl)-2,6-dimethylphenyl)-1-methyl-2-oxo-1,2-dihydro-1,6-naphthyridin-7-yl)cyclopropanecarboxamide and (S)—N-(3-(4-(1-hydroxypropyl)-2,6-dimethylphenyl)-1-methyl-2-oxo-1,2-dihydro-1,6-naphthyridin-7-yl)cyclopropanecarboxamide (Compound 118, Compound 119, Compound 120 and Compound 121)

### Step 1. Synthesis of Methyl 5-amino-4,6-dibromopyridine-2-carboxylate

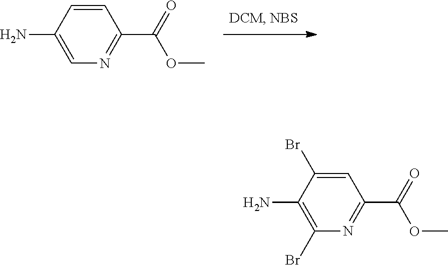

To a solution of methyl 5-aminopyridine-2-carboxylate (40.0 g, 262.98 mmol) in DCM (1000.0 mL) was added NBS (143.9 g, 808.40 mmol) at room temperature. The mixture was stirred at room temperature for 16 h. After the reaction was completed, the mixture was diluted with H₂O and extracted with DCM. The combined organic layer was washed with brine, dried over anhydrous sodium sulfate and filtered. The filtrate was concentrated under reduced pressure. The residue was purified by flash column chromatography with petroleum ether/ethyl acetate (2/1, v/v) to afford methyl 5-amino-4,6-dibromopyridine-2-carboxylate (80.0 g, 95%) as a yellow solid. LCMS (ESI, m/z): [M+H]⁺=308.9.

### Step 2. Synthesis of Methyl 5-amino-4,6-dimethylpyridine-2-carboxylate

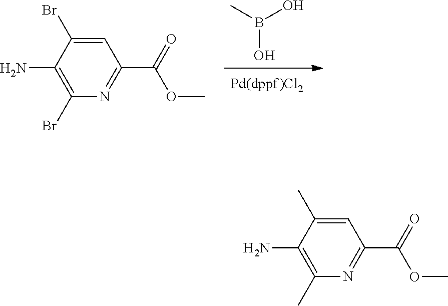

To a solution of methyl 5-amino-4,6-dibromopyridine-2-carboxylate (40.0 g, 129.91 mmol) in dioxane/H₂O (1000.0 mL/100.0 mL) was added methylboronic acid (77.3 g, 1290.55 mmol, 10 eq.), K₂CO₃ (107.0 g, 774.33 mmol) and Pd(dppf)Cl₂ (9.4 g, 12.91 mmol) at room temperature under N₂. The resulting mixture was stirred at 100° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (2/1, v/v) to afford methyl 5-amino-4,6-dimethylpyridine-2-carboxylate (2.5 g, 10%) as a yellow solid. LCMS (ESI, m/z): [M+H]⁺=181.1.

### Step 3. Synthesis of Methyl 5-bromo-4,6-dimethylpyridine-2-carboxylate

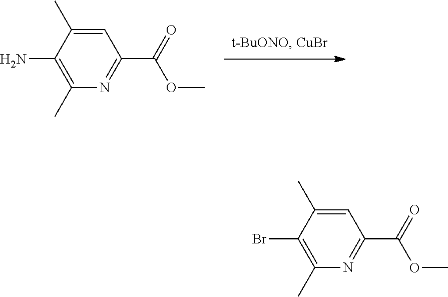

To a solution of methyl 5-amino-4,6-dimethylpyridine-2-carboxylate (5.0 g, 27.75 mmol) in ACN (50.0 mL) was added t-BuONO (8.6 g, 83.24 mmol) and CuBr (11.9 g, 83.24 mmol) at 0° C. under N₂. The resulting mixture was stirred at room temperature for 16 h. After the reaction was completed, the mixture was diluted with H₂O and extracted with CH₂Cl₂. The combined organic layer was washed with brine, dried over anhydrous sodium sulfate and filtered. The filtrate was concentrated under vacuum. The residue was purified by flash column chromatography with petroleum ether/ethyl acetate (2/1, v/v) to afford methyl 5-bromo-4,6-dimethylpyridine-2-carboxylate (4.0 g, 59%) as an off-white solid. LCMS (ESI, m/z): [M+H]⁺=244.0.

### Step 4. Synthesis of 5-bromo-4,6-dimethylpyridine-2-carboxylic acid

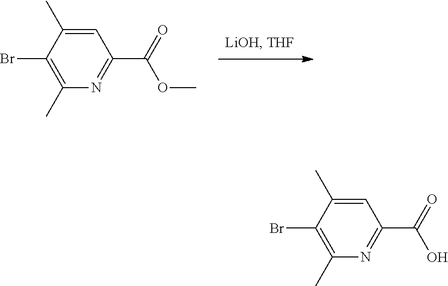

To a mixture of methyl 5-bromo-4,6-dimethylpyridine-2-carboxylate (4.0 g, 16.39 mmol) in THF (40.0 mL)/H₂O (8.0 mL) was added LiOH (1.2 g, 49.16 mmol) at room temperature. The resulting mixture was stirred at room temperature for 16 h. After the reaction was completed, the mixture was basified to pH 7 with HCl (1.0 mol/L). The mixture was concentrated under reduced pressure. The residue was purified by flash column chromatography with CH₂Cl₂/MeOH (10/1, v/v) to afford 5-bromo-4,6-dimethylpyridine-2-carboxylic acid (3.5 g, 92%) as an off-white solid. LCMS (ESI, m/z): [M+H]⁺=230.0.

### Step 5. Synthesis of 5-bromo-N-methoxy-N,4,6-trimethylpyridine-2-carboxamide

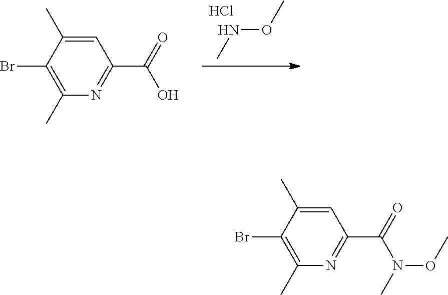

To a solution of 5-bromo-4,6-dimethylpyridine-2-carboxylic acid (3.5 g, 15.21 mmol) in DMF (40.0 mL) was added N,O-dimethylhydroxylamine hydrochloride (2.2 g, 22.82 mmol) and DIEA (7.8 g, 60.85 mmol) at 0° C. under N₂. Then HATU (8.68 g, 22.82 mmol) was added to the mixture at 0° C. The resulting mixture was stirred at room temperature for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (3/1, v/v) to afford 5-bromo-N-methoxy-N,4,6-trimethylpyridine-2-carboxamide (2.5 g, 60%) as a white solid. LCMS (ESI, m/z): [M+H]⁺=273.0.

### Step 6. Synthesis of 1-(5-bromo-4,6-dimethylpyridin-2-yl)propan-1-one

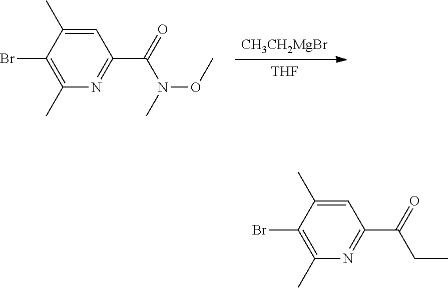

To a solution of 5-bromo-N-methoxy-N,4,6-trimethylpyridine-2-carboxamide (2.5 g, 9.15 mmol) in THE (30.0 mL) was added ethylmagnesium bromide (18.4 mL, 1.0 mmol/L) at 0° C. The resulting mixture was stirred at room temperature for 2 h. After the reaction was completed, the reaction mixture was quenched with sat. NH₄Cl (aq.) at 0° C. The reaction mixture was diluted with H₂O and then extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (2/1, v/v) to afford 1-(5-bromo-4,6-dimethylpyridin-2-yl)propan-1-one (1.8 g, 81%) as a yellow solid. LCMS (ESI, m/z): [M+H]⁺=242.0.

### Step 7. Synthesis of (2,4-dimethyl-6-propionylpyridin-3-yl)boronic acid

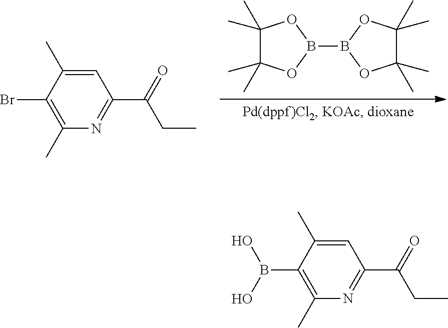

To a solution of 1-(5-bromo-4,6-dimethylpyridin-2-yl)propan-1-one (1.8 g, 7.43 mmol) in dioxane (20.0 mL) was added 4,4,4′,4′,5,5,5′,5′-octamethyl-2,2′-bi(1,3,2-dioxaborolane) (7.1 g, 29.74 mmol), KOAc (2.2 g, 22.30 mmol) and Pd(dppf)Cl₂ (0.5 g, 0.74 mmol) at room temperature under N₂. The resulting mixture was stirred at 100° C. for 16 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford (2,4-dimethyl-6-propionylpyridin-3-yl)boronic acid (800.0 mg, 51%) as a white solid. LCMS (ESI, m/z): [M+H]⁺=208.1.

### Step 8. Synthesis of 7-chloro-3-(2,4-dimethyl-6-propanoylpyridin-3-yl)-1-methyl-1,6-naphthyridin-2-one

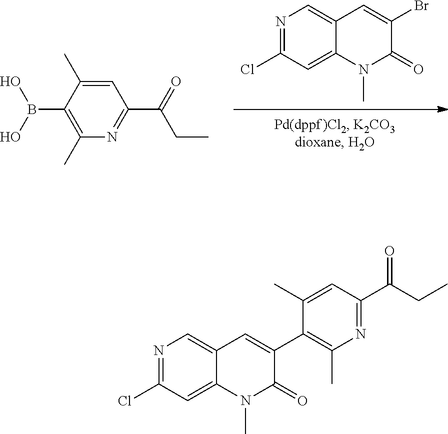 [SMILES: CCC(=O)c1cc(C)c(B(O)O)c(C)n1 ; Cn1c(=O)c(Br)cc2cnc(Cl)cc21 ; CCC(=O)c1cc(C)c(-c2cc3cnc(Cl)cc3n(C)c2=O)c(C)n1]

To a solution of (2,4-dimethyl-6-propionylpyridin-3-yl)boronic acid (800.0 mg, 3.86 mmo) in dioxane (10.0 mL)/H₂O (2.0 mL) were added 3-bromo-7-chloro-1-methyl-1,6-naphthyridin-2-one (1.1 g, 3.86 mmol), K₂CO₃ (1.6 g, 11.58 mmol) and Pd(dppf)Cl₂ (282.6 mg, 0.38 mmol) at room temperature under N₂. The resulting mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/MeOH (10/1, v/v) to afford 7-chloro-3-(2,4-dimethyl-6-propanoylpyridin-3-yl)-1-methyl-1,6-naphthyridin-2-one (450.0 mg, 45%) as a white solid. LCMS (ESI, m/z): [M+H]⁺=356.1.

### Step 9. Synthesis of N-[3-(2,4-dimethyl-6-propanoylpyridin-3-yl)-1-methyl-2-oxo-1,6-naphthyridin-7-yl]cyclopropanecarboxamide

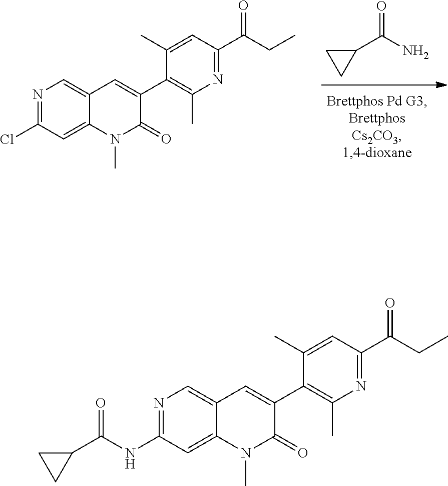

To a solution of 7-chloro-3-(2,4-dimethyl-6-propanoylpyridin-3-yl)-1-methyl-1,6-naphthyridin-2-one (450.0 mg, 1.26 mmol) in dioxane (10.0 mL) were added cyclopropanecarboxamide (215.3 mg, 2.53 mmol), Cs₂CO₃ (1.2 g, 3.79 mmol), BrettPhos (135.7 mg, 0.25 mmol) and BrettPhos Pd G3 (114.6 mg, 0.13 mmol) at room temperature under N₂. The resulting mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/MeOH (10/1, v/v) to afford N-[3-(2,4-dimethyl-6-propanoylpyridin-3-yl)-1-methyl-2-oxo-1,6-naphthyridin-7-yl]cyclopropanecarboxamide (260.0 mg, 50%) as a white solid. LCMS (ESI, m/z): [M+H]⁺=405.2.

### Step 10. Synthesis of N-{3-[6-(1-hydroxypropyl)-2,4-dimethylpyridin-3-yl]-1-methyl-2-oxo-1,6-naphthyridin-7-yl}cyclopropanecarboxamide

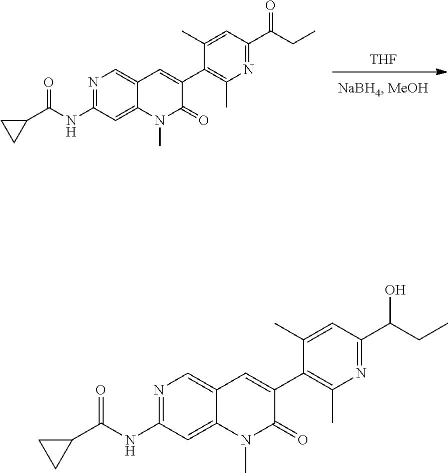 [SMILES: CCC(=O)c1cc(C)c(-c2cc3cnc(NC(=O)C4CC4)cc3n(C)c2=O)c(C)n1 ; CCC(O)c1cc(C)c(-c2cc3cnc(NC(=O)C4CC4)cc3n(C)c2=O)c(C)n1 ; "THF"]

To a solution of N-[3-(2,4-dimethyl-6-propanoylpyridin-3-yl)-1-methyl-2-oxo-1,6-naphthyridin-7-yl]cyclopropanecarboxamide (260.0 mg, 0.64 mmol) in THF (10.0 mL)/MeOH (1.0 mL) were added NaBH₄ (121.6 mg, 3.22 mmol) at 0° C. The resulting mixture was stirred at room temperature for 2 h. After the reaction was completed, the reaction mixture was diluted with H₂O and then extracted with ethyl acetate. The combined organic layer was washed with brine, dried over anhydrous sodium sulfate and filtered. The filtrate was concentrated under reduced pressure. The residue was purified by flash column chromatography with CH₂Cl₂/MeOH (10/1, v/v) to afford N-{3-[6-(1-hydroxypropyl)-2,4-dimethylpyridin-3-yl]-1-methyl-2-oxo-1,6-naphthyridin-7-yl}cyclopropanecarboxamide (200.0 mg, 76%) as a white solid. LCMS (ESI, m/z): [M+H]⁺=407.2.

### Step 11. Chiral Separation of (R)—N-(3-(4-(1-hydroxypropyl)-2,6-dimethylphenyl)-1-methyl-2-oxo-1,2-dihydro-1,6-naphthyridin-7-yl)cyclopropanecarboxamide & (R)—N-(3-(4-(1-hydroxypropyl)-2,6-dimethylphenyl)-1-methyl-2-oxo-1,2-dihydro-1,6-naphthyridin-7-yl)cyclopropanecarboxamide & (S)—N-(3-(4-(1-hydroxypropyl)-2,6-dimethylphenyl)-1-methyl-2-oxo-1,2-dihydro-1,6-naphthyridin-7-yl)cyclopropanecarboxamide & (S)—N-(3-(4-(1-hydroxypropyl)-2,6-dimethylphenyl)-1-methyl-2-oxo-1,2-dihydro-1,6-naphthyridin-7-yl)cyclopropanecarboxamide (Compound 118, Compound 119, Compound 120 and Compound 121)

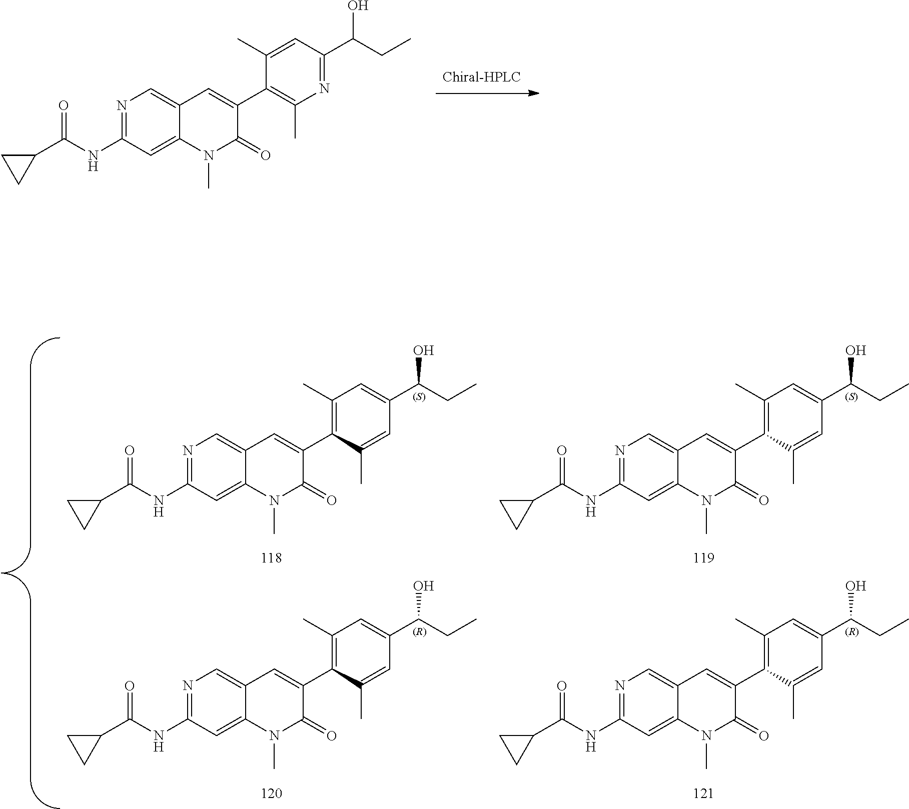

The diastereomeric mixture of N-{3-[6-(1-hydroxypropyl)-2,4-dimethylpyridin-3-yl]-1-methyl-2-oxo-1,6-naphthyridin-7-yl}cyclopropanecarboxamide (100.0 mg, 0.25 mmol) was separated by Prep-chiral-HPLC with the following the conditions: (Column: CHIRAL ART Cellulose-SC, 2×25 cm, 5 m; Mobile Phase A: MtBE (0.5% 2M NH₃-MeOH)-HPLC, Mobile Phase B: MeOH:DCM=1:1-HPLC; Flow rate: 20 mL/min; Gradient: 20% B to 20% B in 13.5 min; Wave Length: 220/254 nm; RT1(min): 7.30; RT2(min): 9.27) to afford Isomer A (1^{st} Chiral HPLC retention time 7.30 min, 15.2 mg, 60%) as a white solid, Isomer B (1^{st} Chiral HPLC retention time 9.27 min, 14.0 mg, 56%) as a white solid and the mixture of Isomer C and Isomer D (50.0 mg, 50%) as a white solid. The mixture of Isomer C and Isomer D was purified by Prep-CHIRAL-HPLC again with the following conditions: (Column: CHIRAL ART Cellulose-SC, 2×25 cm, 5 m; Mobile Phase A: Hex (0.5% 2M NH₃-MeOH)-HPLC, Mobile Phase B: MeOH:DCM=1:1-HPLC; Flow rate: 20 mL/min; Gradient: 40% B to 40% B in 11.5 min; Wave Length: 220/254 nm; RT1(min): 8.31; RT2(min): 10.64) to afford Isomer C (2^{nd} Chiral HPLC retention time 8.31 min, 14.2 mg, 56%) as a white solid and Isomer D ((2^{nd} Chiral HPLC retention time 10.64 min, 15.1 mg, 60%) as a white solid.

Isomer A: retention time 1 (first Chiral HPLC): 7.30 min, LCMS (ESI, m/z): [M+H]⁺=407.2. ¹H NMR (400 MHz, DMSO-d₆): δ 11.17 (s, 1H), 8.66 (s, 1H), 8.26 (s, 1H), 7.94 (s, 1H), 7.25 (s, 1H), 5.28 (d, J=4.8 Hz, 1H), 4.48-4.44 (m, 1H), 3.60 (s, 3H), 2.22 (s, 3H), 2.12-2.08 (m, 4H), 1.84-1.77 (m, 1H), 1.64-1.55 (m, 1H), 0.93-0.84 (m, 7H).

Isomer B: retention time 2 (first Chiral HPLC): 9.27 min, LCMS (ESI, m/z): [M+H]⁺=407.1. ¹H NMR (400 MHz, DMSO-d₆): δ 11.16 (s, 1H), 8.66 (s, 1H), 8.26 (s, 1H), 7.94 (s, 1H), 7.24 (s, 1H), 5.26 (d, J=4.8 Hz, 1H), 4.48-4.44 (m, 1H), 3.60 (s, 3H), 2.22 (s, 3H), 2.12-2.08 (m, 4H), 1.84-1.77 (m, 1H), 1.65-1.58 (m, 1H), 0.93-0.84 (m, 7H).

Isomer C: retention time 1 (second Chiral HPLC): 8.31 min, LCMS (ESI, m/z): [M+H]⁺=407.2. ¹H NMR (400 MHz, DMSO-d₆): δ 11.15 (s, 1H), 8.68 (s, 1H), 8.26 (s, 1H), 7.89 (s, 1H), 7.24 (s, 1H), 5.21 (d, J=5.2 Hz, 1H), 4.47-4.43 (m, 1H), 3.60 (s, 3H), 2.22 (s, 3H), 2.12-2.06 (m, 4H), 1.87-1.79 (m, 1H), 1.68-1.57 (m, 1H), 0.94-0.84 (m, 7H).

Isomer D: retention time 2 (second Chiral HPLC): 10.64 min, LCMS (ESI, m/z): [M+H]⁺=407.2. ¹H NMR (400 MHz, DMSO-d₆): δ 11.15 (s, 1H), 8.68 (s, 1H), 8.26 (s, 1H), 7.89 (s, 1H), 7.24 (s, 1H), 5.21 (d, J=5.2 Hz, 1H), 4.47-4.43 (m, 1H), 3.60 (s, 3H), 2.22 (s, 3H), 2.12-2.08 (m, 4H), 1.87-1.78 (m, 1H), 1.68-1.57 (m, 1H), 0.94-0.84 (m, 7H)..

The absolute stereochemistry of Isomers A, B, C and D was not assigned. The four atropisomers of the two diastereomeric structures that could be obtained from chiral separation of the isomeric mixture as described above are shown as Compounds 118, 119, 120 and 121 in Table 1.

### Example S47. Synthesis of N-{1-methyl-3-[4-methyl-6-(pyrazol-1-yl)pyridin-3-yl]-2-oxo-1,6-naphthyridin-7-yl}cyclopropanecarboxamide (Compound 122)

### Step 1. Synthesis of 5-bromo-4-methyl-2-(pyrazol-1-yl)pyridine

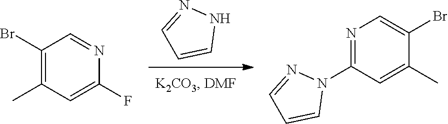

To a mixture of 5-bromo-2-fluoro-4-methylpyridine (5.0 g, 26.31 mmol) in DMF (50.0 mL) was added pyrazole (1.79 g, 26.31 mmol) and K₂CO₃ (10.9 g, 78.94 mmol) at room temperature. The resulting mixture was stirred at 110° C. for 16 h. After the reaction was completed, the resulting mixture was diluted with H₂O and extracted with ethyl acetate. The combined organic layer was washed with brine, dried over anhydrous sodium sulfate and filtered. The filtrate was evaporated in vacuo. The residue was purified by flash column chromatography with CH₂Cl₂/MeOH (10/1, v/v) to afford 5-bromo-4-methyl-2-(pyrazol-1-yl)pyridine (5.0 g, 79%) as an off-white solid. LCMS (ESI, m/z): [M+H]⁺=238.0.

### Step 2. Synthesis of 4-methyl-6-(pyrazol-1-yl)pyridin-3-ylboronic acid

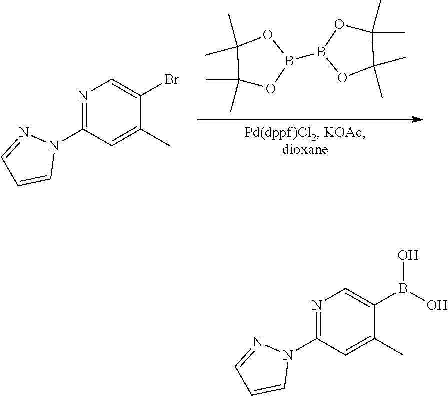

To a solution of 5-bromo-4-methyl-2-(pyrazol-1-yl)pyridine (1.0 g, 4.2 mmol) in dioxane (20.0 mL) was added 4,4,4′,4′,5,5,5′,5′-octamethyl-2,2′-bi(1,3,2-dioxaborolane) (1.2 g, 5.04 mmol), KOAc (1.2 g, 12.6 mmol) and Pd(dppf)Cl₂ (0.3 g, 0.42 mmol) at room temperature under N₂. The resulting mixture was stirred at 100° C. for 2 h. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/MeOH (10/1, v/v) to afford 4-methyl-6-(pyrazol-1-yl)pyridin-3-ylboronic acid (800.0 mg, 93%) as a light blue solid. LCMS (ESI, m/z): [M+H]⁺=204.1.

### Step 3. Synthesis of 7-chloro-1-methyl-3-[4-methyl-6-(pyrazol-1-yl)pyridin-3-yl]-1,6-naphthyridin-2-one

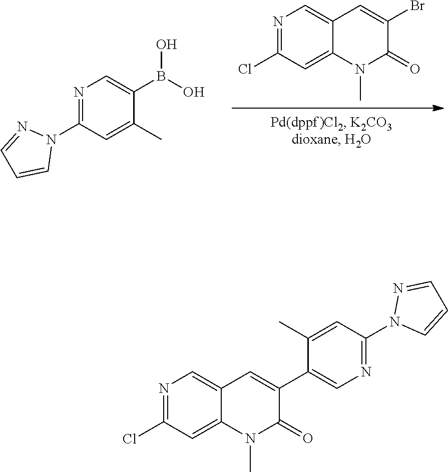 [SMILES: Cc1cc(-n2cccn2)ncc1B(O)O ; Cn1c(=O)c(Br)cc2cnc(Cl)cc21 ; Cc1cc(-n2cccn2)ncc1-c1cc2cnc(Cl)cc2n(C)c1=O]

To a solution of 4-methyl-6-(pyrazol-1-yl)pyridin-3-ylboronic acid (500.0 mg, 2.46 mmol) in 1,4-dioxane (10.0 mL) and H₂O (2.5 mL) were added 3-bromo-7-chloro-1-methyl-1,6-naphthyridin-2-one (673.6 mg, 2.46 mmol), K₂CO₃ (1021.1 mg, 7.38 mmol) and Pd(dppf)Cl₂ (180.2 mg, 0.24 mmol) at room temperature under N₂. The mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/MeOH (10/1, v/v) to afford 7-chloro-1-methyl-3-[4-methyl-6-(pyrazol-1-yl)pyridin-3-yl]-1,6-naphthyridin-2-one (180.0 mg, 20%) as a white solid. LCMS (ESI, m/z): [M+H]⁺=352.1.

### Step 4. Synthesis of N-{1-methyl-3-[4-methyl-6-(pyrazol-1-yl)pyridin-3-yl]-2-oxo-1,6-naphthyridin-7-yl}cyclopropanecarboxamide (Compound 122)

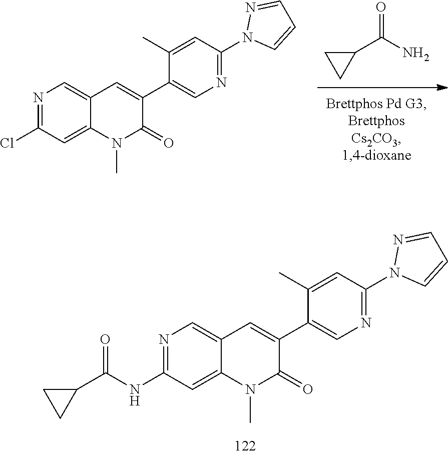

To a solution of 7-chloro-1-methyl-3-[4-methyl-6-(pyrazol-1-yl)pyridin-3-yl]-1,6-naphthyridin-2-one (170.0 mg, 0.48 mmol) in 1,4-dioxane (5.0 mL) was added cyclopropanecarboxamide (41.1 mg, 0.48 mmol), Cs₂CO₃ (472.3 mg, 1.44 mmol), BrettPhos (51.8 mg, 0.09 mmol) and BrettPhos Pd G3 (43.8 mg, 0.04 mmol) at room temperature under N₂. The resulting mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/MeOH (10/1, v/v) and then purified by Prep-HPLC with the following conditions: (Column: XBridge Prep OBD C18 Column, 30×150 mm, 5 m; Mobile Phase A: Water (10 mmol/L NH₄HCO₃), Mobile Phase B: ACN; Flow rate: 60 mL/min; Gradient: 33% B to 38% B in 8 min; Wave Length: 254 nm) to afford N-{1-methyl-3-[4-methyl-6-(pyrazol-1-yl)pyridin-3-yl]-2-oxo-1,6-naphthyridin-7-yl}cyclopropanecarboxamide (Compound 122) (25.8 mg, 13%) as an off-white solid. LCMS (ESI, m/z): [M+H]⁺=401.1. ¹H NMR (400 MHz, DMSO-d₆): δ 11.15 (s, 1H), 8.73 (s, 1H), 8.64 (d, J=2.0 Hz, 1H), 8.29-8.27 (m, 2H), 8.07 (s, 1H), 7.93-7.85 (m, 2H), 6.60 (s, 1H), 3.61 (s, 3H), 2.31 (s, 3H), 2.15-2.07 (m, 1H), 0.92-0.84 (m, 4H).

### Example S48. Synthesis of (1S)-2,2-difluoro-N-(3-{6-[(1R)-1-hydroxypropyl]-4-methylpyridin-3-yl}-1-methyl-2-oxo-1,6-naphthyridin-7-yl)cyclopropane-1-carboxamide and (1S)-2,2-difluoro-N-(3-{6-[(1S)-1-hydroxypropyl]-4-methylpyridin-3-yl}-1-methyl-2-oxo-1,6-naphthyridin-7-yl)cyclopropane-1-carboxamide (Compound 123 and 124)

### Step 1. Synthesis of Tert-butyl N-[1-methyl-3-(4-methyl-6-propanoylpyridin-3-yl)-2-oxo-1,6-naphthyridin-7-yl]carbamate

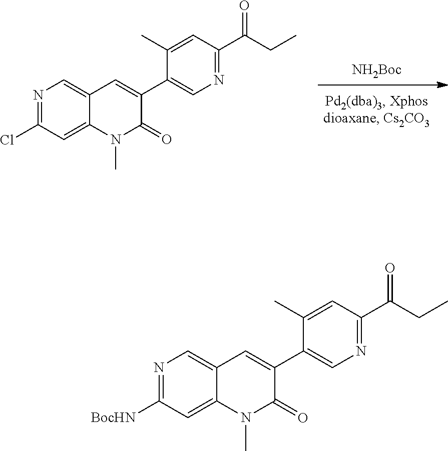 [SMILES: CCC(=O)c1cc(C)c(-c2cc3cnc(Cl)cc3n(C)c2=O)cn1 ; CCC(=O)c1cc(C)c(-c2cc3cnc(NC(=O)OC(C)(C)C)cc3n(C)c2=O)cn1 ; "NH2Boc"]

To a solution of 7-chloro-1-methyl-3-(4-methyl-6-propanoylpyridin-3-yl)-1,6-naphthyridin-2-one (2.2 g, 6.43 mmol) in 1,4-dioxane (20.0 mL) was added tert-butyl carbamate (3.8 g, 32.18 mmol), Pd₂(dba)₃ (290.0 mg, 0.32 mmol), Cs₂CO₃ (3.1 g, 9.66 mmol) and XPhos (0.3 g, 0.644 mmol) at room temperature under N₂. The resulting mixture was stirred at 80° C. for 16 h under N₂. After the reaction was completed, the mixture was filtered. The filtrate was concentrated under reduced pressure. The residue was purified by flash chromatography with DCM/EtOAc (60/40, v/v) to afford tert-butyl N-[1-methyl-3-(4-methyl-6-propanoylpyridin-3-yl)-2-oxo-1,6-naphthyridin-7-yl]carbamate (1.5 g, 44%) as a yellow solid. LCMS (ESI, m/z): [M+H]⁺=423.2.

### Step 2. Synthesis of 7-amino-1-methyl-3-(4-methyl-6-propionylpyridin-3-yl)-1,6-naphthyridin-2(1H)-one hydrochloride

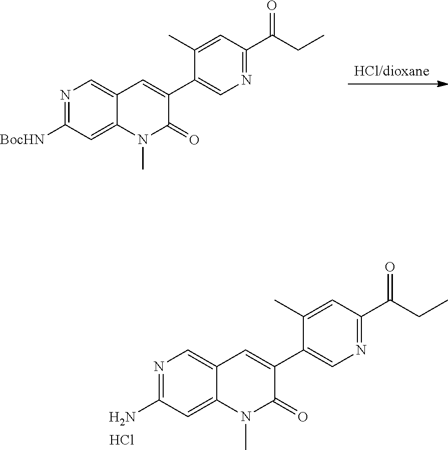 [SMILES: CCC(=O)c1cc(C)c(-c2cc3cnc(NC(=O)OC(C)(C)C)cc3n(C)c2=O)cn1 ; "HCl/dioxane" ; Cl ; CCC(=O)c1cc(C)c(-c2cc3cnc(N)cc3n(C)c2=O)cn1]

A solution of tert-butyl N-[1-methyl-3-(4-methyl-6-propanoylpyridin-3-yl)-2-oxo-1,6-naphthyridin-7-yl]carbamate (500.0 mg, 1.18 mmol) in HCl/1,4-dioxane (10.0 mL, 4.0 mol/L) was stirred at room temperature for 1 h. After the reaction was completed, the resulting mixture was concentrated under vacuum to afford 7-amino-1-methyl-3-(4-methyl-6-propionylpyridin-3-yl)-1,6-naphthyridin-2(1H)-one hydrochloride (540.0 mg, crude) as a yellow solid. LCMS (ESI, m/z): [M+H]⁺=323.1.

### Step 3. Synthesis of (1S)-2,2-difluoro-N-[1-methyl-3-(4-methyl-6-propanoylpyridin-3-yl)-2-oxo-1,6-naphthyridin-7-yl]cyclopropane-1-carboxamide

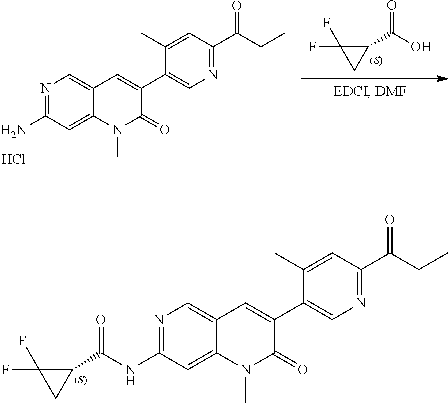

To a solution of 7-amino-1-methyl-3-(4-methyl-6-propionylpyridin-3-yl)-1,6-naphthyridin-2(1H)-one hydrochloride (500.0 mg, crude) in DMF (5.0 mL) was added (1S)-2,2-difluorocyclopropane-1-carboxylic acid (850.4 mg, 6.96 mmol) and EDCI (1335.6 mg, 6.96 mmol) at room temperature. The resulting mixture was stirred at room temperature for 16 h. After the reaction was completed, the mixture was diluted with H₂O and extracted with ethyl acetate. The combined organic layer was washed with brine, dried over anhydrous sodium sulfate and filtered. The filtrate was concentrated under reduced pressure. The residue was purified by reverse phase flash chromatography with MeOH/H₂O (40/60, v/v) to afford (1S)-2,2-difluoro-N-[1-methyl-3-(4-methyl-6-propanoylpyridin-3-yl)-2-oxo-1,6-naphthyridin-7-yl]cyclopropane-1-carboxamide (210.0 mg, 27%) as a yellow solid. LCMS (ESI, m/z): [M+H]⁺=427.2.

### Step 4. Synthesis of (1S)-2,2-difluoro-N-{3-[6-(1-hydroxypropyl)-4-methylpyridin-3-yl]-1-methyl-2-oxo-1,6-naphthyridin-7-yl}cyclopropane-1-carboxamide

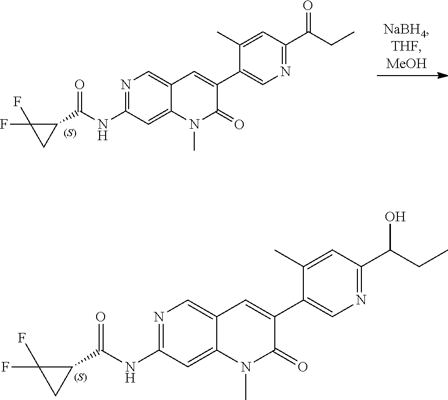

To a solution of (1S)-2,2-difluoro-N-[1-methyl-3-(4-methyl-6-propanoylpyridin-3-yl)-2-oxo-1,6-naphthyridin-7-yl]cyclopropane-1-carboxamide (200.0 mg, 0.46 mmol) in THF (4.0 mL) and MeOH (2.0 mL) was added NaBH₄ (88.7 mg, 2.34 mmol) at room temperature. The resulting mixture was stirred at room temperature for 3 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash chromatography with DCM/MeOH (90/10, v/v) to afford (1S)-2,2-difluoro-N-{3-[6-(1-hydroxypropyl)-4-methylpyridin-3-yl]-1-methyl-2-oxo-1,6-naphthyridin-7-yl}cyclopropane-1-carboxamide (130.0 mg, 58%) as a yellow oil. LCMS (ESI, m/z): [M+H]⁺=429.2.

### Step 5. Chiral Separation of (1S)-2,2-difluoro-N-(3-{6-[(1R)-1-hydroxypropyl]-4-methylpyridin-3-yl}-1-methyl-2-oxo-1,6-naphthyridin-7-yl)cyclopropane-1-carboxamide and (1S)-2,2-difluoro-N-(3-{6-[(1S)-1-hydroxypropyl]-4-methylpyridin-3-yl}-1-methyl-2-oxo-1,6-naphthyridin-7-yl)cyclopropane-1-carboxamide (Compound 123 and 124)

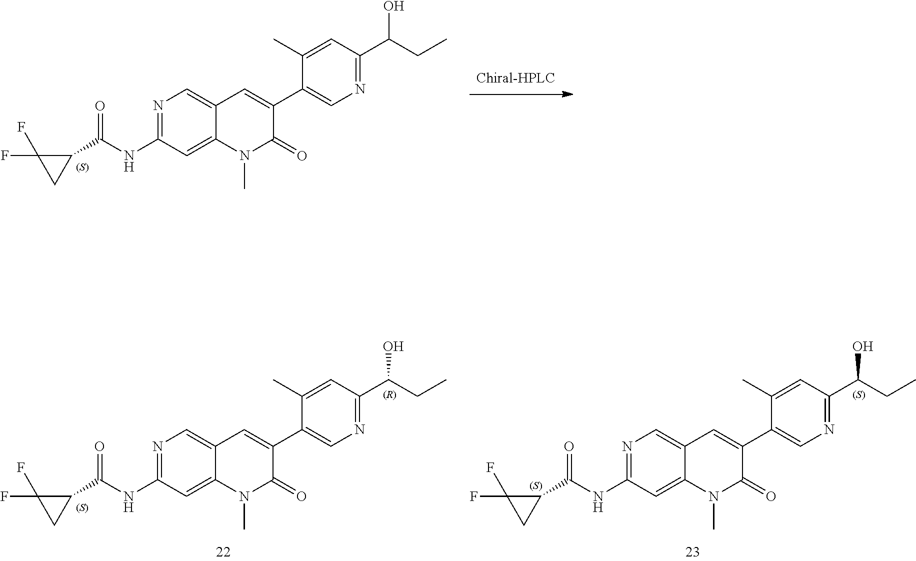

The racemic mixture of (1S)-2,2-difluoro-N-{3-[6-(1-hydroxypropyl)-4-methylpyridin-3-yl]-1-methyl-2-oxo-1,6-naphthyridin-7-yl}cyclopropane-1-carboxamide (110.0 mg, 0.25 mmol) was separated by Prep-Chiral-HPLC with the following conditions (Column: CHIRAL ART Cellulose-SB, 2×25 cm, 5 m; Mobile Phase A: Hex (0.2% TFA)-HPLC, Mobile Phase B: EtOH:DCM=1:1-HPLC; Flow rate: 20 mL/min; Gradient: 30% B to 30% B in 15 min; Wave Length: 220/254 nm; RT1(min): 7.98; RT2(min): 12.73) to afford (1S)-2,2-difluoro-N-(3-{6-[1-hydroxypropyl]-4-methylpyridin-3-yl}-1-methyl-2-oxo-1,6-naphthyridin-7-yl)cyclopropane-1-carboxamide Isomer A (retention time 7.98 min, 42.9 mg, 78%) as a white solid and (1S)-2,2-difluoro-N-(3-{6-[1-hydroxypropyl]-4-methylpyridin-3-yl}-1-methyl-2-oxo-1,6-naphthyridin-7-yl)cyclopropane-1-carboxamide Isomer B (retention time 12.73 min, 46.2 mg, 84%) as a white solid.

Isomer A: retention time 1: 7.98 min, LCMS (ESI, m/z): [M+H]⁺=429.1. 1H NMR (400 MHz, DMSO-d₆): δ 11.39 (s, 1H), 8.77 (s, 1H), 8.55 (s, 1H), 8.24 (s, 1H), 8.15 (s, 1H), 7.83 (s, 1H), 4.81 (d, J=5.2 Hz, 1H), 3.63 (s, 3H), 3.13-3.05 (m, 1H), 2.34 (s, 3H), 2.12-2.06 (m, 2H), 1.91-1.85 (m, 1H), 1.78-1.72 (m, 1H), 0.94-0.88 (m, 3H).

Isomer B: retention time 2: 12.73 min, LCMS (ESI, m/z): [M+H]⁺=429.2. ¹H NMR (400 MHz, DMSO-d₆): δ 11.42 (s, 1H), 8.77 (s, 1H), 8.57 (s, 1H), 8.25 (s, 1H), 8.17 (s, 1H), 7.87 (s, 1H), 4.86-4.82 (m, 1H), 3.63 (s, 3H), 3.13-3.05 (m, 1H), 2.41 (s, 3H), 2.13-2.06 (m, 2H), 1.92-1.85 (m, 1H), 1.80-1.69 (m, 1H), 0.94-0.88 (m, 3H).

The absolute stereochemistry of Isomers A and B was not assigned. The two isomeric structures that could be obtained from chiral separation of the isomeric mixture as described above are shown as Compounds 123 and 124 in Table 1.

### Example S49. Synthesis of (1R)-2,2-difluoro-N-(3-{6-[(1R)-1-hydroxypropyl]-4-methylpyridin-3-yl}-1-methyl-2-oxo-1,6-naphthyridin-7-yl)cyclopropane-1-carboxamide and (1R)-2,2-difluoro-N-(3-{6-[(1S)-1-hydroxypropyl]-4-methylpyridin-3-yl}-1-methyl-2-oxo-1,6-naphthyridin-7-yl)cyclopropane-1-carboxamide (Compound 125 and 126)

### Step 1. Synthesis of (1R)-2,2-difluoro-N-[1-methyl-3-(4-methyl-6-propanoylpyridin-3-yl)-2-oxo-1,6-naphthyridin-7-yl]cyclopropane-1-carboxamide

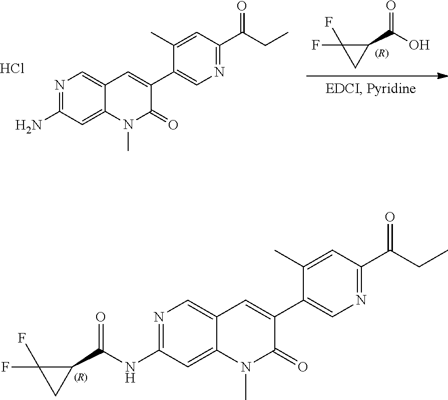

To a solution of 7-amino-1-methyl-3-(4-methyl-6-propanoylpyridin-3-yl)-1,6-naphthyridin-2-one hydrochloride (550.0 mg, crude) in Pyridine (15.0 mL) was added (1R)-2,2-difluorocyclopropane-1-carboxylic acid (935.5 mg, 7.66 mmol) and EDCI (1469.1 mg, 7.66 mmol) at room temperature. The resulting mixture was stirred at room temperature for 16 h. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure. The residue was purified by flash chromatography with CH₂Cl₂/MeOH (90/10, v/v) to afford (1R)-2,2-difluoro-N-[1-methyl-3-(4-methyl-6-propanoylpyridin-3-yl)-2-oxo-1,6-naphthyridin-7-yl]cyclopropane-1-carboxamide (600.0 mg, 73%) as a yellow solid. LCMS (ESI, m/z): [M+H]⁺=427.2.

### Step 2. Synthesis of (1R)-2,2-difluoro-N-{3-[6-(1-hydroxypropyl)-4-methylpyridin-3-yl]-1-methyl-2-oxo-1,6-naphthyridin-7-yl}cyclopropane-1-carboxamide

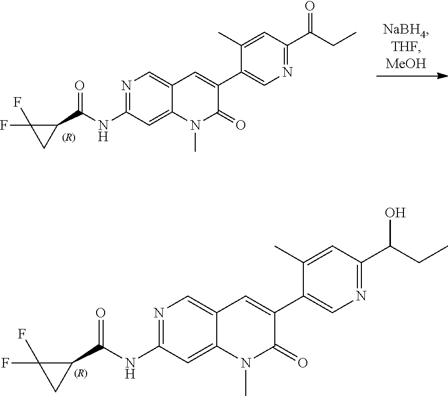

To a solution of (1R)-2,2-difluoro-N-[1-methyl-3-(4-methyl-6-propanoylpyridin-3-yl)-2-oxo-1,6-naphthyridin-7-yl]cyclopropane-1-carboxamide (600.0 mg, 1.40 mmol) in THE (5.0 mL) and MeOH (5.0 mL) was added NaBH₄ (266.1 mg, 7.03 mmol) at room temperature. The resulting mixture was stirred at room temperature for 3 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash chromatography with DCM/MeOH (90/10, v/v) to afford (1R)-2,2-difluoro-N-{3-[6-(1-hydroxypropyl)-4-methylpyridin-3-yl]-1-methyl-2-oxo-1,6-naphthyridin-7-yl}cyclopropane-1-carboxamide (200.0 mg, 33%) as an off-white solid. LCMS (ESI, m/z): [M+H]⁺=429.2.

### Step 3. Chiral Separation of (1R)-2,2-difluoro-N-(3-{6-[(1R)-1-hydroxypropyl]-4-methylpyridin-3-yl}-1-methyl-2-oxo-1,6-naphthyridin-7-yl)cyclopropane-1-carboxamide and (1R)-2,2-difluoro-N-(3-{6-[(1S)-1-hydroxypropyl]-4-methylpyridin-3-yl}-1-methyl-2-oxo-1,6-naphthyridin-7-yl)cyclopropane-1-carboxamide (Compound 125 and 126)

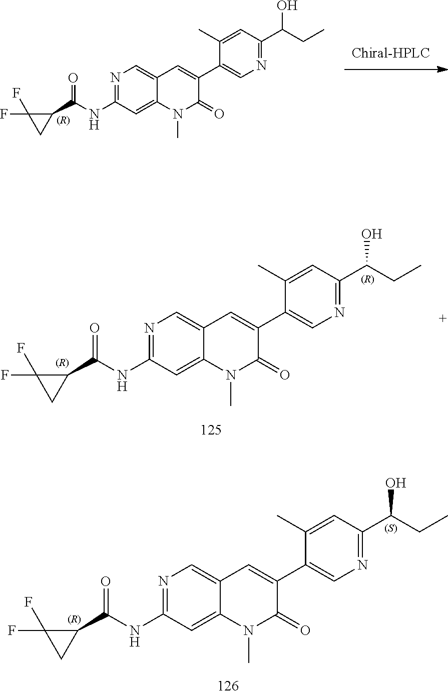

The racemic mixture of (1R)-2,2-difluoro-N-{3-[6-(1-hydroxypropyl)-4-methylpyridin-3-yl]-1-methyl-2-oxo-1,6-naphthyridin-7-yl}cyclopropane-1-carboxamide (110.0 mg, 0.25 mmol) was separated by Prep-Chiral-HPLC with the following conditions (Column: CHIRAL ART Cellulose-SB, 2×25 cm, 5 m; Mobile Phase A: Hex (0.1% TFA)-HPLC, Mobile Phase B: EtOH:DCM=1:1-HPLC; Flow rate: 20 mL/min; Gradient: 30% B to 30% B in 15 min; Wave Length: 220/254 nm; RT1(min): 9.43; RT2(min): 11.94) to afford (1R)-2,2-difluoro-N-(3-{6-[1-hydroxypropyl]-4-methylpyridin-3-yl}-1-methyl-2-oxo-1,6-naphthyridin-7-yl)cyclopropane-1-carboxamide Isomer A (retention time 9.43 min, 50.0 mg, crude) as a white solid and (1R)-2,2-difluoro-N-(3-{6-[1-hydroxypropyl]-4-methylpyridin-3-yl}-1-methyl-2-oxo-1,6-naphthyridin-7-yl)cyclopropane-1-carboxamide Isomer B (retention time 11.94 min, 75.0 mg, crude) as a white solid.

Isomer A: The crude of (1R)-2,2-difluoro-N-(3-{6-[1-hydroxypropyl]-4-methylpyridin-3-yl}-1-methyl-2-oxo-1,6-naphthyridin-7-yl)cyclopropane-1-carboxamide Isomer A (retention time 9.43 min, 50.0 mg, crude) was purified by Prep-HPLC with the following conditions (Column: XSelect CSH Prep C18 OBD Column, 19×250 mm, 5 m; Mobile Phase A: Water (0.05% FA), Mobile Phase B: ACN; Flow rate: 25 mL/min; Gradient: 24% B to 29% B in 8 min, 29% B; Wave Length: 220 nm) to afford (1R)-2,2-difluoro-N-(3-{6-[1-hydroxypropyl]-4-methylpyridin-3-yl}-1-methyl-2-oxo-1,6-naphthyridin-7-yl)cyclopropane-1-carboxamide Isomer A (32.6 mg, 59%) as a white solid. RT1(min): 9.43; LCMS (ESI, m/z): [M+H]⁺=429.1. ¹H NMR (400 MHz, DMSO-d₆): δ 11.40 (s, 1H), 8.77 (d, J=7.2 Hz, 1H), 8.53 (s, 1H), 8.24 (s, 1H), 8.14 (s, 1H), 7.79 (s, 1H), 4.82-4.77 (m, 1H), 3.63 (s, 3H), 3.13-3.04 (m, 1H), 2.38 (s, 3H), 2.12-2.05 (m, 2H), 1.90-1.82 (m, 1H), 1.76-1.73 (m, 1H), 0.99-0.83 (m, 3H).

Isomer B: The crude of (1R)-2,2-difluoro-N-(3-{6-[(1-hydroxypropyl]-4-methylpyridin-3-yl}-1-methyl-2-oxo-1,6-naphthyridin-7-yl)cyclopropane-1-carboxamide Isomer B (retention time 11.94 min, 75.0 mg, crude) was purified by Prep-HPLC again with the following conditions (Column: CHIRAL ART Cellulose-SC, 2×25 cm, 5 m; Mobile Phase A: MtBE (0.5% 2M NH₃-MeOH)-HPLC, Mobile Phase B: MeOH:DCM=1:1-HPLC; Flow rate: 20 mL/min; Gradient: 30% B to 30% B in 8.5 min; Wave Length: 254/220 nm) to afford (1R)-2,2-difluoro-N-(3-{6-[1-hydroxypropyl]-4-methylpyridin-3-yl}-1-methyl-2-oxo-1,6-naphthyridin-7-yl)cyclopropane-1-carboxamide Isomer B (21.9 mg, 40%) as a white solid. RT2(min): 11.94; LCMS (ESI, m/z): [M+H]⁺=429.2. ¹H NMR (400 MHz, DMSO-d₆): δ 11.36 (s, 1H), 8.74 (s, 1H), 8.28 (s, 1H), 8.22 (s, 1H), 8.03 (s, 1H), 7.41 (s, 1H), 5.36-5.31 (m, 1H), 4.54-4.49 (m, 1H), 3.62 (s, 3H), 3.09-3.03 (m, 1H), 2.21 (s, 3H), 2.12-2.05 (m, 2H), 1.92-1.77 (m, 1H), 1.71-1.60 (m, 1H), 0.92-0.84 (m, 3H).

The absolute stereochemistry of Isomers A and B was not assigned. The two isomeric structures that could be obtained from chiral separation of the isomeric mixture as described above are shown as Compounds 125 and 126 in Table 1.

### Example S50. Synthesis of N-(3-(7-hydroxy-6,7-dihydro-5H-cyclopenta[b]pyridin-3-yl)-1-methyl-2-oxo-1,2-dihydro-1,6-naphthyridin-7-yl)cyclopropanecarboxamide (Compound 127)

### Step 1. Synthesis of 3-bromo-6,7-dihydro-5H-cyclopenta[b]pyridine 1-oxide

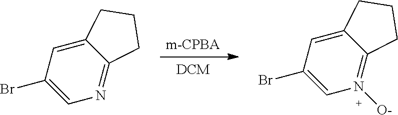

To a solution of 3-bromo-6,7-dihydro-5H-cyclopenta[b]pyridine (2.0 g, 10.10 mmol) in DCM (20.0 mL) was added m-CPBA (4.4 g, 25.25 mmol) at room temperature. The reaction mixture was stirred at room temperature for 1 h. After the reaction was completed, the resulting mixture was diluted with H₂O and extracted with dichloromethane. The combined organic layer was washed with NaHCO₃ solution, brine, dried over anhydrous sodium sulfate and filtered. The filtrate was concentrated under reduced pressure. The residue was purified by flash column chromatography with petroleum ether/ethyl acetate (40/60, v/v) to afford 3-bromo-6,7-dihydro-5H-cyclopenta[b]pyridine 1-oxide (1.9 g, 87%) as a light brown solid. LCMS (ESI, m/z): [M+H]⁺=214.0.

### Step 2. Synthesis of 3-bromo-6,7-dihydro-5H-cyclopenta[b]pyridin-7-yl acetate

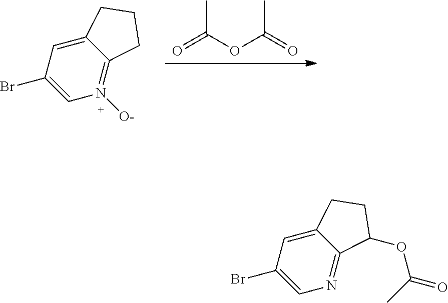

The solution of 3-bromo-6,7-dihydro-5H-cyclopenta[b]pyridine 1-oxide (1.9 g, 8.88 mmol) in acetic anhydride (20.0 mL) was stirred at 100° C. for 16 h. After the reaction was completed, The pH value of the mixture was adjusted to 7 with NaHCO₃ (aq.). The resulting mixture was diluted with H₂O and extracted with ethyl acetate. The combined organic layer was washed with brine, dried over anhydrous sodium sulfate and filtered. The filtrate was concentrated under reduced pressure. The residue was purified flash column chromatography with petroleum ether/ethyl acetate (50/50, v/v) to afford 3-bromo-6,7-dihydro-5H-cyclopenta[b]pyridin-7-yl acetate (1.1 g, 48%) as a red oil. LCMS (ESI, m/z): [M+H]⁺=256.0.

### Step 3. Synthesis of 3-(4,4,5,5-tetramethyl-1,3,2-dioxaborolan-2-yl)-6,7-dihydro-5H-cyclopenta[b]pyridin-7-yl acetate

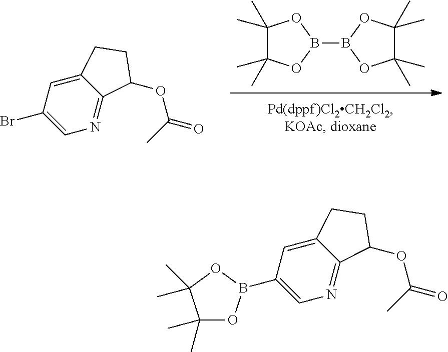

To a solution of 3-bromo-6,7-dihydro-5H-cyclopenta[b]pyridin-7-yl acetate (1.1 g, 6.44 mmol) in 1,4-dioxane (15.0 mL) was added 4,4,4′,4′,5,5,5′,5′-octamethyl-2,2′-bi(1,3,2-dioxaborolane) (1.6 g, 6.44 mmol), Pd(dppf)Cl₂·CH₂Cl₂ (351.1 mg, 0.64 mmol) and KOAc (1.3 g, 12.89 mmol) at room temperature under N₂. The resulting mixture was stirred at 80° C. for 4 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by reverse phase flash column chromatography with ACN/H₂O (20/80, v/v) to afford 3-(4,4,5,5-tetramethyl-1,3,2-dioxaborolan-2-yl)-6,7-dihydro-5H-cyclopenta[b]pyridin-7-yl acetate (560.0 mg, 43%) as a light brown solid. LCMS (ESI, m/z): [M+H]⁺=304.2.

### Step 4. Synthesis of 3-(7-chloro-1-methyl-2-oxo-1,2-dihydro-1,6-naphthyridin-3-yl)-6,7-dihydro-51H-cyclopenta[b]pyridin-7-yl acetate

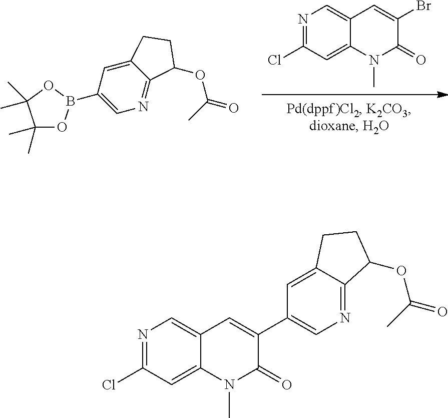 [SMILES: CC(=O)OC1CCc2cc(B3OC(C)(C)C(C)(C)O3)cnc21 ; Cn1c(=O)c(Br)cc2cnc(Cl)cc21 ; CC(=O)OC1CCc2cc(-c3cc4cnc(Cl)cc4n(C)c3=O)cnc21]

To a solution of 3-(4,4,5,5-tetramethyl-1,3,2-dioxaborolan-2-yl)-6,7-dihydro-5H-cyclopenta[b]pyridin-7-yl acetate (560.0 mg, 1.85 mmol) in 1,4-dioxane (8.0 mL) was added 3-bromo-7-chloro-1-methyl-1,6-naphthyridin-2(1H)-one (505.2 mg, 1.84 mmol), Pd(dppf)Cl₂ (135.2 mg, 0.19 mmol), K₂CO₃ (765.9 mg, 5.54 mmol) and H₂O (2.0 mL) at room temperature under N₂. The resulting mixture was stirred at 80° C. for 16 h under N₂. After the reaction was completed, the resulting mixture was filtered. The filtrate was concentrated under reduced pressure. The residue was purified by flash column chromatography with petroleum ether/ethyl acetate (40/60, v/v) to afford 3-(7-chloro-1-methyl-2-oxo-1,2-dihydro-1,6-naphthyridin-3-yl)-6,7-dihydro-5H-cyclopenta[b]pyridin-7-yl acetate (477.0 mg, 69%) as a yellow solid. LCMS (ESI, m/z): [M+H]⁺=370.1.

### Step 5. Synthesis of 3-(7-(cyclopropanecarboxamido)-1-methyl-2-oxo-1,2-dihydro-1,6-naphthyridin-3-yl)-6,7-dihydro-5H-cyclopenta[b]pyridin-7-yl acetate

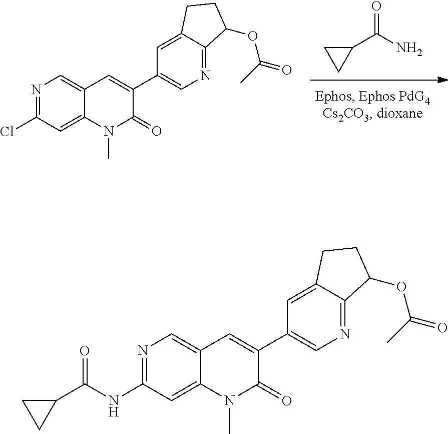 [SMILES: CC(=O)OC1CCc2cc(-c3cc4cnc(Cl)cc4n(C)c3=O)cnc21 ; NC(=O)C1CC1 ; CC(=O)OC1CCc2cc(-c3cc4cnc(NC(=O)C5CC5)cc4n(C)c3=O)cnc21]

To a solution of 3-(7-chloro-1-methyl-2-oxo-1,2-dihydro-1,6-naphthyridin-3-yl)-6,7-dihydro-5H-cyclopenta[b]pyridin-7-yl acetate (200.0 mg, 0.54 mmol) in 1,4-dioxane (10.0 mL) was added cyclopropanecarboxamide (230.1 mg, 2.71 mmol), EPhos (57.9 mg, 0.11 mmol), Cs₂CO₃ (528.6 mg, 1.62 mmol) and EPhos Pd G4 (49.7 mg, 0.05 mmol) at room temperature under N₂. The resulting mixture was stirred at 80° C. for 3 h under N₂. After the reaction was completed, the resulting mixture was concentrated under reduced pressure. The residue was purified by flash column chromatography with dichloromethane/methanol (96/4, v/v) to afford 3-(7-(cyclopropanecarboxamido)-1-methyl-2-oxo-1,2-dihydro-1,6-naphthyridin-3-yl)-6,7-dihydro-5H-cyclopenta[b]pyridin-7-yl acetate (110.0 mg, 48%) as a yellow solid. LCMS (ESI, m/z): [M+H]⁺=419.2.

### Step 6. Synthesis of N-(3-(7-hydroxy-6,7-dihydro-5H-cyclopenta[b]pyridin-3-yl)-1-methyl-2-oxo-1,2-dihydro-1,6-naphthyridin-7-yl)cyclopropanecarboxamide (Compound 127)

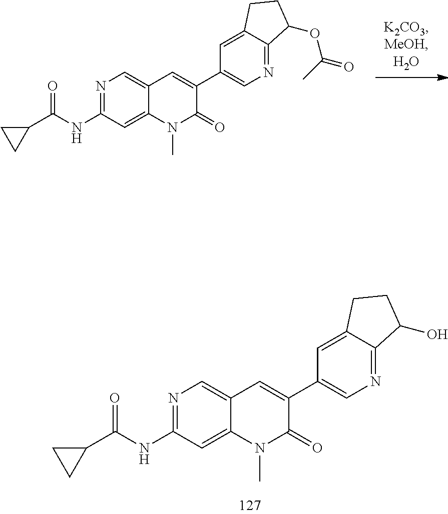 [SMILES: CC(=O)OC1CCc2cc(-c3cc4cnc(NC(=O)C5CC5)cc4n(C)c3=O)cnc21 ; Cn1c(=O)c(-c2cnc3c(c2)CCC3O)cc2cnc(NC(=O)C3CC3)cc21]

To a solution of 3-(7-(cyclopropanecarboxamido)-1-methyl-2-oxo-1,2-dihydro-1,6-naphthyridin-3-yl)-6,7-dihydro-5H-cyclopenta[b]pyridin-7-yl acetate (50.0 mg, 0.12 mmol) in MeOH (6.0 mL) was added K₂CO₃ (49.5 mg, 0.36 mmol) and H₂O (2.0 mL) at room temperature. The resulting mixture was stirred at room temperature for 3 h. After the reaction was completed, the mixture was filtered. The solid was washed with DMF and water and then collected to afford N-(3-(7-hydroxy-6,7-dihydro-5H-cyclopenta[b]pyridin-3-yl)-1-methyl-2-oxo-1,2-dihydro-1,6-naphthyridin-7-yl)cyclopropanecarboxamide (Compound 127) (7.0 mg, 15%) as an off-white solid. LCMS (ESI, m/z): [M+H]⁺=377.1. ¹H NMR (400 MHz, DMSO-d₆): δ 11.16 (s, 1H), 8.73-8.67 (m, 2H), 8.24-8.21 (m, 2H), 7.94 (s, 1H), 5.42 (d, J=5.6 Hz, 1H), 5.00-4.98 (m, 1H), 3.60 (s, 3H), 3.11-2.98 (m, 1H), 2.86-2.73 (m, 1H), 2.47-2.40 (m, 1H), 2.16-2.09 (m, 1H), 1.94-1.82 (m, 1H), 0.89-0.86 (m, 4H).

### Example S51. Synthesis of (1R,2R)-2-fluoro-N-(3-(6-((S)-1-hydroxybutyl-1-d)-4-methylpyridin-3-yl)-1-methyl-2-oxo-1,2-dihydro-1,6-naphthyridin-7-yl)cyclopropane-1-carboxamide and (1R,2R)-2-fluoro-N-(3-(6-((R)-1-hydroxybutyl-1-d)-4-methylpyridin-3-yl)-1-methyl-2-oxo-1,2-dihydro-1,6-naphthyridin-7-yl)cyclopropane-1-carboxamide (Compound 128 and 129)

### Step 1. Synthesis of (1R,2R)-2-fluoro-N-(3-(6-(1-hydroxybutyl-1-d)-4-methylpyridin-3-yl)-1-methyl-2-oxo-1,2-dihydro-1,6-naphthyridin-7-yl)cyclopropane-1-carboxamide

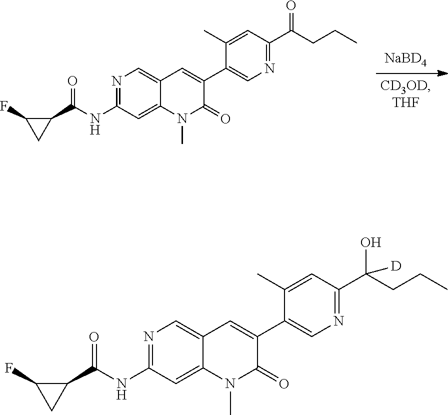

To a solution of (1R,2R)—N-[3-(6-butanoyl-4-methylpyridin-3-yl)-1-methyl-2-oxo-1,6-naphthyridin-7-yl]-2-fluorocyclopropane-1-carboxamide (400.0 mg, 0.94 mmol) in CD₃OD (2.0 mL) and THE (20.0 mL) was added NaBD₄ (179.1 mg, 4.74 mmol) at 0° C. The resulting mixture was stirred at 0° C. for 3 h. After the reaction was completed, the reaction mixture was quenched with water at 0° C. and then extracted with ethyl acetate. The combined organic layer was washed with brine, dried over anhydrous sodium sulfate and filtered. The filtrate was concentrated under reduced pressure. The residue was purified by flash column chromatography with dichloromethane/methanol (90/10, v/v) to afford (1R,2R)-2-fluoro-N-(3-(6-(1-hydroxybutyl-1-d)-4-methylpyridin-3-yl)-1-methyl-2-oxo-1,2-dihydro-1,6-naphthyridin-7-yl)cyclopropane-1-carboxamide (100.0 mg, 20%) as a yellow solid. LCMS (ESI, m/z): [M+H]⁺=426.2.

### Step 2. Chiral Separation of (1R,2R)-2-fluoro-N-(3-(6-((S)-1-hydroxybutyl-1-d)-4-methylpyridin-3-yl)-1-methyl-2-oxo-1,2-dihydro-1,6-naphthyridin-7-yl)cyclopropane-1-carboxamide and (1R,2R)-2-fluoro-N-(3-(6-((R)-1-hydroxybutyl-1-d)-4-methylpyridin-3-yl)-1-methyl-2-oxo-1,2-dihydro-1,6-naphthyridin-7-yl)cyclopropane-1-carboxamide (Compound 128 and 129)

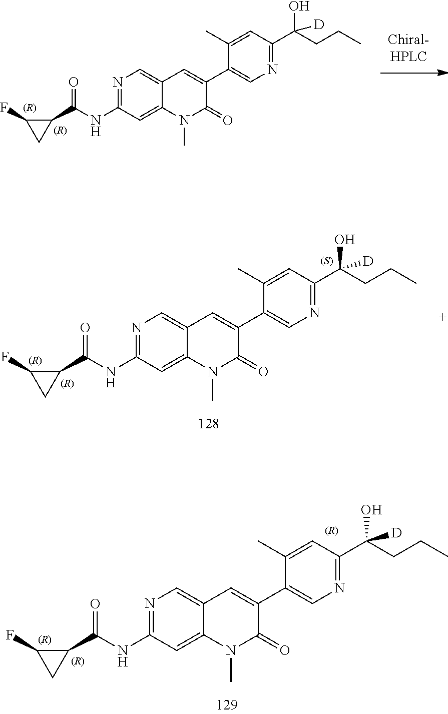

The racemic product of (1R,2R)-2-fluoro-N-(3-(6-(1-hydroxybutyl-1-d)-4-methylpyridin-3-yl)-1-methyl-2-oxo-1,2-dihydro-1,6-naphthyridin-7-yl)cyclopropane-1-carboxamide (100.0 mg, 0.24 mmol) was separated by Prep-Chiral-HPLC with the following conditions (Column: CHIRAL ART Amylose-SA, 2×25 cm, 5 m; Mobile Phase A: MtBE (0.5% 2M NH₃-MeOH)-HPLC, Mobile Phase B: EtOH:DCM=1:1-HPLC; Flow rate: 20 mL/min; Gradient: 20% B to 20% B in 19 min; Wave Length: 220/254 nm; RT1(min): 13.29; RT2(min): 17.08) to afford (1R,2R)-2-fluoro-N-(3-(6-((1-hydroxybutyl-1-d)-4-methylpyridin-3-yl)-1-methyl-2-oxo-1,2-dihydro-1,6-naphthyridin-7-yl)cyclopropane-1-carboxamide Isomer A (retention time 13.29 min, 33.3 mg, 66%) as a white solid and (1R,2R)-2-fluoro-N-(3-(6-(1-hydroxybutyl-1-d)-4-methylpyridin-3-yl)-1-methyl-2-oxo-1,2-dihydro-1,6-naphthyridin-7-yl)cyclopropane-1-carboxamide Isomer B (retention time 17.08 min, 35.8 mg, 70%) as a white solid.

Isomer A: retention time 1: 13.29 min, LCMS (ESI, m/z): [M+H]⁺=426.2. ¹H NMR (400 MHz, DMSO-d₆): δ 11.21 (s, 1H), 8.72 (s, 1H), 8.27-8.25 (m, 2H), 8.01 (s, 1H), 7.41 (s, 1H), 5.29 (s, 1H), 5.06-4.89 (m, 1H), 3.61 (s, 3H), 2.31-2.28 (m, 1H), 2.21 (s, 3H), 1.77-1.59 (m, 3H), 1.42-1.35 (m, 2H), 1.26-1.21 (m, 1H), 0.92-0.89 (m, 3H).

Isomer B: retention time 2: 17.08 min, LCMS (ESI, m/z): [M+H]⁺=426.2. ¹H NMR (400 MHz, DMSO-d₆): δ 11.21 (s, 1H), 8.72 (s, 1H), 8.27-8.25 (m, 2H), 8.01 (s, 1H), 7.41 (s, 1H), 5.29 (s, 1H), 5.06-4.90 (m, 1H), 3.61 (s, 3H), 2.30-2.23 (m, 1H), 2.21 (s, 3H), 1.77-1.59 (m, 3H), 1.39-1.32 (m, 2H), 1.25-1.20 (m, 1H), 0.92-0.89 (m, 3H).

The absolute stereochemistry of Isomers A and B was not assigned. The two isomeric structures that could be obtained from chiral separation of the isomeric mixture as described above are shown as Compounds 128 and 129 in Table 1.

### Example S52. Synthesis of (1S,2S)-2-fluoro-N-(3-(6-((S)-1-hydroxybutyl-1-d)-4-methylpyridin-3-yl)-1-methyl-2-oxo-1,2-dihydro-1,6-naphthyridin-7-yl)cyclopropane-1-carboxamide and (1S,2S)-2-fluoro-N-(3-(6-((R)-1-hydroxybutyl-1-d)-4-methylpyridin-3-yl)-1-methyl-2-oxo-1,2-dihydro-1,6-naphthyridin-7-yl)cyclopropane-1-carboxamide (Compound 130 and 131)

### Step 1. Synthesis of (1S,2S)-2-fluoro-N-(3-(6-(1-hydroxybutyl-1-d)-4-methylpyridin-3-yl)-1-methyl-2-oxo-1,2-dihydro-1,6-naphthyridin-7-yl)cyclopropane-1-carboxamide

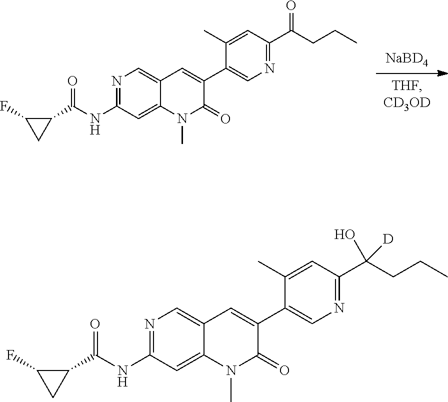

To a solution of (1S,2S)—N-[3-(6-butanoyl-4-methylpyridin-3-yl)-1-methyl-2-oxo-1,6-naphthyridin-7-yl]-2-fluorocyclopropane-1-carboxamide (250.0 mg, 0.59 mmol) in THF (8.0 mL) and CD₃OD (2.0 mL) was added NaBD₄ (111.9 mg, 2.96 mmol) at 0° C. under N₂. The resulting mixture was stirred at room temperature for 2 h. After the reaction was completed, the mixture was diluted with water and extracted with ethyl acetate. The combined organic layer was washed with brine, dried over anhydrous sodium sulfate and filtered. The filtrate was concentrated under reduced pressure. The residue was purified by flash column chromatography with dichloromethane/methanol (83/17, v/v) to afford (1S,2S)-2-fluoro-N-(3-(6-(1-hydroxybutyl-1-d)-4-methylpyridin-3-yl)-1-methyl-2-oxo-1,2-dihydro-1,6-naphthyridin-7-yl)cyclopropane-1-carboxamide (60.0 mg, 22%) as a brown solid. LCMS (ESI, m/z): [M+H]⁺=426.1.

### Step 2. Chiral Separation of (1S,2S)-2-fluoro-N-(3-(6-((S)-1-hydroxybutyl-1-d)-4-methylpyridin-3-yl)-1-methyl-2-oxo-1,2-dihydro-1,6-naphthyridin-7-yl)cyclopropane-1-carboxamide and (1S,2S)-2-fluoro-N-(3-(6-((R)-1-hydroxybutyl-1-d)-4-methylpyridin-3-yl)-1-methyl-2-oxo-1,2-dihydro-1,6-naphthyridin-7-yl)cyclopropane-1-carboxamide (Compound 130 and 131)

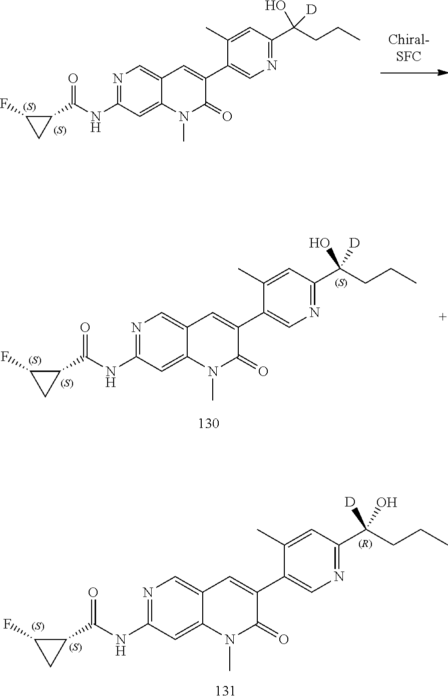

The product of (1S,2S)-2-fluoro-N-(3-(6-(1-hydroxybutyl-1-d)-4-methylpyridin-3-yl)-1-methyl-2-oxo-1,2-dihydro-1,6-naphthyridin-7-yl)cyclopropane-1-carboxamide (60.0 mg, 0.14 mmol) was separated by Prep-Chiral-HPLC with the following conditions: (Column: Lux 5 um Cellulose-4, 2.12×25 cm, 5 m; Mobile Phase A: Hex (0.5% 2M NH₃-MeOH)-HPLC, Mobile Phase B: MeOH:EtOH=1:1-HPLC; Flow rate: 20 mL/min; Gradient: 60% B to 60% B in 13.5 min; Wave Length: 254/220 nm; RT1(min): 9.70; RT2(min): 11.05) to afford (1S,2S)-2-fluoro-N-(3-(6-(1-hydroxybutyl-1-d)-4-methylpyridin-3-yl)-1-methyl-2-oxo-1,2-dihydro-1,6-naphthyridin-7-yl)cyclopropane-1-carboxamide Isomer A (retention time 9.70 min, 14.1 mg, 47%) as a white solid and (1S,2S)-2-fluoro-N-(3-(6-(1-hydroxybutyl-1-d)-4-methylpyridin-3-yl)-1-methyl-2-oxo-1,2-dihydro-1,6-naphthyridin-7-yl)cyclopropane-1-carboxamide Isomer B (retention time 11.05 min, 15.3 mg, 51%) as a white solid.

Isomer A: retention time 1: 9.70 min, LCMS (ESI, m/z): [M+H]⁺=426.1. ¹H NMR (400 MHz, DMSO-d₆): δ 11.19 (s, 1H), 8.72 (s, 1H), 8.27-8.25 (m, 2H), 8.01 (s, 1H), 7.40 (s, 1H), 5.27 (s, 1H), 5.08-4.89 (m, 1H), 3.68 (s, 3H), 2.37-2.27 (m, 1H), 2.21 (s, 3H), 1.78-1.59 (m, 3H), 1.42-1.31 (m, 2H), 1.24-1.21 (m, 1H), 0.93-0.85 (m, 3H).

Isomer B: retention time 2: 11.05 min, LCMS (ESI, m/z): [M+H]⁺=426.2. ¹H NMR (400 MHz, DMSO-d₆): δ 11.19 (s, 1H), 8.72 (s, 1H), 8.27-8.25 (m, 2H), 8.01 (s, 1H), 7.40 (s, 1H), 5.26 (s, 1H), 5.07-4.89 (m, 1H), 3.62 (s, 3H), 2.33-2.28 (m, 1H), 2.21 (s, 3H), 1.78-1.59 (m, 3H), 1.42-1.36 (m, 2H), 1.26-1.21 (m, 1H), 0.93-0.89 (m, 3H).

The absolute stereochemistry of isomers A and B was not assigned. The two isomeric structures that could be obtained from chiral separation of the isomeric mixture as described above are shown as Compounds 130 and 131 in Table 1.

### Example S53. Synthesis of (R)-2,2-difluoro-N-(3-(6-((S)-1-hydroxybutyl)-4-methylpyridin-3-yl)-1-methyl-2-oxo-1,2-dihydro-1,6-naphthyridin-7-yl) cyclopropane-1-carboxamide and (R)-2,2-difluoro-N-(3-(6-((R)-1-hydroxybutyl)-4-methylpyridin-3-yl)-1-methyl-2-oxo-1,2-dihydro-1,6-naphthyridin-7-yl) cyclopropane-1-carboxamide (Compound 132 and 133)

### Step 1. Synthesis of (R)—N-(3-(6-butyryl-4-methylpyridin-3-yl)-1-methyl-2-oxo-1,2-dihydro-1,6-naphthyridin-7-yl)-2,2-difluorocyclopropane-1-carboxamide

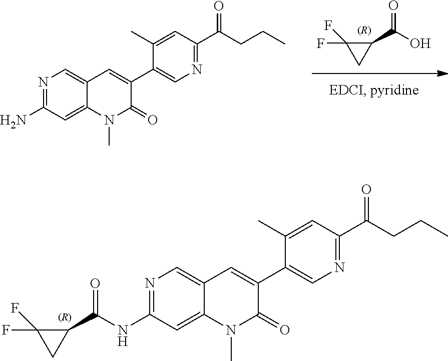

To a solution of 7-amino-3-(6-butanoyl-4-methylpyridin-3-yl)-1-methyl-1,6-naphthyridin-2-one (210.0 mg, 0.62 mmol) in pyridine (5.0 mL) was added (1R)-2,2-difluorocyclopropane-1-carboxylic acid (114.3 mg, 0.936 mmol) and EDCI (598.3 mg, 3.12 mmol) at room temperature. The resulting mixture was stirred at room temperature for 16 h. After the reaction was completed, the mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with dichloromethane/methanol (90/10, v/v) to afford (R)—N-(3-(6-butyryl-4-methylpyridin-3-yl)-1-methyl-2-oxo-1,2-dihydro-1,6-naphthyridin-7-yl)-2,2-difluorocyclopropane-1-carboxamide (197.0 mg, 71%) as a yellow solid. LCMS (ESI, m/z): [M+H]⁺=441.1.

### Step 2. Synthesis of (1R)-2,2-difluoro-N-(3-(6-(1-hydroxybutyl)-4-methylpyridin-3-yl)-1-methyl-2-oxo-1,2-dihydro-1,6-naphthyridin-7-yl) cyclopropane-1-carboxamide

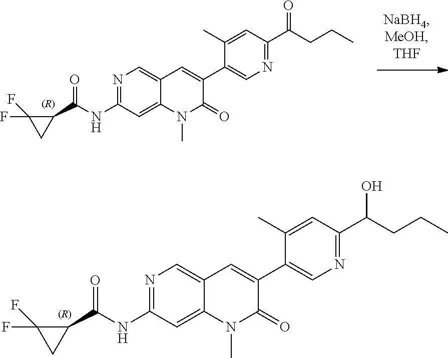

To a solution of (1R)—N-[3-(6-butanoyl-4-methylpyridin-3-yl)-1-methyl-2-oxo-1,6-naphthyridin-7-yl]-2,2-difluorocyclopropane-1-carboxamide (170.0 mg, 0.16 mmol) in THE (10.0 mL) and MeOH (2.0 mL) was added NaBH₄ (19.0 mg, 0.24 mmol) at room temperature. The resulting mixture was stirred at room temperature for 16 h. After the reaction was completed, the mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with dichloromethane/methanol (88/12, v/v) to afford (1R)-2,2-difluoro-N-(3-(6-(1-hydroxybutyl)-4-methylpyridin-3-yl)-1-methyl-2-oxo-1,2-dihydro-1,6-naphthyridin-7-yl) cyclopropane-1-carboxamide (60.0 mg, 30%) as a white solid. LCMS (ESI, m/z): [M+H]⁺=443.2.

### Step 3. Chiral Separation of (R)-2,2-difluoro-N-(3-(6-((S)-1-hydroxybutyl)-4-methylpyridin-3-yl)-1-methyl-2-oxo-1,2-dihydro-1,6-naphthyridin-7-yl) cyclopropane-1-carboxamide and (R)-2,2-difluoro-N-(3-(6-((R)-1-hydroxybutyl)-4-methylpyridin-3-yl)-1-methyl-2-oxo-1,2-dihydro-1,6-naphthyridin-7-yl) cyclopropane-1-carboxamide (Compound 132 and 133)

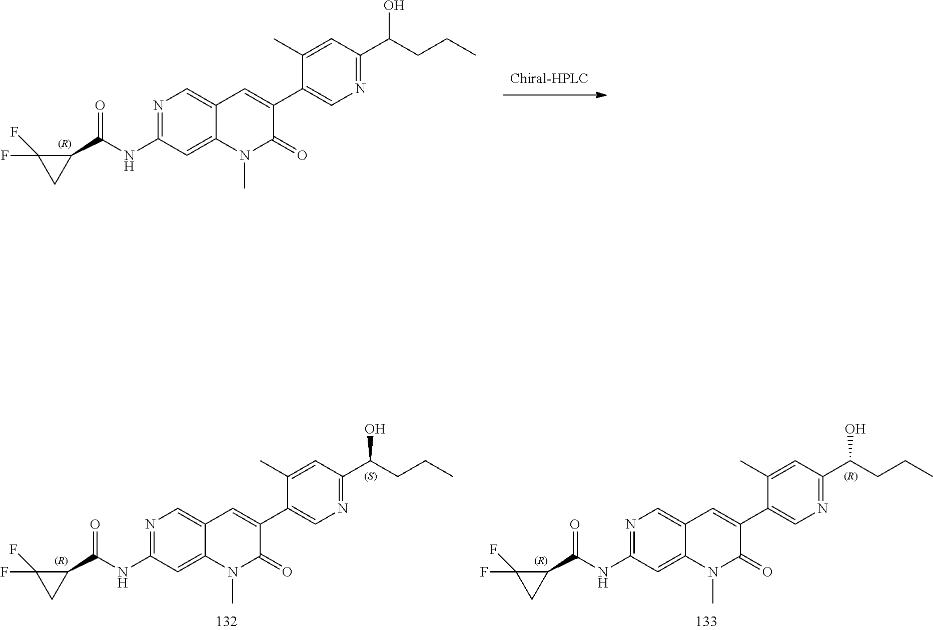

The racemic product of (1R)-2,2-difluoro-N-{3-[6-(1-hydroxybutyl)-4-methylpyridin-3-yl]-1-methyl-2-oxo-1,6-naphthyridin-7-yl}cyclopropane-1-carboxamide (60.0 mg, 0.14 mmol) was separated by Prep-Chiral-HPLC with the following conditions (Column: CHIRAL ART Cellulose-SC, 2×25 cm, 5 m; Mobile Phase A: Hex (0.1% TFA)-HPLC, Mobile Phase B: MeOH:DCM=1:1-HPLC; Flow rate: 20 mL/min; Gradient: 40% B to 40% B in 14 min; Wave Length: 254/220 nm; RT1(min): 9.16; RT2(min): 12.70) to afford (R)-2,2-difluoro-N-(3-(6-(1-hydroxybutyl)-4-methylpyridin-3-yl)-1-methyl-2-oxo-1,2-dihydro-1,6-naphthyridin-7-yl) cyclopropane-1-carboxamide Isomer A (retention time 9.16 min, 28.0 mg, 92%) and (R)-2,2-difluoro-N-(3-(6-(1-hydroxybutyl)-4-methylpyridin-3-yl)-1-methyl-2-oxo-1,2-dihydro-1,6-naphthyridin-7-yl) cyclopropane-1-carboxamide Isomer B (retention time 12.70, 19.0 mg, 64%).

Isomer A: retention time 1: 9.16 min, LCMS (ESI, m/z): [M⁺H]⁺=443.2. ¹H NMR (400 MHz, CD₃OD): δ 8.74 (s, 1H), 8.60 (s, 1H), 8.39 (s, 1H), 8.15 (s, 1H), 8.00 (s, 1H), 5.11-5.08 (m, 1H), 3.71 (s, 3H), 2.96-2.88 (m, 1H), 2.57 (s, 3H), 2.22-2.15 (m, 1H), 1.97-1.81 (m, 3H), 1.61-1.51 (m, 2H), 1.05-1.01 (m, 3H).

Isomer B: retention time 2: 12.70 min, LCMS (ESI, m/z): [M⁺H]⁺=443.2. ¹H NMR (400 MHz, CD₃OD): δ 8.74-8.71 (m, 1H), 8.60 (s, 1H), 8.39 (s, 1H), 8.18-8.15 (s, 1H), 8.01-7.93 (m, 1H), 5.11-5.08 (m, 1H), 3.78-3.74 (m, 3H), 2.96-2.91 (m, 1H), 2.57-2.52 (m, 3H), 2.22-2.15 (m, 1H), 1.97-1.82 (m, 3H), 1.61-1.51 (m, 2H), 1.05-1.02 (m, 3H).

The absolute stereochemistry of Isomers A and B was not assigned. The two isomeric structures that could be obtained from chiral separation of the isomeric mixture as described above are shown as Compounds 132 and 133 in Table 1.

### Example S54. Synthesis of (S)—N-(1-(2-hydroxy-2-methylpropyl)-3-(6-(1-hydroxybutyl)-4-methylpyridin-3-yl)-2-oxo-1,2-dihydro-1,6-naphthyridin-7-yl)cyclopropanecarboxamide and (R)—N-(1-(2-hydroxy-2-methylpropyl)-3-(6-(1-hydroxybutyl)-4-methylpyridin-3-yl)-2-oxo-1,2-dihydro-1,6-naphthyridin-7-yl)cyclopropanecarboxamide (Compound 134 and 135)

### Step 1. Synthesis of 3-bromo-7-chloro-1-(2-hydroxy-2-methylpropyl)-1,6-naphthyridin-2(1H)-one

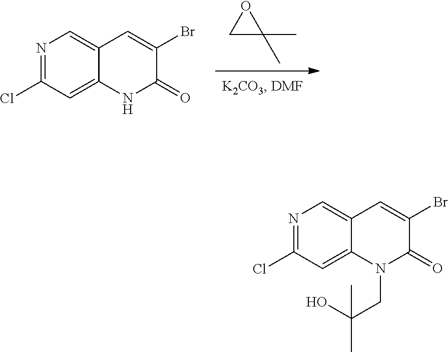

To a solution of 3-bromo-7-chloro-1,6-naphthyridin-2(1H)-one (500.0 mg, 1.93 mmol) in DMF (10.0 mL) was added 2,2-dimethyloxirane (305.7 mg, 4.24 mmol) and K₂CO₃ (532.6 mg, 3.85 mmol) at room temperature. The reaction mixture was stirred at 120° C. for 16 h. After the reaction was completed, the resulting mixture was cooled to room temperature and diluted with H₂O. The mixture was extracted with ethyl acetate. The combined organic layer was washed with brine, dried over anhydrous sodium sulfate and filtered. The filtrate was concentrated under reduced pressure. The residue was purified by flash column chromatography with CH₂Cl₂/MeOH (95/5, v/v) to afford 3-bromo-7-chloro-1-(2-hydroxy-2-methylpropyl)-1,6-naphthyridin-2(1H)-one (348.0 mg, 53%) as a white solid. LCMS (ESI, m/z): [M+H]⁺=331.2.

### Step 2. Synthesis of 3-bromo-1-(2-((tert-butyldimethylsilyl)oxy)-2-methylpropyl)-7-chloro-1,6-naphthyridin-2(1H)-one

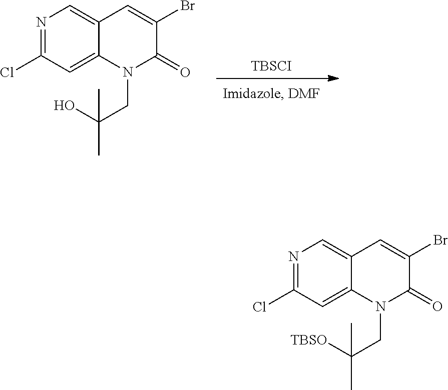 [SMILES: "TBSCI" ; CC(C)(O)Cn1c(=O)c(Br)cc2cnc(Cl)cc21 ; CC(C)(Cn1c(=O)c(Br)cc2cnc(Cl)cc21)O[Si](C)(C)C(C)(C)C]

To a solution of 3-bromo-7-chloro-1-(2-hydroxy-2-methylpropyl)-1,6-naphthyridin-2(1H)-one (308.0 mg, 0.93 mmol) in DMF (5.0 mL) was added Imidazole (189.7 mg, 2.72 mmol) and TBSCl (209.3 mg, 1.39 mmol) at 0° C. under N₂. The resulting mixture was stirred at room temperature for 16 h. After the reaction was completed, the resulting mixture was diluted with H₂O. The mixture was extracted with ethyl acetate. The combined organic layer was washed with brine, dried over anhydrous sodium sulfate and filtered. The filtrate was concentrated under reduced pressure to afford 3-bromo-1-(2-((tert-butyldimethylsilyl)oxy)-2-methylpropyl)-7-chloro-1,6-naphthyridin-2(1H)-one (226.0 mg, crude) as a white solid. LCMS (ESI, m/z): [M+H]⁺=445.1.

### Step 3. Synthesis of 1-(2-((tert-butyldimethylsilyl)oxy)-2-methylpropyl)-3-(6-butyryl-4-methylpyridin-3-yl)-7-chloro-1,6-naphthyridin-2(1H)-one

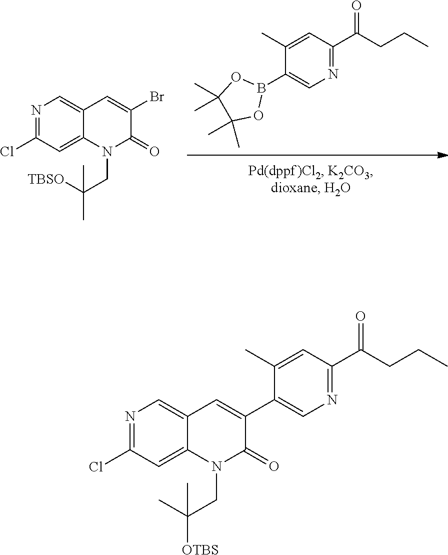 [SMILES: CC(C)(Cn1c(=O)c(Br)cc2cnc(Cl)cc21)O[Si](C)(C)C(C)(C)C ; CCCC(=O)c1cc(C)c(B2OC(C)(C)C(C)(C)O2)cn1 ; CCCC(=O)c1cc(C)c(-c2cc3cnc(Cl)cc3n(CC(C)(C)O[Si](C)(C)C(C)(C)C)c2=O)cn1]

To a solution of 3-bromo-1-(2-((tert-butyldimethylsilyl)oxy)-2-methylpropyl)-7-chloro-1,6-naphthyridin-2(1H)-one (300.0 mg, crude) in dioxane (15.0 mL) and H₂O (3.0 mL) was added 1-(4-methyl-5-(4,4,5,5-tetramethyl-1,3,2-dioxaborolan-2-yl)pyridin-2-yl)butan-1-one (239.3 mg, 0.828 mmol), Pd(dppf)Cl₂ (75.7 mg, 0.09 mmol) and K₂CO₃ (385.1 mg, 2.79 mmol) at room temperature under N₂. The resulting mixture was stirred at 80° C. for 16 h under N₂. After the reaction was completed, the resulting mixture was concentrated under reduced pressure. The residue was purified by flash column chromatography with CH₂Cl₂/EtOAc (40/60, v/v) to afford 1-(2-((tert-butyldimethylsilyl)oxy)-2-methylpropyl)-3-(6-butyryl-4-methylpyridin-3-yl)-7-chloro-1,6-naphthyridin-2(1H)-one (200.0 mg, 56%) as a white solid. LCMS (ESI, m/z): [M+H]⁺=528.1.

### Step 4. Synthesis of N-(1-(2-((tert-butyldimethylsilyl)oxy)-2-methylpropyl)-3-(6-butyryl-4-methylpyridin-3-yl)-2-oxo-1,2-dihydro-1,6-naphthyridin-7-yl)cyclopropanecarboxamide

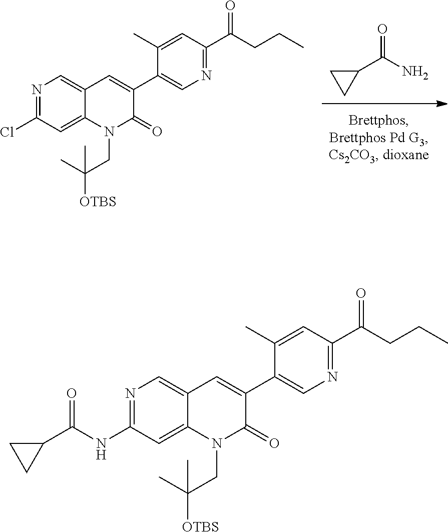

To a solution of 1-(2-((tert-butyldimethylsilyl)oxy)-2-methylpropyl)-3-(6-butyryl-4-methylpyridin-3-yl)-7-chloro-1,6-naphthyridin-2(1H)-one (171.0 mg, 0.33 mmol) in dioxane (6.0 mL) was added cyclopropanecarboxamide (70.3 mg, 0.83 mmol), BrettPhos (44.4 mg, 0.08 mmol), Cs₂CO₃ (403.8 mg, 1.24 mmol) and BrettPhos Pd G3 (37.5 mg, 0.04 mmol) at room temperature under N₂. The resulting mixture was stirred at 100° C. for 4 h under N₂. After the reaction was completed, the resulting mixture was filtered. The filtrate was concentrated under reduced pressure. The residue was purified by flash column chromatography with CH₂Cl₂/MeOH (92/8, v/v) to afford N-(1-(2-((tert-butyldimethylsilyl)oxy)-2-methylpropyl)-3-(6-butyryl-4-methylpyridin-3-yl)-2-oxo-1,2-dihydro-1,6-naphthyridin-7-yl)cyclopropanecarboxamide (100.0 mg, 53%) as a white solid. LCMS (ESI, m/z): [M+H]⁺=577.1.

### Step 5. Synthesis of N-(1-(2-((tert-butyldimethylsilyl)oxy)-2-methylpropyl)-3-(6-(1-hydroxybutyl)-4-methylpyridin-3-yl)-2-oxo-1,2-dihydro-1,6-naphthyridin-7-yl)cyclopropanecarboxamide

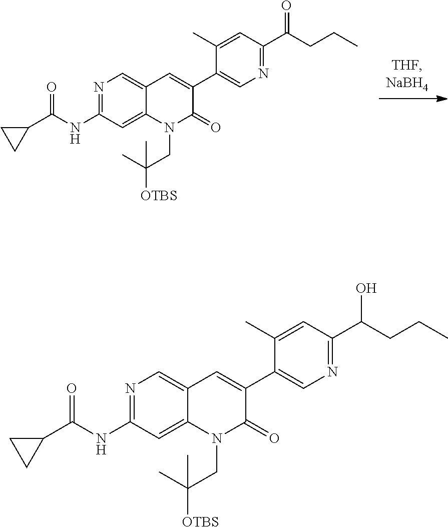 [SMILES: CCCC(=O)c1cc(C)c(-c2cc3cnc(NC(=O)C4CC4)cc3n(CC(C)(C)O[Si](C)(C)C(C)(C)C)c2=O)cn1 ; CCCC(O)c1cc(C)c(-c2cc3cnc(NC(=O)C4CC4)cc3n(CC(C)(C)O[Si](C)(C)C(C)(C)C)c2=O)cn1]

To a solution of N-(1-(2-((tert-butyldimethylsilyl)oxy)-2-methylpropyl)-3-(6-butyryl-4-methylpyridin-3-yl)-2-oxo-1,2-dihydro-1,6-naphthyridin-7-yl)cyclopropanecarboxamide (100.0 mg, 0.17 mmol) in THF (8.0 mL) was added NaBH₄ (12.3 mg, 0.32 mmol) at room temperature. The resulting mixture was stirred at room temperature for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/MeOH (88/12, v/v) to afford N-(1-(2-((tert-butyldimethylsilyl)oxy)-2-methylpropyl)-3-(6-(1-hydroxybutyl)-4-methylpyridin-3-yl)-2-oxo-1,2-dihydro-1,6-naphthyridin-7-yl)cyclopropanecarboxamide (60.7 mg, 61%) as a white solid. LCMS (ESI, m/z): [M+H]⁺=579.2.

### Step 6. Synthesis of N-(1-(2-hydroxy-2-methylpropyl)-3-(6-(1-hydroxybutyl)-4-methylpyridin-3-yl)-2-oxo-1,2-dihydro-1,6-naphthyridin-7-yl)cyclopropanecarboxamide

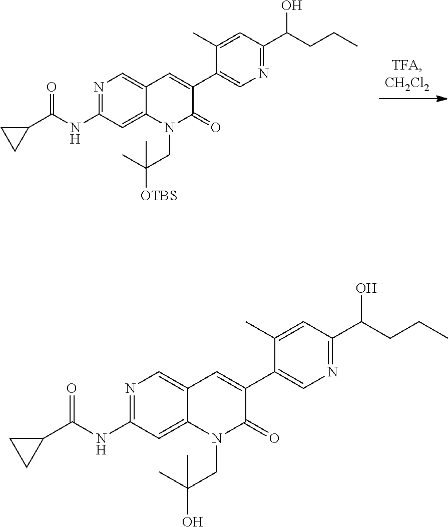 [SMILES: CCCC(O)c1cc(C)c(-c2cc3cnc(NC(=O)C4CC4)cc3n(CC(C)(C)O[Si](C)(C)C(C)(C)C)c2=O)cn1 ; CCCC(O)c1cc(C)c(-c2cc3cnc(NC(=O)C4CC4)cc3n(CC(C)(C)O)c2=O)cn1]

To a solution of N-(1-(2-((tert-butyldimethylsilyl)oxy)-2-methylpropyl)-3-(6-(1-hydroxybutyl)-4-methylpyridin-3-yl)-2-oxo-1,2-dihydro-1,6-naphthyridin-7-yl)cyclopropanecarboxamide (100.0 mg, 0.17 mmol) in CH₂Cl₂ (4.0 mL) was added TFA (1.0 mL) at room temperature. The resulting mixture was stirred at room temperature for 2 h. After the reaction was completed, the resulting mixture was diluted with H₂O and extracted with CH₂Cl₂. The combined organic layer was washed with brine, dried over anhydrous sodium sulfate and filtered. The filtrate was concentrated under reduced pressure. The residue was purified by flash column chromatography with CH₂Cl₂/MeOH (85/15, v/v) to afford N-(1-(2-hydroxy-2-methylpropyl)-3-(6-(1-hydroxybutyl)-4-methylpyridin-3-yl)-2-oxo-1,2-dihydro-1,6-naphthyridin-7-yl)cyclopropanecarboxamide (82.0 mg, 90%) as a white solid. LCMS (ESI, m/z): [M+H]⁺=465.2.

### Step 7. Chiral Separation of (S)—N-(1-(2-hydroxy-2-methylpropyl)-3-(6-(1-hydroxybutyl)-4-methylpyridin-3-yl)-2-oxo-1,2-dihydro-1,6-naphthyridin-7-yl)cyclopropanecarboxamide and (R)—N-(1-(2-hydroxy-2-methylpropyl)-3-(6-(1-hydroxybutyl)-4-methylpyridin-3-yl)-2-oxo-1,2-dihydro-1,6-naphthyridin-7-yl)cyclopropanecarboxamide (Compound 134 and 135)

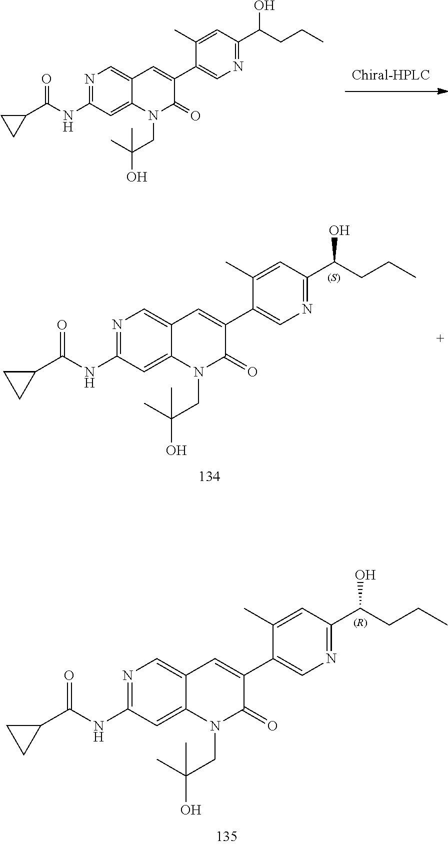

The racemic product of N-(1-(2-hydroxy-2-methylpropyl)-3-(6-(1-hydroxybutyl)-4-methylpyridin-3-yl)-2-oxo-1,2-dihydro-1,6-naphthyridin-7-yl)cyclopropanecarboxamide (82.0 mg, 0.18 mmol) was separated by Prep-Chiral-HPLC with the following conditions (Column: Lux 5 um Cellulose-4, 2.12×25 cm, 5 m; Mobile Phase A: Hex (0.5% 2M NH₃-MeOH)-HPLC, Mobile Phase B: MeOH:EtOH=1:1-HPLC; Flow rate: 20 mL/min; Gradient: 90% B to 90% B in 21 min; Wave Length: 254/220 nm; RT1(min): 9.46; RT2(min): 17.18) to afford N-(1-(2-hydroxy-2-methylpropyl)-3-(6-(1-hydroxybutyl)-4-methylpyridin-3-yl)-2-oxo-1,2-dihydro-1,6-naphthyridin-7-yl)cyclopropanecarboxamide Enantiomer A (retention time 9.46 min, 26.4 mg, crude) as a white solid and N-(1-(2-hydroxy-2-methylpropyl)-3-(6-(1-hydroxybutyl)-4-methylpyridin-3-yl)-2-oxo-1,2-dihydro-1,6-naphthyridin-7-yl)cyclopropanecarboxamide Enantiomer B (retention time 17.18 min, 28.2 mg, crude) as a white solid.

The crude product of N-(1-(2-hydroxy-2-methylpropyl)-3-(6-(1-hydroxybutyl)-4-methylpyridin-3-yl)-2-oxo-1,2-dihydro-1,6-naphthyridin-7-yl)cyclopropanecarboxamide Enantiomer A (retention time 9.46 min, 26.4 mg) was purified by Prep-HPLC with the following conditions (Column: XSelect CSH Prep C18 OBD Column, 19×250 mm, 5 m; Mobile Phase A: Water (0.05% FA), Mobile Phase B: ACN; Flow rate: 25 mL/min; Gradient: 24% B to 29% B in 8 min; Wave Length: 220 nm) to afford N-(1-(2-hydroxy-2-methylpropyl)-3-(6-(1-hydroxybutyl)-4-methylpyridin-3-yl)-2-oxo-1,2-dihydro-1,6-naphthyridin-7-yl)cyclopropanecarboxamide Enantiomer A (retention time 9.46 min, 9.3 mg, 22%) as a white solid.

The crude product of N-(1-(2-hydroxy-2-methylpropyl)-3-(6-(1-hydroxybutyl)-4-methylpyridin-3-yl)-2-oxo-1,2-dihydro-1,6-naphthyridin-7-yl)cyclopropanecarboxamide Enantiomer B (retention time 17.18 min, 28.2 mg) was purified by Prep-HPLC with the following conditions (Column: XBridge Prep OBD C18 Column, 30×150 mm, 5 m; Mobile Phase A: Water (10 mmol/L NH₄HCO₃), Mobile Phase B: ACN; Flow rate: 60 mL/min; Gradient: 29% B to 37% B in 8 min; Wave Length: 254 nm) to afford N-(1-(2-hydroxy-2-methylpropyl)-3-(6-(1-hydroxybutyl)-4-methylpyridin-3-yl)-2-oxo-1,2-dihydro-1,6-naphthyridin-7-yl)cyclopropanecarboxamide Enantiomer B (retention time 17.18 min, 8.8 mg, 21%) as a white solid.

Enantiomer A: retention time 1: 9.46 min, LCMS (ESI, m/z): [M+H]⁺=465.3. ¹H NMR (400 MHz, DMSO-d₆): δ 11.00 (s, 1H), 8.67 (s, 1H), 8.56 (s, 1H), 8.26 (s, 1H), 8.01 (s, 1H), 7.39 (s, 1H), 5.30 (d, J=4.8 Hz, 1H), 4.65 (s, 1H), 4.59-4.57 (m, 1H), 4.23 (s, 2H), 2.20 (s, 3H), 2.09-2.05 (m, 1H), 1.75-1.70 (m, 1H), 1.65-1.61 (m, 1H), 1.42-1.35 (m, 2H), 1.19 (s, 6H), 0.93-0.85 (m, 7H).

Enantiomer B: retention time 2: 17.18 min, LCMS (ESI, m/z): [M+H]⁺=465.3. ¹H NMR (400 MHz, DMSO-d₆): δ 10.99 (s, 1H), 8.67 (s, 1H), 8.56 (s, 1H), 8.26 (s, 1H), 8.01 (s, 1H), 7.39 (s, 1H), 5.28 (d, J=5.2 Hz, 1H), 4.64 (s, 1H), 4.61-4.56 (m, 1H), 4.23 (s, 2H), 2.20 (s, 3H), 2.09-2.05 (m, 1H), 1.75-1.72 (m, 1H), 1.66-1.61 (m, 1H), 1.41-1.36 (m, 2H), 1.18 (s, 6H), 0.92-0.84 (m, 7H).

The absolute stereochemistry of enantiomers A and B was not assigned. The two enantiomeric structures that could be obtained from chiral separation of the enantiomeric mixture as described above are shown as Compounds 134 and 135 in Table 1.

### Example S55. Synthesis of (S)—N-(3-(6-(1-hydroxybutyl)-4-methylpyridin-3-yl)-1-(2-hydroxyethyl)-2-oxo-1,2-dihydro-1,6-naphthyridin-7-yl)cyclopropanecarboxamide and (R)—N-(3-(6-(1-hydroxybutyl)-4-methylpyridin-3-yl)-1-(2-hydroxyethyl)-2-oxo-1,2-dihydro-1,6-naphthyridin-7-yl)cyclopropanecarboxamide (Compound 136 and 137)

### Step 1. Synthesis of 3-bromo-1-{2-[(tert-butyldimethylsilyl)oxy]ethyl}-7-chloro-1,6-naphthyridin-2-one

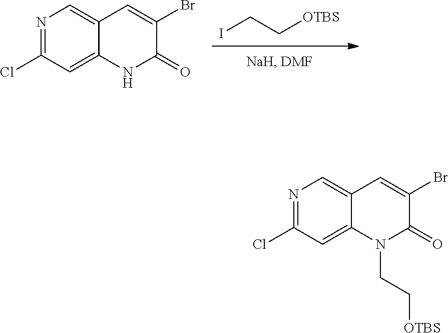

To a stirred mixture of 3-bromo-7-chloro-1H-1,6-naphthyridin-2-one (3.0 g, 11.56 mmol) in DMF (35.0 mL) was added NaH (0.6 g, 60%) at 0° C. under N₂. The resulting mixture was stirred at 0° C. for 0.5 h under N₂. Then tert-butyl(2-iodoethoxy)dimethylsilane (16.6 g, 57.81 mmol) was added to the mixture at 0° C. The resulting mixture was stirred at 120° C. for additional 2 h. After the reaction was completed, the resulting mixture was cooled to room temperature and then diluted with H₂O at 0° C. The resulting mixture was extracted with ethyl acetate. The combined organic layer was washed with brine, dried over anhydrous sodium sulfate and filtered. The filtrate was concentrated under reduced pressure. The residue was purified by flash column chromatography with ethyl acetate/petroleum ether (1/1, v/v) to afford 3-bromo-1-{2-[(tert-butyldimethylsilyl)oxy]ethyl}-7-chloro-1,6-naphthyridin-2-one (1.3 g, 26%) as a white solid. LCMS (ESI, m/z): [M+H]⁺=417.0.

### Step 2. Synthesis of 3-(6-butanoyl-4-methylpyridin-3-yl)-1-{2-[(tert-butyldimethylsilyl)oxy]ethyl}-7-chloro-1,6-naphthyridin-2-one

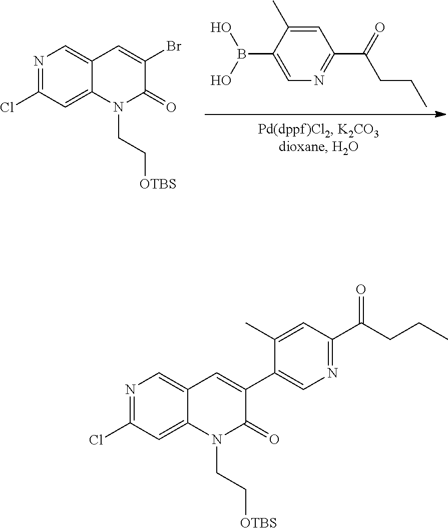 [SMILES: CC(C)(C)[Si](C)(C)OCCn1c(=O)c(Br)cc2cnc(Cl)cc21 ; CCCC(=O)c1cc(C)c(B(O)O)cn1 ; CCCC(=O)c1cc(C)c(-c2cc3cnc(Cl)cc3n(CCO[Si](C)(C)C(C)(C)C)c2=O)cn1]

To a solution of 3-bromo-1-{2-[(tert-butyldimethylsilyl)oxy]ethyl}-7-chloro-1,6-naphthyridin-2-one (1.3 g, 3.11 mmol) in dioxane (12.0 mL) and H₂O (3.0 mL) was added 6-butanoyl-4-methylpyridin-3-ylboronic acid (966.3 mg, 4.67 mmol), Pd(dppf)Cl₂ (455.4 mg, 0.62 mmol) and K₂CO₃ (1.3 g, 9.34 mmol) at room temperature under N₂. The resulting mixture was stirred at 80° C. for 16 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with ethyl acetate/petroleum ether (1/1, v/v) to afford 3-(6-butanoyl-4-methylpyridin-3-yl)-1-{2-[(tert-butyldimethylsilyl)oxy]ethyl}-7-chloro-1,6-naphthyridin-2-one (740.0 mg, 47%) as a yellow solid. LCMS (ESI, m/z): [M+H]⁺=500.2.

### Step 3. Synthesis of N-[3-(6-butanoyl-4-methylpyridin-3-yl)-1-{2-[(tert-butyldimethylsilyl)oxy]ethyl}-2-oxo-1,6-naphthyridin-7-yl]cyclopropanecarboxamide

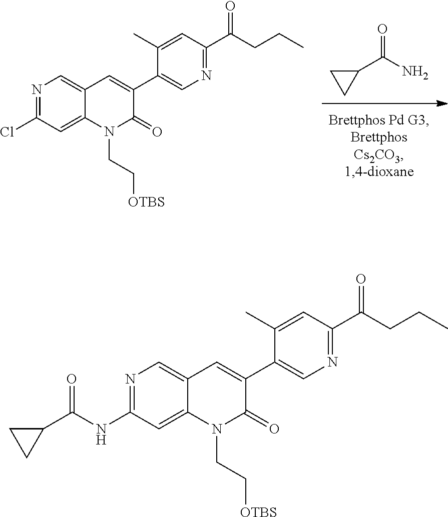

To a mixture of 3-(6-butanoyl-4-methylpyridin-3-yl)-1-{2-[(tert-butyldimethylsilyl)oxy]ethyl}-7-chloro-1,6-naphthyridin-2-one (740 mg, 1.48 mmol) in 1,4-dioxane (10.0 mL) was added cyclopropanecarboxamide (188.9 mg, 2.22 mmol), Brettphos Pd G3 (268.3 mg, 0.30 mmol), BrettPhos (317.7 mg, 0.59 mmol) and Cs₂CO₃ (1.5 g, 4.44 mmol) at room temperature under N₂. The resulting mixture was stirred at 85° C. for 16 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with ethyl acetate/petroleum ether (1/1, v/v) to afford N-[3-(6-butanoyl-4-methylpyridin-3-yl)-1-{2-[(tert-butyldimethylsilyl)oxy]ethyl}-2-oxo-1,6-naphthyridin-7-yl]cyclopropanecarboxamide (650.0 mg, 80%) as a yellow solid. LCMS (ESI, m/z): [M+H]⁺=549.3.

### Step 4. Synthesis of N-[3-(6-butanoyl-4-methylpyridin-3-yl)-1-(2-hydroxyethyl)-2-oxo-1,6-naphthyridin-7-yl]cyclopropanecarboxamide

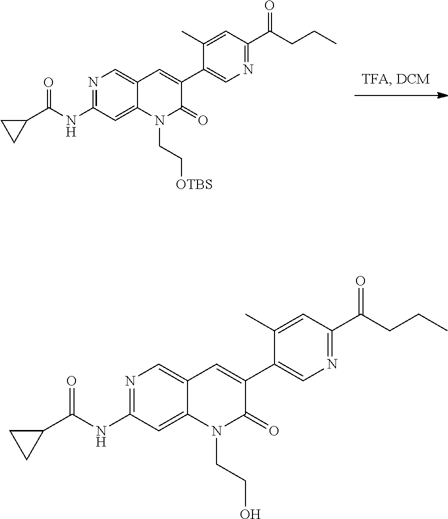 [SMILES: CCCC(=O)c1cc(C)c(-c2cc3cnc(NC(=O)C4CC4)cc3n(CCO[Si](C)(C)C(C)(C)C)c2=O)cn1 ; CCCC(=O)c1cc(C)c(-c2cc3cnc(NC(=O)C4CC4)cc3n(CCO)c2=O)cn1]

To a mixture of N-[3-(6-butanoyl-4-methylpyridin-3-yl)-1-{2-[(tert-butyldimethylsilyl)oxy]ethyl}-2-oxo-1,6-naphthyridin-7-yl]cyclopropanecarboxamide (600.0 mg, 1.09 mmol) in DCM (8.0 mL) was added TFA (8.0 mL) at room temperature. The mixture was stirred at room temperature for 1 h. After the reaction was completed, the resulting mixture was neutralized to pH=7 with saturated NaHCO₃ (aq). The resulting mixture was extracted with DCM. The combined organic layers were washed with brine, dried over anhydrous sodium sulfate and filtered. The filtrate was concentrated under reduced pressure. The residue was purified by flash column chromatography with ethyl acetate/petroleum ether (1/1, v/v) to afford N-[3-(6-butanoyl-4-methylpyridin-3-yl)-1-(2-hydroxyethyl)-2-oxo-1,6-naphthyridin-7-yl]cyclopropanecarboxamide (339.0 mg, 710%) as a white solid. LCMS (ESI, m/z): [M+H]⁺=435.2.

### Step 5. Synthesis of N-{3-[6-(1-hydroxybutyl)-4-methylpyridin-3-yl]-1-(2-hydroxyethyl)-2-oxo-1,6-naphthyridin-7-yl}cyclopropanecarboxamide

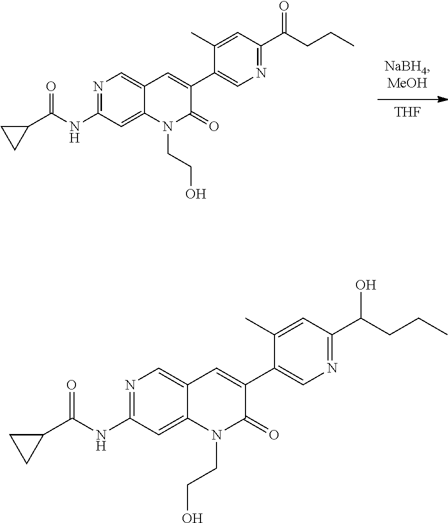 [SMILES: CCCC(=O)c1cc(C)c(-c2cc3cnc(NC(=O)C4CC4)cc3n(CCO)c2=O)cn1 ; CCCC(O)c1cc(C)c(-c2cc3cnc(NC(=O)C4CC4)cc3n(CCO)c2=O)cn1 ; "THF"]

To a mixture of N-[3-(6-butanoyl-4-methylpyridin-3-yl)-1-(2-hydroxyethyl)-2-oxo-1,6-naphthyridin-7-yl]cyclopropanecarboxamide (315.0 mg, 0.73 mmol) in MeOH (2.0 mL) and THF (8.0 mL) was added NaBH₄ (137.1 mg, 3.63 mmol) at 0° C. The resulting mixture was stirred at 0° C. for 2 h. After the reaction was completed, the reaction mixture was quenched with H₂O at 0° C. The resulting mixture was extracted with ethyl acetate. The combined organic layer was washed with brine, dried over anhydrous sodium sulfate and filtered. The filtrate was concentrated under reduced pressure. The residue was purified by flash column chromatography with CH₂Cl₂/MeOH (9/1, v/v) to afford N-{3-[6-(1-hydroxybutyl)-4-methylpyridin-3-yl]-1-(2-hydroxyethyl)-2-oxo-1,6-naphthyridin-7-yl}cyclopropanecarboxamide (229.0 mg, 72%) as a white solid. LCMS (ESI, m/z): [M+H]⁺=437.2.

### Step 6. Chiral Separation of (S)—N-(3-(6-(1-hydroxybutyl)-4-methylpyridin-3-yl)-1-(2-hydroxyethyl)-2-oxo-1,2-dihydro-1,6-naphthyridin-7-yl)cyclopropanecarboxamide and (R)—N-(3-(6-(1-hydroxybutyl)-4-methylpyridin-3-yl)-1-(2-hydroxyethyl)-2-oxo-1,2-dihydro-1,6-naphthyridin-7-yl)cyclopropanecarboxamide (Compound 136 and 137)

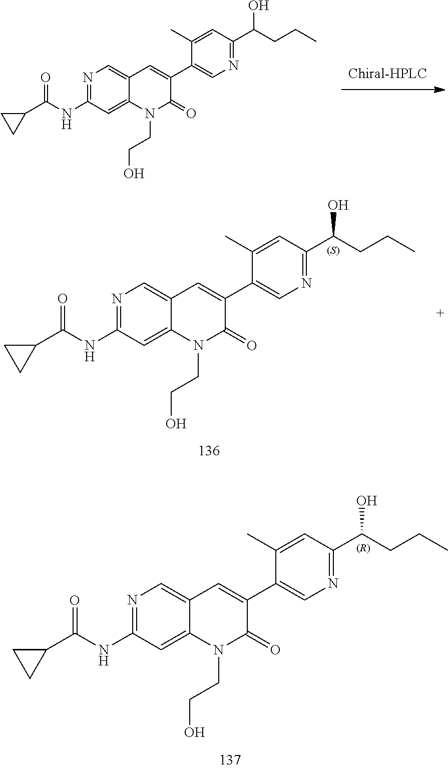

The product of N-{3-[6-(1-hydroxybutyl)-4-methylpyridin-3-yl]-1-(2-hydroxyethyl)-2-oxo-1,6-naphthyridin-7-yl}cyclopropanecarboxamide (100.0 mg, 0.23 mmol) was separated by Prep-Chiral-HPLC with the following conditions (Column: CHIRAL ART Cellulose-SB, 2×25 cm, 5 m; Mobile Phase A: Hex(0.5% 2M NH₃-MeOH)-HPLC, Mobile Phase B: MeOH:EtOH=1:1-HPLC; Flow rate: 20 mL/min; Gradient: 60% B to 60% B in 17 min; Wave Length: 220/254 nm; RT1(min): 10.76; RT2(min): 14.15) to afford N-(3-(6-(1-hydroxybutyl)-4-methylpyridin-3-yl)-1-(2-hydroxyethyl)-2-oxo-1,2-dihydro-1,6-naphthyridin-7-yl)cyclopropanecarboxamide Enantiomer A (retention time 10.76, 37.1 mg, 74%) as a white solid and N-(3-(6-(1-hydroxybutyl)-4-methylpyridin-3-yl)-1-(2-hydroxyethyl)-2-oxo-1,2-dihydro-1,6-naphthyridin-7-yl)cyclopropanecarboxamide Enantiomer B (retention time 14.15 min, 31.1 mg, 62%) as a white solid.

Enantiomer A: retention time 1: 10.76 min, LCMS (ESI, m/z): [M+H]⁺=437.3. ¹H NMR (400 MHz, DMSO-d₆): δ 11.10 (s, 1H), 8.70 (s, 1H), 8.37 (s, 1H), 8.27 (s, 1H), 8.01 (s, 1H), 7.40 (s, 1H), 5.30 (d, J=4.8 Hz, 1H), 4.99-4.97 (m, 1H), 4.59-4.57 (m, 1H), 4.27-4.24 (m, 2H), 3.70-3.66 (m, 2H), 2.20 (s, 3H), 2.10-2.05 (m, 1H), 1.79-1.70 (m, 1H), 1.67-1.58 (m, 1H), 1.44-1.34 (m, 2H), 0.92-0.84 (m, 7H).

Enantiomer B: retention time 2: 14.15 min, LCMS (ESI, m/z): [M+H]⁺=437.3. ¹H NMR (400 MHz, DMSO-d₆): δ 11.10 (s, 1H), 8.70 (s, 1H), 8.37 (s, 1H), 8.28 (s, 1H), 8.01 (s, 1H), 7.40 (s, 1H), 5.30 (s, 1H), 5.02-4.97 (m, 1H), 4.62-4.57 (m, 1H), 4.28-4.24 (m, 2H), 3.70-3.66 (m, 2H), 2.21 (s, 3H), 2.10-2.05 (m, 1H), 1.79-1.70 (m, 1H), 1.67-1.58 (m, 1H), 1.44-1.34 (m, 2H), 0.92-0.84 (m, 7H).

The absolute stereochemistry of enantiomers A and B was not assigned. The two enantiomeric structures that could be obtained from chiral separation of the enantiomeric mixture as described above are shown as Compounds 136 and 137 in Table 1.

### Example S56. Synthesis of (S)—N-(1-(2-fluoroethyl)-3-(6-(1-hydroxybutyl)-4-methylpyridin-3-yl)-2-oxo-1,2-dihydro-1,6-naphthyridin-7-yl)cyclopropanecarboxamide and (R)—N-(1-(2-fluoroethyl)-3-(6-(1-hydroxybutyl)-4-methylpyridin-3-yl)-2-oxo-1,2-dihydro-1,6-naphthyridin-7-yl)cyclopropanecarboxamide (Compound 138 and 139)

### Step 1. Synthesis of 3-bromo-7-chloro-1-(2-fluoroethyl)-1,6-naphthyridin-2(1H)-one

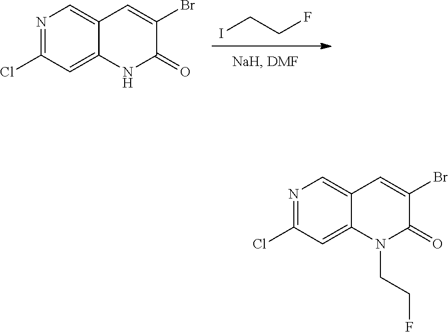

To a solution of 3-bromo-7-chloro-1,6-naphthyridin-2(1H)-one (1.5 g, 5.78 mmol) in DMF (15.0 mL) was added NaH (0.5 g, 60%) at 0° C. under N₂. The resulting mixture was stirred at 0° C. for 30 min. Then 1-fluoro-2-iodoethane (5.0 g, 28.91 mmol) was added to the mixture at room temperature. The resulting mixture was stirred at room temperature for additional 30 min and then stirred at 80° C. for 2 h. After the reaction was completed, the reaction was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (53/47, v/v) to afford 3-bromo-7-chloro-1-(2-fluoroethyl)-1,6-naphthyridin-2(1H)-one (1.0 g, 56%) as a yellow solid. LCMS (ESI, m/z): [M+H]⁺=304.9.

### Step 2. Synthesis of 3-(6-butyryl-4-methylpyridin-3-yl)-7-chloro-1-(2-fluoroethyl)-1,6-naphthyridin-2(1H)-one

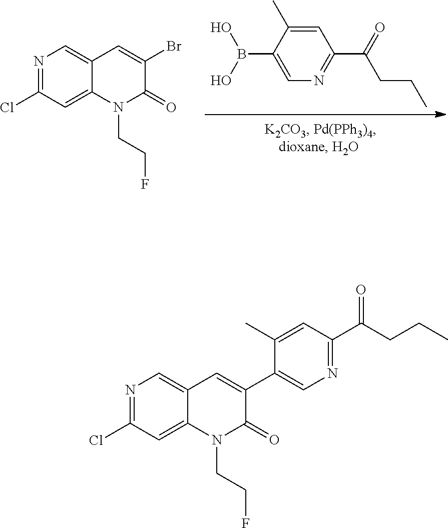 [SMILES: O=c1c(Br)cc2cnc(Cl)cc2n1CCF ; CCCC(=O)c1cc(C)c(B(O)O)cn1 ; CCCC(=O)c1cc(C)c(-c2cc3cnc(Cl)cc3n(CCF)c2=O)cn1]

To a solution of 3-bromo-7-chloro-1-(2-fluoroethyl)-1,6-naphthyridin-2(1H)-one (600.0 mg, 1.96 mmol) in dioxane/H₂O (10.0 mL/2.0 mL) was added (6-butyryl-4-methylpyridin-3-yl)boronic acid (406.6 mg, 1.96 mmol), K₂CO₃ (814.2 mg, 5.89 mmol) and Pd(PPh₃)₄ (226.9 mg, 0.19 mmol) at room temperature under N₂. The resulting mixture was stirred at 80° C. for 16 h. After the reaction was completed, the mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (60/40, v/v) to afford 3-(6-butyryl-4-methylpyridin-3-yl)-7-chloro-1-(2-fluoroethyl)-1,6-naphthyridin-2(1H)-one (530.0 mg, 69%) as a white solid. LCMS (ESI, m/z): [M+H]⁺=388.1.

### Step 3. Synthesis of N-(3-(6-butyryl-4-methylpyridin-3-yl)-1-(2-fluoroethyl)-2-oxo-1,2-dihydro-1,6-naphthyridin-7-yl)cyclopropanecarboxamide

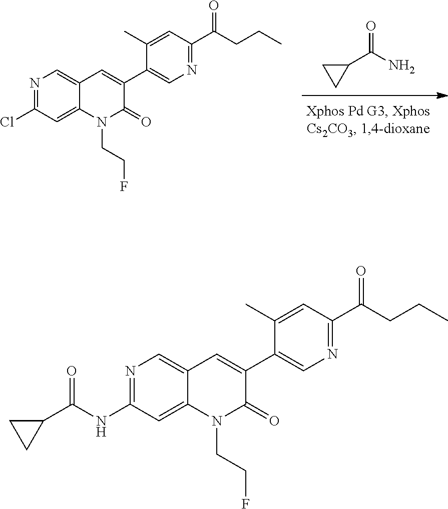 [SMILES: CCCC(=O)c1cc(C)c(-c2cc3cnc(Cl)cc3n(CCF)c2=O)cn1 ; NC(=O)C1CC1 ; CCCC(=O)c1cc(C)c(-c2cc3cnc(NC(=O)C4CC4)cc3n(CCF)c2=O)cn1]

To a solution of 3-(6-butyryl-4-methylpyridin-3-yl)-7-chloro-1-(2-fluoroethyl)-1,6-naphthyridin-2(1H)-one (500.0 mg, 1.29 mmol) in dioxane (5.0 mL) was added cyclopropanecarboxamide (131.7 mg, 1.55 mmol), Cs₂CO₃ (1260.1 mg, 3.87 mmol), Xphos Pd G3 (109.1 mg, 0.13 mmol) and Xphos (122.9 mg, 0.26 mmol) at room temperature under N₂. The resulting mixture was stirred at 100° C. for 2 h. After the reaction was completed, the mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (28/72, v/v) to afford N-(3-(6-butyryl-4-methylpyridin-3-yl)-1-(2-fluoroethyl)-2-oxo-1,2-dihydro-1,6-naphthyridin-7-yl)cyclopropanecarboxamide (200.0 mg, 36%) as a yellow solid. LCMS (ESI, m/z): [M+H]⁺=437.2.

### Step 4. Synthesis of N-(1-(2-fluoroethyl)-3-(6-(1-hydroxybutyl)-4-methylpyridin-3-yl)-2-oxo-1,2-dihydro-1,6-naphthyridin-7-yl)cyclopropanecarboxamide

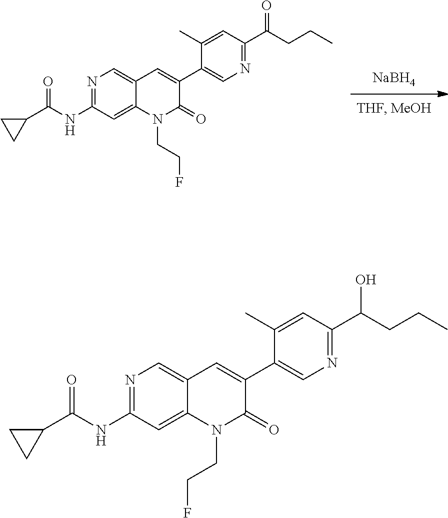 [SMILES: CCCC(=O)c1cc(C)c(-c2cc3cnc(NC(=O)C4CC4)cc3n(CCF)c2=O)cn1 ; "NaBH4" ; CCCC(O)c1cc(C)c(-c2cc3cnc(NC(=O)C4CC4)cc3n(CCF)c2=O)cn1]

To a solution of N-(3-(6-butyryl-4-methylpyridin-3-yl)-1-(2-fluoroethyl)-2-oxo-1,2-dihydro-1,6-naphthyridin-7-yl)cyclopropanecarboxamide (200.0 mg, 0.46 mmol) in THF/MeOH (2.0 mL/2.0 mL) was added NaBH₄ (20.8 mg, 0.55 mmol) at 0° C. under N₂. The resulting mixture was stirred at room temperature for 30 min under N₂. After the reaction was completed, the reaction mixture was quenched by water at 0° C. The mixture was concentrated under reduced pressure. The residue was purified by flash column chromatography with dichloromethane/methanol (94/6, v/v) to afford N-(1-(2-fluoroethyl)-3-(6-(1-hydroxybutyl)-4-methylpyridin-3-yl)-2-oxo-1,2-dihydro-1,6-naphthyridin-7-yl)cyclopropanecarboxamide (180.0 mg, 89%) as a yellow solid. LCMS (ESI, m/z): [M+H]⁺=439.2.

### Step 5. Chiral Separation of (S)—N-(1-(2-fluoroethyl)-3-(6-(1-hydroxybutyl)-4-methylpyridin-3-yl)-2-oxo-1,2-dihydro-1,6-naphthyridin-7-yl)cyclopropanecarboxamide and (R)—N-(1-(2-fluoroethyl)-3-(6-(1-hydroxybutyl)-4-methylpyridin-3-yl)-2-oxo-1,2-dihydro-1,6-naphthyridin-7-yl)cyclopropanecarboxamide (Compound 138 and 139)

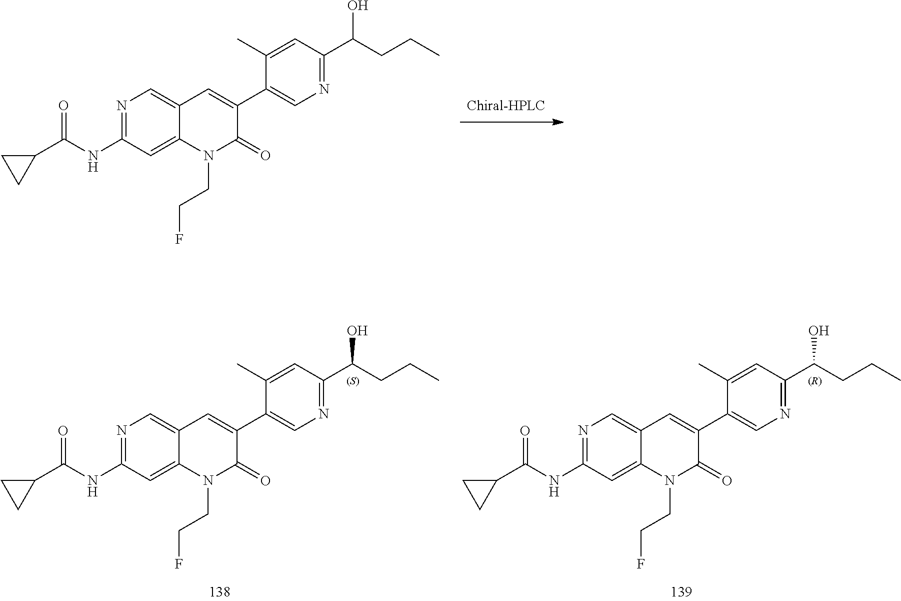

The racemic product of N-(1-(2-fluoroethyl)-3-(6-(1-hydroxybutyl)-4-methylpyridin-3-yl)-2-oxo-1,2-dihydro-1,6-naphthyridin-7-yl)cyclopropanecarboxamide (100.0 mg, 0.23 mmol) was separated by Prep-Chiral-HPLC with the following conditions (Column: CHIRAL ART Cellulose-SC, 2×25 cm, 5 m; Mobile Phase A: Hex-HPLC, Mobile Phase B: MeOH:DCM=1:1-HPLC; Flow rate: 20 mL/min; Gradient: 80% B to 80% B in 18 min; Wave Length: 254/220 nm; RT1(min): 9.27; RT2(min): 15.40) to afford N-(1-(2-fluoroethyl)-3-(6-(1-hydroxybutyl)-4-methylpyridin-3-yl)-2-oxo-1,2-dihydro-1,6-naphthyridin-7-yl)cyclopropanecarboxamide Enantiomer A (retention time 9.27 min, 35.5 mg, 70%) as a white solid and N-(1-(2-fluoroethyl)-3-(6-(1-hydroxybutyl)-4-methylpyridin-3-yl)-2-oxo-1,2-dihydro-1,6-naphthyridin-7-yl)cyclopropanecarboxamide Enantiomer B (retention time 15.40 min, 33.7 mg, 67%) as a white solid.

Enantiomer A: retention time 1: 9.27 min, LCMS (ESI, m/z): [M+H]⁺=439.3. ¹H NMR (400 MHz, DMSO-d₆): δ 11.15 (s, 1H), 8.73 (s, 1H), 8.36 (s, 2H), 8.08 (s, 1H), 7.50 (s, 1H), 5.63-5.39 (m, 1H), 4.85-4.82 (m, 1H), 4.73-4.70 (m, 1H), 4.66-4.60 (m, 1H), 4.55-4.40 (m, 2H), 2.25 (s, 3H), 2.10-2.00 (m, 1H), 1.77-1.60 (m, 2H), 1.41-1.34 (m, 2H), 0.92-0.84 (m, 7H).

Enantiomer B: retention time 2: 15.40 min, LCMS (ESI, m/z): [M+H]⁺=439.3. ¹H NMR (400 MHz, DMSO-d₆): δ 11.15 (s, 1H), 8.73 (s, 1H), 8.35 (s, 1H), 8.30 (s, 1H), 8.06 (s, 1H), 7.43 (s, 1H), 5.37 (s, 1H), 4.84-4.82 (m, 1H), 4.73-4.70 (m, 1H), 4.65-4.46 (m, 3H), 2.21 (s, 3H), 2.09-2.01 (m, 1H), 1.80-1.61 (m, 2H), 1.45-1.31 (m, 2H), 0.95-0.81 (m, 7H).

The absolute stereochemistry of enantiomers A and B was not assigned. The two enantiomeric structures that could be obtained from chiral separation of the enantiomeric mixture as described above are shown as Compounds 138 and 139 in Table 1.

### Example S57. Synthesis of (S)—N-(3-(6-(1-hydroxybutyl)-4-methylpyridin-3-yl)-1-(2-methoxyethyl)-2-oxo-1,2-dihydro-1,6-naphthyridin-7-yl)cyclopropanecarboxamide and (R)—N-(3-(6-(1-hydroxybutyl)-4-methylpyridin-3-yl)-1-(2-methoxyethyl)-2-oxo-1,2-dihydro-1,6-naphthyridin-7-yl)cyclopropanecarboxamide (Compound 140 and 141)

### Step 1. Synthesis of 3-bromo-7-chloro-1-(2-methoxyethyl)-1,6-naphthyridin-2(1H)-one

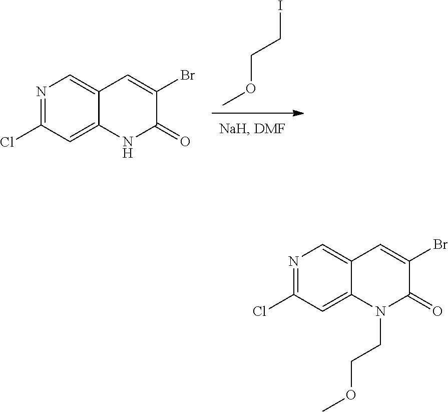

To a solution of 3-bromo-7-chloro-1,6-naphthyridin-2(1H)-one (1.0 g, 3.85 mmol) in DMF (30.0 mL) was added NaH (462.3 mg, 60%) at 0° C. under N₂. The resulting mixture was stirred at 0° C. for 1 h. Then 1-iodo-2-methoxyethane (1.1 g, 5.78 mmol) was added to the mixture at 0° C. under N₂. The resulting mixture was stirred at 60° C. for 3 h. After the reaction was completed, the resulting mixture was quenched with water at 0° C. and then extracted with ethyl acetate. The combined organic layers were washed with brine, dried over anhydrous sodium sulfate and filtered. The filtrate was concentrated under reduced pressure. The residue was purified by flash column chromatography with petroleum ether/ethyl acetate (20/80, v/v) to afford 3-bromo-7-chloro-1-(2-methoxyethyl)-1,6-naphthyridin-2(1H)-one (930.0 mg, 75%) as a yellow solid: [M+H]⁺=317.0.

### Step 2. Synthesis of 3-(6-butyryl-4-methylpyridin-3-yl)-7-chloro-1-(2-methoxyethyl)-1,6-naphthyridin-2(1H)-one

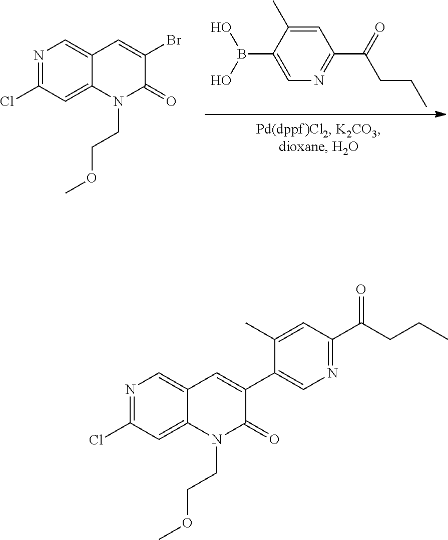 [SMILES: COCCn1c(=O)c(Br)cc2cnc(Cl)cc21 ; CCCC(=O)c1cc(C)c(B(O)O)cn1 ; CCCC(=O)c1cc(C)c(-c2cc3cnc(Cl)cc3n(CCOC)c2=O)cn1]

To a solution of 3-bromo-7-chloro-1-(2-methoxyethyl)-1,6-naphthyridin-2(1H)-one (500.0 mg, 1.57 mmol) in 1,4-dioxane/H₂O (15.0 mL/3.0 mL) was added (6-butyryl-4-methylpyridin-3-yl)boronic acid (488.9 mg, 2.36 mmol), K₂CO₃ (652.8 mg, 4.72 mmol) and Pd(dppf)Cl₂ (345.6 mg, 0.47 mmol) at room temperature under N₂. The resulting mixture was stirred at 80° C. for 2 h. After the reaction was completed, the mixture was concentrated under reduced pressure. The residue was purified by flash column chromatography with petroleum ether/ethyl acetate (77/23, v/v) to afford 3-(6-butyryl-4-methylpyridin-3-yl)-7-chloro-1-(2-methoxyethyl)-1,6-naphthyridin-2(1H)-one (330.0 mg, 52%) as a white solid. LCMS (ESI, m/z): [M+H]⁺=400.1.

### Step 3. Synthesis of N-(3-(6-butyryl-4-methylpyridin-3-yl)-1-(2-methoxyethyl)-2-oxo-1,2-dihydro-1,6-naphthyridin-7-yl)cyclopropanecarboxamide

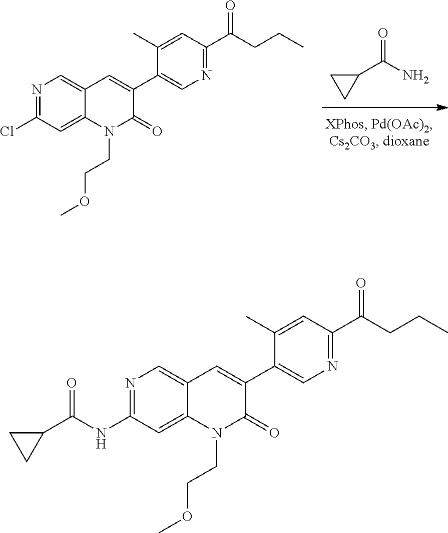

To a solution of 3-(6-butyryl-4-methylpyridin-3-yl)-7-chloro-1-(2-methoxyethyl)-1,6-naphthyridin-2(1H)-one (300.0 mg, 0.75 mmol) in dioxane (10.0 mL) was added cyclopropanecarboxamide (95.8 mg, 1.13 mmol), Cs₂CO₃ (733.3 mg, 2.25 mmol), XPhos (143.1 mg, 0.30 mmol) and Pd(OAc)₂ (33.7 mg, 0.15 mmol) at room temperature under N₂. The resulting mixture was stirred at 100° C. for 2 h. After the reaction was completed, the resulting mixture was filtered. The filtrate was concentrated under reduced pressure. The residue was purified by flash chromatography with petroleum ether/ethyl acetate (66/34, v/v) to afford N-(3-(6-butyryl-4-methylpyridin-3-yl)-1-(2-methoxyethyl)-2-oxo-1,2-dihydro-1,6-naphthyridin-7-yl)cyclopropanecarboxamide (170.0 mg, 50%) as a yellow solid. LCMS (ESI, m/z): [M+H]⁺=449.2.

### Step 4. Synthesis of N-(3-(6-(1-hydroxybutyl)-4-methylpyridin-3-yl)-1-(2-methoxyethyl)-2-oxo-1,2-dihydro-1,6-naphthyridin-7-yl)cyclopropanecarboxamide

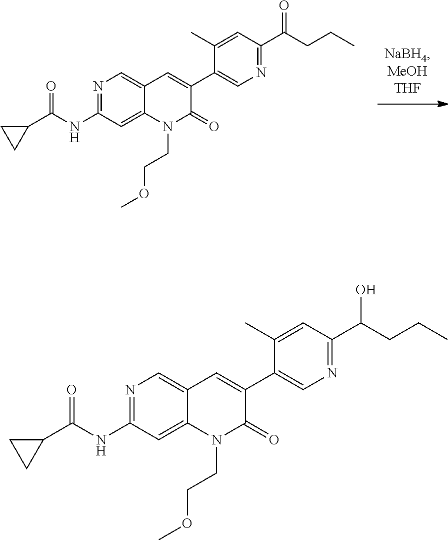 [SMILES: CCCC(=O)c1cc(C)c(-c2cc3cnc(NC(=O)C4CC4)cc3n(CCOC)c2=O)cn1 ; CCCC(O)c1cc(C)c(-c2cc3cnc(NC(=O)C4CC4)cc3n(CCOC)c2=O)cn1]

To a solution of N-(3-(6-butyryl-4-methylpyridin-3-yl)-1-(2-methoxyethyl)-2-oxo-1,2-dihydro-1,6-naphthyridin-7-yl)cyclopropanecarboxamide (170.0 mg, 0.38 mmol) in THF/MeOH (2.0 mL/2.0 m) was added NaBH₄ (57.4 mg, 1.52 mmol) at room temperature. The resulting mixture was stirred at room temperature for 1 h. After the reaction was completed, the reaction mixture was quenched by water at 0° C. The mixture was concentrated under reduced pressure. The residue was purified by reverse phase flash chromatography with acetonitrile/water (28/72, v/v) to afford N-(3-(6-(1-hydroxybutyl)-4-methylpyridin-3-yl)-1-(2-methoxyethyl)-2-oxo-1,2-dihydro-1,6-naphthyridin-7-yl)cyclopropanecarboxamide (95.0 mg, 55%) as a yellow solid. LCMS (ESI, m/z): [M+H]⁺=451.2.

### Step 5. Chiral Separation of (S)—N-(3-(6-(1-hydroxybutyl)-4-methylpyridin-3-yl)-1-(2-methoxyethyl)-2-oxo-1,2-dihydro-1,6-naphthyridin-7-yl)cyclopropanecarboxamide and (R)—N-(3-(6-(1-hydroxybutyl)-4-methylpyridin-3-yl)-1-(2-methoxyethyl)-2-oxo-1,2-dihydro-1,6-naphthyridin-7-yl)cyclopropanecarboxamide (Compound 140 and 141)

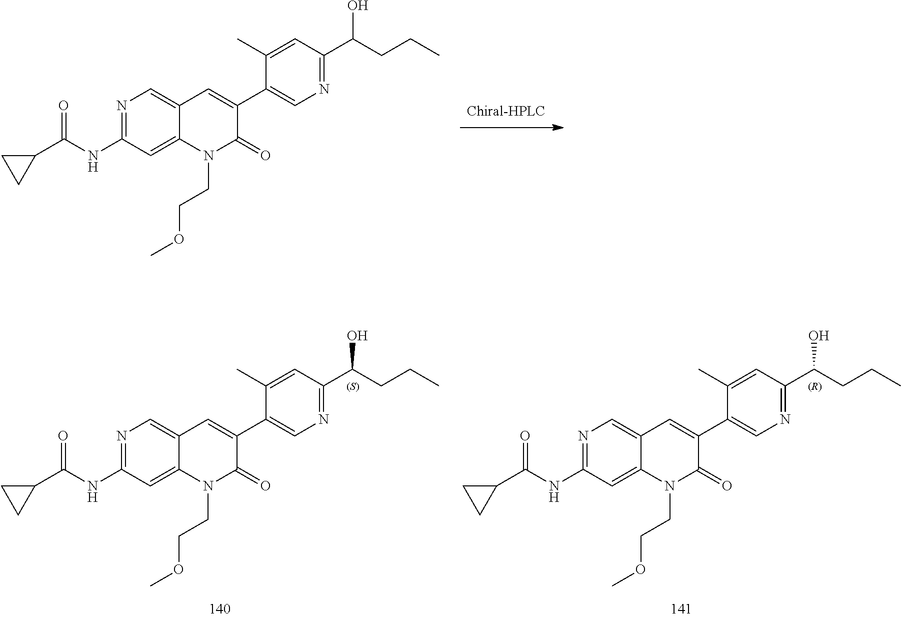

The racemic product of N-(3-(6-(1-hydroxybutyl)-4-methylpyridin-3-yl)-1-(2-methoxyethyl)-2-oxo-1,2-dihydro-1,6-naphthyridin-7-yl)cyclopropanecarboxamide (95.0 mg, 0.38 mmol) was separated by Prep-Chiral-HPLC with the following conditions (Column: Lux Sum Cellulose-4, 2.12×25 cm, 5 m; Mobile Phase A: Hex(0.5% 2M NH₃-MeOH)-HPLC, Mobile Phase B: MeOH:EtOH=1:1-HPLC; Flow rate: 20 mL/min; Gradient: 80% B to 80% B in 15 min; Wave Length: 254/220 nm; RT1(min): 8.55; RT2(min): 11.87) to afford N-(3-(6-(1-hydroxybutyl)-4-methylpyridin-3-yl)-1-(2-methoxyethyl)-2-oxo-1,2-dihydro-1,6-naphthyridin-7-yl)cyclopropanecarboxamide Enantiomer A (retention time 8.55 min, 28.9 mg, 60%) as a white solid and N-(3-(6-(1-hydroxybutyl)-4-methylpyridin-3-yl)-1-(2-methoxyethyl)-2-oxo-1,2-dihydro-1,6-naphthyridin-7-yl)cyclopropanecarboxamide Enantiomer B (retention time 11.87 min, 31.1 mg, 65%) as a white solid.

Enantiomer A: retention time 1: 8.55 min, LCMS (ESI, m/z): [M+H]⁺=451.3. ¹H NMR (400 MHz, DMSO-d₆): δ 11.16 (s, 1H), 8.71 (s, 1H), 8.34 (s, 1H), 8.29 (s, 1H), 8.02 (s, 1H), 7.42 (s, 1H), 5.33 (s, 1H), 4.61-4.58 (m, 1H), 4.38-4.35 (m, 2H), 3.66-3.63 (m, 2H), 3.26 (s, 3H), 2.21 (s, 3H), 2.12-2.05 (m, 1H), 1.77-1.70 (m, 1H), 1.65-1.60 (m, 1H), 1.42-1.36 (m, 2H), 0.92-0.84 (m, 7H).

Enantiomer B: retention time 2: 11.87 min, LCMS (ESI, m/z): [M+H]⁺=451.2. ¹H NMR (400 MHz, DMSO-d₆): δ 11.13 (s, 1H), 8.71 (s, 1H), 8.35 (s, 1H), 8.30 (s, 1H), 8.03 (s, 1H), 7.43 (s, 1H), 5.48-5.31 (m, 1H), 4.62-4.59 (m, 1H), 4.38-4.35 (m, 2H), 3.66-3.64 (m, 2H), 3.26 (s, 3H), 2.21 (s, 3H), 2.10-2.05 (m, 1H), 1.76-1.71 (m, 1H), 1.66-1.61 (m, 1H), 1.42-1.35 (m, 2H), 0.93-0.84 (m, 7H).

The absolute stereochemistry of enantiomers A and B was not assigned. The two enantiomeric structures that could be obtained from chiral separation of the enantiomeric mixture as described above are shown as Compounds 140 and 141 in Table 1.

### Example S58. Synthesis of (S)—N-(1-(cyclopropylmethyl)-3-(6-(1-hydroxybutyl)-4-methylpyridin-3-yl)-2-oxo-1,2-dihydro-1,6-naphthyridin-7-yl)cyclopropanecarboxamide and (R)—N-(1-(cyclopropylmethyl)-3-(6-(1-hydroxybutyl)-4-methylpyridin-3-yl)-2-oxo-1,2-dihydro-1,6-naphthyridin-7-yl)cyclopropanecarboxamide (Compound 142 and 143)

### Step 1. Synthesis of 3-bromo-7-chloro-1-(cyclopropylmethyl)-1,6-naphthyridin-2(1H)-one

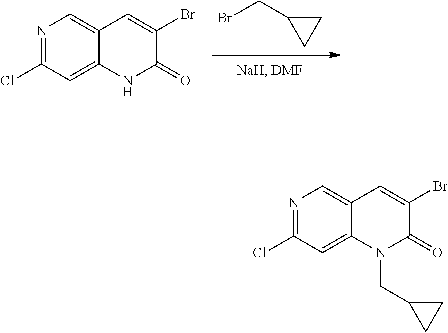

To a solution of 3-bromo-7-chloro-1,6-naphthyridin-2(1H)-one (1.5 g, 5.78 mmol) in DMF (15.0 mL) was added NaH (0.5 g, 60%) at 0° C. under N₂. The resulting mixture was stirred at 0° C. for 30 min. Then (bromomethyl)cyclopropane (3.9 g, 28.91 mmol) was added to the mixture at room temperature. The resulting mixture was stirred at room temperature for additional 30 min and then stirred at 50° C. for 4 h. After the reaction was completed, the reaction mixture was quenched H₂O at 0° C. and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (77/23, v/v) to afford 3-bromo-7-chloro-1-(cyclopropylmethyl)-1,6-naphthyridin-2(1H)-one (0.9 g, 50%) as a yellow solid. LCMS (ESI, m/z): [M+H]⁺=313.0. ¹H NMR (400 MHz, DMSO-d₆): δ 8.85-8.74 (m, 1H), 8.70-8.64 (m, 1H), 7.85-7.80 (m, 1H), 4.22 (d, J=6.8 Hz, 2H), 1.39-1.7 (m, 1H), 0.39-0.56 (m, 4H).

### Step 2. Synthesis of 3-(6-butyryl-4-methylpyridin-3-yl)-7-chloro-1-(cyclopropylmethyl)-1,6-naphthyridin-2(1H)-one

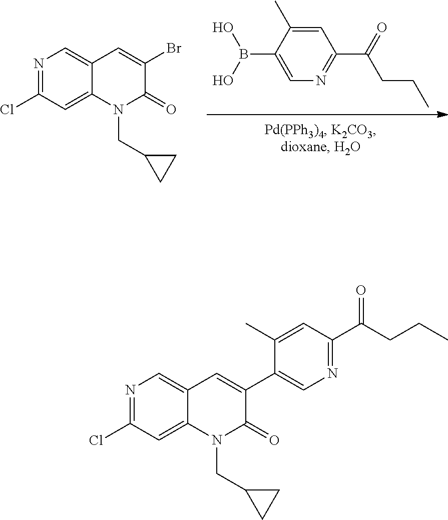 [SMILES: O=c1c(Br)cc2cnc(Cl)cc2n1CC1CC1 ; CCCC(=O)c1cc(C)c(B(O)O)cn1 ; CCCC(=O)c1cc(C)c(-c2cc3cnc(Cl)cc3n(CC3CC3)c2=O)cn1]

To a solution of 3-bromo-7-chloro-1-(cyclopropylmethyl)-1,6-naphthyridin-2(1H)-one (840.0 mg, 2.68 mmol) in dioxane/H₂O (10.0 mL/2.0 mL) was added (6-butyryl-4-methylpyridin-3-yl)boronic acid (554.6 mg, 2.68 mmol), K₂CO₃ (1110.7 mg, 8.04 mmol) and Pd(PPh₃)₄ (619.1 mg, 0.54 mmol) at room temperature under N₂. The resulting mixture was stirred at 80° C. for 16 h under N₂. After the reaction was completed, the mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (50/50, v/v) to afford 3-(6-butyryl-4-methylpyridin-3-yl)-7-chloro-1-(cyclopropylmethyl)-1,6-naphthyridin-2(1H)-one (540.0 mg, 50%) as a yellow solid. LCMS (ESI, m/z): [M+H]⁺=396.1.

### Step 3. Synthesis of N-(3-(6-butyryl-4-methylpyridin-3-yl)-1-(cyclopropylmethyl)-2-oxo-1,2-dihydro-1,6-naphthyridin-7-yl)cyclopropanecarboxamide

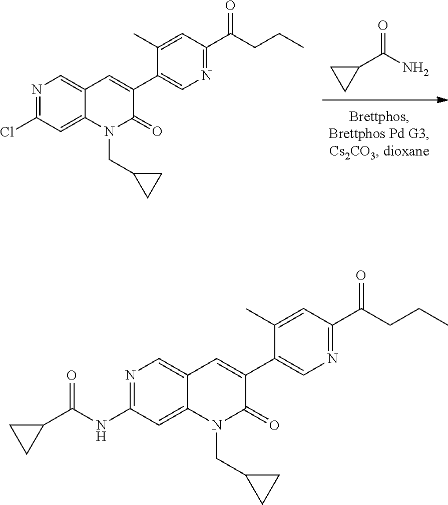

To a solution of 3-(6-butyryl-4-methylpyridin-3-yl)-7-chloro-1-(cyclopropylmethyl)-1,6-naphthyridin-2(1H)-one (500.0 mg, 1.26 mmol) in dioxane (5.0 mL) was added cyclopropanecarboxamide (161.2 mg, 1.89 mmol), Cs₂CO₃ (1234.5 mg, 3.79 mmol), Brettphos (135.6 mg, 0.25 mmol) and Brettphos Pd G3 (114.5 mg, 0.13 mmol) at room temperature under N₂. The resulting mixture was stirred at 100° C. for 2 h under N₂. After the reaction was completed, the mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (38/62, v/v) to afford N-(3-(6-butyryl-4-methylpyridin-3-yl)-1-(cyclopropylmethyl)-2-oxo-1,2-dihydro-1,6-naphthyridin-7-yl)cyclopropanecarboxamide (280.0 mg, 49%) as a yellow solid. LCMS (ESI, m/z): [M+H]⁺=445.2.

### Step 4. Synthesis of N-(1-(cyclopropylmethyl)-3-(6-(1-hydroxybutyl)-4-methylpyridin-3-yl)-2-oxo-1,2-dihydro-1,6-naphthyridin-7-yl)cyclopropanecarboxamide

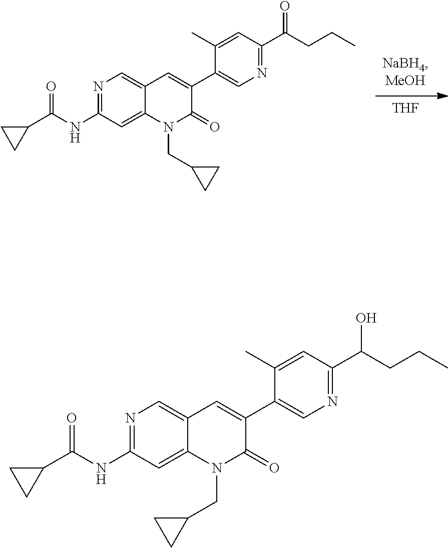 [SMILES: CCCC(=O)c1cc(C)c(-c2cc3cnc(NC(=O)C4CC4)cc3n(CC3CC3)c2=O)cn1 ; CCCC(O)c1cc(C)c(-c2cc3cnc(NC(=O)C4CC4)cc3n(CC3CC3)c2=O)cn1 ; "THF"]

To a solution of N-(3-(6-butyryl-4-methylpyridin-3-yl)-1-(cyclopropylmethyl)-2-oxo-1,2-dihydro-1,6-naphthyridin-7-yl)cyclopropanecarboxamide (230.0 mg, 0.52 mmol) in THF/MeOH (2.0 mL/2.0 mL) was added NaBH₄ (23.5 mg, 0.62 mmol) at 0° C. under N₂. The resulting mixture was stirred at room temperature for 30 min. After the reaction was completed, the reaction mixture was quenched by water at 0° C. The mixture was concentrated under reduced pressure. The residue was purified by flash column chromatography with dichloromethane/methanol (88/12, v/v) to afford N-(1-(cyclopropylmethyl)-3-(6-(1-hydroxybutyl)-4-methylpyridin-3-yl)-2-oxo-1,2-dihydro-1,6-naphthyridin-7-yl)cyclopropanecarboxamide (200.0 mg, 86%) as a yellow solid. LCMS (ESI, m/z): [M+H]⁺=447.2.

### Step 5. Chiral Separation of (S)—N-(1-(cyclopropylmethyl)-3-(6-(1-hydroxybutyl)-4-methylpyridin-3-yl)-2-oxo-1,2-dihydro-1,6-naphthyridin-7-yl)cyclopropanecarboxamide and (R)—N-(1-(cyclopropylmethyl)-3-(6-(1-hydroxybutyl)-4-methylpyridin-3-yl)-2-oxo-1,2-dihydro-1,6-naphthyridin-7-yl)cyclopropanecarboxamide (Compound 142 and 143)

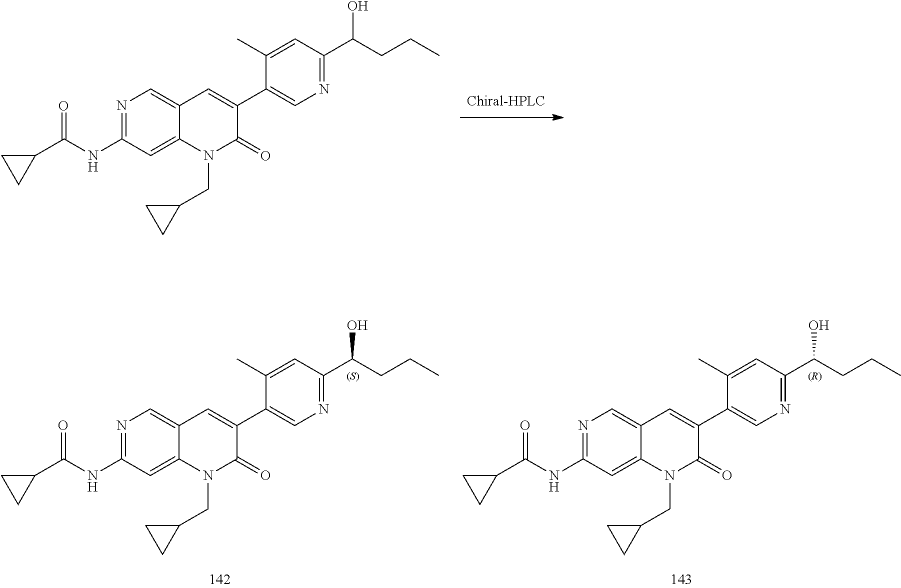

The racemic product of N-(1-(cyclopropylmethyl)-3-(6-(1-hydroxybutyl)-4-methylpyridin-3-yl)-2-oxo-1,2-dihydro-1,6-naphthyridin-7-yl)cyclopropanecarboxamide (100.0 mg, 0.22 mmol) was separated by Prep-Chiral-HPLC with the following conditions (Column: Lux 5 um Cellulose-4, 2.12×25 cm, 5 m; Mobile Phase A: Hex(0.5% 2M NH₃-MeOH)-HPLC, Mobile Phase B: MeOH:EtOH=1:1-HPLC; Flow rate: 20 mL/min; Gradient: 80% B to 80% B in 10 min; Wave Length: 254/220 nm; RT1(min): 6.73; RT2(min): 8.24) to afford N-(1-(cyclopropylmethyl)-3-(6-(1-hydroxybutyl)-4-methylpyridin-3-yl)-2-oxo-1,2-dihydro-1,6-naphthyridin-7-yl)cyclopropanecarboxamide Enantiomer A (retention time 6.73 min, 12.5 mg, 25%) as a white solid and N-(1-(cyclopropylmethyl)-3-(6-(1-hydroxybutyl)-4-methylpyridin-3-yl)-2-oxo-1,2-dihydro-1,6-naphthyridin-7-yl)cyclopropanecarboxamide Enantiomer B (retention time 8.24 min, 10.2 mg, 20%) as a white solid.

Enantiomer A: retention time 1: 6.73 min, LCMS (ESI, m/z): [M+H]⁺=447.3. ¹H NMR (400 MHz, DMSO-d₆): δ 11.19 (s, 1H), 8.73 (s, 1H), 8.40 (s, 1H), 8.28 (s, 1H), 8.02 (s, 1H), 7.40 (s, 1H), 5.29 (d, J=5.2 Hz, 1H), 4.61-4.56 (m, 1H), 4.12 (d, J=7.2 Hz, 2H), 2.20 (s, 3H), 2.11-2.07 (m, 1H), 1.76-1.70 (m, 1H), 1.66-1.59 (m, 1H), 1.45-1.35 (m, 2H), 1.27-1.22 (m, 1H), 0.92-0.84 (m, 7H), 0.51-0.48 (m, 4H).

Enantiomer B: retention time 2: 8.24 min, LCMS (ESI, m/z): [M+H]⁺=447.2. ¹H NMR (400 MHz, DMSO-d₆): δ 11.15 (s, 1H), 8.73 (s, 1H), 8.40 (s, 1H), 8.28 (s, 1H), 8.02 (s, 1H), 7.39 (s, 1H), 5.30 (s, 1H), 4.61-4.56 (m, 1H), 4.12 (d, J=6.8 Hz, 2H), 2.20 (s, 3H), 2.10-2.04 (m, 1H), 1.79-1.70 (m, 1H), 1.67-1.58 (m, 1H), 1.42-1.36 (m, 2H), 1.28-1.20 (m, 1H), 0.92-0.82 (m, 7H), 0.51-0.46 (m, 4H).

The absolute stereochemistry of enantiomers A and B was not assigned. The two enantiomeric structures that could be obtained from chiral separation of the enantiomeric mixture as described above are shown as Compounds 142 and 143 in Table 1.

### Example S59. Synthesis of (R)—N-(3-(6-(1-hydroxypropyl)-4-methylpyridin-3-yl)-1-methyl-2-oxo-1,2-dihydro-1,6-naphthyridin-7-yl)acetamide and (S)—N-(3-(6-(1-hydroxypropyl)-4-methylpyridin-3-yl)-1-methyl-2-oxo-1,2-dihydro-1,6-naphthyridin-7-yl)acetamide (Compounds 144 and 145)

### Step 1. Synthesis of N-(1-methyl-3-(4-methyl-6-propionylpyridin-3-yl)-2-oxo-1,2-dihydro-1,6-naphthyridin-7-yl)acetamide

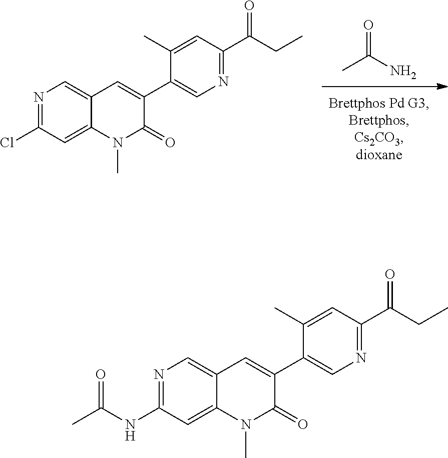

To a solution of 7-chloro-1-methyl-3-(4-methyl-6-propionylpyridin-3-yl)-1,6-naphthyridin-2(1H)-one (500.0 mg, 1.46 mmol) in dioxane (5.0 mL) was added acetamide (129.6 mg, 2.19 mmol), Cs₂CO₃ (1191.5 mg, 3.65 mmol), Brettphos Pd G3 (132.6 mg, 0.14 mmol) and Brettphos (157.0 mg, 0.29 mmol) at room temperature under N₂. The resulting mixture was stirred at 80° C. for 16 h under N₂. After the reaction was completed, the resulting mixture was filtered. The filtrate was concentrated under reduced pressure. The residue was purified by flash chromatography with petroleum ether/ethyl acetate (80/20, v/v) to afford N-(1-methyl-3-(4-methyl-6-propionylpyridin-3-yl)-2-oxo-1,2-dihydro-1,6-naphthyridin-7-yl)acetamide (360.0 mg, 67%) as a white solid. LCMS (ESI, m/z): [M+H]⁺=365.2.

### Step 2. Synthesis of N-(3-(6-(1-hydroxypropyl)-4-methylpyridin-3-yl)-1-methyl-2-oxo-1,2-dihydro-1,6-naphthyridin-7-yl)acetamide

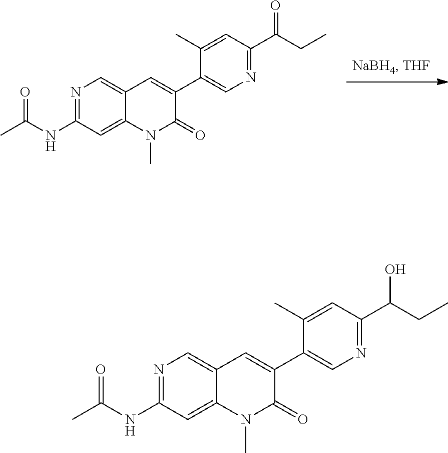

To a solution of N-(1-methyl-3-(4-methyl-6-propionylpyridin-3-yl)-2-oxo-1,2-dihydro-1,6-naphthyridin-7-yl)acetamide (150.0 mg, 0.41 mmol) in THF (5.0 mL) was added NaBH₄ (17.1 mg, 0.45 mmol) at room temperature. The resulting mixture was stirred at room temperature for 2 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/MeOH (94/6, v/v) to afford N-(3-(6-(1-hydroxypropyl)-4-methylpyridin-3-yl)-1-methyl-2-oxo-1,2-dihydro-1,6-naphthyridin-7-yl)acetamide (100.0 mg, 66%) as a white solid. LCMS (ESI, m/z): [M+H]⁺=367.2.

### Step 3. Chiral Separation of (R)—N-(3-(6-(1-hydroxypropyl)-4-methylpyridin-3-yl)-1-methyl-2-oxo-1,2-dihydro-1,6-naphthyridin-7-yl)acetamide and (S)—N-(3-(6-(1-hydroxypropyl)-4-methylpyridin-3-yl)-1-methyl-2-oxo-1,2-dihydro-1,6-naphthyridin-7-yl)acetamide (Compounds 144 and 145)

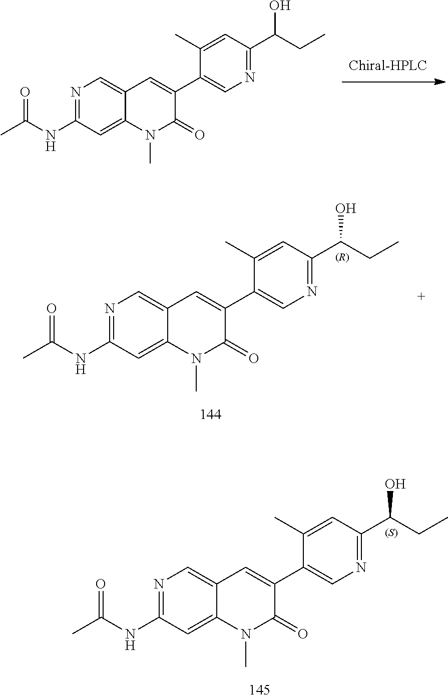

The racemic product of N-(3-(6-(1-hydroxypropyl)-4-methylpyridin-3-yl)-1-methyl-2-oxo-1,2-dihydro-1,6-naphthyridin-7-yl)acetamide (100.0 mg, 0.27 mmol) was separated by Prep-Chiral-HPLC with the following conditions (Column: CHIRAL ART Amylose-SA, 2×25 cm, 5 m; Mobile Phase A: MtBE (0.5% 2M NH₃-MeOH)-HPLC, Mobile Phase B: EtOH:DCM=1:1-HPLC; Flow rate: 20 mL/min; Gradient: 20% B to 20% B in 14.5 min; Wave Length: 220/254 nm; RT1(min): 9.88; RT2(min): 12.15) to afford N-(3-(6-(1-hydroxypropyl)-4-methylpyridin-3-yl)-1-methyl-2-oxo-1,2-dihydro-1,6-naphthyridin-7-yl)acetamide Enantiomer A (retention time 9.88 min, 48.8 mg, 97%) as a white solid and N-(3-(6-(1-hydroxypropyl)-4-methylpyridin-3-yl)-1-methyl-2-oxo-1,2-dihydro-1,6-naphthyridin-7-yl)acetamide Enantiomer B (retention time 12.15 min, 49.0 mg, 98%) as a white solid.

Enantiomer A: retention time 1: 9.88 min, LCMS (ESI, m/z): [M+H]⁺=367.3. ¹H NMR (400 MHz, DMSO-d₆): δ 10.83 (s, 1H), 8.70 (s, 1H), 8.27-8.24 (m, 2H), 8.00 (s, 1H), 7.40 (s, 1H), 5.33-5.28 (m, 1H), 4.54-4.49 (m, 1H), 3.61 (s, 3H), 2.21-2.17 (m, 6H), 1.86-1.79 (m, 1H), 1.70-1.62 (m, 1H), 0.92-0.88 (m, 3H).

Enantiomer B: retention time 2: 12.15 min, LCMS (ESI, m/z): [M+H]⁺=367.3. ¹H NMR (400 MHz, DMSO-d₆): δ 10.83 (s, 1H), 8.70 (s, 1H), 8.27-8.24 (m, 2H), 8.00 (s, 1H), 7.40 (s, 1H), 5.31 (d, J=4.4 Hz, 1H), 4.53-4.51 (m, 1H), 3.61 (s, 3H), 2.21-2.17 (m, 6H), 1.86-1.70 (m, 1H), 1.67-1.62 (m, 1H), 0.92-0.88 (m, 3H).

The absolute stereochemistry of enantiomers A and B was not assigned. The two enantiomeric structures that could be obtained from chiral separation of the enantiomeric mixture as described above are shown as Compounds 144 and 145 in Table 1.

### Example S60. Synthesis of N-(3-{6-[(1S)-1-hydroxy-2-methoxyethyl]-4-methylpyridin-3-yl}-1-methyl-2-oxo-1,6-naphthyridin-7-yl)cyclopropanecarboxamide and N-(3-{6-[(1R)-1-hydroxy-2-methoxyethyl]-4-methylpyridin-3-yl}-1-methyl-2-oxo-1,6-naphthyridin-7-yl)cyclopropanecarboxamide (Compounds 146 and 147)

### Step 1. Synthesis of 1-(5-bromo-4-methylpyridin-2-yl)-2-methoxyethanone

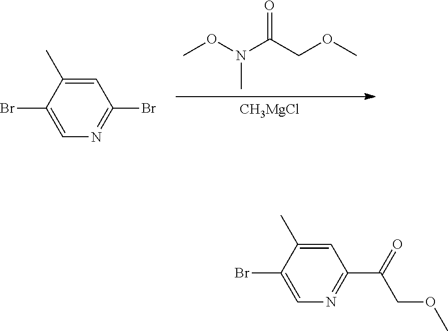

To a stirred mixture of 2,5-dibromo-4-methylpyridine (4.2 g, 16.89 mmol) in toluene (20.0 mL) was added isopropylmagnesium chloride (1.7 g, 16.89 mmol) at 0° C. under N₂. The resulting mixture was stirred at 0° C. for 1.5 h under N₂. Then N,2-dimethoxy-N-methylacetamide (1.5 g, 11.26 mmol) was added to the mixture at 0° C. under N₂. The resulting mixture was stirred at 0° C. for additional 3 h under N₂. After the reaction was completed, the reaction was quenched with NH₄Cl (aq.) and then extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (5/1, v/v) to afford 1-(5-bromo-4-methylpyridin-2-yl)-2-methoxyethanone (900.0 mg, 32%) as a white solid. LCMS (ESI, m/z): [M+H]⁺=244.1.

### Step 2. Synthesis of 2-methoxy-1-[4-methyl-5-(4,4,5,5-tetramethyl-1,3,2-dioxaborolan-2-yl)pyridin-2-yl]ethanone

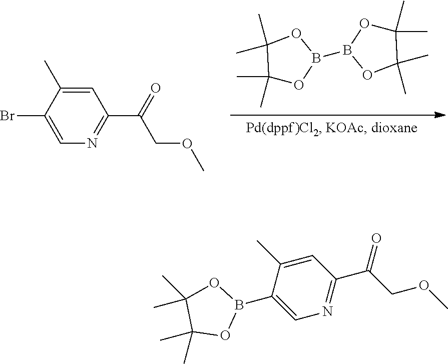

To a stirred mixture of 1-(5-bromo-4-methylpyridin-2-yl)-2-methoxyethanone (500.0 mg, 2.04 mmol) and 4,4,4′,4′,5,5,5′,5′-octamethyl-2,2′-bi(1,3,2-dioxaborolane) (1.5 g, 6.14 mmol) in 1,4-dioxane (10.0 mL) were added Pd(dppf)Cl₂ (299.7 mg, 0.41 mmol) and CH₃COOK (603.1 mg, 6.14 mmol) at room temperature. The resulting mixture was stirred at 100° C. for 16 h under N₂. After the reaction was completed, the mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/CH₃OH (10/1, v/v) to afford 2-methoxy-1-[4-methyl-5-(4,4,5,5-tetramethyl-1,3,2-dioxaborolan-2-yl)pyridin-2-yl]ethanone (270.0 mg, 45%) as a black solid. LCMS (ESI, m/z): [M+H]⁺=292.2.

### Step 3. Synthesis of 7-chloro-3-[6-(2-methoxyacetyl)-4-methylpyridin-3-yl]-1-methyl-1,6-naphthyridin-2-one

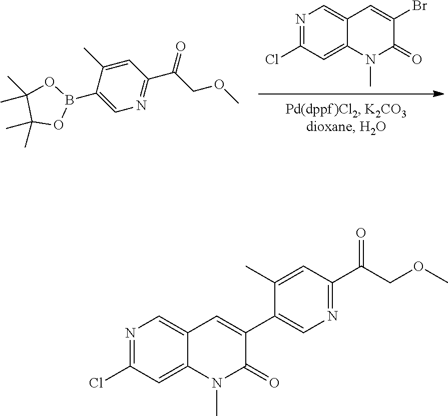

To a mixture of 2-methoxy-1-[4-methyl-5-(4,4,5,5-tetramethyl-1,3,2-dioxaborolan-2-yl)pyridin-2-yl]ethanone (150.0 mg, 0.51 mmol) and 3-bromo-7-chloro-1-methyl-1,6-naphthyridin-2-one (140.9 mg, 0.55 mmol) in 1,4-dioxane (5.0 mL) and H₂O (1.0 mL) were added Pd(dppf)Cl₂ (37.7 mg, 0.05 mmol) and K₂CO₃ (213.6 mg, 1.54 mmol) at room temperature. The resulting mixture was stirred at 100° C. for 16 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford 7-chloro-3-[6-(2-methoxyacetyl)-4-methylpyridin-3-yl]-1-methyl-1,6-naphthyridin-2-one (170.0 mg, 85%) as a black solid. LCMS (ESI, m/z): [M+H]⁺=358.1.

### Step 4. Synthesis of N-{3-[6-(2-methoxyacetyl)-4-methylpyridin-3-yl]-1-methyl-2-oxo-1,6-naphthyridin-7-yl}cyclopropanecarboxamide

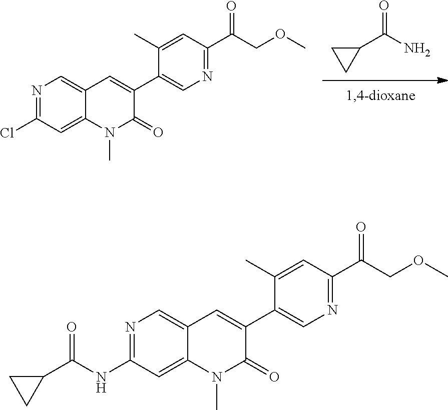

To a mixture 7-chloro-3-[6-(2-methoxyacetyl)-4-methylpyridin-3-yl]-1-methyl-1,6-naphthyridin-2-one (170.0 mg, 0.47 mmol) in 1,4-dioxane (5.0 mL) was added cyclopropanecarboxamide (202.1 mg, 2.37 mmol), Cs₂CO₃ (464.4 mg, 1.42 mmol), EPhos Pd G4 (25.4 mg, 0.05 mmol) and EPhos (50.8 mg, 0.09 mmol) at room temperature. The resulting mixture was stirred at 100° C. for 16 h under N₂. After the reaction was completed, the reaction mixture was diluted with H₂O and extracted with EtOAc. The combined organic layers were washed with brine and dried over anhydrous Na₂SO₄. After filtration, the filtrate was concentrated under reduced pressure. The residue was purified by flash column chromatography with dichloromethane/methanol (10/1, v/v) to afford N-{3-[6-(2-methoxyacetyl)-4-methylpyridin-3-yl]-1-methyl-2-oxo-1,6-naphthyridin-7-yl}cyclopropanecarboxamide (170.0 mg, 66%) as a yellow solid LCMS (ESI, m/z): [M+H]⁺=407.2.

### Step 5. Synthesis of N-{3-[6-(2-methoxyacetyl)-4-methylpyridin-3-yl]-1-methyl-2-oxo-1,6-naphthyridin-7-yl}cyclopropanecarboxamide

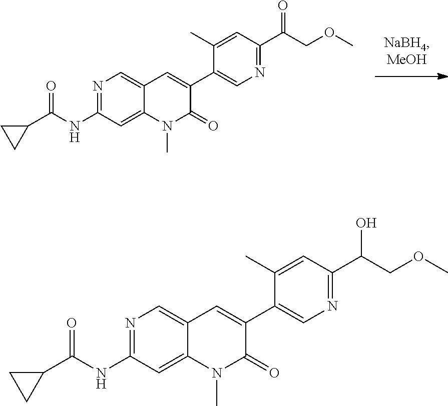 [SMILES: COCC(=O)c1cc(C)c(-c2cc3cnc(NC(=O)C4CC4)cc3n(C)c2=O)cn1 ; COCC(O)c1cc(C)c(-c2cc3cnc(NC(=O)C4CC4)cc3n(C)c2=O)cn1]

To a mixture N-{3-[6-(2-methoxyacetyl)-4-methylpyridin-3-yl]-1-methyl-2-oxo-1,6-naphthyridin-7-yl}cyclopropanecarboxamide (150.0 mg, 0.36 mmol) in MeOH (2.0 mL) was added NaBH₄ (69.8 mg, 1.84 mmol) at 0° C. The resulting mixture was stirred at room temperature for 2 h under N₂. After the reaction was completed, the reaction was diluted with H₂O and extracted with EtOAc. The combined organic layers were washed with brine and dried over anhydrous Na₂SO₄. After filtration, the filtrate was concentrated under reduced pressure. The residue was purified by flash column chromatography with dichloromethane/methanol (10/1, v/v) to afford N-{3-[6-(1-hydroxy-2-methoxyethyl)-4-methylpyridin-3-yl]-1-methyl-2-oxo-1,6-naphthyridin-7-yl}cyclopropanecarboxamide (70.0 mg, 44%) as a white solid. LCMS (ESI, m/z): [M+H]⁺=409.2.

### Step 6. Chiral Separation of N-(3-{6-[(1S)-1-hydroxy-2-methoxyethyl]-4-methylpyridin-3-yl}-1-methyl-2-oxo-1,6-naphthyridin-7-yl)cyclopropanecarboxamide and N-(3-{6-[(1R)-1-hydroxy-2-methoxyethyl]-4-methylpyridin-3-yl}-1-methyl-2-oxo-1,6-naphthyridin-7-yl)cyclopropanecarboxamide (Compounds 146 and 147)

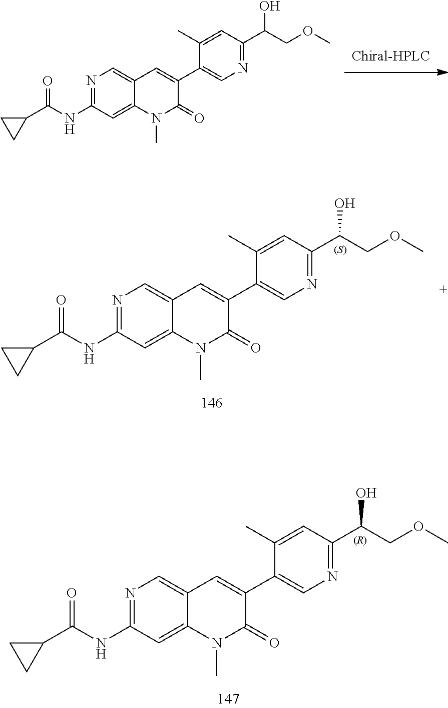

The racemic product of N-{3-[6-(1-hydroxy-2-methoxyethyl)-4-methylpyridin-3-yl]-1-methyl-2-oxo-1,6-naphthyridin-7-yl}cyclopropanecarboxamide (70.0 mg, 0.17 mmol) was separated by Prep-Chiral-HPLC with the following conditions (Column: CHIRAL ART Cellulose-SC, 2×25 cm, 5 m; Mobile Phase A: Hex (0.5% 2M NH₃-MeOH)-HPLC, Mobile Phase B: EtOH:DCM=1:1-HPLC; Flow rate: 20 mL/min; Gradient: 60% B to 60% B in 20 min; Wave Length: 220/254 nm; RT1 (min): 13.52; RT2 (min): 17.48) to afford N-(3-{6-[1-hydroxy-2-methoxyethyl]-4-methylpyridin-3-yl}-1-methyl-2-oxo-1,6-naphthyridin-7-yl)cyclopropanecarboxamide Enantiomer A (retention time 13.52 min, 15.3 mg, 44%) as a white solid and N-(3-{6-[1-hydroxy-2-methoxyethyl]-4-methylpyridin-3-yl}-1-methyl-2-oxo-1,6-naphthyridin-7-yl)cyclopropanecarboxamide Enantiomer B (retention time 17.48 min, 14.1 mg, 40%) as a white solid

Enantiomer A: retention time 1: 13.52 min, LCMS (ESI, m/z): [M+H]⁺=409.0. ¹H NMR (400 MHz, DMSO-d₆): δ 11.15 (s, 1H), 8.71 (s, 1H), 8.29-8.26 (m, 2H), 8.01 (s, 1H), 7.43 (s, 1H), 5.54 (d, J=4.0 Hz, 1H), 4.78-4.73 (m, 1H), 3.69-3.65 (m, 1H), 3.60 (s, 3H), 3.56-3.51 (m, 1H), 3.32 (s, 3H), 2.21 (s, 3H), 2.13-2.06 (m, 1H), 0.91-0.84 (m, 4H).

Enantiomer B: retention time 2: 17.48 min, LCMS (ESI, m/z): [M+H]⁺=409.0. ¹H NMR (400 MHz, DMSO-d₆): δ 11.15 (s, 1H), 8.71 (s, 1H), 8.28-8.26 (m, 2H), 8.00 (s, 1H), 7.43 (s, 1H), 5.54 (d, J=5.2 Hz, 1H), 4.78-4.73 (m, 1H), 3.69-3.65 (m, 1H), 3.60 (s, 3H), 3.56-3.51 (m, 1H), 3.32 (s, 3H), 2.21 (s, 3H), 2.13-2.06 (m, 1H), 0.91-0.84 (m, 4H).

The absolute stereochemistry of enantiomers A and B was not assigned. The two enantiomeric structures that could be obtained from chiral separation of the enantiomeric mixture as described above are shown as Compounds 146 and 147 in Table 1.

### Example S61. Synthesis of (1R,2R)-2-fluoro-N-(1-methyl-3-(4-methyl-6-propionylpyridin-3-yl)-2-oxo-1,2-dihydro-1,6-naphthyridin-7-yl)cyclopropane-1-carboxamide (Compound 148)

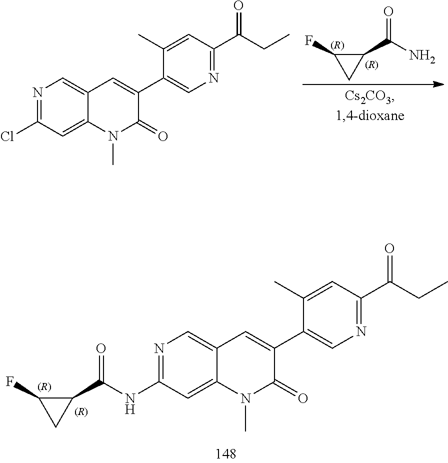

To a solution of 7-chloro-1-methyl-3-(4-methyl-6-propionylpyridin-3-yl)-1,6-naphthyridin-2(1H)-one (200.0 mg, 0.59 mmol) in dioxane (5.0 mL) was added (1R,2R)-2-fluorocyclopropane-1-carboxamide (120.6 mg, 1.17 mmol), Cs₂CO₃ (571.9 mg, 1.75 mmol), Pd(OAc)₂ (13.1 mg, 0.06 mmol) and XPhos (55.8 mg, 0.12 mmol) at room temperature under N₂. The resulting mixture was stirred at 80° C. for 4 h. After the reaction was completed, the reaction mixture was diluted with water and extracted with ethyl acetate. The combined organic layer was washed with brine, dried over anhydrous Na₂SO₄ and filtered. The filtrate was concentrated under reduced pressure. The residue was purified by flash column chromatography with petroleum ether/ethyl acetate (30/70, v/v) and then purified by Prep-HPLC with the following conditions: (Column: Sunfire prep C18 column, 30×150 mm, 5 m; Mobile Phase A: ACN, Mobile Phase B: Water (0.1% FA); Flow rate: 60 mL/min; Gradient: 32% B to 35% B in 9 min; Wave Length: 254/220 nm) to afford (1R,2R)-2-fluoro-N-(1-methyl-3-(4-methyl-6-propionylpyridin-3-yl)-2-oxo-1,2-dihydro-1,6-naphthyridin-7-yl)cyclopropane-1-carboxamide (Compound 148) (24.4 mg, 10%) as a white solid. LCMS (ESI, m/z): [M+H]⁺=409.2. ¹H NMR (400 MHz, DMSO-d₆): δ 11.22 (s, 1H), 8.74 (s, 1H), 8.53 (s, 1H), 8.27 (s, 1H), 8.09 (s, 1H), 7.91 (s, 1H), 5.07-4.88 (m, 1H), 3.63 (s, 3H), 3.32-3.18 (m, 2H), 2.34-2.27 (m, 4H), 1.76-1.69 (m, 1H), 1.28-1.20 (m, 1H), 1.15-1.11 (m, 3H).

### Example S67. Synthesis of N-(3-{6-[(1S)-1-hydroxybutyl]-4-methylpyridin-3-yl}-1-methyl-2-oxo-1,6-naphthyridin-7-yl)acetamide and N-(3-{6-[(1R)-1-hydroxybutyl]-4-methylpyridin-3-yl}-1-methyl-2-oxo-1,6-naphthyridin-7-yl)acetamide (Compound 149 and 150)

### Step 1. Synthesis of N-[3-(6-butanoyl-4-methylpyridin-3-yl)-1-methyl-2-oxo-1,6-naphthyridin-7-yl]acetamide

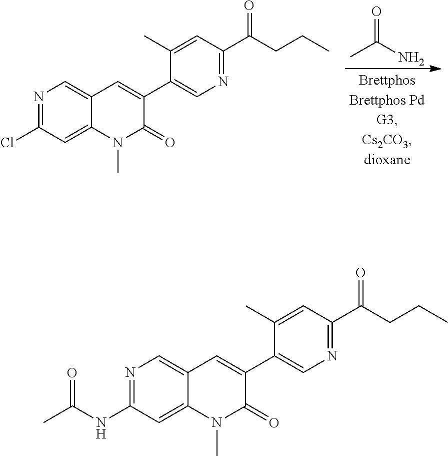

A mixture of 3-(6-butanoyl-4-methylpyridin-3-yl)-7-chloro-1-methyl-1,6-naphthyridin-2-one (1.3 g, 3.65 mmol), acetamide (0.6 g, 10.96 mmol), Cs₂CO₃ (3.5 g, 10.96 mmol), BrettPhos (0.3 g, 0.73 mmol) and BrettPhos Pd G3 (0.3 g, 0.36 mmol) in dioxane (20.0 mL) was stirred at 100° C. for 16 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (94/6, v/v) to afford N-[3-(6-butanoyl-4-methylpyridin-3-yl)-1-methyl-2-oxo-1,6-naphthyridin-7-yl]acetamide (420.0 mg, 30%) as a yellow solid. LCMS (ESI, m/z): [M+H]⁺=379.2.

### Step 2. Synthesis of N-{3-[6-(1-hydroxybutyl)-4-methylpyridin-3-yl]-1-methyl-2-oxo-1,6-naphthyridin-7-yl}acetamide

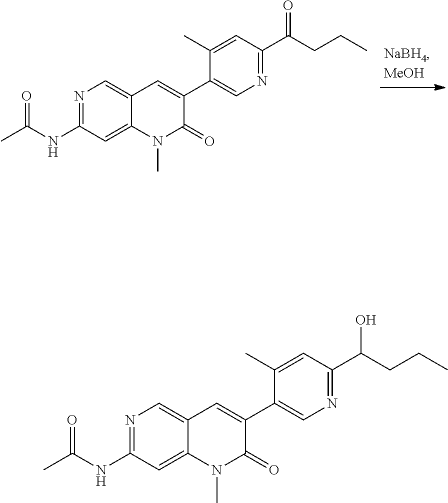

To a stirred solution of N-[3-(6-butanoyl-4-methylpyridin-3-yl)-1-methyl-2-oxo-1,6-naphthyridin-7-yl]acetamide (400.0 mg, 1.05 mmol) in THE (10.0 mL) and CH₃OH (1.0 mL) was added NaBH₄ (179.9 mg, 4.75 mmol) at 0° C. under N₂. The resulting mixture was stirred at room temperature for 4 h. After the reaction was completed, the reaction mixture was quenched with water and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (94/6, v/v) to afford N-{3-[6-(1-hydroxybutyl)-4-methylpyridin-3-yl]-1-methyl-2-oxo-1,6-naphthyridin-7-yl}acetamide (80.0 mg, 19%) as a yellow solid. LCMS (ESI, m/z): [M+H]⁺=381.2.

### Step 3. Chiral Separation of N-(3-{6-[(1S)-1-hydroxybutyl]-4-methylpyridin-3-yl}-1-methyl-2-oxo-1,6-naphthyridin-7-yl)acetamide and N-(3-{6-[(1R)-1-hydroxybutyl]-4-methylpyridin-3-yl}-1-methyl-2-oxo-1,6-naphthyridin-7-yl)acetamide (Compound 149 and 150)

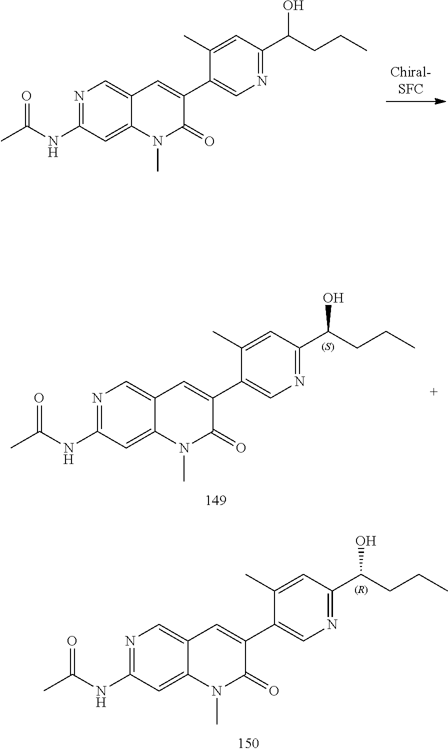

The racemic product of N-{3-[6-(1-hydroxybutyl)-4-methylpyridin-3-yl]-1-methyl-2-oxo-1,6-naphthyridin-7-yl}acetamide (80.0 mg, 0.21 mmol) was separated by Prep-Chiral-HPLC with the following conditions (Column: CHIRALPAK ID, 2×25 cm, 5 m; Mobile Phase A: Hex (0.2% IPAmine)-HPLC, Mobile Phase B: EtOH:DCM=1:1-HPLC; Flow rate: 20 mL/min; Gradient: 40% B to 40% B in 18.5 min; Wave Length: 220/254 nm; RT1(min): 12.43; RT2(min): 14.48) to afford N-(3-{6-[1-hydroxybutyl]-4-methylpyridin-3-yl}-1-methyl-2-oxo-1,6-naphthyridin-7-yl)acetamide Enantiomer A (retention time 12.43 min, 35.1 mg, 87%) as an off-white solid and N-(3-{6-[1-hydroxybutyl]-4-methylpyridin-3-yl}-1-methyl-2-oxo-1,6-naphthyridin-7-yl)acetamide Enantiomer B (retention time 14.48 min, 20.9 mg, 52%) as an off-white solid.

Enantiomer A: retention time 1: 12.43 min, LCMS (ESI, m/z): [M+H]⁺=381.0. ¹H NMR (400 MHz, DMSO-d₆): δ 10.82 (s, 1H), 8.70 (s, 1H), 8.26-8.21 (s, 2H), 8.00 (s, 1H), 7.40 (s, 1H), 5.27 (d, J=5.2 Hz, 1H), 4.60-4.56 (m, 1H), 3.61 (s, 3H), 2.20-2.17 (m, 6H), 1.79-1.60 (m, 2H), 1.42-1.36 (m, 2H), 0.93-0.89 (m, 3H).

Enantiomer B: retention time 2: 14.48 min, LCMS (ESI, m/z): [M+H]⁺=381.0. ¹H NMR (400 MHz, DMSO-d₆): δ 10.82 (s, 1H), 8.70 (s, 1H), 8.26-8.22 (m, 2H), 8.00 (s, 1H), 7.40 (s, 1H), 5.27 (d, J=4.8 Hz, 1H), 4.60-4.56 (m, 1H), 3.60 (s, 3H), 2.20-2.17 (m, 6H), 1.79-1.58 (m, 2H), 1.42-1.36 (m, 2H), 0.93-0.89 (m, 3H).

The absolute stereochemistry of enantiomers A and B was not assigned. The two enantiomeric structures that could be obtained from chiral separation of the enantiomeric mixture as described above are shown as Compounds 149 and 150 in Table 1.

### Example S63. Synthesis of (1R,2R)—N-(3-(6-butyryl-4-methylpyridin-3-yl)-1-methyl-2-oxo-1,2-dihydro-1,6-naphthyridin-7-yl)-2-fluorocyclopropane-1-carboxamide (Compound 151)

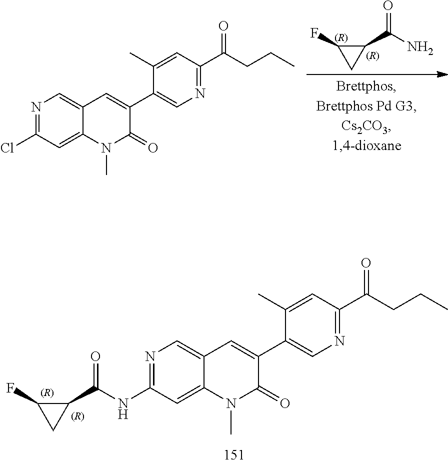

To a solution of 3-(6-butanoyl-4-methylpyridin-3-yl)-7-chloro-1-methyl-1,6-naphthyridin-2-one (80.0 mg, 0.23 mmol) in 1,4-dioxane (4.0 mL) was added BrettPhos Pd G3 (20.0 mg, 0.02 mmol), BrettPhos (20.0 mg, 0.05 mmol), (1R,2R)-2-fluorocyclopropane-1-carboxamide (35.5 mg, 0.35 mmol) and Cs₂CO₃ (219.8 mg, 0.68 mmol) at room temperature under N₂. The resulting mixture was stirred at 80° C. for 16 h under N₂. After the reaction was completed, the resulting mixture was filtered. The filtrate was concentrated under reduced pressure. The residue was purified by flash column chromatography with DCM/MeOH (92/8, v/v) and then purified by Prep-HPLC with the following conditions: (Column: XBridge Prep OBD C18 Column, 30×150 mm, 5 m; Mobile Phase A: Water (10 mmol/L NH₄HCO₃), Mobile Phase B: ACN; Flow rate: 60 mL/min; Gradient: 35% B to 40% B in 8 min; Wave Length: 254 nm) to afford (1R,2R)—N-[3-(6-butanoyl-4-methylpyridin-3-yl)-1-methyl-2-oxo-1,6-naphthyridin-7-yl]-2-fluorocyclopropane-1-carboxamide (Compound 151) (15.9 mg, 17%) as a white solid. LCMS (ESI, m/z): [M+H]⁺=423.2. ¹H NMR (400 MHz, DMSO-d₆): δ 11.22 (s, 1H), 8.75 (s, 1H), 8.53 (s, 1H), 8.27 (s, 1H), 8.09 (s, 1H), 7.91 (s, 1H), 5.08-4.88 (m, 1H), 3.63 (s, 3H), 3.19-3.15 (m, 2H), 2.34-2.27 (m, 4H), 1.76-1.64 (m, 3H), 1.29-1.22 (m, 1H), 0.98-0.90 (m, 3H).

### Example S64. Synthesis of (1S,2S)—N-(3-(6-butyryl-4-methylpyridin-3-yl)-1-methyl-2-oxo-1,2-dihydro-1,6-naphthyridin-7-yl)-2-fluorocyclopropane-1-carboxamide (Compound 152)

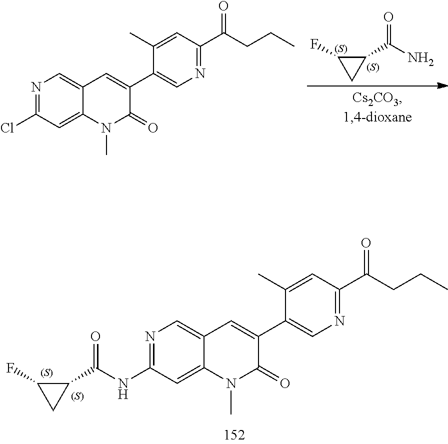

To a solution of 3-(6-butyryl-4-methylpyridin-3-yl)-7-chloro-1-methyl-1,6-naphthyridin-2(1H)-one (100.0 mg, 0.28 mmol) in 1,4-dioxane (2.0 mL) was added (1S,2S)-2-fluorocyclopropane-1-carboxamide (115.9 mg, 1.12 mmol), Cs₂CO₃ (183.1 mg, 0.56 mmol), XPhos (26.8 mg, 0.05 mmol) and Pd(OAc)₂ (6.3 mg, 0.02 mmol) at room temperature under N₂. The resulting mixture was stirred at 85° C. for 4 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl/MeOH (10/1, v/v) and then purified by Prep-HPLC with the following conditions (Column: XBridge Prep OBD C18 Column, 30×150 mm, 5 m; Mobile Phase A: Water (10 mmol/L NH₄HCO₃), Mobile Phase B: ACN; Flow rate: 60 mL/min; Gradient: 35% B to 44% B in 8 min; Wave Length: 254 nm) to afford (1S,2S)—N-(3-(6-butyryl-4-methylpyridin-3-yl)-1-methyl-2-oxo-1,2-dihydro-1,6-naphthyridin-7-yl)-2-fluorocyclopropane-1-carboxamide (Compound 152) (16.8 mg, 13%) as a white solid. LCMS (ESI, m/z): [M+H]⁺=423.2. ¹H NMR (400 MHz, DMSO-d₆): δ 11.22 (s, 1H), 8.75 (s, 1H), 8.53 (s, 1H), 8.27 (s, 1H), 8.09 (s, 1H), 7.91 (s, 1H), 5.07-4.89 (m, 1H), 3.62 (s, 3H), 3.19-3.15 (m, 2H), 2.33-2.29 (m, 4H), 1.74-1.65 (m, 3H), 1.27-1.23 (m, 1H), 0.98-0.94 (m, 3H).

### Example S65. Synthesis of N-(3-{6-[(1S)-2-cyclopropyl-1-hydroxyethyl]-4-methylpyridin-3-yl}-1-methyl-2-oxo-1,6-naphthyridin-7-yl)cyclopropanecarboxamide and N-(3-{6-[(1R)-2-cyclopropyl-1-hydroxyethyl]-4-methylpyridin-3-yl}-1-methyl-2-oxo-1,6-naphthyridin-7-yl)cyclopropanecarboxamide (Compound 157 and 158)

### Step 1. Synthesis of 1-(5-bromo-4-methylpyridin-2-yl)-2-cyclopropylethanone

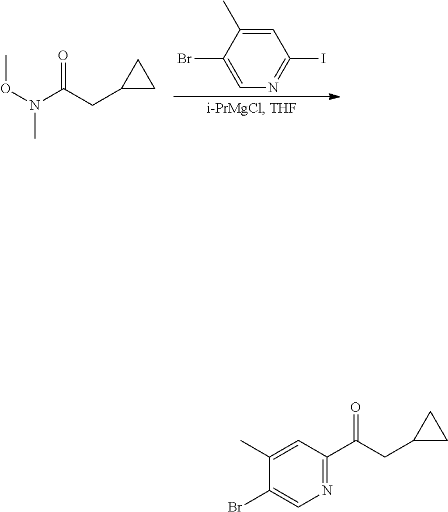

To a solution of 5-bromo-2-iodo-4-methylpyridine (6.0 g, 20.20 mmol) in THE (70.0 mL) was added dropwise i-PrMgCl (24.2 mL, 1 mol/L) at 0° C. under N₂. The resulting mixture was stirred at 0° C. for 10 min under N₂. Then a solution of 2-cyclopropyl-N-methoxy-N-methylacetamide (3.4 g, 23.9 mmol) in THE (30.0 mL) was added dropwise to the mixture at 0° C. under N₂. The resulting mixture was stirred at 0° C. for additional 2 h under N₂. After the reaction was completed, the reaction mixture was quench with H₂O at 0° C. and then extracted with ethyl acetate. The combined organic layer was washed with brine, dried over anhydrous sodium sulfate and filtered. The filtrate was concentrated under reduced pressure. The residue was purified by reverse phase flash column chromatography with petroleum CH₃CN/H₂O (60/40, v/v) to afford 1-(5-bromo-4-methylpyridin-2-yl)-2-cyclopropylethanone (1.5 g, 30%) as a yellow solid. LCMS (ESI, m/z): [M+H]⁺=254.0

### Step 2. Synthesis of 2-cyclopropyl-1-(4-methyl-5-(4,4,5,5-tetramethyl-1,3,2-dioxaborolan-2-yl)pyridin-2-yl)ethan-1-one

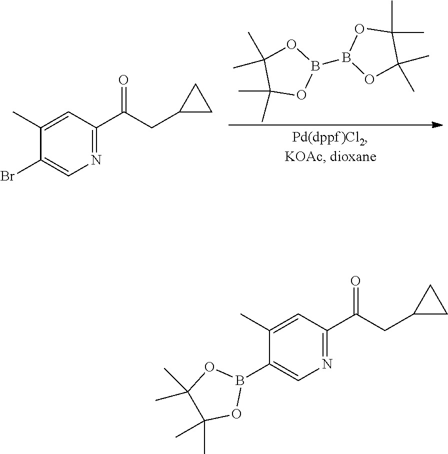

To a solution of 1-(5-bromo-4-methylpyridin-2-yl)-2-cyclopropylethanone (1.5 g, 5.93 mmol) in dioxane (60.0 mL) was added bis(pinacolato)diboron (4.5 g, 17.79 mmol), KOAc (1.7 g, 17.79 mmol) and Pd(dppf)Cl₂ (967.8 mg, 1.19 mmol) at room temperature under N₂. The resulting mixture was stirred at 100° C. for 2 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford 2-cyclopropyl-1-(4-methyl-5-(4,4,5,5-tetramethyl-1,3,2-dioxaborolan-2-yl)pyridin-2-yl)ethan-1-one (1.5 g, 84%) as a brown solid. LCMS (ESI, m/z): [M+H]⁺=302.2.

### Step 3. Synthesis of 7-chloro-3-[6-(2-cyclopropylacetyl)-4-methylpyridin-3-yl]-1-methyl-1,6-naphthyridin-2-one

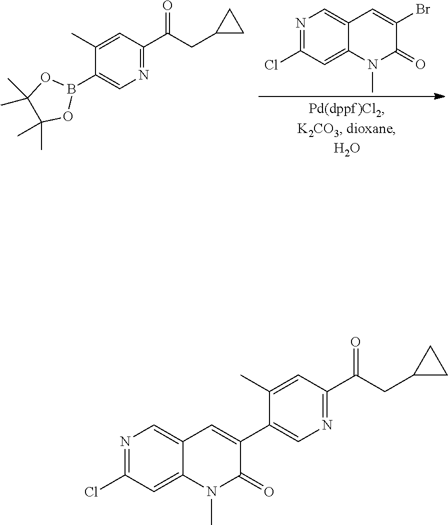 [SMILES: Cc1cc(C(=O)CC2CC2)ncc1B1OC(C)(C)C(C)(C)O1 ; Cn1c(=O)c(Br)cc2cnc(Cl)cc21 ; Cc1cc(C(=O)CC2CC2)ncc1-c1cc2cnc(Cl)cc2n(C)c1=O]

To a stirred mixture of 2-cyclopropyl-1-[4-methyl-5-(4,4,5,5-tetramethyl-1,3,2-dioxaborolan-2-yl)pyridin-2-yl]ethanone (1.4 g, 4.75 mmol) in dioxane (20.0 mL) and H₂O (2.0 mL) were added 3-bromo-7-chloro-1-methyl-1,6-naphthyridin-2-one (1.0 g, 3.66 mmol), Pd(dppf)Cl₂ (0.3 g, 0.37 mmol) and K₂CO₃ (1.5 g, 10.97 mmol) at room temperature under N₂. The resulting mixture was stirred at 80° C. for 2 h under N₂. After the reaction was completed, the mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford 7-chloro-3-[6-(2-cyclopropylacetyl)-4-methylpyridin-3-yl]-1-methyl-1,6-naphthyridin-2-one (850.0 mg, 63%) as a white solid. LCMS (ESI, m/z): [M+H]⁺=368.2.

### Step 4. Synthesis of N-{3-[6-(2-cyclopropylacetyl)-4-methylpyridin-3-yl]-1-methyl-2-oxo-1,6-naphthyridin-7-yl}cyclopropanecarboxamide

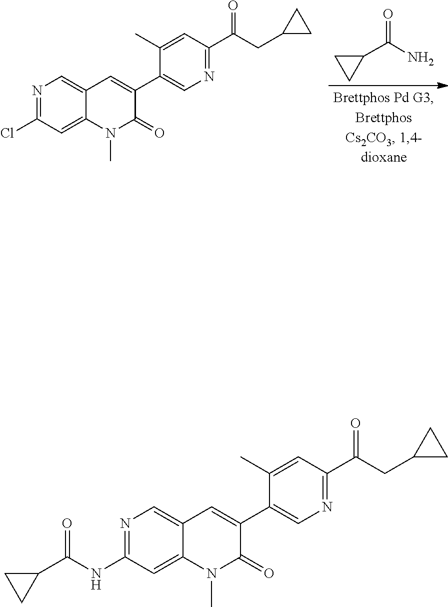

To a stirred mixture of 7-chloro-3-[6-(2-cyclopropylacetyl)-4-methylpyridin-3-yl]-1-methyl-1,6-naphthyridin-2-one (850.0 mg, 2.31 mmol) in dioxane (15.0 mL) was added cyclopropanecarboxamide (295.0 mg, 3.47 mmol), BrettPhos Pd G3 (419.0 mg, 0.46 mmol), BrettPhos (496.2 mg, 0.92 mmol) and Cs₂CO₃ (2.26 g, 6.93 mmol) at room temperature under N₂. The resulting mixture was stirred at 85° C. for 2 h under N₂. After the reaction was completed, the mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/5, v/v) to afford N-{3-[6-(2-cyclopropylacetyl)-4-methylpyridin-3-yl]-1-methyl-2-oxo-1,6-naphthyridin-7-yl}cyclopropanecarboxamide (160.0 mg, 16%) as a yellow solid. LCMS (ESI, m/z): [M+H]⁺=417.2.

### Step 5. Synthesis of N-{3-[6-(2-cyclopropyl-1-hydroxyethyl)-4-methylpyridin-3-yl]-1-methyl-2-oxo-1,6-naphthyridin-7-yl}cyclopropanecarboxamide

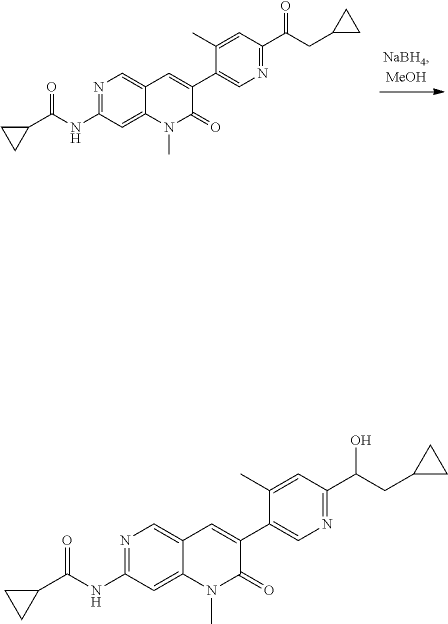 [SMILES: Cc1cc(C(=O)CC2CC2)ncc1-c1cc2cnc(NC(=O)C3CC3)cc2n(C)c1=O ; Cc1cc(C(O)CC2CC2)ncc1-c1cc2cnc(NC(=O)C3CC3)cc2n(C)c1=O]

To a stirred mixture of N-{3-[6-(2-cyclopropylacetyl)-4-methylpyridin-3-yl]-1-methyl-2-oxo-1,6-naphthyridin-7-yl}cyclopropanecarboxamide (160.0 mg, 0.38 mmol) in THE (5.0 mL) and methanol (0.5 mL) were added NaBH₄ (43.6 mg, 1.15 mmol) at 0° C. under N₂. The resulting mixture was stirred at room temperature for 2 h. After the reaction was completed, the reaction mixture was diluted with H₂O at 0° C. The resulting mixture was extracted with ethyl acetate. The combined organic layer was washed with brine, dried over anhydrous sodium sulfate and filtered. The filtrate was concentrated under reduced pressure. The residue was purified by flash column chromatography with CH₂Cl₂/CH₃OH (10/1, v/v) to afford N-{3-[6-(2-cyclopropyl-1-hydroxyethyl)-4-methylpyridin-3-yl]-1-methyl-2-oxo-1,6-naphthyridin-7-yl}cyclopropanecarboxamide (80.0 mg, 49%) as a white solid. LCMS (ESI, m/z): [M+H]⁺=419.2.

### Step 6. Chiral Separation of N-(3-{6-[(1S)-2-cyclopropyl-1-hydroxyethyl]-4-methylpyridin-3-yl}-1-methyl-2-oxo-1,6-naphthyridin-7-yl)cyclopropanecarboxamide and N-(3-{6-[(1R)-2-cyclopropyl-1-hydroxyethyl]-4-methylpyridin-3-yl}-1-methyl-2-oxo-1,6-naphthyridin-7-yl)cyclopropanecarboxamide (Compound 157 and 158)

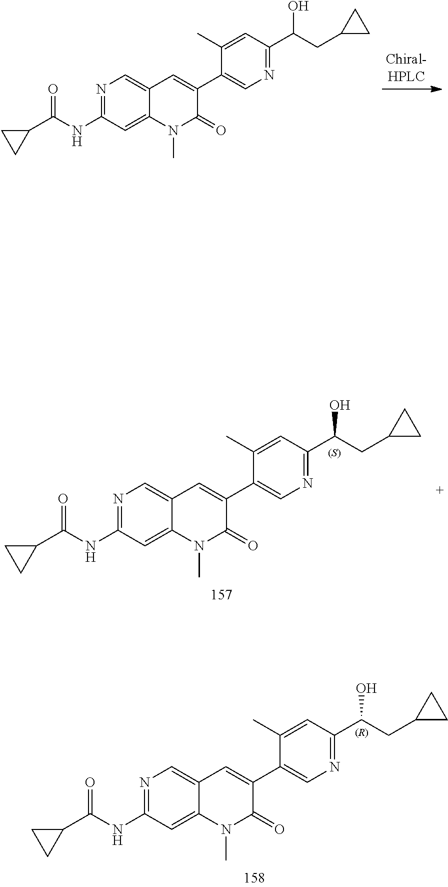

The product of N-{3-[6-(2-cyclopropyl-1-hydroxyethyl)-4-methylpyridin-3-yl]-1-methyl-2-oxo-1,6-naphthyridin-7-yl}cyclopropanecarboxamide (80.0 mg, 0.19 mmol) was separated by Prep-Chiral-HPLC with the following conditions (Column: CHIRAL ART Cellulose-SC, 2×25 cm, 5 m; Mobile Phase A: Hex (0.5% 2M NH₃-MeOH)-HPLC, Mobile Phase B: EtOH:DCM=1:1-HPLC; Flow rate: 20 mL/min; Gradient: 60% B to 60% B in 16 min; Wave Length: 254/220 nm; RT1(min): 10.05; RT2(min): 13.35) to afford N-(3-{6-[2-cyclopropyl-1-hydroxyethyl]-4-methylpyridin-3-yl}-1-methyl-2-oxo-1,6-naphthyridin-7-yl)cyclopropanecarboxamide Enantiomer A (retention time 10.05 min, 27.6 mg, 69%) as a white solid and N-(3-{6-[2-cyclopropyl-1-hydroxyethyl]-4-methylpyridin-3-yl}-1-methyl-2-oxo-1,6-naphthyridin-7-yl)cyclopropanecarboxamide Enantiomer B (retention time 13.35 min, 13.3 mg, 33%) as a white solid.

Enantiomer A: retention time 1: 10.05 min; LCMS (ESI, m/z): [M+H]⁺=419.1. ¹H NMR (400 MHz, DMSO-d₆): δ 11.15 (s, 1H), 8.71 (s, 1H), 8.25 (s, 2H), 7.99 (s, 1H), 7.42 (s, 1H), 5.36 (d, J=5.2 Hz, 1H), 4.66-4.62 (m, 1H), 3.59 (s, 3H), 2.20 (s, 3H), 2.12-2.06 (m, 1H), 1.74-1.67 (m, 1H), 1.54-1.47 (m, 1H), 0.89-0.82 (m, 5H), 0.42-0.37 (m, 2H), 0.07-0.01 (m, 2H).

Enantiomer B: retention time 2: 13.35 min; LCMS (ESI, m/z): [M+H]⁺=419.1. ¹H NMR (400 MHz, DMSO-d₆): δ 11.15 (s, 1H), 8.71 (s, 1H), 8.25 (s, 2H), 7.99 (s, 1H), 7.42 (s, 1H), 5.34 (d, J=4.4 Hz, 1H), 4.66-4.62 (m, 1H), 3.59 (s, 3H), 2.20 (s, 3H), 2.12-2.06 (m, 1H), 1.74-1.67 (m, 1H), 1.56-1.47 (m, 1H), 0.90-0.84 (m, 5H), 0.44-0.36 (m, 2H), 0.09-0.01 (m, 2H).

The absolute stereochemistry of enantiomers A and B was not assigned. The two enantiomeric structures that could be obtained from chiral separation of the enantiomeric mixture as described above are shown as Compounds 157 and 158 in Table 1.

### BIOLOGICAL EXAMPLES

### Example B1.K562 Cell Proliferation Assay

K562 cells, cultured in Iscove's Modified Dulbecco's Media (IMDM) supplemented with 10% FBS, were harvested at 50-80% confluency and plated at 2,000 cells per well in 384-well tissue culture plates. A subset of wells contained media only (low control, LC). Compounds were serially diluted in DMSO. 40 nL of compound or DMSO only (high control, HC) were added to each well using an Echo 550 liquid handler (Labcyte). The plates were placed in a 37° C. incubator with 5% CO₂ for 72 hours. Cell viability was measured using a CellTiter-Glo luminescent cell viability assay (Promega), which allows for relative quantification of metabolically active cells by luminescence-based measurements of intracellular ATP concentrations. Briefly, plates were removed from the incubator and equilibrated for 15 minutes at room temperature prior to the addition of 40 μL of CellTiter-Glo reagent. Plates were then incubated for 30 minutes at room temperature. Luminescence was measured using an EnSpire plate reader (Perkin Elmer). As noted above, luminescence values from wells with DMSO only or media without cells were used as high and low controls (HC and LC), respectively. Normalized percent viability was calculated as follows: percent viability=100×(Lum_{Sample}−Lum_{LC})/(Lum_{HC}−Lum_{LC}). IC₅₀ values were calculated using the XLFit software.

TABLE 2

| Cell Line | IC₅₀ (nM) Compound 2 | IC₅₀ (nM) Compound 28 |
|---|---|---|
| K562 | 28 | 47 |

### Example B2. Radioactivity-Based ABL Kinase Assay

The following kinase assay was based on Anastassiadis et al (2011) Nat Biotech, 29:1039-1045. Briefly, ABL kinase substrate (ABLtide: EAIYAAPFAKKK; Genscript) was freshly prepared in reaction buffer (20 mM HEPES pH 7.5, 10 mM MgCl₂, 1 mM EGTA, 0.02% Brij-35, 0.02 mg/ml BSA, 0.1 mM Na₃VO₄, 2 mM DTT, and 1% DMSO) to a final substrate concentration of 20 μM. ABL wild type and ABL mutant kinases were added to the substrate solution and mixed to the final enzyme concentrations shown in Table 3 below.

TABLE 3

Enzyme Conc (nM)

| | Compound 2 | Compound 28 |
|---|---|---|
| ABL1 | 0.2 | 0.2 |
| ABL1 (T315I) | 4 | 5 |
| ABL1 (E255K) | 1 | 1.5 |
| ABL1 (E255V) | 0.05 | 0.1 |
| ABL1 (F317I) | 0.5 | 8 |
| ABL1 (F317L) | 1.2 | 1 |
| ABL1 (G250E) | 1.25 | 1.75 |
| ABL1 (H396P) | 3 | 1.5 |
| ABL1 (M351T) | 4 | 4 |
| ABL1 (Q252H) | 1.5 | 0.75 |
| ABL1 (V299L) | 0.2 | 0.35 |
| ABL1 (Y253F) | 0.3 | 0.2 |
| ABL1 (Y253H) | 0.15 | 0.5 |

Serial dilutions of compounds were prepared in 100% DMSO Compounds or DMSO only (vehicle control) were then added to the kinase reaction mixture and incubated for 20 minutes at room temperature. 100 μM ³³P-ATP (specific activity 10 μCi/μl) was then added to the kinase reaction mixture to initiate the kinase reaction. This mixture was incubated for 2 hours at room temperature. Radioactivity was then measured by radioisotope filter-binding. Kinase activity data were expressed as the percent remaining kinase activity in test samples compared to vehicle control reactions. IC₅₀ values and curve fits were obtained using a nonlinear regression model with a sigmoidal dose response (Prism GraphPad Software).

### Example B3. Luminescence-Based ABL Kinase Assay (Km ATP)

Kinase activity of ABL1 was measured using the ADP-Glo system (Promega), which measures formation of ADP using a luminescence-based method. Compounds were serially diluted in DMSO. 20 nL of compound or DMSO only (high control, HC) were added to a 384-well plate (OptiPlate-384, PerkinElmer) using an Echo550 liquid handler (Labcyte). 15 μL of kinase solution (10 mM MgCl₂, 0.01% Brij-35, 2 mM DTT, 0.05% BSA, 1 mM EGTA, 50 mM HEPES pH 7.5, and 3.325 nM ABL1 [Carna Biosciences]) were added to each well of the 384-well plate containing the compounds. No enzyme control wells were included (low control, LC). The plate was incubated at room temperature for 30 minutes. 5 μL of a second solution containing 10 mM MgCl₂, 0.01% Brij-35, 2 mM DTT, 0.05% BSA, 1 mM EGTA, 50 mM HEPES pH 7.5, 6 μM Peptide 2 (Perkin Elmer, Cat No 760346), and 40 μM ATP were added to each well to start the kinase reaction. The plate was incubated for 90 minutes at room temperature. 20 μL of ADP-Glo reagent (Promega) were then added to each well and the plate was incubated for 40 minutes at room temperature. 40 μL of kinase detection reagent (Promega) was added to each well and the plate was incubated for an additional 45 minutes at room temperature. During this step, ADP was converted to ATP, a substrate for luciferase, to produce luminescence signal. Luminescence was measured on an Envision plate reader (Perkin Elmer). Luminescence signal positively correlates with kinase activity. The percent kinase activity was calculated as follows: percent kinase activity=100×(Lum_{Sample}−Lum_{LC})/(Lum_{HC}−Lum_{LC}). As noted above, DMSO only and no enzyme wells were used as high and low controls, respectively. IC₅₀ values were calculated using the XLFit software.

IC₅₀ data obtained using the screening procedures described above for certain compounds disclosed herein are listed in Table 4.

TABLE 4

| Kinase | IC₅₀ (nM) Compound 2 | IC₅₀ (nM) Compound 28 |
|---|---|---|
| ABL1 | 0.61 (1.8) | 1.2 (2.2) |
| ABL1 (T315I) | 4400 (680) | >500 (1500) |
| ABL1 (E255K) | 2.0 | 3.4 |
| ABL1 (E255V) | 0.61 | 1.4 |
| ABL1 (F317I) | 47 | 114 |
| ABL1 (F317L) | 17 | 107 |
| ABL1 (G250E) | 1.5 | 3.7 |
| ABL1 (H396P) | 1.2 | 1.4 |
| ABL1 (M351T) | 1.5 | 4.5 |
| ABL1 (Q252H) | 1.2 | 1.7 |
| ABL1 (V299L) | <0.51 | 0.19 |
| ABL1 (Y253F) | <0.51 | 0.53 |
| ABL1 (Y253H) | 0.93 | 3.6 |

IC₅₀ values inside parentheses were measured using the Abl1 Km assay

In addition to evaluating their inhibition activity against Abl1 kinase mutants, a selection of compounds were further assayed for their inhibition of off-target kinase activity, including c-KIT, KDR (VEGFR2), PDGFRα, and SRC kinases.

### Example B4. Luminescence-Based c-KIT Kinase Assay (Km ATP)

Kinase activity of c-KIT was measured using the ADP-Glo system (Promega). Compounds were diluted in DMSO at 100 μM. 20 nL of compound or DMSO only (high control, HC) were added to a 384-well plate (OptiPlate-384, PerkinElmer) using an Echo550 liquid handler (Labcyte). 15 μL of kinase solution (10 mM MgCl₂, 0.01% Brij-35, 2 mM DTT, 0.05% BSA, 1 mM EGTA, 50 mM HEPES pH 7.5, and 13.3 nM c-KIT [Carna Biosciences]) were added to each well of the 384-well plate containing the compounds. No enzyme control wells were included (low control, LC). The plate was incubated at room temperature for 30 minutes. 5 μL of a second solution containing 10 mM MgCl₂, 0.01% Brij-35, 2 mM DTT, 0.05% BSA, 1 mM EGTA, 50 mM HEPES pH 7.5, 0.8 μg/ml Poly(Glu-Tyr) (Sigma, Cat No 97105-00-5), and 400 μM ATP were added to each well to start the kinase reaction. The plate was incubated for 90 minutes at room temperature. 20 μL of ADP-Glo reagent (Promega) were then added to each well and the plate was incubated for 40 minutes at room temperature. 40 μL of kinase detection reagent (Promega) was added to each well and the plate was incubated for an additional 45 minutes at room temperature. During this step, ADP was converted to ATP, a substrate for luciferase, to produce luminescence signal. Luminescence was measured on an Envision plate reader (Perkin Elmer). Luminescence signal positively correlates with kinase activity. The percent inhibition of kinase activity was calculated as follows: percent inhibition=100×(Lum_{HC}−Lum_{Sample})/(Lum_{HC}−Lum_{LC}). As noted above, DMSO only and no enzyme wells were used as high and low controls, respectively.

### Example B5. Luminescence-Based KDR Kinase Assay (Km ATP)

Kinase activity of KDR was measured using the ADP-Glo system (Promega). Compounds were diluted in DMSO at 100 μM. 20 nL of compound or DMSO only (high control, HC) were added to a 384-well plate (OptiPlate-384, PerkinElmer) using an Echo550 liquid handler (Labcyte). 15 μL of kinase solution (10 mM MgCl₂, 0.01% Brij-35, 2 mM DTT, 0.05% BSA, 1 mM EGTA, 0.05% CHAPSO, 50 mM HEPES pH 7.5, and 0.665 nM KDR [Carna Biosciences]) were added to each well of the 384-well plate containing the compounds. No enzyme control wells were included (low control, LC). The plate was incubated at room temperature for 30 minutes. 5 μL of a second solution containing 10 mM MgCl₂, 0.01% Brij-35, 2 mM DTT, 0.05% BSA, 1 mM EGTA, 50 mM HEPES pH 7.5, 6 μM Peptide 22 (Perkin Elmer, Cat No 760366), and 400 μM ATP were added to each well to start the kinase reaction. The plate was incubated for 90 minutes at room temperature. 20 μL of ADP-Glo reagent (Promega) were then added to each well and the plate was incubated for 40 minutes at room temperature. 40 μL of kinase detection reagent (Promega) was added to each well and the plate was incubated for an additional 45 minutes at room temperature. During this step, ADP was converted to ATP, a substrate for luciferase, to produce luminescence signal. Luminescence was measured on an Envision plate reader (Perkin Elmer). Luminescence signal positively correlates with kinase activity. The percent inhibition of kinase activity was calculated as follows: percent inhibition=100×(Lum_{HC}−Lum_{Sample})/(Lum_{HC}−Lum_{LC}). As noted above, DMSO only and no enzyme wells were used as high and low controls, respectively.

### Example B6. Luminescence-Based PDGFRα Kinase Assay (Km ATP)

Kinase activity of PDGFRα was measured using the ADP-Glo system (Promega). Compounds were diluted in DMSO at 100 μM. 20 nL of compound or DMSO only (high control, HC) were added to a 384-well plate (OptiPlate-384, PerkinElmer) using an Echo550 liquid handler (Labcyte). 15 μL of kinase solution (10 mM MgCl₂, 0.01% Brij-35, 2 mM DTT, 0.05% BSA, 1 mM EGTA, 50 mM HEPES pH 7.5, and 13.3 nM PDGFRα [Carna Biosciences]) were added to each well of the 384-well plate containing the compounds. No enzyme control wells were included (low control, LC). The plate was incubated at room temperature for 30 minutes. 5 μL of a second solution containing 10 mM MgCl₂, 0.01% Brij-35, 2 mM DTT, 0.05% BSA, 1 mM EGTA, 50 mM HEPES pH 7.5, 0.8 μg/ml Poly(Glu-Tyr) (Sigma, Cat No 97105-00-5), and 120 μM ATP were added to each well to start the kinase reaction. The plate was incubated for 90 minutes at room temperature. 20 μL of ADP-Glo reagent (Promega) were then added to each well and the plate was incubated for 40 minutes at room temperature. 40 μL of kinase detection reagent (Promega) was added to each well and the plate was incubated for an additional 45 minutes at room temperature. During this step, ADP was converted to ATP, a substrate for luciferase, to produce luminescence signal. Luminescence was measured on an Envision plate reader (Perkin Elmer). Luminescence signal positively correlates with kinase activity. The percent inhibition of kinase activity was calculated as follows: percent inhibition=100×(Lum_{HC}−Lum_{Sample})/(Lum_{HC}−Lum_{LC}). As noted above, DMSO only and no enzyme wells were used as high and low controls, respectively.

### Example B7. Luminescence-Based SRC Kinase Assay (Km ATP)

Kinase activity of SRC was measured using the ADP-Glo system (Promega). Compounds were diluted in DMSO at 100 μM. 20 nL of compound or DMSO only (high control, HC) were added to a 384-well plate (OptiPlate-384, PerkinElmer) using an Echo550 liquid handler (Labcyte). 15 μL of kinase solution (10 mM MgCl₂, 0.01% Brij-35, 2 mM DTT, 0.05% BSA, 1 mM EGTA, 50 mM HEPES pH 7.5, and 0.532 nM SRC [Carna Biosciences]) were added to each well of the 384-well plate containing the compounds. No enzyme control wells were included (low control, LC). The plate was incubated at room temperature for 30 minutes. 5 μL of a second solution containing 10 mM MgCl₂, 0.01% Brij-35, 2 mM DTT, 0.05% BSA, 1 mM EGTA, 50 mM HEPES pH 7.5, 6 μM Peptide 4 (Perkin Elmer, Cat No 760348), and 200 μM ATP were added to each well to start the kinase reaction. The plate was incubated for 90 minutes at room temperature. 20 μL of ADP-Glo reagent (Promega) were then added to each well and the plate was incubated for 40 minutes at room temperature. 40 μL of kinase detection reagent (Promega) was added to each well and the plate was incubated for an additional 45 minutes at room temperature. During this step, ADP was converted to ATP, a substrate for luciferase, to produce luminescence signal. Luminescence was measured on an Envision plate reader (Perkin Elmer). Luminescence signal positively correlates with kinase activity. The percent inhibition of kinase activity was calculated as follows: percent inhibition=100×(Lum_{HC}−Lum_{Sample})/(Lum_{HC}−Lum_{LC}). As noted above, DMSO only and no enzyme wells were used as high and low controls, respectively.

Percent inhibition of key off-target kinases using the screening procedures described above for certain compounds at 100 nM concentration disclosed herein are listed in Table 5.

TABLE 5

| | % Inhibition | % Inhibition |
|---|---|---|
| Kinase | Compound 2 | Compound 28 |
| C-Kit | 17 | 7.3 |
| KDR (VEGFR2) | 32 | 27 |
| PDGFRα | 33 | 15 |
| SRC | 77 | 48 |

### Example B8. Cell Viability Assays

Cell viability was measured in the following MAPK pathway mutant cancer cell lines: A375 (BRAF V600E), HepG2 (NRAS Q61L), SK-MEL-30 (NRAS Q61K), and OCI-AML-2 (MBNL1-CRAF fusion). Cell viability was also measured in K562 cells.

A375, HepG2, SK-MEL-30 and OCI-AML-2 cells were grown in the appropriate growth medium as described in Table B2 below, and harvested at 50-80% confluence. Cells were counted and seeded at their appropriate density (see Table B1) in a 384-well plate (Corning 3570). A375, HepG2 and SK-MEL-30 were allowed to adhere overnight prior to treatment and the OCI-AML-2 were treated immediately for the indicated drug treatment times (Table 6).

Table 6 provides the growth media, number of cells seeded per well and drug treatment times for the each cell line. K562 cells were grown as described in Example B1 above.

TABLE 6

| | | Number of | Treatment |
|---|---|---|---|
| | | cells seeded | time |
| Cell Line | Growth Medium | per well | (hours) |
| A375 | Dulbecco's Modified | 1000 | 72 |
| | Eagle Medium (DMEM) + | | |
| | 10% fetal bovine | | |
| | serum (FBS) | | |
| A375 | Dulbecco's Modified | 250 | 72 |
| | Eagle Medium (DMEM) + | | |
| | 10% fetal bovine | | |
| | serum (FBS) | | |
| HepG2 | DMEM + 10% FBS | 750 | 120 |
| SK-MEL-30 | DMEM + 10% FBS | 750 | 120 |
| OCI-AML-2 | Alpha-MEM with | 1000 | 72 |
| | nucleosides + 10% FBS | | |

Compounds were dissolved in DMSO and serially diluted. Serially-diluted compound or a DMSO only control (high control, “HC”) was added to the plated cells in each well. Compounds were tested at concentrations of about 10 μM to 0.51 nM, using three-fold dilutions. The final proportion of DMSO never exceeded 0.1%.

Plates were placed in a 37° C., 500 CO₂ incubator for the indicated treatment times (Table B1). Plates were then removed from the incubator and equilibrated for 15 minutes at room temperature. 40 μl of CellTiter Glo reagent (Promega) was added to measure the relative level of metabolically active cells by quantifying intracellular ATP concentrations. Plates were incubated for 30 minutes at room temperature, and luminescence was measured. Percent viability was normalized to a vehicle control only using the following formula: 0 viability=100×(Lum_{Sample}−Lum_{LC})/(Lum_{HC}−Lum_{LC}). IC₅₀ values were calculated using XLFit software or Prism (GraphPad Software), as shown in Table 7, below. Graphical curves were fitted using a nonlinear regression model with a sigmoidal dose response.

TABLE 7

| | BRAF Cytotoxicity (IC₅₀, nM) | | | | | |
|---|---|---|---|---|---|---|
| | Cell Line | | | | | |
| | A375 | A375 | | SK- | OCI- | |
| Compound | (1000 cells) | (250 cells) | HEPG2 | MEL-30 | AML-2 | K562 |
| 2 | | | 334 | | | |
| 28 | | | 496 | | | |
| 68 | 871 | | | | 2510 | |
| 69 | 354 | 372 | 107 | 517 | 485 | 496 |
| Example S40, | 492 | | 211 | 446 | 496 | 1230 |
| Isomer A | | | | | | |
| Example S40, | 940 | | | | | |
| Isomer B | | | | | | |
| 108 | 308 | 422 | 88.7 | 311 | 309 | 1980 |
| 109 | 1630 | | | | | |
| Example S42, | 930 | | | | | |
| Enantiomer A | | | | | | |
| Example S42, | 141 | | | | | |
| Enantiomer B | | | | | | |
| Example S43, | 52.1 | 152 | 82.1 | 399 | 457 | 1690 |
| Isomer A | | | | | | |
| Example S43, | 189 | 335 | 843 | >6.15E+03 | 2710 | |
| Isomer B | | | | | | |
| Example S49, | | | 2750 | >10.0E+03 | >10.0E+03 | |
| Isomer A | | | | | | |
| Example S49, | | 74.9 | 12.6 | 103 | 153 | 733 |
| Isomer B | | | | | | |
| Example S59, | | | | | | >10.0E+03 |
| Enantiomer A | | | | | | |
| Example S59, | | | | | | |
| Enantiomer B | | | | | | |
| Example S60, | | 5950 | >10.0E+03 | >10.0E+03 | | |
| Enantiomer A | | | | | | |
| Example S60, | | 1070 | 225 | 3260 | | |
| Enantiomer B | | | | | | |
| 148 | | | | | | 2640 |
| Example S67, | | 215 | 252 | 974 | 1390 | 9480 |
| Enantiomer A | | | | | | |
| 151 | | | 37.3 | 244 | 448 | |
| 152 | | 116 | 257 | 585 | 412 | 534 |

### Example B2. Detection of Phosphorylated ERK (pERK)

A375 cells were counted and seeded at 10,000 cells/well in 384 well plates (Corning 3764) and allowed to adhere overnight.

Compounds were dissolved and serially diluted in DMSO. The compounds were then added, mixed, and incubated for four hours at 37° C., 5% CO₂. Compounds were added using four-fold dilutions at final concentrations ranging from 10 μM to 0.01 nM. DMSO only and 10 uM staurosporine were added as high and low controls.

Following the four-hour incubation with compounds, cell lysates were prepared and AlphaLISA assay measuring phosphorylated ERK was performed. Media was removed using the Apricot Designs pipettor. Lysis buffer was made from 1× AlphaLISA SureFire Assay Kit (AlphaLISA SureFire Ultra pERK 12 (Thr202/Tyr204) ALSU-PERK-A50K) lysis buffer with protease and phosphatase inhibitors. Cells were lysed by adding 10 uL to all the wells and mixed for 40 minutes on a plate shaker. 10 uL cell lysate was transferred to a new Optiplate (PerkinElmer 6007290) and incubated with 5 uL 1× acceptor mix for 2 hours in the dark. 5 uL of 1× donor mix was added to all wells and mixed by shaking followed by overnight incubation in the dark.

pERK AlphaLISA signal was read on the Envision using standard AlphaLISA settings. Percent inhibition of ERK phosphorylation was calculated by % Inhibition=100×(Lum_{HC}−Lum_{Sample})/(Lum_{HC}−Lum_{LC}). The low and high controls (LC/HC) are generated from lysate from wells with cells treated with DMSO or 10 mM Staurosporine (BioAustralis, cat #BIA-S1086), respectively. IC₅₀ values were calculated by fitting the Curve using XLfit (v5.3.1.3), equation 201: Y=Bottom+(Top−Bottom)/(1+10\hat{ }((Log IC₅₀−X)*HillSlope)). The IC₅₀ values are shown in Table 8 below.

TABLE 8

| Compound | PERK ELISA (IC₅₀, nM) |
|---|---|
| 2 | 189 |
| 28 | 146 |
| 68 | 249 |
| 69 | 43.1 |
| Example S40, Isomer A | 47.4 |
| Example S40, Isomer B | 230 |
| 108 | 17.4 |
| 109 | 76.6 |
| Example S42, Enantiomer A | 55.1 |
| Example S42, Enantiomer B | 14.7 |
| Example S43, Isomer A | 8.01 |
| Example S43, Isomer B | 14.7 |
| 116 | 78.1 |
| 117 | 61.3 |
| 118 | 1250 |
| 119 | 313 |
| 120 | >10.0E+03 |
| 121 | 151 |
| 122 | 478 |
| 123 | 119 |
| 124 | 63.5 |
| Example S49, Isomer A | 73.8 |
| Example S49, Isomer B | 8.64 |
| Example S59, Enantiomer A | 78.5 |
| Example S59, Enantiomer B | 373 |
| Example S60, Enantiomer A | 120 |
| Example S60, Enantiomer B | 19 |
| 148 | 19.3 |
| Example S67, Enantiomer A | 7.23 |
| Example S67, Enantiomer B | 23.9 |
| 151 | 10.4 |
| 152 | 8.99 |

All publications, including patents, patent applications, and scientific articles, mentioned in this specification are herein incorporated by reference in their entirety for all purposes to the same extent as if each individual publication, including patent, patent application, or scientific article, were specifically and individually indicated to be incorporated by reference.

Although the foregoing invention has been described in some detail by way of illustration and example for purposes of clarity of understanding, it is apparent to those skilled in the art that certain minor changes and modifications will be practiced in light of the above teaching. Therefore, the description and examples should not be construed as limiting the scope of the invention.

1. A compound of formula (I) 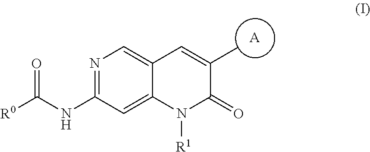
or a pharmaceutically acceptable salt, solvate, hydrate, or co-crystal thereof, or a mixture of any of the foregoing, wherein:
R⁰ is C₁-C₃ alkyl or cyclopropyl, wherein the C₁-C₃ alkyl or cyclopropyl are optionally substituted with 1 to 5 R⁵ groups;
R¹ is H, C₁-C₆ alkyl, C₁-C₆ haloalkyl, C₁-C₆ heteroalkyl, C₃-C₇ cycloalkyl, C₁-C₆ alkyl-CN, 4- to 7-membered heterocycloalkyl, —(X^{a})₀₋₁—(C(R²)R²)₀₋₁—OR²,
—(X^{a})₀₋₁—(C(R²)R²)₀₋₁—N(R²)R², —(X^{a})₀₋₁—(C(R²)R²)₀₋₁—S(O)₂R²,
—(X^{a})—(C(R²)R²)₀₋₁—S(O)(═NR²)R², —(X^{a})—C(R²)R²—S(O)₂N(R²)R²,
—(X^{a})—C(R²)R²—S(O)R², —(X^{a})—C(R²)R²—N(R²)R²S(O)₂R², or
—(X^{a})—(C(R²)R²)₀₋₁—C(O)N(R²)R²,
wherein the aliphatic portions of R¹ are optionally substituted with 1 to 5 R³ groups;
X^{a} is C₁-C₆ alkylene, C₁-C₆ heteroalkylene, C₃-C₇ cycloalkylene, or C₄-C₇ heterocycloalkylene, each of which is optionally substituted with 1 to 5 R³ groups;
each R² is independently H, C₁-C₆ alkyl, C₁-C₆ haloalkyl, C₁-C₆ heteroalkyl,
C₃-C₇ cycloalkyl, 4- to 7-membered heterocycloalkyl, or 5- to 6-membered heteroaryl,
wherein the aliphatic and aromatic portions of R² are optionally substituted with 1 to 5 R³ groups, and
wherein any two R² groups attached to the same nitrogen are optionally taken together to form a 4- to 7-membered heterocyclic ring, or any two R² groups attached to the same carbon atom are optionally taken together to form a 3- to 6-membered carbocyclic ring or a 4- to 6-membered heterocyclic ring optionally containing 1-3 heteroatoms selected from the group consisting of N, O, and S, wherein each carbocyclic or heterocyclic ring is optionally substituted with 1 to 5 R³ groups;
each R³ is independently C₁-C₆ alkyl, C₁-C₆-alkoxy, C₃-C₇ cycloalkyl, C₃-C₇ cycloalkoxy,
C₁-C₆ haloalkyl, C₁-C₆ alkylamino, C₁-C₆ dialkylamino, 4- to 7-membered heterocycloalkyl, 4- to 7-membered heterocycloalkoxy, 5- to 6-membered heteroaryl, F, Cl, —OH, —NH₂, —NHMe, —NMe₂, —SH, —SMe, —S(O)Me, —S(O)₂Me,
—S(O)(═NH)Me, —S(O)(═NMe)Me, —CN, —NO₂, —CHF₂, —CF₃, —CF₂Cl, —OCF₃, —OCHF₂, —SCF₃, —SCHF₂, —SF₅, —P(O)(Me)₂, or —N₃;
Ring A is C₆-C₁₀ aryl or 5- to 10-membered heteroaryl,
wherein the aromatic portions of Ring A are optionally substituted with 1 to 5 R⁴ groups, and
wherein when Ring A is C₆ aryl or 5- to 6-membered heteroaryl, then any two substituents attached to adjacent atoms of said aryl or heteroaryl are optionally taken together to form a 4- to 6-membered carbocyclic ring or a 4- to 6-membered heterocyclic ring optionally containing 1-3 heteroatoms selected from the group consisting of N, O, and S, wherein each carbocyclic or heterocyclic ring is optionally substituted with 1 to 5 R⁴ groups;
each R⁴ is independently F, Cl, Br, I, —SCF₃, —SCHF₂, —SF₅, —OCF₃, —OR⁶, —N(R⁶)R⁶, —N₃, —OCHF₂, —CF₃, —CF₂Cl, —CHF₂, —CN, —NO₂, —C(═O)C₁-C₆ alkyl, C₁-C₆ alkyl, C₁-C₆ haloalkyl, C₁-C₆ haloalkyl-OH, C₁-C₆ alkyl-OH, C₁-C₆ alkyl-OR⁶, C₁-C₆ alkylene-C₁-C₃ alkoxy, C₁-C₆ alkylene(—OH)—C₁-C₃ alkoxy, C₁-C₆ alkyl-CN, C₁-C₆ heteroalkyl, C₃-C₇ cycloalkyl, C₁-C₆ alkylene-C₃-C₇ cycloalkyl, C₁-C₆ alkylene(—OH)—C₃-C₇ cycloalkyl, or 5- to 6-membered heteroaryl;
each R⁵ is independently F, Cl, —SCF₃, —SCHF₂, —SF₅, —OCF₃, —OR⁶, —N(R⁶)R⁶, —N₃, —OCHF₂, —CF₃, —CF₂Cl, —CHF₂, —CN, —NO₂, C₁-C₆ alkyl, C₁-C₆ haloalkyl, C₁-C₆ heteroalkyl, or C₃-C₇ cycloalkyl,
wherein the aliphatic portions of R⁵ are optionally substituted with 1 to 3 R³ groups; and
each R⁶ is independently H, C₁-C₆ alkyl, C₁-C₆ haloalkyl, C₁-C₆ heteroalkyl,
C₃-C₇ cycloalkyl, C₆ aryl, 5- to 10-membered heteroaryl, or 4- to 7-membered heterocycloalkyl.

2. The compound of claim 1, wherein the compound of formula (I) is a compound of formula (I-1): 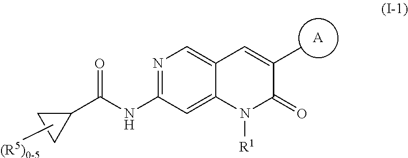
or a pharmaceutically acceptable salt, solvate, hydrate, or co-crystal thereof, or a mixture of any of the foregoing.

3. The compound of claim 1, wherein the compound of formula (I) is a compound of formula (I-2): 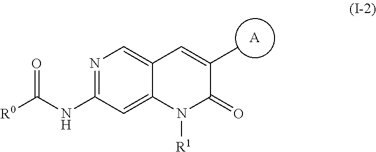
or a pharmaceutically acceptable salt, solvate, hydrate, or co-crystal thereof, or a mixture of any of the foregoing, wherein R⁰ is C₁-C₃ alkyl optionally substituted by 1 to 5 R⁵ groups.

4. The compound of any one of claims 1-3, wherein Ring A is selected from the group consisting of:

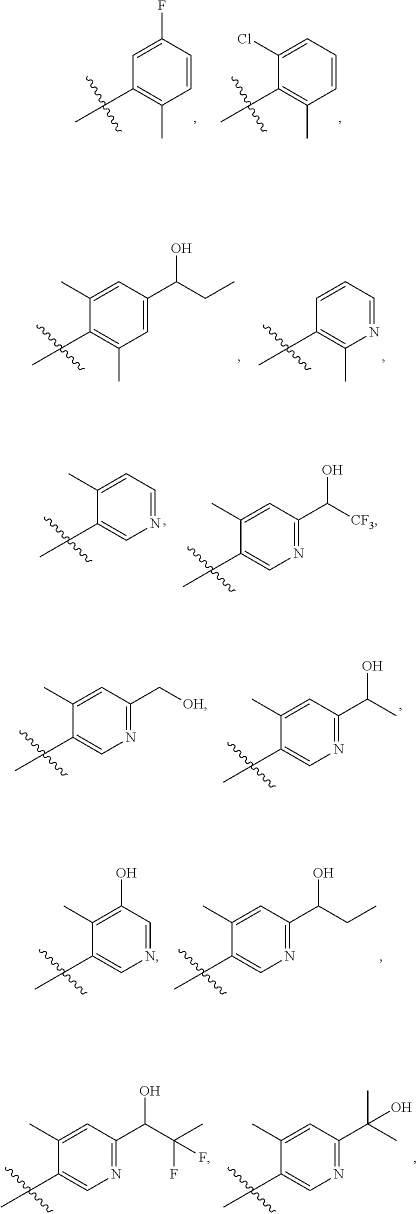

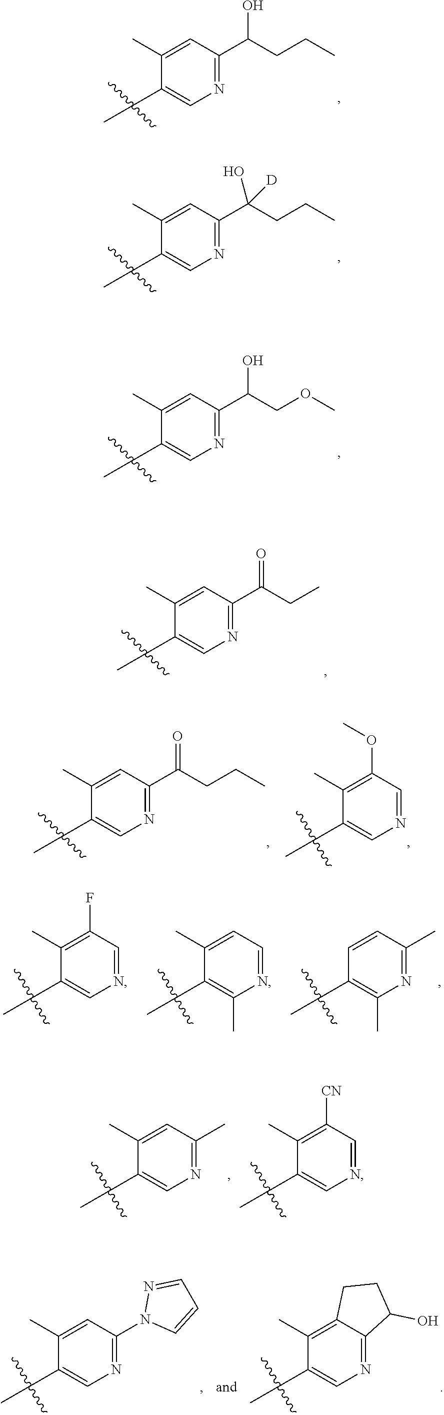

5. A compound of formula (I-1): 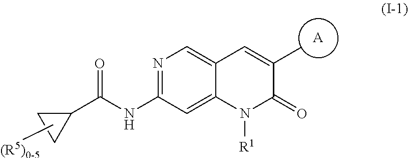
or a pharmaceutically acceptable salt, solvate, hydrate, or co-crystal thereof, or a mixture of any of the foregoing, wherein:
R¹ is H, C₁-C₆ alkyl, C₁-C₆ haloalkyl, C₁-C₆ heteroalkyl, C₃-C₇ cycloalkyl, C₁-C₆ alkyl-CN, 4- to 7-membered heterocycloalkyl, —(X^{a})₀₋₁—(C(R²)R²)₀₋₁—OR²,
—(X^{a})₀₋₁—(C(R²)R²)₀₋₁—N(R²)R², —(X^{a})₀₋₁—(C(R²)R²)₀₋₁—S(O)₂R²,
—(X^{a})—(C(R²)R²)₀₋₁—S(O)(═NR²)R², —(X^{a})—C(R²)R²—S(O)₂N(R²)R²,
—(X^{a})—C(R²)R²—S(O)R², —(X^{a})—C(R²)R²—N(R²)R²S(O)₂R², or
—(X^{a})—(C(R²)R²)₀₋₁—C(O)N(R²)R²,
wherein the aliphatic portions of R¹ are optionally substituted with 1 to 5 R³ groups;
X^{a} is C₁-C₆ alkylene, C₁-C₆ heteroalkylene, C₃-C₇ cycloalkylene, or C₄-C₇ heterocycloalkylene, each of which is optionally substituted with 1 to 5 R³ groups;
each R² is independently H, C₁-C₆ alkyl, C₁-C₆ haloalkyl, C₁-C₆ heteroalkyl,
C₃-C₇ cycloalkyl, 4- to 7-membered heterocycloalkyl, or 5- to 6-membered heteroaryl,
wherein the aliphatic and aromatic portions of R² are optionally substituted with 1 to 5 R³ groups, and
wherein any two R² groups attached to the same nitrogen are optionally taken together to form a 4- to 7-membered heterocyclic ring, or any two R² groups attached to the same carbon atom are optionally taken together to form a 3- to 6-membered carbocyclic ring or a 4- to 6-membered heterocyclic ring optionally containing 1-3 heteroatoms selected from the group consisting of N, O, and S, wherein each carbocyclic or heterocyclic ring is optionally substituted with 1 to 5 R³ groups;
each R³ is independently C₁-C₆ alkyl, C₁-C₆-alkoxy, C₃-C₇ cycloalkyl, C₃-C₇ cycloalkoxy,
C₁-C₆ haloalkyl, C₁-C₆ alkylamino, C₁-C₆ dialkylamino, 4- to 7-membered heterocycloalkyl, 4- to 7-membered heterocycloalkoxy, 5- to 6-membered heteroaryl, F, Cl, —OH, —NH₂, —NHMe, —NMe₂, —SH, —SMe, —S(O)Me, —S(O)₂Me,
—S(O)(═NH)Me, —S(O)(═NMe)Me, —CN, —NO₂, —CHF₂, —CF₃, —CF₂Cl, —OCF₃, —OCHF₂, —SCF₃, —SCHF₂, —SF₅, —P(O)(Me)₂, or —N₃;
Ring A is C₆-C₁₀ aryl or 5- to 10-membered heteroaryl,
wherein the aromatic portions of Ring A are optionally substituted with 1 to 5 R⁴ groups, and
wherein when Ring A is C₆ aryl or 5- to 6-membered heteroaryl, then any two substituents attached to adjacent atoms of said aryl or heteroaryl are optionally taken together to form a 4- to 6-membered carbocyclic ring or a 4- to 6-membered heterocyclic ring optionally containing 1-3 heteroatoms selected from the group consisting of N, O, and S, wherein each carbocyclic or heterocyclic ring is optionally substituted with 1 to 5 R⁴ groups;
each R⁴ is independently H, F, Cl, Br, I, —SCF₃, —SCHF₂, —SF₅, —OCF₃, —OR⁶, —N(R⁶)R⁶, —N₃, —OCHF₂, —CF₃, —CF₂Cl, —CHF₂, —CN, —NO₂, C₁-C₆ alkyl, C₁-C₆ haloalkyl, C₁-C₆ haloalkyl-OH, C₁-C₆ alkyl-OH, C₁-C₆ alkyl-CN, C₁-C₆ heteroalkyl, or C₃-C₇ cycloalkyl;
each R⁵ is independently F, Cl, —SCF₃, —SCHF₂, —SF₅, —OCF₃, —OR⁶, —N(R⁶)R⁶, —N₃, —OCHF₂, —CF₃, —CF₂Cl, —CHF₂, —CN, —NO₂, C₁-C₆ alkyl, C₁-C₆ haloalkyl, C₁-C₆ heteroalkyl, or C₃-C₇ cycloalkyl,
wherein the aliphatic portions of R⁵ are optionally substituted with 1 to 3 R³ groups; and
each R⁶ is independently H, C₁-C₆ alkyl, C₁-C₆ haloalkyl, C₁-C₆ heteroalkyl, C₃-C₇ cycloalkyl, C₆ aryl, 5- to 10-membered heteroaryl, or 4- to 7-membered heterocycloalkyl.

6. The compound of any one of claims 1-3 and 5, or a pharmaceutically acceptable salt, solvate, hydrate, or co-crystal thereof, or a mixture of any of the foregoing, wherein:
R¹ is H, C₁-C₆ alkyl, C₁-C₆ haloalkyl, C₁-C₆ heteroalkyl, C₃-C₇ cycloalkyl, C₁-C₆ alkyl-CN, 4- to 7-membered heterocycloalkyl, —(X^{a})₀₋₁—(C(R²)R²)₀₋₁—OR², or
—(X^{a})₀₋₁—(C(R²)R²)₀₋₁—N(R²)R²,
wherein the aliphatic portions of R¹ are optionally substituted with 1 to 5 R³ groups;
X^{a} is C₁-C₆ alkylene, C₁-C₆ heteroalkylene, C₃-C₇ cycloalkylene, or
C₄-C₇ heterocycloalkylene, each of which is optionally substituted with 1 to 5 R³ groups;
each R² is independently H, C₁-C₆ alkyl, C₁-C₆ haloalkyl, C₁-C₆ heteroalkyl,
C₃-C₇ cycloalkyl, 4- to 7-membered heterocycloalkyl, or 5- to 6-membered heteroaryl,
wherein the aliphatic and aromatic portions of R² are optionally substituted with 1 to 5 R³ groups;
each R³ is independently C₁-C₆ alkyl, C₁-C₆-alkoxy, C₃-C₇ cycloalkyl, C₃-C₇ cycloalkoxy,
C₁-C₆ haloalkyl, C₁-C₆ alkylamino, C₁-C₆ dialkylamino, 4- to 7-membered heterocycloalkyl, 4- to 7-membered heterocycloalkoxy, 5- to 6-membered heteroaryl, F, Cl, —OH, —NH₂, —NHMe, —NMe₂, —SMe, —S(O)Me, —S(O)₂Me, or —CN;
Ring A is C₆ aryl or 5- to 6-membered heteroaryl,
wherein the aromatic portions of Ring A are optionally substituted with 1 to 5 R⁴ groups;
each R⁴ is independently H, F, Cl, Br, I, —SCF₃, —SCHF₂, —SF₅, —OCF₃, —OR⁶, —N(R⁶)R⁶, —N₃, —OCHF₂, —CF₃, —CHF₂, —CN, —NO₂, C₁-C₆ alkyl, C₁-C₆ haloalkyl, C₁-C₆ haloalkyl-OH, C₁-C₆ alkyl-OH, C₁-C₆ alkyl-CN, C₁-C₆ heteroalkyl, or C₃-C₇ cycloalkyl;
each R⁵ is independently F, Cl, —SCF₃, —SCHF₂, —SF₅, —OCF₃, —OR⁶, —N(R⁶)R⁶, —N₃, —OCHF₂, —CF₃, —CHF₂, —CN, —NO₂, C₁-C₆ alkyl, C₁-C₆ haloalkyl, C₁-C₆ heteroalkyl, or C₃-C₇ cycloalkyl,
wherein the aliphatic portions of R⁵ are optionally substituted with 1 to 3 R³ groups; and
each R⁶ is independently H, C₁-C₆ alkyl, C₁-C₆ haloalkyl, C₁-C₆ heteroalkyl, C₃-C₇ cycloalkyl, C₆ aryl, 5- to 6-membered heteroaryl, or 4- to 7-membered heterocycloalkyl.

7. The compound of any one of claims 1-3 and 5-6, or a pharmaceutically acceptable salt, solvate, hydrate, or co-crystal thereof, or a mixture of any of the foregoing, wherein:
R¹ is H, C₁-C₆ alkyl, C₁-C₆ haloalkyl, C₁-C₆ alkyl-CN, or C₁-C₆ heteroalkyl,
wherein the aliphatic portions of R¹ are optionally substituted with 1 to 5 R³ groups;
each R³ is independently C₁-C₆ alkyl, C₁-C₆-alkoxy, C₃-C₇ cycloalkyl, C₃-C₇ cycloalkoxy,
C₁-C₆ haloalkyl, C₁-C₆ alkylamino, C₁-C₆ dialkylamino, 4- to 7-membered heterocycloalkyl, 4- to 7-membered heterocycloalkoxy, 5- to 6-membered heteroaryl, F, Cl, —OH, —NH₂, —NHMe, —NMe₂, —SMe, —S(O)Me, —S(O)₂Me, or —CN;
Ring A is C₆ aryl or 5- to 6-membered heteroaryl,
wherein the aromatic portions of Ring A are optionally substituted with 1 to 5 R⁴ groups;
each R⁴ is independently H, F, Cl, —OCF₃, —OR⁶, —N(R⁶)R⁶, —OCHF₂, —CF₃, —CHF₂, —CN,
C₁-C₆ alkyl, C₁-C₆ haloalkyl, C₁-C₆ haloalkyl-OH, C₁-C₆ alkyl-OH, C₁-C₆ alkyl-CN, C₁-C₆ heteroalkyl, or C₃-C₇ cycloalkyl;
each R⁵ is independently F, —OCF₃, —OR⁶, —N(R⁶)R⁶, —OCHF₂, —CF₃, —CN, C₁-C₆ alkyl, C₁-C₆ haloalkyl, or C₁-C₆ heteroalkyl,
wherein the aliphatic portions of R⁵ are optionally substituted with 1 to 3 R³ groups; and
each R⁶ is independently H, C₁-C₆ alkyl, C₁-C₆ haloalkyl, C₁-C₆ heteroalkyl, or C₃-C₇ cycloalkyl.

8. The compound of any one of claims 1-3 and 5-7, or a pharmaceutically acceptable salt, solvate, hydrate, or co-crystal thereof, or a mixture of any of the foregoing, wherein:
R¹ is H or C₁-C₆ alkyl optionally substituted with 1 to 5 R³ groups.

9. The compound of any one of claims 1-3 and 5-8, or a pharmaceutically acceptable salt, solvate, hydrate, or co-crystal thereof, or a mixture of any of the foregoing, wherein:
R¹ is —CH₃ or —CH₂CH₃.

10. The compound of any one of claims 1-3 and 5-9, or a pharmaceutically acceptable salt, solvate, hydrate, or co-crystal thereof, or a mixture of any of the foregoing, wherein:
Ring A is 6-membered heteroaryl optionally substituted with 1 to 4 R⁴ groups.

11. The compound of any one of claims 1-3 and 5-10, or a pharmaceutically acceptable salt, solvate, hydrate, or co-crystal thereof, or a mixture of any of the foregoing, wherein:
Ring A is pyridyl optionally substituted with 1 to 4 R⁴ groups.

12. The compound of any one of claims 1-3 and 5-11, or a pharmaceutically acceptable salt, solvate, hydrate, or co-crystal thereof, or a mixture of any of the foregoing, wherein:
Ring A is

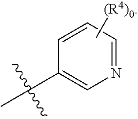

13. The compound of any one of claims 1-3 and 5-12, or a pharmaceutically acceptable salt, solvate, hydrate, or co-crystal thereof, or a mixture of any of the foregoing, wherein:
each R⁴ is independently F, C₁-C₆ alkyl, —OR⁶, —CN, —CH₂CN, —CH₂CH₂CN, C₁-C₆ alkyl-OH, or C₁-C₆ haloalkyl-OH; and
each R⁶ is independently H or C₁-C₆ alkyl.

14. The compound of any one of claims 1-3 and 5-13, or a pharmaceutically acceptable salt, solvate, hydrate, or co-crystal thereof, or a mixture of any of the foregoing, wherein:
each R⁴ is independently F, —CH₃, —OCH₃, —OH, —CN, —CH₂OH, —CH(OH)CH₃, —CH(OH)CH₂CH₃, —CH(OH)CF₂CH₃, —CH(OH)CH₂CH₂CH₃, —CD(OH)CH₂CH₂CH₃, —C(═O)CH₂CH₃, —C(═O)CH₂CH₂CH₃, —CH(OH)CH₂OCH₃, —C(CH₃)₂OH, or —CH(OH)CF₃.

15. The compound of any one of claims 1-3 and 5-14, or a pharmaceutically acceptable salt, solvate, hydrate, or co-crystal thereof, or a mixture of any of the foregoing, wherein:
each R⁴ is independently F, —CH₃, —OCH₃, —OH, —CN, —CH₂OH, —CH(OH)CH₃, —CH(OH)CH₂CH₃, —C(CH₃)₂OH, or —CH(OH)CF₃.

16. The compound of any one of claims 1-15, or a pharmaceutically acceptable salt, solvate, hydrate, or co-crystal thereof, or a mixture of any of the foregoing, wherein:
Ring A is selected from the group consisting of:

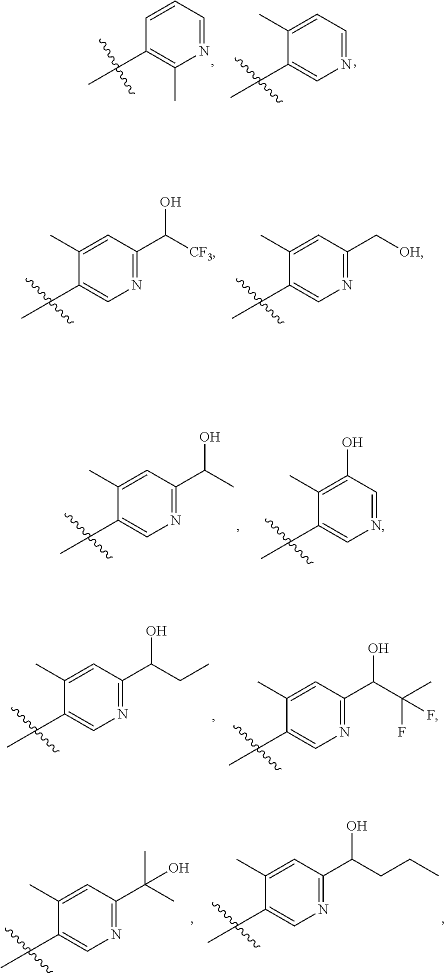

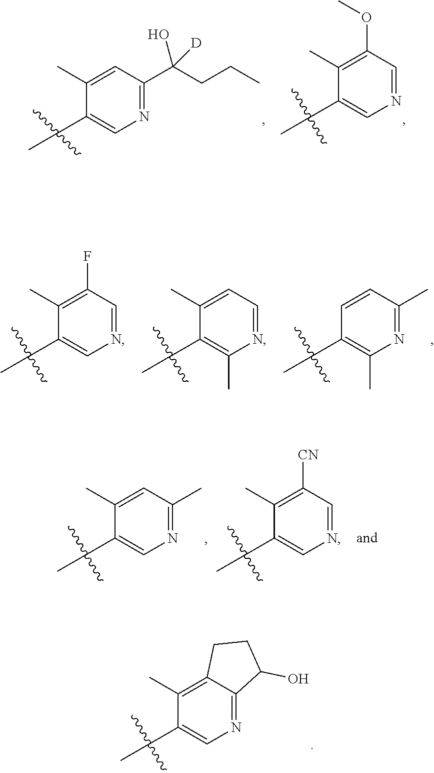

17. The compound of any one of claims 1-16, or a pharmaceutically acceptable salt, solvate, hydrate, or co-crystal thereof, or a mixture of any of the foregoing, wherein:
Ring A is selected from the group consisting of:

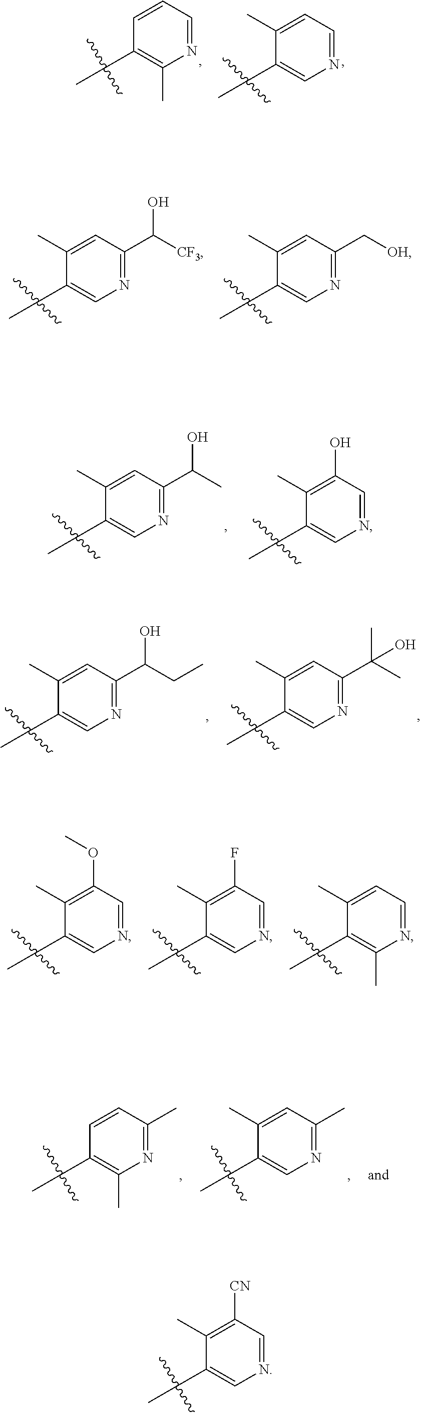

18. The compound of any one of claims 1-17, or a pharmaceutically acceptable salt, solvate, hydrate, or co-crystal thereof, or a mixture of any of the foregoing, wherein:
R⁵, if present, is F.

19. The compound of any one of claims 1-2 and 4-18, or a pharmaceutically acceptable salt, solvate, hydrate, or co-crystal thereof, or a mixture of any of the foregoing, wherein: 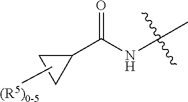
of formula (I) is

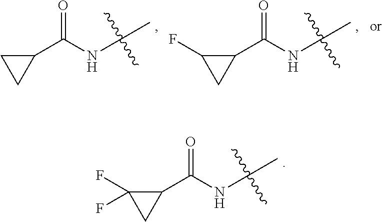 [SMILES: *NC(=O)C1CC1 ; *NC(=O)C1CC1F ; *NC(=O)C1CC1(F)F]

20. A compound, or pharmaceutically acceptable salt, solvate, hydrate, or co-crystal thereof, or a mixture of any of the foregoing, which is:

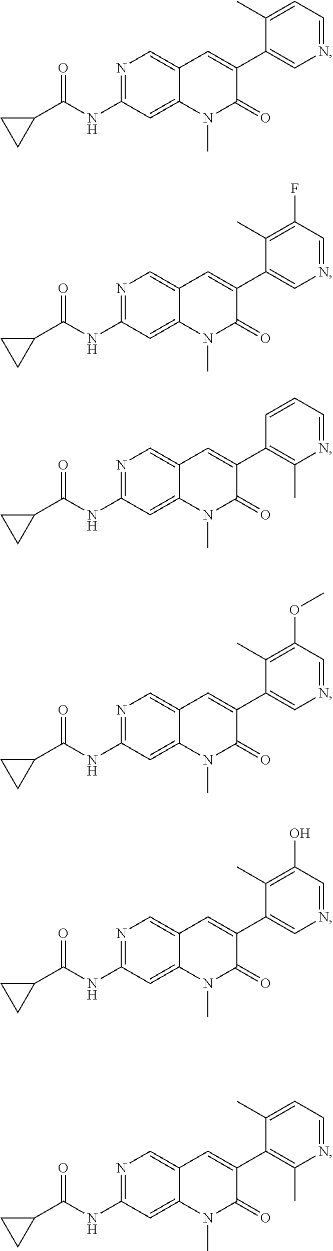 [SMILES: Cc1ccncc1-c1cc2cnc(NC(=O)C3CC3)cc2n(C)c1=O ; Cc1c(F)cncc1-c1cc2cnc(NC(=O)C3CC3)cc2n(C)c1=O ; Cc1ncccc1-c1cc2cnc(NC(=O)C3CC3)cc2n(C)c1=O ; COc1cncc(-c2cc3cnc(NC(=O)C4CC4)cc3n(C)c2=O)c1C ; Cc1c(O)cncc1-c1cc2cnc(NC(=O)C3CC3)cc2n(C)c1=O ; Cc1ccnc(C)c1-c1cc2cnc(NC(=O)C3CC3)cc2n(C)c1=O]

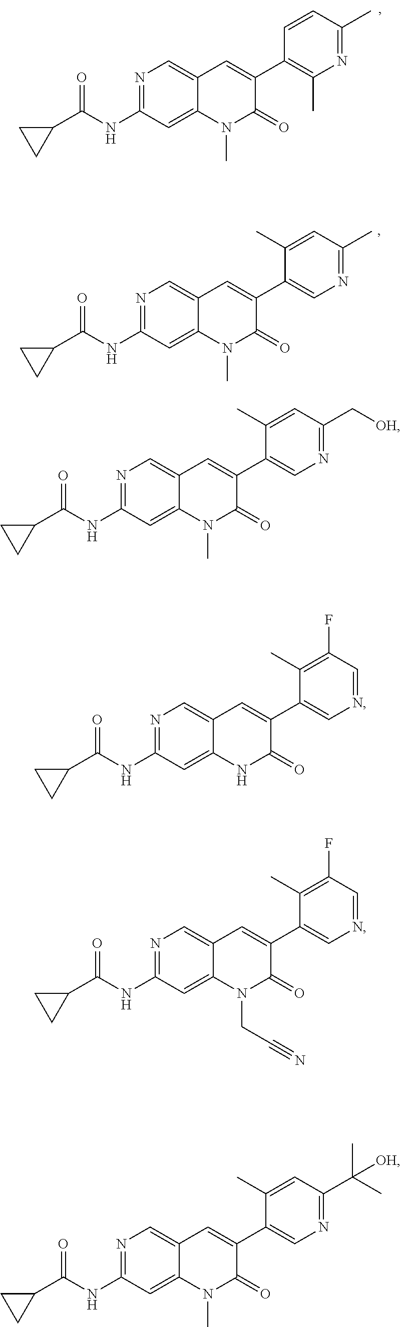

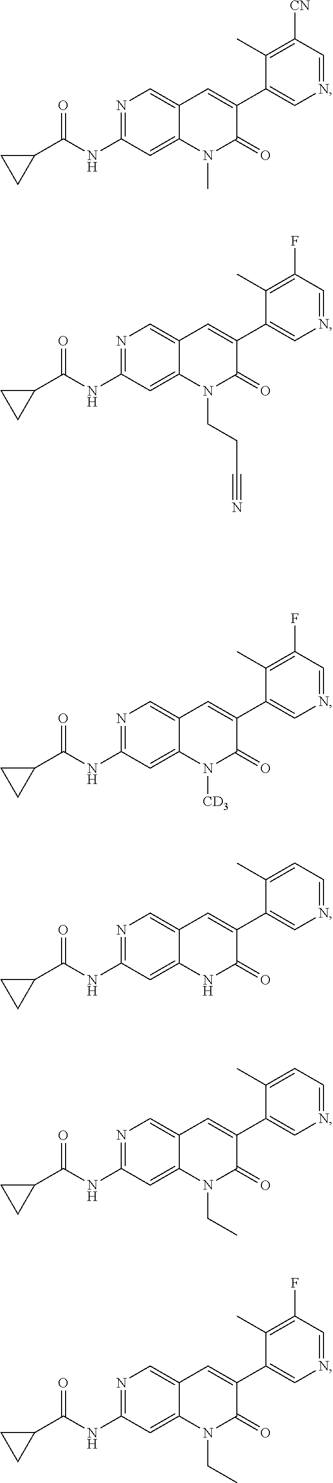 [SMILES: Cc1c(C#N)cncc1-c1cc2cnc(NC(=O)C3CC3)cc2n(C)c1=O ; Cc1c(F)cncc1-c1cc2cnc(NC(=O)C3CC3)cc2n(CCC#N)c1=O ; [2H]C([2H])([2H])n1c(=O)c(-c2cncc(F)c2C)cc2cnc(NC(=O)C3CC3)cc21 ; Cc1ccncc1-c1cc2cnc(NC(=O)C3CC3)cc2[nH]c1=O ; CCn1c(=O)c(-c2cnccc2C)cc2cnc(NC(=O)C3CC3)cc21 ; CCn1c(=O)c(-c2cncc(F)c2C)cc2cnc(NC(=O)C3CC3)cc21]

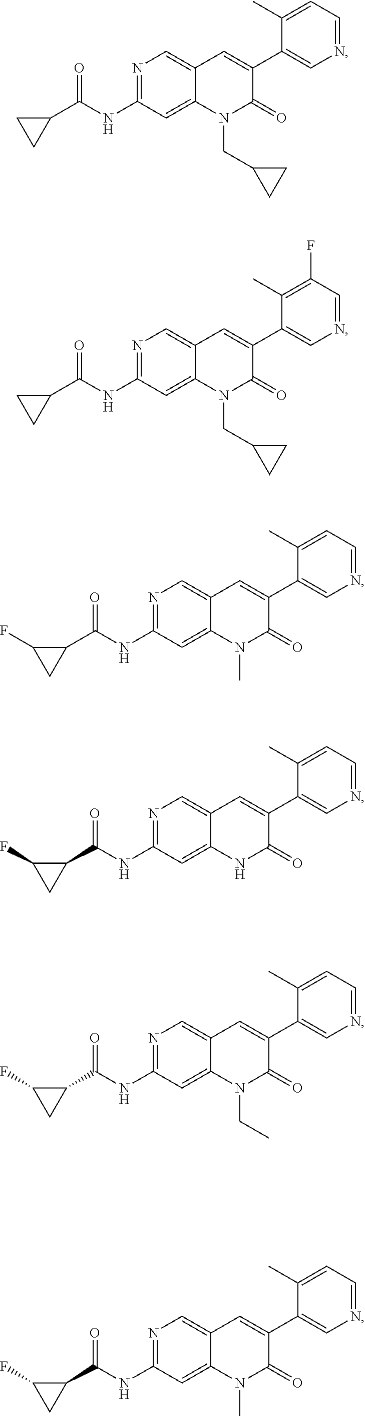 [SMILES: Cc1ccncc1-c1cc2cnc(NC(=O)C3CC3)cc2n(CC2CC2)c1=O ; Cc1c(F)cncc1-c1cc2cnc(NC(=O)C3CC3)cc2n(CC2CC2)c1=O ; Cc1ccncc1-c1cc2cnc(NC(=O)C3CC3F)cc2n(C)c1=O ; Cc1ccncc1-c1cc2cnc(NC(=O)[C@H]3C[C@H]3F)cc2[nH]c1=O ; CCn1c(=O)c(-c2cnccc2C)cc2cnc(NC(=O)[C@@H]3C[C@@H]3F)cc21 ; Cc1ccncc1-c1cc2cnc(NC(=O)[C@H]3C[C@@H]3F)cc2n(C)c1=O]

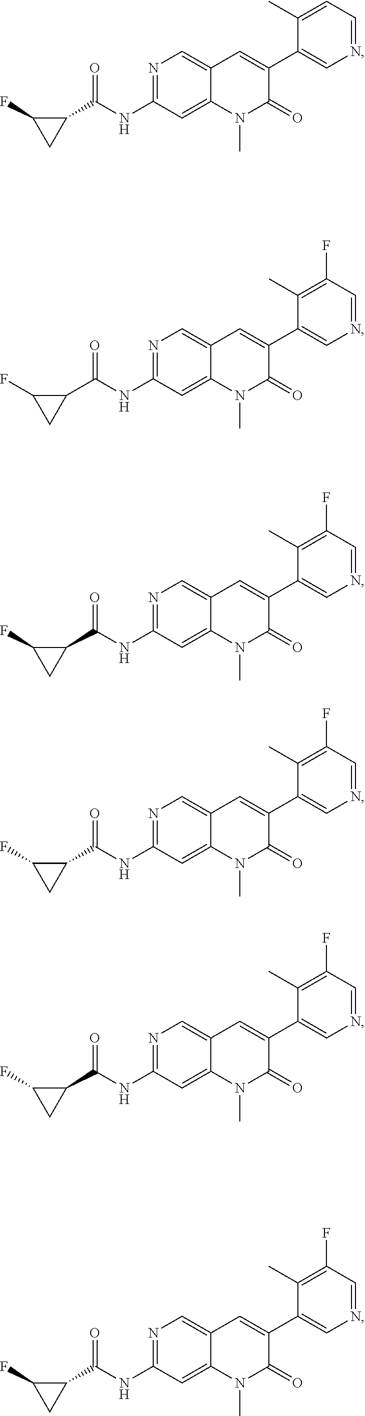 [SMILES: Cc1ccncc1-c1cc2cnc(NC(=O)[C@@H]3C[C@H]3F)cc2n(C)c1=O ; Cc1c(F)cncc1-c1cc2cnc(NC(=O)C3CC3F)cc2n(C)c1=O ; Cc1c(F)cncc1-c1cc2cnc(NC(=O)[C@H]3C[C@H]3F)cc2n(C)c1=O ; Cc1c(F)cncc1-c1cc2cnc(NC(=O)[C@@H]3C[C@@H]3F)cc2n(C)c1=O ; Cc1c(F)cncc1-c1cc2cnc(NC(=O)[C@H]3C[C@@H]3F)cc2n(C)c1=O ; Cc1c(F)cncc1-c1cc2cnc(NC(=O)[C@@H]3C[C@H]3F)cc2n(C)c1=O]

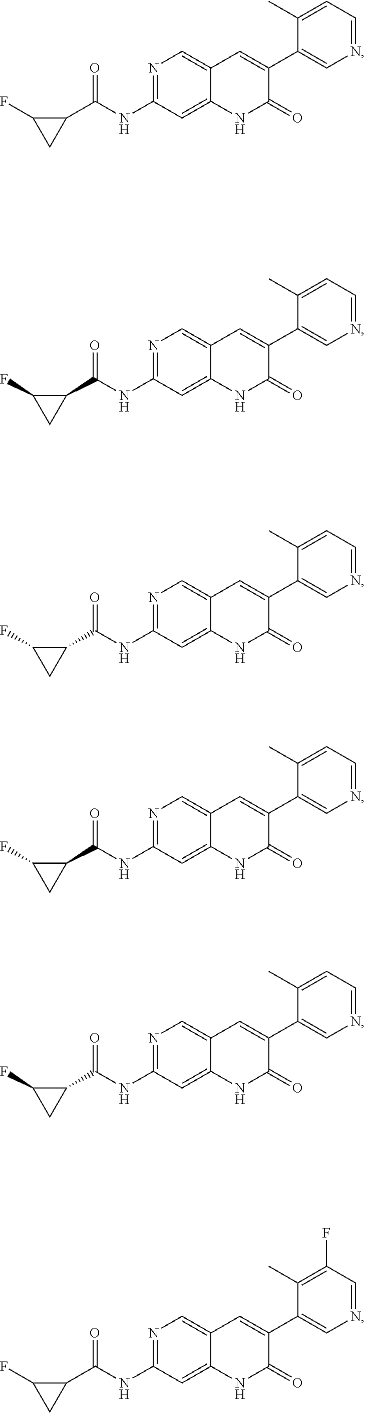 [SMILES: Cc1ccncc1-c1cc2cnc(NC(=O)C3CC3F)cc2[nH]c1=O ; Cc1ccncc1-c1cc2cnc(NC(=O)[C@H]3C[C@H]3F)cc2[nH]c1=O ; Cc1ccncc1-c1cc2cnc(NC(=O)[C@@H]3C[C@@H]3F)cc2[nH]c1=O ; Cc1ccncc1-c1cc2cnc(NC(=O)[C@H]3C[C@@H]3F)cc2[nH]c1=O ; Cc1ccncc1-c1cc2cnc(NC(=O)[C@@H]3C[C@H]3F)cc2[nH]c1=O ; Cc1c(F)cncc1-c1cc2cnc(NC(=O)C3CC3F)cc2[nH]c1=O]

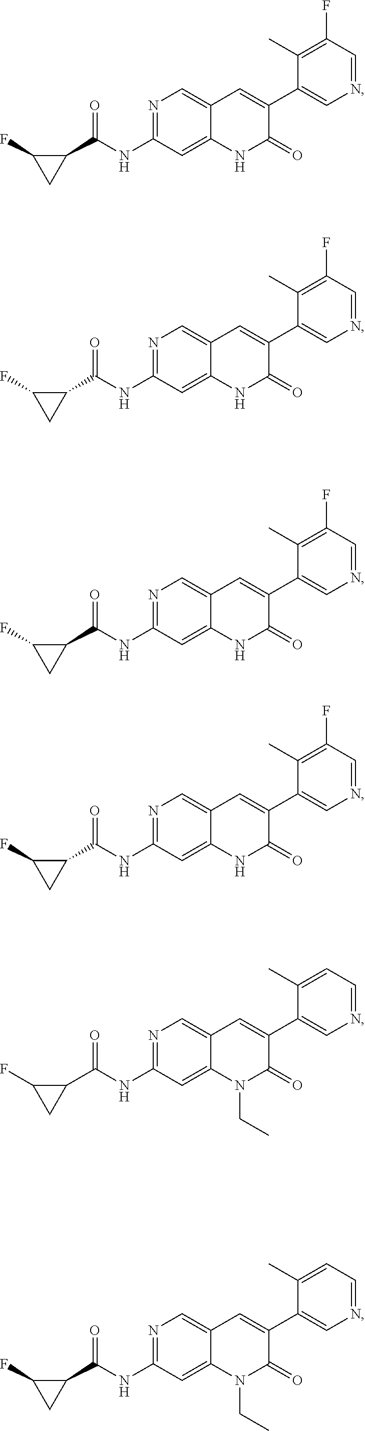 [SMILES: Cc1c(F)cncc1-c1cc2cnc(NC(=O)[C@H]3C[C@H]3F)cc2[nH]c1=O ; Cc1c(F)cncc1-c1cc2cnc(NC(=O)[C@@H]3C[C@@H]3F)cc2[nH]c1=O ; Cc1c(F)cncc1-c1cc2cnc(NC(=O)[C@H]3C[C@@H]3F)cc2[nH]c1=O ; Cc1c(F)cncc1-c1cc2cnc(NC(=O)[C@@H]3C[C@H]3F)cc2[nH]c1=O ; CCn1c(=O)c(-c2cnccc2C)cc2cnc(NC(=O)C3CC3F)cc21 ; CCn1c(=O)c(-c2cnccc2C)cc2cnc(NC(=O)[C@H]3C[C@H]3F)cc21]

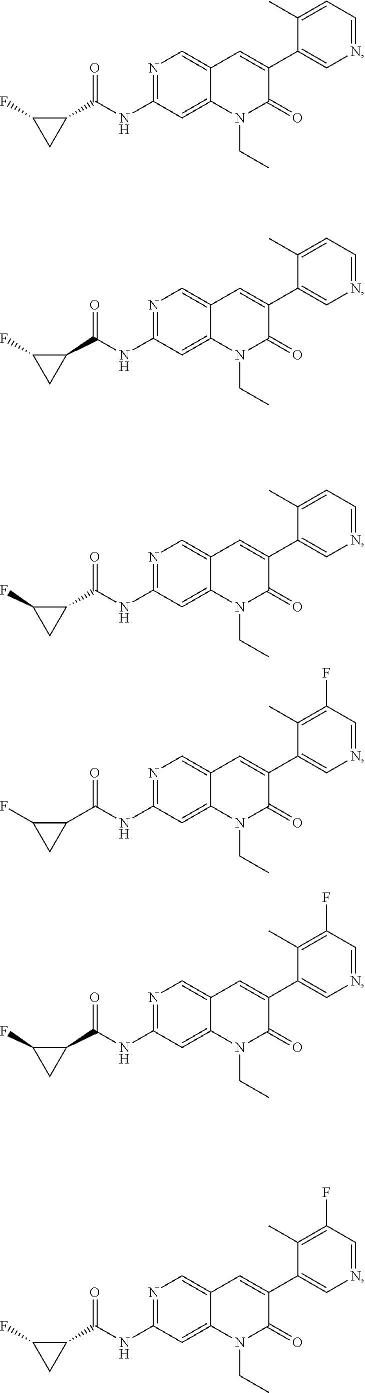 [SMILES: CCn1c(=O)c(-c2cnccc2C)cc2cnc(NC(=O)[C@@H]3C[C@@H]3F)cc21 ; CCn1c(=O)c(-c2cnccc2C)cc2cnc(NC(=O)[C@H]3C[C@@H]3F)cc21 ; CCn1c(=O)c(-c2cnccc2C)cc2cnc(NC(=O)[C@@H]3C[C@H]3F)cc21 ; CCn1c(=O)c(-c2cncc(F)c2C)cc2cnc(NC(=O)C3CC3F)cc21 ; CCn1c(=O)c(-c2cncc(F)c2C)cc2cnc(NC(=O)[C@H]3C[C@H]3F)cc21 ; CCn1c(=O)c(-c2cncc(F)c2C)cc2cnc(NC(=O)[C@@H]3C[C@@H]3F)cc21]

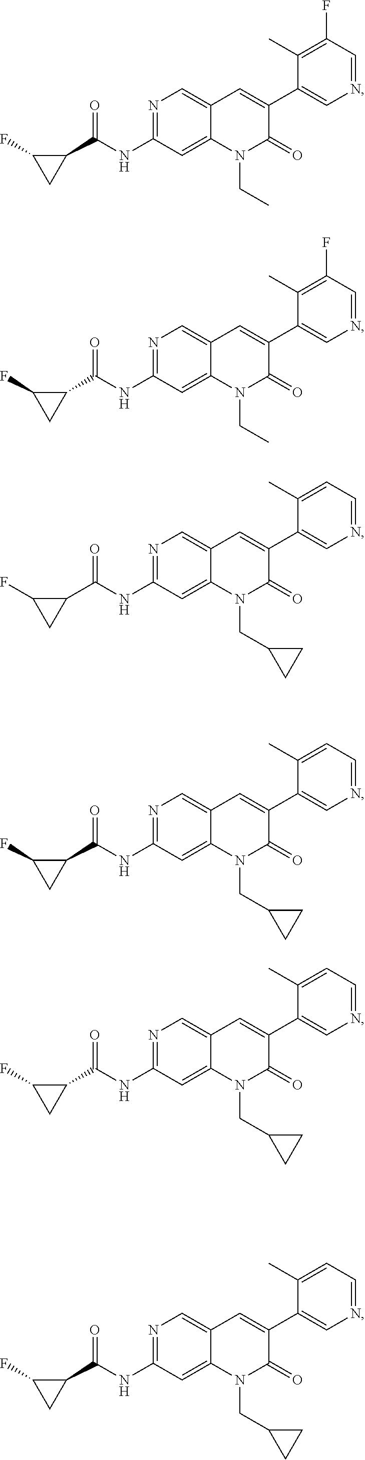

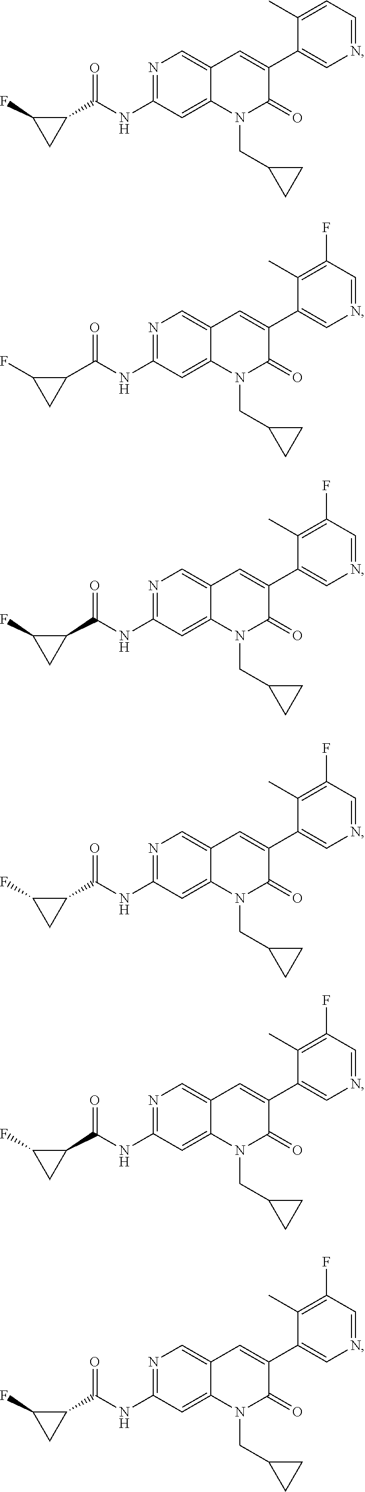 [SMILES: Cc1ccncc1-c1cc2cnc(NC(=O)[C@@H]3C[C@H]3F)cc2n(CC2CC2)c1=O ; Cc1c(F)cncc1-c1cc2cnc(NC(=O)C3CC3F)cc2n(CC2CC2)c1=O ; Cc1c(F)cncc1-c1cc2cnc(NC(=O)[C@H]3C[C@H]3F)cc2n(CC2CC2)c1=O ; Cc1c(F)cncc1-c1cc2cnc(NC(=O)[C@@H]3C[C@@H]3F)cc2n(CC2CC2)c1=O ; Cc1c(F)cncc1-c1cc2cnc(NC(=O)[C@H]3C[C@@H]3F)cc2n(CC2CC2)c1=O ; Cc1c(F)cncc1-c1cc2cnc(NC(=O)[C@@H]3C[C@H]3F)cc2n(CC2CC2)c1=O]

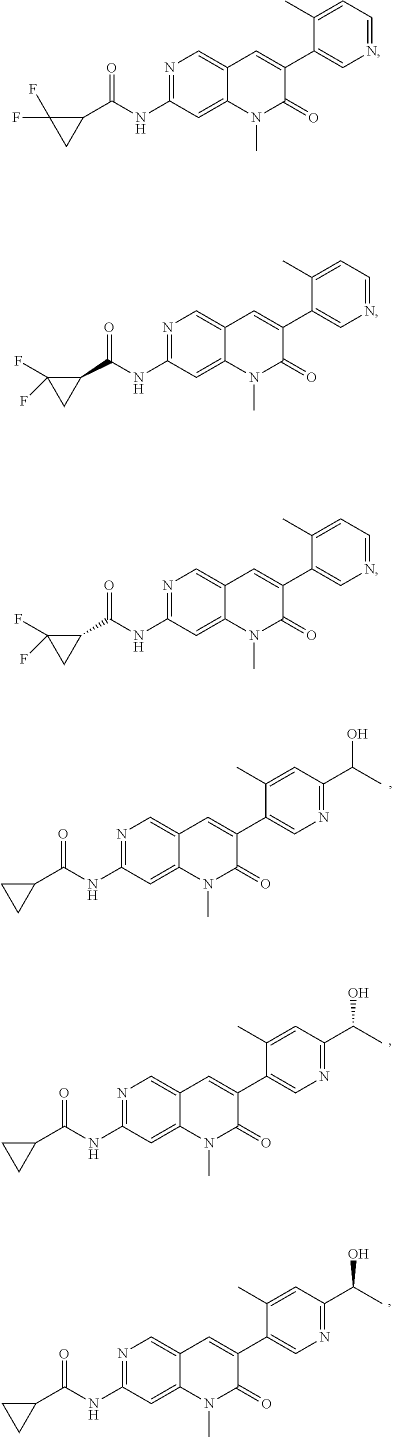 [SMILES: Cc1ccncc1-c1cc2cnc(NC(=O)C3CC3(F)F)cc2n(C)c1=O ; Cc1ccncc1-c1cc2cnc(NC(=O)[C@H]3CC3(F)F)cc2n(C)c1=O ; Cc1ccncc1-c1cc2cnc(NC(=O)[C@@H]3CC3(F)F)cc2n(C)c1=O ; Cc1cc(C(C)O)ncc1-c1cc2cnc(NC(=O)C3CC3)cc2n(C)c1=O ; Cc1cc([C@@H](C)O)ncc1-c1cc2cnc(NC(=O)C3CC3)cc2n(C)c1=O ; Cc1cc([C@H](C)O)ncc1-c1cc2cnc(NC(=O)C3CC3)cc2n(C)c1=O]

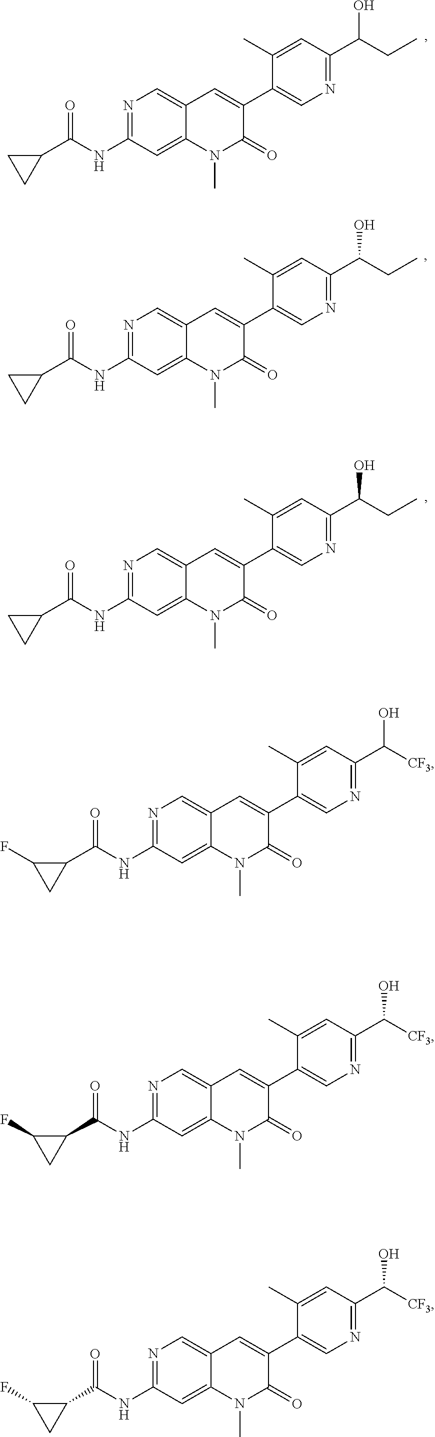 [SMILES: CCC(O)c1cc(C)c(-c2cc3cnc(NC(=O)C4CC4)cc3n(C)c2=O)cn1 ; CC[C@@H](O)c1cc(C)c(-c2cc3cnc(NC(=O)C4CC4)cc3n(C)c2=O)cn1 ; CC[C@H](O)c1cc(C)c(-c2cc3cnc(NC(=O)C4CC4)cc3n(C)c2=O)cn1 ; Cc1cc(C(O)C(F)(F)F)ncc1-c1cc2cnc(NC(=O)C3CC3F)cc2n(C)c1=O ; Cc1cc([C@H](O)C(F)(F)F)ncc1-c1cc2cnc(NC(=O)[C@H]3C[C@H]3F)cc2n(C)c1=O ; Cc1cc([C@H](O)C(F)(F)F)ncc1-c1cc2cnc(NC(=O)[C@@H]3C[C@@H]3F)cc2n(C)c1=O]

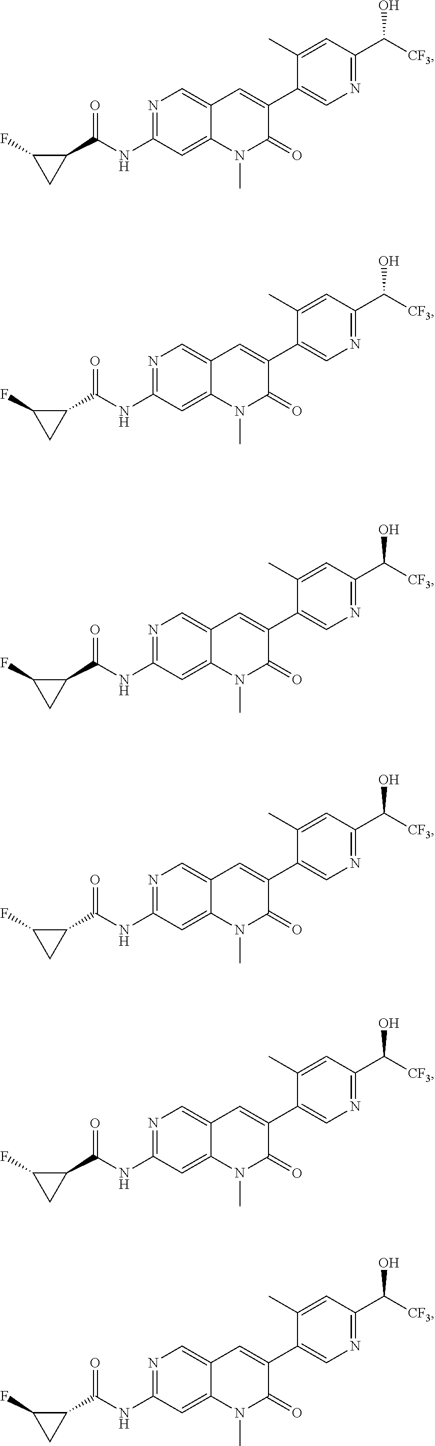 [SMILES: Cc1cc([C@H](O)C(F)(F)F)ncc1-c1cc2cnc(NC(=O)[C@H]3C[C@@H]3F)cc2n(C)c1=O ; Cc1cc([C@H](O)C(F)(F)F)ncc1-c1cc2cnc(NC(=O)[C@@H]3C[C@H]3F)cc2n(C)c1=O ; Cc1cc([C@@H](O)C(F)(F)F)ncc1-c1cc2cnc(NC(=O)[C@H]3C[C@H]3F)cc2n(C)c1=O ; Cc1cc([C@@H](O)C(F)(F)F)ncc1-c1cc2cnc(NC(=O)[C@@H]3C[C@@H]3F)cc2n(C)c1=O ; Cc1cc([C@@H](O)C(F)(F)F)ncc1-c1cc2cnc(NC(=O)[C@H]3C[C@@H]3F)cc2n(C)c1=O ; Cc1cc([C@@H](O)C(F)(F)F)ncc1-c1cc2cnc(NC(=O)[C@@H]3C[C@H]3F)cc2n(C)c1=O]

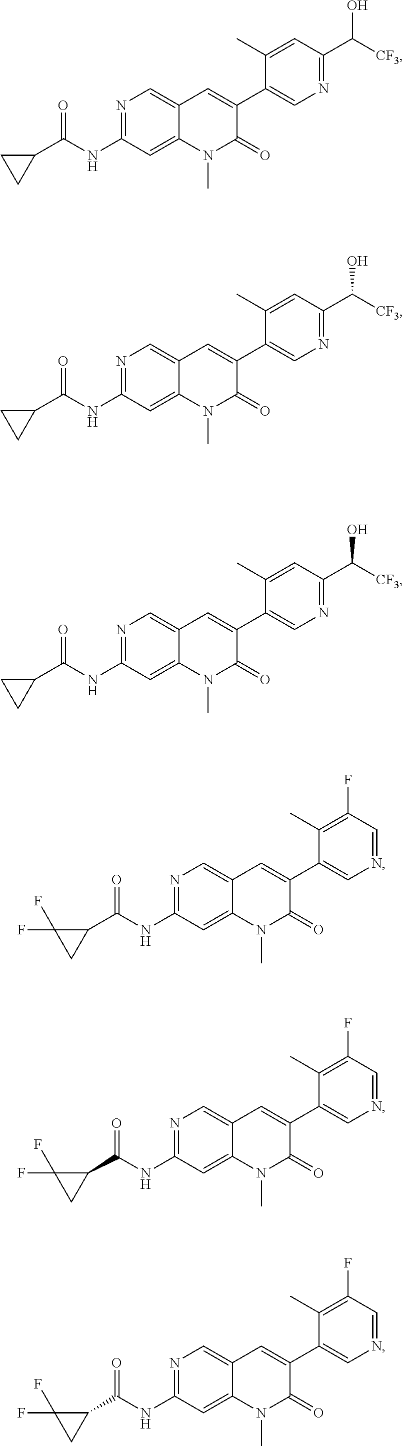 [SMILES: Cc1cc(C(O)C(F)(F)F)ncc1-c1cc2cnc(NC(=O)C3CC3)cc2n(C)c1=O ; Cc1cc([C@H](O)C(F)(F)F)ncc1-c1cc2cnc(NC(=O)C3CC3)cc2n(C)c1=O ; Cc1cc([C@@H](O)C(F)(F)F)ncc1-c1cc2cnc(NC(=O)C3CC3)cc2n(C)c1=O ; Cc1c(F)cncc1-c1cc2cnc(NC(=O)C3CC3(F)F)cc2n(C)c1=O ; Cc1c(F)cncc1-c1cc2cnc(NC(=O)[C@H]3CC3(F)F)cc2n(C)c1=O ; Cc1c(F)cncc1-c1cc2cnc(NC(=O)[C@@H]3CC3(F)F)cc2n(C)c1=O]

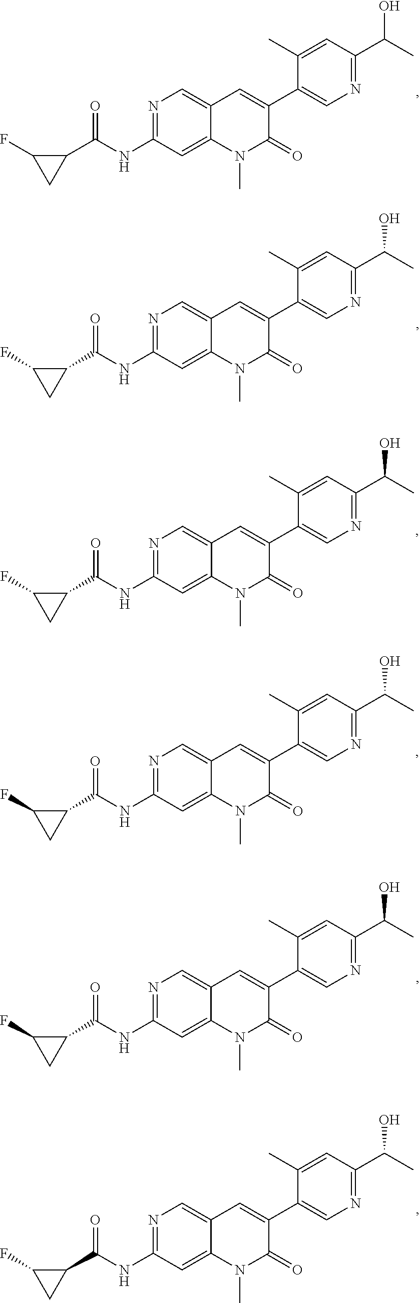 [SMILES: Cc1cc(C(C)O)ncc1-c1cc2cnc(NC(=O)C3CC3F)cc2n(C)c1=O ; Cc1cc([C@@H](C)O)ncc1-c1cc2cnc(NC(=O)[C@@H]3C[C@@H]3F)cc2n(C)c1=O ; Cc1cc([C@H](C)O)ncc1-c1cc2cnc(NC(=O)[C@@H]3C[C@@H]3F)cc2n(C)c1=O ; Cc1cc([C@@H](C)O)ncc1-c1cc2cnc(NC(=O)[C@@H]3C[C@H]3F)cc2n(C)c1=O ; Cc1cc([C@H](C)O)ncc1-c1cc2cnc(NC(=O)[C@@H]3C[C@H]3F)cc2n(C)c1=O ; Cc1cc([C@@H](C)O)ncc1-c1cc2cnc(NC(=O)[C@H]3C[C@@H]3F)cc2n(C)c1=O]

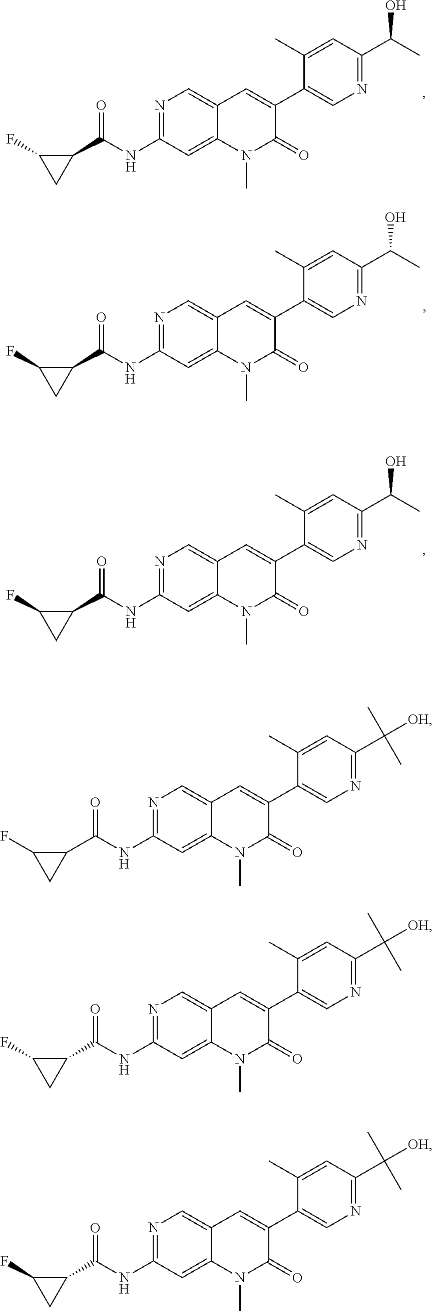 [SMILES: Cc1cc([C@H](C)O)ncc1-c1cc2cnc(NC(=O)[C@H]3C[C@@H]3F)cc2n(C)c1=O ; Cc1cc([C@@H](C)O)ncc1-c1cc2cnc(NC(=O)[C@H]3C[C@H]3F)cc2n(C)c1=O ; Cc1cc([C@H](C)O)ncc1-c1cc2cnc(NC(=O)[C@H]3C[C@H]3F)cc2n(C)c1=O ; Cc1cc(C(C)(C)O)ncc1-c1cc2cnc(NC(=O)C3CC3F)cc2n(C)c1=O ; Cc1cc(C(C)(C)O)ncc1-c1cc2cnc(NC(=O)[C@@H]3C[C@@H]3F)cc2n(C)c1=O ; Cc1cc(C(C)(C)O)ncc1-c1cc2cnc(NC(=O)[C@@H]3C[C@H]3F)cc2n(C)c1=O]

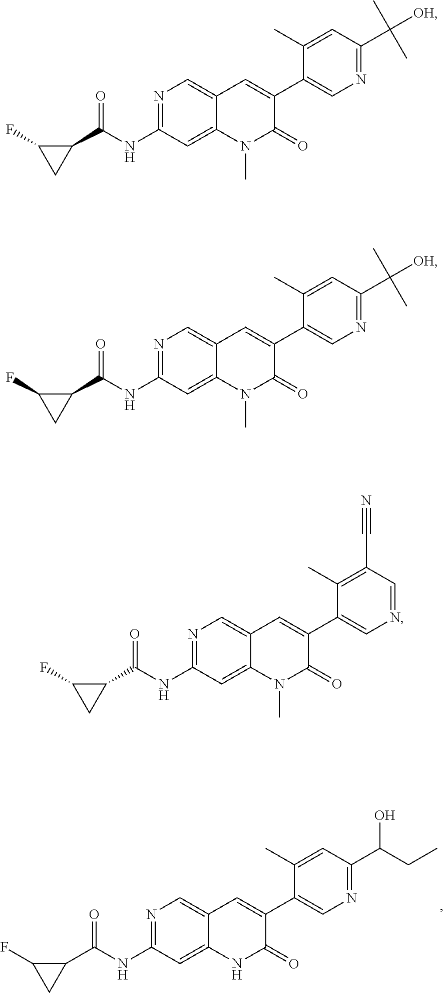 [SMILES: Cc1cc(C(C)(C)O)ncc1-c1cc2cnc(NC(=O)[C@H]3C[C@@H]3F)cc2n(C)c1=O ; Cc1cc(C(C)(C)O)ncc1-c1cc2cnc(NC(=O)[C@H]3C[C@H]3F)cc2n(C)c1=O ; Cc1c(C#N)cncc1-c1cc2cnc(NC(=O)[C@@H]3C[C@@H]3F)cc2n(C)c1=O ; CCC(O)c1cc(C)c(-c2cc3cnc(NC(=O)C4CC4F)cc3[nH]c2=O)cn1]

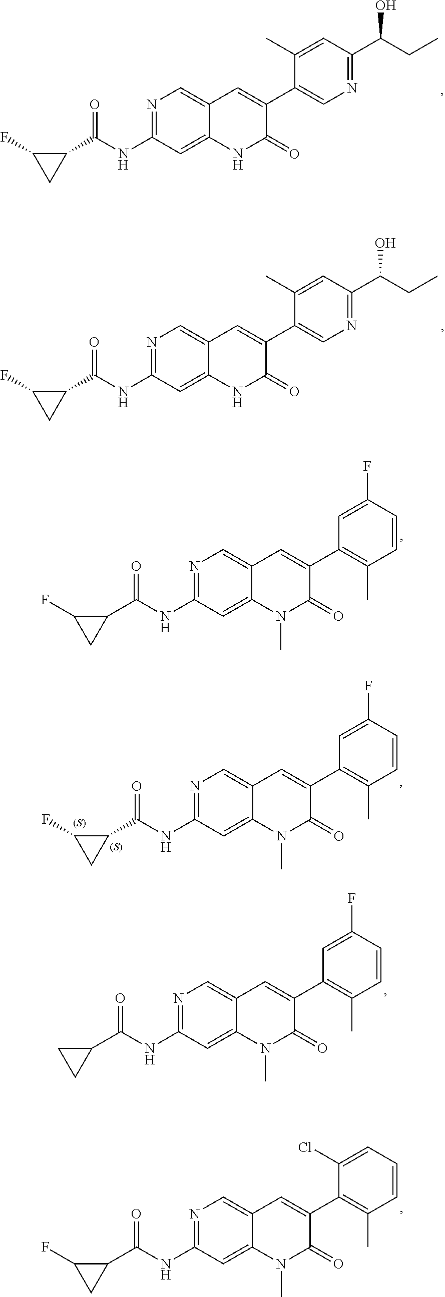

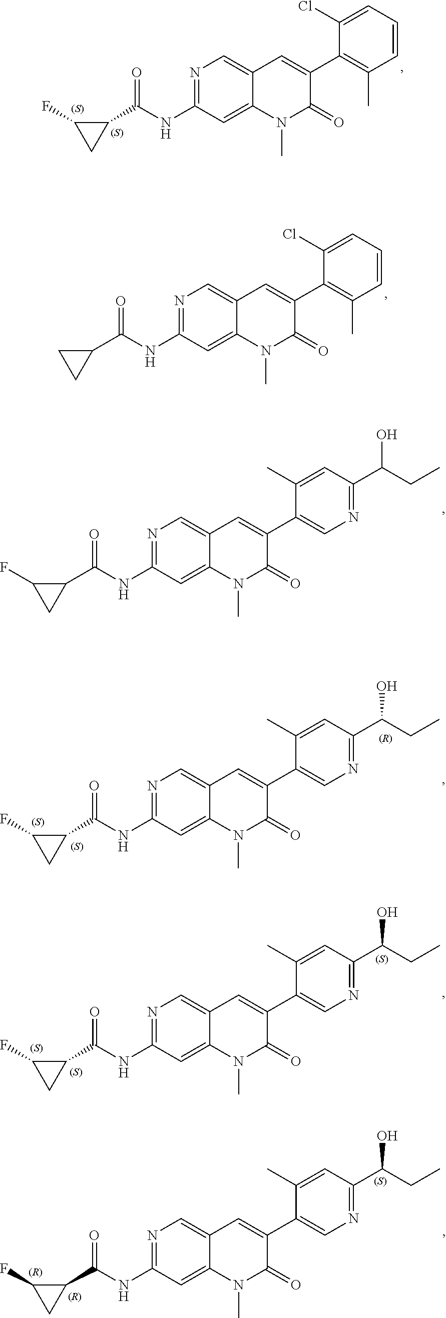

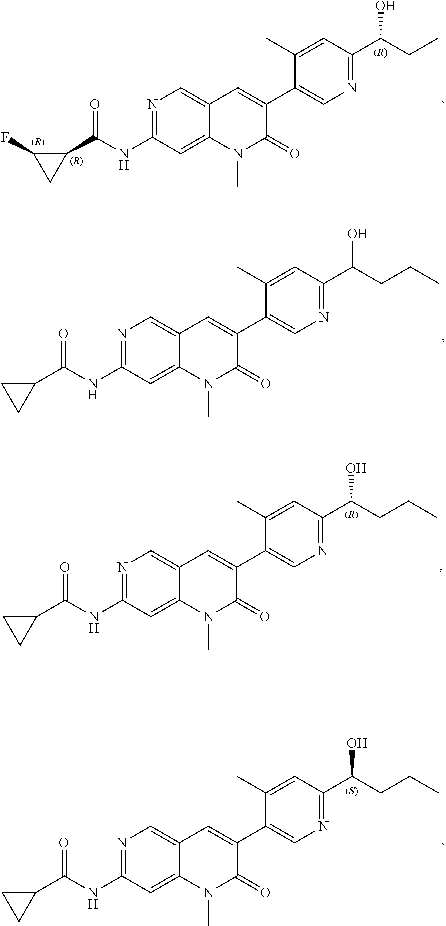

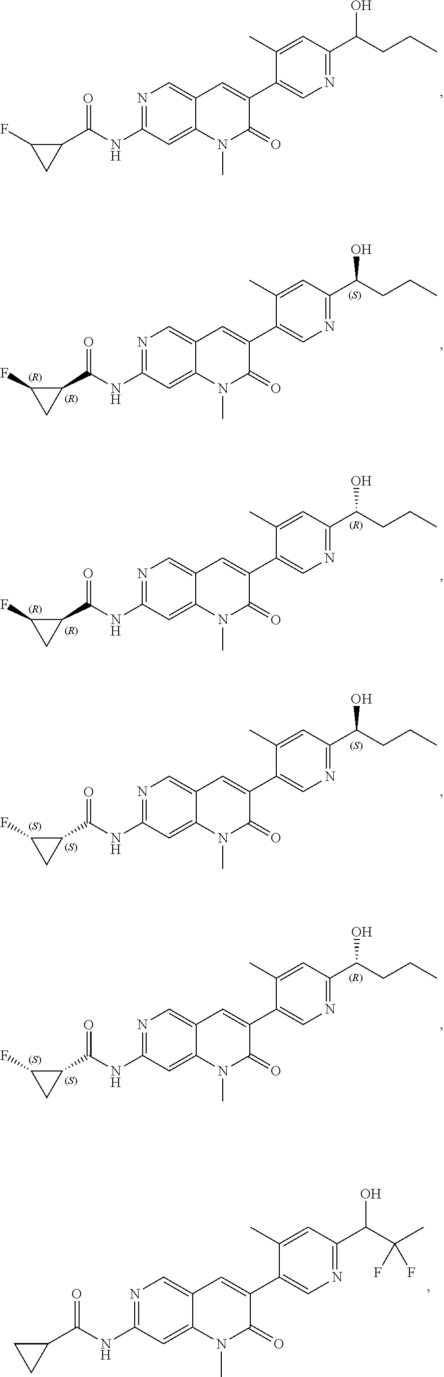

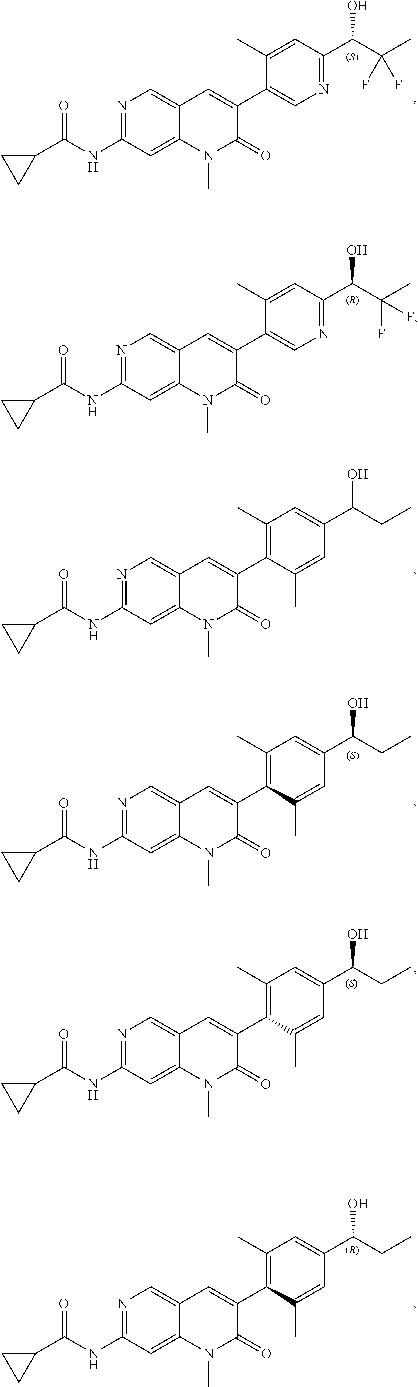

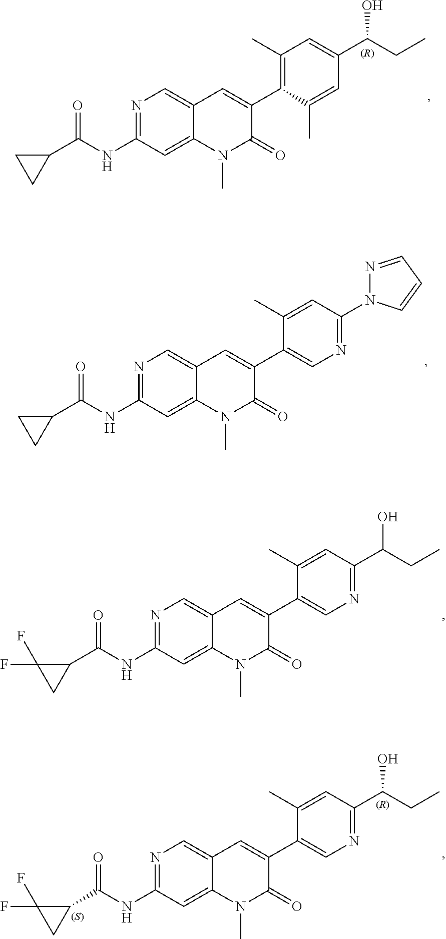

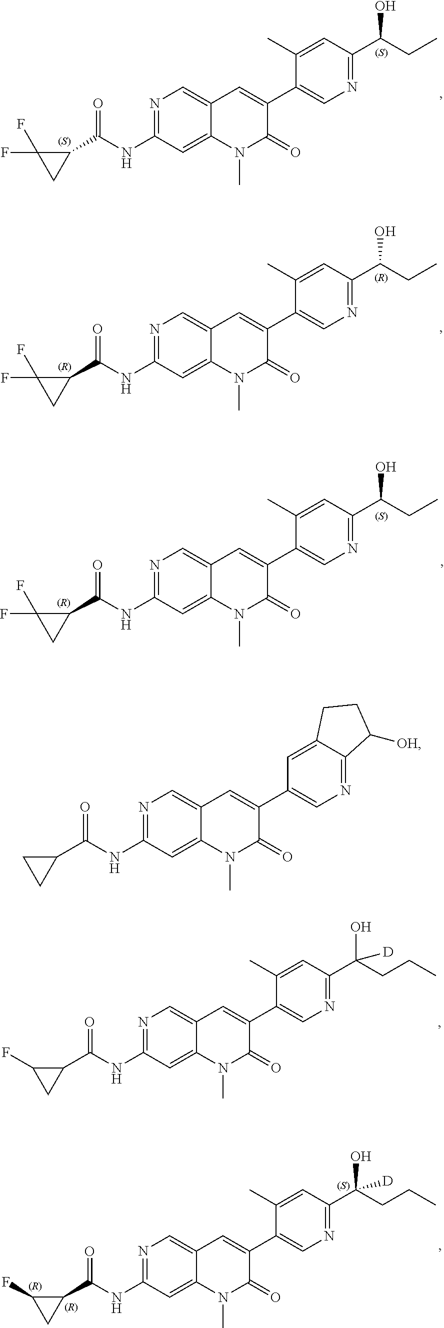

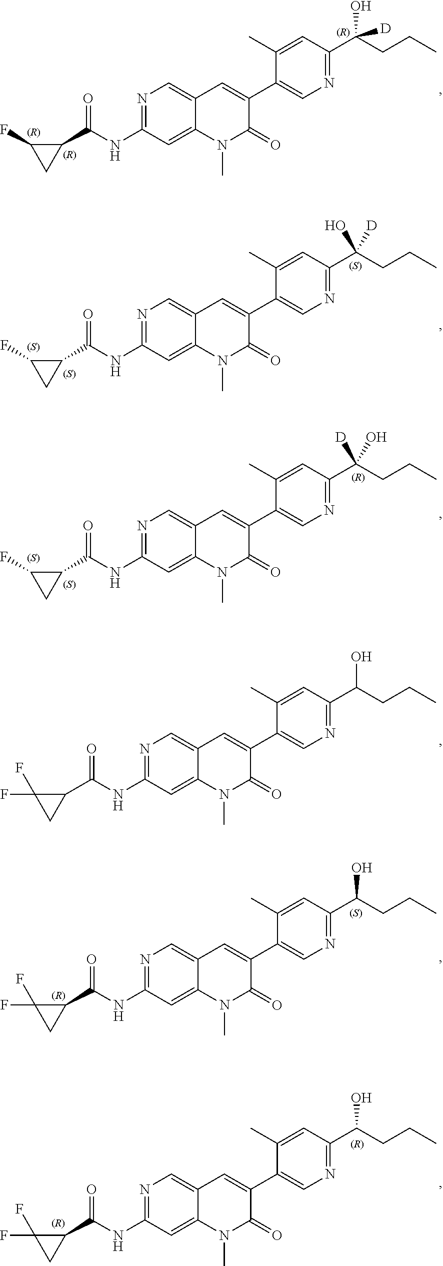

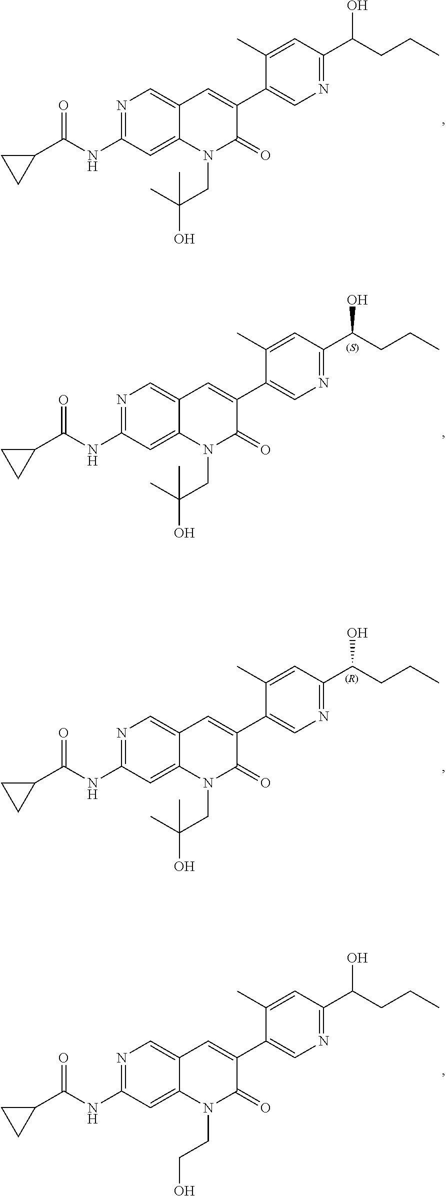

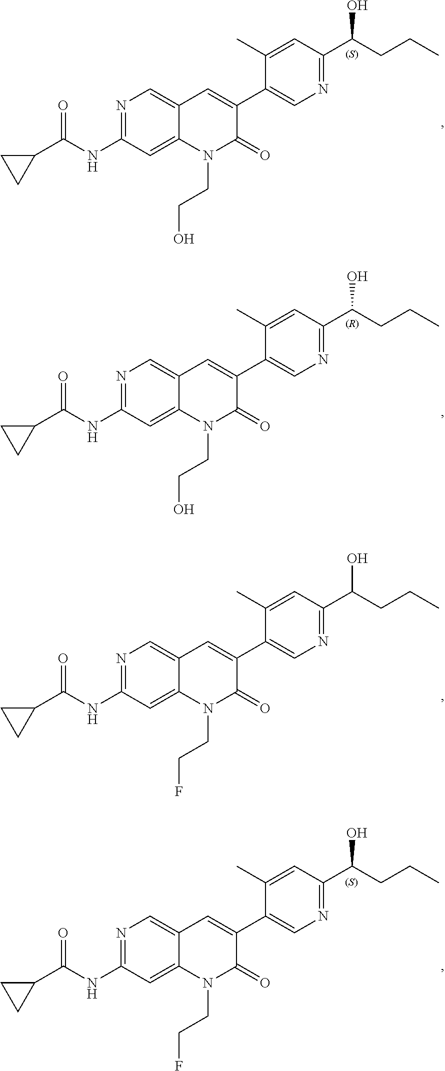

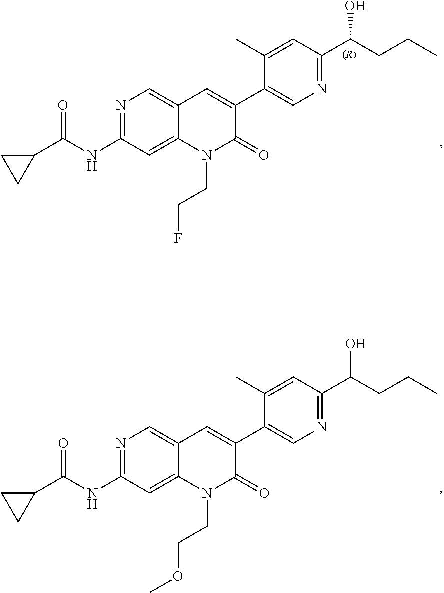

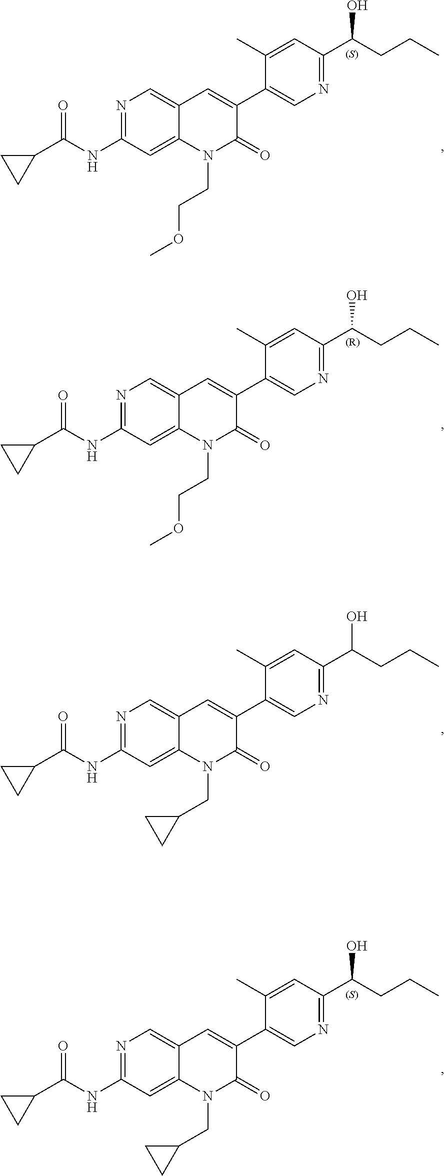

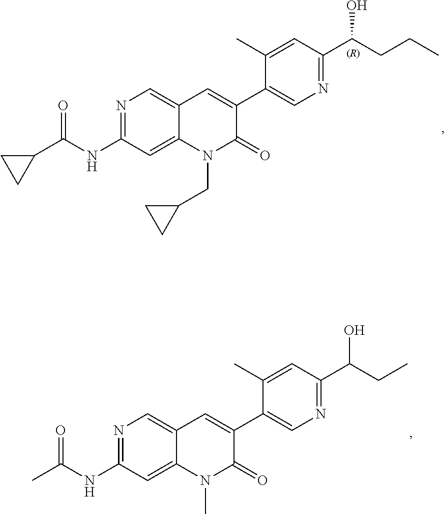

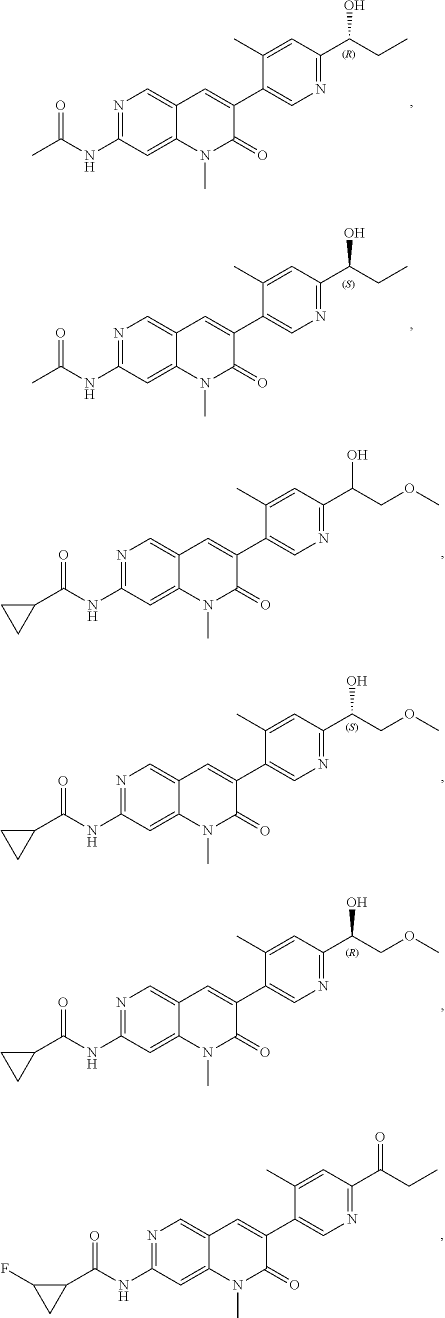

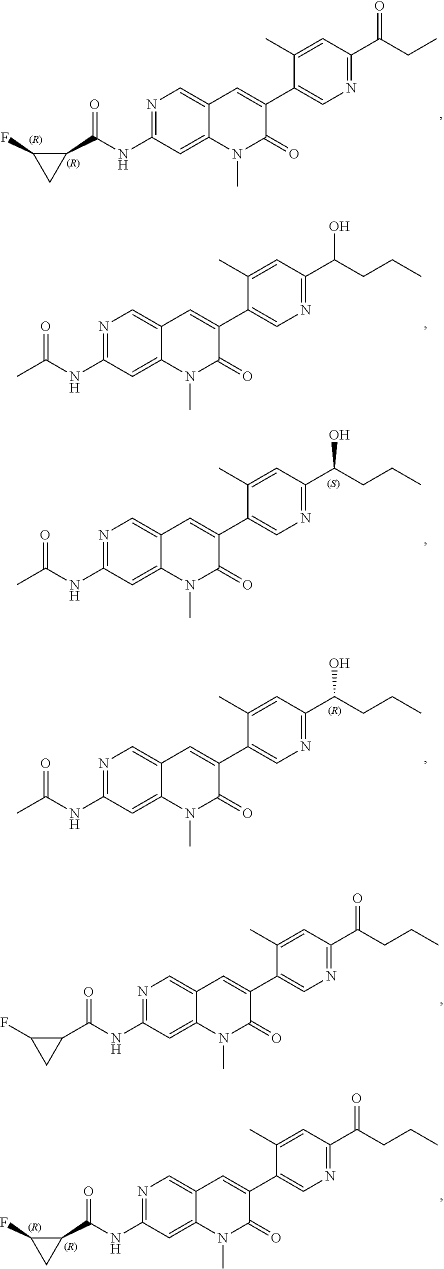

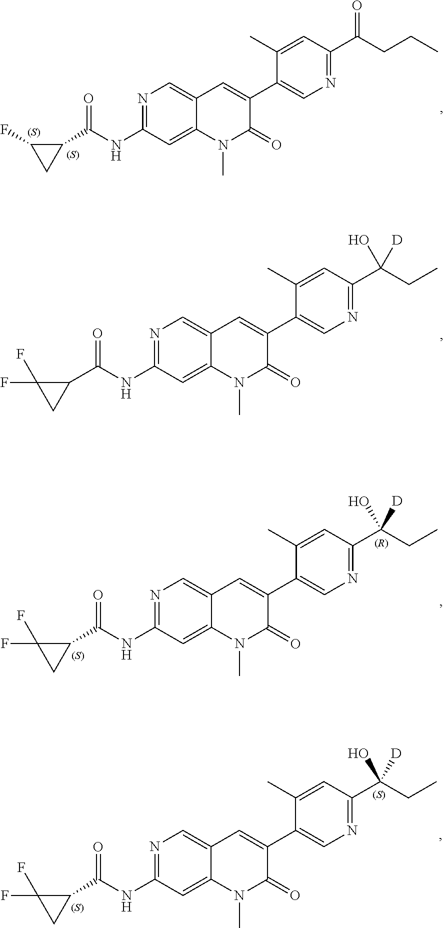

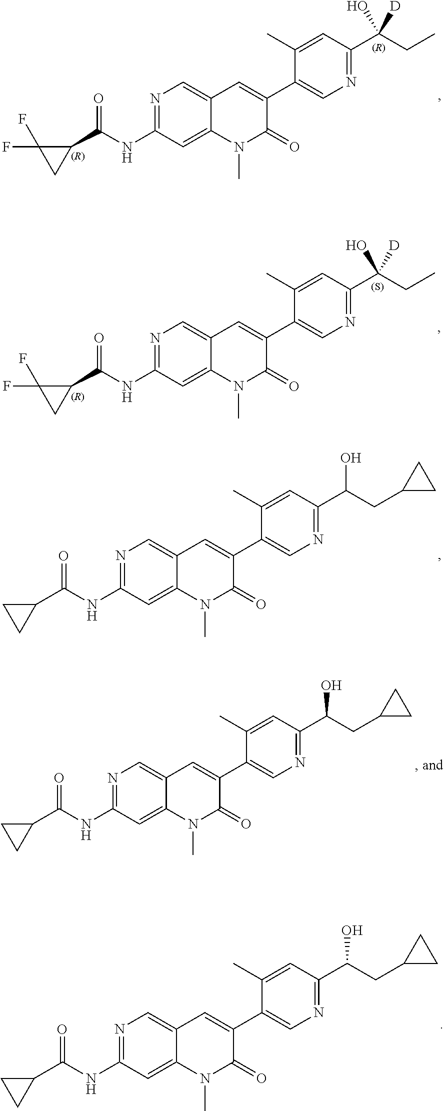

21. The compound of claims 1-2 and 5, or a pharmaceutically acceptable salt, solvate, hydrate, or co-crystal thereof, or a mixture of any of the foregoing, which is of formula (I-a), (I-b), (I-c), (I-d), (I-e), (I-f), or (I-g):

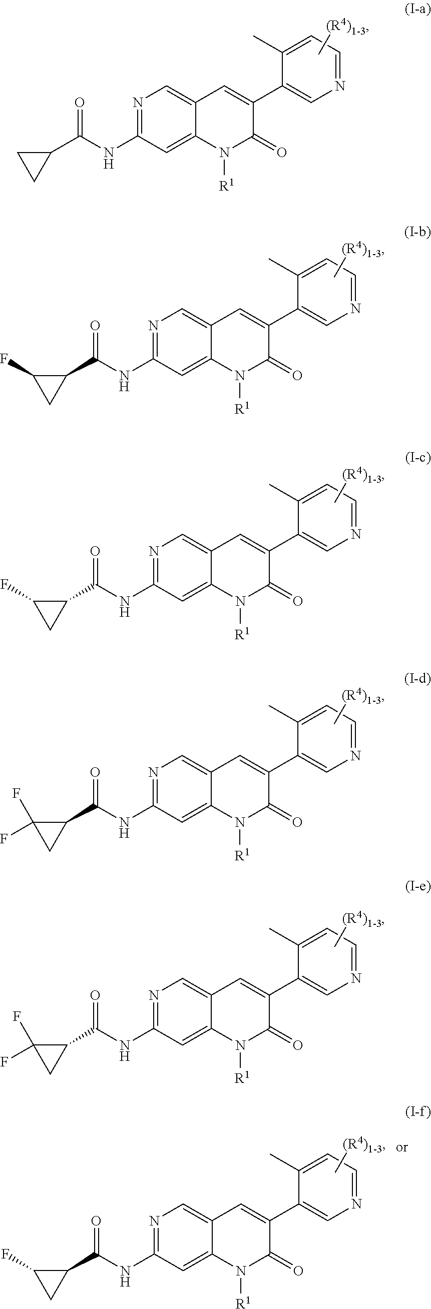

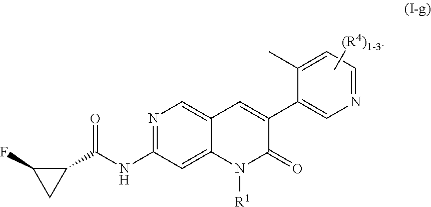

22. The compound of claim 21, or a pharmaceutically acceptable salt, solvate, hydrate, or co-crystal thereof, or a mixture of any of the foregoing, wherein: R¹ is H or —CH₃; and each R⁴ is independently H, F, —CH(OH)CH₃, —CH(OH)CH₂CH₃, —CH(OH)CH₂CH₂CH₃, —CD(OH)CH₂CH₂CH₃, —C(CH₃)₂OH, or —CH(OH)CF₃.

23. The compound of claim 21 or 22, or a pharmaceutically acceptable salt, solvate, hydrate, or co-crystal thereof, or a mixture of any of the foregoing, wherein:
R¹ is —CH₃; and
each R⁴ is independently H, F, —CH(OH)CH₃, —C(CH₃)₂OH, or —CH(OH)CF₃.

24. A pharmaceutical composition comprising the compound of any one of claims 1-23, or a pharmaceutically acceptable salt, solvate, hydrate, or co-crystal thereof, or a mixture of any of the foregoing, and one or more pharmaceutically acceptable excipients.

25. A method of inhibiting ARAF, BRAF and CRAF enzymatic activity in a cell, comprising exposing the cell with an effective amount of a compound of any one of claims 1-23, or a pharmaceutically acceptable salt, solvate, hydrate, or co-crystal thereof, or a mixture of any of the foregoing, or a pharmaceutical composition according to claim 24.

26. A method of treating a cancer or neoplastic disease in a human in need thereof, comprising administering to the human a compound of any one of claims 1-23, or a pharmaceutically acceptable salt, solvate, hydrate, or co-crystal thereof, or a mixture of any of the foregoing, or a pharmaceutical composition according to claim 24.

27. The method of claim 26, wherein the cancer or neoplastic disease is associated with one or more genetic alterations that engender elevated RAS/RAF/MEK/ERK pathway activation.

28. The method of claim 26 or 27, wherein the cancer or neoplastic disease is associated with one or more genetic alterations in KRAS, NRAS, HRAS, ARAF, BRAF or CRAF.

29. The method of any one of claims 26-28, wherein the cancer or neoplastic disease is associated with
one or more mutations in KRAS selected from the group consisting of G12D, G12V, G12C, G12S, G12R, G12A, G13D, G13C, G13R, Q61H, Q61K, Q61L, Q61P, Q61R and Q61E; or
one or more mutations in NRAS selected from the group consisting of G12D, G12S, G12C, G12V, G12A, G13D, G13R, G13V, G13C, G13A, G13S, G61R, Q61K Q61H, and G61L; or
one or more mutations in HRAS selected from the group consisting of G12V, G12S, G12D, G12C, G12R, G12A, G13R, G13V, G13D, G13S, G13C, Q61R, Q61L, Q61K, and Q61H; or
one or more mutations in ARAF selected from the group consisting of S214C and S214F; or
one or more mutations in BRAF selected from the group consisting of Class I, Class IIa, Class IIb, Class IIc, and Class III mutations; or
one or more mutations in CRAF selected from the group consisting of P261A, P261L, E478K, R391W, R391S and T491I, or a CRAF fusion.

30. The method of any one of claims 26-29, wherein the cancer or neoplastic disease is associated with one or more genetic lesions resulting in the activation of one or more receptor tyrosine kinases (RTKs).

31. The method of claim 30, wherein the one or more genetic lesions is a point mutation, a fusion or any combination thereof.

32. The method of claim 30 or 31, wherein the one or more receptor tyrosine kinase is selected from the group consisting of ALK, EGFR, ERBB2, LTK, MET, NTRK, RET, and ROS1.

33. The method of any one of claims 26-32, wherein the cancer is a refractory cancer.

34. The method of any one of claims 26-33, the refractory cancer is associated with one or more genetic alterations in BRAF selected from the group consisting of gene amplification, point mutation, BRAF fusion, and gene splicing events.

35. The method of any one of claims 26-34, the cancer is a refractory BRAF Class I mutant cancer.

36. The method of claim 35, wherein the refractory BRAF Class I mutant cancer is associated with a point mutation selected from the group consisting of V600D, V600E, V600K, and V600R.

37. The method of any one of claims 26-34, wherein the refractory cancer is associated with one or more Class II or Class III mutations in BRAF.

38. The method of claim 37, wherein the refractory cancer is associated with one or more mutations in BRAF selected from the group consisting of G464V, G469A, G469V, G469R, E586K, K601E, K601N, G466R, G466A, G466E, G466V, N581I, N581S, D594E, D594G, D594N, G596C, G596R, L597R, L597S, and L597Q.

39. The method of claim 37, wherein the refractory cancer is associated with one or more alternative splicing events that result in the loss of BRAF gene exons 4-10, 4-8, 2-8 or 2-10.

40. The method of any one of claims 26-39, wherein the cancer is a solid tumor or a hematological malignancy.

41. The method of claim 40, wherein the cancer is melanoma, lung cancer, pancreatic carcinoma, glioma, colorectal carcincoma, chronic myeloid leukemia (CML), acute myeloid leukemia (AML), or acute lymphoblastic leukemia (ALL).

42. The method of claim 41, wherein the lung cancer is non-small cell lung cancer (NSCLC).

43. The method of any one of claims 26-42, further comprising administering one or more pharmaceutical agents including anti-microtubular therapies, topoisomerase inhibitors, alkylating agents, nucleotide synthesis inhibitors, DNA synthesis inhibitors, protein synthesis inhibitors, developmental signaling pathway inhibitors, pro-apoptotic agents, RTK inhibitors, RAF inhibitors representing alternative binding modes, MEK1/2 inhibitors, ERK1/2 inhibitors, RSK1/2/3/4 inhibitors, AKT inhibitors, TORC1/2 inhibitors, DNA damage response pathway inhibitors, PI3K inhibitors and/or radiation.

44. A method of inhibiting Bcr-Abl enzymatic activity in a cell, comprising contacting the cell with an effective amount of the compound of any one of claims 1-23, or a pharmaceutically acceptable salt, solvate, hydrate, or co-crystal thereof, or a mixture of any of the foregoing, or the pharmaceutical composition of claim 24.

45. A method of treating chronic myeloid leukemia (CML), acute myeloid leukemia (AML), or acute lymphoblastic leukemia (ALL) in a human in need thereof, comprising administering to the human the compound of any one of claims 1-23, or a pharmaceutically acceptable salt, solvate, hydrate, or co-crystal thereof, or a mixture of any of the foregoing, or the pharmaceutical composition of claim 24.

46. The method of claim 45, wherein the chronic myeloid leukemia is refractory chronic myeloid leukemia.

47. The method of claim 46, wherein the refractory chronic myeloid leukemia is associated with a mutation selected from the group consisting of M244V, L248V, G250E, G250A, Q252H, Q252R, Y253F, Y253H, E255K, E255V, D276G, F311L, T315N, T315A, F317V, F317L, M343T, M351T, E355G, F359A, F359V, V379I, F382L, L387M, H396P, H396R, S417Y, E459K, F486S, and T315I.
